(19) United States
(12) Patent Application Publication
(10) Pub. No.: US 2024/0254099 A1
(43) Pub. Date: Aug. 1, 2024

(54) HETEROCYCLIC COMPOUND, AND ORGANIC LIGHT-EMITTING ELEMENT COMPRISING SAME

(71) Applicant: LT MATERIALS CO., LTD., Gyeonggi-do (KR)

(72) Inventors: Ji Un KIM, Yongin-si (KR); GWANG IL DONG, Yongin-si (KR); Yong Hui LEE, Yongin-si (KR); Won Jang JEONG, Yongin-si (KR); Dong Jun KIM, Yongin-si (KR)

(73) Assignee: LT MATERIALS CO., LTD., Yongin-si, Gyeonggi-do (KR)

(21) Appl. No.: 18/289,138

(22) PCT Filed: Apr. 8, 2022
(86) PCT No.: PCT/KR2022/005150

§ 371 Date: Nov. 1, 2023

(30) Foreign Application Priority Data
May 17, 2021 (KR) 10-2021-0063237

(51) Int. Cl. C07D 333/76; C07B 59/00; C07D 409/10; C07D 409/12; H10K 50/15; H10K 50/16; H10K 85/60

(52) U.S. Cl. CPC C07D 333/76; C07B 59/004; C07D 409/10; C07D 409/12; H10K 50/15; H10K 50/16; H10K 85/615; H10K 85/633; H10K 85/6574; H10K 85/6576; C07B 2200/05

(57) ABSTRACT

The present disclosure relates to a heterocyclic compound represented by Chemical Formula 1 and an organic light emitting device comprising the same.

### TECHNICAL FIELD

This application claims priority to and the benefits of Korean Patent Application No. 10-2021-0063237, filed with the Korean Intellectual Property Office on May 17, 2021, the entire contents of which are incorporated herein by reference.

The present disclosure relates to a heterocyclic compound and an organic light emitting device comprising the same.

### BACKGROUND ART

An organic light emitting device is one type of self-emissive display devices, and has advantages of having a wide viewing angle and a high response speed as well as having an excellent contrast.

An organic light emitting device has a structure of disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### DISCLOSURE

### Technical Problem

An object of the present disclosure is to provide a heterocyclic compound and an organic light emitting device comprising the same.

### Technical Solution

In order to achieve the above object, the present disclosure provides a heterocyclic compound represented by the following Chemical Formula 1.

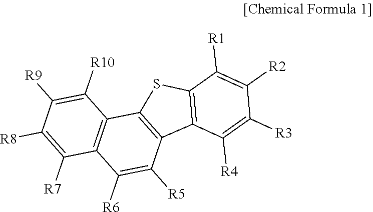

In Chemical Formula 1,
R1 to R10 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; a group represented by the following Chemical Formula 2; and a group represented by the following Chemical Formula 3, or two or more groups adjacent to each other bond to each other to form a substituted or unsubstituted C6 to C60 aromatic hydrocarbon ring; or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
at least one of R1 to R4 is a group represented by the following Chemical Formula 2; or a group represented by the following Chemical Formula 3, and
at least one of R5 to R10 is a group represented by the following Chemical Formula 2; or a group represented by the following Chemical Formula 3,

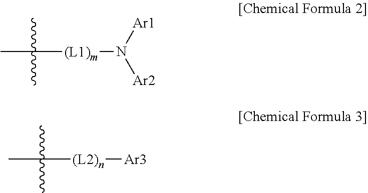

in Chemical Formula 2 and Chemical Formula 3,
Ar1 to Ar3 are the same as or different from each other, and each independently a substituted or unsubstituted C6 to C60 aryl group; or a substituted or unsubstituted C2 to C60 heteroaryl group,
L1 and L2 are the same as or different from each other, and each independently a direct bond; a substituted or unsubstituted C6 to C60 arylene group; or a substituted or unsubstituted C2 to C60 heteroarylene group, and
m and n are each an integer of 0 to 5, and when m is 2 or greater, L1s are the same as or different from each other, and when n is 2 or greater, L2s are the same as or different from each other.

In addition, the present disclosure provides an organic light emitting device comprising:
a first electrode;
a second electrode provided opposite to the first electrode; and
one or more organic material layers provided between the first electrode and the second electrode,
wherein at least one of the one or more layers of the organic material layers comprise the heterocyclic compound represented by Chemical Formula 1.

In addition, the present disclosure provides an organic light emitting device, wherein the organic material layer comprises a hole transport layer, and the hole transport layer comprises the heterocyclic compound represented by Chemical Formula 1.

In addition, one embodiment of the present disclosure provides an organic light emitting device, wherein the organic material layer comprises an electron blocking layer, and the electron blocking layer comprises the heterocyclic compound represented by Chemical Formula 1.

In addition, one embodiment of the present disclosure provides an organic light emitting device, wherein the organic material layer comprises a light emitting auxiliary layer, and the light emitting auxiliary layer comprises the heterocyclic compound represented by Chemical Formula 1.

### Advantageous Effects

A heterocyclic compound of the present disclosure can be used as a material of an organic material layer of an organic light emitting device. The heterocyclic compound can be used as a material of a hole injection layer, a hole transport layer, an electron blocking layer, a light emitting auxiliary layer, a light emitting layer, an electron transport layer, a hole blocking layer, an electron injection layer, a charge generation layer or the like in an organic light emitting device. Particularly, the heterocyclic compound represented by Chemical Formula 1 of the present disclosure can be used as a material of a hole transport layer, an electron blocking layer or a light emitting auxiliary layer of an organic light emitting device. Specifically, the heterocyclic compound represented by Chemical Formula 1 can be used as a hole transport layer material, or a material of an electron blocking layer or light emitting auxiliary layer either alone or as a combination with other compounds.

Specifically, the heterocyclic compound represented by Chemical Formula 1 is capable of enhancing a hole transport ability of a hole transport layer through adjusting a band gap and a T1 value, and, by improving molecular stability and stabilizing homo energy through delocalizing a homo energy level, is capable of lowering a driving voltage and enhancing light emission efficiency and lifetime properties in an organic light emitting device.

### DESCRIPTION OF DRAWINGS

FIG. 1 to FIG. 3 are diagrams each schematically illustrating a lamination structure of an organic light emitting device according to one embodiment of the present disclosure.

### MODE FOR DISCLOSURE

Hereinafter, the present disclosure will be described in more detail.

In the present specification, a term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; halogen; a cyano group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(—O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent linking two or more substituents selected from among the substituents illustrated above.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to and more specifically from 1 to 20. Specific examples thereof may include a methyl group, an ethyl group, an n-propyl group, an isopropyl group, an n-butyl group, an isobutyl group, a tert-butyl group, a sec-butyl group, a 1-methyl-butyl group, a 1-ethyl-butyl group, an n-pentyl group, an isopentyl group, a neopentyl group, a tert-pentyl group, an n-hexyl group, a 1-methylpentyl group, a 2-methylpentyl group, a 4-methyl-2-pentyl group, a 3,3-dimethylbutyl group, a 2-ethylbutyl group, an n-heptyl group, a 1-methylhexyl group, a cyclopentylmethyl group, a cyclohexylmethyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to and more specifically from 2 to 20.

In the present specification, the alkoxy group may be linear, branched or cyclic. The number of carbon atoms of the alkoxy group is not particularly limited, but is preferably from 1 to 20. Specific examples thereof may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatam, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, wherein R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be used. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. Wherein R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

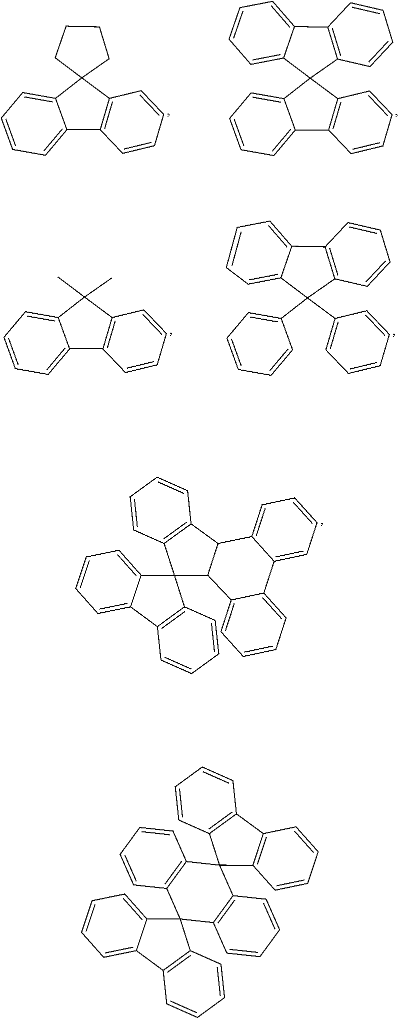

and the like may be included, however, the structure is not limited thereto.

In the present specification, the heteroaryl group includes S, O, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, a diazanaphthalenyl group, a triazaindenyl group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole group, a spirobi(dibenzosilole) group, a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions that may came as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be mixed in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

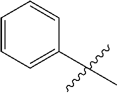 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulas.

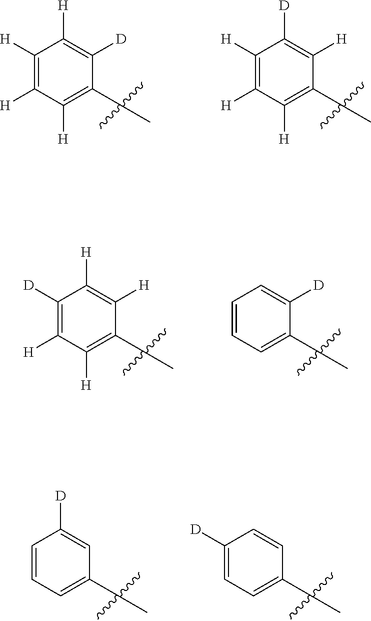 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include phenyl, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art satisfying the above-mentioned number of carbon atoms.

The present disclosure provides a heterocyclic compound represented by the following Chemical Formula 1.

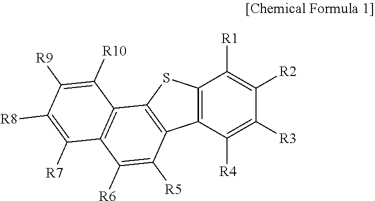

In Chemical Formula 1,
R1 to R10 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; a group represented by the following Chemical Formula 2; and a group represented by the following Chemical Formula 3, or two or more groups adjacent to each other bond to each other to form a substituted or unsubstituted C6 to C60 aromatic hydrocarbon ring; or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
at least one of R1 to R4 is a group represented by the following Chemical Formula 2; or a group represented by the following Chemical Formula 3, and
at least one of R5 to R10 is a group represented by the following Chemical Formula 2; or a group represented by the following Chemical Formula 3,

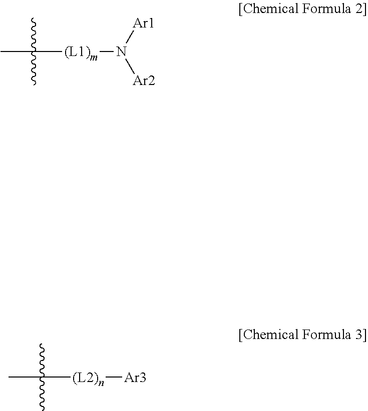

in Chemical Formula 2 and Chemical Formula 3,
Ar1 to Ar3 are the same as or different from each other, and each independently a substituted or unsubstituted C6 to C60 aryl group; or a substituted or unsubstituted C2 to C60 heteroaryl group,
L1 and L2 are the same as or different from each other, and each independently a direct bond; a substituted or unsubstituted C6 to C60 arylene group; or a substituted or unsubstituted C2 to C60 heteroarylene group, and
m and n are each an integer of 0 to 5, and when m is 2 or greater, L1s are the same as or different from each other, and when n is 2 or greater, L2s are the same as or different from each other.

In one embodiment of the present disclosure, R1 to R10 are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; the group represented by Chemical Formula 2; or the group represented by Chemical Formula 3.

In another embodiment of the present disclosure, R1 to R10 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; the group represented by Chemical Formula 2; or the group represented by Chemical Formula 3.

In another embodiment of the present disclosure, R1 to R10 are the same as or different from each other, and may be each independently hydrogen; deuterium; a substituted or unsubstituted C6 to C20 aryl group; a substituted or unsubstituted C2 to C20 heteroaryl group; the group represented by Chemical Formula 2; or the group represented by Chemical Formula 3.

In another embodiment of the present disclosure, R1 to R10 are the same as or different from each other, and may be each independently hydrogen; deuterium; the group represented by Chemical Formula 2; or the group represented by Chemical Formula 3.

In one embodiment of the present disclosure, the compound represented by Chemical Formula 1 may not include deuterium as a substituent, or a content of deuterium may be, for example, greater than 0%, 1% or greater, 10% or greater, 20% or greater, 30% or greater, 40% or greater or 50% or greater, and may be 100% or less, 90% or less, 80% or less, 70% or less or 60% or less based on the total number of hydrogen atoms and deuterium atoms.

In another embodiment of the present disclosure, the compound represented by Chemical Formula 1 may not include deuterium as a substituent, or a content of deuterium may be from 1% to 100% with respect to the total number of hydrogen atoms and deuterium atoms.

In another embodiment of the present disclosure, the compound represented by Chemical Formula 1 may not include deuterium as a substituent, or a content of deuterium may be from 20% to 100% with respect to the total number of hydrogen atoms and deuterium atoms.

In another embodiment of the present disclosure, the compound represented by Chemical Formula 1 may not include deuterium as a substituent, or a content of deuterium may be from 30% to 80% with respect to the total number of hydrogen atoms and deuterium atoms.

In another embodiment of the present disclosure, the compound represented by Chemical Formula 1 may not include deuterium as a substituent, or a content of deuterium may be from 50% to 70% with respect to the total number of hydrogen atoms and deuterium atoms.

In one embodiment of the present disclosure, Ar1 and Ar2 are the same as or different from each other, and may be each independently a substituted or unsubstituted C6 to C30 aryl group; or a substituted or unsubstituted C2 to C30 heteroaryl group.

In another embodiment of the present disclosure, Ar1 and Ar2 are the same as or different from each other, and may be each independently a substituted or unsubstituted C6 to C20 aryl group; or a substituted or unsubstituted C2 to C20 heteroaryl group.

In another embodiment of the present disclosure, Ar1 and Ar2 are the same as or different from each other, and may be each independently a substituted or unsubstituted phenyl group; a substituted or unsubstituted naphthyl group; a substituted or unsubstituted biphenyl group; a substituted or unsubstituted terphenyl group; a substituted or unsubstituted fluorenyl group; a substituted or unsubstituted spirobifluorenyl group; or a substituted or unsubstituted dibenzofuranyl group.

In one embodiment of the present disclosure, Ar3 may be a substituted or unsubstituted C6 to C30 aryl group; or a substituted or unsubstituted C2 to C30 heteroaryl group.

In another embodiment of the present disclosure, Ar3 may be a substituted or unsubstituted C6 to C20 aryl group; or a substituted or unsubstituted C2 to C20 heteroaryl group.

In another embodiment of the present disclosure, Ar3 may be a substituted or unsubstituted phenyl group; a substituted or unsubstituted naphthyl group; a substituted or unsubstituted biphenyl group; a substituted or unsubstituted terphenyl group; a substituted or unsubstituted fluorenyl group; a substituted or unsubstituted spirobifluorenyl group; a substituted or unsubstituted dibenzofuranyl group; or a substituted or unsubstituted dibenzothiophenyl group.

In one embodiment of the present disclosure, L1 and L2 are the same as or different from each other, and may be each independently a direct bond; a substituted or unsubstituted C6 to C30 arylene group; or a substituted or unsubstituted C2 to C30 heteroarylene group.

In another embodiment of the present disclosure, L1 and L2 are the same as or different from each other, and may be each independently a direct bond; a substituted or unsubstituted C6 to C20 arylene group; or a substituted or unsubstituted C2 to C20 heteroarylene group.

In another embodiment of the present disclosure, L1 and L2 are the same as or different from each other, and may be each independently a direct bond; or a substituted or unsubstituted phenylene group.

Specific examples of L1 and L2 are shown below, however, L1 and L2 are not limited to these examples.

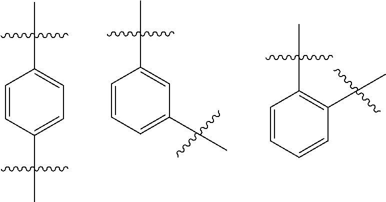

In one embodiment of the present disclosure, Chemical Formula 1 may be a heterocyclic compound represented by the following Chemical Formula 4 or Chemical Formula 5.

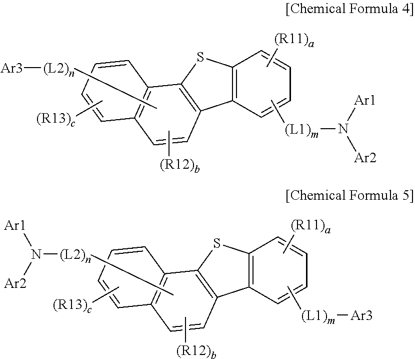

In Chemical Formulas 4 and 5,
R11 to R13 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(—O)R101R102; and —SiR101R102R103, or two or more groups adjacent to each other bond to each other to form a substituted or unsubstituted C6 to C60 aromatic hydrocarbon ring; or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
a is an integer of 0 to 3, and when a is 2 or greater, R11s are the same as or different from each other,
b is an integer of 0 to 2, and when b is 2 or greater, R12s are the same as or different from each other,
c is an integer of 0 to 4, and when c is 2 or greater, R13s are the same as or different from each other,
Ar1, Ar2, L1 and m have the same definitions as in Chemical Formula 2, and
Ar3, L2 and n have the same definitions as in Chemical Formula 3.

In one embodiment of the present disclosure, R11 to R13 are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; or —SiR101R102R103.

In another embodiment of the present disclosure, R11 to R13 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; or —SiR101R102R103.

In another embodiment of the present disclosure, R11 to R13 are the same as or different from each other, and may be each independently hydrogen; or deuterium.

In one embodiment of the present disclosure, Chemical Formula 1 may be a heterocyclic compound of the following Chemical Formula 6 or Chemical Formula 7.

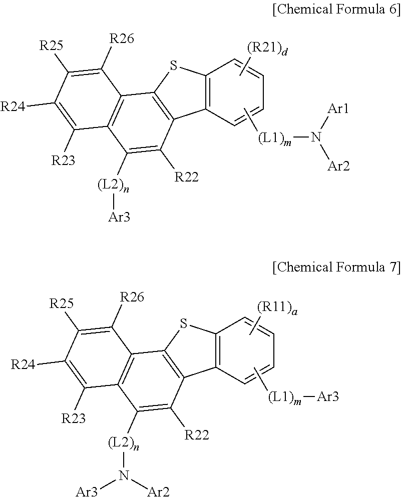

In Chemical Formulas 6 and 7,
R21 to R26 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, or two or more groups adjacent to each other bond to each other to form a substituted or unsubstituted C6 to C60 aromatic hydrocarbon ring; or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
d is an integer of 0 to 3, and when d is 2 or greater, R21s are the same as or different from each other,
Ar1, Ar2, L1 and m have the same definitions as in Chemical Formula 2, and
Ar3, L2 and n have the same definitions as in Chemical Formula 3.

In one embodiment of the present disclosure, R21 to R26 are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(—O)R101R102; or —SiR101R102R103.

In another embodiment of the present disclosure, R21 to R26 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(—O)R101R102; or —SiR101R102R103.

In another embodiment of the present disclosure, R21 to R26 are the same as or different from each other, and may be each independently hydrogen; or deuterium.

In one embodiment of the present disclosure, Chemical Formula 1 may be a heterocyclic compound represented by any one of the following compounds.

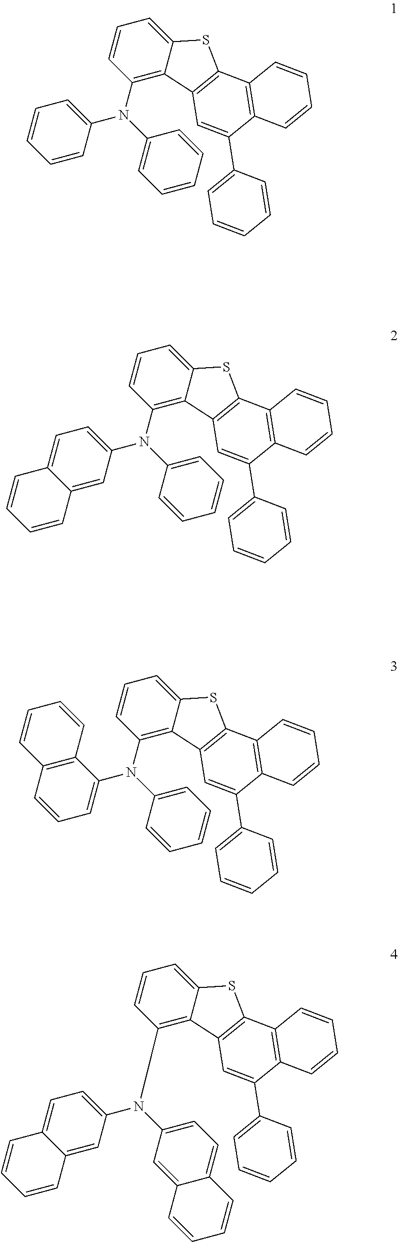 [SMILES: c1ccc(-c2cc3c(sc4cccc(N(c5ccccc5)c5ccccc5)c43)c3ccccc23)cc1 ; c1ccc(-c2cc3c(sc4cccc(N(c5ccccc5)c5ccc6ccccc6c5)c43)c3ccccc23)cc1 ; c1ccc(-c2cc3c(sc4cccc(N(c5ccccc5)c5cccc6ccccc56)c43)c3ccccc23)cc1 ; c1ccc(-c2cc3c(sc4cccc(N(c5ccc6ccccc6c5)c5ccc6ccccc6c5)c43)c3ccccc23)cc1]

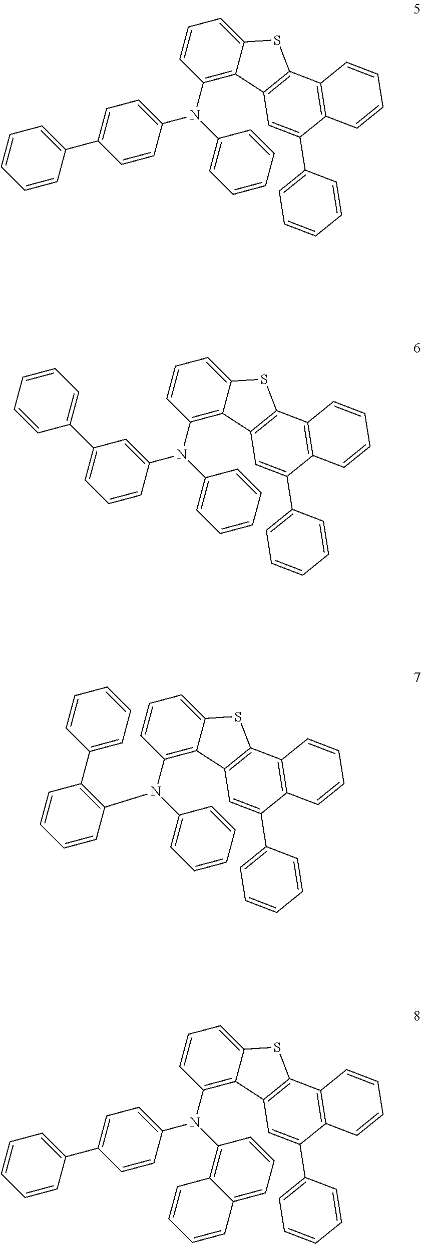 [SMILES: c1ccc(-c2ccc(N(c3ccccc3)c3cccc4sc5c6ccccc6c(-c6ccccc6)cc5c34)cc2)cc1 ; c1ccc(-c2cccc(N(c3ccccc3)c3cccc4sc5c6ccccc6c(-c6ccccc6)cc5c34)c2)cc1 ; c1ccc(-c2ccccc2N(c2ccccc2)c2cccc3sc4c5ccccc5c(-c5ccccc5)cc4c23)cc1 ; c1ccc(-c2ccc(N(c3cccc4ccccc34)c3cccc4sc5c6ccccc6c(-c6ccccc6)cc5c34)cc2)cc1]

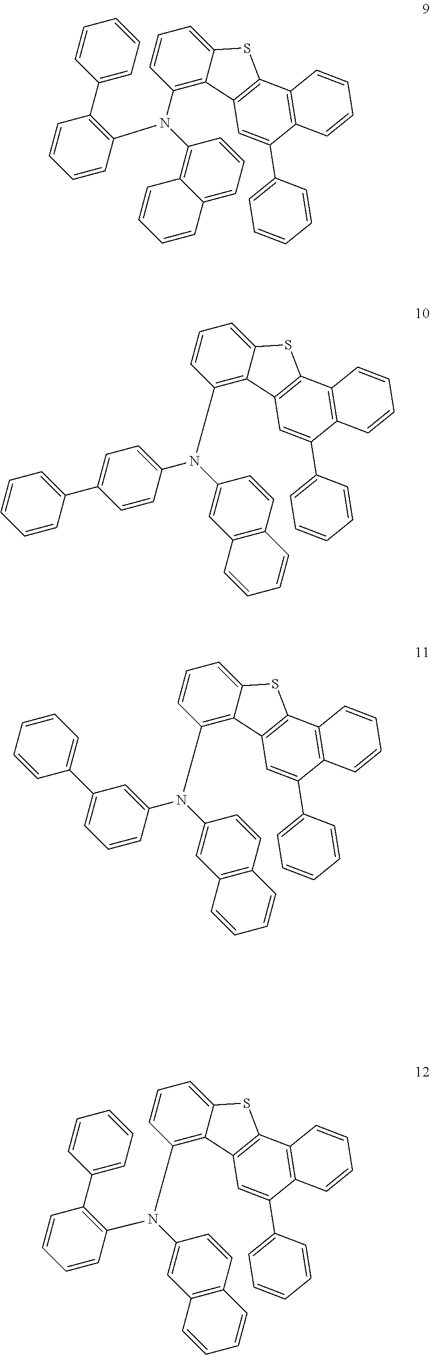 [SMILES: c1ccc(-c2ccccc2N(c2cccc3ccccc23)c2cccc3sc4c5ccccc5c(-c5ccccc5)cc4c23)cc1 ; c1ccc(-c2ccc(N(c3ccc4ccccc4c3)c3cccc4sc5c6ccccc6c(-c6ccccc6)cc5c34)cc2)cc1 ; c1ccc(-c2cccc(N(c3ccc4ccccc4c3)c3cccc4sc5c6ccccc6c(-c6ccccc6)cc5c34)c2)cc1 ; c1ccc(-c2ccccc2N(c2ccc3ccccc3c2)c2cccc3sc4c5ccccc5c(-c5ccccc5)cc4c23)cc1]

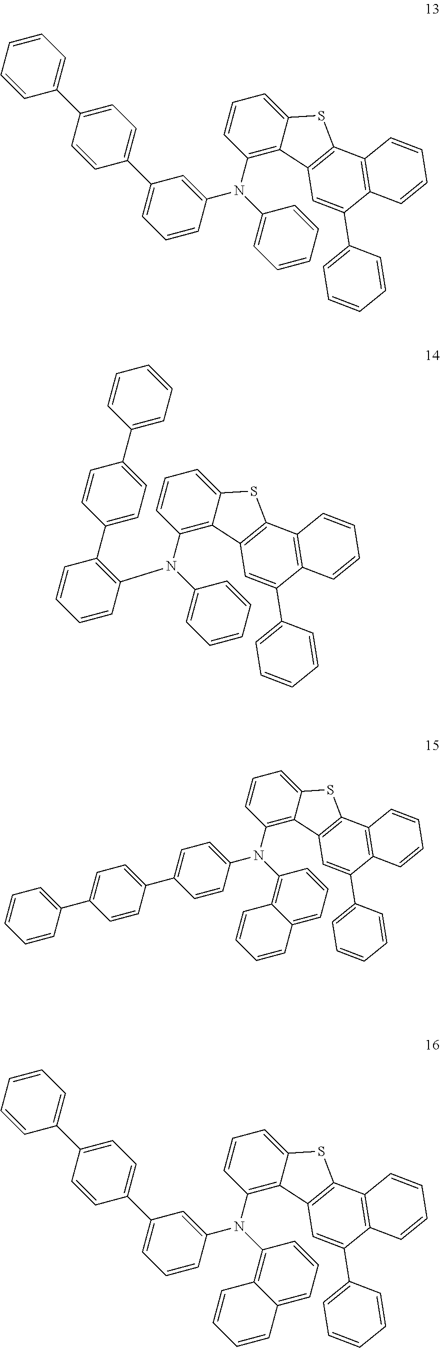 [SMILES: c1ccc(-c2ccc(-c3cccc(N(c4ccccc4)c4cccc5sc6c7ccccc7c(-c7ccccc7)cc6c45)c3)cc2)cc1 ; c1ccc(-c2ccc(-c3ccccc3N(c3ccccc3)c3cccc4sc5c6ccccc6c(-c6ccccc6)cc5c34)cc2)cc1 ; c1ccc(-c2ccc(-c3ccc(N(c4cccc5ccccc45)c4cccc5sc6c7ccccc7c(-c7ccccc7)cc6c45)cc3)cc2)cc1 ; c1ccc(-c2ccc(-c3cccc(N(c4cccc5ccccc45)c4cccc5sc6c7ccccc7c(-c7ccccc7)cc6c45)c3)cc2)cc1]

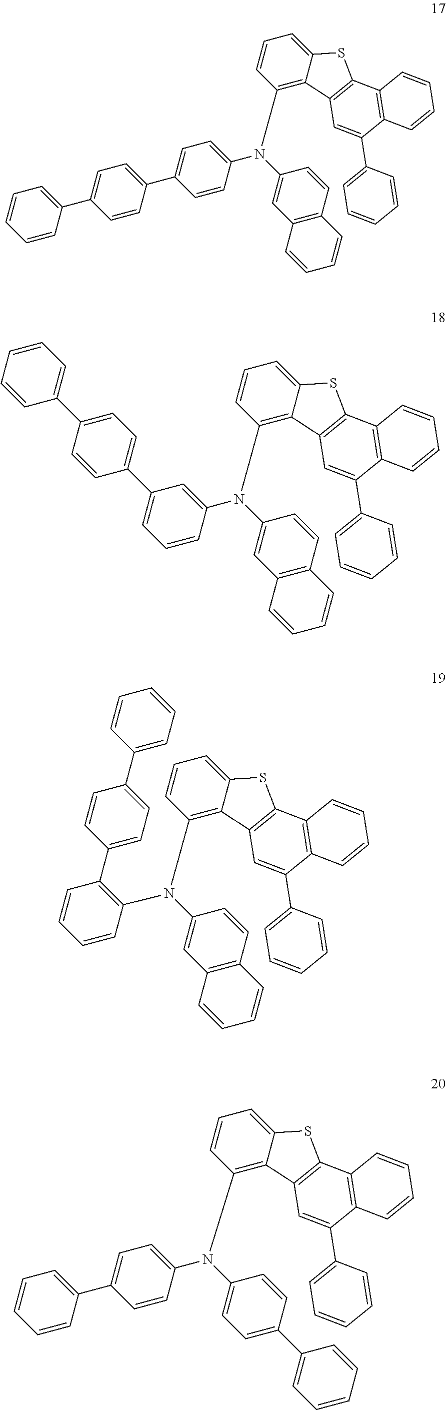 [SMILES: c1ccc(-c2ccc(-c3ccc(N(c4ccc5ccccc5c4)c4cccc5sc6c7ccccc7c(-c7ccccc7)cc6c45)cc3)cc2)cc1 ; c1ccc(-c2ccc(-c3cccc(N(c4ccc5ccccc5c4)c4cccc5sc6c7ccccc7c(-c7ccccc7)cc6c45)c3)cc2)cc1 ; c1ccc(-c2ccc(-c3ccccc3N(c3ccc4ccccc4c3)c3cccc4sc5c6ccccc6c(-c6ccccc6)cc5c34)cc2)cc1 ; c1ccc(-c2ccc(N(c3ccc(-c4ccccc4)cc3)c3cccc4sc5c6ccccc6c(-c6ccccc6)cc5c34)cc2)cc1]

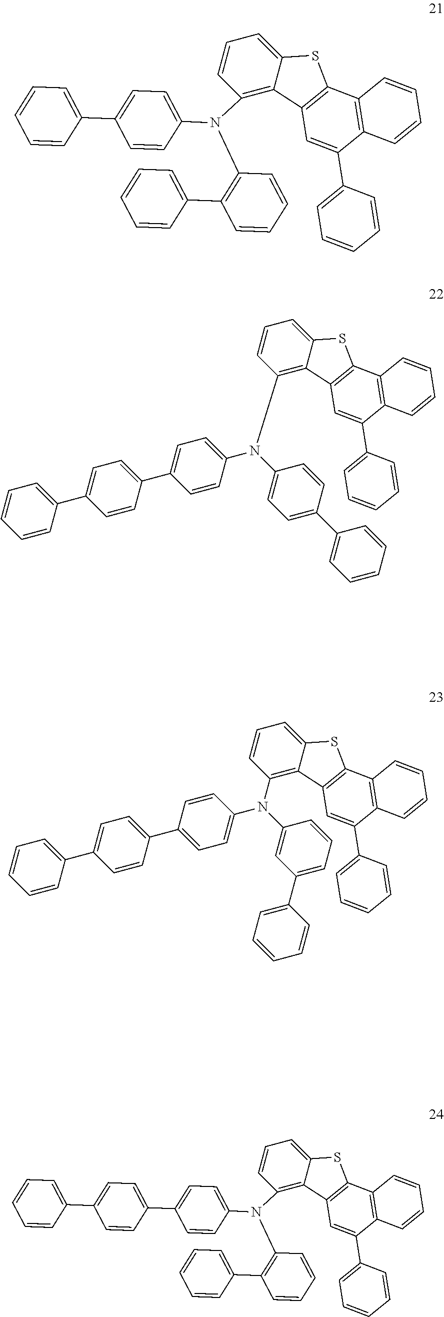 [SMILES: c1ccc(-c2ccc(N(c3ccccc3-c3ccccc3)c3cccc4sc5c6ccccc6c(-c6ccccc6)cc5c34)cc2)cc1 ; c1ccc(-c2ccc(-c3ccc(N(c4ccc(-c5ccccc5)cc4)c4cccc5sc6c7ccccc7c(-c7ccccc7)cc6c45)cc3)cc2)cc1 ; c1ccc(-c2ccc(-c3ccc(N(c4cccc(-c5ccccc5)c4)c4cccc5sc6c7ccccc7c(-c7ccccc7)cc6c45)cc3)cc2)cc1 ; c1ccc(-c2ccc(-c3ccc(N(c4ccccc4-c4ccccc4)c4cccc5sc6c7ccccc7c(-c7ccccc7)cc6c45)cc3)cc2)cc1]

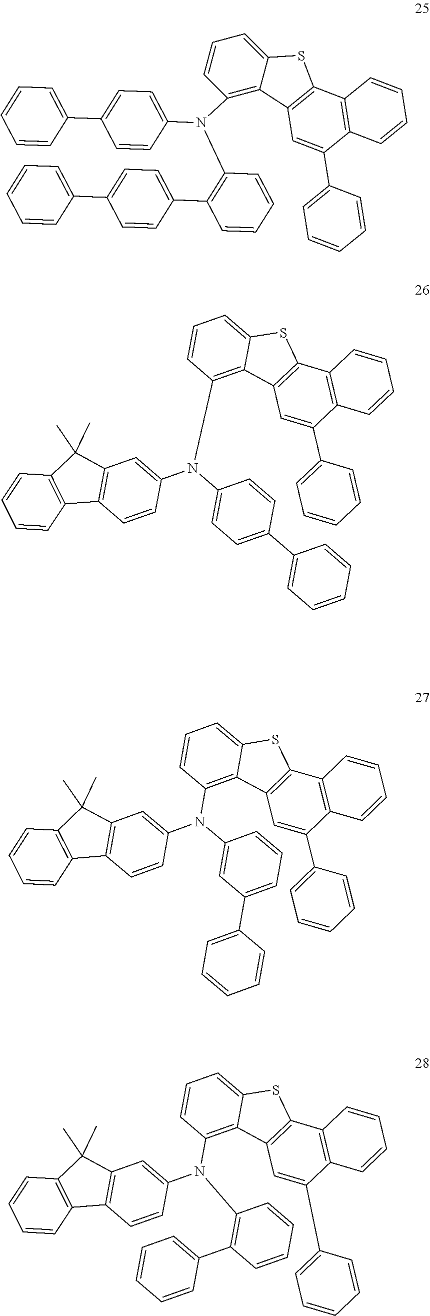 [SMILES: c1ccc(-c2ccc(-c3ccccc3N(c3ccc(-c4ccccc4)cc3)c3cccc4sc5c6ccccc6c(-c6ccccc6)cc5c34)cc2)cc1 ; CC1(C)c2ccccc2-c2ccc(N(c3ccc(-c4ccccc4)cc3)c3cccc4sc5c6ccccc6c(-c6ccccc6)cc5c34)cc21 ; CC1(C)c2ccccc2-c2ccc(N(c3cccc(-c4ccccc4)c3)c3cccc4sc5c6ccccc6c(-c6ccccc6)cc5c34)cc21 ; CC1(C)c2ccccc2-c2ccc(N(c3ccccc3-c3ccccc3)c3cccc4sc5c6ccccc6c(-c6ccccc6)cc5c34)cc21]

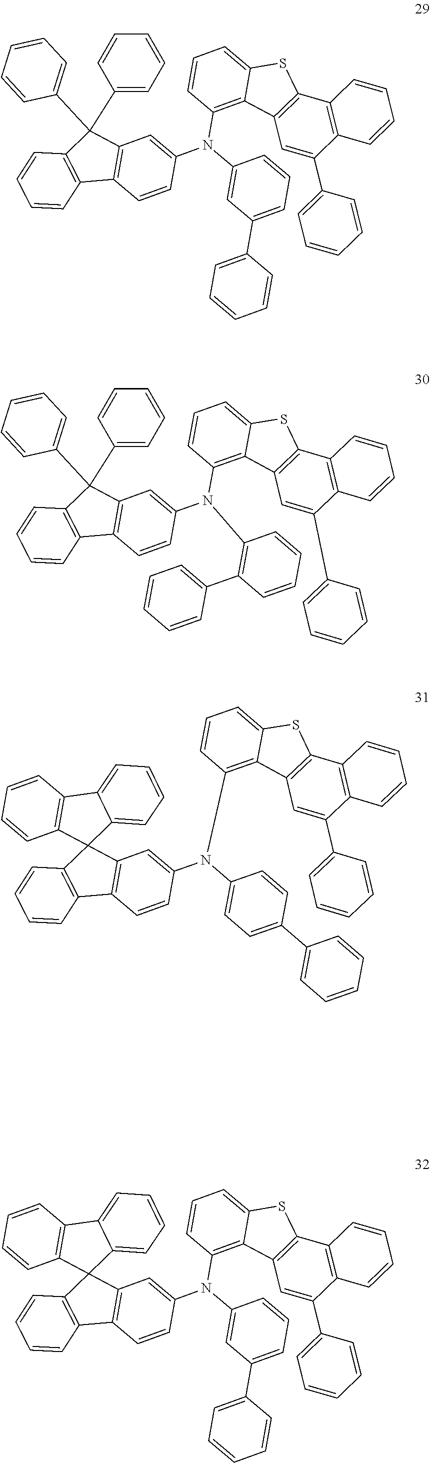 [SMILES: c1ccc(-c2cccc(N(c3ccc4c(c3)C(c3ccccc3)(c3ccccc3)c3ccccc3-4)c3cccc4sc5c6ccccc6c(-c6ccccc6)cc5c34)c2)cc1 ; c1ccc(-c2ccccc2N(c2ccc3c(c2)C(c2ccccc2)(c2ccccc2)c2ccccc2-3)c2cccc3sc4c5ccccc5c(-c5ccccc5)cc4c23)cc1 ; c1ccc(-c2ccc(N(c3ccc4c(c3)C3(c5ccccc5-c5ccccc53)c3ccccc3-4)c3cccc4sc5c6ccccc6c(-c6ccccc6)cc5c34)cc2)cc1 ; c1ccc(-c2cccc(N(c3ccc4c(c3)C3(c5ccccc5-c5ccccc53)c3ccccc3-4)c3cccc4sc5c6ccccc6c(-c6ccccc6)cc5c34)c2)cc1]

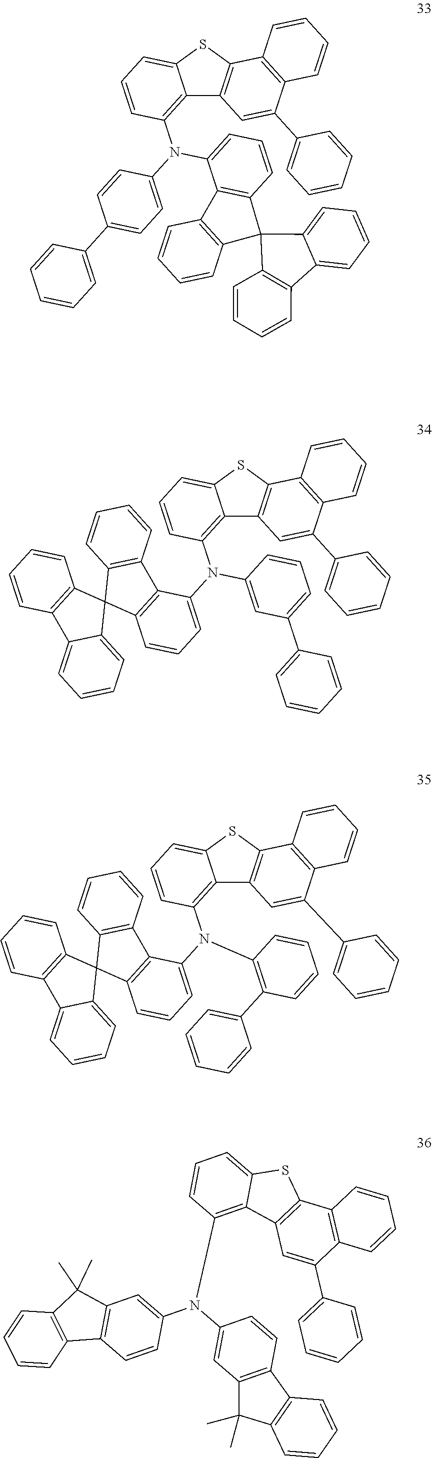 [SMILES: c1ccc(-c2ccc(N(c3cccc4c3-c3ccccc3C43c4ccccc4-c4ccccc43)c3cccc4sc5c6ccccc6c(-c6ccccc6)cc5c34)cc2)cc1 ; c1ccc(-c2cccc(N(c3cccc4c3-c3ccccc3C43c4ccccc4-c4ccccc43)c3cccc4sc5c6ccccc6c(-c6ccccc6)cc5c34)c2)cc1 ; c1ccc(-c2ccccc2N(c2cccc3c2-c2ccccc2C32c3ccccc3-c3ccccc32)c2cccc3sc4c5ccccc5c(-c5ccccc5)cc4c23)cc1 ; CC1(C)c2ccccc2-c2ccc(N(c3ccc4c(c3)C(C)(C)c3ccccc3-4)c3cccc4sc5c6ccccc6c(-c6ccccc6)cc5c34)cc21]

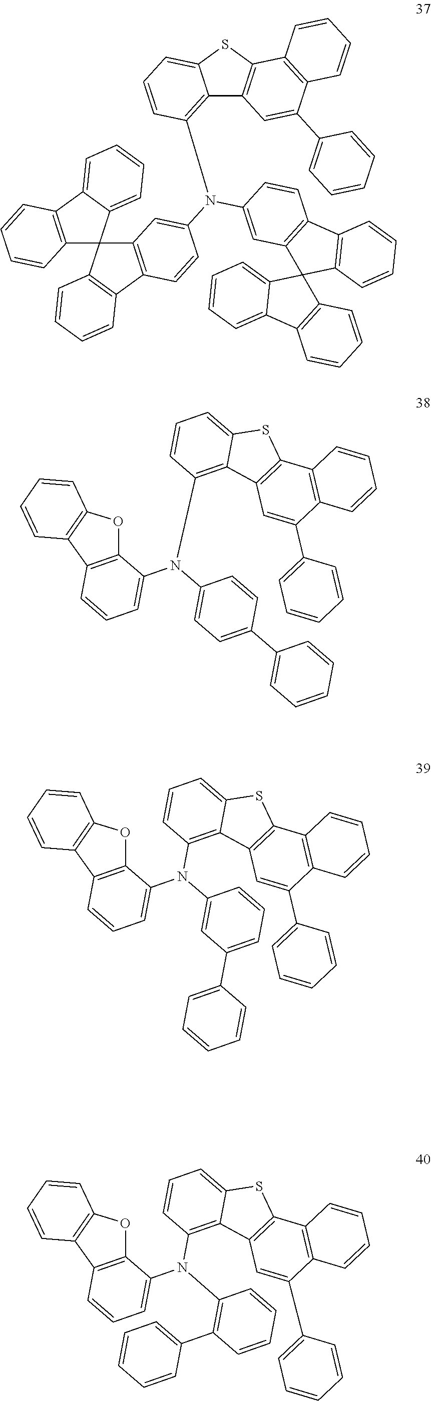 [SMILES: c1ccc(-c2cc3c(sc4cccc(N(c5ccc6c(c5)C5(c7ccccc7-c7ccccc75)c5ccccc5-6)c5ccc6c(c5)C5(c7ccccc7-c7ccccc75)c5ccccc5-6)c43)c3ccccc23)cc1 ; c1ccc(-c2ccc(N(c3cccc4c3oc3ccccc34)c3cccc4sc5c6ccccc6c(-c6ccccc6)cc5c34)cc2)cc1 ; c1ccc(-c2cccc(N(c3cccc4c3oc3ccccc34)c3cccc4sc5c6ccccc6c(-c6ccccc6)cc5c34)c2)cc1 ; c1ccc(-c2ccccc2N(c2cccc3c2oc2ccccc23)c2cccc3sc4c5ccccc5c(-c5ccccc5)cc4c23)cc1]

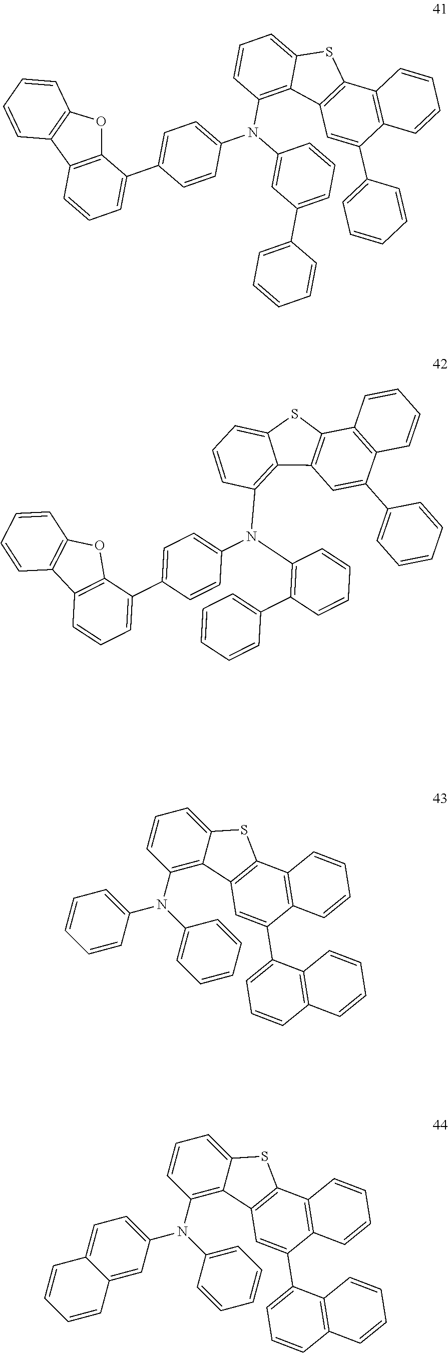 [SMILES: c1ccc(-c2cccc(N(c3ccc(-c4cccc5c4oc4ccccc45)cc3)c3cccc4sc5c6ccccc6c(-c6ccccc6)cc5c34)c2)cc1 ; c1ccc(-c2ccccc2N(c2ccc(-c3cccc4c3oc3ccccc34)cc2)c2cccc3sc4c5ccccc5c(-c5ccccc5)cc4c23)cc1 ; c1ccc(N(c2ccccc2)c2cccc3sc4c5ccccc5c(-c5cccc6ccccc56)cc4c23)cc1 ; c1ccc(N(c2ccc3ccccc3c2)c2cccc3sc4c5ccccc5c(-c5cccc6ccccc56)cc4c23)cc1]

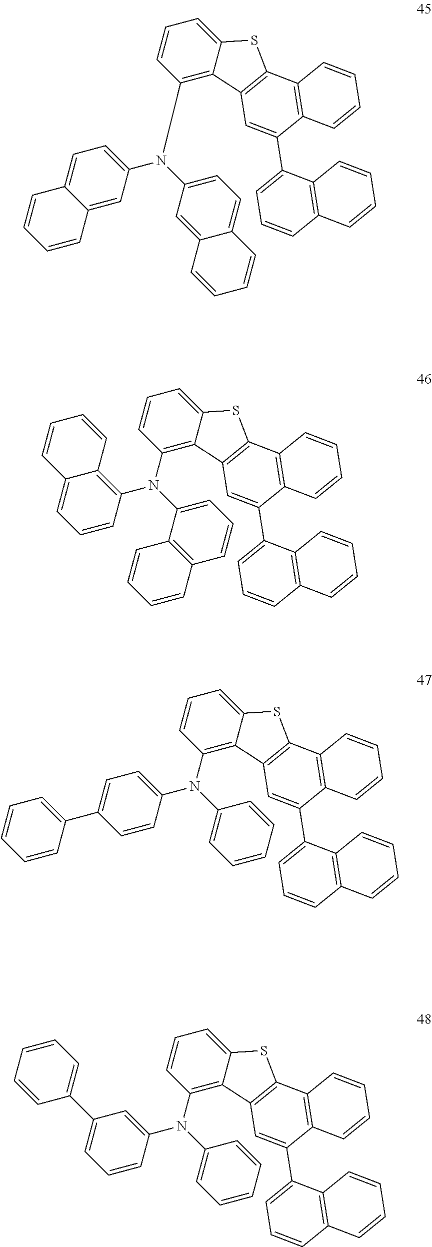 [SMILES: c1ccc2cc(N(c3ccc4ccccc4c3)c3cccc4sc5c6ccccc6c(-c6cccc7ccccc67)cc5c34)ccc2c1 ; c1ccc2c(-c3cc4c(sc5cccc(N(c6cccc7ccccc67)c6cccc7ccccc67)c54)c4ccccc34)cccc2c1 ; c1ccc(-c2ccc(N(c3ccccc3)c3cccc4sc5c6ccccc6c(-c6cccc7ccccc67)cc5c34)cc2)cc1 ; c1ccc(-c2cccc(N(c3ccccc3)c3cccc4sc5c6ccccc6c(-c6cccc7ccccc67)cc5c34)c2)cc1]

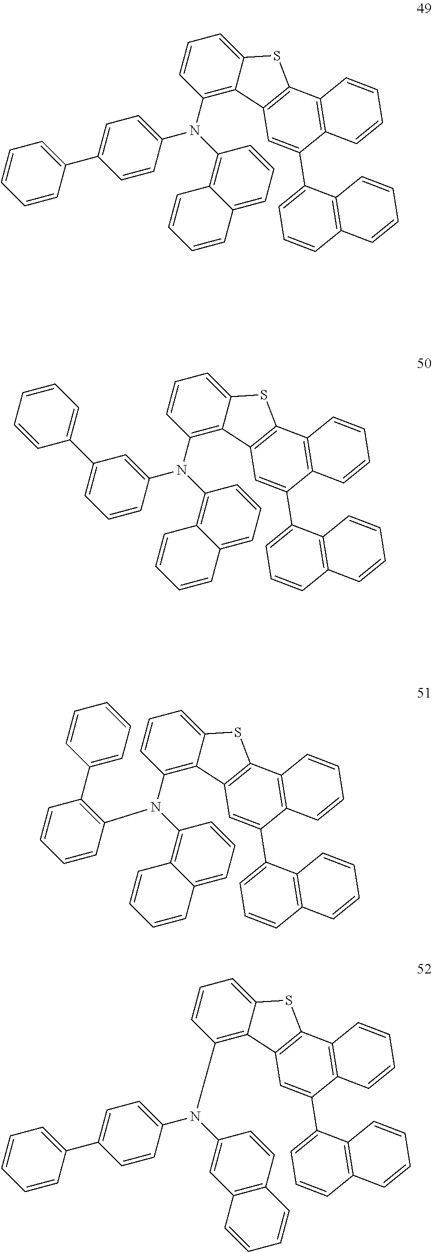 [SMILES: c1ccc(-c2ccc(N(c3cccc4ccccc34)c3cccc4sc5c6ccccc6c(-c6cccc7ccccc67)cc5c34)cc2)cc1 ; c1ccc(-c2cccc(N(c3cccc4ccccc34)c3cccc4sc5c6ccccc6c(-c6cccc7ccccc67)cc5c34)c2)cc1 ; c1ccc(-c2ccccc2N(c2cccc3ccccc23)c2cccc3sc4c5ccccc5c(-c5cccc6ccccc56)cc4c23)cc1 ; c1ccc(-c2ccc(N(c3ccc4ccccc4c3)c3cccc4sc5c6ccccc6c(-c6cccc7ccccc67)cc5c34)cc2)cc1]

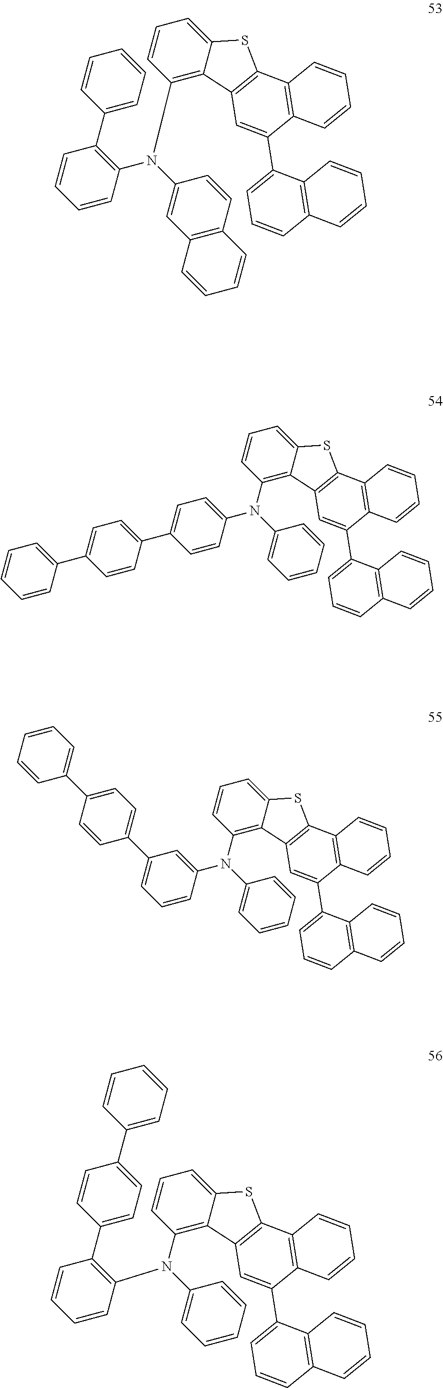 [SMILES: c1ccc(-c2ccccc2N(c2ccc3ccccc3c2)c2cccc3sc4c5ccccc5c(-c5cccc6ccccc56)cc4c23)cc1 ; c1ccc(-c2ccc(-c3ccc(N(c4ccccc4)c4cccc5sc6c7ccccc7c(-c7cccc8ccccc78)cc6c45)cc3)cc2)cc1 ; c1ccc(-c2ccc(-c3cccc(N(c4ccccc4)c4cccc5sc6c7ccccc7c(-c7cccc8ccccc78)cc6c45)c3)cc2)cc1 ; c1ccc(-c2ccc(-c3ccccc3N(c3ccccc3)c3cccc4sc5c6ccccc6c(-c6cccc7ccccc67)cc5c34)cc2)cc1]

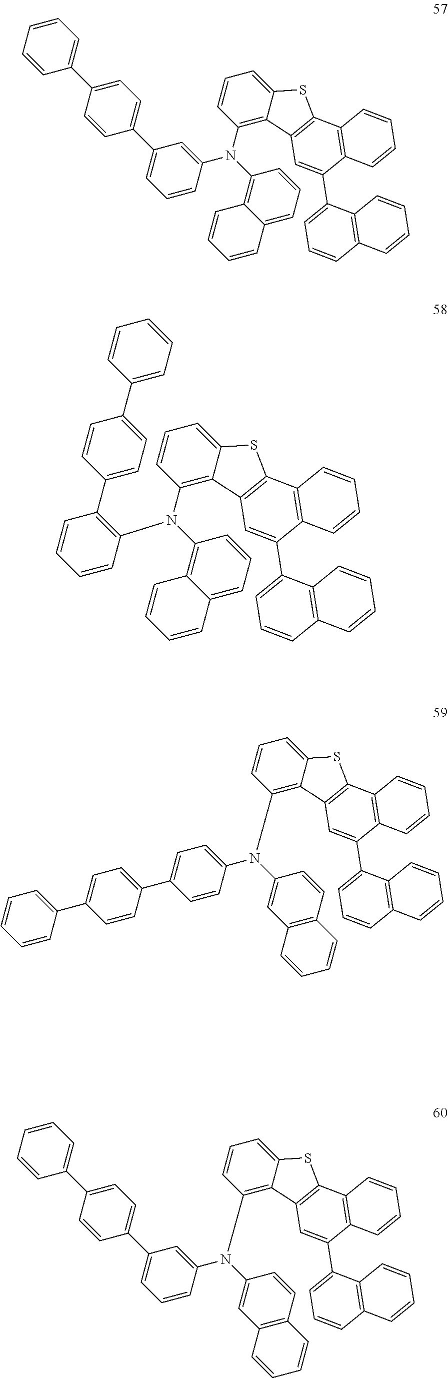 [SMILES: c1ccc(-c2ccc(-c3cccc(N(c4cccc5ccccc45)c4cccc5sc6c7ccccc7c(-c7cccc8ccccc78)cc6c45)c3)cc2)cc1 ; c1ccc(-c2ccc(-c3ccccc3N(c3cccc4ccccc34)c3cccc4sc5c6ccccc6c(-c6cccc7ccccc67)cc5c34)cc2)cc1 ; c1ccc(-c2ccc(-c3ccc(N(c4ccc5ccccc5c4)c4cccc5sc6c7ccccc7c(-c7cccc8ccccc78)cc6c45)cc3)cc2)cc1 ; c1ccc(-c2ccc(-c3cccc(N(c4ccc5ccccc5c4)c4cccc5sc6c7ccccc7c(-c7cccc8ccccc78)cc6c45)c3)cc2)cc1]

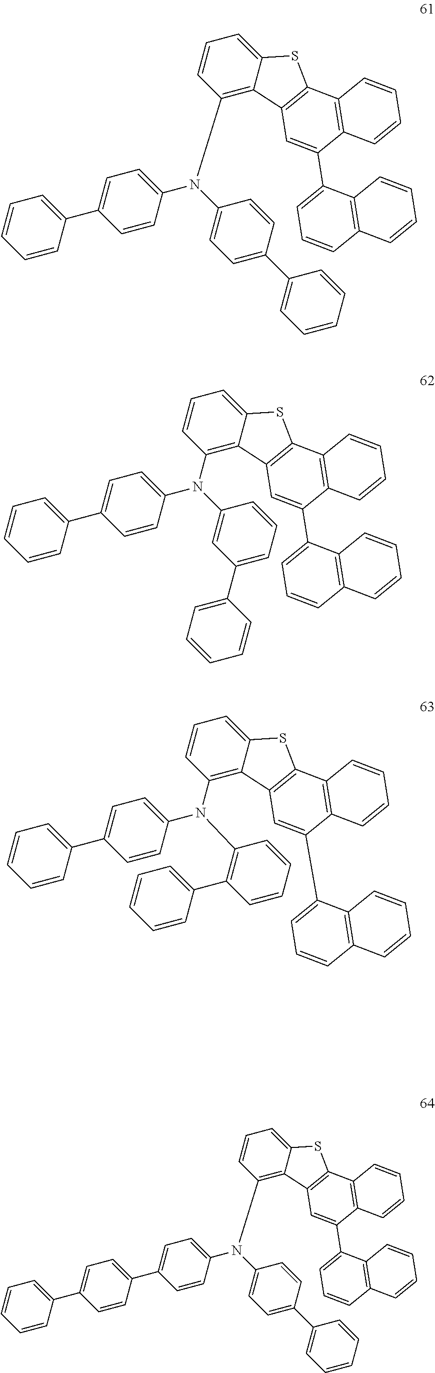 [SMILES: c1ccc(-c2ccc(N(c3ccc(-c4ccccc4)cc3)c3cccc4sc5c6ccccc6c(-c6cccc7ccccc67)cc5c34)cc2)cc1 ; c1ccc(-c2ccc(N(c3cccc(-c4ccccc4)c3)c3cccc4sc5c6ccccc6c(-c6cccc7ccccc67)cc5c34)cc2)cc1 ; c1ccc(-c2ccc(N(c3ccccc3-c3ccccc3)c3cccc4sc5c6ccccc6c(-c6cccc7ccccc67)cc5c34)cc2)cc1 ; c1ccc(-c2ccc(-c3ccc(N(c4ccc(-c5ccccc5)cc4)c4cccc5sc6c7ccccc7c(-c7cccc8ccccc78)cc6c45)cc3)cc2)cc1]

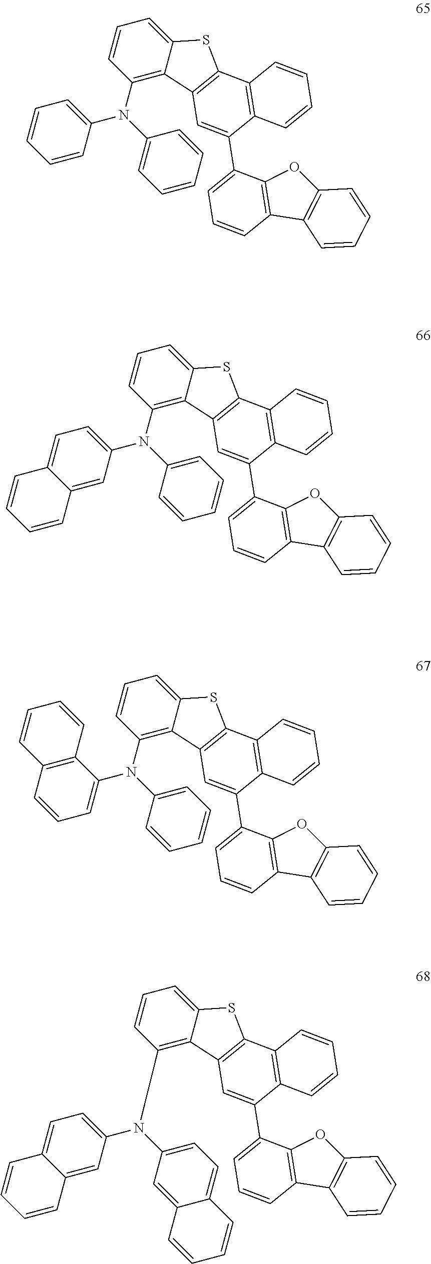 [SMILES: c1ccc(N(c2ccccc2)c2cccc3sc4c5ccccc5c(-c5cccc6c5oc5ccccc56)cc4c23)cc1 ; c1ccc(N(c2ccc3ccccc3c2)c2cccc3sc4c5ccccc5c(-c5cccc6c5oc5ccccc56)cc4c23)cc1 ; c1ccc(N(c2cccc3ccccc23)c2cccc3sc4c5ccccc5c(-c5cccc6c5oc5ccccc56)cc4c23)cc1 ; c1ccc2cc(N(c3ccc4ccccc4c3)c3cccc4sc5c6ccccc6c(-c6cccc7c6oc6ccccc67)cc5c34)ccc2c1]

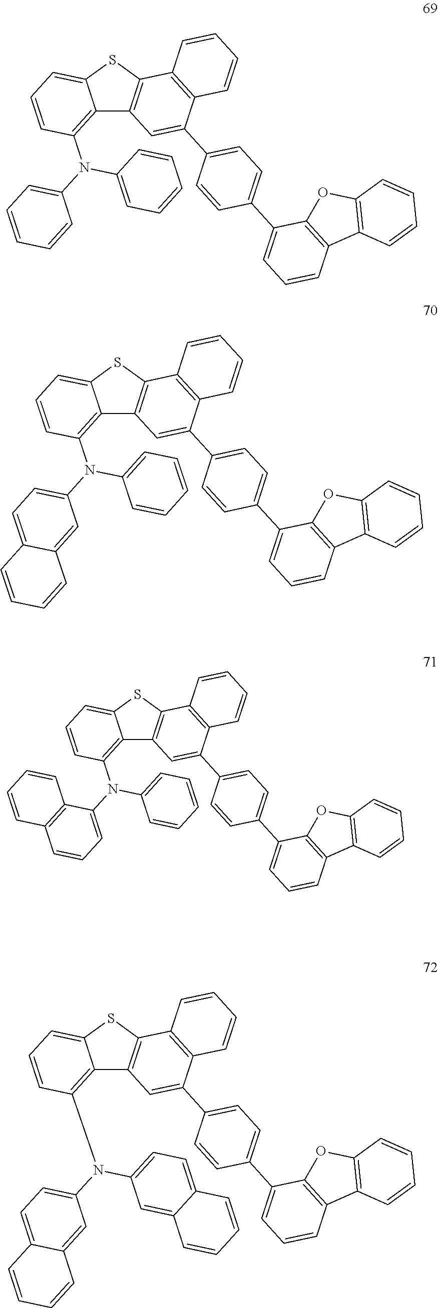 [SMILES: c1ccc(N(c2ccccc2)c2cccc3sc4c5ccccc5c(-c5ccc(-c6cccc7c6oc6ccccc67)cc5)cc4c23)cc1 ; c1ccc(N(c2ccc3ccccc3c2)c2cccc3sc4c5ccccc5c(-c5ccc(-c6cccc7c6oc6ccccc67)cc5)cc4c23)cc1 ; c1ccc(N(c2cccc3ccccc23)c2cccc3sc4c5ccccc5c(-c5ccc(-c6cccc7c6oc6ccccc67)cc5)cc4c23)cc1 ; c1ccc2cc(N(c3ccc4ccccc4c3)c3cccc4sc5c6ccccc6c(-c6ccc(-c7cccc8c7oc7ccccc78)cc6)cc5c34)ccc2c1]

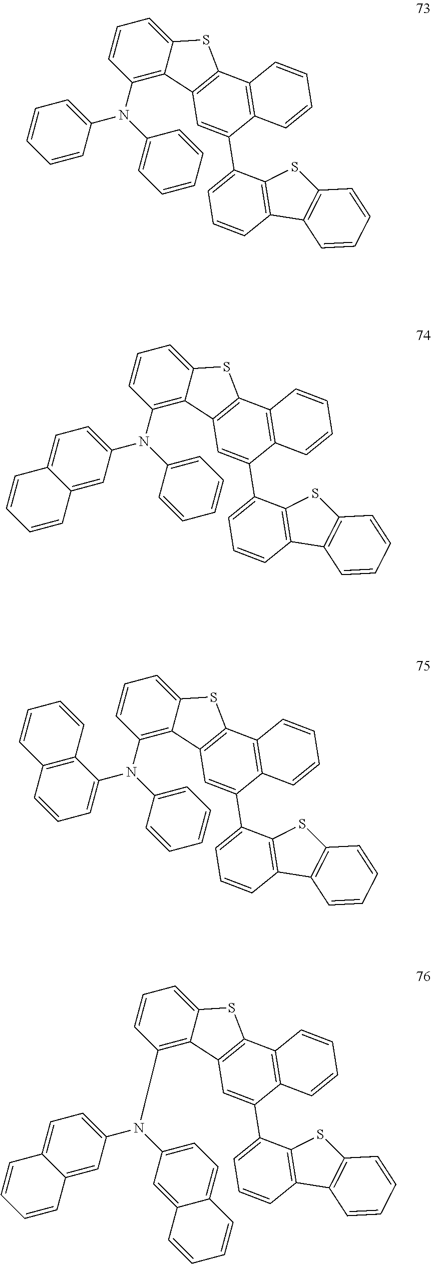 [SMILES: c1ccc(N(c2ccccc2)c2cccc3sc4c5ccccc5c(-c5cccc6c5sc5ccccc56)cc4c23)cc1 ; c1ccc(N(c2ccc3ccccc3c2)c2cccc3sc4c5ccccc5c(-c5cccc6c5sc5ccccc56)cc4c23)cc1 ; c1ccc(N(c2cccc3ccccc23)c2cccc3sc4c5ccccc5c(-c5cccc6c5sc5ccccc56)cc4c23)cc1 ; c1ccc2cc(N(c3ccc4ccccc4c3)c3cccc4sc5c6ccccc6c(-c6cccc7c6sc6ccccc67)cc5c34)ccc2c1]

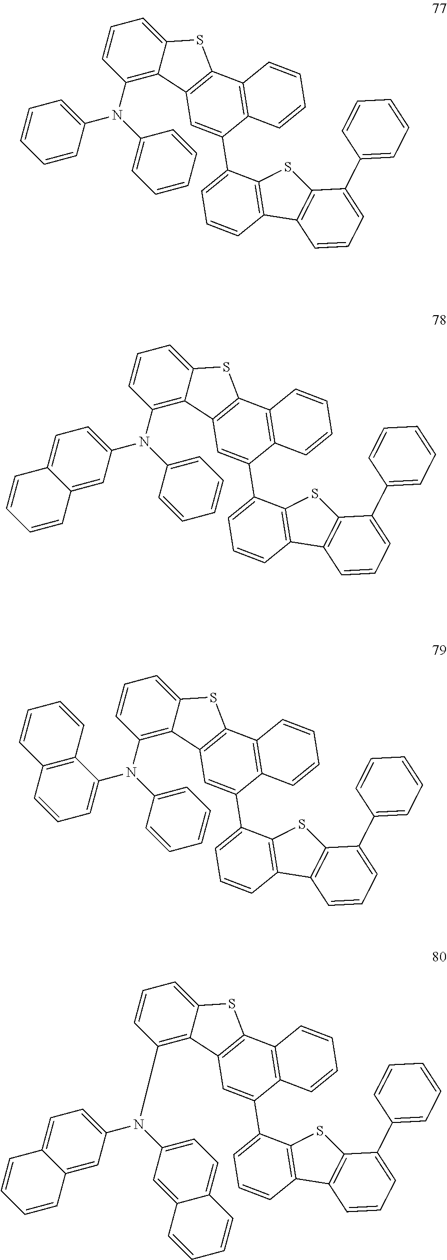 [SMILES: c1ccc(-c2cccc3c2sc2c(-c4cc5c(sc6cccc(N(c7ccccc7)c7ccccc7)c65)c5ccccc45)cccc23)cc1 ; c1ccc(-c2cccc3c2sc2c(-c4cc5c(sc6cccc(N(c7ccccc7)c7ccc8ccccc8c7)c65)c5ccccc45)cccc23)cc1 ; c1ccc(-c2cccc3c2sc2c(-c4cc5c(sc6cccc(N(c7ccccc7)c7cccc8ccccc78)c65)c5ccccc45)cccc23)cc1 ; c1ccc(-c2cccc3c2sc2c(-c4cc5c(sc6cccc(N(c7ccc8ccccc8c7)c7ccc8ccccc8c7)c65)c5ccccc45)cccc23)cc1]

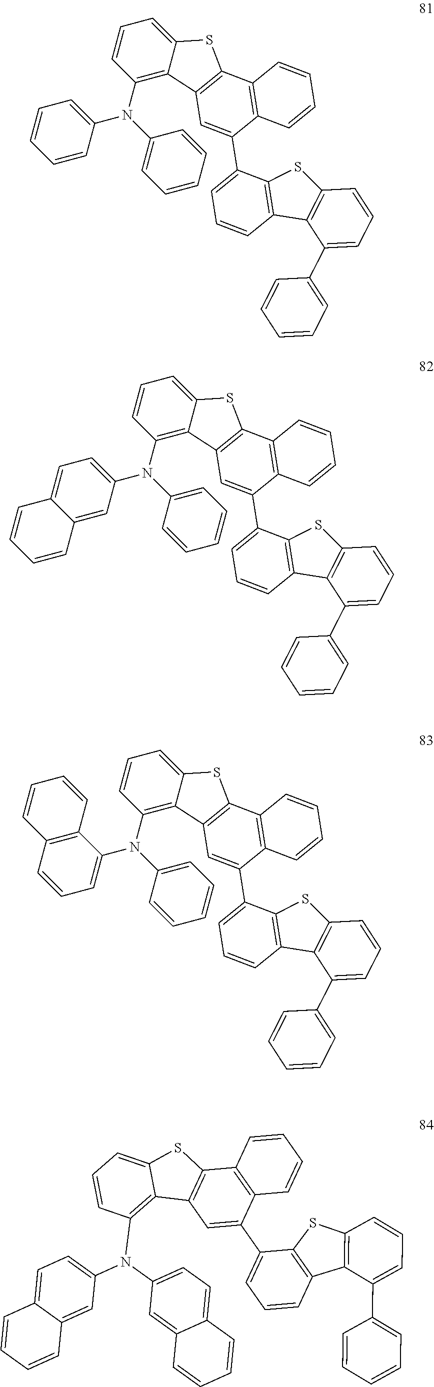 [SMILES: c1ccc(-c2cccc3sc4c(-c5cc6c(sc7cccc(N(c8ccccc8)c8ccccc8)c76)c6ccccc56)cccc4c23)cc1 ; c1ccc(-c2cccc3sc4c(-c5cc6c(sc7cccc(N(c8ccccc8)c8ccc9ccccc9c8)c76)c6ccccc56)cccc4c23)cc1 ; c1ccc(-c2cccc3sc4c(-c5cc6c(sc7cccc(N(c8ccccc8)c8cccc9ccccc89)c76)c6ccccc56)cccc4c23)cc1 ; c1ccc(-c2cccc3sc4c(-c5cc6c(sc7cccc(N(c8ccc9ccccc9c8)c8ccc9ccccc9c8)c76)c6ccccc56)cccc4c23)cc1]

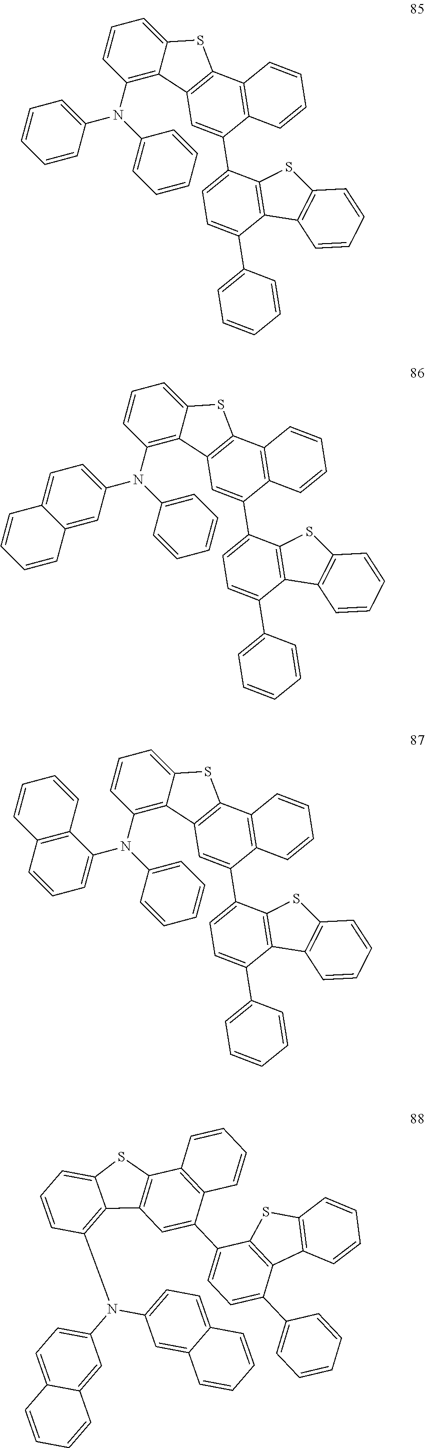 [SMILES: c1ccc(-c2ccc(-c3cc4c(sc5cccc(N(c6ccccc6)c6ccccc6)c54)c4ccccc34)c3sc4ccccc4c23)cc1 ; c1ccc(-c2ccc(-c3cc4c(sc5cccc(N(c6ccccc6)c6ccc7ccccc7c6)c54)c4ccccc34)c3sc4ccccc4c23)cc1 ; c1ccc(-c2ccc(-c3cc4c(sc5cccc(N(c6ccccc6)c6cccc7ccccc67)c54)c4ccccc34)c3sc4ccccc4c23)cc1 ; c1ccc(-c2ccc(-c3cc4c(sc5cccc(N(c6ccc7ccccc7c6)c6ccc7ccccc7c6)c54)c4ccccc34)c3sc4ccccc4c23)cc1]

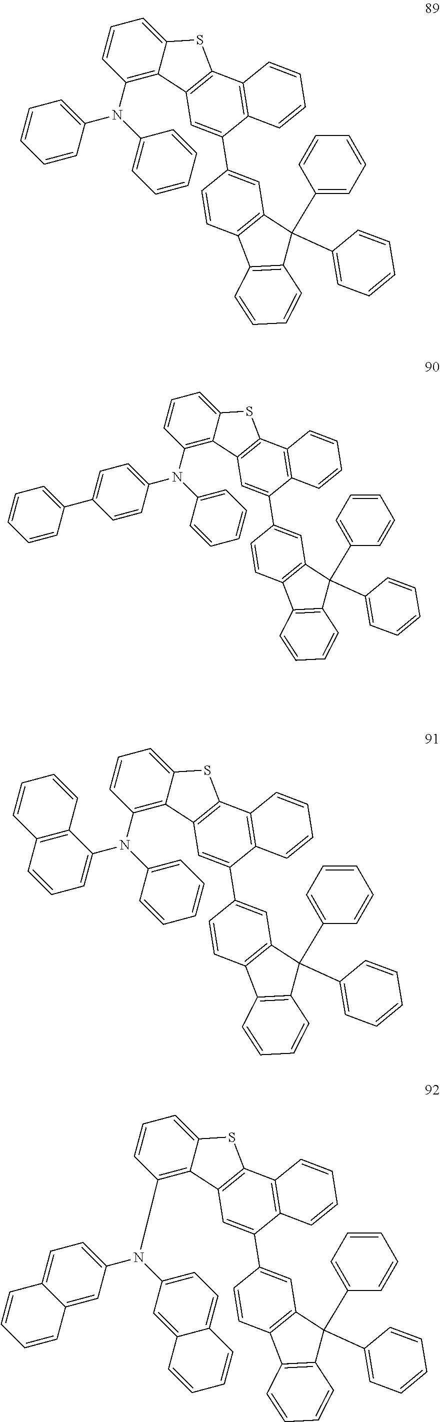 [SMILES: c1ccc(N(c2ccccc2)c2cccc3sc4c5ccccc5c(-c5ccc6c(c5)C(c5ccccc5)(c5ccccc5)c5ccccc5-6)cc4c23)cc1 ; c1ccc(-c2ccc(N(c3ccccc3)c3cccc4sc5c6ccccc6c(-c6ccc7c(c6)C(c6ccccc6)(c6ccccc6)c6ccccc6-7)cc5c34)cc2)cc1 ; c1ccc(N(c2cccc3ccccc23)c2cccc3sc4c5ccccc5c(-c5ccc6c(c5)C(c5ccccc5)(c5ccccc5)c5ccccc5-6)cc4c23)cc1 ; c1ccc(C2(c3ccccc3)c3ccccc3-c3ccc(-c4cc5c(sc6cccc(N(c7ccc8ccccc8c7)c7ccc8ccccc8c7)c65)c5ccccc45)cc32)cc1]

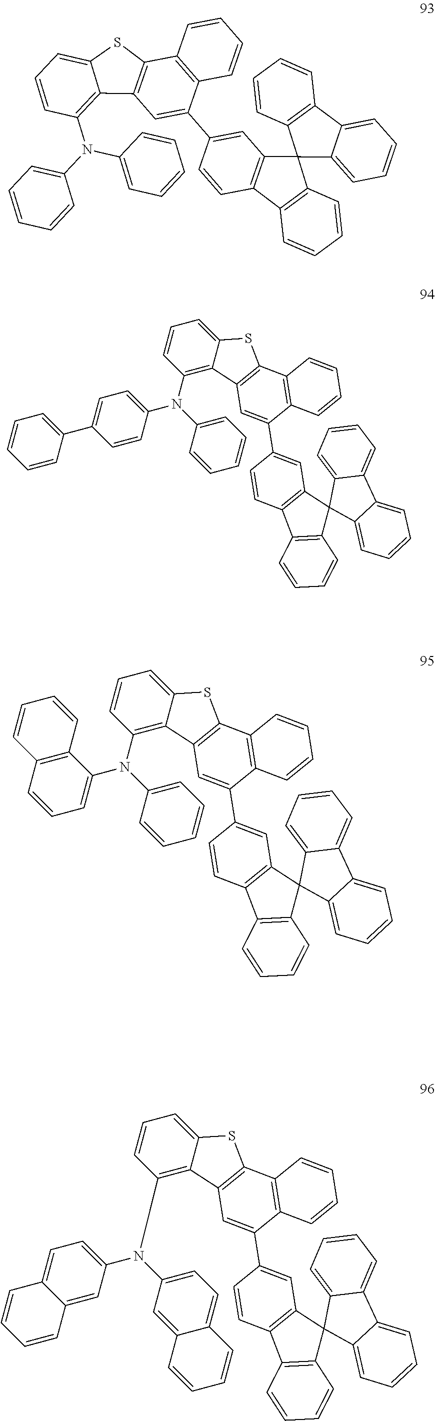 [SMILES: c1ccc(N(c2ccccc2)c2cccc3sc4c5ccccc5c(-c5ccc6c(c5)C5(c7ccccc7-c7ccccc75)c5ccccc5-6)cc4c23)cc1 ; c1ccc(-c2ccc(N(c3ccccc3)c3cccc4sc5c6ccccc6c(-c6ccc7c(c6)C6(c8ccccc8-c8ccccc86)c6ccccc6-7)cc5c34)cc2)cc1 ; c1ccc(N(c2cccc3ccccc23)c2cccc3sc4c5ccccc5c(-c5ccc6c(c5)C5(c7ccccc7-c7ccccc75)c5ccccc5-6)cc4c23)cc1 ; c1ccc2c(c1)-c1ccccc1C21c2ccccc2-c2ccc(-c3cc4c(sc5cccc(N(c6ccc7ccccc7c6)c6ccc7ccccc7c6)c54)c4ccccc34)cc21]

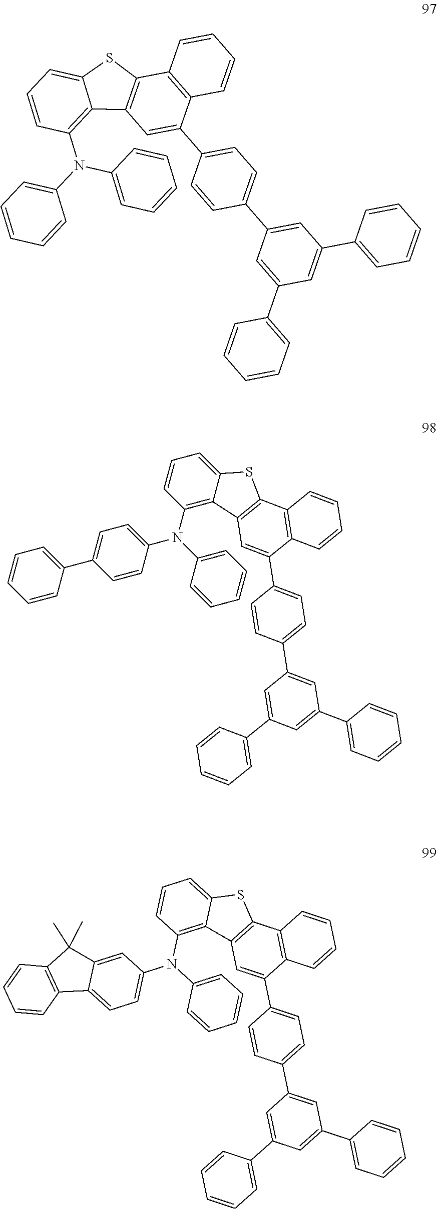 [SMILES: c1ccc(-c2cc(-c3ccccc3)cc(-c3ccc(-c4cc5c(sc6cccc(N(c7ccccc7)c7ccccc7)c65)c5ccccc45)cc3)c2)cc1 ; c1ccc(-c2ccc(N(c3ccccc3)c3cccc4sc5c6ccccc6c(-c6ccc(-c7cc(-c8ccccc8)cc(-c8ccccc8)c7)cc6)cc5c34)cc2)cc1 ; CC1(C)c2ccccc2-c2ccc(N(c3ccccc3)c3cccc4sc5c6ccccc6c(-c6ccc(-c7cc(-c8ccccc8)cc(-c8ccccc8)c7)cc6)cc5c34)cc21]

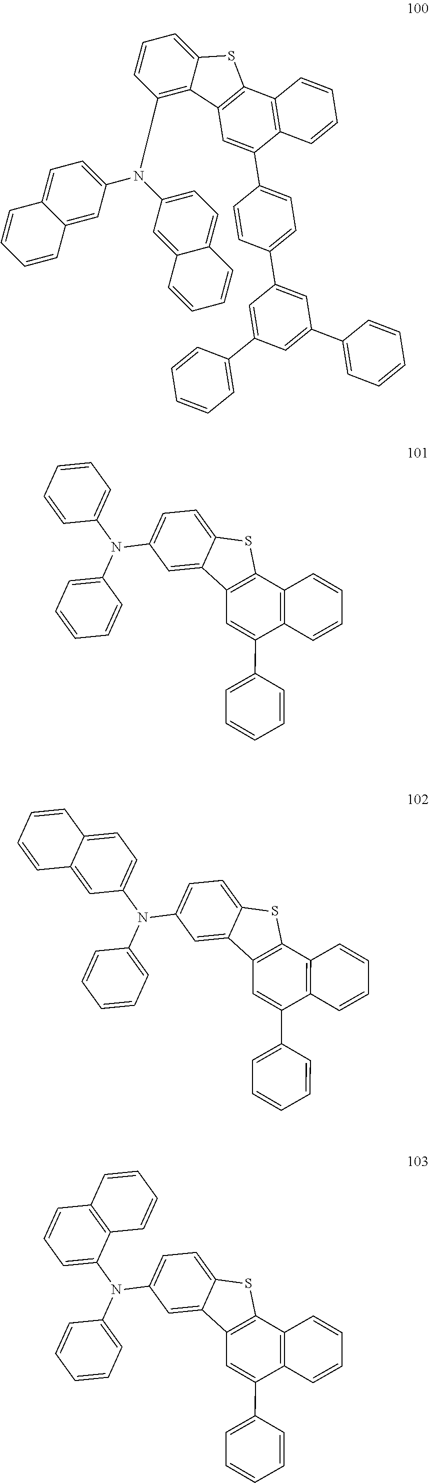 [SMILES: c1ccc(-c2cc(-c3ccccc3)cc(-c3ccc(-c4cc5c(sc6cccc(N(c7ccc8ccccc8c7)c7ccc8ccccc8c7)c65)c5ccccc45)cc3)c2)cc1 ; c1ccc(-c2cc3c4cc(N(c5ccccc5)c5ccccc5)ccc4sc3c3ccccc23)cc1 ; c1ccc(-c2cc3c4cc(N(c5ccccc5)c5ccc6ccccc6c5)ccc4sc3c3ccccc23)cc1 ; c1ccc(-c2cc3c4cc(N(c5ccccc5)c5cccc6ccccc56)ccc4sc3c3ccccc23)cc1]

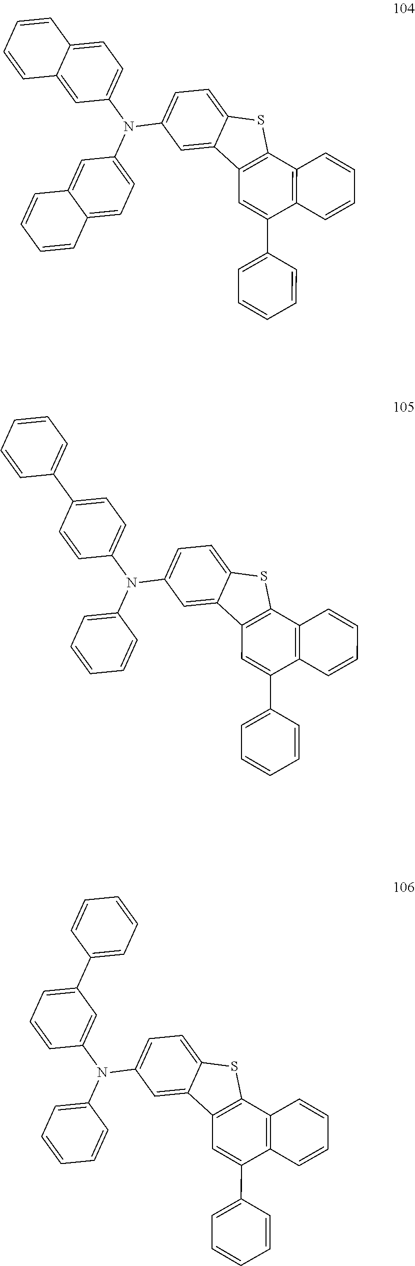 [SMILES: c1ccc(-c2cc3c4cc(N(c5ccc6ccccc6c5)c5ccc6ccccc6c5)ccc4sc3c3ccccc23)cc1 ; c1ccc(-c2ccc(N(c3ccccc3)c3ccc4sc5c6ccccc6c(-c6ccccc6)cc5c4c3)cc2)cc1 ; c1ccc(-c2cccc(N(c3ccccc3)c3ccc4sc5c6ccccc6c(-c6ccccc6)cc5c4c3)c2)cc1]

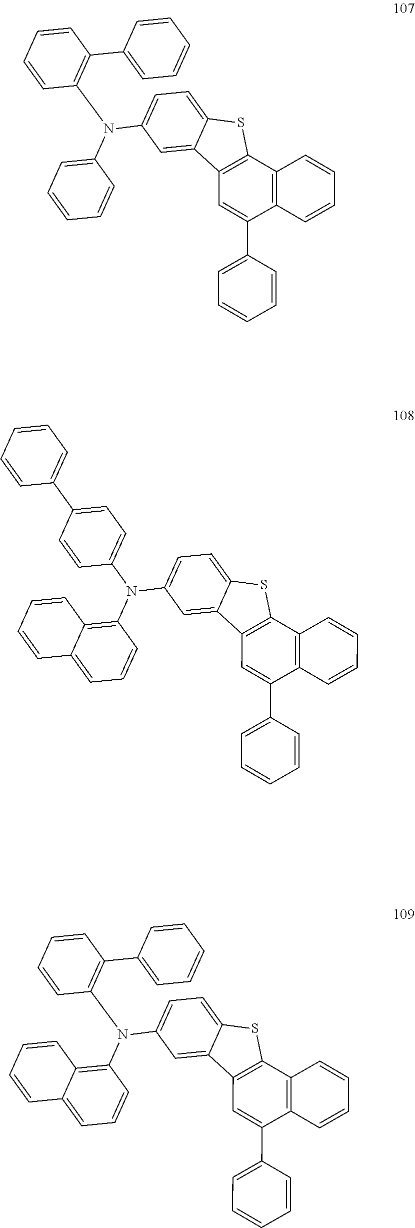 [SMILES: c1ccc(-c2ccccc2N(c2ccccc2)c2ccc3sc4c5ccccc5c(-c5ccccc5)cc4c3c2)cc1 ; c1ccc(-c2ccc(N(c3ccc4sc5c6ccccc6c(-c6ccccc6)cc5c4c3)c3cccc4ccccc34)cc2)cc1 ; c1ccc(-c2ccccc2N(c2ccc3sc4c5ccccc5c(-c5ccccc5)cc4c3c2)c2cccc3ccccc23)cc1]

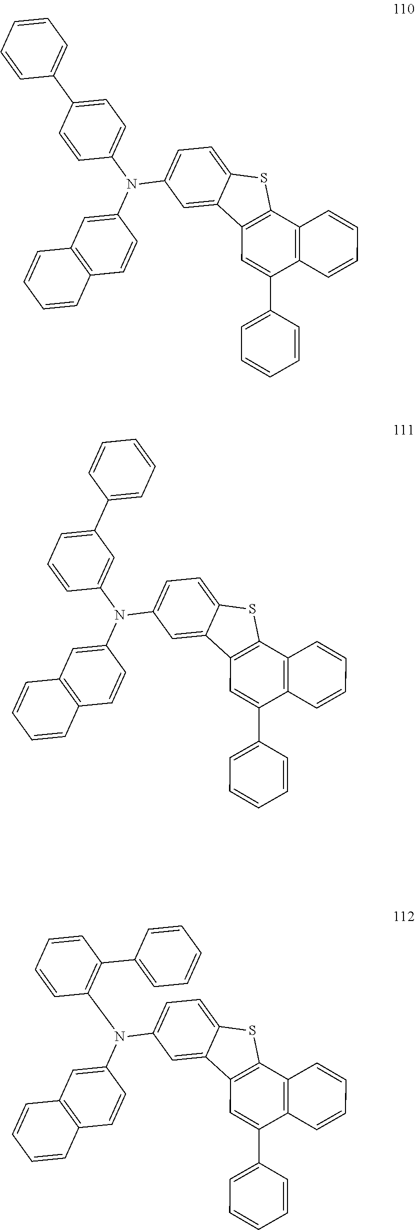 [SMILES: c1ccc(-c2ccc(N(c3ccc4ccccc4c3)c3ccc4sc5c6ccccc6c(-c6ccccc6)cc5c4c3)cc2)cc1 ; c1ccc(-c2cccc(N(c3ccc4ccccc4c3)c3ccc4sc5c6ccccc6c(-c6ccccc6)cc5c4c3)c2)cc1 ; c1ccc(-c2ccccc2N(c2ccc3ccccc3c2)c2ccc3sc4c5ccccc5c(-c5ccccc5)cc4c3c2)cc1]

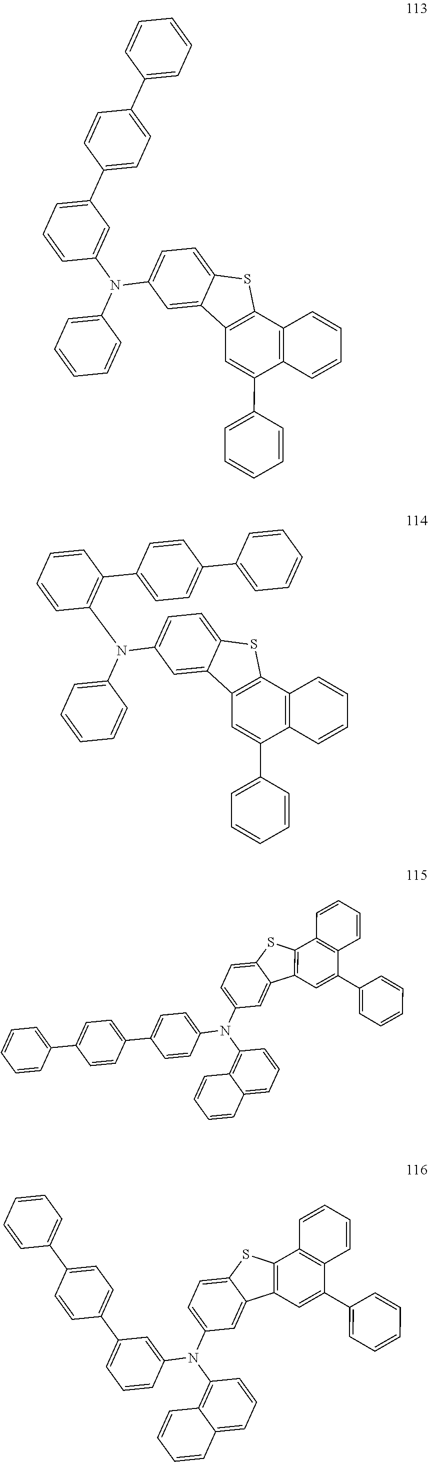 [SMILES: c1ccc(-c2ccc(-c3cccc(N(c4ccccc4)c4ccc5sc6c7ccccc7c(-c7ccccc7)cc6c5c4)c3)cc2)cc1 ; c1ccc(-c2ccc(-c3ccccc3N(c3ccccc3)c3ccc4sc5c6ccccc6c(-c6ccccc6)cc5c4c3)cc2)cc1 ; c1ccc(-c2ccc(-c3ccc(N(c4ccc5sc6c7ccccc7c(-c7ccccc7)cc6c5c4)c4cccc5ccccc45)cc3)cc2)cc1 ; c1ccc(-c2ccc(-c3cccc(N(c4ccc5sc6c7ccccc7c(-c7ccccc7)cc6c5c4)c4cccc5ccccc45)c3)cc2)cc1]

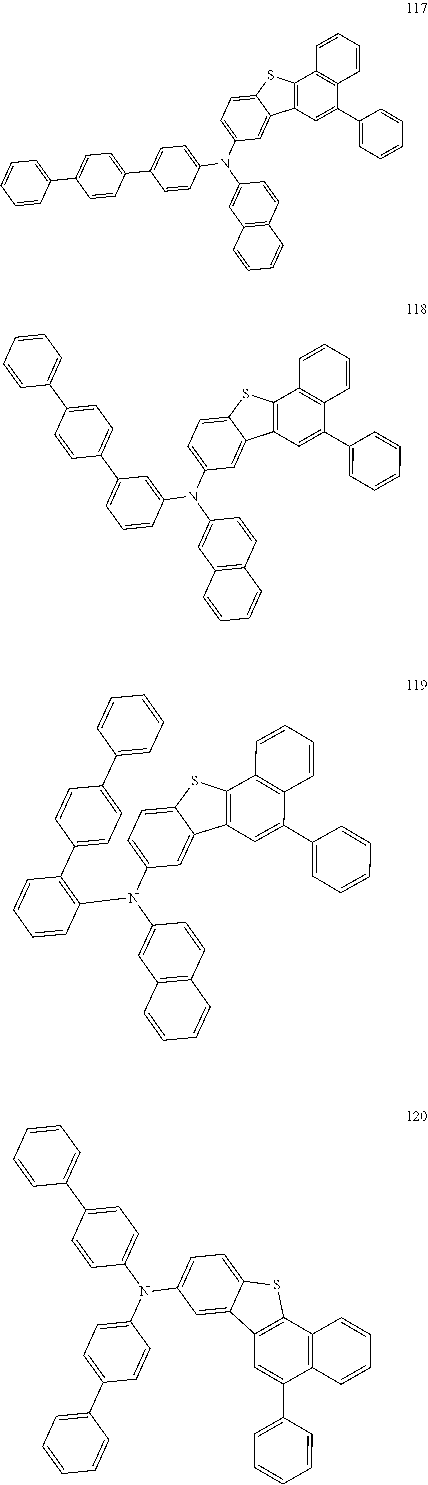 [SMILES: c1ccc(-c2ccc(-c3ccc(N(c4ccc5ccccc5c4)c4ccc5sc6c7ccccc7c(-c7ccccc7)cc6c5c4)cc3)cc2)cc1 ; c1ccc(-c2ccc(-c3cccc(N(c4ccc5ccccc5c4)c4ccc5sc6c7ccccc7c(-c7ccccc7)cc6c5c4)c3)cc2)cc1 ; c1ccc(-c2ccc(-c3ccccc3N(c3ccc4ccccc4c3)c3ccc4sc5c6ccccc6c(-c6ccccc6)cc5c4c3)cc2)cc1 ; c1ccc(-c2ccc(N(c3ccc(-c4ccccc4)cc3)c3ccc4sc5c6ccccc6c(-c6ccccc6)cc5c4c3)cc2)cc1]

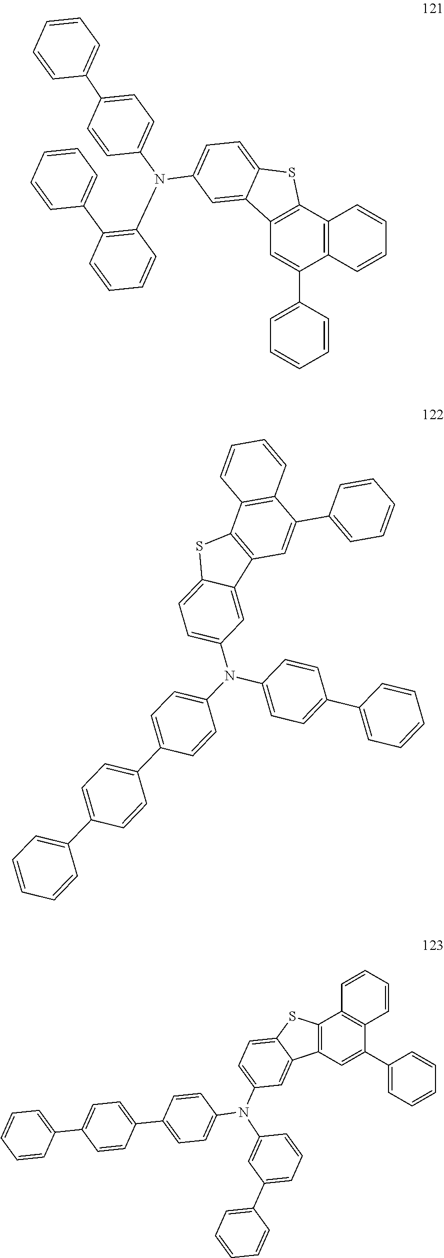 [SMILES: c1ccc(-c2ccc(N(c3ccc4sc5c6ccccc6c(-c6ccccc6)cc5c4c3)c3ccccc3-c3ccccc3)cc2)cc1 ; c1ccc(-c2ccc(-c3ccc(N(c4ccc(-c5ccccc5)cc4)c4ccc5sc6c7ccccc7c(-c7ccccc7)cc6c5c4)cc3)cc2)cc1 ; c1ccc(-c2ccc(-c3ccc(N(c4cccc(-c5ccccc5)c4)c4ccc5sc6c7ccccc7c(-c7ccccc7)cc6c5c4)cc3)cc2)cc1]

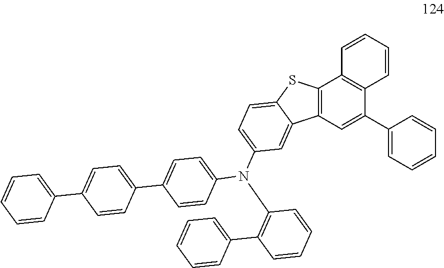 [SMILES: c1ccc(-c2ccc(-c3ccc(N(c4ccc5sc6c7ccccc7c(-c7ccccc7)cc6c5c4)c4ccccc4-c4ccccc4)cc3)cc2)cc1]

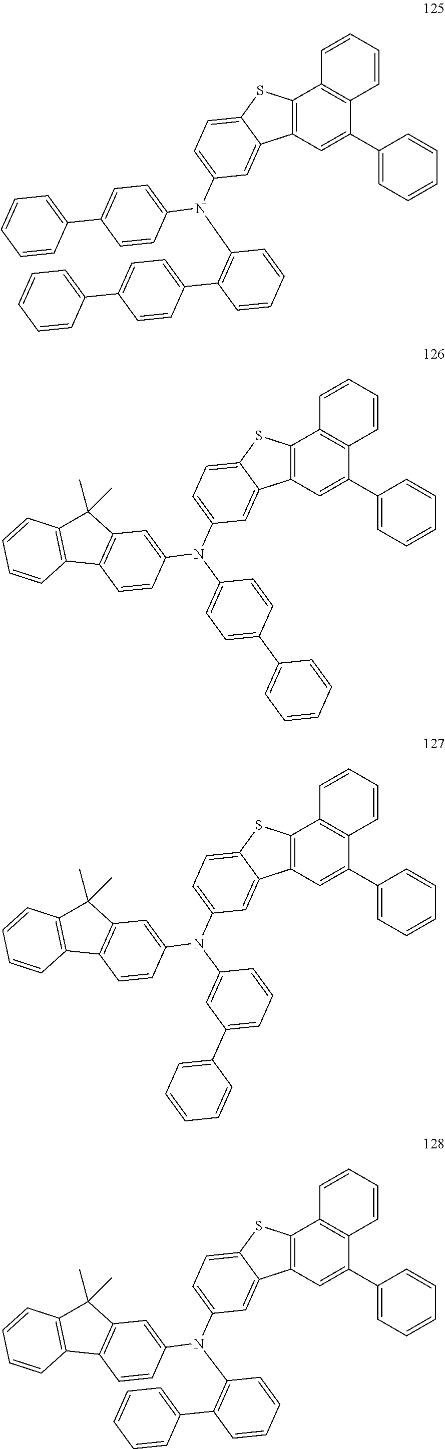 [SMILES: c1ccc(-c2ccc(-c3ccccc3N(c3ccc(-c4ccccc4)cc3)c3ccc4sc5c6ccccc6c(-c6ccccc6)cc5c4c3)cc2)cc1 ; CC1(C)c2ccccc2-c2ccc(N(c3ccc(-c4ccccc4)cc3)c3ccc4sc5c6ccccc6c(-c6ccccc6)cc5c4c3)cc21 ; CC1(C)c2ccccc2-c2ccc(N(c3cccc(-c4ccccc4)c3)c3ccc4sc5c6ccccc6c(-c6ccccc6)cc5c4c3)cc21 ; CC1(C)c2ccccc2-c2ccc(N(c3ccc4sc5c6ccccc6c(-c6ccccc6)cc5c4c3)c3ccccc3-c3ccccc3)cc21]

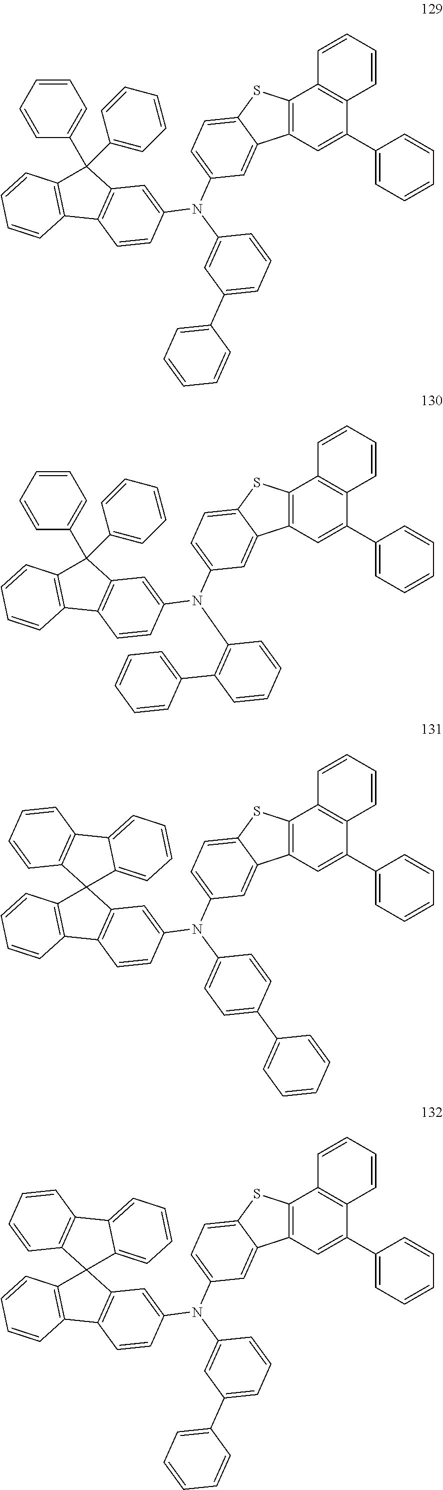 [SMILES: c1ccc(-c2cccc(N(c3ccc4c(c3)C(c3ccccc3)(c3ccccc3)c3ccccc3-4)c3ccc4sc5c6ccccc6c(-c6ccccc6)cc5c4c3)c2)cc1 ; c1ccc(-c2ccccc2N(c2ccc3c(c2)C(c2ccccc2)(c2ccccc2)c2ccccc2-3)c2ccc3sc4c5ccccc5c(-c5ccccc5)cc4c3c2)cc1 ; c1ccc(-c2ccc(N(c3ccc4c(c3)C3(c5ccccc5-c5ccccc53)c3ccccc3-4)c3ccc4sc5c6ccccc6c(-c6ccccc6)cc5c4c3)cc2)cc1 ; c1ccc(-c2cccc(N(c3ccc4c(c3)C3(c5ccccc5-c5ccccc53)c3ccccc3-4)c3ccc4sc5c6ccccc6c(-c6ccccc6)cc5c4c3)c2)cc1]

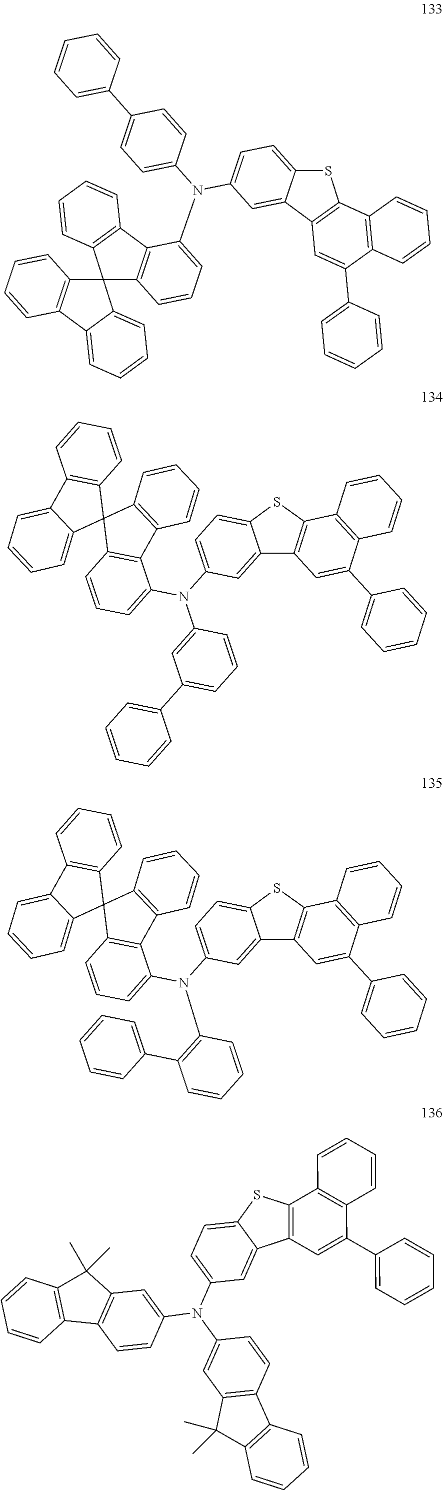 [SMILES: c1ccc(-c2ccc(N(c3ccc4sc5c6ccccc6c(-c6ccccc6)cc5c4c3)c3cccc4c3-c3ccccc3C43c4ccccc4-c4ccccc43)cc2)cc1 ; c1ccc(-c2cccc(N(c3ccc4sc5c6ccccc6c(-c6ccccc6)cc5c4c3)c3cccc4c3-c3ccccc3C43c4ccccc4-c4ccccc43)c2)cc1 ; c1ccc(-c2ccccc2N(c2ccc3sc4c5ccccc5c(-c5ccccc5)cc4c3c2)c2cccc3c2-c2ccccc2C32c3ccccc3-c3ccccc32)cc1 ; CC1(C)c2ccccc2-c2ccc(N(c3ccc4c(c3)C(C)(C)c3ccccc3-4)c3ccc4sc5c6ccccc6c(-c6ccccc6)cc5c4c3)cc21]

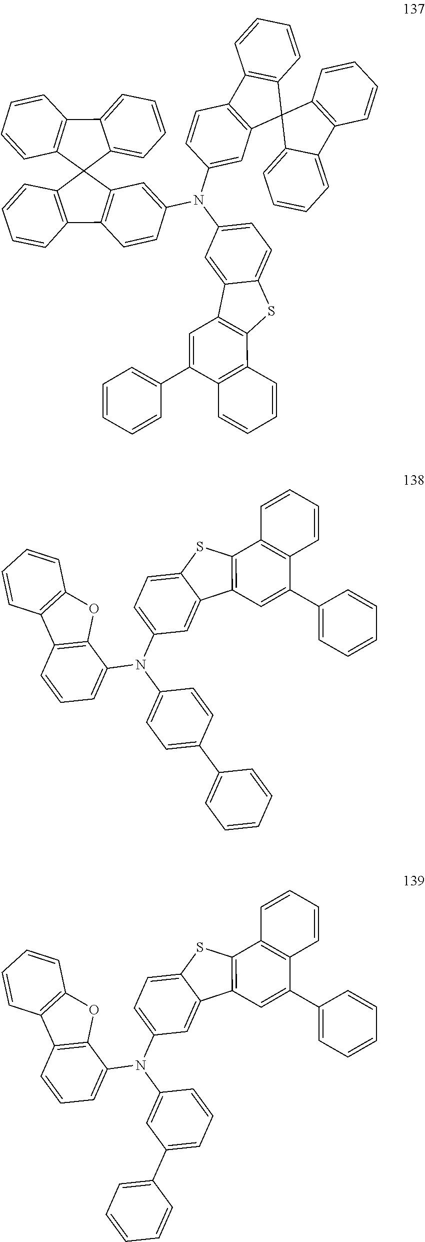 [SMILES: c1ccc(-c2cc3c4cc(N(c5ccc6c(c5)C5(c7ccccc7-c7ccccc75)c5ccccc5-6)c5ccc6c(c5)C5(c7ccccc7-c7ccccc75)c5ccccc5-6)ccc4sc3c3ccccc23)cc1 ; c1ccc(-c2ccc(N(c3ccc4sc5c6ccccc6c(-c6ccccc6)cc5c4c3)c3cccc4c3oc3ccccc34)cc2)cc1 ; c1ccc(-c2cccc(N(c3ccc4sc5c6ccccc6c(-c6ccccc6)cc5c4c3)c3cccc4c3oc3ccccc34)c2)cc1]

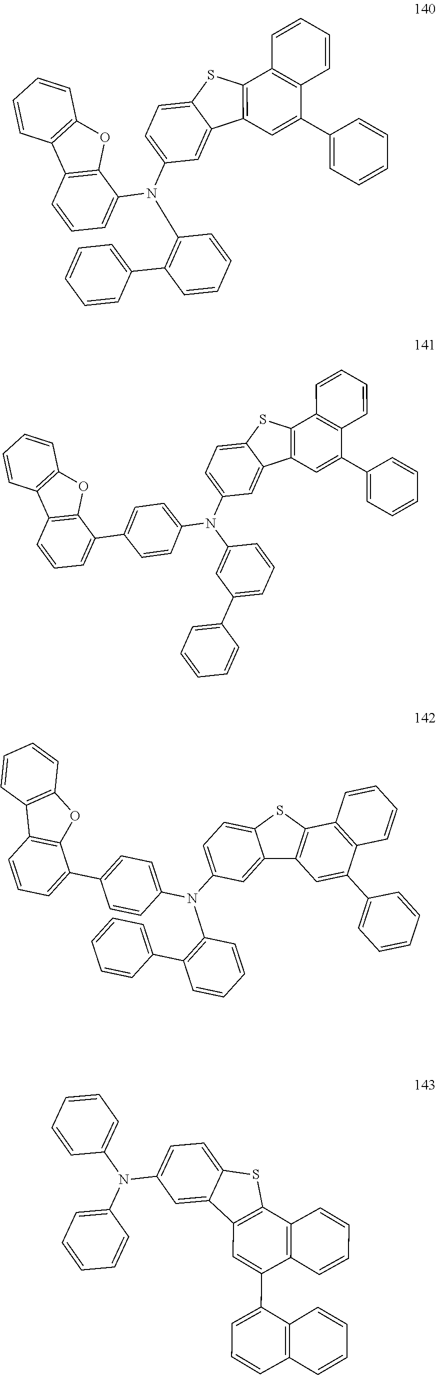 [SMILES: c1ccc(-c2ccccc2N(c2ccc3sc4c5ccccc5c(-c5ccccc5)cc4c3c2)c2cccc3c2oc2ccccc23)cc1 ; c1ccc(-c2cccc(N(c3ccc(-c4cccc5c4oc4ccccc45)cc3)c3ccc4sc5c6ccccc6c(-c6ccccc6)cc5c4c3)c2)cc1 ; c1ccc(-c2ccccc2N(c2ccc(-c3cccc4c3oc3ccccc34)cc2)c2ccc3sc4c5ccccc5c(-c5ccccc5)cc4c3c2)cc1 ; c1ccc(N(c2ccccc2)c2ccc3sc4c5ccccc5c(-c5cccc6ccccc56)cc4c3c2)cc1]

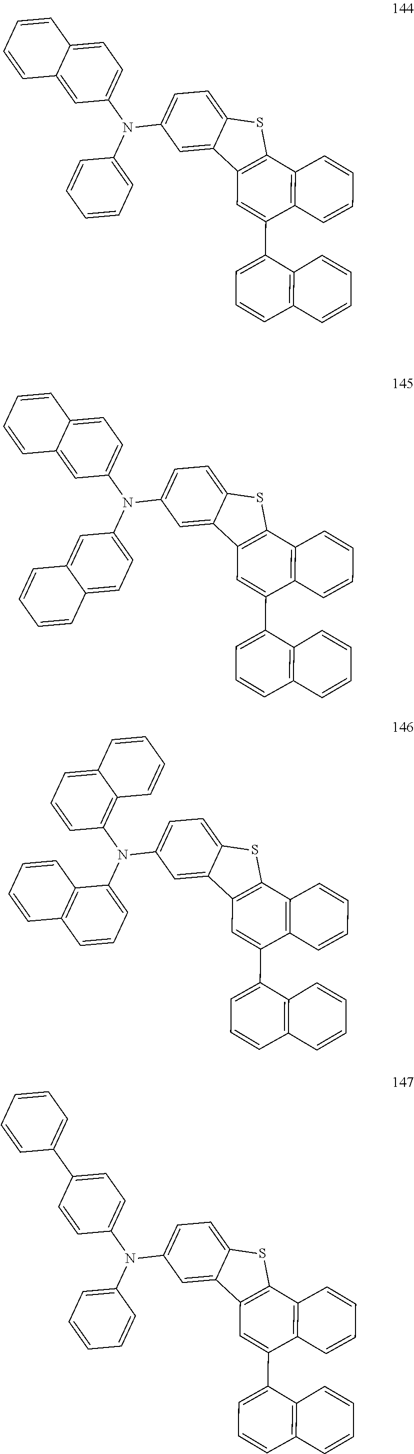 [SMILES: c1ccc(N(c2ccc3ccccc3c2)c2ccc3sc4c5ccccc5c(-c5cccc6ccccc56)cc4c3c2)cc1 ; c1ccc2cc(N(c3ccc4ccccc4c3)c3ccc4sc5c6ccccc6c(-c6cccc7ccccc67)cc5c4c3)ccc2c1 ; c1ccc2c(-c3cc4c5cc(N(c6cccc7ccccc67)c6cccc7ccccc67)ccc5sc4c4ccccc34)cccc2c1 ; c1ccc(-c2ccc(N(c3ccccc3)c3ccc4sc5c6ccccc6c(-c6cccc7ccccc67)cc5c4c3)cc2)cc1]

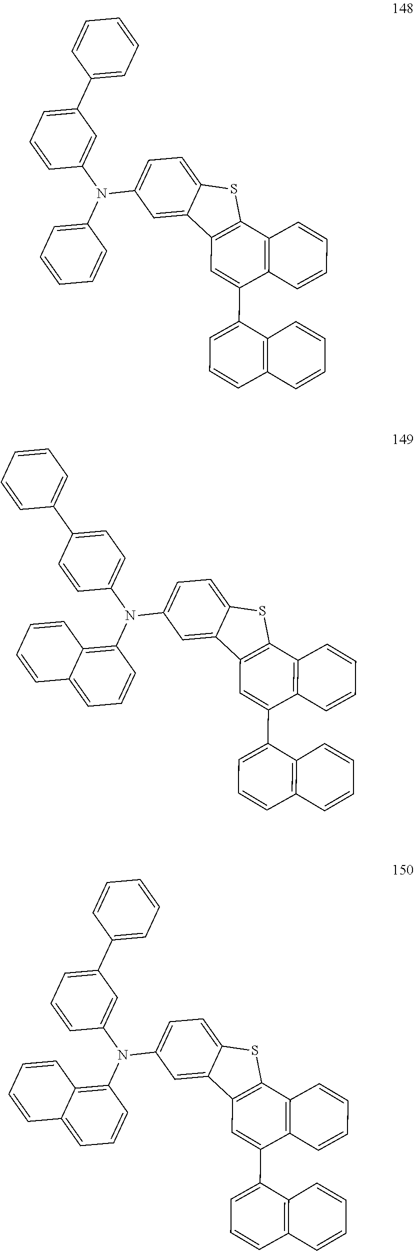 [SMILES: c1ccc(-c2cccc(N(c3ccccc3)c3ccc4sc5c6ccccc6c(-c6cccc7ccccc67)cc5c4c3)c2)cc1 ; c1ccc(-c2ccc(N(c3ccc4sc5c6ccccc6c(-c6cccc7ccccc67)cc5c4c3)c3cccc4ccccc34)cc2)cc1 ; c1ccc(-c2cccc(N(c3ccc4sc5c6ccccc6c(-c6cccc7ccccc67)cc5c4c3)c3cccc4ccccc34)c2)cc1]

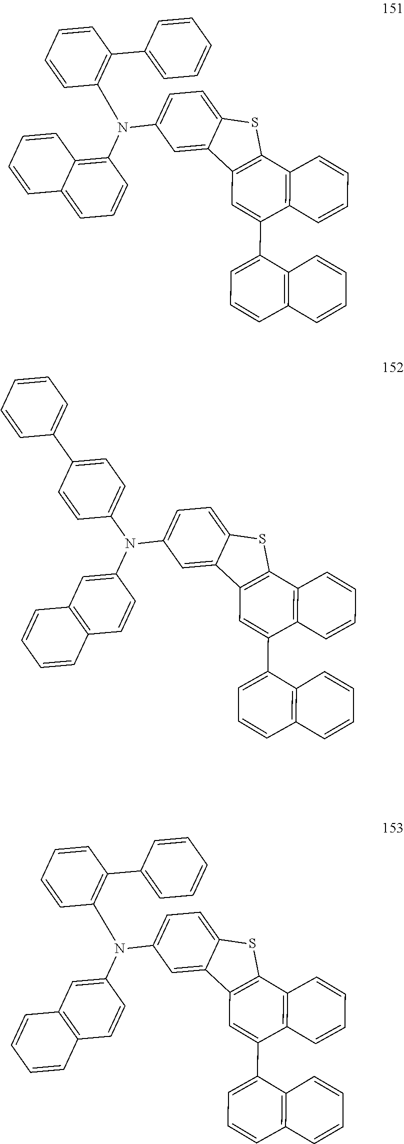 [SMILES: c1ccc(-c2ccccc2N(c2ccc3sc4c5ccccc5c(-c5cccc6ccccc56)cc4c3c2)c2cccc3ccccc23)cc1 ; c1ccc(-c2ccc(N(c3ccc4ccccc4c3)c3ccc4sc5c6ccccc6c(-c6cccc7ccccc67)cc5c4c3)cc2)cc1 ; c1ccc(-c2ccccc2N(c2ccc3ccccc3c2)c2ccc3sc4c5ccccc5c(-c5cccc6ccccc56)cc4c3c2)cc1]

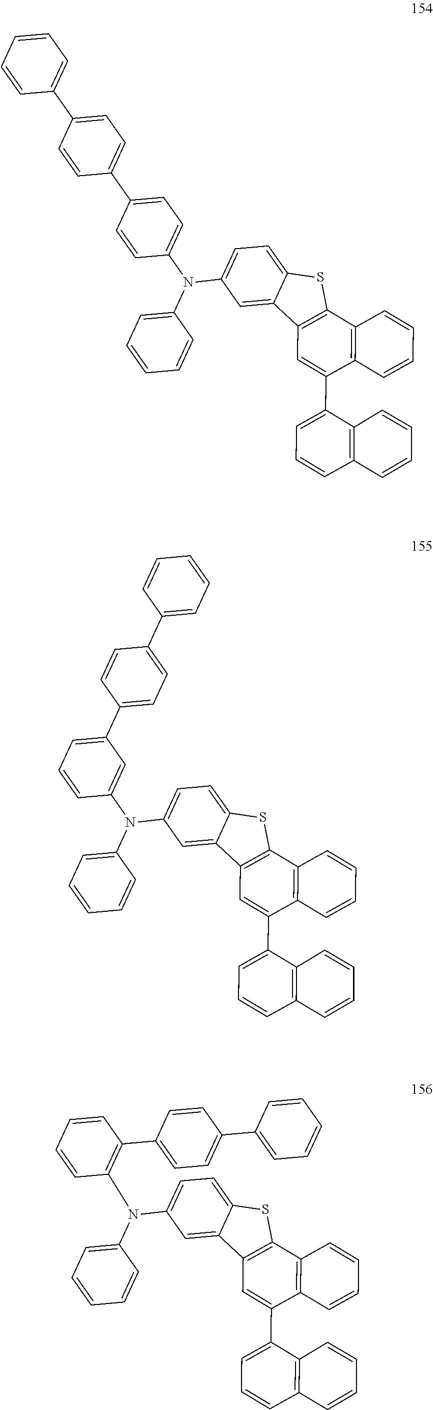 [SMILES: c1ccc(-c2ccc(-c3ccc(N(c4ccccc4)c4ccc5sc6c7ccccc7c(-c7cccc8ccccc78)cc6c5c4)cc3)cc2)cc1 ; c1ccc(-c2ccc(-c3cccc(N(c4ccccc4)c4ccc5sc6c7ccccc7c(-c7cccc8ccccc78)cc6c5c4)c3)cc2)cc1 ; c1ccc(-c2ccc(-c3ccccc3N(c3ccccc3)c3ccc4sc5c6ccccc6c(-c6cccc7ccccc67)cc5c4c3)cc2)cc1]

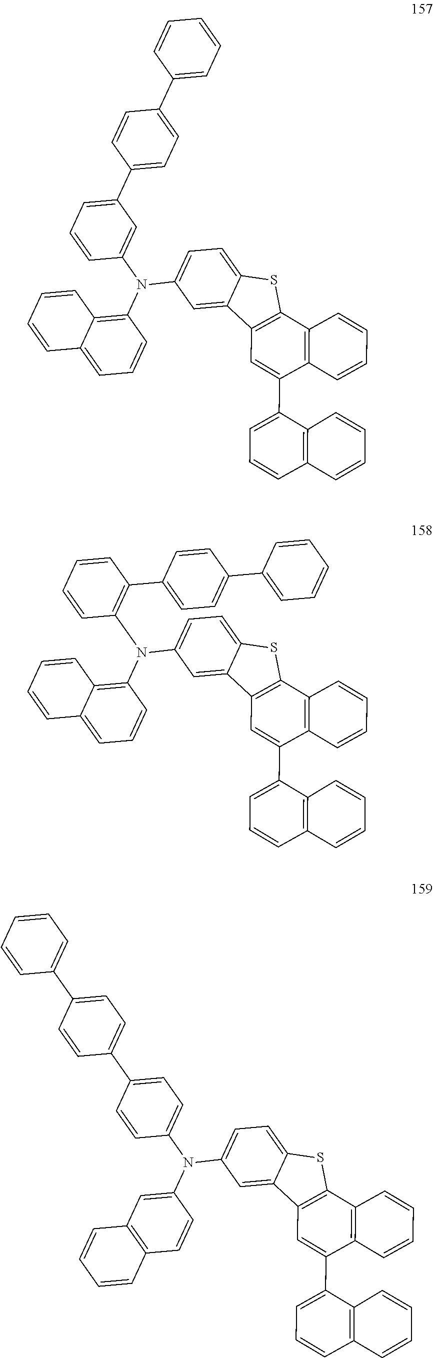 [SMILES: c1ccc(-c2ccc(-c3cccc(N(c4ccc5sc6c7ccccc7c(-c7cccc8ccccc78)cc6c5c4)c4cccc5ccccc45)c3)cc2)cc1 ; c1ccc(-c2ccc(-c3ccccc3N(c3ccc4sc5c6ccccc6c(-c6cccc7ccccc67)cc5c4c3)c3cccc4ccccc34)cc2)cc1 ; c1ccc(-c2ccc(-c3ccc(N(c4ccc5ccccc5c4)c4ccc5sc6c7ccccc7c(-c7cccc8ccccc78)cc6c5c4)cc3)cc2)cc1]

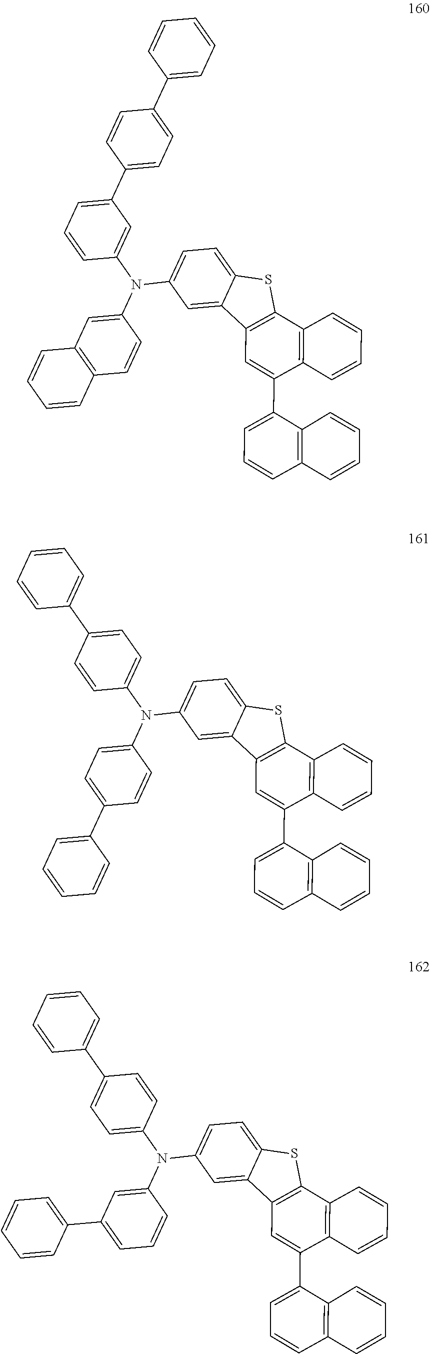 [SMILES: c1ccc(-c2ccc(-c3cccc(N(c4ccc5ccccc5c4)c4ccc5sc6c7ccccc7c(-c7cccc8ccccc78)cc6c5c4)c3)cc2)cc1 ; c1ccc(-c2ccc(N(c3ccc(-c4ccccc4)cc3)c3ccc4sc5c6ccccc6c(-c6cccc7ccccc67)cc5c4c3)cc2)cc1 ; c1ccc(-c2ccc(N(c3cccc(-c4ccccc4)c3)c3ccc4sc5c6ccccc6c(-c6cccc7ccccc67)cc5c4c3)cc2)cc1]

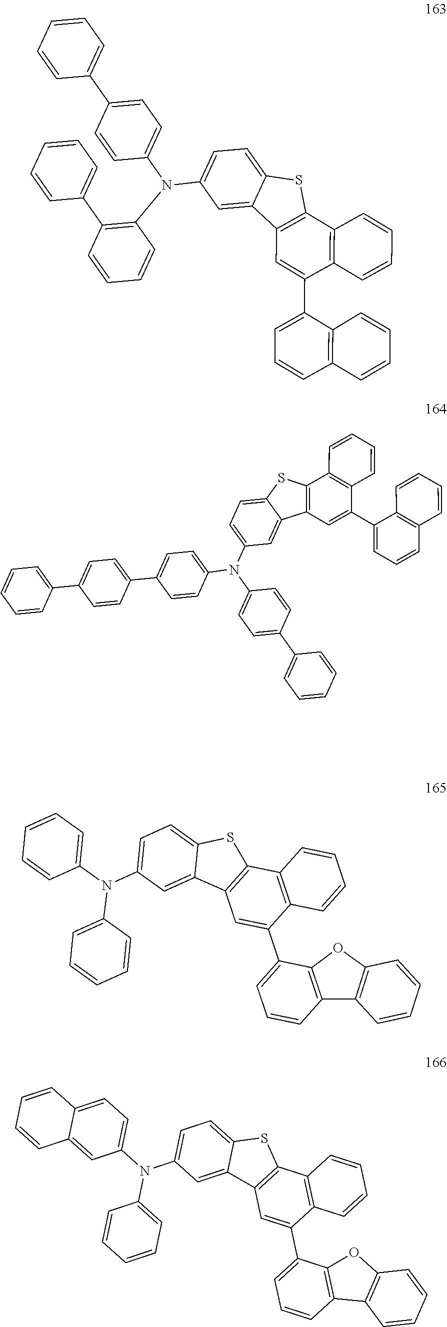 [SMILES: c1ccc(-c2ccc(N(c3ccc4sc5c6ccccc6c(-c6cccc7ccccc67)cc5c4c3)c3ccccc3-c3ccccc3)cc2)cc1 ; c1ccc(-c2ccc(-c3ccc(N(c4ccc(-c5ccccc5)cc4)c4ccc5sc6c7ccccc7c(-c7cccc8ccccc78)cc6c5c4)cc3)cc2)cc1 ; c1ccc(N(c2ccccc2)c2ccc3sc4c5ccccc5c(-c5cccc6c5oc5ccccc56)cc4c3c2)cc1 ; c1ccc(N(c2ccc3ccccc3c2)c2ccc3sc4c5ccccc5c(-c5cccc6c5oc5ccccc56)cc4c3c2)cc1]

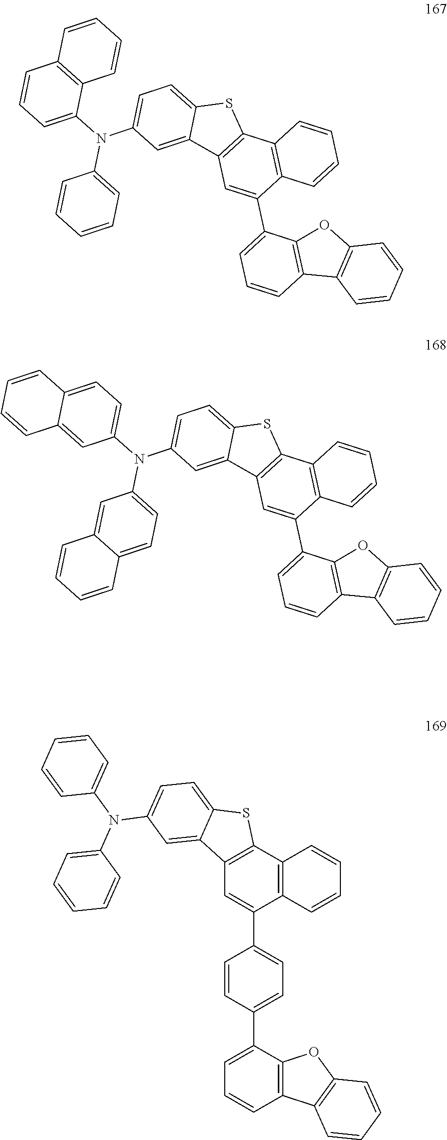 [SMILES: c1ccc(N(c2ccc3sc4c5ccccc5c(-c5cccc6c5oc5ccccc56)cc4c3c2)c2cccc3ccccc23)cc1 ; c1ccc2cc(N(c3ccc4ccccc4c3)c3ccc4sc5c6ccccc6c(-c6cccc7c6oc6ccccc67)cc5c4c3)ccc2c1 ; c1ccc(N(c2ccccc2)c2ccc3sc4c5ccccc5c(-c5ccc(-c6cccc7c6oc6ccccc67)cc5)cc4c3c2)cc1]

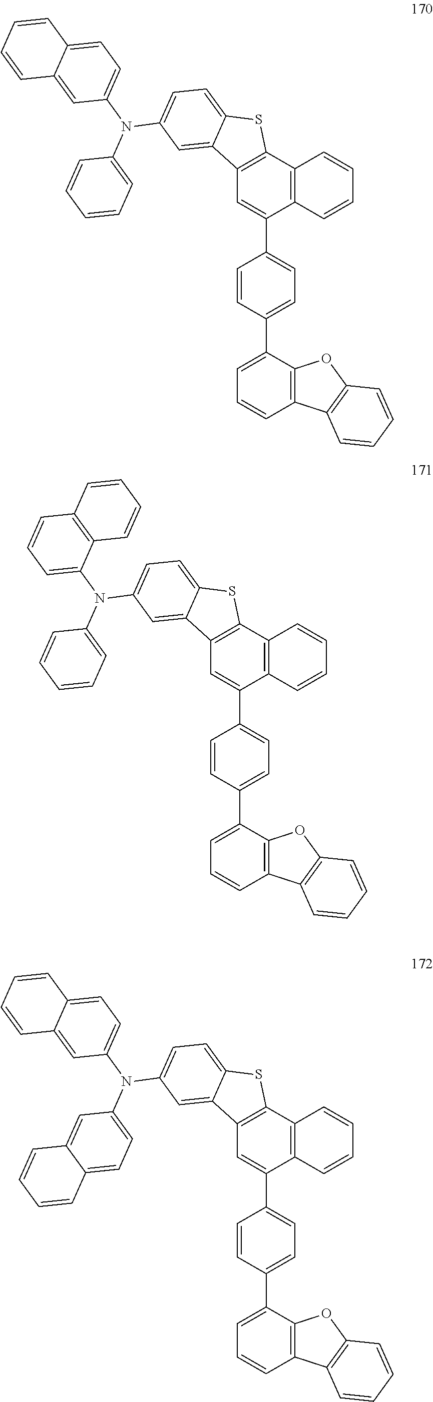 [SMILES: c1ccc(N(c2ccc3ccccc3c2)c2ccc3sc4c5ccccc5c(-c5ccc(-c6cccc7c6oc6ccccc67)cc5)cc4c3c2)cc1 ; c1ccc(N(c2ccc3sc4c5ccccc5c(-c5ccc(-c6cccc7c6oc6ccccc67)cc5)cc4c3c2)c2cccc3ccccc23)cc1 ; c1ccc2cc(N(c3ccc4ccccc4c3)c3ccc4sc5c6ccccc6c(-c6ccc(-c7cccc8c7oc7ccccc78)cc6)cc5c4c3)ccc2c1]

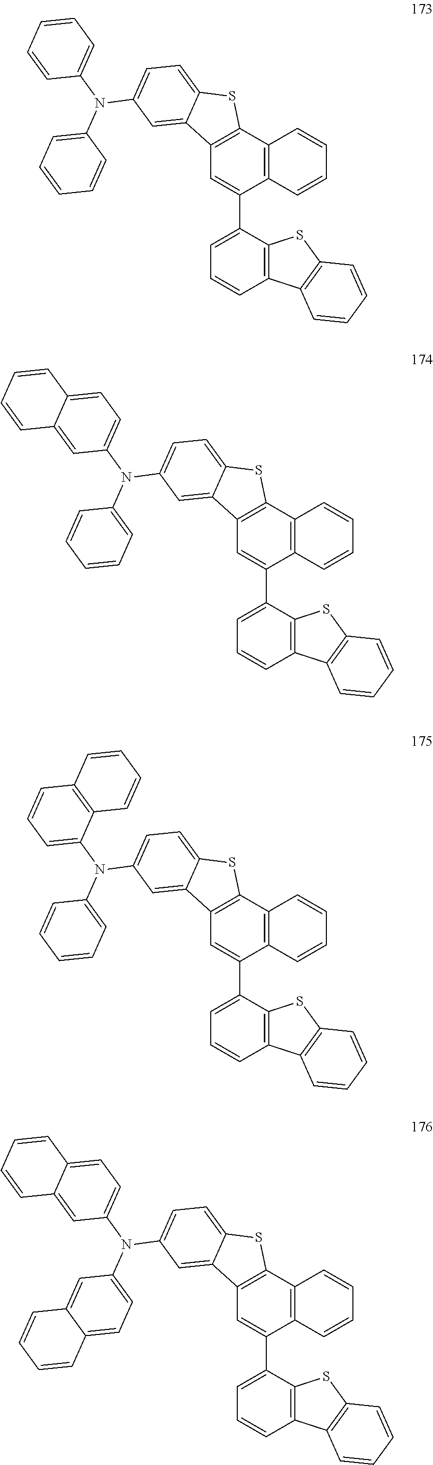 [SMILES: c1ccc(N(c2ccccc2)c2ccc3sc4c5ccccc5c(-c5cccc6c5sc5ccccc56)cc4c3c2)cc1 ; c1ccc(N(c2ccc3ccccc3c2)c2ccc3sc4c5ccccc5c(-c5cccc6c5sc5ccccc56)cc4c3c2)cc1 ; c1ccc(N(c2ccc3sc4c5ccccc5c(-c5cccc6c5sc5ccccc56)cc4c3c2)c2cccc3ccccc23)cc1 ; c1ccc2cc(N(c3ccc4ccccc4c3)c3ccc4sc5c6ccccc6c(-c6cccc7c6sc6ccccc67)cc5c4c3)ccc2c1]

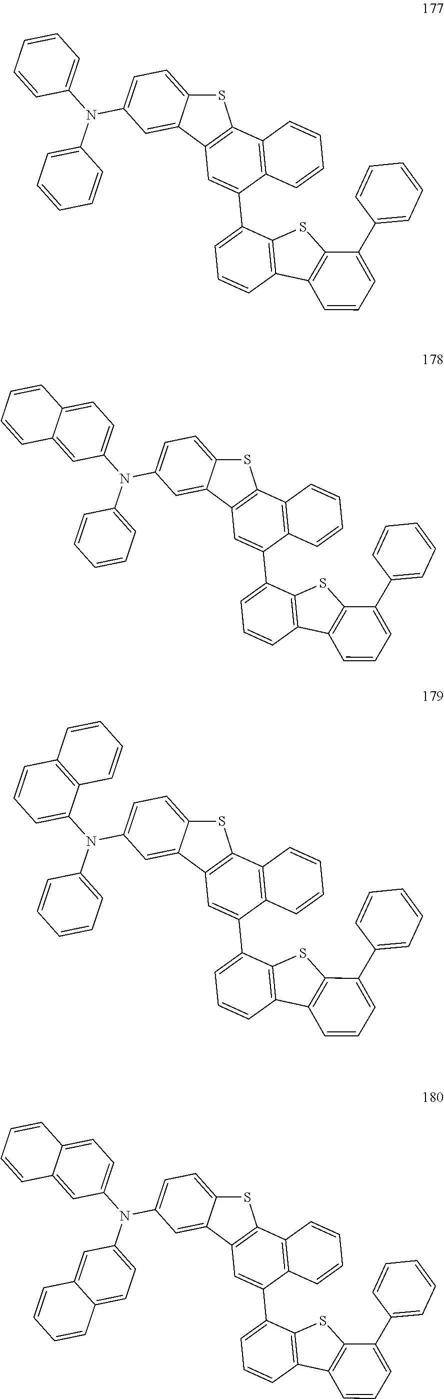 [SMILES: c1ccc(-c2cccc3c2sc2c(-c4cc5c6cc(N(c7ccccc7)c7ccccc7)ccc6sc5c5ccccc45)cccc23)cc1 ; c1ccc(-c2cccc3c2sc2c(-c4cc5c6cc(N(c7ccccc7)c7ccc8ccccc8c7)ccc6sc5c5ccccc45)cccc23)cc1 ; c1ccc(-c2cccc3c2sc2c(-c4cc5c6cc(N(c7ccccc7)c7cccc8ccccc78)ccc6sc5c5ccccc45)cccc23)cc1 ; c1ccc(-c2cccc3c2sc2c(-c4cc5c6cc(N(c7ccc8ccccc8c7)c7ccc8ccccc8c7)ccc6sc5c5ccccc45)cccc23)cc1]

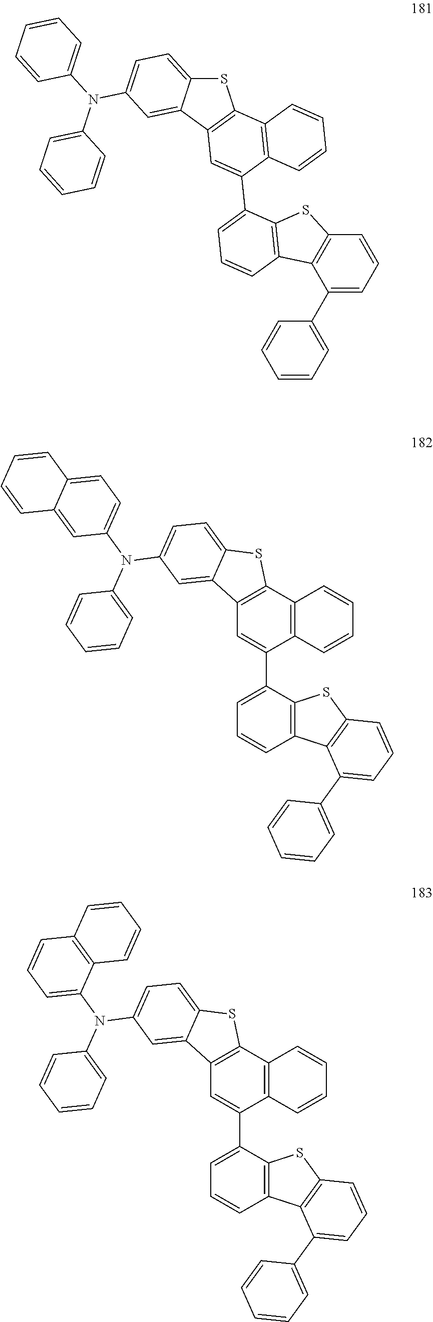 [SMILES: c1ccc(-c2cccc3sc4c(-c5cc6c7cc(N(c8ccccc8)c8ccccc8)ccc7sc6c6ccccc56)cccc4c23)cc1 ; c1ccc(-c2cccc3sc4c(-c5cc6c7cc(N(c8ccccc8)c8ccc9ccccc9c8)ccc7sc6c6ccccc56)cccc4c23)cc1 ; c1ccc(-c2cccc3sc4c(-c5cc6c7cc(N(c8ccccc8)c8cccc9ccccc89)ccc7sc6c6ccccc56)cccc4c23)cc1]

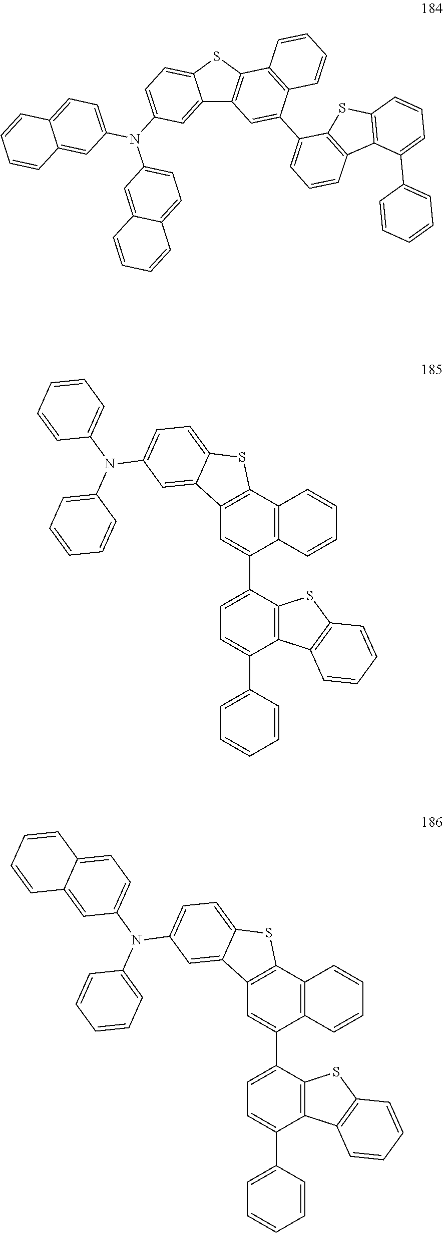 [SMILES: c1ccc(-c2cccc3sc4c(-c5cc6c7cc(N(c8ccc9ccccc9c8)c8ccc9ccccc9c8)ccc7sc6c6ccccc56)cccc4c23)cc1 ; c1ccc(-c2ccc(-c3cc4c5cc(N(c6ccccc6)c6ccccc6)ccc5sc4c4ccccc34)c3sc4ccccc4c23)cc1 ; c1ccc(-c2ccc(-c3cc4c5cc(N(c6ccccc6)c6ccc7ccccc7c6)ccc5sc4c4ccccc34)c3sc4ccccc4c23)cc1]

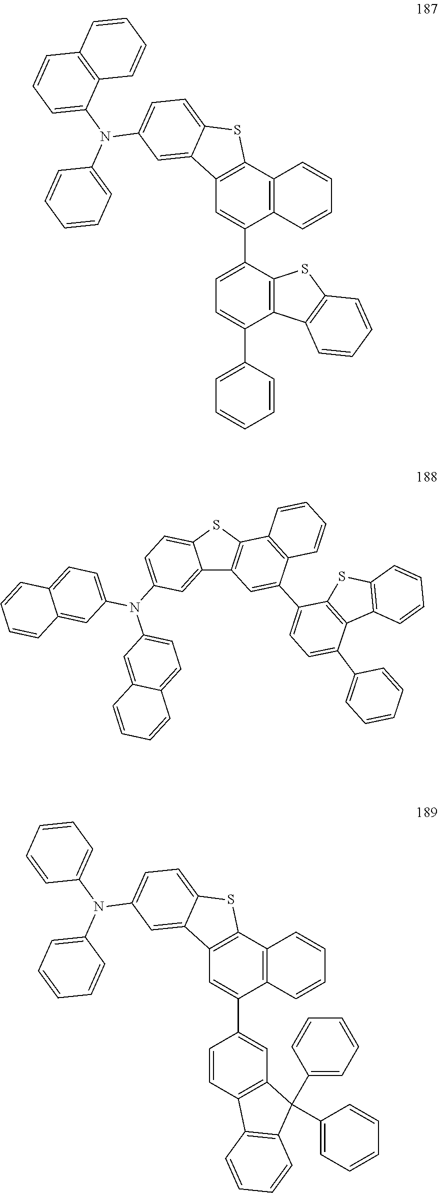 [SMILES: c1ccc(-c2ccc(-c3cc4c5cc(N(c6ccccc6)c6cccc7ccccc67)ccc5sc4c4ccccc34)c3sc4ccccc4c23)cc1 ; c1ccc(-c2ccc(-c3cc4c5cc(N(c6ccc7ccccc7c6)c6ccc7ccccc7c6)ccc5sc4c4ccccc34)c3sc4ccccc4c23)cc1 ; c1ccc(N(c2ccccc2)c2ccc3sc4c5ccccc5c(-c5ccc6c(c5)C(c5ccccc5)(c5ccccc5)c5ccccc5-6)cc4c3c2)cc1]

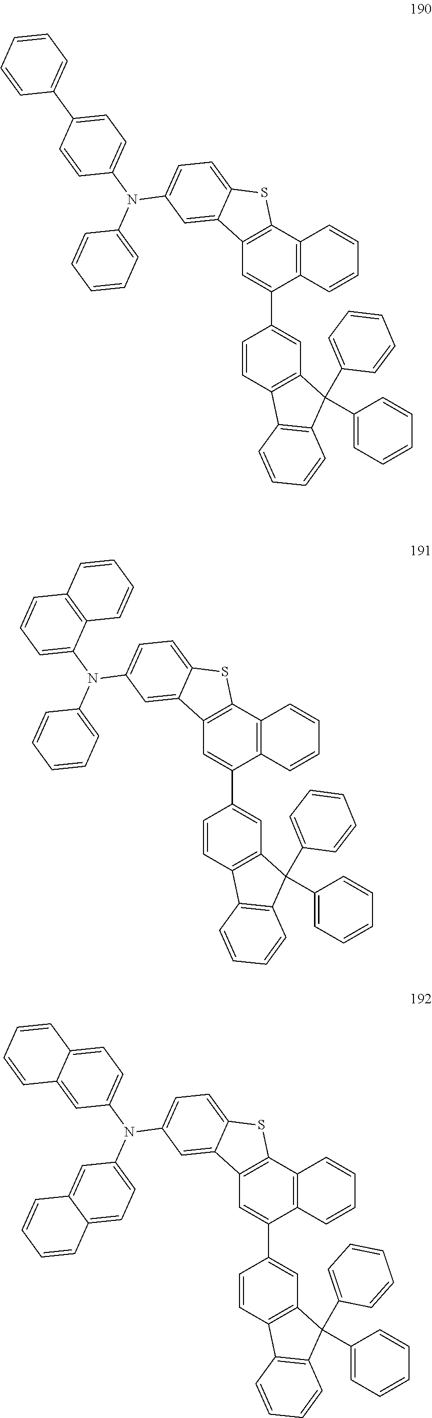 [SMILES: c1ccc(-c2ccc(N(c3ccccc3)c3ccc4sc5c6ccccc6c(-c6ccc7c(c6)C(c6ccccc6)(c6ccccc6)c6ccccc6-7)cc5c4c3)cc2)cc1 ; c1ccc(N(c2ccc3sc4c5ccccc5c(-c5ccc6c(c5)C(c5ccccc5)(c5ccccc5)c5ccccc5-6)cc4c3c2)c2cccc3ccccc23)cc1 ; c1ccc(C2(c3ccccc3)c3ccccc3-c3ccc(-c4cc5c6cc(N(c7ccc8ccccc8c7)c7ccc8ccccc8c7)ccc6sc5c5ccccc45)cc32)cc1]

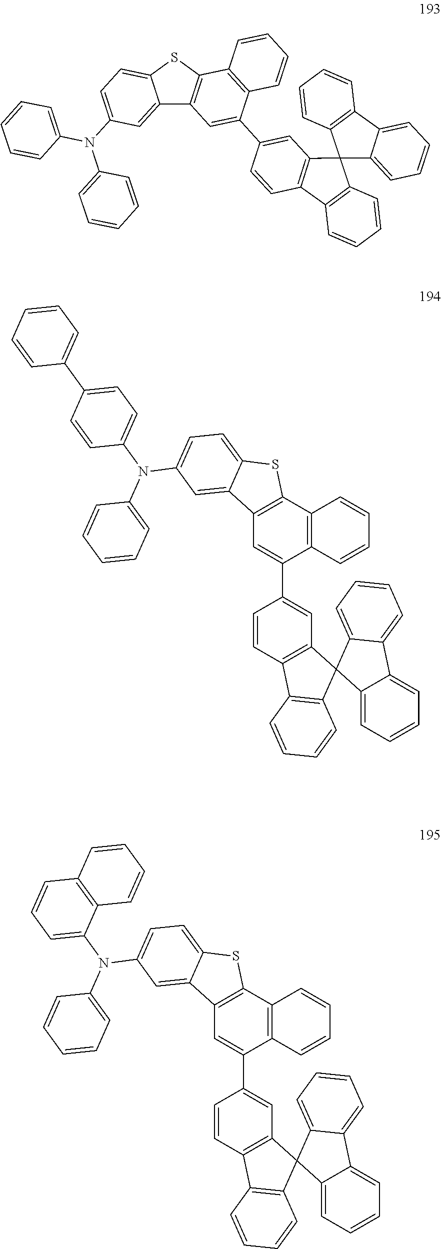 [SMILES: c1ccc(N(c2ccccc2)c2ccc3sc4c5ccccc5c(-c5ccc6c(c5)C5(c7ccccc7-c7ccccc75)c5ccccc5-6)cc4c3c2)cc1 ; c1ccc(-c2ccc(N(c3ccccc3)c3ccc4sc5c6ccccc6c(-c6ccc7c(c6)C6(c8ccccc8-c8ccccc86)c6ccccc6-7)cc5c4c3)cc2)cc1 ; c1ccc(N(c2ccc3sc4c5ccccc5c(-c5ccc6c(c5)C5(c7ccccc7-c7ccccc75)c5ccccc5-6)cc4c3c2)c2cccc3ccccc23)cc1]

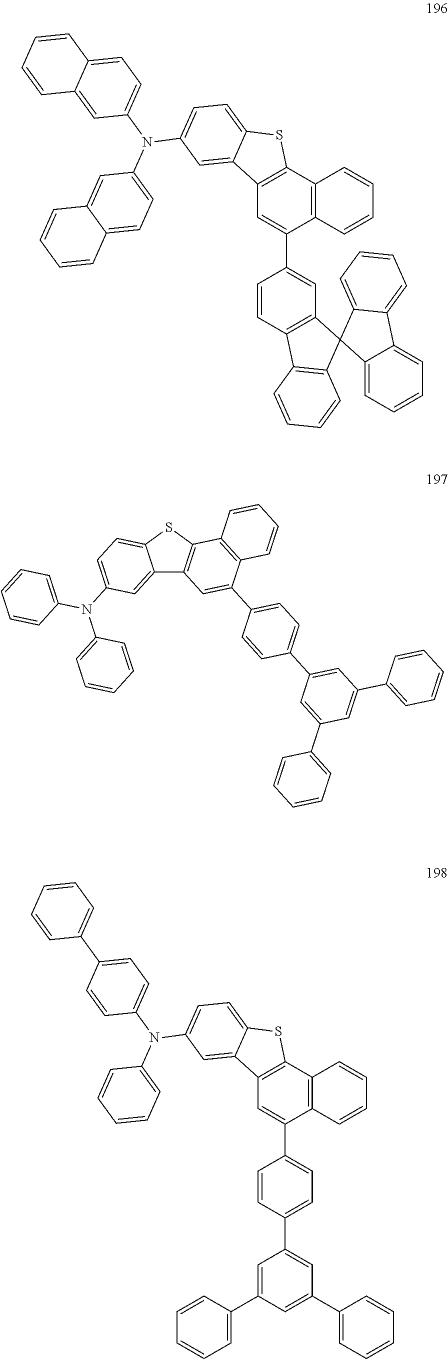

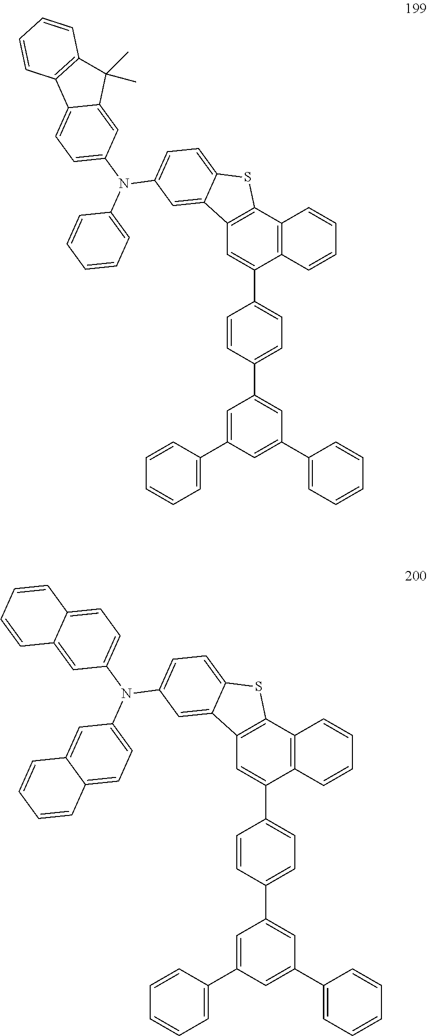 [SMILES: CC1(C)c2ccccc2-c2ccc(N(c3ccccc3)c3ccc4sc5c6ccccc6c(-c6ccc(-c7cc(-c8ccccc8)cc(-c8ccccc8)c7)cc6)cc5c4c3)cc21 ; c1ccc(-c2cc(-c3ccccc3)cc(-c3ccc(-c4cc5c6cc(N(c7ccc8ccccc8c7)c7ccc8ccccc8c7)ccc6sc5c5ccccc45)cc3)c2)cc1]

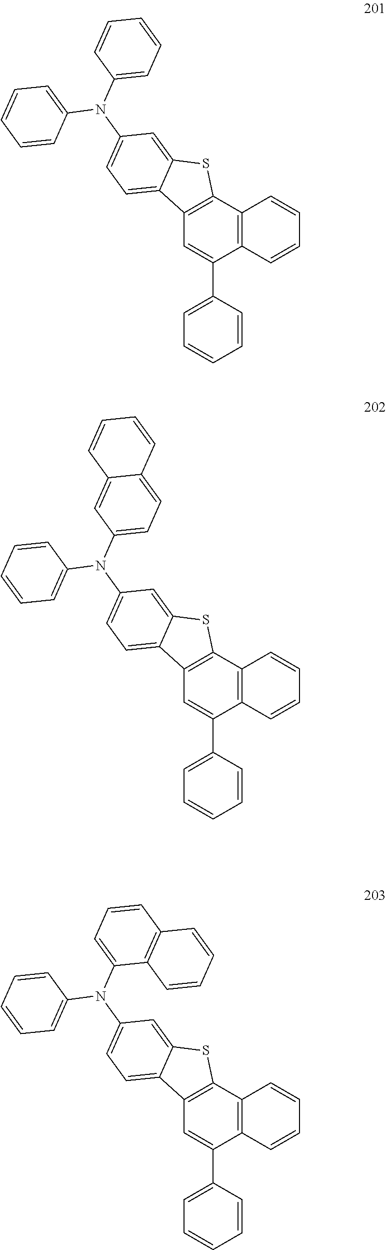 [SMILES: c1ccc(-c2cc3c4ccc(N(c5ccccc5)c5ccccc5)cc4sc3c3ccccc23)cc1 ; c1ccc(-c2cc3c4ccc(N(c5ccccc5)c5ccc6ccccc6c5)cc4sc3c3ccccc23)cc1 ; c1ccc(-c2cc3c4ccc(N(c5ccccc5)c5cccc6ccccc56)cc4sc3c3ccccc23)cc1]

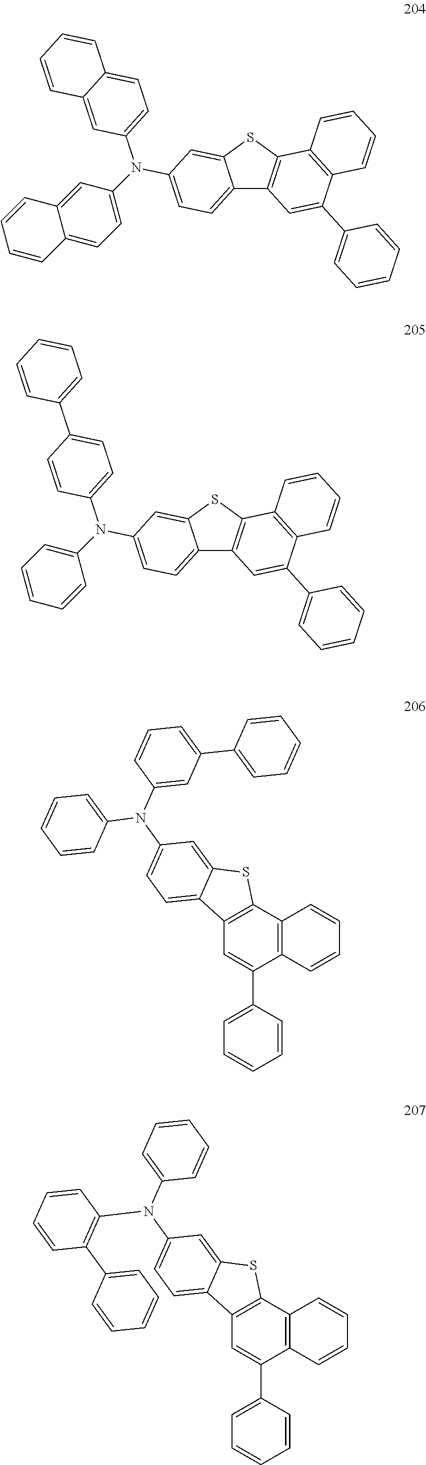 [SMILES: c1ccc(-c2cc3c4ccc(N(c5ccc6ccccc6c5)c5ccc6ccccc6c5)cc4sc3c3ccccc23)cc1 ; c1ccc(-c2ccc(N(c3ccccc3)c3ccc4c(c3)sc3c5ccccc5c(-c5ccccc5)cc43)cc2)cc1 ; c1ccc(-c2cccc(N(c3ccccc3)c3ccc4c(c3)sc3c5ccccc5c(-c5ccccc5)cc43)c2)cc1 ; c1ccc(-c2ccccc2N(c2ccccc2)c2ccc3c(c2)sc2c4ccccc4c(-c4ccccc4)cc32)cc1]

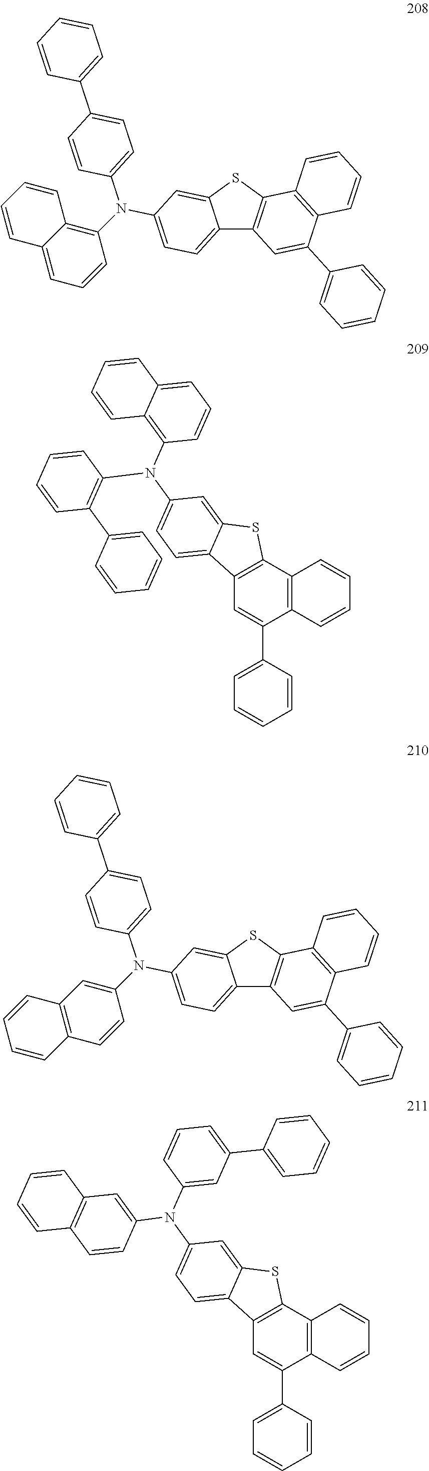 [SMILES: c1ccc(-c2ccc(N(c3ccc4c(c3)sc3c5ccccc5c(-c5ccccc5)cc43)c3cccc4ccccc34)cc2)cc1 ; c1ccc(-c2ccccc2N(c2ccc3c(c2)sc2c4ccccc4c(-c4ccccc4)cc32)c2cccc3ccccc23)cc1 ; c1ccc(-c2ccc(N(c3ccc4ccccc4c3)c3ccc4c(c3)sc3c5ccccc5c(-c5ccccc5)cc43)cc2)cc1 ; c1ccc(-c2cccc(N(c3ccc4ccccc4c3)c3ccc4c(c3)sc3c5ccccc5c(-c5ccccc5)cc43)c2)cc1]

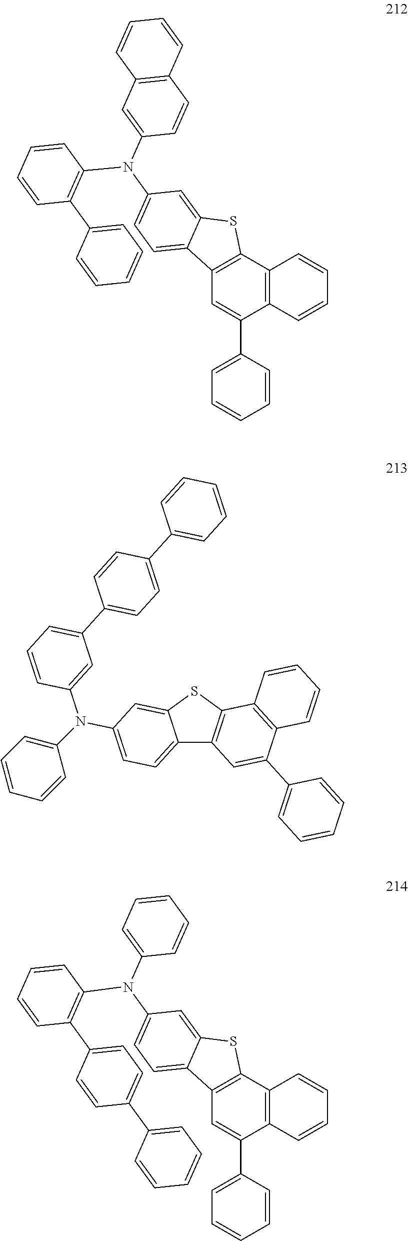 [SMILES: c1ccc(-c2ccccc2N(c2ccc3ccccc3c2)c2ccc3c(c2)sc2c4ccccc4c(-c4ccccc4)cc32)cc1 ; c1ccc(-c2ccc(-c3cccc(N(c4ccccc4)c4ccc5c(c4)sc4c6ccccc6c(-c6ccccc6)cc54)c3)cc2)cc1 ; c1ccc(-c2ccc(-c3ccccc3N(c3ccccc3)c3ccc4c(c3)sc3c5ccccc5c(-c5ccccc5)cc43)cc2)cc1]

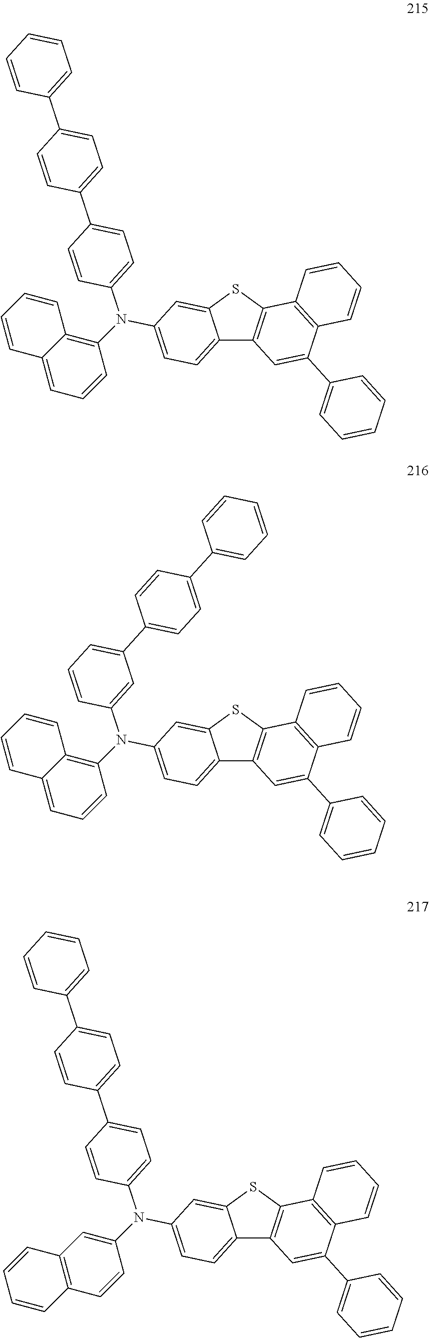 [SMILES: c1ccc(-c2ccc(-c3ccc(N(c4ccc5c(c4)sc4c6ccccc6c(-c6ccccc6)cc54)c4cccc5ccccc45)cc3)cc2)cc1 ; c1ccc(-c2ccc(-c3cccc(N(c4ccc5c(c4)sc4c6ccccc6c(-c6ccccc6)cc54)c4cccc5ccccc45)c3)cc2)cc1 ; c1ccc(-c2ccc(-c3ccc(N(c4ccc5ccccc5c4)c4ccc5c(c4)sc4c6ccccc6c(-c6ccccc6)cc54)cc3)cc2)cc1]

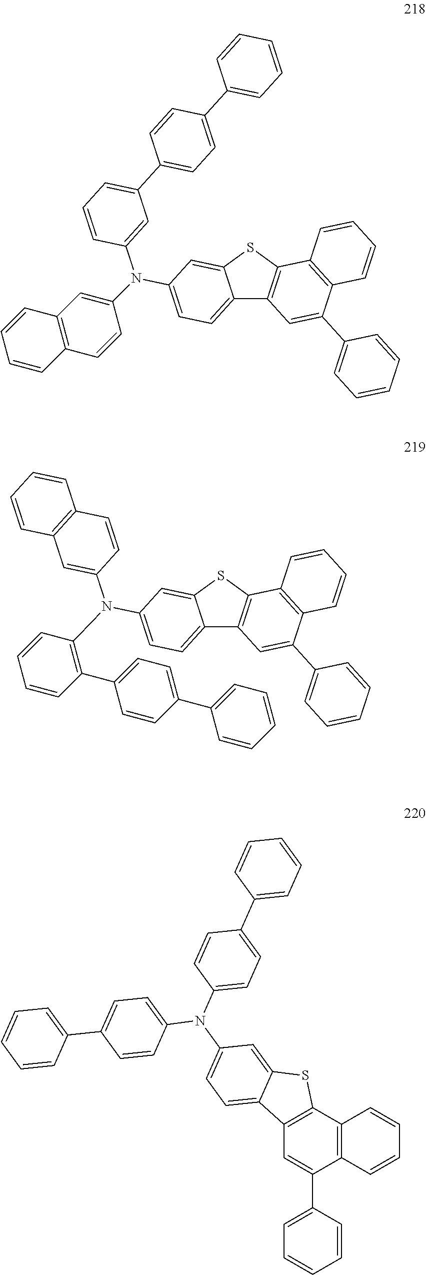 [SMILES: c1ccc(-c2ccc(-c3cccc(N(c4ccc5ccccc5c4)c4ccc5c(c4)sc4c6ccccc6c(-c6ccccc6)cc54)c3)cc2)cc1 ; c1ccc(-c2ccc(-c3ccccc3N(c3ccc4ccccc4c3)c3ccc4c(c3)sc3c5ccccc5c(-c5ccccc5)cc43)cc2)cc1 ; c1ccc(-c2ccc(N(c3ccc(-c4ccccc4)cc3)c3ccc4c(c3)sc3c5ccccc5c(-c5ccccc5)cc43)cc2)cc1]

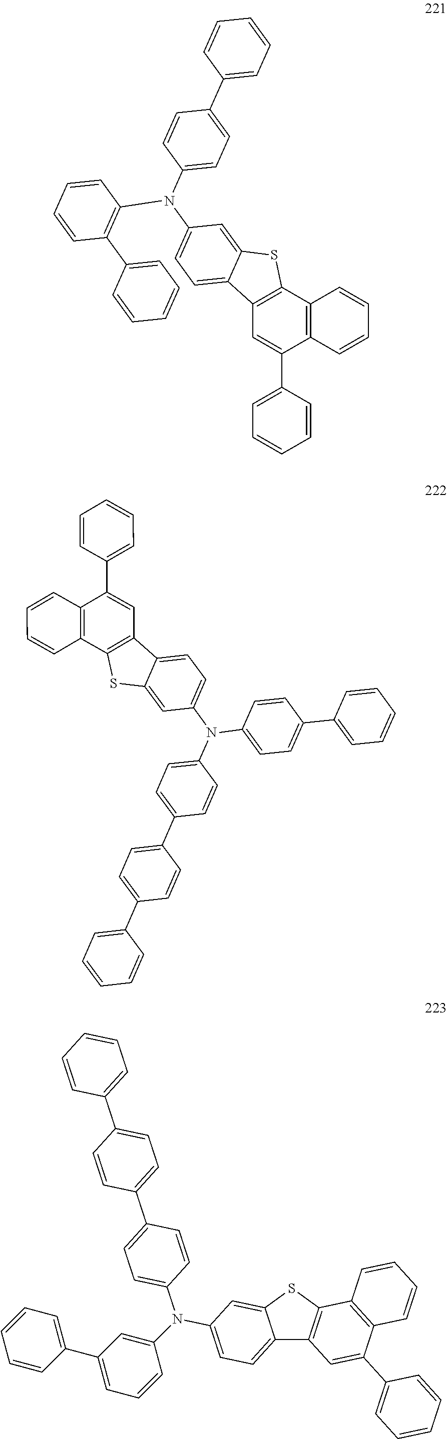 [SMILES: c1ccc(-c2ccc(N(c3ccc4c(c3)sc3c5ccccc5c(-c5ccccc5)cc43)c3ccccc3-c3ccccc3)cc2)cc1 ; c1ccc(-c2ccc(-c3ccc(N(c4ccc(-c5ccccc5)cc4)c4ccc5c(c4)sc4c6ccccc6c(-c6ccccc6)cc54)cc3)cc2)cc1 ; c1ccc(-c2ccc(-c3ccc(N(c4cccc(-c5ccccc5)c4)c4ccc5c(c4)sc4c6ccccc6c(-c6ccccc6)cc54)cc3)cc2)cc1]

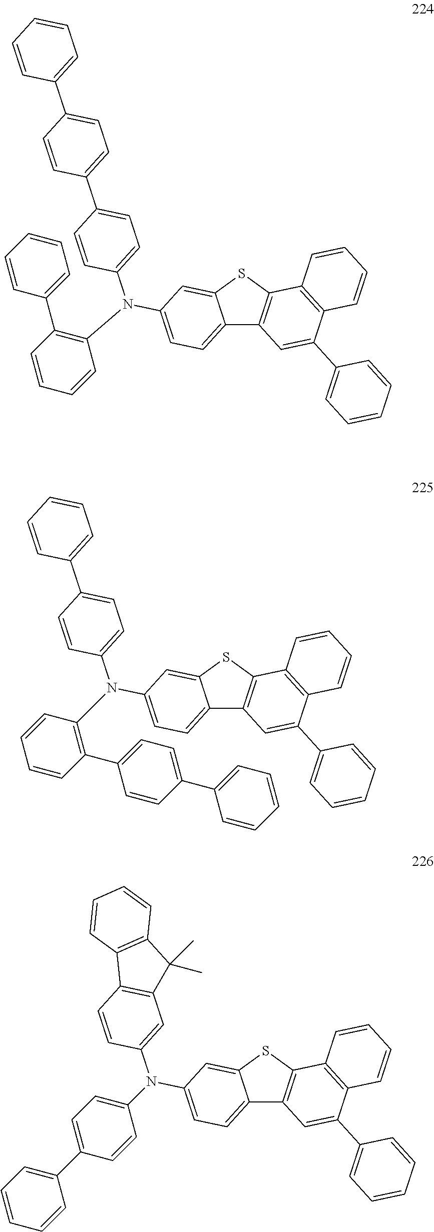 [SMILES: c1ccc(-c2ccc(-c3ccc(N(c4ccc5c(c4)sc4c6ccccc6c(-c6ccccc6)cc54)c4ccccc4-c4ccccc4)cc3)cc2)cc1 ; c1ccc(-c2ccc(-c3ccccc3N(c3ccc(-c4ccccc4)cc3)c3ccc4c(c3)sc3c5ccccc5c(-c5ccccc5)cc43)cc2)cc1 ; CC1(C)c2ccccc2-c2ccc(N(c3ccc(-c4ccccc4)cc3)c3ccc4c(c3)sc3c5ccccc5c(-c5ccccc5)cc43)cc21]

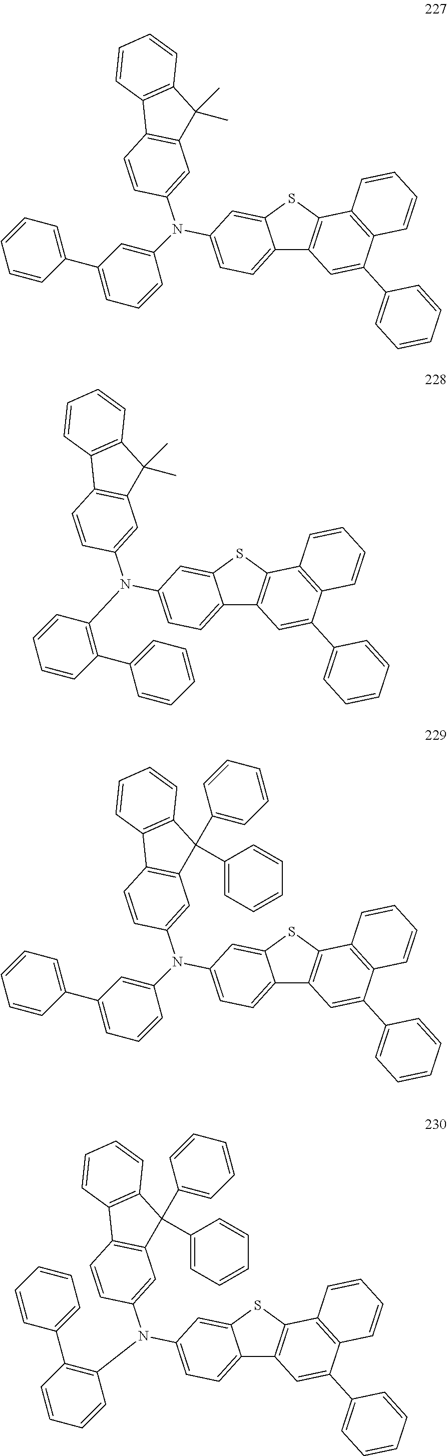 [SMILES: CC1(C)c2ccccc2-c2ccc(N(c3cccc(-c4ccccc4)c3)c3ccc4c(c3)sc3c5ccccc5c(-c5ccccc5)cc43)cc21 ; CC1(C)c2ccccc2-c2ccc(N(c3ccc4c(c3)sc3c5ccccc5c(-c5ccccc5)cc43)c3ccccc3-c3ccccc3)cc21 ; c1ccc(-c2cccc(N(c3ccc4c(c3)C(c3ccccc3)(c3ccccc3)c3ccccc3-4)c3ccc4c(c3)sc3c5ccccc5c(-c5ccccc5)cc43)c2)cc1 ; c1ccc(-c2ccccc2N(c2ccc3c(c2)C(c2ccccc2)(c2ccccc2)c2ccccc2-3)c2ccc3c(c2)sc2c4ccccc4c(-c4ccccc4)cc32)cc1]

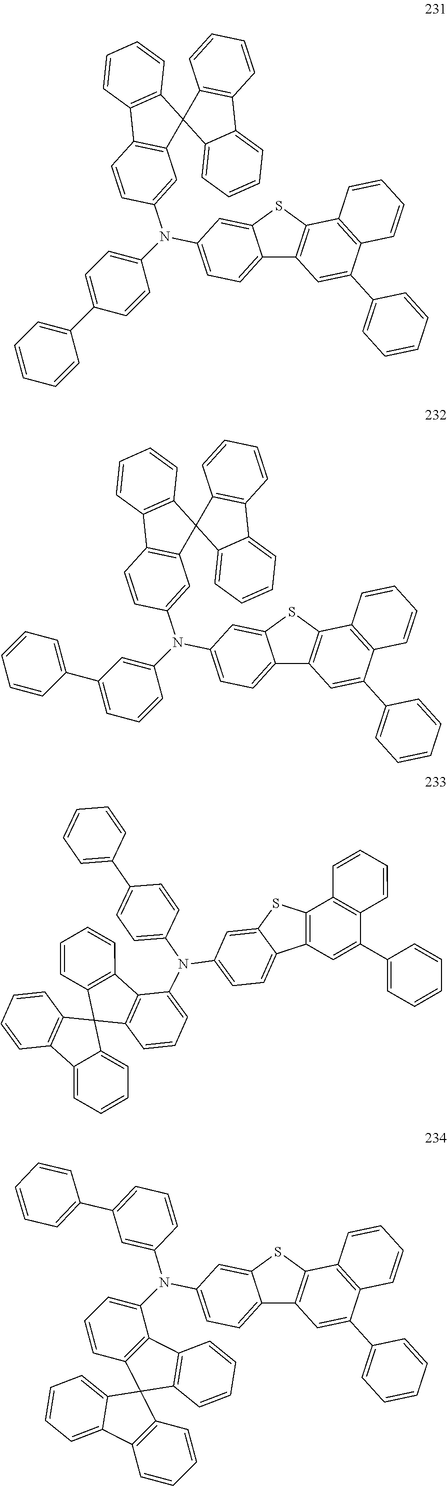 [SMILES: c1ccc(-c2ccc(N(c3ccc4c(c3)C3(c5ccccc5-c5ccccc53)c3ccccc3-4)c3ccc4c(c3)sc3c5ccccc5c(-c5ccccc5)cc43)cc2)cc1 ; c1ccc(-c2cccc(N(c3ccc4c(c3)C3(c5ccccc5-c5ccccc53)c3ccccc3-4)c3ccc4c(c3)sc3c5ccccc5c(-c5ccccc5)cc43)c2)cc1 ; c1ccc(-c2ccc(N(c3ccc4c(c3)sc3c5ccccc5c(-c5ccccc5)cc43)c3cccc4c3-c3ccccc3C43c4ccccc4-c4ccccc43)cc2)cc1 ; c1ccc(-c2cccc(N(c3ccc4c(c3)sc3c5ccccc5c(-c5ccccc5)cc43)c3cccc4c3-c3ccccc3C43c4ccccc4-c4ccccc43)c2)cc1]

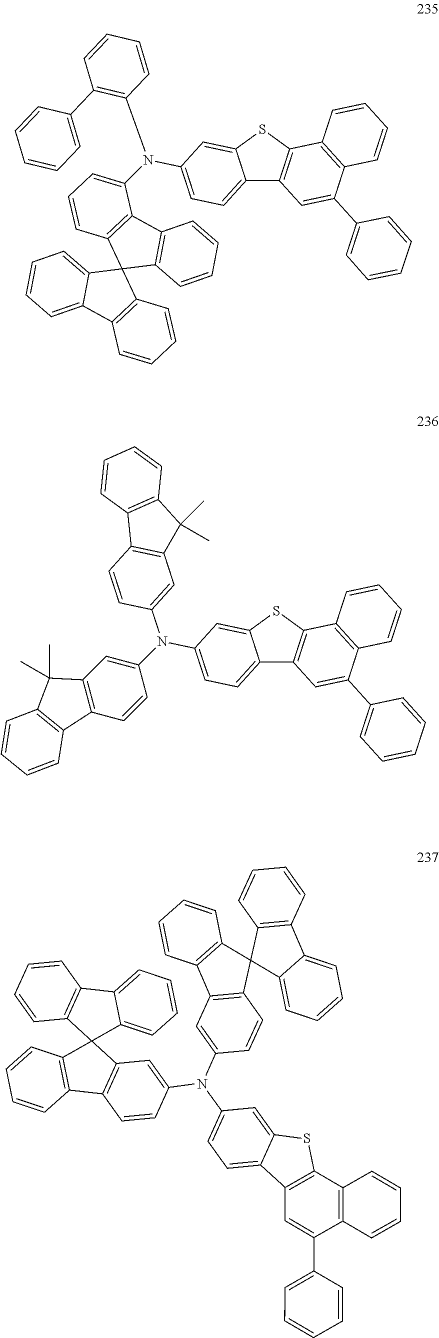 [SMILES: c1ccc(-c2ccccc2N(c2ccc3c(c2)sc2c4ccccc4c(-c4ccccc4)cc32)c2cccc3c2-c2ccccc2C32c3ccccc3-c3ccccc32)cc1 ; CC1(C)c2ccccc2-c2ccc(N(c3ccc4c(c3)C(C)(C)c3ccccc3-4)c3ccc4c(c3)sc3c5ccccc5c(-c5ccccc5)cc43)cc21 ; c1ccc(-c2cc3c4ccc(N(c5ccc6c(c5)-c5ccccc5C65c6ccccc6-c6ccccc65)c5ccc6c(c5)C5(c7ccccc7-c7ccccc75)c5ccccc5-6)cc4sc3c3ccccc23)cc1]

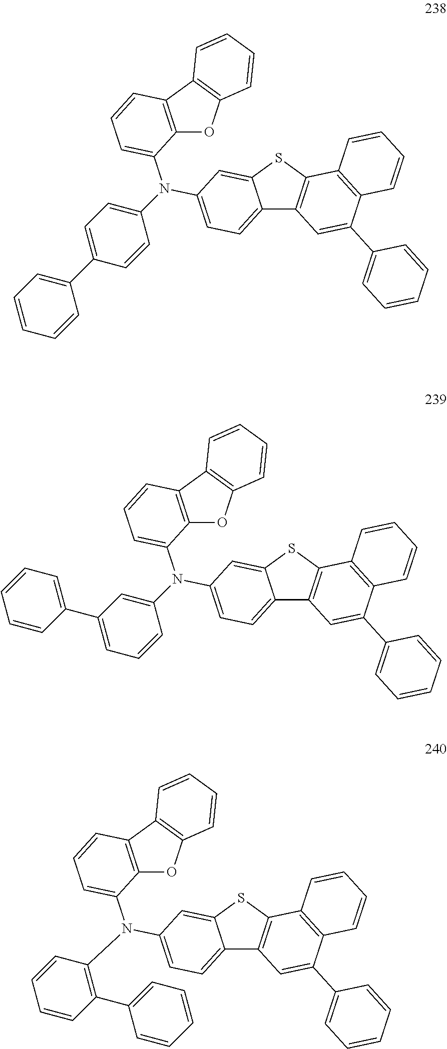 [SMILES: c1ccc(-c2ccc(N(c3ccc4c(c3)sc3c5ccccc5c(-c5ccccc5)cc43)c3cccc4c3oc3ccccc34)cc2)cc1 ; c1ccc(-c2cccc(N(c3ccc4c(c3)sc3c5ccccc5c(-c5ccccc5)cc43)c3cccc4c3oc3ccccc34)c2)cc1 ; c1ccc(-c2ccccc2N(c2ccc3c(c2)sc2c4ccccc4c(-c4ccccc4)cc32)c2cccc3c2oc2ccccc23)cc1]

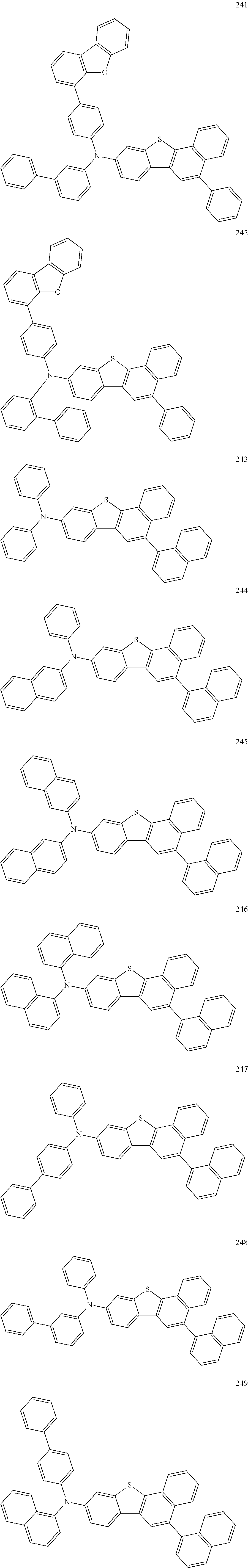

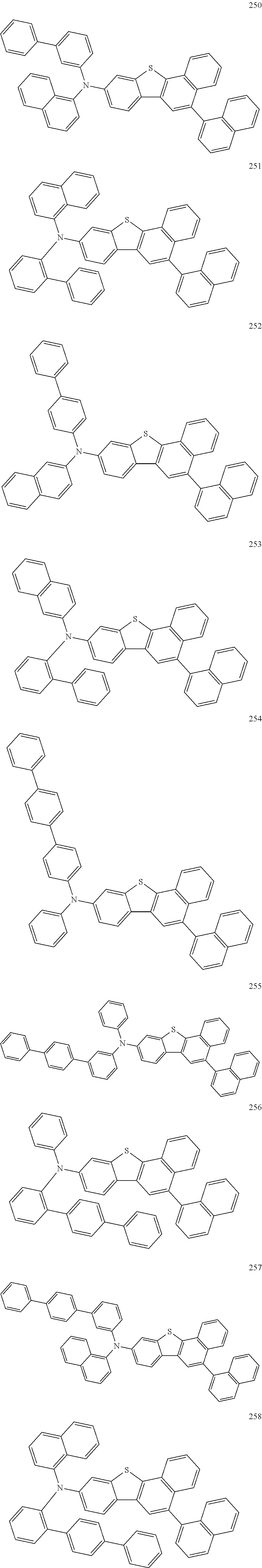

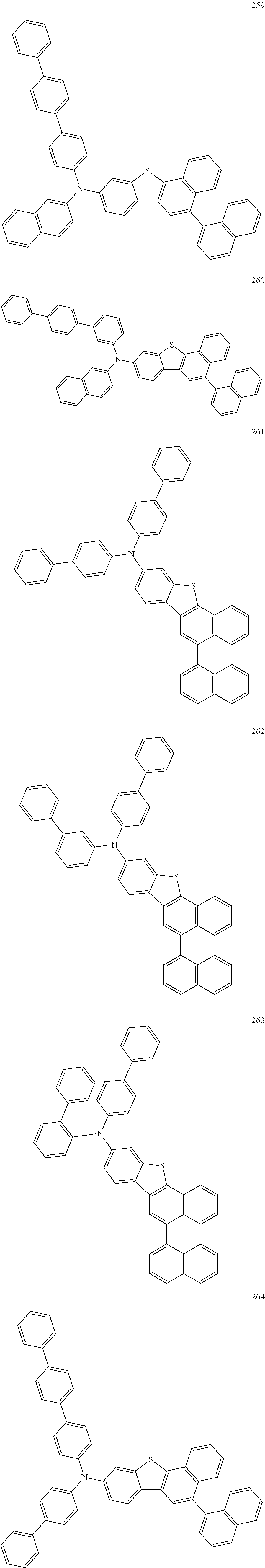

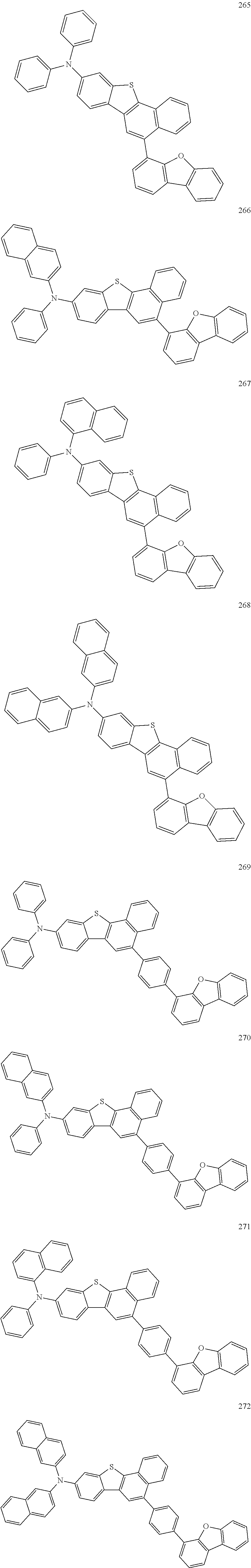

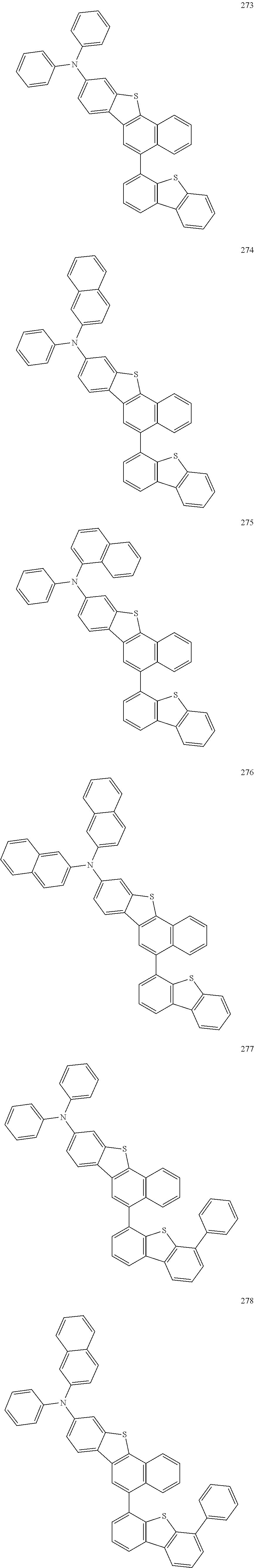 [SMILES: c1ccc(N(c2ccccc2)c2ccc3c(c2)sc2c4ccccc4c(-c4cccc5c4sc4ccccc45)cc32)cc1 ; c1ccc(N(c2ccc3ccccc3c2)c2ccc3c(c2)sc2c4ccccc4c(-c4cccc5c4sc4ccccc45)cc32)cc1 ; c1ccc(N(c2ccc3c(c2)sc2c4ccccc4c(-c4cccc5c4sc4ccccc45)cc32)c2cccc3ccccc23)cc1 ; c1ccc2cc(N(c3ccc4ccccc4c3)c3ccc4c(c3)sc3c5ccccc5c(-c5cccc6c5sc5ccccc56)cc43)ccc2c1 ; c1ccc(-c2cccc3c2sc2c(-c4cc5c6ccc(N(c7ccccc7)c7ccccc7)cc6sc5c5ccccc45)cccc23)cc1 ; c1ccc(-c2cccc3c2sc2c(-c4cc5c6ccc(N(c7ccccc7)c7ccc8ccccc8c7)cc6sc5c5ccccc45)cccc23)cc1]

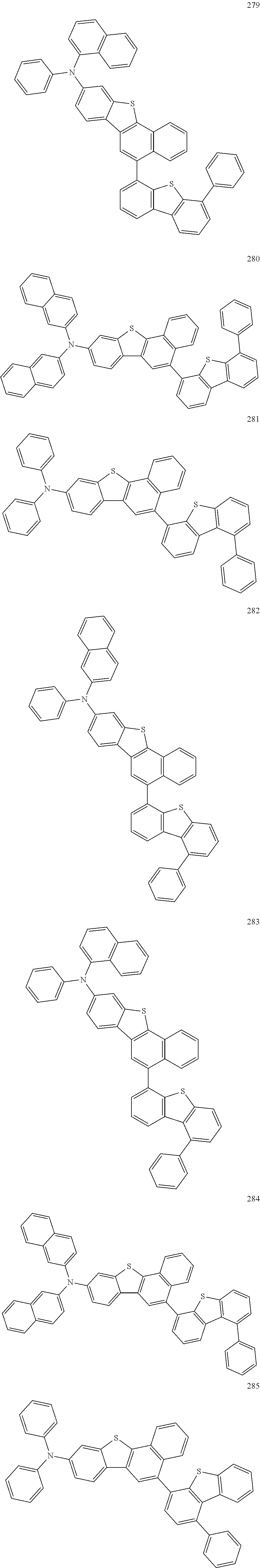

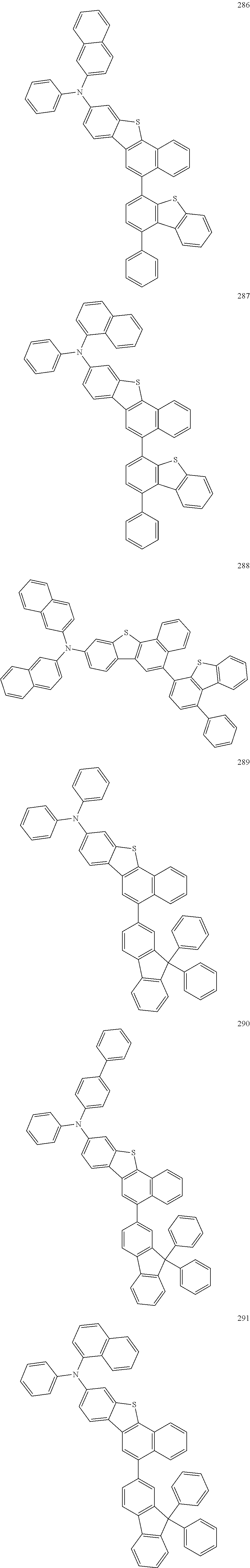

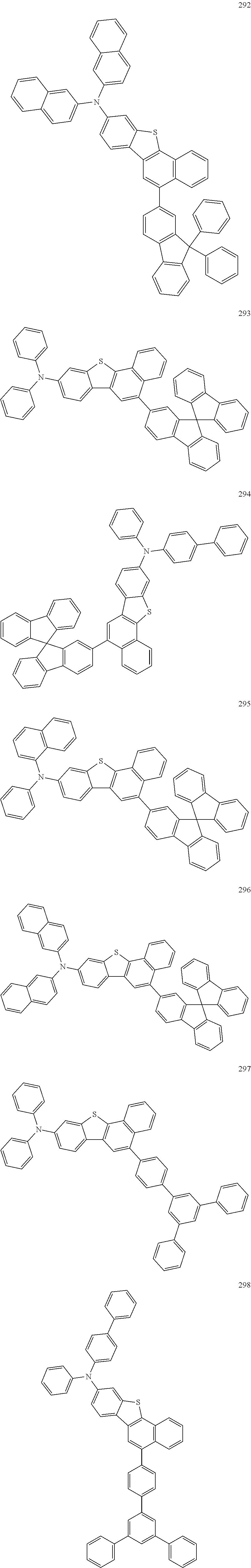

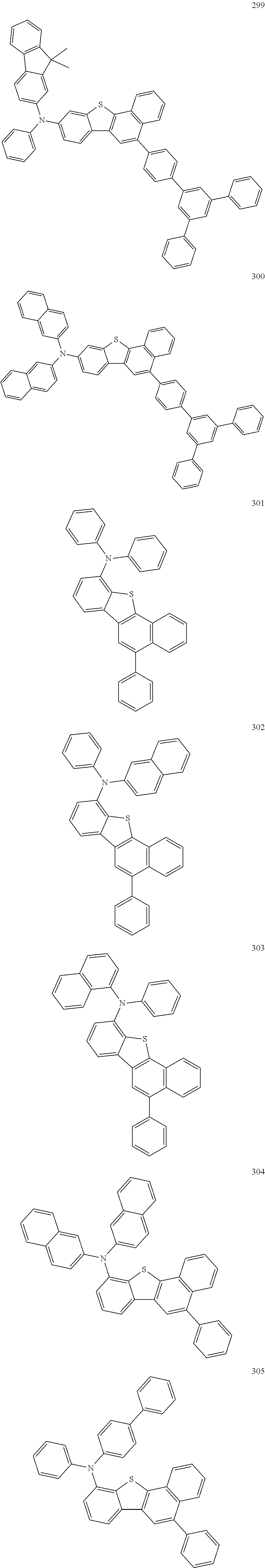

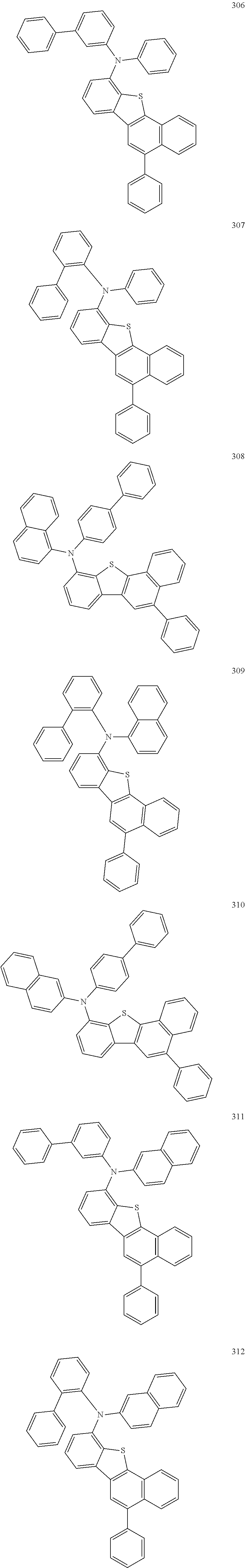

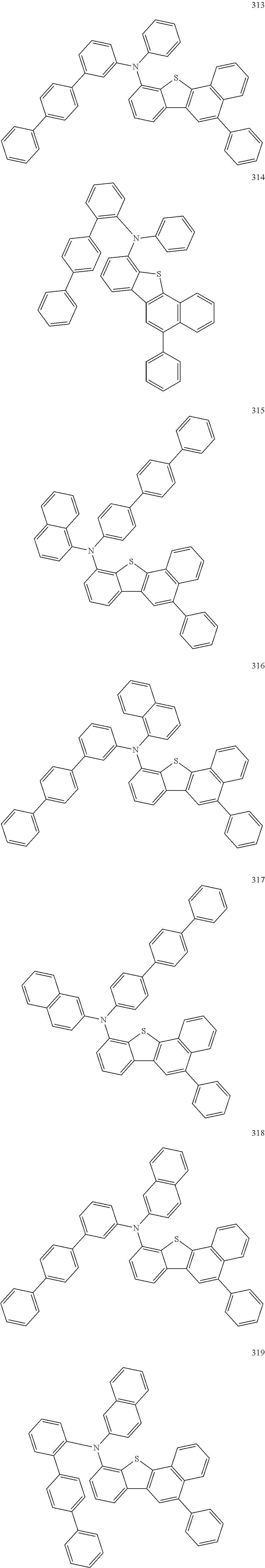

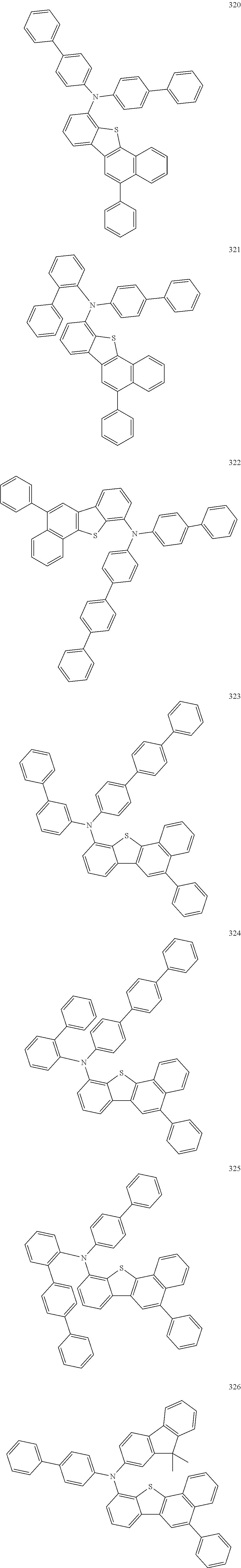

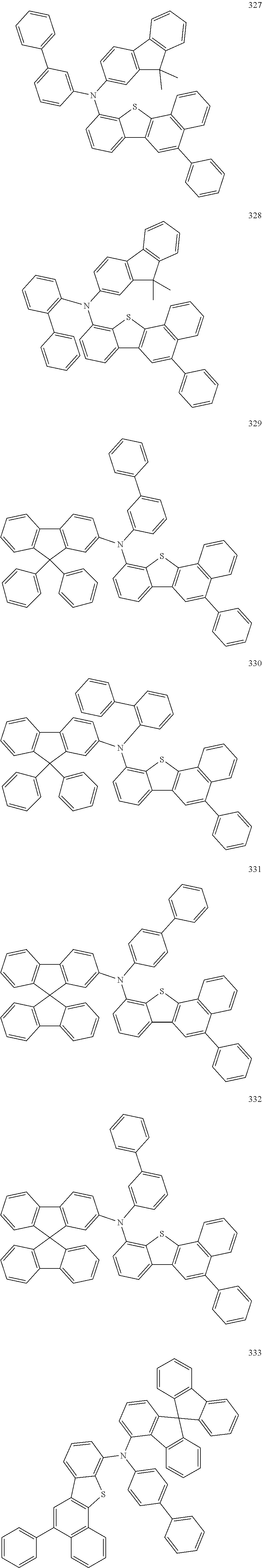

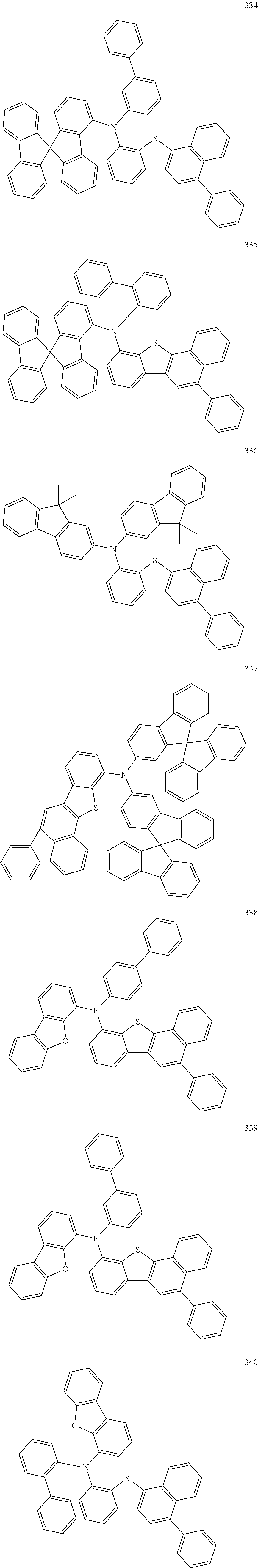

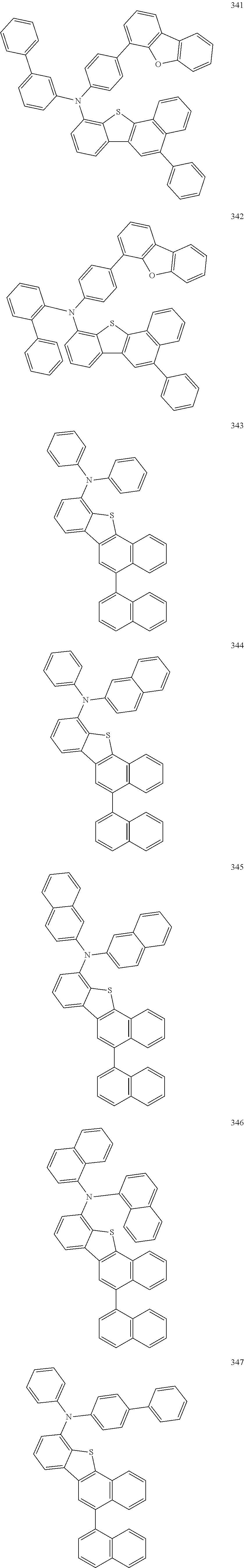

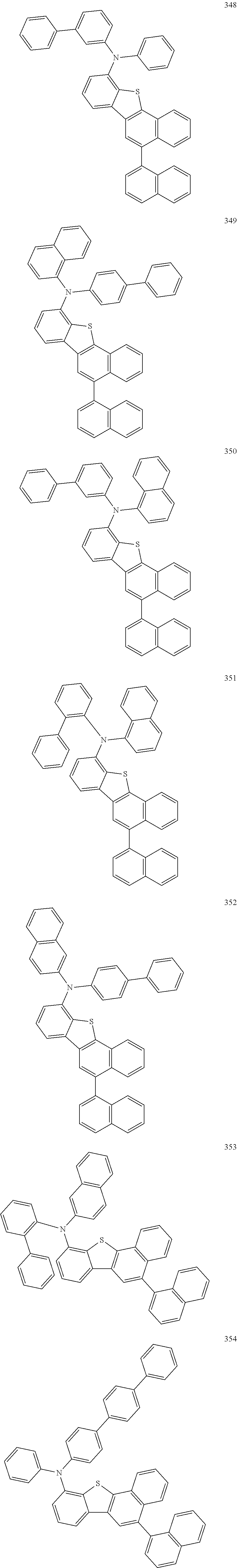

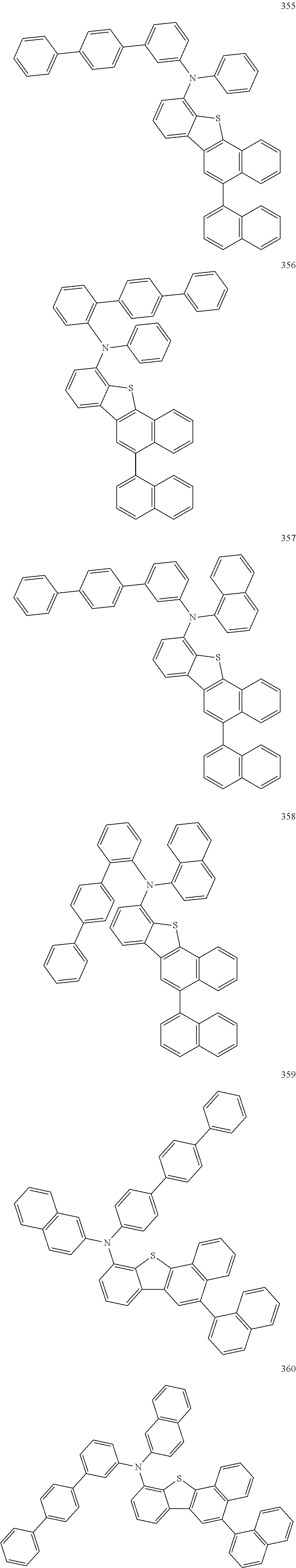

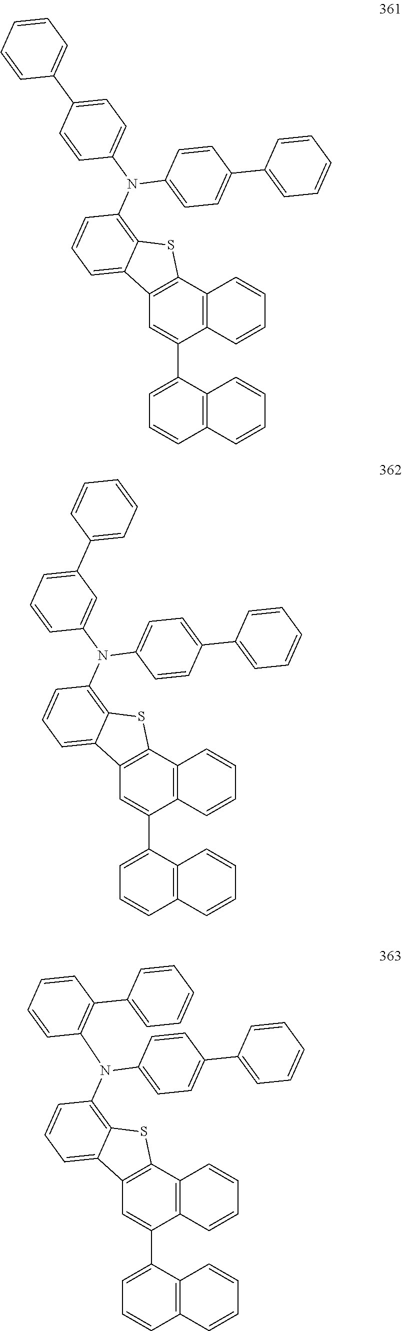 [SMILES: c1ccc(-c2ccc(N(c3ccc(-c4ccccc4)cc3)c3cccc4c3sc3c5ccccc5c(-c5cccc6ccccc56)cc43)cc2)cc1 ; c1ccc(-c2ccc(N(c3cccc(-c4ccccc4)c3)c3cccc4c3sc3c5ccccc5c(-c5cccc6ccccc56)cc43)cc2)cc1 ; c1ccc(-c2ccc(N(c3ccccc3-c3ccccc3)c3cccc4c3sc3c5ccccc5c(-c5cccc6ccccc56)cc43)cc2)cc1]

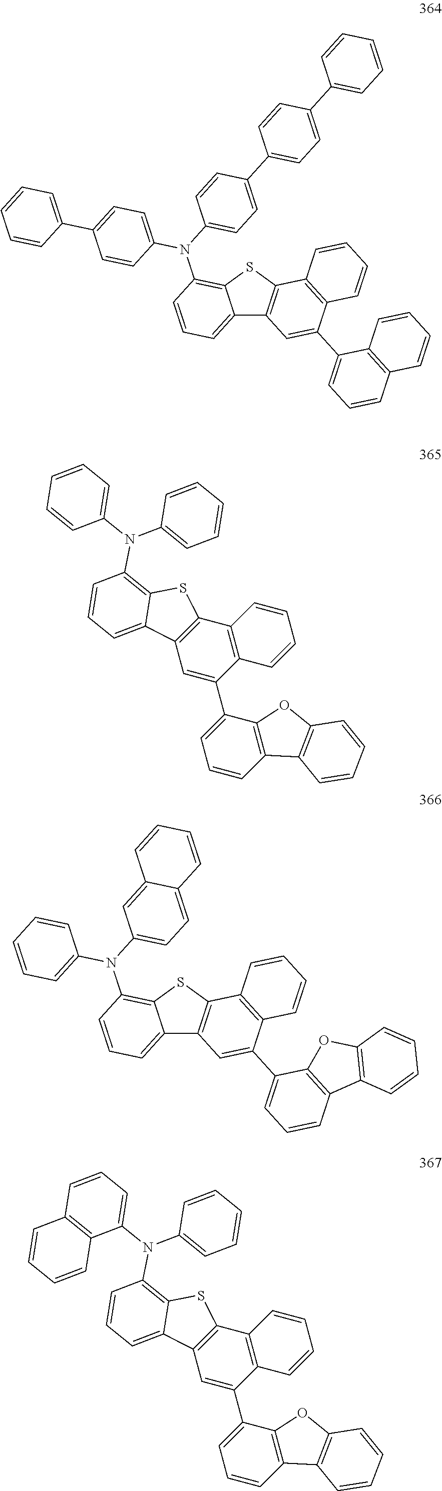 [SMILES: c1ccc(-c2ccc(-c3ccc(N(c4ccc(-c5ccccc5)cc4)c4cccc5c4sc4c6ccccc6c(-c6cccc7ccccc67)cc54)cc3)cc2)cc1 ; c1ccc(N(c2ccccc2)c2cccc3c2sc2c4ccccc4c(-c4cccc5c4oc4ccccc45)cc32)cc1 ; c1ccc(N(c2ccc3ccccc3c2)c2cccc3c2sc2c4ccccc4c(-c4cccc5c4oc4ccccc45)cc32)cc1 ; c1ccc(N(c2cccc3ccccc23)c2cccc3c2sc2c4ccccc4c(-c4cccc5c4oc4ccccc45)cc32)cc1]

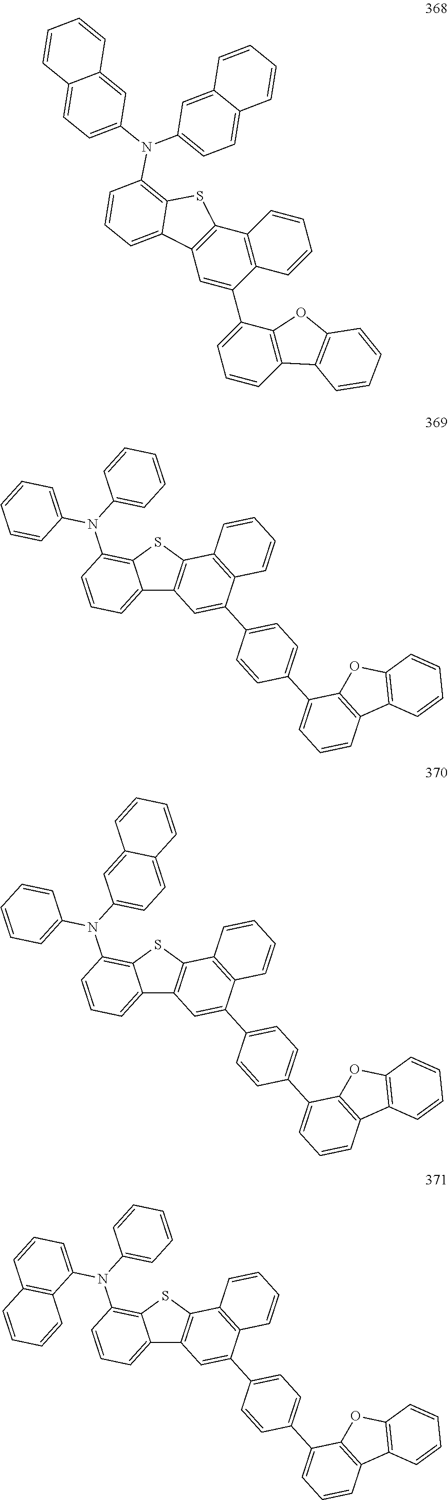 [SMILES: c1ccc2cc(N(c3ccc4ccccc4c3)c3cccc4c3sc3c5ccccc5c(-c5cccc6c5oc5ccccc56)cc43)ccc2c1 ; c1ccc(N(c2ccccc2)c2cccc3c2sc2c4ccccc4c(-c4ccc(-c5cccc6c5oc5ccccc56)cc4)cc32)cc1 ; c1ccc(N(c2ccc3ccccc3c2)c2cccc3c2sc2c4ccccc4c(-c4ccc(-c5cccc6c5oc5ccccc56)cc4)cc32)cc1 ; c1ccc(N(c2cccc3ccccc23)c2cccc3c2sc2c4ccccc4c(-c4ccc(-c5cccc6c5oc5ccccc56)cc4)cc32)cc1]

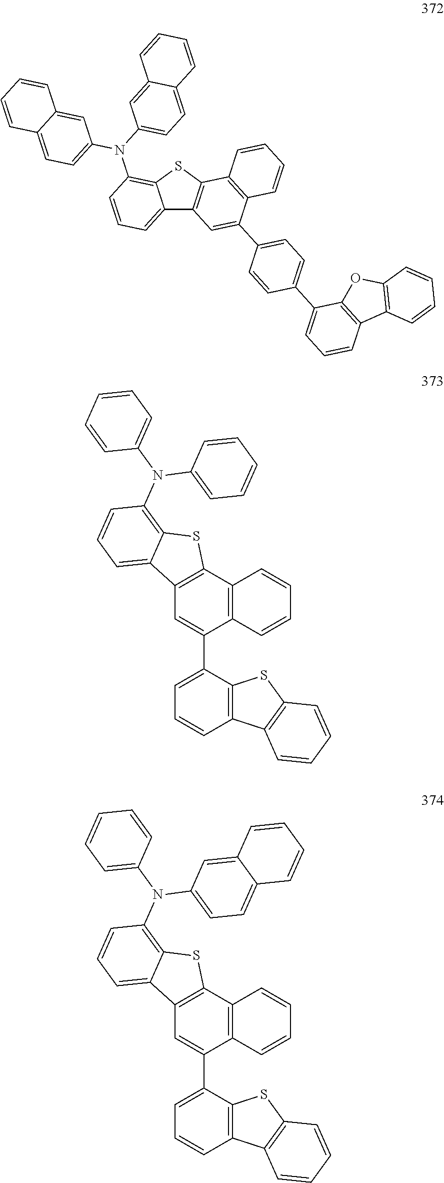 [SMILES: c1ccc2cc(N(c3ccc4ccccc4c3)c3cccc4c3sc3c5ccccc5c(-c5ccc(-c6cccc7c6oc6ccccc67)cc5)cc43)ccc2c1 ; c1ccc(N(c2ccccc2)c2cccc3c2sc2c4ccccc4c(-c4cccc5c4sc4ccccc45)cc32)cc1 ; c1ccc(N(c2ccc3ccccc3c2)c2cccc3c2sc2c4ccccc4c(-c4cccc5c4sc4ccccc45)cc32)cc1]

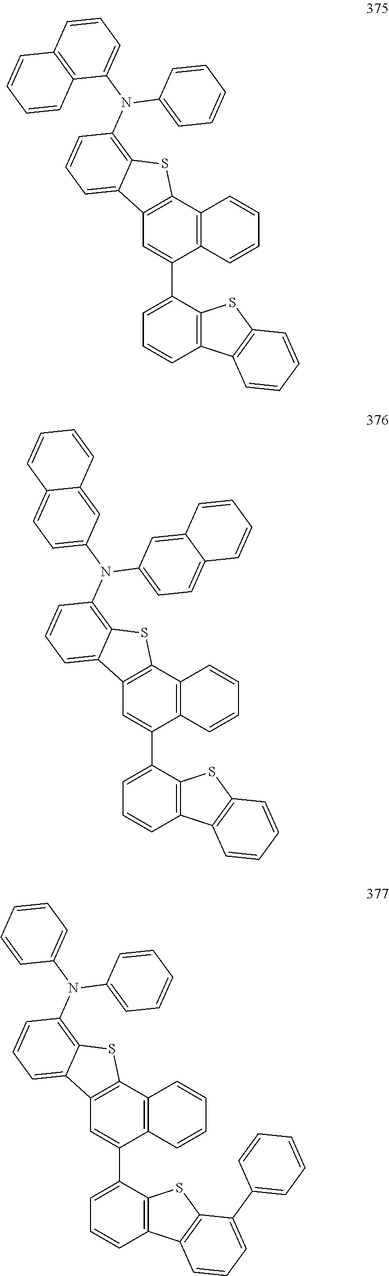 [SMILES: c1ccc(N(c2cccc3ccccc23)c2cccc3c2sc2c4ccccc4c(-c4cccc5c4sc4ccccc45)cc32)cc1 ; c1ccc2cc(N(c3ccc4ccccc4c3)c3cccc4c3sc3c5ccccc5c(-c5cccc6c5sc5ccccc56)cc43)ccc2c1 ; c1ccc(-c2cccc3c2sc2c(-c4cc5c6cccc(N(c7ccccc7)c7ccccc7)c6sc5c5ccccc45)cccc23)cc1]

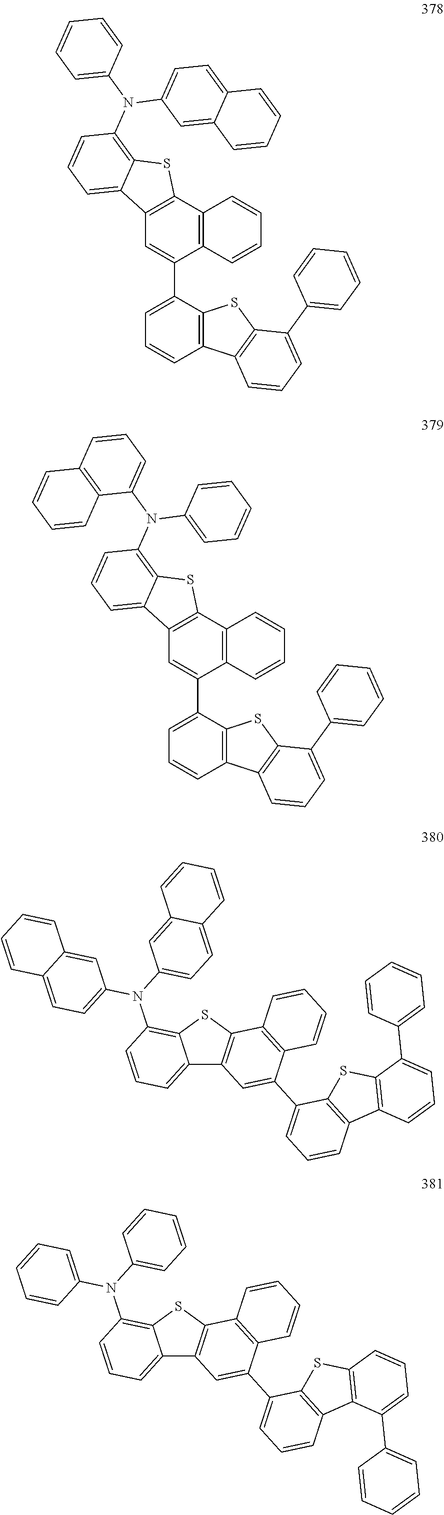 [SMILES: c1ccc(-c2cccc3c2sc2c(-c4cc5c6cccc(N(c7ccccc7)c7ccc8ccccc8c7)c6sc5c5ccccc45)cccc23)cc1 ; c1ccc(-c2cccc3c2sc2c(-c4cc5c6cccc(N(c7ccccc7)c7cccc8ccccc78)c6sc5c5ccccc45)cccc23)cc1 ; c1ccc(-c2cccc3c2sc2c(-c4cc5c6cccc(N(c7ccc8ccccc8c7)c7ccc8ccccc8c7)c6sc5c5ccccc45)cccc23)cc1 ; c1ccc(-c2cccc3sc4c(-c5cc6c7cccc(N(c8ccccc8)c8ccccc8)c7sc6c6ccccc56)cccc4c23)cc1]

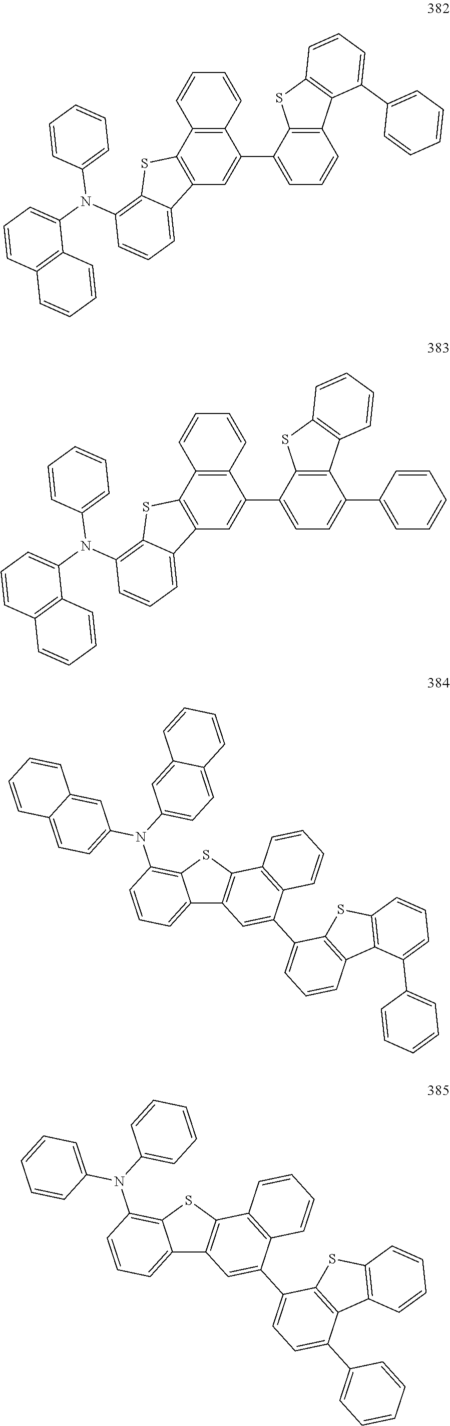 [SMILES: c1ccc(-c2cccc3sc4c(-c5cc6c7cccc(N(c8ccccc8)c8cccc9ccccc89)c7sc6c6ccccc56)cccc4c23)cc1 ; c1ccc(-c2ccc(-c3cc4c5cccc(N(c6ccccc6)c6cccc7ccccc67)c5sc4c4ccccc34)c3sc4ccccc4c23)cc1 ; c1ccc(-c2cccc3sc4c(-c5cc6c7cccc(N(c8ccc9ccccc9c8)c8ccc9ccccc9c8)c7sc6c6ccccc56)cccc4c23)cc1 ; c1ccc(-c2ccc(-c3cc4c5cccc(N(c6ccccc6)c6ccccc6)c5sc4c4ccccc34)c3sc4ccccc4c23)cc1]

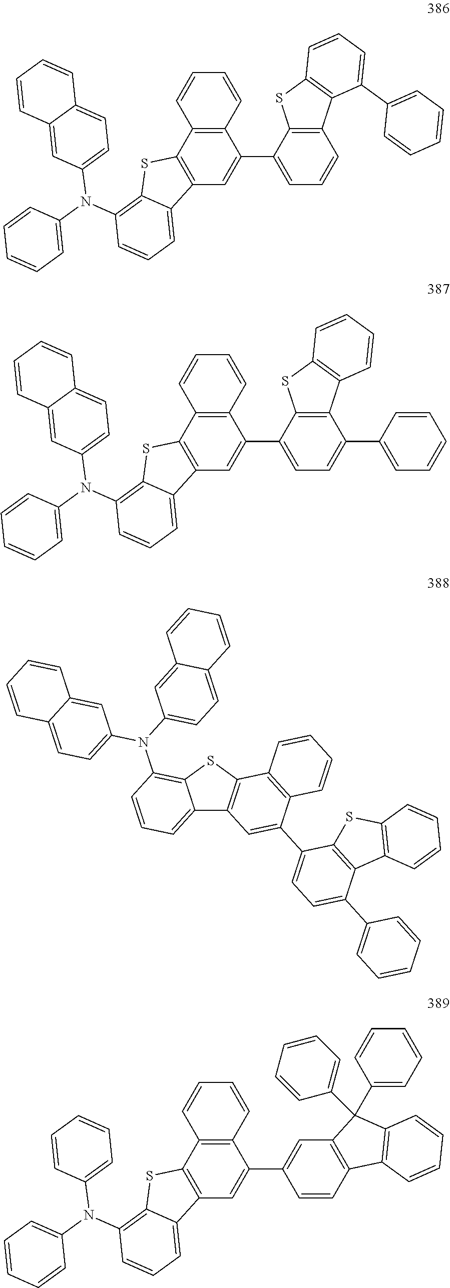 [SMILES: c1ccc(-c2cccc3sc4c(-c5cc6c7cccc(N(c8ccccc8)c8ccc9ccccc9c8)c7sc6c6ccccc56)cccc4c23)cc1 ; c1ccc(-c2ccc(-c3cc4c5cccc(N(c6ccccc6)c6ccc7ccccc7c6)c5sc4c4ccccc34)c3sc4ccccc4c23)cc1 ; c1ccc(-c2ccc(-c3cc4c5cccc(N(c6ccc7ccccc7c6)c6ccc7ccccc7c6)c5sc4c4ccccc34)c3sc4ccccc4c23)cc1 ; c1ccc(N(c2ccccc2)c2cccc3c2sc2c4ccccc4c(-c4ccc5c(c4)C(c4ccccc4)(c4ccccc4)c4ccccc4-5)cc32)cc1]

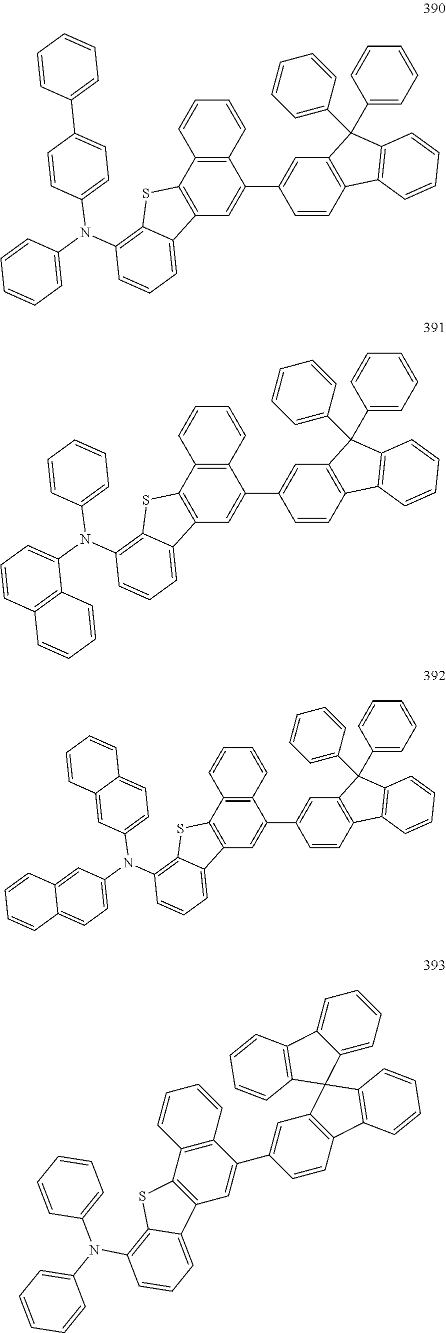 [SMILES: c1ccc(-c2ccc(N(c3ccccc3)c3cccc4c3sc3c5ccccc5c(-c5ccc6c(c5)C(c5ccccc5)(c5ccccc5)c5ccccc5-6)cc43)cc2)cc1 ; c1ccc(N(c2cccc3ccccc23)c2cccc3c2sc2c4ccccc4c(-c4ccc5c(c4)C(c4ccccc4)(c4ccccc4)c4ccccc4-5)cc32)cc1 ; c1ccc(C2(c3ccccc3)c3ccccc3-c3ccc(-c4cc5c6cccc(N(c7ccc8ccccc8c7)c7ccc8ccccc8c7)c6sc5c5ccccc45)cc32)cc1 ; c1ccc(N(c2ccccc2)c2cccc3c2sc2c4ccccc4c(-c4ccc5c(c4)C4(c6ccccc6-c6ccccc64)c4ccccc4-5)cc32)cc1]

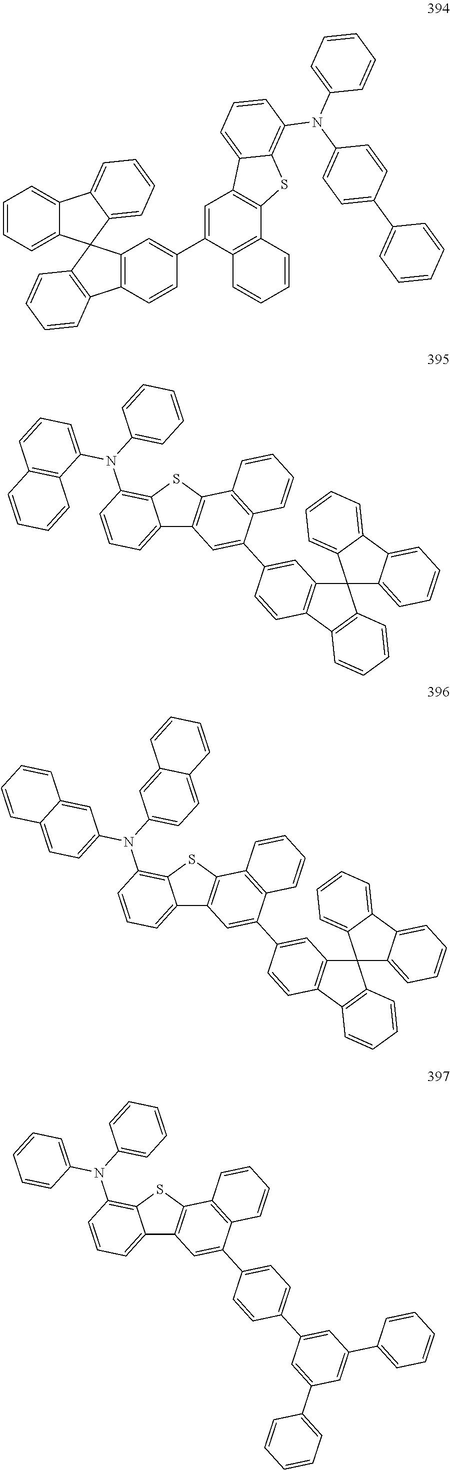 [SMILES: c1ccc(-c2ccc(N(c3ccccc3)c3cccc4c3sc3c5ccccc5c(-c5ccc6c(c5)C5(c7ccccc7-c7ccccc75)c5ccccc5-6)cc43)cc2)cc1 ; c1ccc(N(c2cccc3ccccc23)c2cccc3c2sc2c4ccccc4c(-c4ccc5c(c4)C4(c6ccccc6-c6ccccc64)c4ccccc4-5)cc32)cc1 ; c1ccc2c(c1)-c1ccccc1C21c2ccccc2-c2ccc(-c3cc4c5cccc(N(c6ccc7ccccc7c6)c6ccc7ccccc7c6)c5sc4c4ccccc34)cc21 ; c1ccc(-c2cc(-c3ccccc3)cc(-c3ccc(-c4cc5c6cccc(N(c7ccccc7)c7ccccc7)c6sc5c5ccccc45)cc3)c2)cc1]

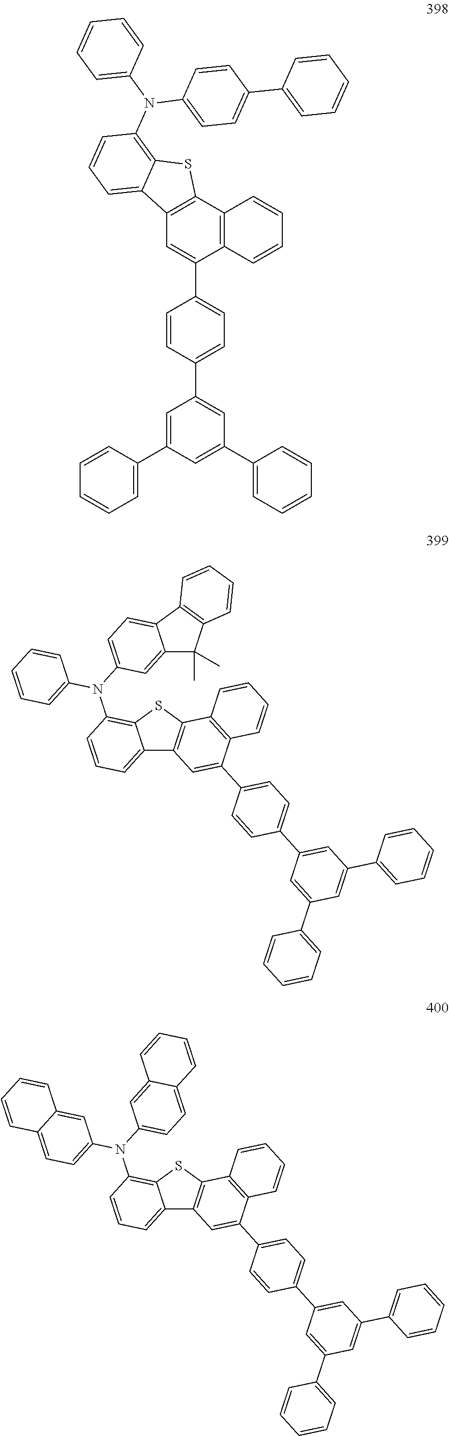 [SMILES: c1ccc(-c2ccc(N(c3ccccc3)c3cccc4c3sc3c5ccccc5c(-c5ccc(-c6cc(-c7ccccc7)cc(-c7ccccc7)c6)cc5)cc43)cc2)cc1 ; CC1(C)c2ccccc2-c2ccc(N(c3ccccc3)c3cccc4c3sc3c5ccccc5c(-c5ccc(-c6cc(-c7ccccc7)cc(-c7ccccc7)c6)cc5)cc43)cc21 ; c1ccc(-c2cc(-c3ccccc3)cc(-c3ccc(-c4cc5c6cccc(N(c7ccc8ccccc8c7)c7ccc8ccccc8c7)c6sc5c5ccccc45)cc3)c2)cc1]

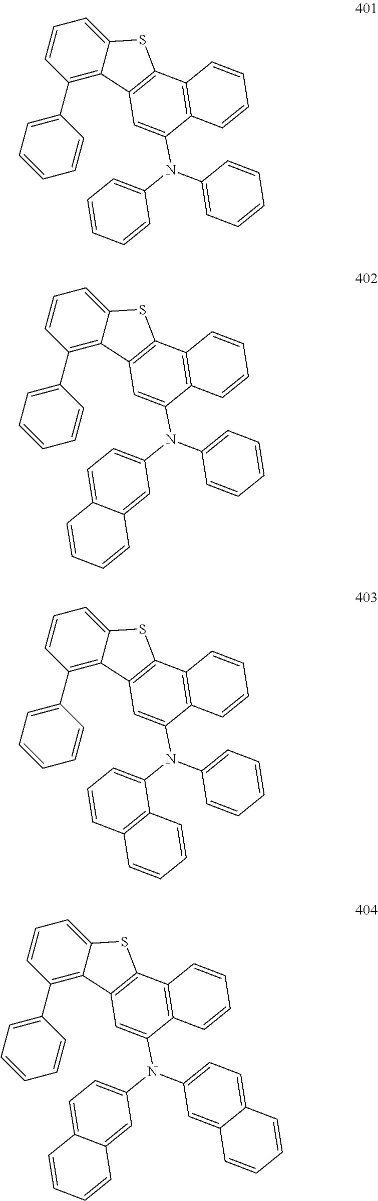

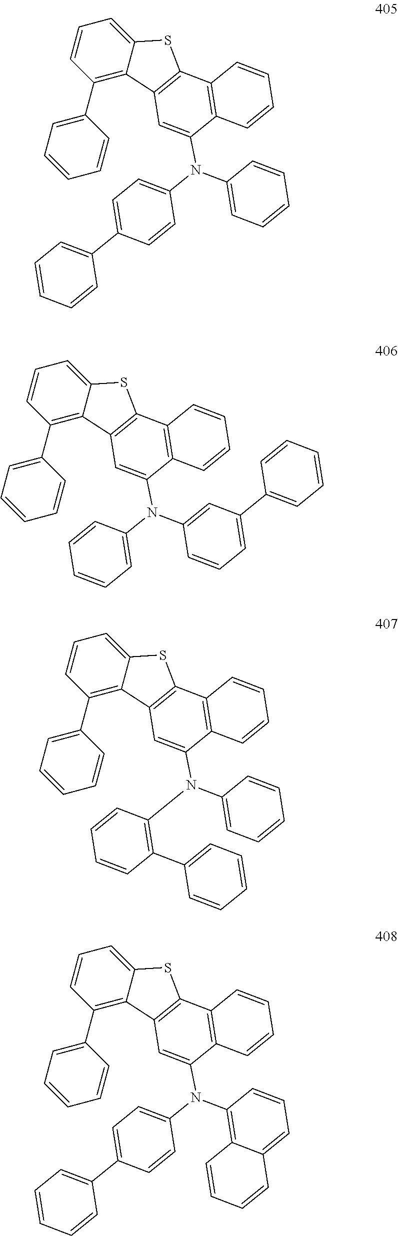

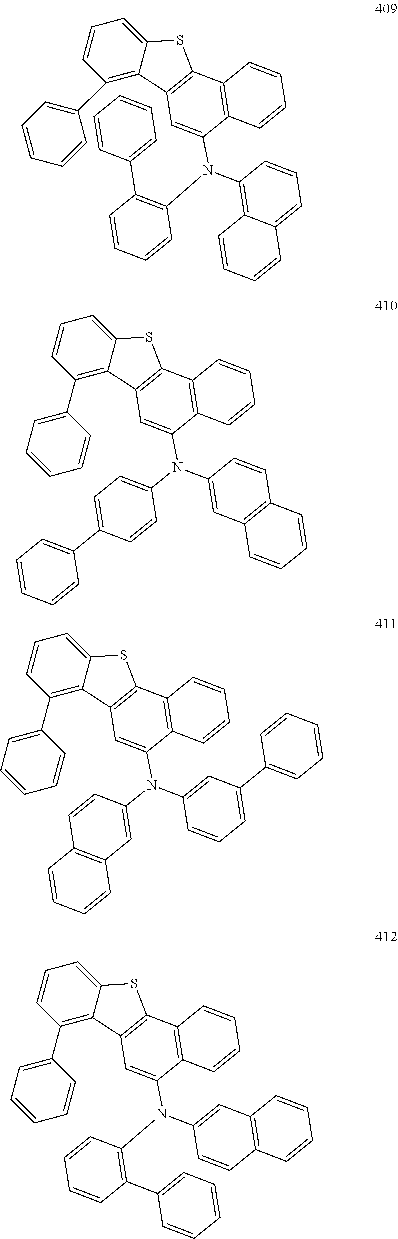

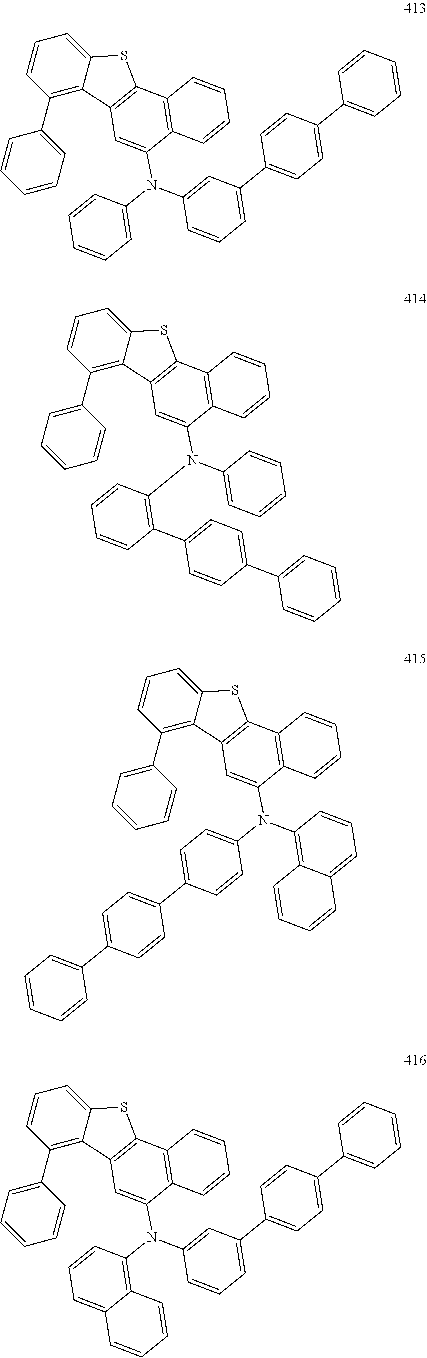 [SMILES: c1ccc(-c2ccc(-c3cccc(N(c4ccccc4)c4cc5c(sc6cccc(-c7ccccc7)c65)c5ccccc45)c3)cc2)cc1 ; c1ccc(-c2ccc(-c3ccccc3N(c3ccccc3)c3cc4c(sc5cccc(-c6ccccc6)c54)c4ccccc34)cc2)cc1 ; c1ccc(-c2ccc(-c3ccc(N(c4cccc5ccccc45)c4cc5c(sc6cccc(-c7ccccc7)c65)c5ccccc45)cc3)cc2)cc1 ; c1ccc(-c2ccc(-c3cccc(N(c4cccc5ccccc45)c4cc5c(sc6cccc(-c7ccccc7)c65)c5ccccc45)c3)cc2)cc1]

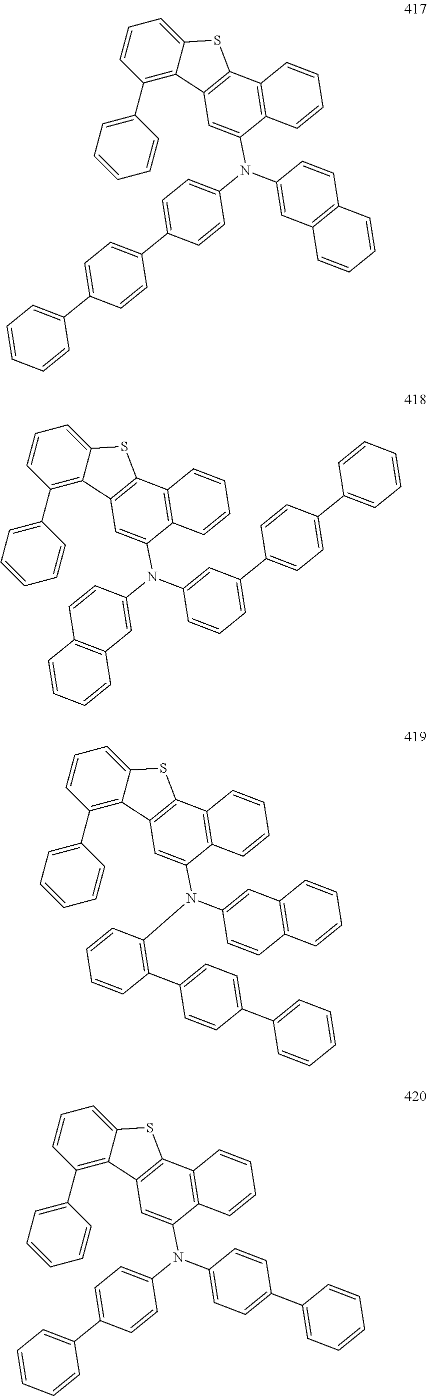

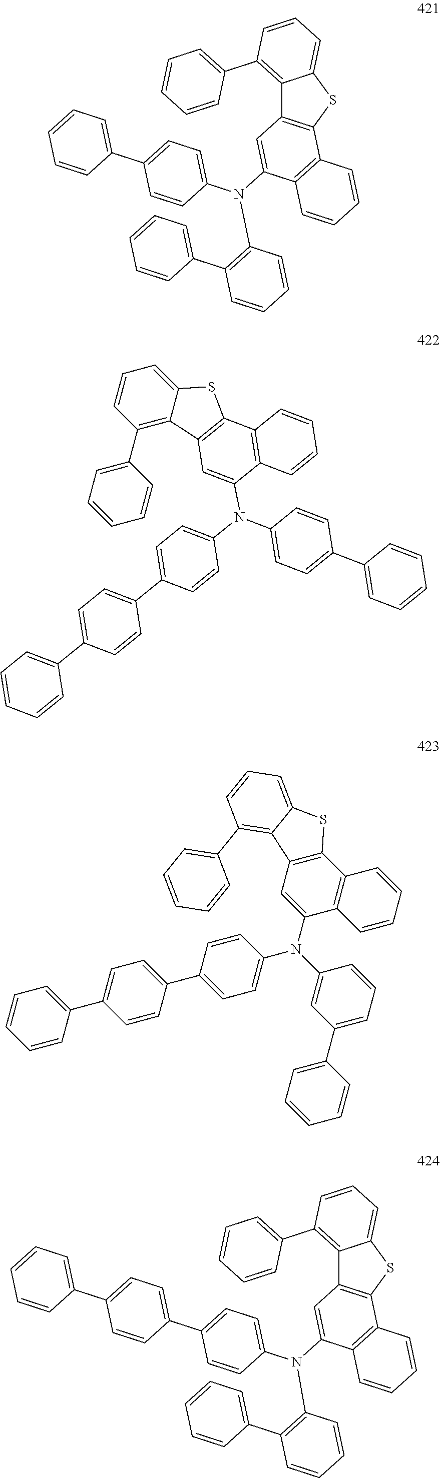 [SMILES: c1ccc(-c2ccc(N(c3ccccc3-c3ccccc3)c3cc4c(sc5cccc(-c6ccccc6)c54)c4ccccc34)cc2)cc1 ; c1ccc(-c2ccc(-c3ccc(N(c4ccc(-c5ccccc5)cc4)c4cc5c(sc6cccc(-c7ccccc7)c65)c5ccccc45)cc3)cc2)cc1 ; c1ccc(-c2ccc(-c3ccc(N(c4cccc(-c5ccccc5)c4)c4cc5c(sc6cccc(-c7ccccc7)c65)c5ccccc45)cc3)cc2)cc1 ; c1ccc(-c2ccc(-c3ccc(N(c4ccccc4-c4ccccc4)c4cc5c(sc6cccc(-c7ccccc7)c65)c5ccccc45)cc3)cc2)cc1]

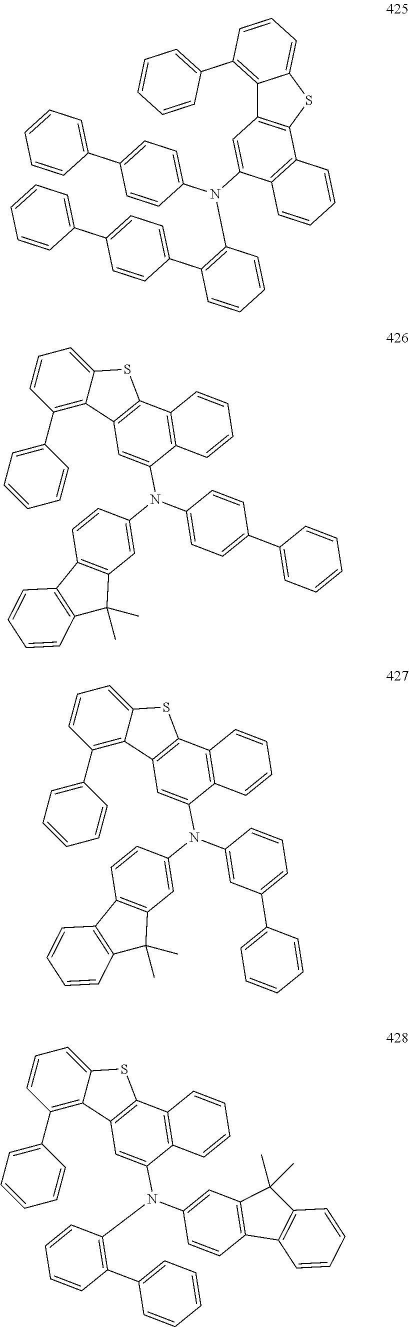

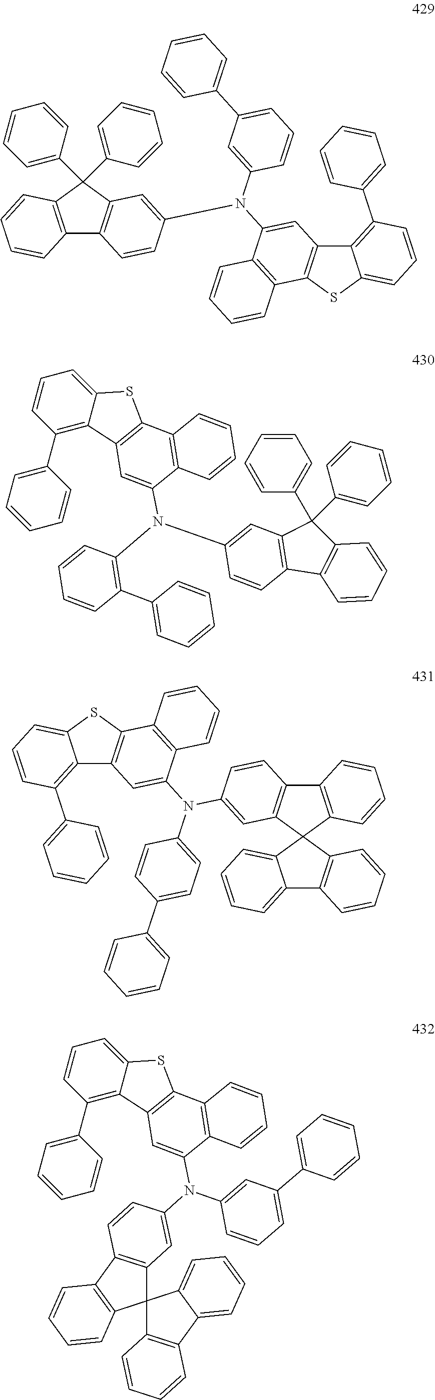

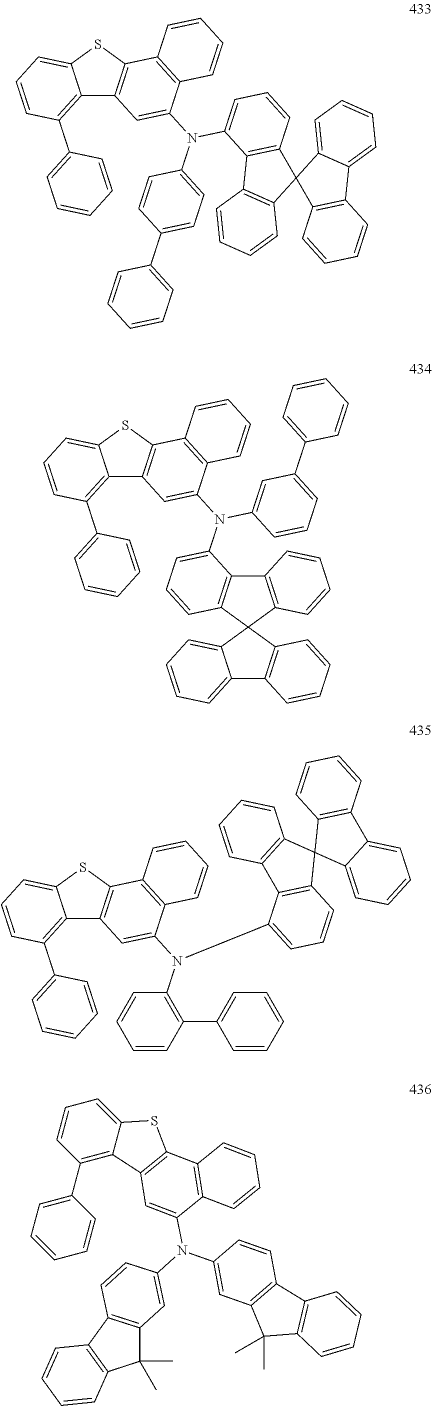 [SMILES: c1ccc(-c2ccc(N(c3cccc4c3-c3ccccc3C43c4ccccc4-c4ccccc43)c3cc4c(sc5cccc(-c6ccccc6)c54)c4ccccc34)cc2)cc1 ; c1ccc(-c2cccc(N(c3cccc4c3-c3ccccc3C43c4ccccc4-c4ccccc43)c3cc4c(sc5cccc(-c6ccccc6)c54)c4ccccc34)c2)cc1 ; c1ccc(-c2ccccc2N(c2cccc3c2-c2ccccc2C32c3ccccc3-c3ccccc32)c2cc3c(sc4cccc(-c5ccccc5)c43)c3ccccc23)cc1 ; CC1(C)c2ccccc2-c2ccc(N(c3ccc4c(c3)C(C)(C)c3ccccc3-4)c3cc4c(sc5cccc(-c6ccccc6)c54)c4ccccc34)cc21]

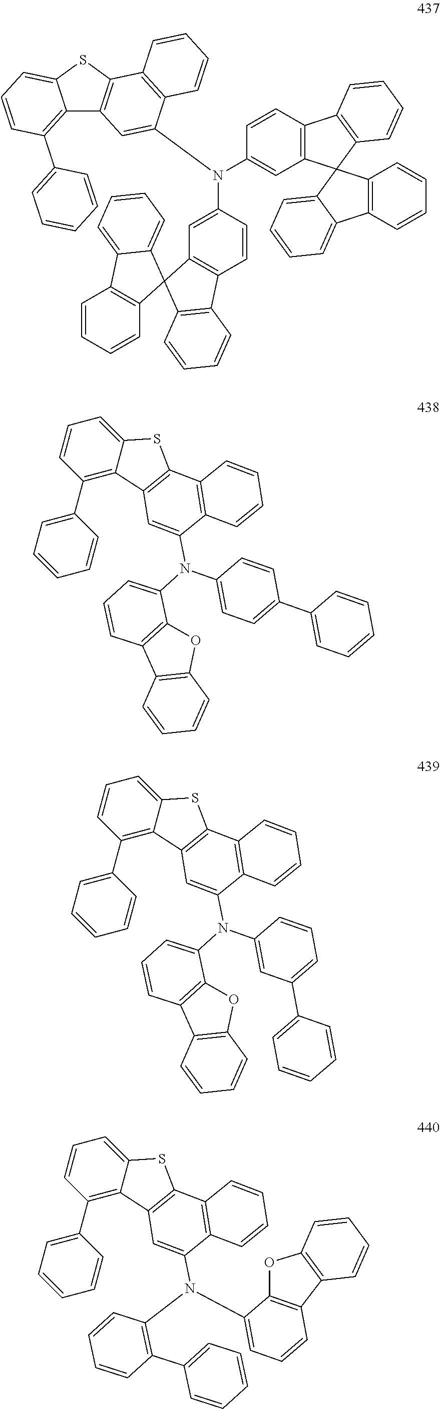 [SMILES: c1ccc(-c2cccc3sc4c5ccccc5c(N(c5ccc6c(c5)C5(c7ccccc7-c7ccccc75)c5ccccc5-6)c5ccc6c(c5)C5(c7ccccc7-c7ccccc75)c5ccccc5-6)cc4c23)cc1 ; c1ccc(-c2ccc(N(c3cc4c(sc5cccc(-c6ccccc6)c54)c4ccccc34)c3cccc4c3oc3ccccc34)cc2)cc1 ; c1ccc(-c2cccc(N(c3cc4c(sc5cccc(-c6ccccc6)c54)c4ccccc34)c3cccc4c3oc3ccccc34)c2)cc1 ; c1ccc(-c2ccccc2N(c2cc3c(sc4cccc(-c5ccccc5)c43)c3ccccc23)c2cccc3c2oc2ccccc23)cc1]

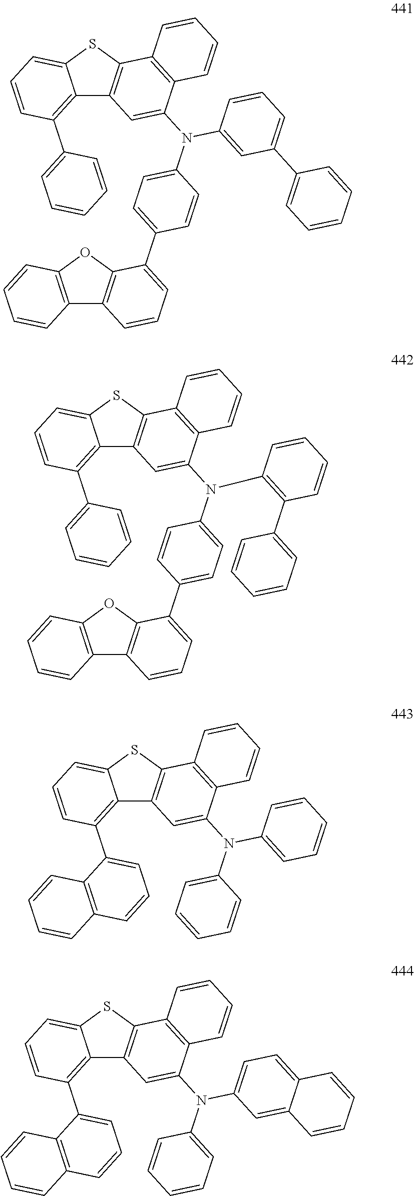

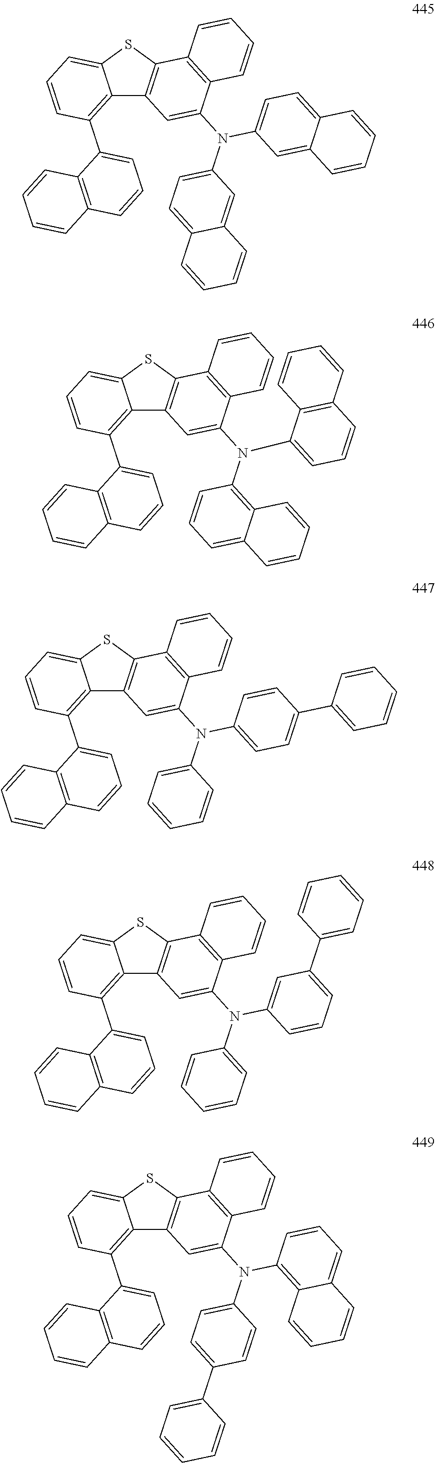 [SMILES: c1ccc2cc(N(c3ccc4ccccc4c3)c3cc4c(sc5cccc(-c6cccc7ccccc67)c54)c4ccccc34)ccc2c1 ; c1ccc2c(-c3cccc4sc5c6ccccc6c(N(c6cccc7ccccc67)c6cccc7ccccc67)cc5c34)cccc2c1 ; c1ccc(-c2ccc(N(c3ccccc3)c3cc4c(sc5cccc(-c6cccc7ccccc67)c54)c4ccccc34)cc2)cc1 ; c1ccc(-c2cccc(N(c3ccccc3)c3cc4c(sc5cccc(-c6cccc7ccccc67)c54)c4ccccc34)c2)cc1 ; c1ccc(-c2ccc(N(c3cccc4ccccc34)c3cc4c(sc5cccc(-c6cccc7ccccc67)c54)c4ccccc34)cc2)cc1]

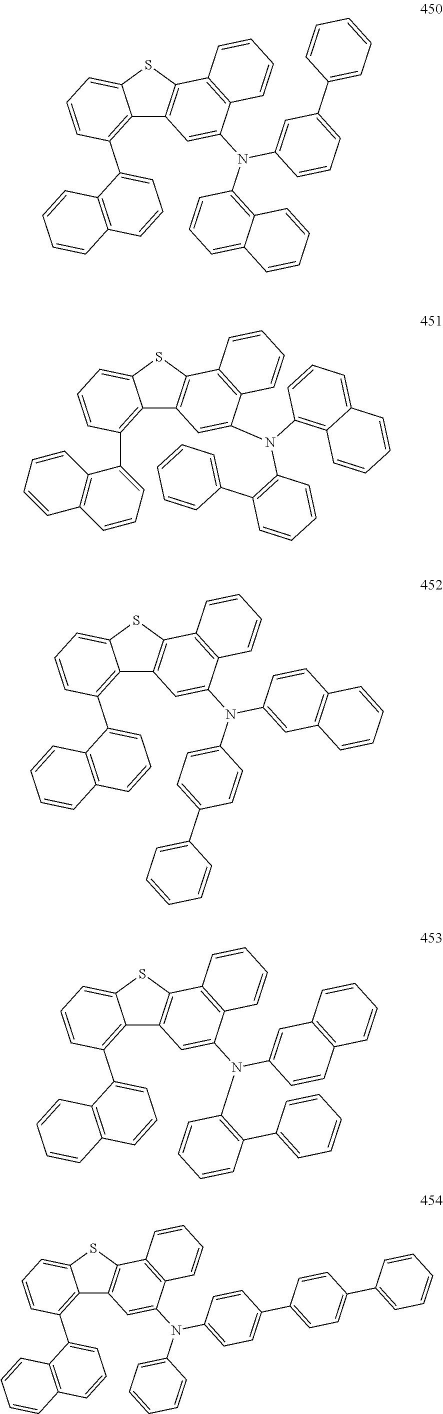 [SMILES: c1ccc(-c2cccc(N(c3cccc4ccccc34)c3cc4c(sc5cccc(-c6cccc7ccccc67)c54)c4ccccc34)c2)cc1 ; c1ccc(-c2ccccc2N(c2cccc3ccccc23)c2cc3c(sc4cccc(-c5cccc6ccccc56)c43)c3ccccc23)cc1 ; c1ccc(-c2ccc(N(c3ccc4ccccc4c3)c3cc4c(sc5cccc(-c6cccc7ccccc67)c54)c4ccccc34)cc2)cc1 ; c1ccc(-c2ccccc2N(c2ccc3ccccc3c2)c2cc3c(sc4cccc(-c5cccc6ccccc56)c43)c3ccccc23)cc1 ; c1ccc(-c2ccc(-c3ccc(N(c4ccccc4)c4cc5c(sc6cccc(-c7cccc8ccccc78)c65)c5ccccc45)cc3)cc2)cc1]

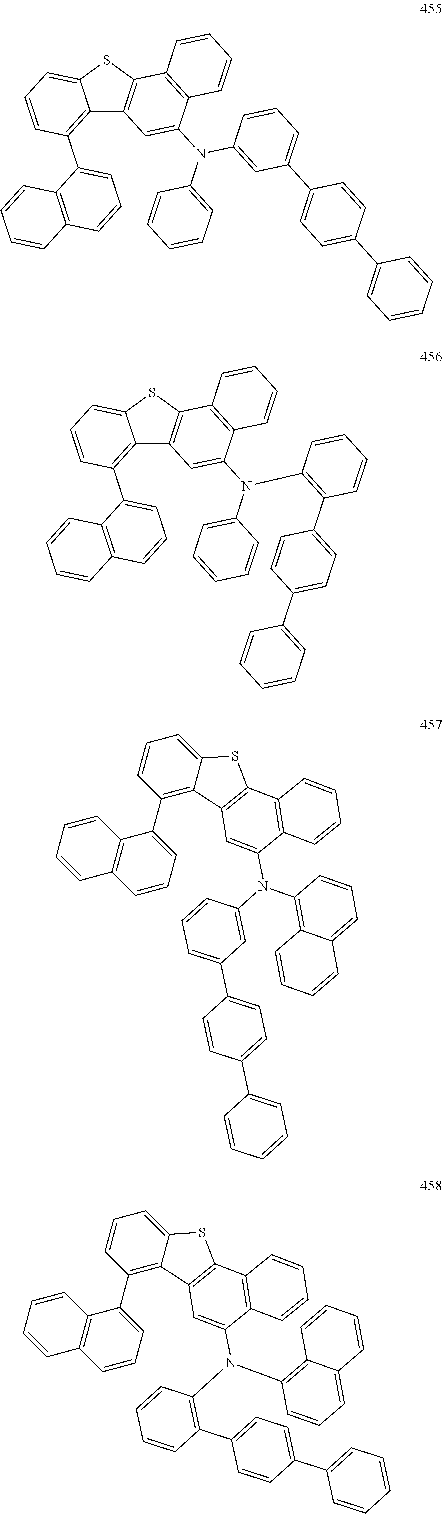 [SMILES: c1ccc(-c2ccc(-c3cccc(N(c4ccccc4)c4cc5c(sc6cccc(-c7cccc8ccccc78)c65)c5ccccc45)c3)cc2)cc1 ; c1ccc(-c2ccc(-c3ccccc3N(c3ccccc3)c3cc4c(sc5cccc(-c6cccc7ccccc67)c54)c4ccccc34)cc2)cc1 ; c1ccc(-c2ccc(-c3cccc(N(c4cccc5ccccc45)c4cc5c(sc6cccc(-c7cccc8ccccc78)c65)c5ccccc45)c3)cc2)cc1 ; c1ccc(-c2ccc(-c3ccccc3N(c3cccc4ccccc34)c3cc4c(sc5cccc(-c6cccc7ccccc67)c54)c4ccccc34)cc2)cc1]

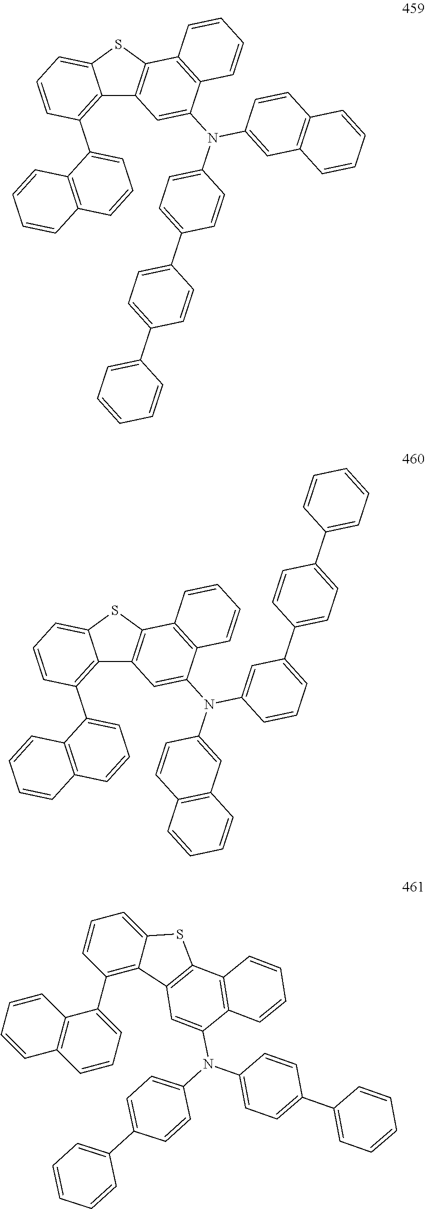 [SMILES: c1ccc(-c2ccc(-c3ccc(N(c4ccc5ccccc5c4)c4cc5c(sc6cccc(-c7cccc8ccccc78)c65)c5ccccc45)cc3)cc2)cc1 ; c1ccc(-c2ccc(-c3cccc(N(c4ccc5ccccc5c4)c4cc5c(sc6cccc(-c7cccc8ccccc78)c65)c5ccccc45)c3)cc2)cc1 ; c1ccc(-c2ccc(N(c3ccc(-c4ccccc4)cc3)c3cc4c(sc5cccc(-c6cccc7ccccc67)c54)c4ccccc34)cc2)cc1]

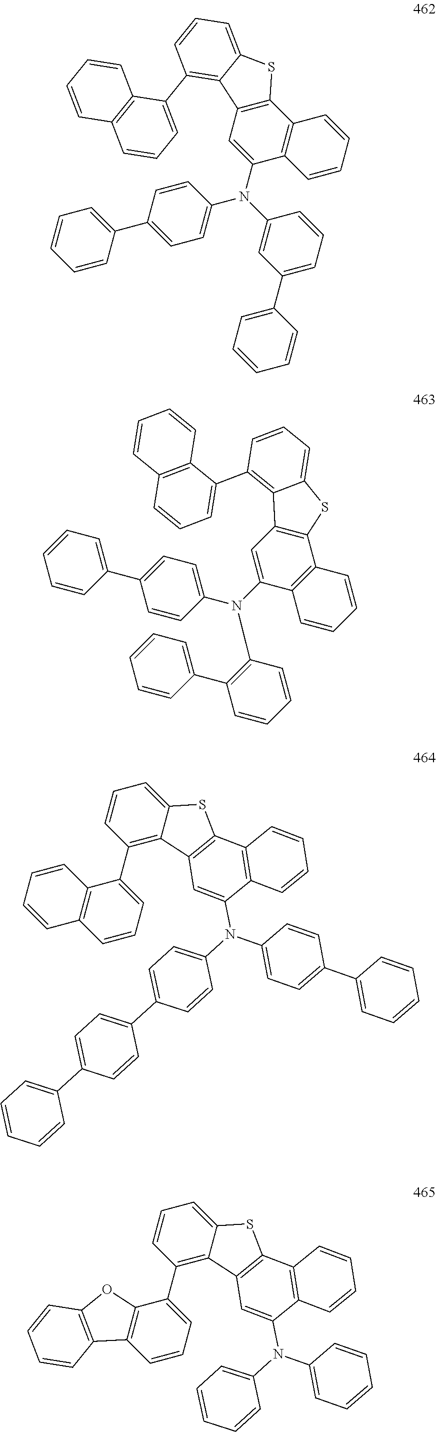 [SMILES: c1ccc(-c2ccc(N(c3cccc(-c4ccccc4)c3)c3cc4c(sc5cccc(-c6cccc7ccccc67)c54)c4ccccc34)cc2)cc1 ; c1ccc(-c2ccc(N(c3ccccc3-c3ccccc3)c3cc4c(sc5cccc(-c6cccc7ccccc67)c54)c4ccccc34)cc2)cc1 ; c1ccc(-c2ccc(-c3ccc(N(c4ccc(-c5ccccc5)cc4)c4cc5c(sc6cccc(-c7cccc8ccccc78)c65)c5ccccc45)cc3)cc2)cc1 ; c1ccc(N(c2ccccc2)c2cc3c(sc4cccc(-c5cccc6c5oc5ccccc56)c43)c3ccccc23)cc1]

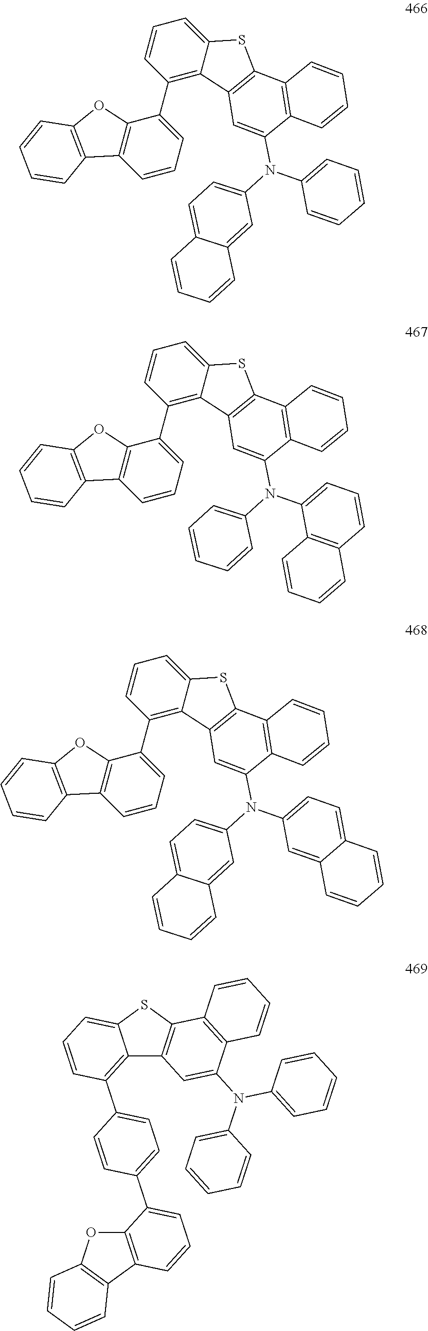

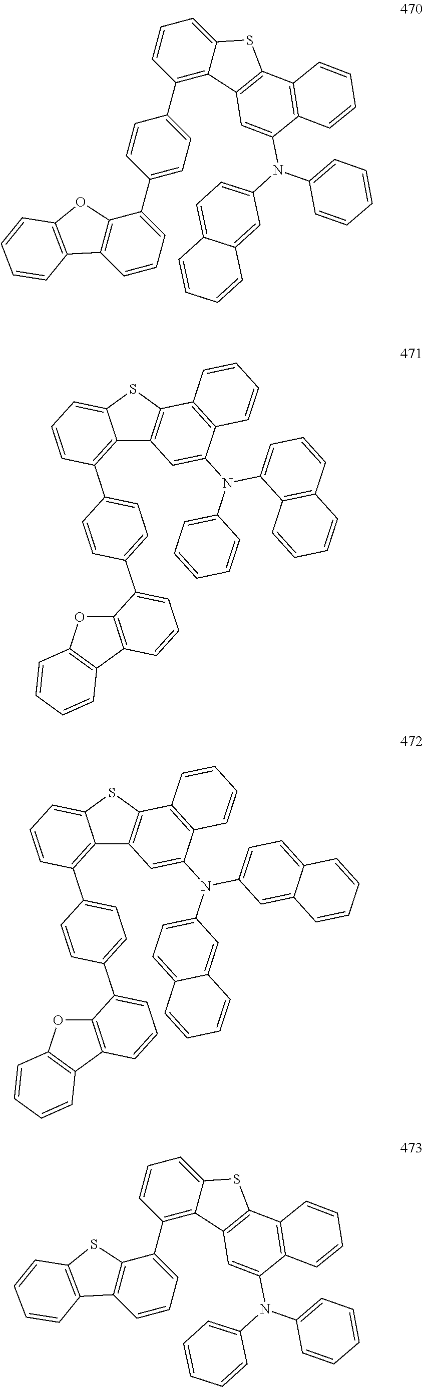 [SMILES: c1ccc(N(c2ccc3ccccc3c2)c2cc3c(sc4cccc(-c5ccc(-c6cccc7c6oc6ccccc67)cc5)c43)c3ccccc23)cc1 ; c1ccc(N(c2cccc3ccccc23)c2cc3c(sc4cccc(-c5ccc(-c6cccc7c6oc6ccccc67)cc5)c43)c3ccccc23)cc1 ; c1ccc2cc(N(c3ccc4ccccc4c3)c3cc4c(sc5cccc(-c6ccc(-c7cccc8c7oc7ccccc78)cc6)c54)c4ccccc34)ccc2c1 ; c1ccc(N(c2ccccc2)c2cc3c(sc4cccc(-c5cccc6c5sc5ccccc56)c43)c3ccccc23)cc1]

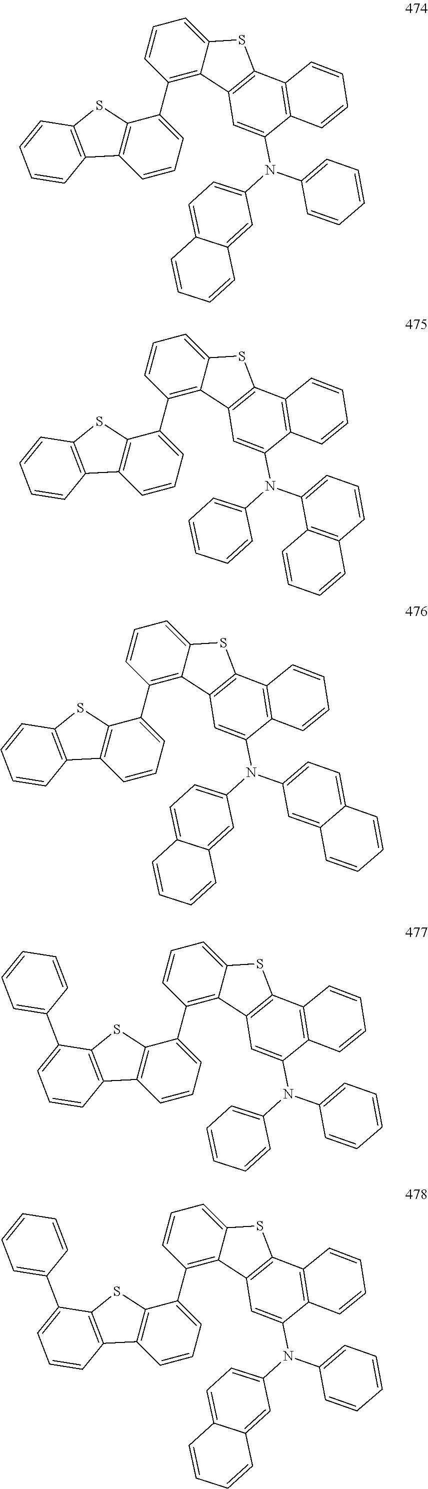

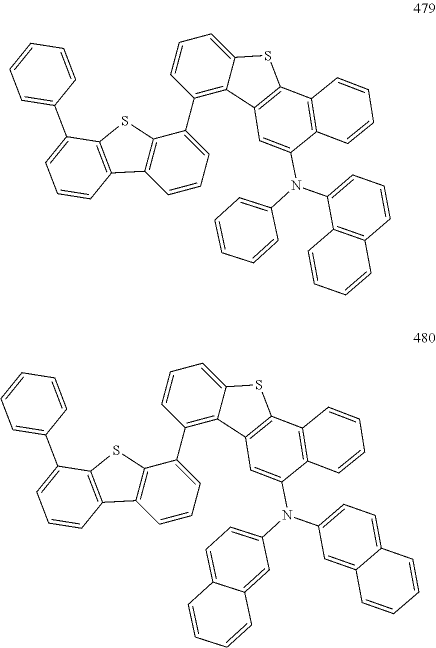 [SMILES: c1ccc(-c2cccc3c2sc2c(-c4cccc5sc6c7ccccc7c(N(c7ccccc7)c7cccc8ccccc78)cc6c45)cccc23)cc1 ; c1ccc(-c2cccc3c2sc2c(-c4cccc5sc6c7ccccc7c(N(c7ccc8ccccc8c7)c7ccc8ccccc8c7)cc6c45)cccc23)cc1]

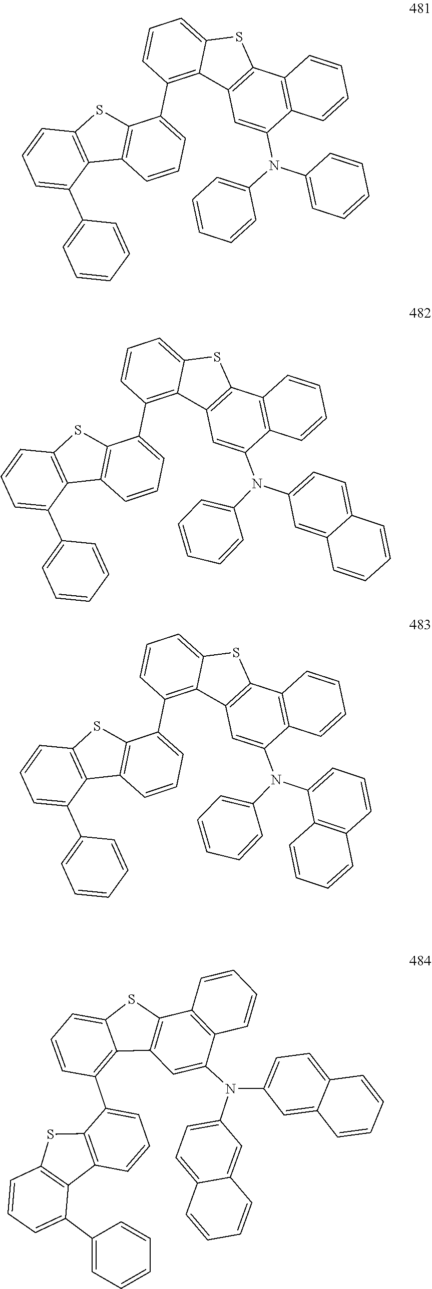 [SMILES: c1ccc(-c2cccc3sc4c(-c5cccc6sc7c8ccccc8c(N(c8ccccc8)c8ccccc8)cc7c56)cccc4c23)cc1 ; c1ccc(-c2cccc3sc4c(-c5cccc6sc7c8ccccc8c(N(c8ccccc8)c8ccc9ccccc9c8)cc7c56)cccc4c23)cc1 ; c1ccc(-c2cccc3sc4c(-c5cccc6sc7c8ccccc8c(N(c8ccccc8)c8cccc9ccccc89)cc7c56)cccc4c23)cc1 ; c1ccc(-c2cccc3sc4c(-c5cccc6sc7c8ccccc8c(N(c8ccc9ccccc9c8)c8ccc9ccccc9c8)cc7c56)cccc4c23)cc1]

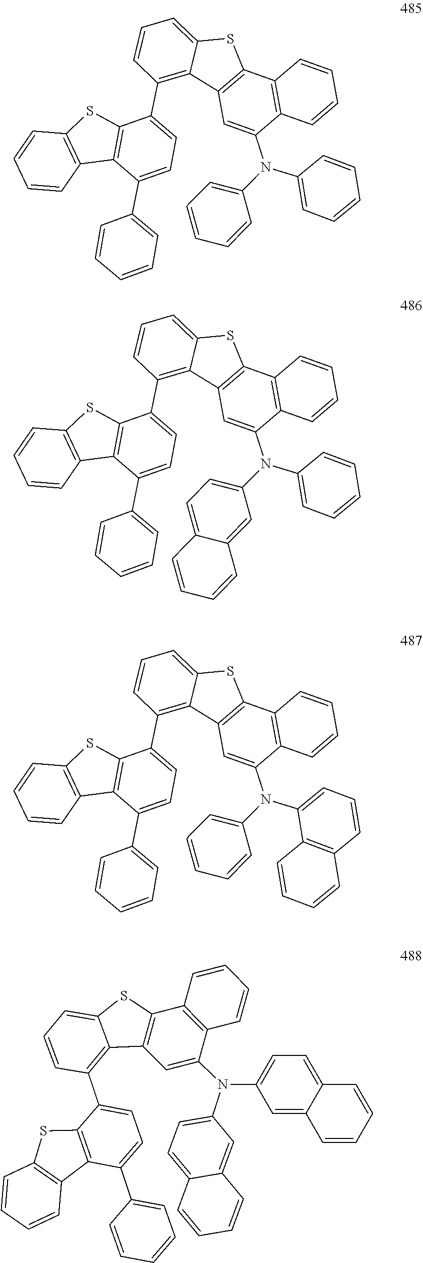 [SMILES: c1ccc(-c2ccc(-c3cccc4sc5c6ccccc6c(N(c6ccccc6)c6ccccc6)cc5c34)c3sc4ccccc4c23)cc1 ; c1ccc(-c2ccc(-c3cccc4sc5c6ccccc6c(N(c6ccccc6)c6ccc7ccccc7c6)cc5c34)c3sc4ccccc4c23)cc1 ; c1ccc(-c2ccc(-c3cccc4sc5c6ccccc6c(N(c6ccccc6)c6cccc7ccccc67)cc5c34)c3sc4ccccc4c23)cc1 ; c1ccc(-c2ccc(-c3cccc4sc5c6ccccc6c(N(c6ccc7ccccc7c6)c6ccc7ccccc7c6)cc5c34)c3sc4ccccc4c23)cc1]

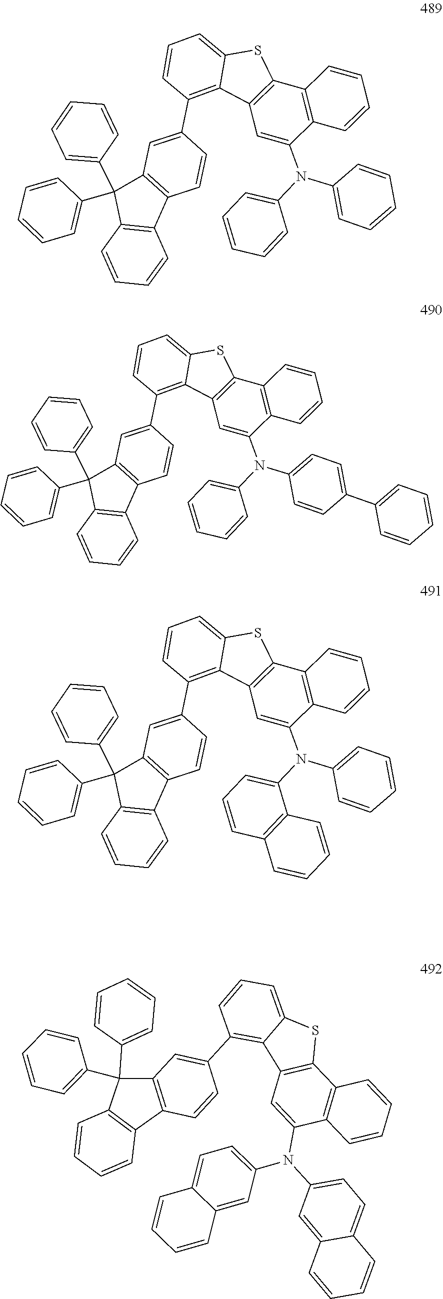 [SMILES: c1ccc(N(c2ccccc2)c2cc3c(sc4cccc(-c5ccc6c(c5)C(c5ccccc5)(c5ccccc5)c5ccccc5-6)c43)c3ccccc23)cc1 ; c1ccc(-c2ccc(N(c3ccccc3)c3cc4c(sc5cccc(-c6ccc7c(c6)C(c6ccccc6)(c6ccccc6)c6ccccc6-7)c54)c4ccccc34)cc2)cc1 ; c1ccc(N(c2cccc3ccccc23)c2cc3c(sc4cccc(-c5ccc6c(c5)C(c5ccccc5)(c5ccccc5)c5ccccc5-6)c43)c3ccccc23)cc1 ; c1ccc(C2(c3ccccc3)c3ccccc3-c3ccc(-c4cccc5sc6c7ccccc7c(N(c7ccc8ccccc8c7)c7ccc8ccccc8c7)cc6c45)cc32)cc1]

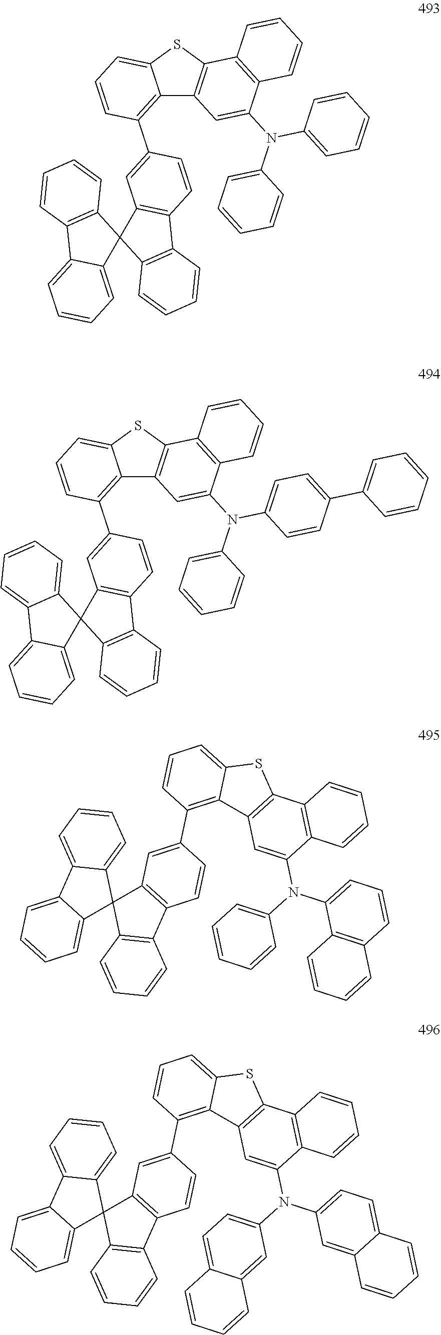 [SMILES: c1ccc(N(c2ccccc2)c2cc3c(sc4cccc(-c5ccc6c(c5)C5(c7ccccc7-c7ccccc75)c5ccccc5-6)c43)c3ccccc23)cc1 ; c1ccc(-c2ccc(N(c3ccccc3)c3cc4c(sc5cccc(-c6ccc7c(c6)C6(c8ccccc8-c8ccccc86)c6ccccc6-7)c54)c4ccccc34)cc2)cc1 ; c1ccc(N(c2cccc3ccccc23)c2cc3c(sc4cccc(-c5ccc6c(c5)C5(c7ccccc7-c7ccccc75)c5ccccc5-6)c43)c3ccccc23)cc1 ; c1ccc2c(c1)-c1ccccc1C21c2ccccc2-c2ccc(-c3cccc4sc5c6ccccc6c(N(c6ccc7ccccc7c6)c6ccc7ccccc7c6)cc5c34)cc21]

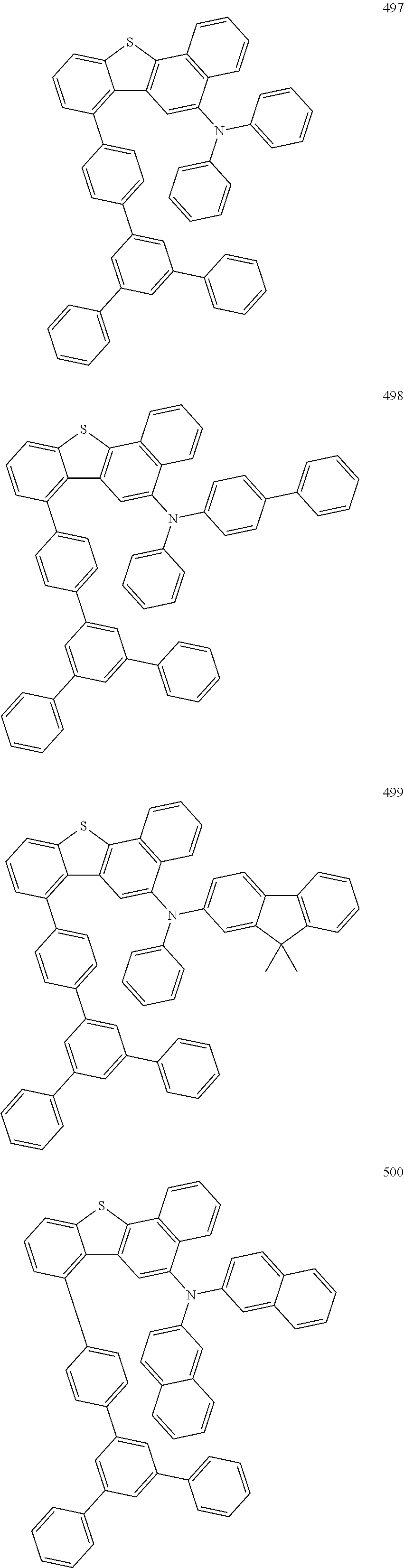 [SMILES: c1ccc(-c2cc(-c3ccccc3)cc(-c3ccc(-c4cccc5sc6c7ccccc7c(N(c7ccccc7)c7ccccc7)cc6c45)cc3)c2)cc1 ; c1ccc(-c2ccc(N(c3ccccc3)c3cc4c(sc5cccc(-c6ccc(-c7cc(-c8ccccc8)cc(-c8ccccc8)c7)cc6)c54)c4ccccc34)cc2)cc1 ; CC1(C)c2ccccc2-c2ccc(N(c3ccccc3)c3cc4c(sc5cccc(-c6ccc(-c7cc(-c8ccccc8)cc(-c8ccccc8)c7)cc6)c54)c4ccccc34)cc21 ; c1ccc(-c2cc(-c3ccccc3)cc(-c3ccc(-c4cccc5sc6c7ccccc7c(N(c7ccc8ccccc8c7)c7ccc8ccccc8c7)cc6c45)cc3)c2)cc1]

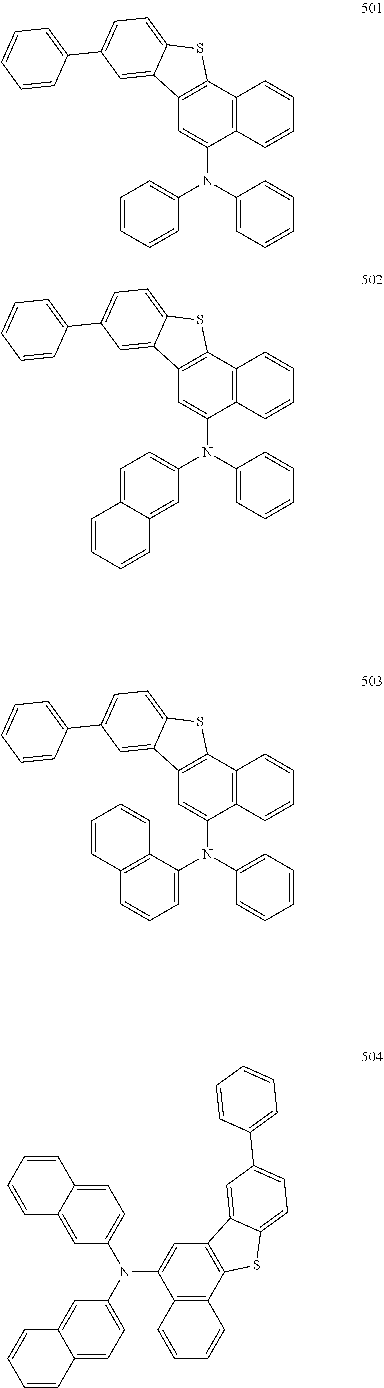

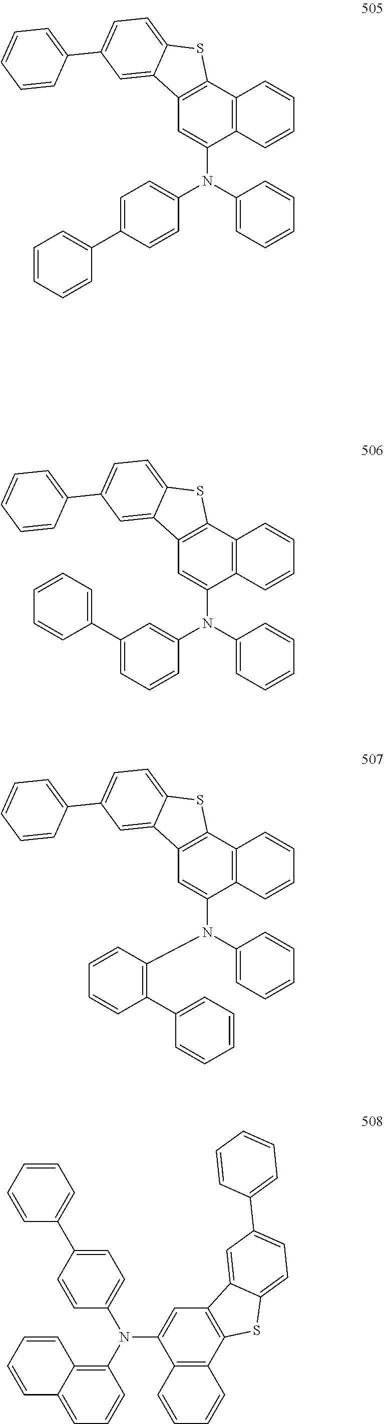 [SMILES: c1ccc(-c2ccc(N(c3ccccc3)c3cc4c5cc(-c6ccccc6)ccc5sc4c4ccccc34)cc2)cc1 ; c1ccc(-c2cccc(N(c3ccccc3)c3cc4c5cc(-c6ccccc6)ccc5sc4c4ccccc34)c2)cc1 ; c1ccc(-c2ccc3sc4c5ccccc5c(N(c5ccccc5)c5ccccc5-c5ccccc5)cc4c3c2)cc1 ; c1ccc(-c2ccc(N(c3cccc4ccccc34)c3cc4c5cc(-c6ccccc6)ccc5sc4c4ccccc34)cc2)cc1]

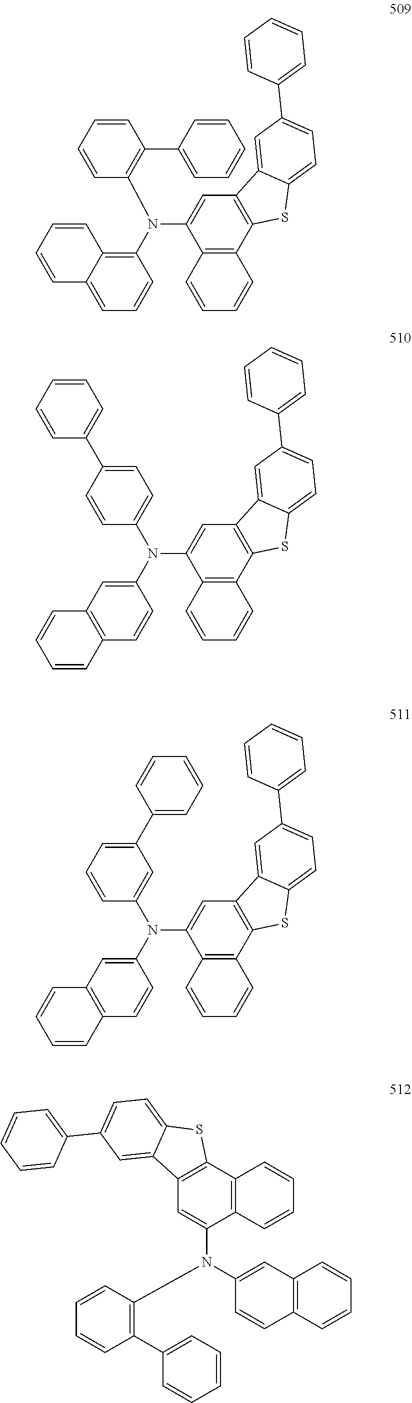 [SMILES: c1ccc(-c2ccc3sc4c5ccccc5c(N(c5ccccc5-c5ccccc5)c5cccc6ccccc56)cc4c3c2)cc1 ; c1ccc(-c2ccc(N(c3ccc4ccccc4c3)c3cc4c5cc(-c6ccccc6)ccc5sc4c4ccccc34)cc2)cc1 ; c1ccc(-c2cccc(N(c3ccc4ccccc4c3)c3cc4c5cc(-c6ccccc6)ccc5sc4c4ccccc34)c2)cc1 ; c1ccc(-c2ccc3sc4c5ccccc5c(N(c5ccc6ccccc6c5)c5ccccc5-c5ccccc5)cc4c3c2)cc1]

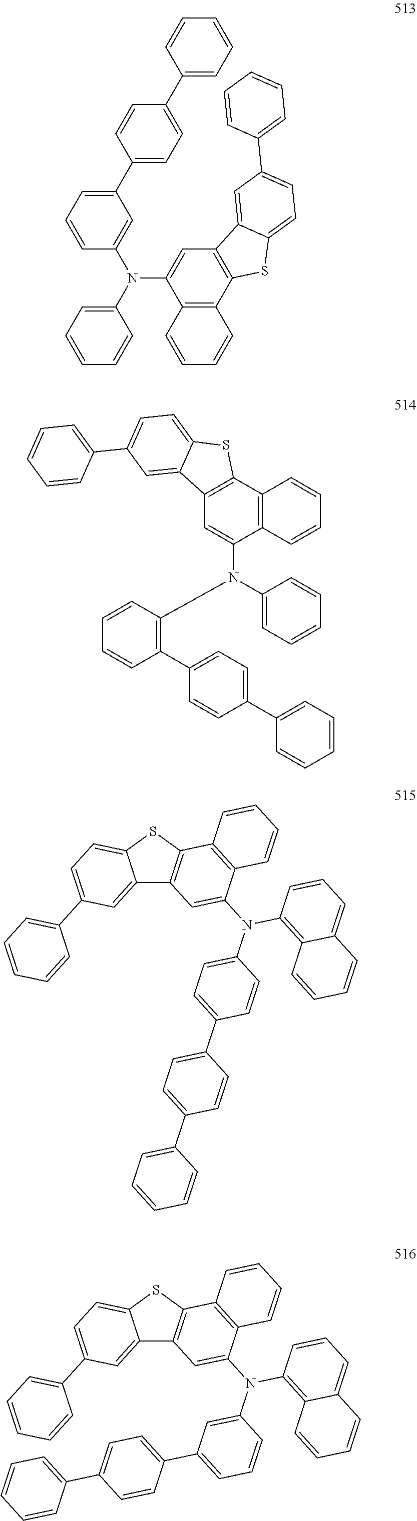 [SMILES: c1ccc(-c2ccc(-c3cccc(N(c4ccccc4)c4cc5c6cc(-c7ccccc7)ccc6sc5c5ccccc45)c3)cc2)cc1 ; c1ccc(-c2ccc(-c3ccccc3N(c3ccccc3)c3cc4c5cc(-c6ccccc6)ccc5sc4c4ccccc34)cc2)cc1 ; c1ccc(-c2ccc(-c3ccc(N(c4cccc5ccccc45)c4cc5c6cc(-c7ccccc7)ccc6sc5c5ccccc45)cc3)cc2)cc1 ; c1ccc(-c2ccc(-c3cccc(N(c4cccc5ccccc45)c4cc5c6cc(-c7ccccc7)ccc6sc5c5ccccc45)c3)cc2)cc1]

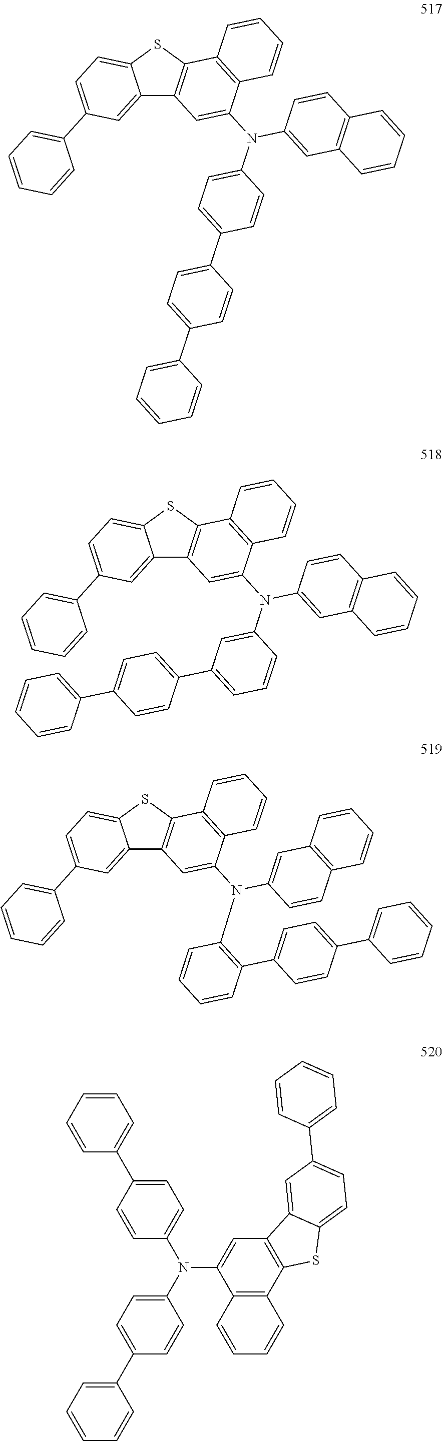 [SMILES: c1ccc(-c2ccc(-c3ccc(N(c4ccc5ccccc5c4)c4cc5c6cc(-c7ccccc7)ccc6sc5c5ccccc45)cc3)cc2)cc1 ; c1ccc(-c2ccc(-c3cccc(N(c4ccc5ccccc5c4)c4cc5c6cc(-c7ccccc7)ccc6sc5c5ccccc45)c3)cc2)cc1 ; c1ccc(-c2ccc(-c3ccccc3N(c3ccc4ccccc4c3)c3cc4c5cc(-c6ccccc6)ccc5sc4c4ccccc34)cc2)cc1 ; c1ccc(-c2ccc(N(c3ccc(-c4ccccc4)cc3)c3cc4c5cc(-c6ccccc6)ccc5sc4c4ccccc34)cc2)cc1]

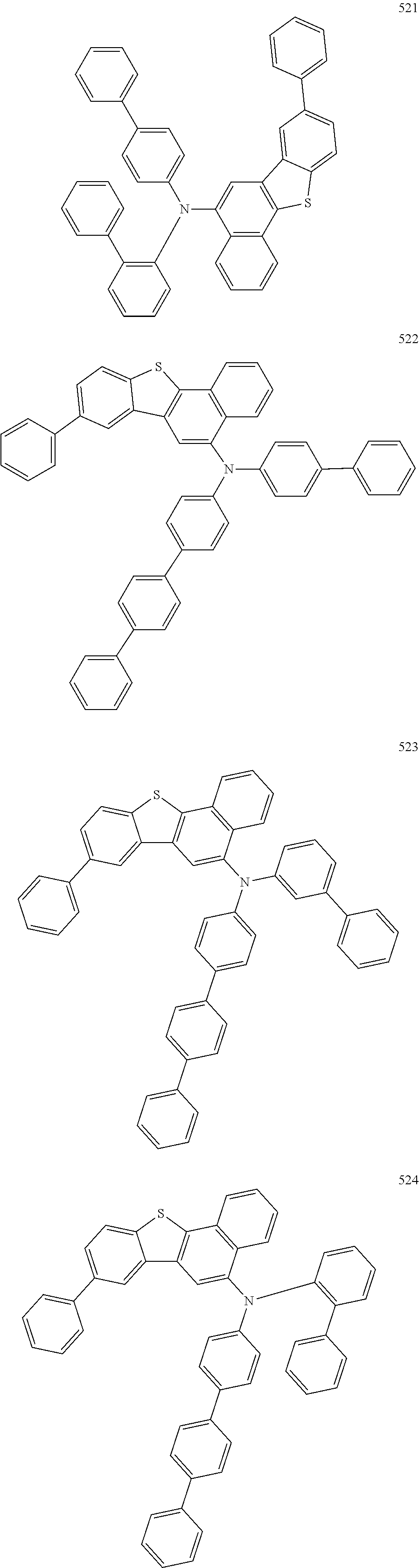 [SMILES: c1ccc(-c2ccc(N(c3ccccc3-c3ccccc3)c3cc4c5cc(-c6ccccc6)ccc5sc4c4ccccc34)cc2)cc1 ; c1ccc(-c2ccc(-c3ccc(N(c4ccc(-c5ccccc5)cc4)c4cc5c6cc(-c7ccccc7)ccc6sc5c5ccccc45)cc3)cc2)cc1 ; c1ccc(-c2ccc(-c3ccc(N(c4cccc(-c5ccccc5)c4)c4cc5c6cc(-c7ccccc7)ccc6sc5c5ccccc45)cc3)cc2)cc1 ; c1ccc(-c2ccc(-c3ccc(N(c4ccccc4-c4ccccc4)c4cc5c6cc(-c7ccccc7)ccc6sc5c5ccccc45)cc3)cc2)cc1]

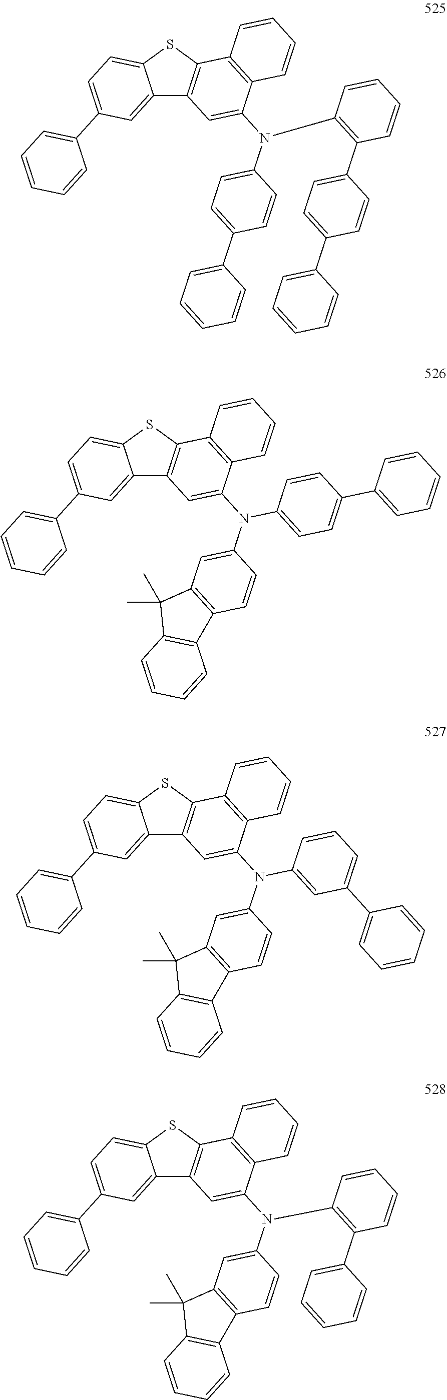 [SMILES: c1ccc(-c2ccc(-c3ccccc3N(c3ccc(-c4ccccc4)cc3)c3cc4c5cc(-c6ccccc6)ccc5sc4c4ccccc34)cc2)cc1 ; CC1(C)c2ccccc2-c2ccc(N(c3ccc(-c4ccccc4)cc3)c3cc4c5cc(-c6ccccc6)ccc5sc4c4ccccc34)cc21 ; CC1(C)c2ccccc2-c2ccc(N(c3cccc(-c4ccccc4)c3)c3cc4c5cc(-c6ccccc6)ccc5sc4c4ccccc34)cc21 ; CC1(C)c2ccccc2-c2ccc(N(c3ccccc3-c3ccccc3)c3cc4c5cc(-c6ccccc6)ccc5sc4c4ccccc34)cc21]

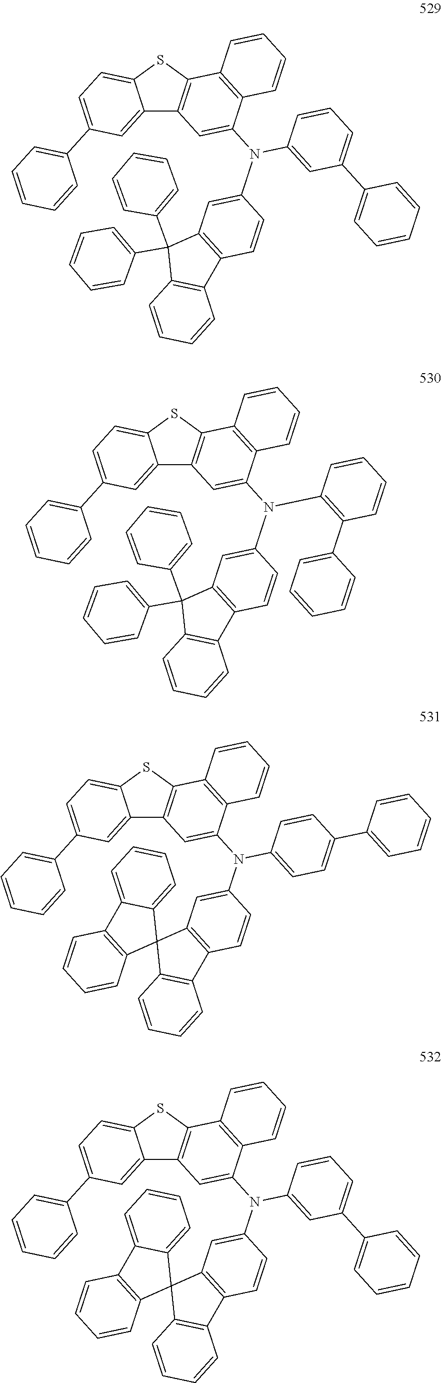 [SMILES: c1ccc(-c2cccc(N(c3ccc4c(c3)C(c3ccccc3)(c3ccccc3)c3ccccc3-4)c3cc4c5cc(-c6ccccc6)ccc5sc4c4ccccc34)c2)cc1 ; c1ccc(-c2ccc3sc4c5ccccc5c(N(c5ccc6c(c5)C(c5ccccc5)(c5ccccc5)c5ccccc5-6)c5ccccc5-c5ccccc5)cc4c3c2)cc1 ; c1ccc(-c2ccc(N(c3ccc4c(c3)C3(c5ccccc5-c5ccccc53)c3ccccc3-4)c3cc4c5cc(-c6ccccc6)ccc5sc4c4ccccc34)cc2)cc1 ; c1ccc(-c2cccc(N(c3ccc4c(c3)C3(c5ccccc5-c5ccccc53)c3ccccc3-4)c3cc4c5cc(-c6ccccc6)ccc5sc4c4ccccc34)c2)cc1]

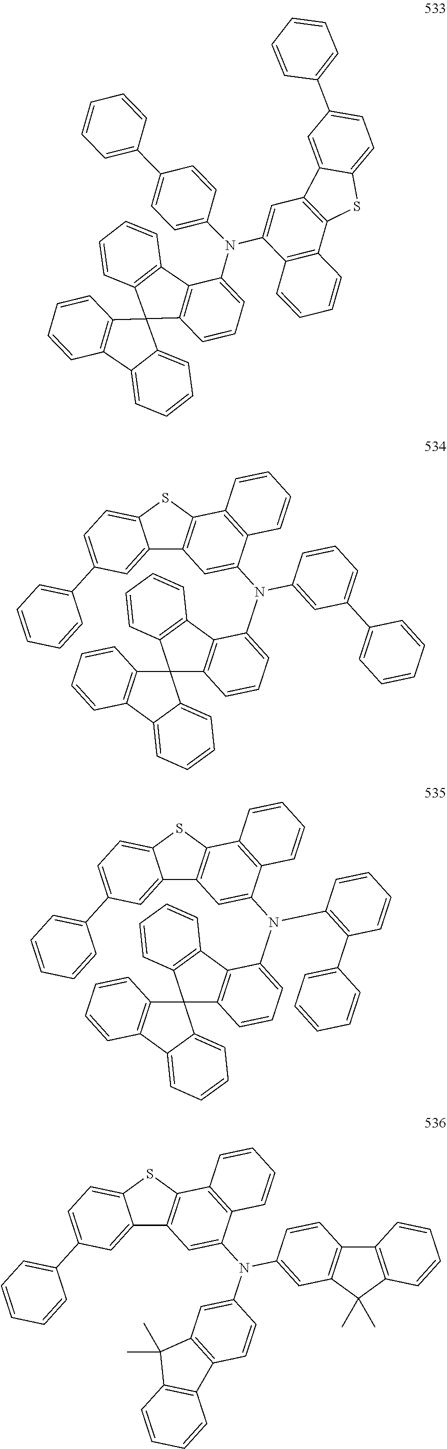 [SMILES: c1ccc(-c2ccc(N(c3cccc4c3-c3ccccc3C43c4ccccc4-c4ccccc43)c3cc4c5cc(-c6ccccc6)ccc5sc4c4ccccc34)cc2)cc1 ; c1ccc(-c2cccc(N(c3cccc4c3-c3ccccc3C43c4ccccc4-c4ccccc43)c3cc4c5cc(-c6ccccc6)ccc5sc4c4ccccc34)c2)cc1 ; c1ccc(-c2ccc3sc4c5ccccc5c(N(c5ccccc5-c5ccccc5)c5cccc6c5-c5ccccc5C65c6ccccc6-c6ccccc65)cc4c3c2)cc1 ; CC1(C)c2ccccc2-c2ccc(N(c3ccc4c(c3)C(C)(C)c3ccccc3-4)c3cc4c5cc(-c6ccccc6)ccc5sc4c4ccccc34)cc21]

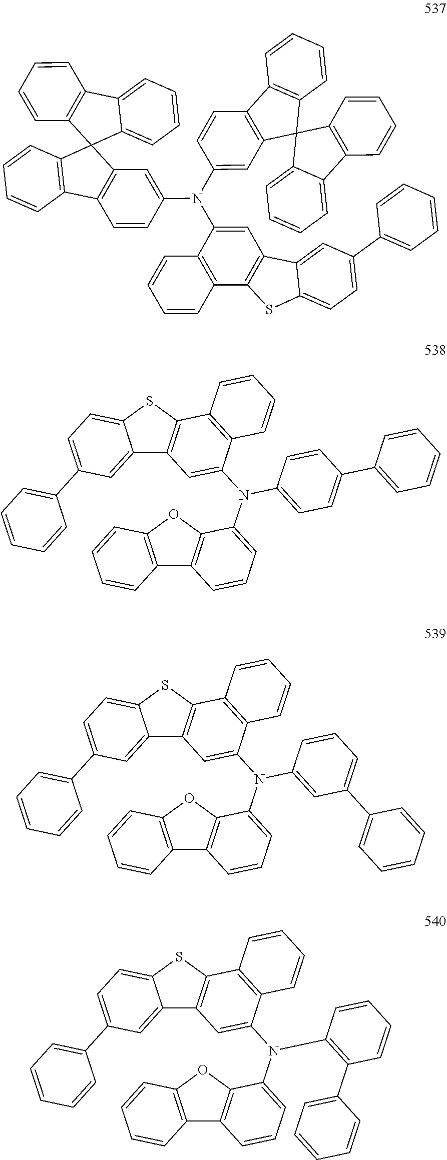 [SMILES: c1ccc(-c2ccc3sc4c5ccccc5c(N(c5ccc6c(c5)C5(c7ccccc7-c7ccccc75)c5ccccc5-6)c5ccc6c(c5)C5(c7ccccc7-c7ccccc75)c5ccccc5-6)cc4c3c2)cc1 ; c1ccc(-c2ccc(N(c3cc4c5cc(-c6ccccc6)ccc5sc4c4ccccc34)c3cccc4c3oc3ccccc34)cc2)cc1 ; c1ccc(-c2cccc(N(c3cc4c5cc(-c6ccccc6)ccc5sc4c4ccccc34)c3cccc4c3oc3ccccc34)c2)cc1 ; c1ccc(-c2ccc3sc4c5ccccc5c(N(c5ccccc5-c5ccccc5)c5cccc6c5oc5ccccc56)cc4c3c2)cc1]

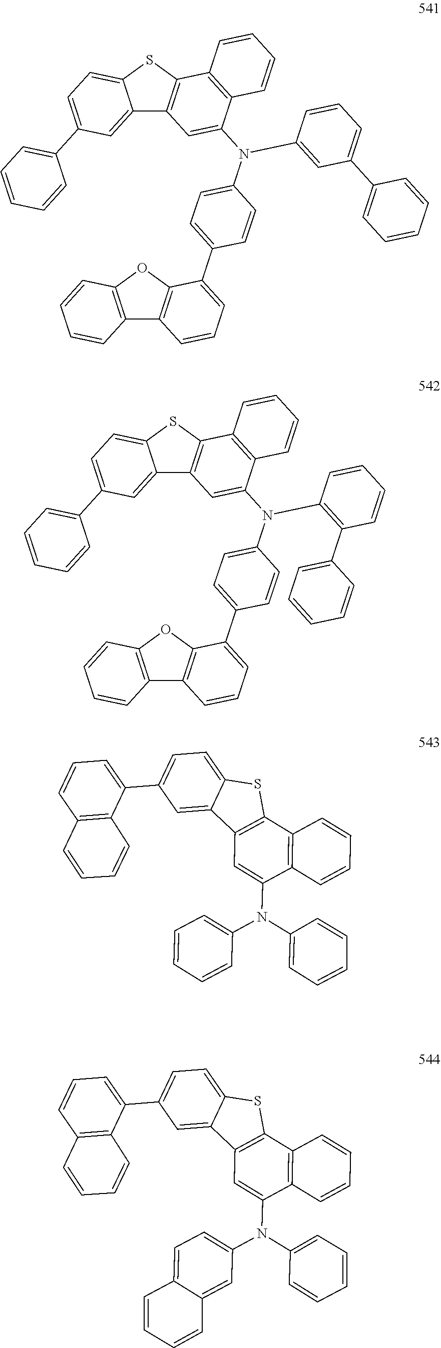 [SMILES: c1ccc(-c2cccc(N(c3ccc(-c4cccc5c4oc4ccccc45)cc3)c3cc4c5cc(-c6ccccc6)ccc5sc4c4ccccc34)c2)cc1 ; c1ccc(-c2ccc3sc4c5ccccc5c(N(c5ccc(-c6cccc7c6oc6ccccc67)cc5)c5ccccc5-c5ccccc5)cc4c3c2)cc1 ; c1ccc(N(c2ccccc2)c2cc3c4cc(-c5cccc6ccccc56)ccc4sc3c3ccccc23)cc1 ; c1ccc(N(c2ccc3ccccc3c2)c2cc3c4cc(-c5cccc6ccccc56)ccc4sc3c3ccccc23)cc1]

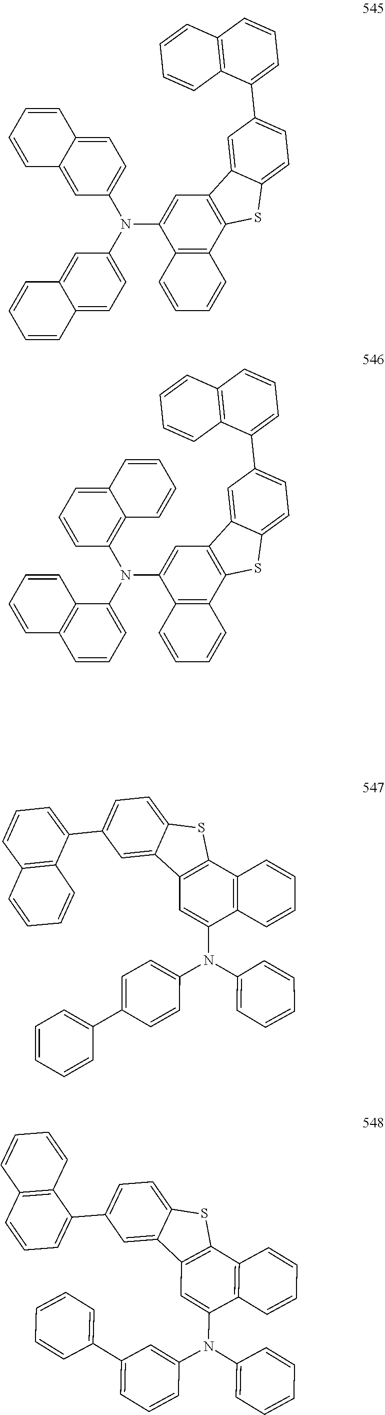 [SMILES: c1ccc2cc(N(c3ccc4ccccc4c3)c3cc4c5cc(-c6cccc7ccccc67)ccc5sc4c4ccccc34)ccc2c1 ; c1ccc2c(-c3ccc4sc5c6ccccc6c(N(c6cccc7ccccc67)c6cccc7ccccc67)cc5c4c3)cccc2c1 ; c1ccc(-c2ccc(N(c3ccccc3)c3cc4c5cc(-c6cccc7ccccc67)ccc5sc4c4ccccc34)cc2)cc1 ; c1ccc(-c2cccc(N(c3ccccc3)c3cc4c5cc(-c6cccc7ccccc67)ccc5sc4c4ccccc34)c2)cc1]

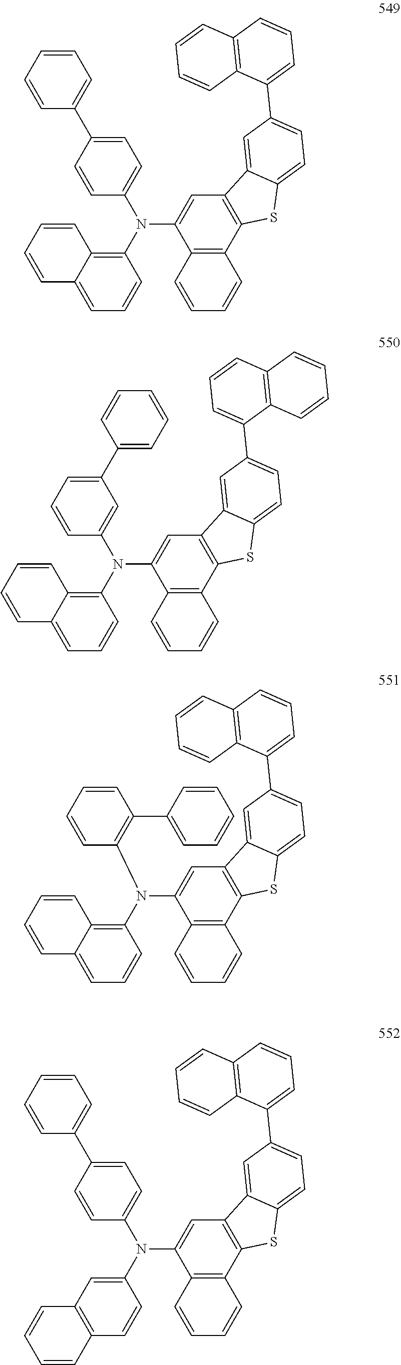

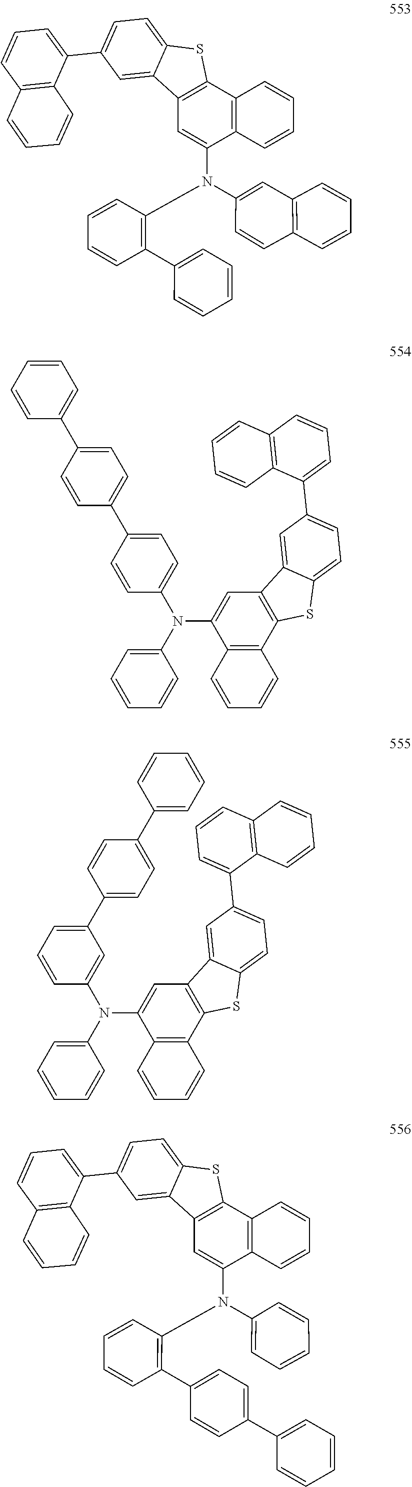

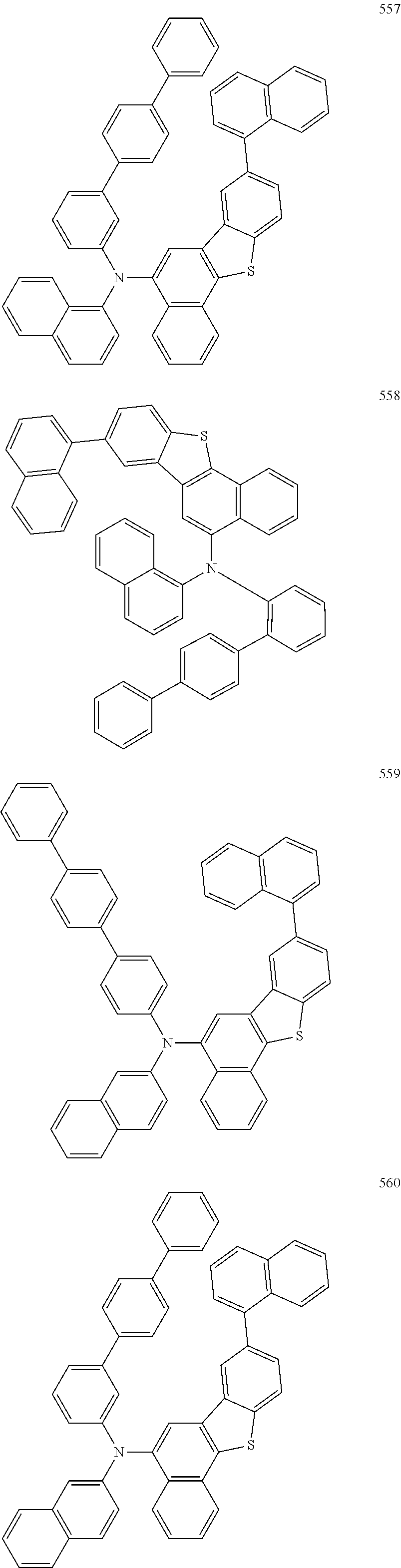

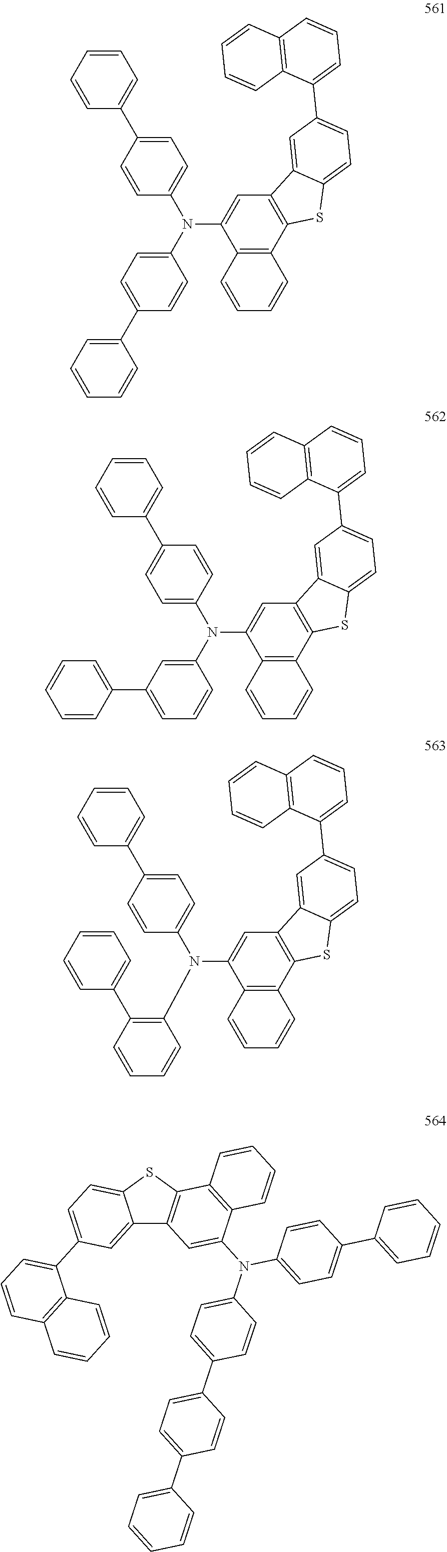

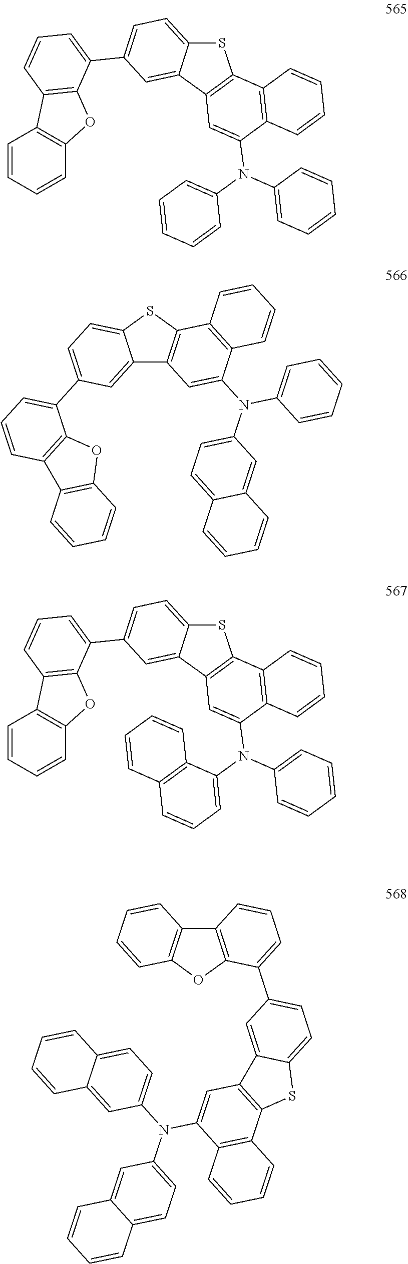 [SMILES: c1ccc(N(c2ccccc2)c2cc3c4cc(-c5cccc6c5oc5ccccc56)ccc4sc3c3ccccc23)cc1 ; c1ccc(N(c2ccc3ccccc3c2)c2cc3c4cc(-c5cccc6c5oc5ccccc56)ccc4sc3c3ccccc23)cc1 ; c1ccc(N(c2cccc3ccccc23)c2cc3c4cc(-c5cccc6c5oc5ccccc56)ccc4sc3c3ccccc23)cc1 ; c1ccc2cc(N(c3ccc4ccccc4c3)c3cc4c5cc(-c6cccc7c6oc6ccccc67)ccc5sc4c4ccccc34)ccc2c1]

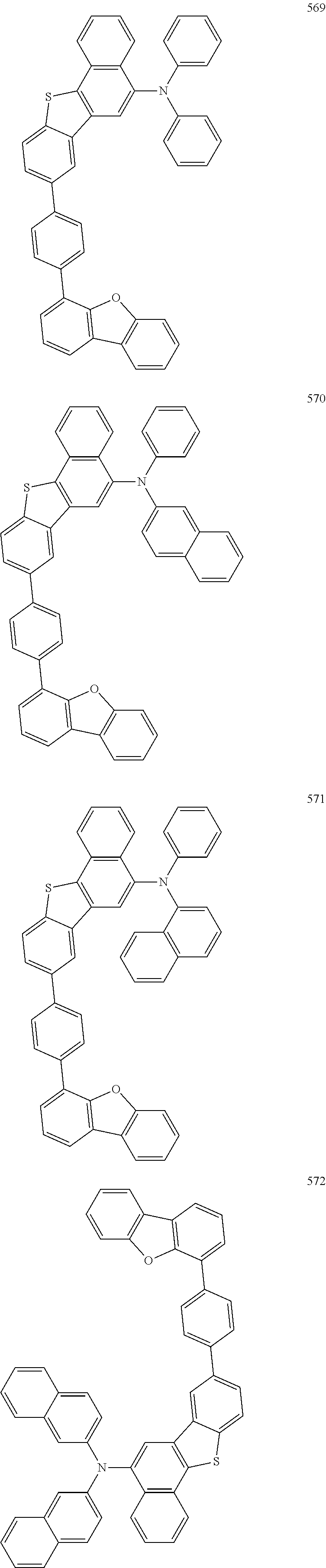

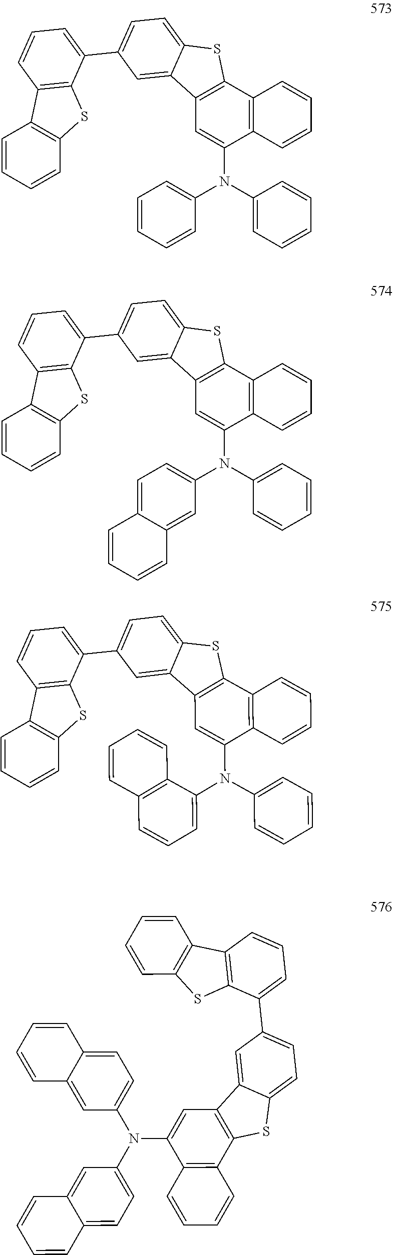

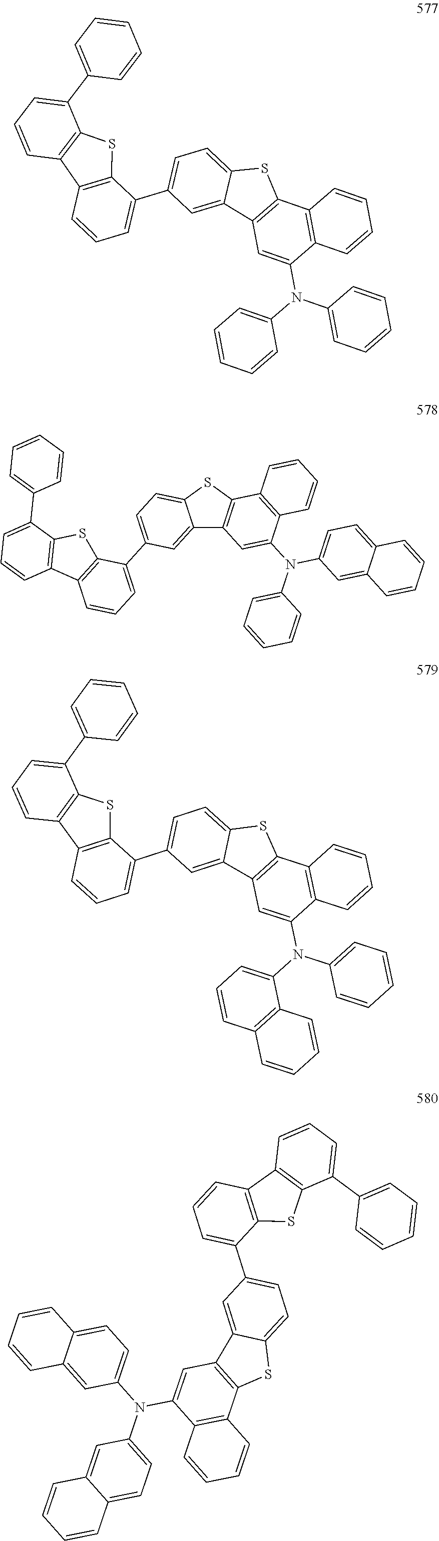 [SMILES: c1ccc(-c2cccc3c2sc2c(-c4ccc5sc6c7ccccc7c(N(c7ccccc7)c7ccccc7)cc6c5c4)cccc23)cc1 ; c1ccc(-c2cccc3c2sc2c(-c4ccc5sc6c7ccccc7c(N(c7ccccc7)c7ccc8ccccc8c7)cc6c5c4)cccc23)cc1 ; c1ccc(-c2cccc3c2sc2c(-c4ccc5sc6c7ccccc7c(N(c7ccccc7)c7cccc8ccccc78)cc6c5c4)cccc23)cc1 ; c1ccc(-c2cccc3c2sc2c(-c4ccc5sc6c7ccccc7c(N(c7ccc8ccccc8c7)c7ccc8ccccc8c7)cc6c5c4)cccc23)cc1]

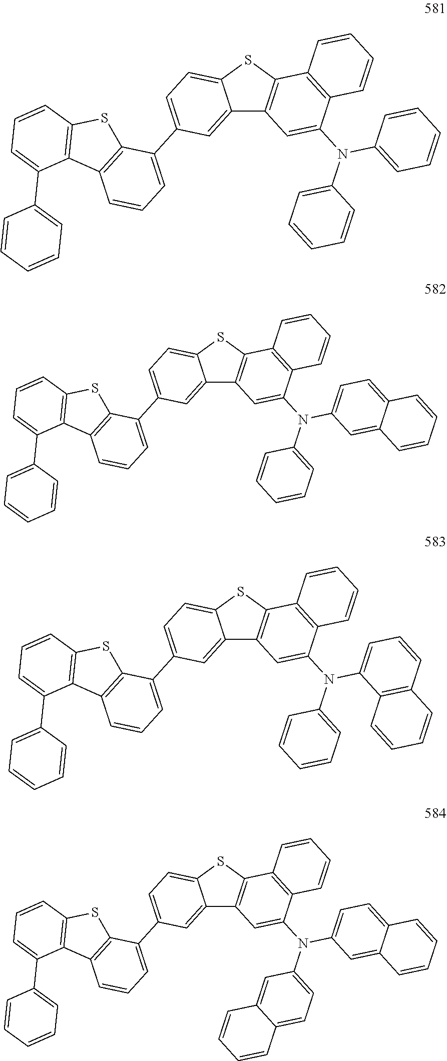 [SMILES: c1ccc(-c2cccc3sc4c(-c5ccc6sc7c8ccccc8c(N(c8ccccc8)c8ccccc8)cc7c6c5)cccc4c23)cc1 ; c1ccc(-c2cccc3sc4c(-c5ccc6sc7c8ccccc8c(N(c8ccccc8)c8ccc9ccccc9c8)cc7c6c5)cccc4c23)cc1 ; c1ccc(-c2cccc3sc4c(-c5ccc6sc7c8ccccc8c(N(c8ccccc8)c8cccc9ccccc89)cc7c6c5)cccc4c23)cc1 ; c1ccc(-c2cccc3sc4c(-c5ccc6sc7c8ccccc8c(N(c8ccc9ccccc9c8)c8ccc9ccccc9c8)cc7c6c5)cccc4c23)cc1]

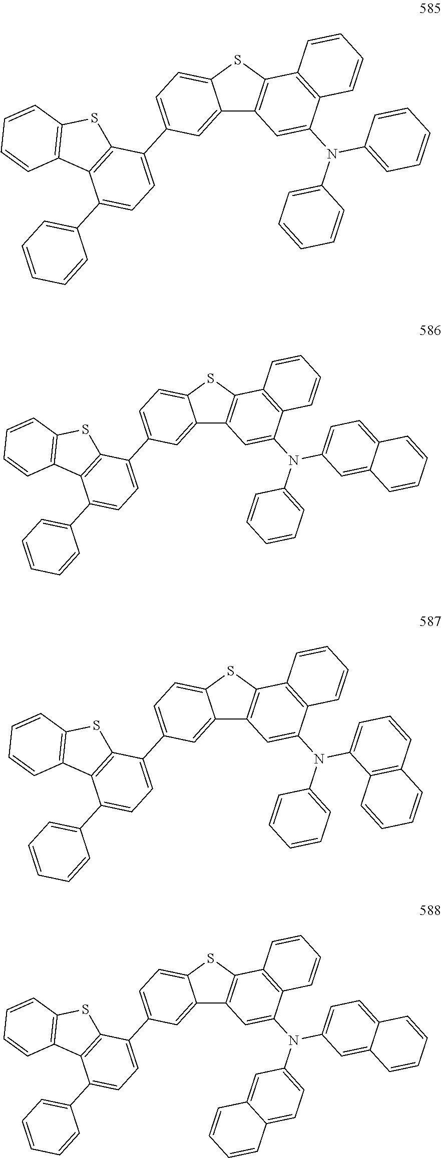 [SMILES: c1ccc(-c2ccc(-c3ccc4sc5c6ccccc6c(N(c6ccccc6)c6ccccc6)cc5c4c3)c3sc4ccccc4c23)cc1 ; c1ccc(-c2ccc(-c3ccc4sc5c6ccccc6c(N(c6ccccc6)c6ccc7ccccc7c6)cc5c4c3)c3sc4ccccc4c23)cc1 ; c1ccc(-c2ccc(-c3ccc4sc5c6ccccc6c(N(c6ccccc6)c6cccc7ccccc67)cc5c4c3)c3sc4ccccc4c23)cc1 ; c1ccc(-c2ccc(-c3ccc4sc5c6ccccc6c(N(c6ccc7ccccc7c6)c6ccc7ccccc7c6)cc5c4c3)c3sc4ccccc4c23)cc1]

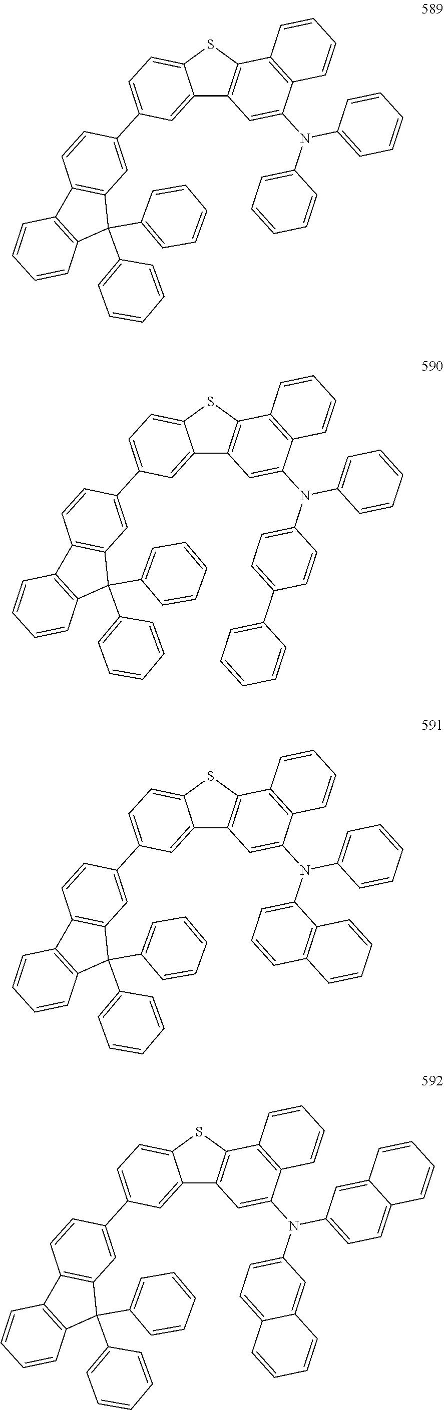 [SMILES: c1ccc(N(c2ccccc2)c2cc3c4cc(-c5ccc6c(c5)C(c5ccccc5)(c5ccccc5)c5ccccc5-6)ccc4sc3c3ccccc23)cc1 ; c1ccc(-c2ccc(N(c3ccccc3)c3cc4c5cc(-c6ccc7c(c6)C(c6ccccc6)(c6ccccc6)c6ccccc6-7)ccc5sc4c4ccccc34)cc2)cc1 ; c1ccc(N(c2cccc3ccccc23)c2cc3c4cc(-c5ccc6c(c5)C(c5ccccc5)(c5ccccc5)c5ccccc5-6)ccc4sc3c3ccccc23)cc1 ; c1ccc(C2(c3ccccc3)c3ccccc3-c3ccc(-c4ccc5sc6c7ccccc7c(N(c7ccc8ccccc8c7)c7ccc8ccccc8c7)cc6c5c4)cc32)cc1]

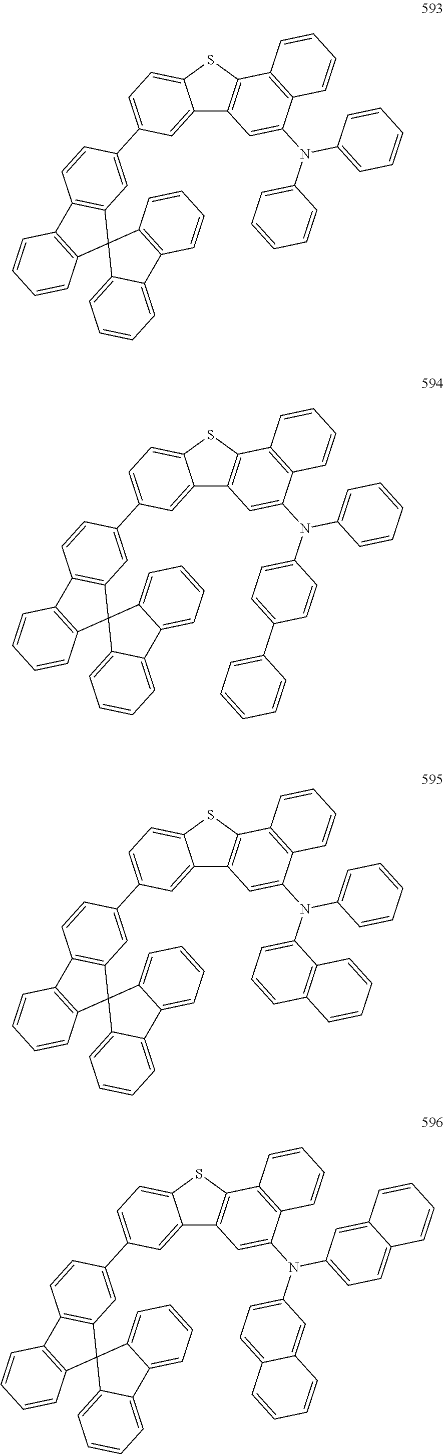 [SMILES: c1ccc(N(c2ccccc2)c2cc3c4cc(-c5ccc6c(c5)C5(c7ccccc7-c7ccccc75)c5ccccc5-6)ccc4sc3c3ccccc23)cc1 ; c1ccc(-c2ccc(N(c3ccccc3)c3cc4c5cc(-c6ccc7c(c6)C6(c8ccccc8-c8ccccc86)c6ccccc6-7)ccc5sc4c4ccccc34)cc2)cc1 ; c1ccc(N(c2cccc3ccccc23)c2cc3c4cc(-c5ccc6c(c5)C5(c7ccccc7-c7ccccc75)c5ccccc5-6)ccc4sc3c3ccccc23)cc1 ; c1ccc2c(c1)-c1ccccc1C21c2ccccc2-c2ccc(-c3ccc4sc5c6ccccc6c(N(c6ccc7ccccc7c6)c6ccc7ccccc7c6)cc5c4c3)cc21]

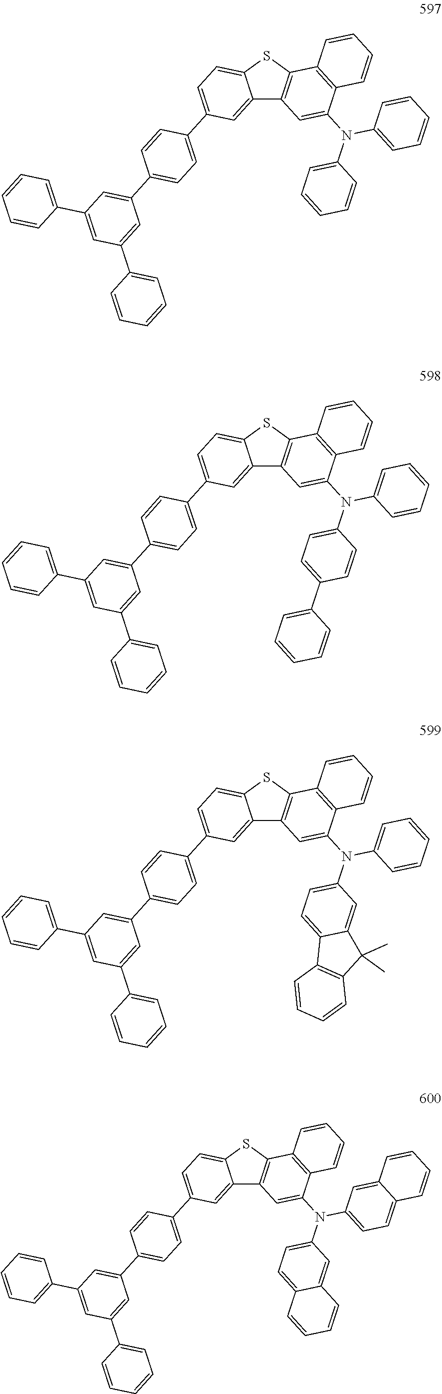 [SMILES: c1ccc(-c2cc(-c3ccccc3)cc(-c3ccc(-c4ccc5sc6c7ccccc7c(N(c7ccccc7)c7ccccc7)cc6c5c4)cc3)c2)cc1 ; c1ccc(-c2ccc(N(c3ccccc3)c3cc4c5cc(-c6ccc(-c7cc(-c8ccccc8)cc(-c8ccccc8)c7)cc6)ccc5sc4c4ccccc34)cc2)cc1 ; CC1(C)c2ccccc2-c2ccc(N(c3ccccc3)c3cc4c5cc(-c6ccc(-c7cc(-c8ccccc8)cc(-c8ccccc8)c7)cc6)ccc5sc4c4ccccc34)cc21 ; c1ccc(-c2cc(-c3ccccc3)cc(-c3ccc(-c4ccc5sc6c7ccccc7c(N(c7ccc8ccccc8c7)c7ccc8ccccc8c7)cc6c5c4)cc3)c2)cc1]

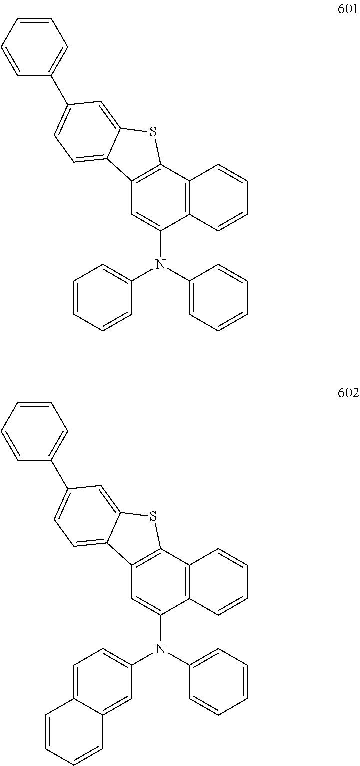 [SMILES: c1ccc(-c2ccc3c(c2)sc2c4ccccc4c(N(c4ccccc4)c4ccccc4)cc32)cc1 ; c1ccc(-c2ccc3c(c2)sc2c4ccccc4c(N(c4ccccc4)c4ccc5ccccc5c4)cc32)cc1]

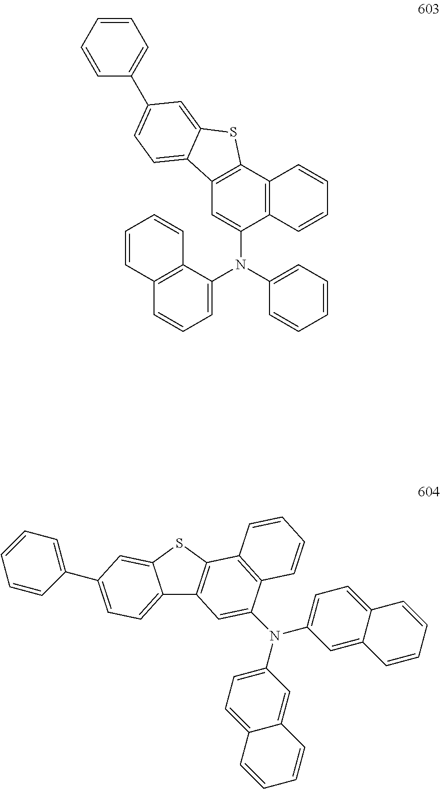 [SMILES: c1ccc(-c2ccc3c(c2)sc2c4ccccc4c(N(c4ccccc4)c4cccc5ccccc45)cc32)cc1 ; c1ccc(-c2ccc3c(c2)sc2c4ccccc4c(N(c4ccc5ccccc5c4)c4ccc5ccccc5c4)cc32)cc1]

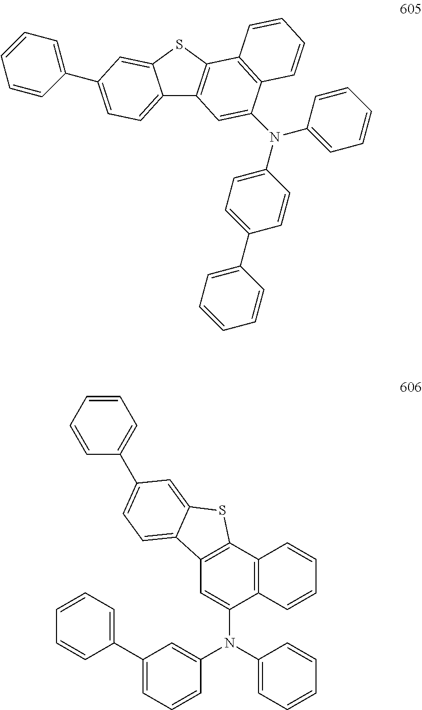 [SMILES: c1ccc(-c2ccc(N(c3ccccc3)c3cc4c5ccc(-c6ccccc6)cc5sc4c4ccccc34)cc2)cc1 ; c1ccc(-c2cccc(N(c3ccccc3)c3cc4c5ccc(-c6ccccc6)cc5sc4c4ccccc34)c2)cc1]

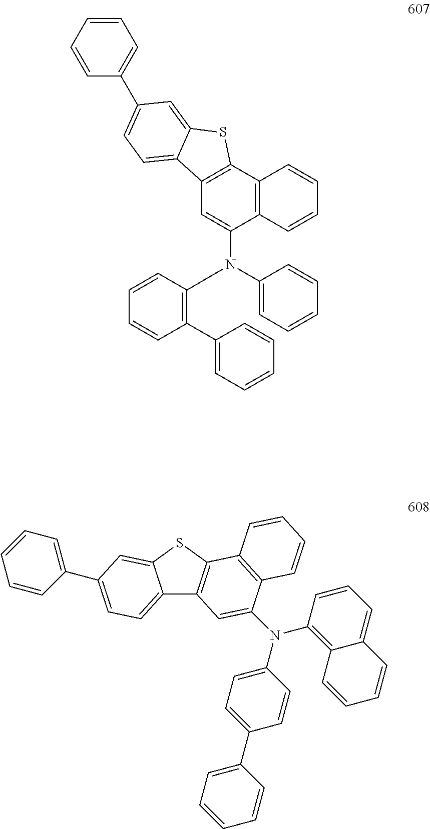 [SMILES: c1ccc(-c2ccc3c(c2)sc2c4ccccc4c(N(c4ccccc4)c4ccccc4-c4ccccc4)cc32)cc1 ; c1ccc(-c2ccc(N(c3cccc4ccccc34)c3cc4c5ccc(-c6ccccc6)cc5sc4c4ccccc34)cc2)cc1]

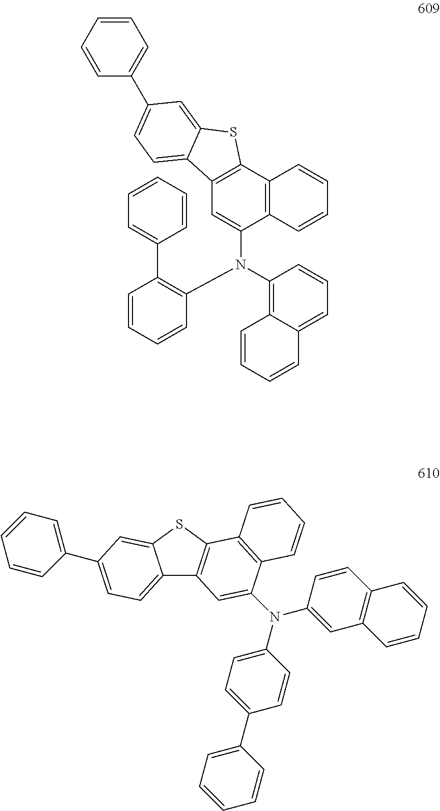 [SMILES: c1ccc(-c2ccc3c(c2)sc2c4ccccc4c(N(c4ccccc4-c4ccccc4)c4cccc5ccccc45)cc32)cc1 ; c1ccc(-c2ccc(N(c3ccc4ccccc4c3)c3cc4c5ccc(-c6ccccc6)cc5sc4c4ccccc34)cc2)cc1]

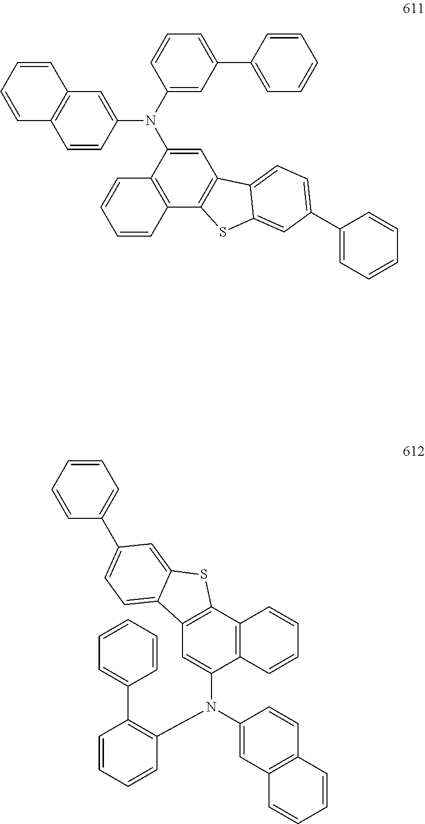 [SMILES: c1ccc(-c2cccc(N(c3ccc4ccccc4c3)c3cc4c5ccc(-c6ccccc6)cc5sc4c4ccccc34)c2)cc1 ; c1ccc(-c2ccc3c(c2)sc2c4ccccc4c(N(c4ccc5ccccc5c4)c4ccccc4-c4ccccc4)cc32)cc1]

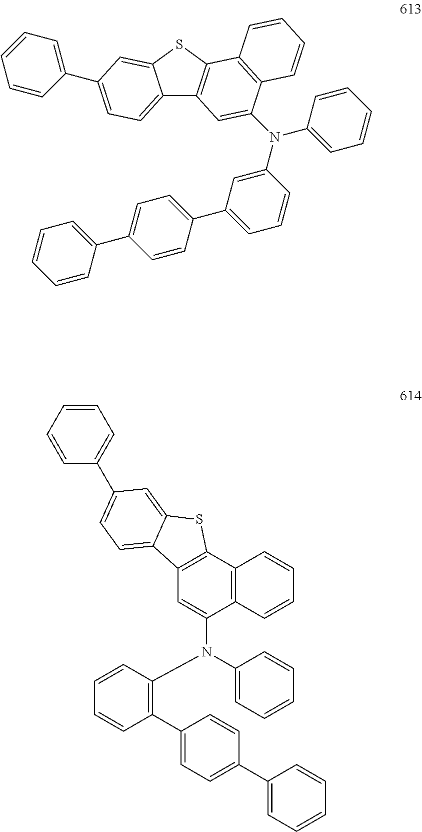 [SMILES: c1ccc(-c2ccc(-c3cccc(N(c4ccccc4)c4cc5c6ccc(-c7ccccc7)cc6sc5c5ccccc45)c3)cc2)cc1 ; c1ccc(-c2ccc(-c3ccccc3N(c3ccccc3)c3cc4c5ccc(-c6ccccc6)cc5sc4c4ccccc34)cc2)cc1]

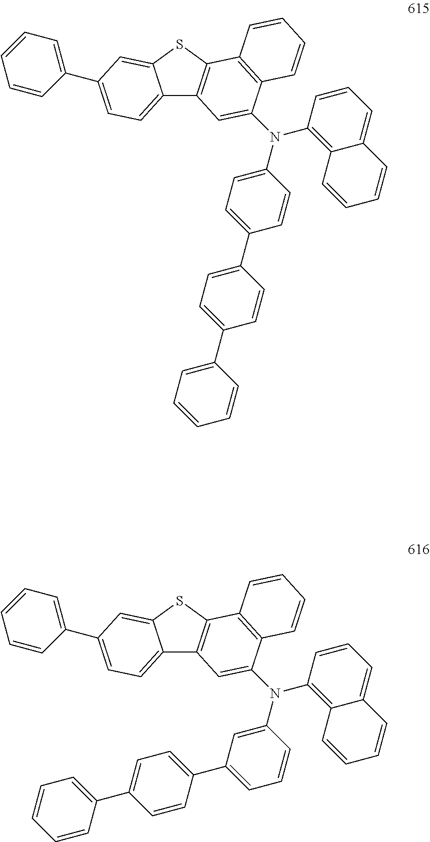 [SMILES: c1ccc(-c2ccc(-c3ccc(N(c4cccc5ccccc45)c4cc5c6ccc(-c7ccccc7)cc6sc5c5ccccc45)cc3)cc2)cc1 ; c1ccc(-c2ccc(-c3cccc(N(c4cccc5ccccc45)c4cc5c6ccc(-c7ccccc7)cc6sc5c5ccccc45)c3)cc2)cc1]

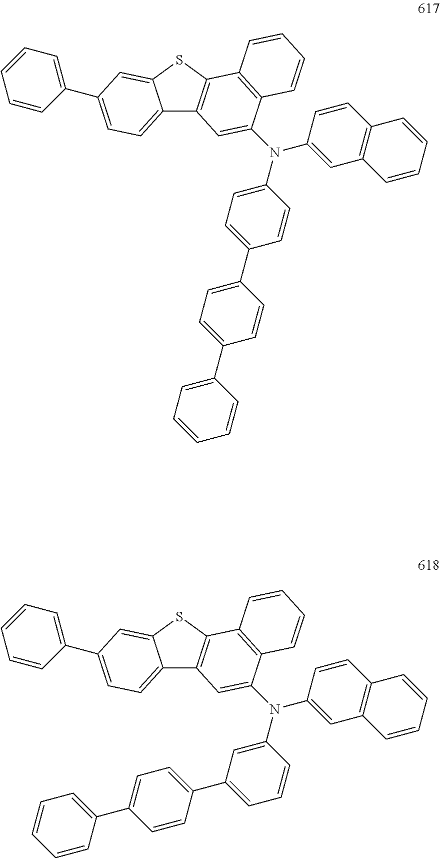 [SMILES: c1ccc(-c2ccc(-c3ccc(N(c4ccc5ccccc5c4)c4cc5c6ccc(-c7ccccc7)cc6sc5c5ccccc45)cc3)cc2)cc1 ; c1ccc(-c2ccc(-c3cccc(N(c4ccc5ccccc5c4)c4cc5c6ccc(-c7ccccc7)cc6sc5c5ccccc45)c3)cc2)cc1]

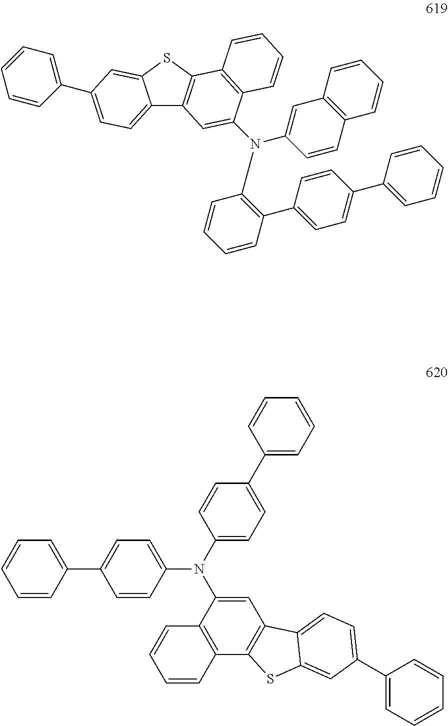 [SMILES: c1ccc(-c2ccc(-c3ccccc3N(c3ccc4ccccc4c3)c3cc4c5ccc(-c6ccccc6)cc5sc4c4ccccc34)cc2)cc1 ; c1ccc(-c2ccc(N(c3ccc(-c4ccccc4)cc3)c3cc4c5ccc(-c6ccccc6)cc5sc4c4ccccc34)cc2)cc1]

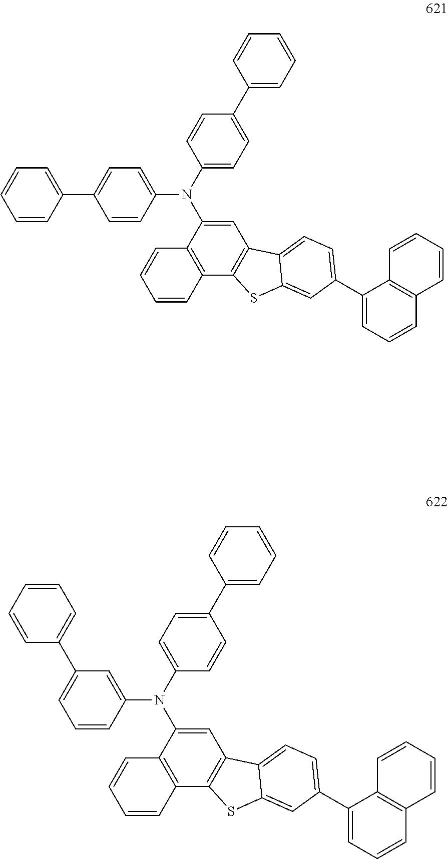 [SMILES: c1ccc(-c2ccc(N(c3ccc(-c4ccccc4)cc3)c3cc4c5ccc(-c6cccc7ccccc67)cc5sc4c4ccccc34)cc2)cc1 ; c1ccc(-c2ccc(N(c3cccc(-c4ccccc4)c3)c3cc4c5ccc(-c6cccc7ccccc67)cc5sc4c4ccccc34)cc2)cc1]

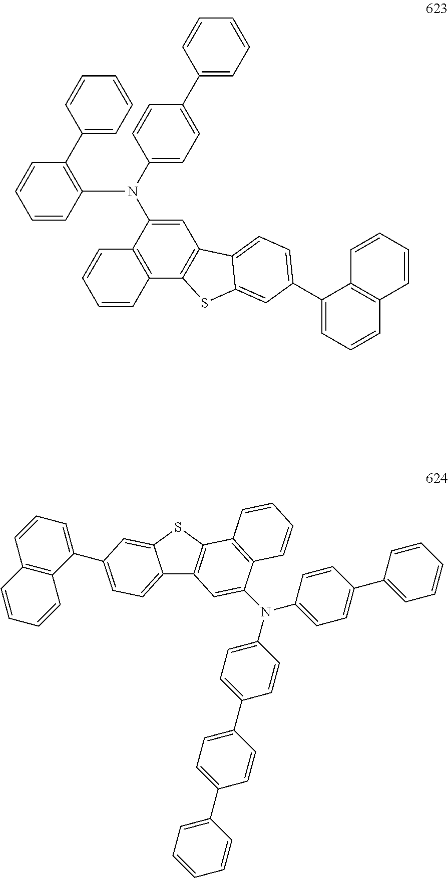 [SMILES: c1ccc(-c2ccc(N(c3ccccc3-c3ccccc3)c3cc4c5ccc(-c6cccc7ccccc67)cc5sc4c4ccccc34)cc2)cc1 ; c1ccc(-c2ccc(-c3ccc(N(c4ccc(-c5ccccc5)cc4)c4cc5c6ccc(-c7cccc8ccccc78)cc6sc5c5ccccc45)cc3)cc2)cc1]

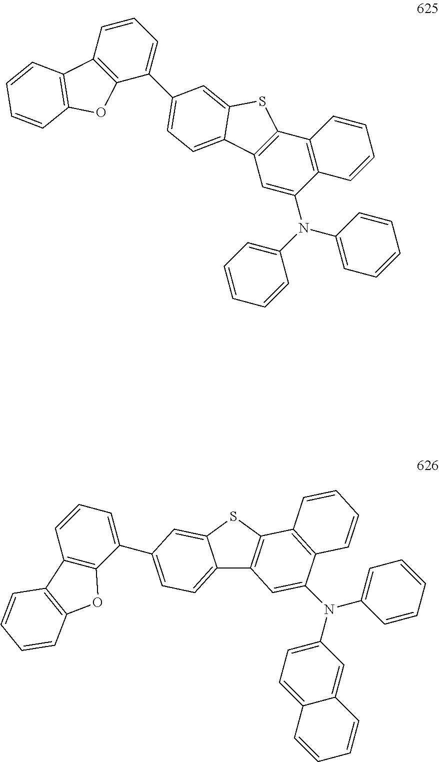 [SMILES: c1ccc(N(c2ccccc2)c2cc3c4ccc(-c5cccc6c5oc5ccccc56)cc4sc3c3ccccc23)cc1 ; c1ccc(N(c2ccc3ccccc3c2)c2cc3c4ccc(-c5cccc6c5oc5ccccc56)cc4sc3c3ccccc23)cc1]

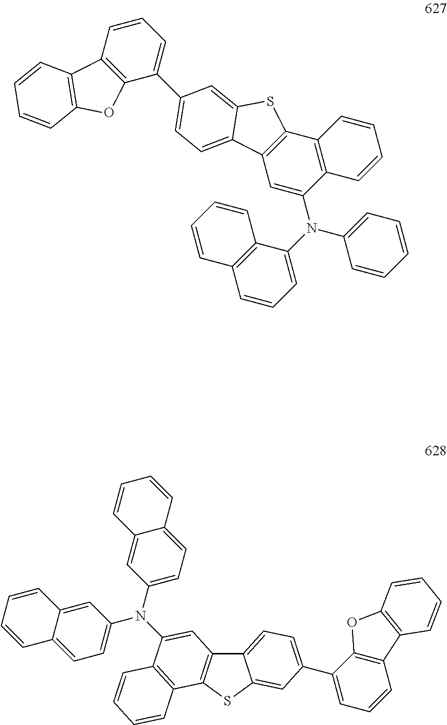 [SMILES: c1ccc(N(c2cccc3ccccc23)c2cc3c4ccc(-c5cccc6c5oc5ccccc56)cc4sc3c3ccccc23)cc1 ; c1ccc2cc(N(c3ccc4ccccc4c3)c3cc4c5ccc(-c6cccc7c6oc6ccccc67)cc5sc4c4ccccc34)ccc2c1]

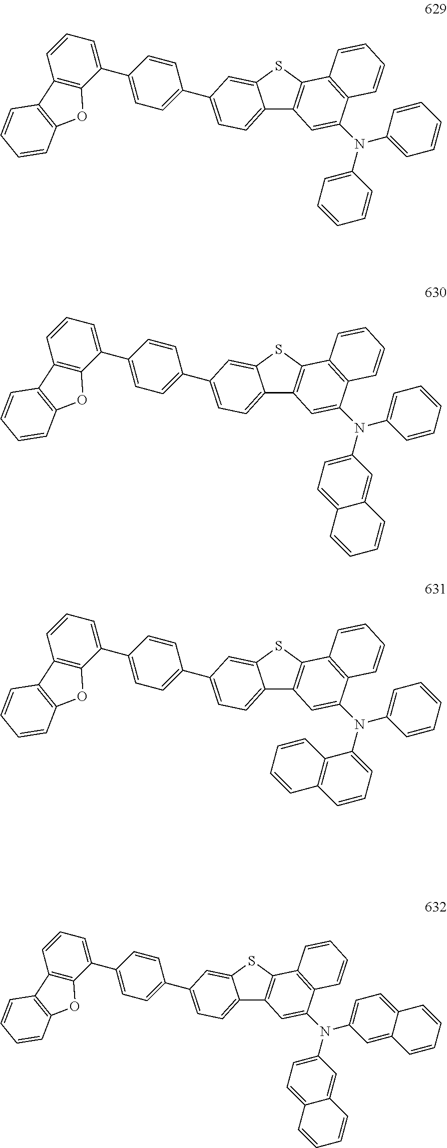 [SMILES: c1ccc(N(c2ccccc2)c2cc3c4ccc(-c5ccc(-c6cccc7c6oc6ccccc67)cc5)cc4sc3c3ccccc23)cc1 ; c1ccc(N(c2ccc3ccccc3c2)c2cc3c4ccc(-c5ccc(-c6cccc7c6oc6ccccc67)cc5)cc4sc3c3ccccc23)cc1 ; c1ccc(N(c2cccc3ccccc23)c2cc3c4ccc(-c5ccc(-c6cccc7c6oc6ccccc67)cc5)cc4sc3c3ccccc23)cc1 ; c1ccc2cc(N(c3ccc4ccccc4c3)c3cc4c5ccc(-c6ccc(-c7cccc8c7oc7ccccc78)cc6)cc5sc4c4ccccc34)ccc2c1]

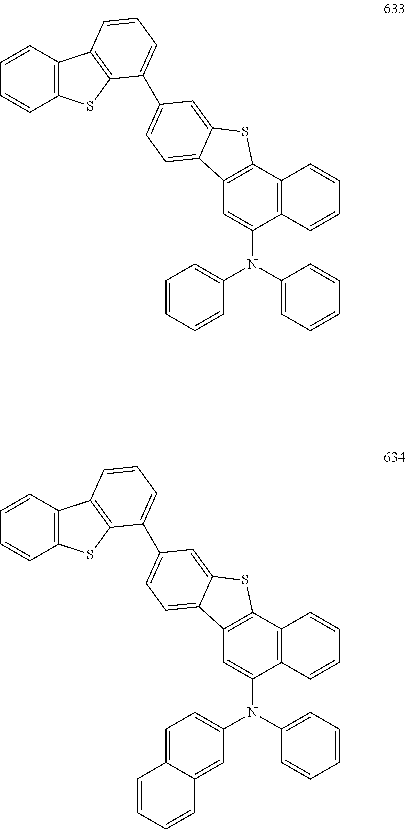 [SMILES: c1ccc(N(c2ccccc2)c2cc3c4ccc(-c5cccc6c5sc5ccccc56)cc4sc3c3ccccc23)cc1 ; c1ccc(N(c2ccc3ccccc3c2)c2cc3c4ccc(-c5cccc6c5sc5ccccc56)cc4sc3c3ccccc23)cc1]

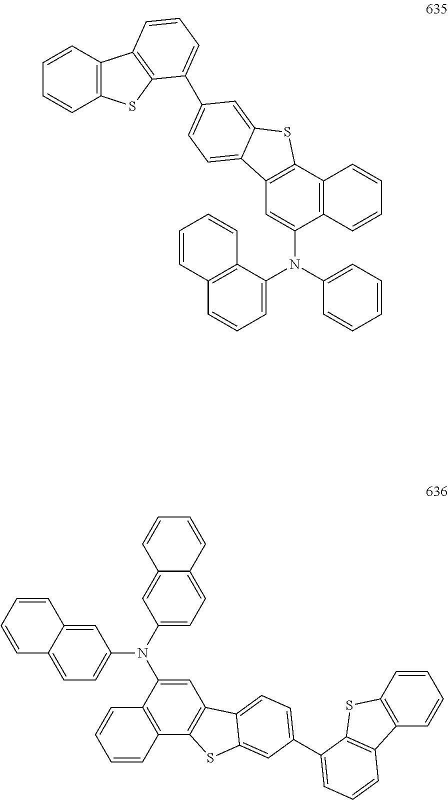 [SMILES: c1ccc(N(c2cccc3ccccc23)c2cc3c4ccc(-c5cccc6c5sc5ccccc56)cc4sc3c3ccccc23)cc1 ; c1ccc2cc(N(c3ccc4ccccc4c3)c3cc4c5ccc(-c6cccc7c6sc6ccccc67)cc5sc4c4ccccc34)ccc2c1]

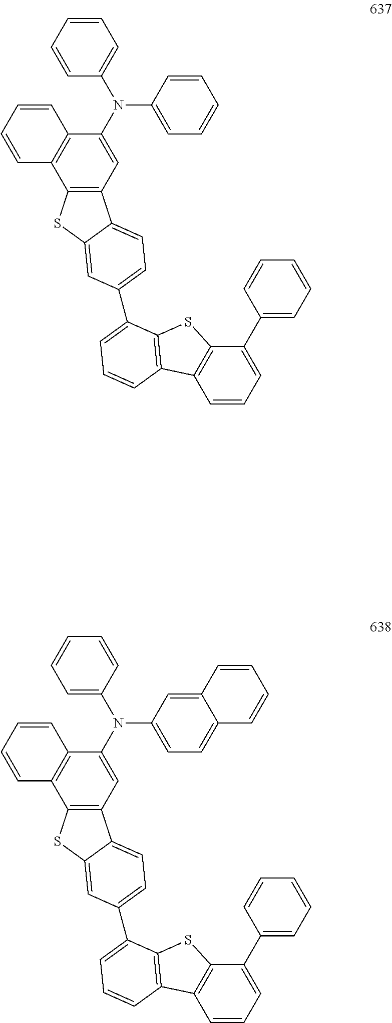 [SMILES: c1ccc(-c2cccc3c2sc2c(-c4ccc5c(c4)sc4c6ccccc6c(N(c6ccccc6)c6ccccc6)cc54)cccc23)cc1 ; c1ccc(-c2cccc3c2sc2c(-c4ccc5c(c4)sc4c6ccccc6c(N(c6ccccc6)c6ccc7ccccc7c6)cc54)cccc23)cc1]

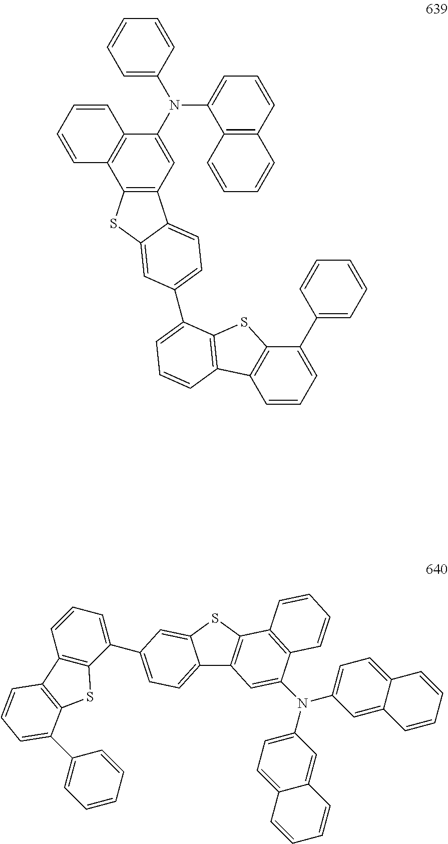 [SMILES: c1ccc(-c2cccc3c2sc2c(-c4ccc5c(c4)sc4c6ccccc6c(N(c6ccccc6)c6cccc7ccccc67)cc54)cccc23)cc1 ; c1ccc(-c2cccc3c2sc2c(-c4ccc5c(c4)sc4c6ccccc6c(N(c6ccc7ccccc7c6)c6ccc7ccccc7c6)cc54)cccc23)cc1]

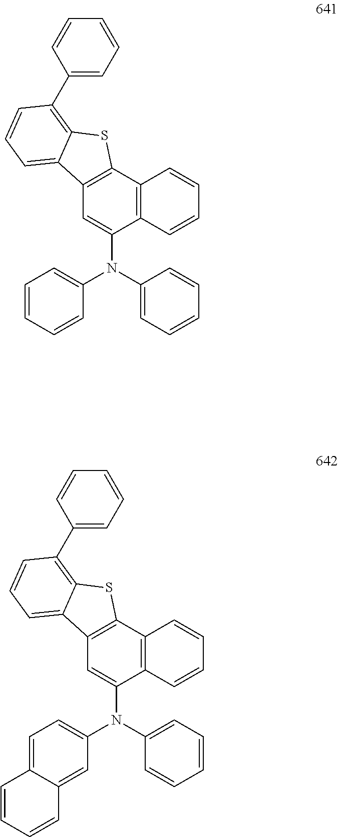 [SMILES: c1ccc(-c2cccc3c2sc2c4ccccc4c(N(c4ccccc4)c4ccccc4)cc32)cc1 ; c1ccc(-c2cccc3c2sc2c4ccccc4c(N(c4ccccc4)c4ccc5ccccc5c4)cc32)cc1]

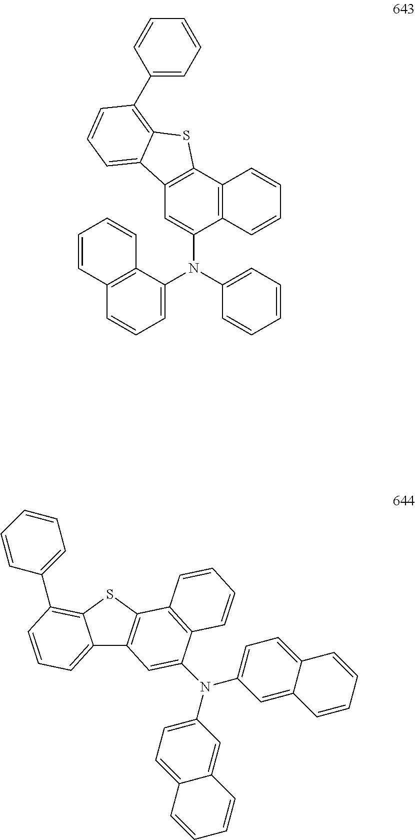 [SMILES: c1ccc(-c2cccc3c2sc2c4ccccc4c(N(c4ccccc4)c4cccc5ccccc45)cc32)cc1 ; c1ccc(-c2cccc3c2sc2c4ccccc4c(N(c4ccc5ccccc5c4)c4ccc5ccccc5c4)cc32)cc1]

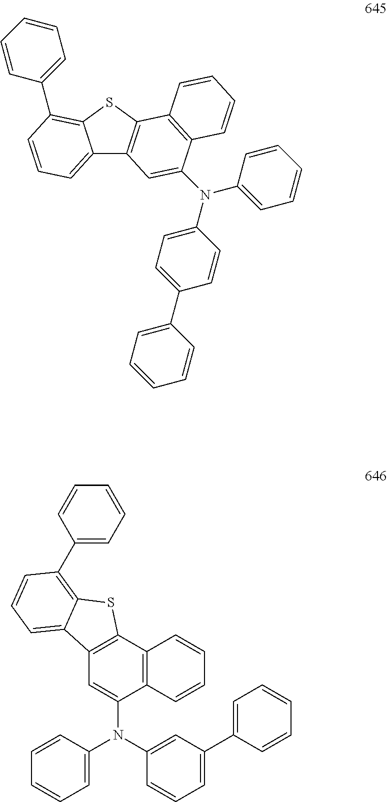 [SMILES: c1ccc(-c2ccc(N(c3ccccc3)c3cc4c5cccc(-c6ccccc6)c5sc4c4ccccc34)cc2)cc1 ; c1ccc(-c2cccc(N(c3ccccc3)c3cc4c5cccc(-c6ccccc6)c5sc4c4ccccc34)c2)cc1]

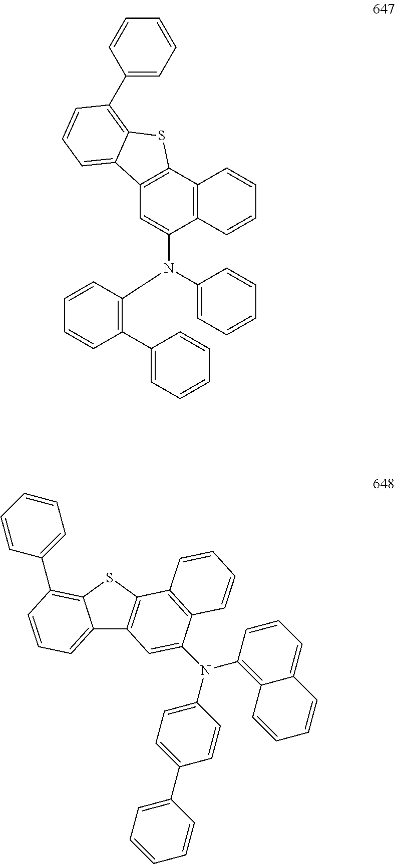 [SMILES: c1ccc(-c2ccccc2N(c2ccccc2)c2cc3c4cccc(-c5ccccc5)c4sc3c3ccccc23)cc1 ; c1ccc(-c2ccc(N(c3cccc4ccccc34)c3cc4c5cccc(-c6ccccc6)c5sc4c4ccccc34)cc2)cc1]

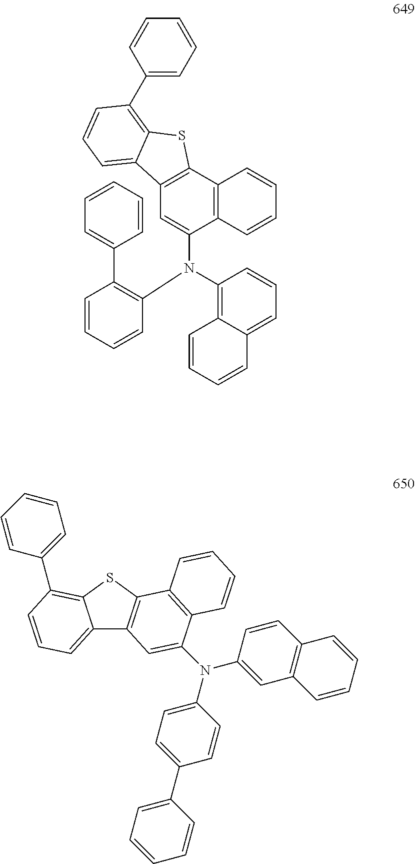 [SMILES: c1ccc(-c2ccccc2N(c2cccc3ccccc23)c2cc3c4cccc(-c5ccccc5)c4sc3c3ccccc23)cc1 ; c1ccc(-c2ccc(N(c3ccc4ccccc4c3)c3cc4c5cccc(-c6ccccc6)c5sc4c4ccccc34)cc2)cc1]

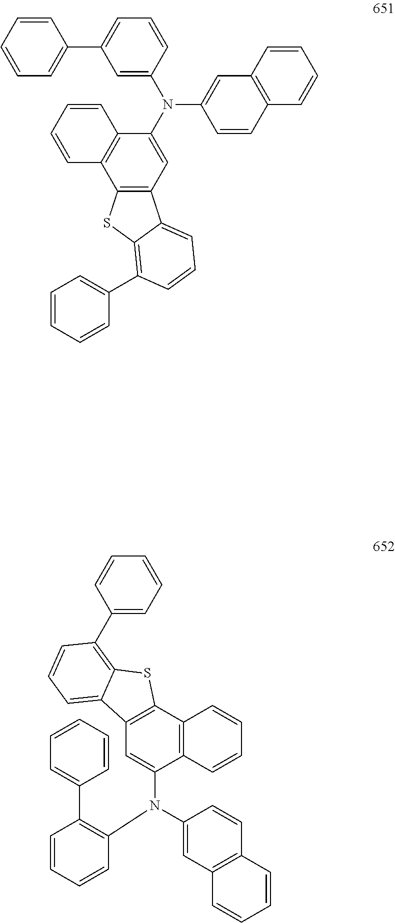 [SMILES: c1ccc(-c2cccc(N(c3ccc4ccccc4c3)c3cc4c5cccc(-c6ccccc6)c5sc4c4ccccc34)c2)cc1 ; c1ccc(-c2ccccc2N(c2ccc3ccccc3c2)c2cc3c4cccc(-c5ccccc5)c4sc3c3ccccc23)cc1]

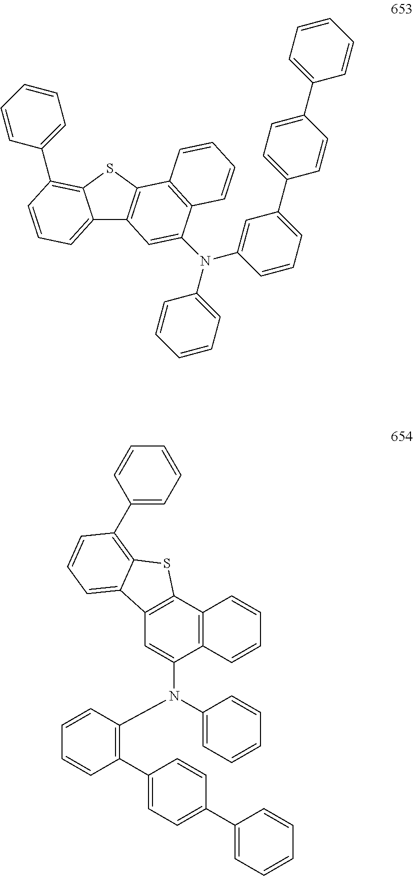 [SMILES: c1ccc(-c2ccc(-c3cccc(N(c4ccccc4)c4cc5c6cccc(-c7ccccc7)c6sc5c5ccccc45)c3)cc2)cc1 ; c1ccc(-c2ccc(-c3ccccc3N(c3ccccc3)c3cc4c5cccc(-c6ccccc6)c5sc4c4ccccc34)cc2)cc1]

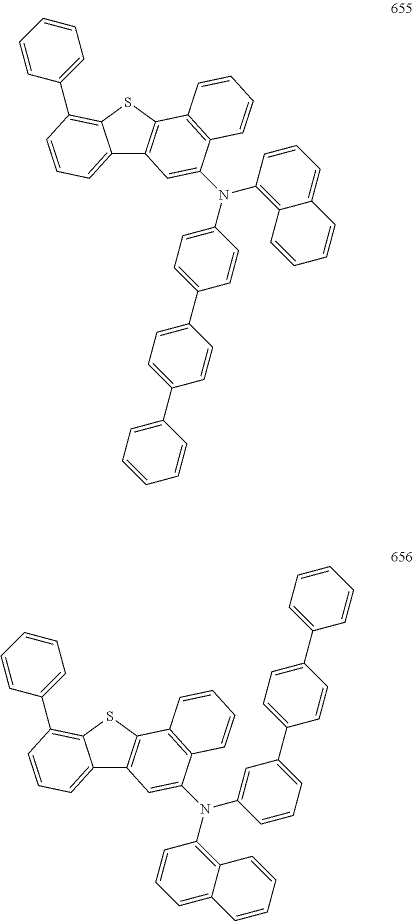 [SMILES: c1ccc(-c2ccc(-c3ccc(N(c4cccc5ccccc45)c4cc5c6cccc(-c7ccccc7)c6sc5c5ccccc45)cc3)cc2)cc1 ; c1ccc(-c2ccc(-c3cccc(N(c4cccc5ccccc45)c4cc5c6cccc(-c7ccccc7)c6sc5c5ccccc45)c3)cc2)cc1]

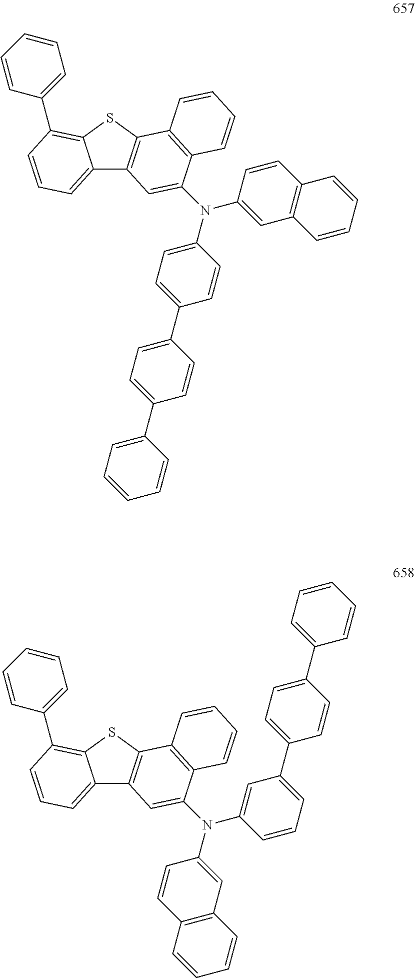 [SMILES: c1ccc(-c2ccc(-c3ccc(N(c4ccc5ccccc5c4)c4cc5c6cccc(-c7ccccc7)c6sc5c5ccccc45)cc3)cc2)cc1 ; c1ccc(-c2ccc(-c3cccc(N(c4ccc5ccccc5c4)c4cc5c6cccc(-c7ccccc7)c6sc5c5ccccc45)c3)cc2)cc1]

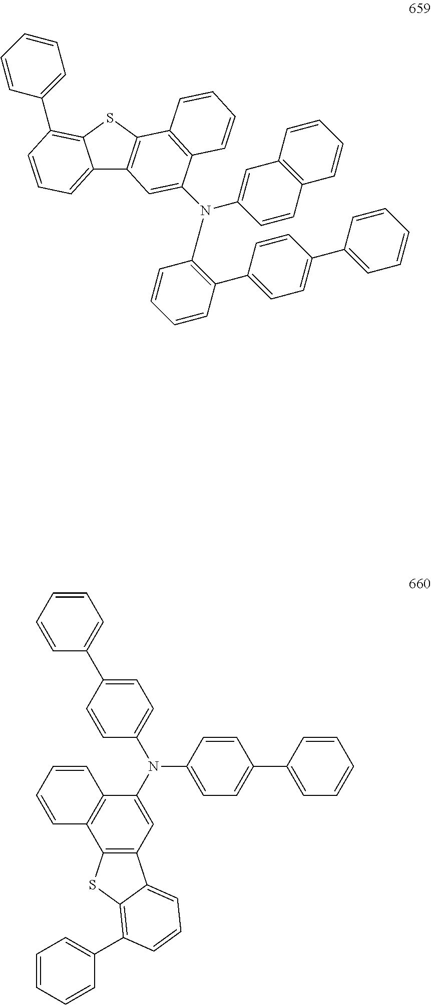 [SMILES: c1ccc(-c2ccc(-c3ccccc3N(c3ccc4ccccc4c3)c3cc4c5cccc(-c6ccccc6)c5sc4c4ccccc34)cc2)cc1 ; c1ccc(-c2ccc(N(c3ccc(-c4ccccc4)cc3)c3cc4c5cccc(-c6ccccc6)c5sc4c4ccccc34)cc2)cc1]

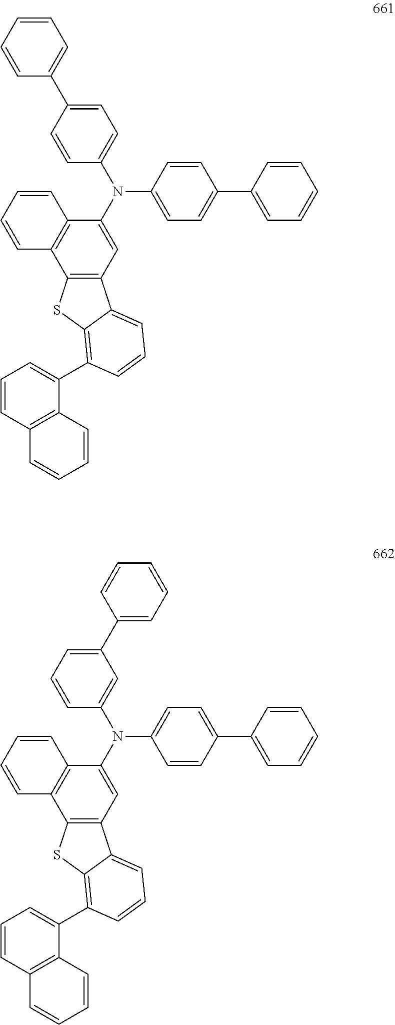 [SMILES: c1ccc(-c2ccc(N(c3ccc(-c4ccccc4)cc3)c3cc4c5cccc(-c6cccc7ccccc67)c5sc4c4ccccc34)cc2)cc1 ; c1ccc(-c2ccc(N(c3cccc(-c4ccccc4)c3)c3cc4c5cccc(-c6cccc7ccccc67)c5sc4c4ccccc34)cc2)cc1]

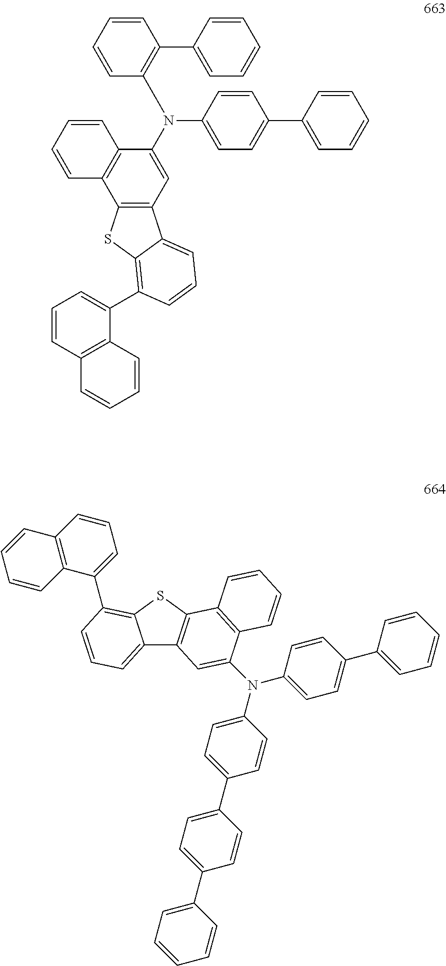 [SMILES: c1ccc(-c2ccc(N(c3ccccc3-c3ccccc3)c3cc4c5cccc(-c6cccc7ccccc67)c5sc4c4ccccc34)cc2)cc1 ; c1ccc(-c2ccc(-c3ccc(N(c4ccc(-c5ccccc5)cc4)c4cc5c6cccc(-c7cccc8ccccc78)c6sc5c5ccccc45)cc3)cc2)cc1]

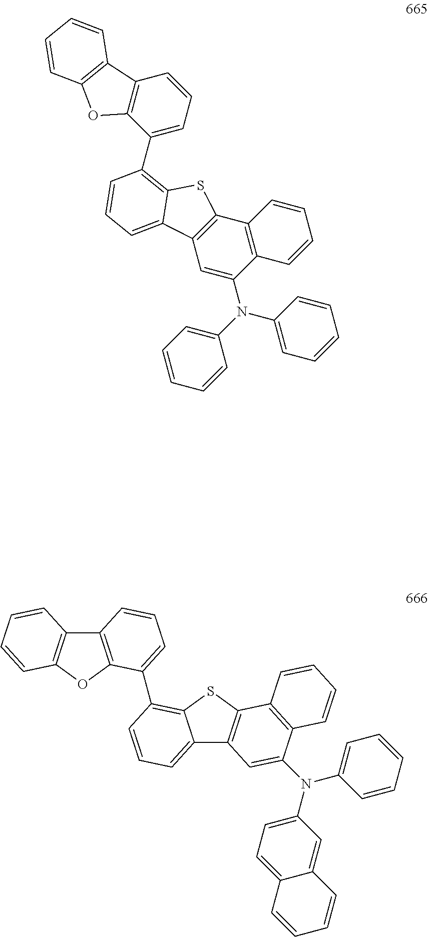 [SMILES: c1ccc(N(c2ccccc2)c2cc3c4cccc(-c5cccc6c5oc5ccccc56)c4sc3c3ccccc23)cc1 ; c1ccc(N(c2ccc3ccccc3c2)c2cc3c4cccc(-c5cccc6c5oc5ccccc56)c4sc3c3ccccc23)cc1]

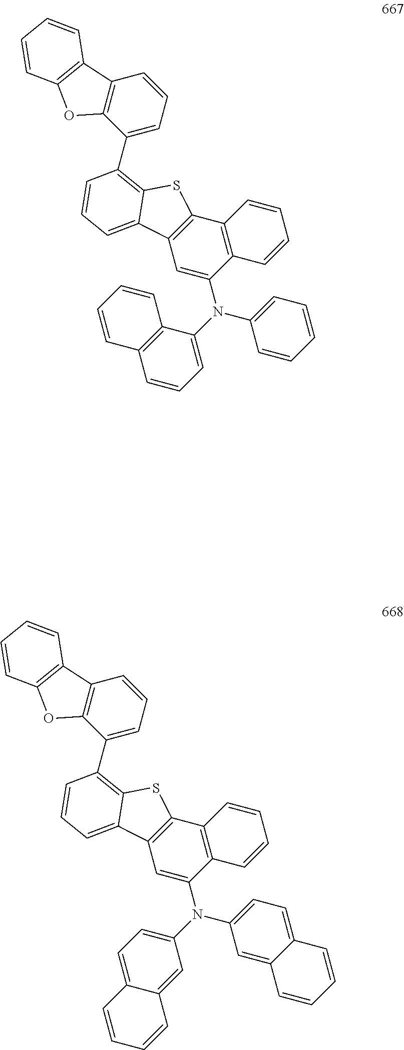 [SMILES: c1ccc(N(c2cccc3ccccc23)c2cc3c4cccc(-c5cccc6c5oc5ccccc56)c4sc3c3ccccc23)cc1 ; c1ccc2cc(N(c3ccc4ccccc4c3)c3cc4c5cccc(-c6cccc7c6oc6ccccc67)c5sc4c4ccccc34)ccc2c1]

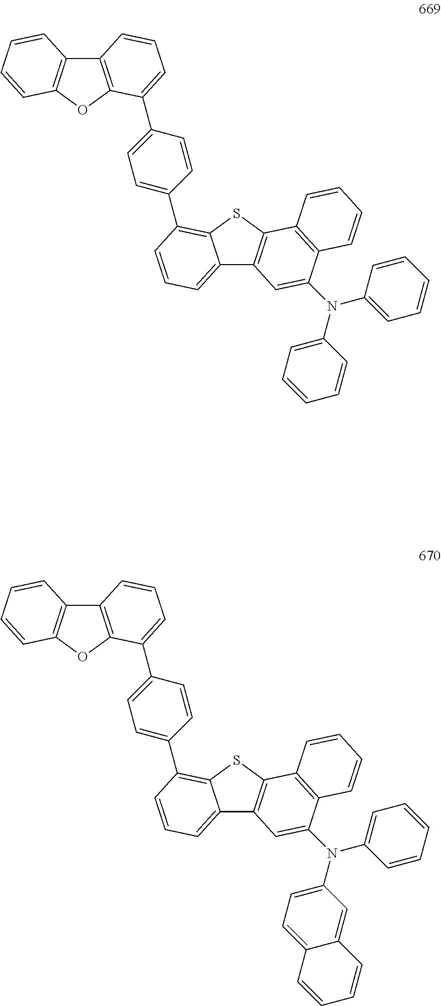 [SMILES: c1ccc(N(c2ccccc2)c2cc3c4cccc(-c5ccc(-c6cccc7c6oc6ccccc67)cc5)c4sc3c3ccccc23)cc1 ; c1ccc(N(c2ccc3ccccc3c2)c2cc3c4cccc(-c5ccc(-c6cccc7c6oc6ccccc67)cc5)c4sc3c3ccccc23)cc1]

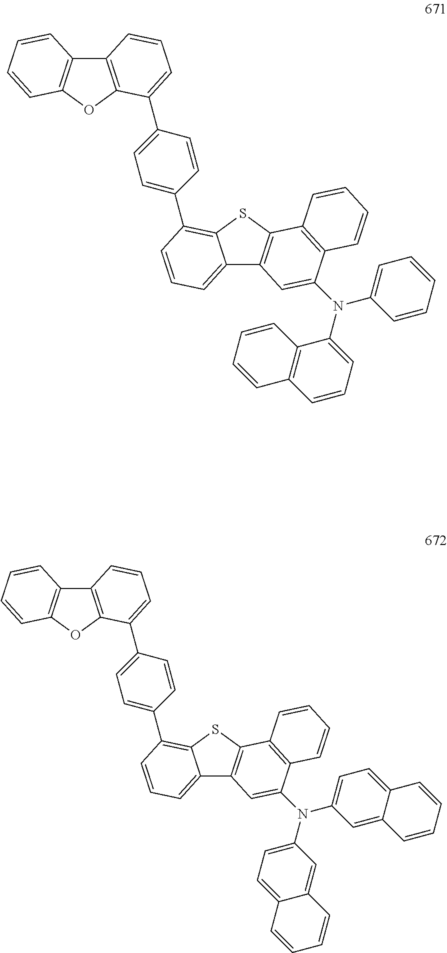 [SMILES: c1ccc(N(c2cccc3ccccc23)c2cc3c4cccc(-c5ccc(-c6cccc7c6oc6ccccc67)cc5)c4sc3c3ccccc23)cc1 ; c1ccc2cc(N(c3ccc4ccccc4c3)c3cc4c5cccc(-c6ccc(-c7cccc8c7oc7ccccc78)cc6)c5sc4c4ccccc34)ccc2c1]

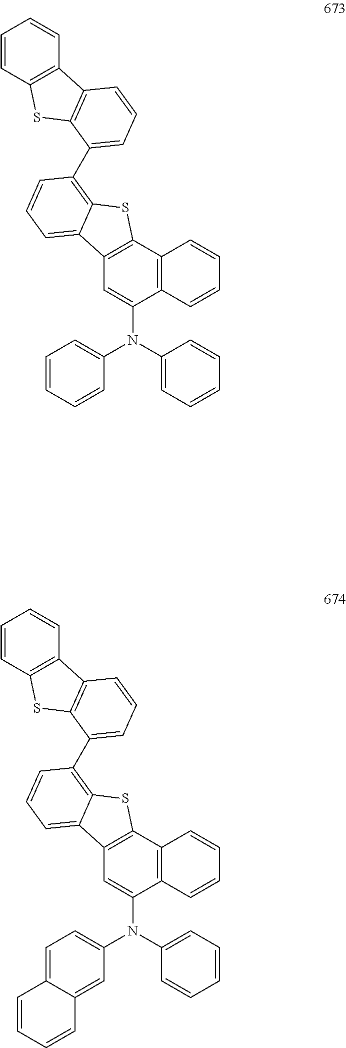 [SMILES: c1ccc(N(c2ccccc2)c2cc3c4cccc(-c5cccc6c5sc5ccccc56)c4sc3c3ccccc23)cc1 ; c1ccc(N(c2ccc3ccccc3c2)c2cc3c4cccc(-c5cccc6c5sc5ccccc56)c4sc3c3ccccc23)cc1]

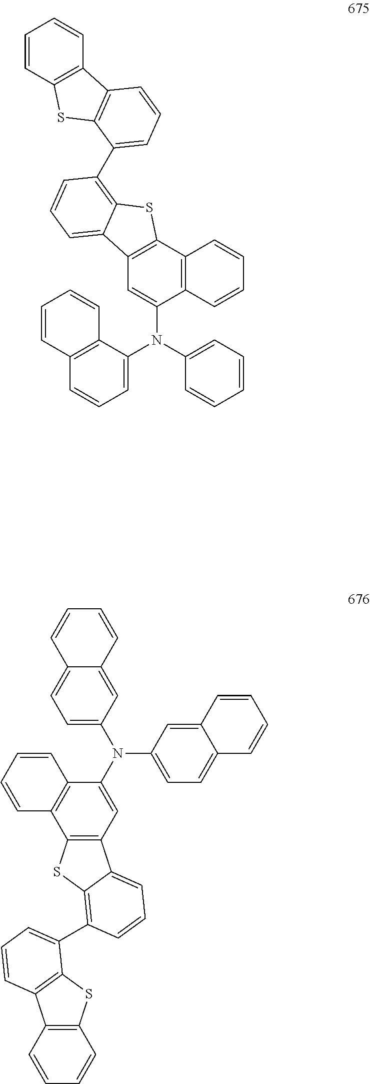 [SMILES: c1ccc(N(c2cccc3ccccc23)c2cc3c4cccc(-c5cccc6c5sc5ccccc56)c4sc3c3ccccc23)cc1 ; c1ccc2cc(N(c3ccc4ccccc4c3)c3cc4c5cccc(-c6cccc7c6sc6ccccc67)c5sc4c4ccccc34)ccc2c1]

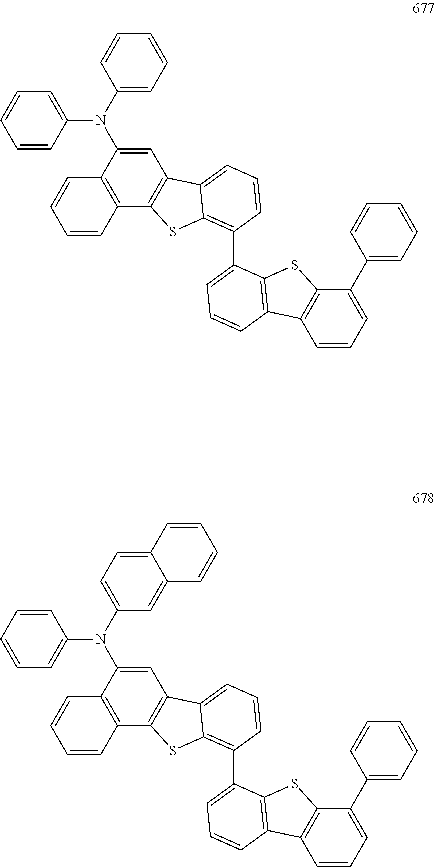 [SMILES: c1ccc(-c2cccc3c2sc2c(-c4cccc5c4sc4c6ccccc6c(N(c6ccccc6)c6ccccc6)cc54)cccc23)cc1 ; c1ccc(-c2cccc3c2sc2c(-c4cccc5c4sc4c6ccccc6c(N(c6ccccc6)c6ccc7ccccc7c6)cc54)cccc23)cc1]

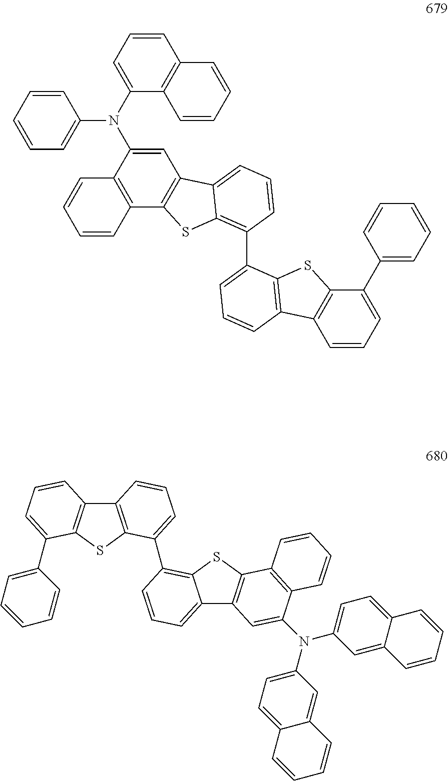 [SMILES: c1ccc(-c2cccc3c2sc2c(-c4cccc5c4sc4c6ccccc6c(N(c6ccccc6)c6cccc7ccccc67)cc54)cccc23)cc1 ; c1ccc(-c2cccc3c2sc2c(-c4cccc5c4sc4c6ccccc6c(N(c6ccc7ccccc7c6)c6ccc7ccccc7c6)cc54)cccc23)cc1]

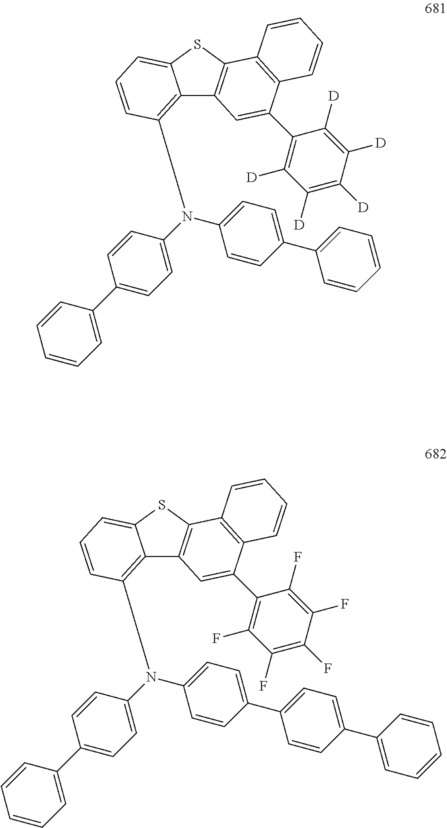 [SMILES: [2H]c1c([2H])c([2H])c(-c2cc3c(sc4cccc(N(c5ccc(-c6ccccc6)cc5)c5ccc(-c6ccccc6)cc5)c43)c3ccccc23)c([2H])c1[2H] ; Fc1c(F)c(F)c(-c2cc3c(sc4cccc(N(c5ccc(-c6ccccc6)cc5)c5ccc(-c6ccc(-c7ccccc7)cc6)cc5)c43)c3ccccc23)c(F)c1F]

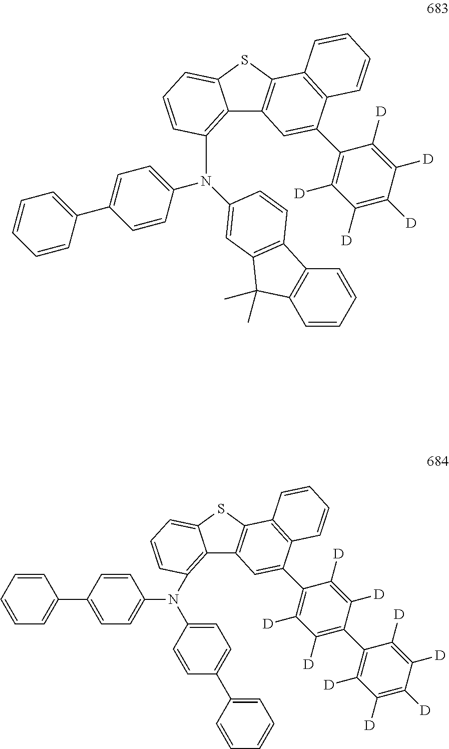 [SMILES: [2H]c1c([2H])c([2H])c(-c2cc3c(sc4cccc(N(c5ccc(-c6ccccc6)cc5)c5ccc6c(c5)C(C)(C)c5ccccc5-6)c43)c3ccccc23)c([2H])c1[2H] ; [2H]c1c([2H])c([2H])c(-c2c([2H])c([2H])c(-c3cc4c(sc5cccc(N(c6ccc(-c7ccccc7)cc6)c6ccc(-c7ccccc7)cc6)c54)c4ccccc34)c([2H])c2[2H])c([2H])c1[2H]]

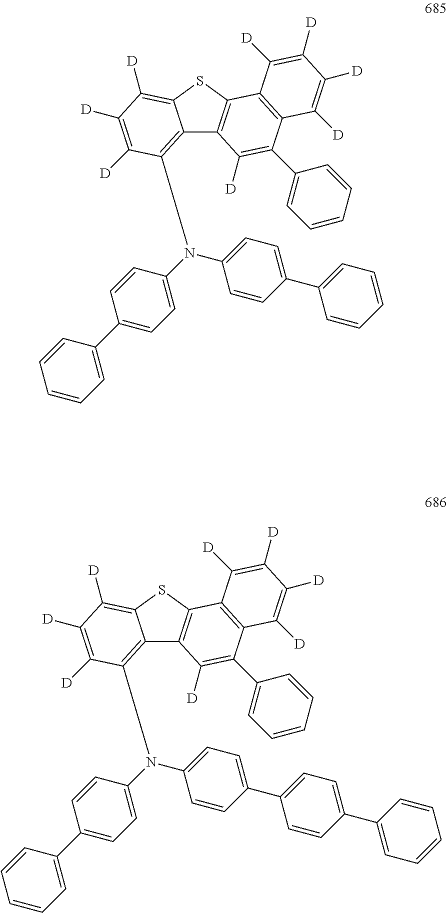 [SMILES: [2H]c1c([2H])c(N(c2ccc(-c3ccccc3)cc2)c2ccc(-c3ccccc3)cc2)c2c(sc3c4c([2H])c([2H])c([2H])c([2H])c4c(-c4ccccc4)c([2H])c32)c1[2H] ; [2H]c1c([2H])c(N(c2ccc(-c3ccccc3)cc2)c2ccc(-c3ccc(-c4ccccc4)cc3)cc2)c2c(sc3c4c([2H])c([2H])c([2H])c([2H])c4c(-c4ccccc4)c([2H])c32)c1[2H]]

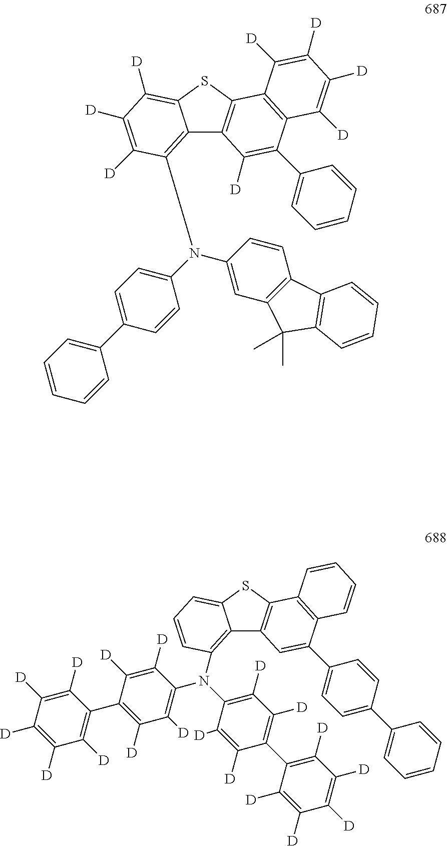 [SMILES: [2H]c1c([2H])c(N(c2ccc(-c3ccccc3)cc2)c2ccc3c(c2)C(C)(C)c2ccccc2-3)c2c(sc3c4c([2H])c([2H])c([2H])c([2H])c4c(-c4ccccc4)c([2H])c32)c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cccc4sc5c6ccccc6c(-c6ccc(-c7ccccc7)cc6)cc5c34)c([2H])c2[2H])c([2H])c1[2H]]

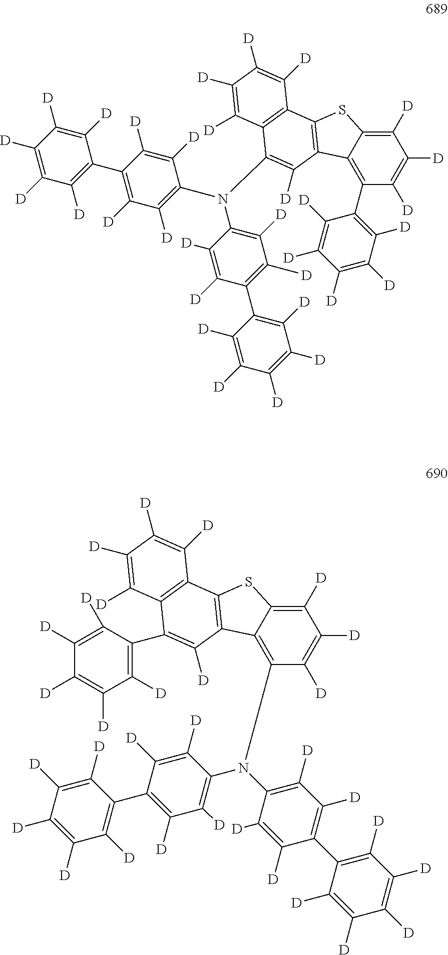

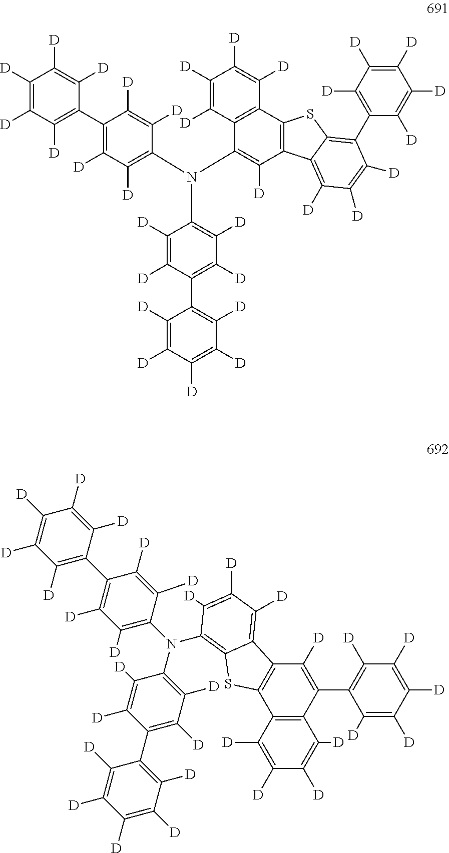

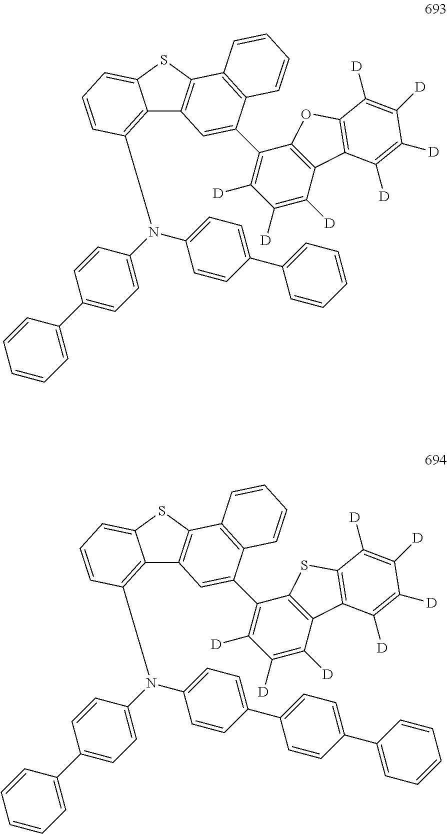 [SMILES: [2H]c1c([2H])c([2H])c2c(oc3c(-c4cc5c(sc6cccc(N(c7ccc(-c8ccccc8)cc7)c7ccc(-c8ccccc8)cc7)c65)c5ccccc45)c([2H])c([2H])c([2H])c32)c1[2H] ; [2H]c1c([2H])c([2H])c2c(sc3c(-c4cc5c(sc6cccc(N(c7ccc(-c8ccccc8)cc7)c7ccc(-c8ccc(-c9ccccc9)cc8)cc7)c65)c5ccccc45)c([2H])c([2H])c([2H])c32)c1[2H]]

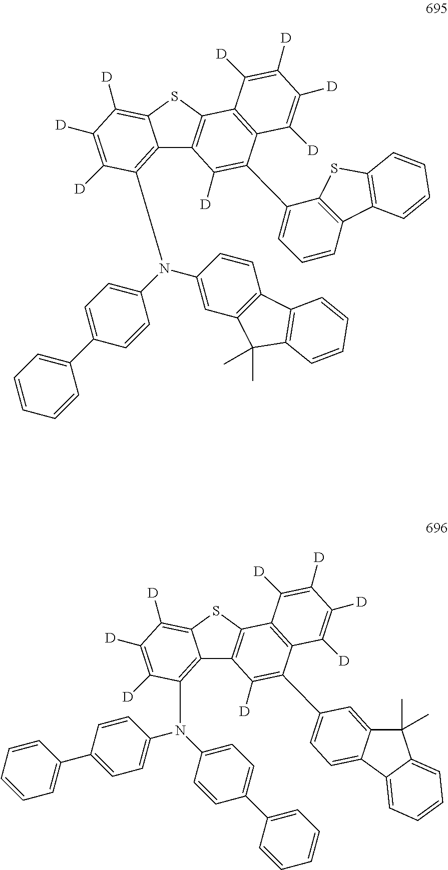 [SMILES: [2H]c1c([2H])c(N(c2ccc(-c3ccccc3)cc2)c2ccc3c(c2)C(C)(C)c2ccccc2-3)c2c(sc3c4c([2H])c([2H])c([2H])c([2H])c4c(-c4cccc5c4sc4ccccc45)c([2H])c32)c1[2H] ; [2H]c1c([2H])c(N(c2ccc(-c3ccccc3)cc2)c2ccc(-c3ccccc3)cc2)c2c(sc3c4c([2H])c([2H])c([2H])c([2H])c4c(-c4ccc5c(c4)C(C)(C)c4ccccc4-5)c([2H])c32)c1[2H]]

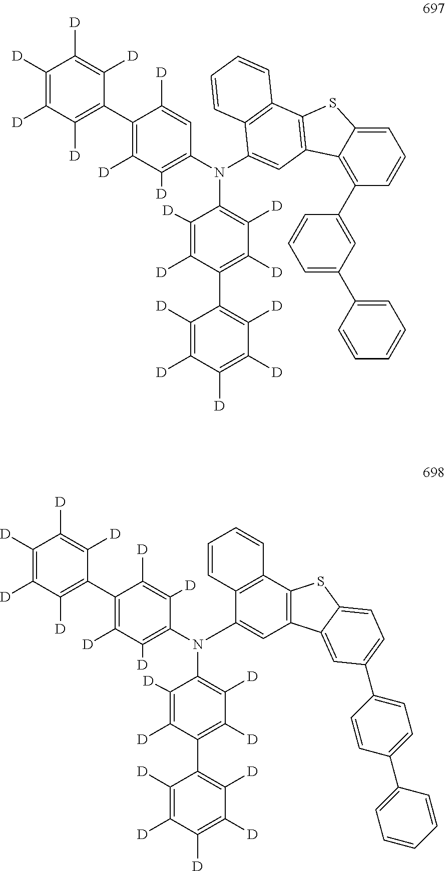 [SMILES: [2H]c1cc(N(c2c([2H])c([2H])c(-c3c([2H])c([2H])c([2H])c([2H])c3[2H])c([2H])c2[2H])c2cc3c(sc4cccc(-c5cccc(-c6ccccc6)c5)c43)c3ccccc23)c([2H])c([2H])c1-c1c([2H])c([2H])c([2H])c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4c5cc(-c6ccc(-c7ccccc7)cc6)ccc5sc4c4ccccc34)c([2H])c2[2H])c([2H])c1[2H]]

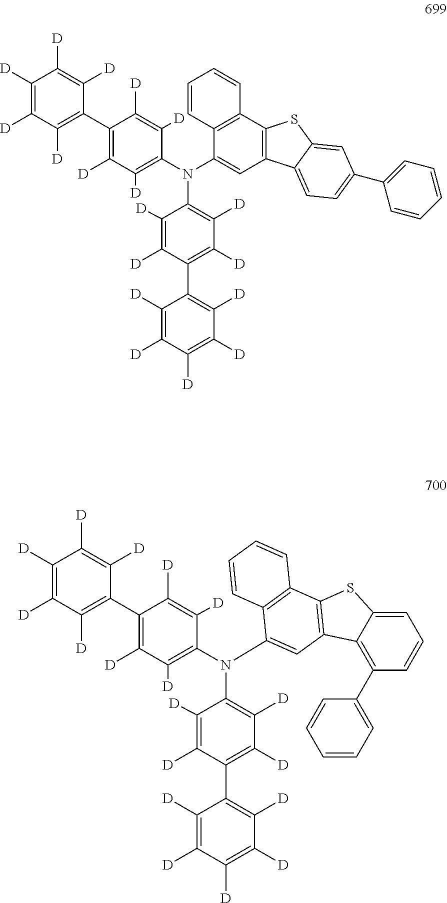 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4c5ccc(-c6ccccc6)cc5sc4c4ccccc34)c([2H])c2[2H])c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4c(sc5cccc(-c6ccccc6)c54)c4ccccc34)c([2H])c2[2H])c([2H])c1[2H]]

In addition, by introducing various substituents to the structure of Chemical Formula 1, compounds having unique properties of the introduced substituents may be synthesized. For example, by introducing substituents normally used as a hole injection layer material, an electron blocking layer material, a hole transport layer material, a light emitting layer material, an electron transport layer material, a hole blocking layer material and a charge generation layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may became diverse.

In addition, one embodiment of the present disclosure relates to an organic light emitting device comprising:
a first electrode;
a second electrode provided opposite to the first electrode; and
one or more organic material layers provided between the first electrode and the second electrode,
wherein at least one of the one or more layers of the organic material layers comprise the heterocyclic compound represented by Chemical Formula 1.

In one embodiment of the present disclosure, the first electrode may be a positive electrode, and the second electrode may be a negative electrode.

In another embodiment, the first electrode may be a negative electrode, and the second electrode may be a positive electrode.

In one embodiment of the present disclosure, the organic material layer may comprise one or more types selected from the group consisting of an electron injection layer, an electron transport layer, a hole blocking layer, a light emitting layer, a light emitting auxiliary layer, an electron blocking layer, a hole transport layer and a hole injection layer, and the one or more types of layers selected from the group consisting of an electron injection layer, an electron transport layer, a hole blocking layer, a light emitting layer, a light emitting auxiliary layer, an electron blocking layer, a hole transport layer and a hole injection layer may comprise the heterocyclic compound represented by Chemical Formula 1. The light emitting auxiliary layer performs a role of increasing light emission efficiency by compensating an optical resonance distance resulting from a wavelength of light emitted from a light emitting layer, and the electron blocking layer may perform a role of preventing electron injection from an electron transport region. In addition, the light emitting auxiliary layer is a layer positioned between a negative electrode and a light emitting layer, or between a positive electrode and a light emitting layer, and when the light emitting auxiliary layer is positioned between the negative electrode and the light emitting layer, the light emitting auxiliary layer may be used to facilitate hole injection and/or transport, or block electron overflow, and when the light emitting auxiliary layer is positioned between the positive electrode and the light emitting layer, the light emitting auxiliary layer may be used to facilitate electron injection and/or transport, or block hole overflow.

In another embodiment of the present disclosure, the organic material layer may comprise a hole transport layer, and the hole transport layer may comprise the heterocyclic compound represented by Chemical Formula 1.

In another embodiment of the present disclosure, the organic material layer may comprise an electron blocking layer, and the electron blocking layer may comprise the heterocyclic compound represented by Chemical Formula 1.

In another embodiment of the present disclosure, the organic material layer may comprise a light emitting auxiliary layer, and the light emitting auxiliary layer may comprise the heterocyclic compound represented by Chemical Formula 1.

In one embodiment of the present disclosure, the organic light emitting device may be a blue organic light emitting device, and the heterocyclic compound represented by Chemical Formula 1 may be used as a material of the blue organic light emitting device.

In one embodiment of the present disclosure, the organic light emitting device may be a green organic light emitting device, and the heterocyclic compound represented by Chemical Formula 1 may be used as a material of the green organic light emitting device.

In one embodiment of the present disclosure, the organic light emitting device may be a red organic light emitting device, and the heterocyclic compound represented by Chemical Formula 1 may be used as a material of the red organic light emitting device.

In another embodiment of the present disclosure, the organic light emitting device may be a blue organic light emitting device, and the heterocyclic compound represented by Chemical Formula 1 may be used as a light emitting layer material of the blue organic light emitting device.

In another embodiment of the present disclosure, the organic light emitting device may be a green organic light emitting device, and the heterocyclic compound represented by Chemical Formula 1 may be used as a light emitting layer material of the green organic light emitting device.

In another embodiment of the present disclosure, the organic light emitting device may be a red organic light emitting device, and the heterocyclic compound represented by Chemical Formula 1 may be used as a light emitting layer material of the red organic light emitting device.

Specific descriptions on the heterocyclic compound represented by Chemical Formula 1 are the same as the descriptions provided above.

In the organic light emitting device according to one embodiment of the present disclosure, the organic material layer comprises an electron injection layer or an electron transport layer, and the electron injection layer or the electron transport layer may comprise the heterocyclic compound represented by Chemical Formula 1.

In the organic light emitting device according to another embodiment of the present disclosure, the organic material layer comprises an electron blocking layer or a hole blocking layer, and the electron blocking layer or the hole blocking layer may comprise the heterocyclic compound represented by Chemical Formula 1.

In the organic light emitting device according to another embodiment of the present disclosure, the organic material layer comprises an electron transport layer, a light emitting layer or a hole blocking layer, and the electron transport layer, the light emitting layer or the hole blocking layer may comprise the heterocyclic compound represented by Chemical Formula 1.

In the organic light emitting device according to another embodiment of the present disclosure, the organic material layer comprises a hole transport layer, an electron blocking layer or a light emitting auxiliary layer, and the hole transport layer, the electron blocking layer or the light emitting auxiliary layer may comprise the heterocyclic compound represented by Chemical Formula 1.

The organic light emitting device of the present disclosure may be manufactured using common organic light emitting device manufacturing methods and materials except that one or more organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, an electron blocking layer, a hole transport layer, a light emitting layer, an electron transport layer, a hole blocking layer, an electron injection layer, a light emitting auxiliary layer and the like as the organic material layer. However, the structure of the organic light emitting device is not limited thereto, and may include a smaller number of organic material layers.

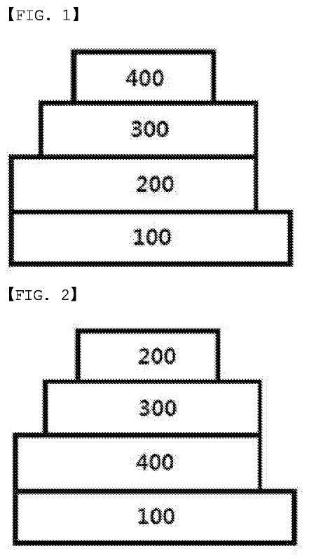

FIG. 1 to FIG. 3 illustrate a lamination order of electrodes and organic material layers of the organic light emitting device according to one embodiment of the present disclosure. However, the scope of the present application is not limited to these diagrams, and structures of organic light emitting devices known in the art may also be used in the present application.

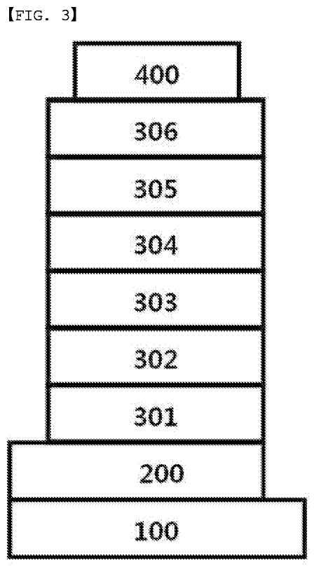

FIG. 1 illustrates the organic light emitting device in which a positive electrode (200), an organic material layer (300) and a negative electrode (400) are consecutively laminated on a substrate (100). However, the structure is not limited to such a structure, and as illustrated in FIG. 2, an organic light emitting device in which a negative electrode, an organic material layer and a positive electrode are consecutively laminated on a substrate may also be obtained.

FIG. 3 illustrates a case of the organic material layer being a multilayer. The organic light emitting device according to FIG. 3 includes a hole injection layer (301), a hole transport layer (302), a light emitting layer (303), a hole blocking layer (304), an electron transport layer (305) and an electron injection layer (306). However, the scope of the present application is not limited to such a lamination structure, and as necessary, the layers other than the light emitting layer may not be included, and other necessary functional layers may be further added. For example, a light emitting auxiliary layer may be added (not shown in FIG. 3).

In addition, one embodiment of the present disclosure provides a composition for an organic material layer of an organic light emitting device, the composition comprising the heterocyclic compound represented by Chemical Formula 1.

Specific descriptions on the heterocyclic compound represented by Chemical Formula 1 are the same as the descriptions provided above.

The composition for an organic material layer of an organic light emitting device may be used when forming an organic material of an organic light emitting device, and particularly, may be more preferably used when forming a hole transport layer, an electron blocking layer or a light emitting auxiliary layer.

One embodiment of the present disclosure provides a method for manufacturing an organic light emitting device, the method comprising the steps of:
preparing a substrate;
forming a first electrode on the substrate;
forming one or more organic material layers on the first electrode; and
forming a second electrode on the one or more organic material layers,
wherein the step of forming the one or more organic material layers includes a step of forming the one or more organic material layers using the composition for an organic material layer according to one embodiment of the present disclosure.

In the organic light emitting device according to one embodiment of the present disclosure, materials other than the heterocyclic compound represented by Chemical Formula 1 are illustrated below, however, these are for illustrative purposes only and not for limiting the scope of the present application, and these materials may be replaced by materials known in the art.

As a positive electrode material, materials having relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As a negative electrode material, materials having relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As a hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tris[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As a hole transport layer material, pyrazoline derivatives, arylamine-based derivatives, stilbene derivatives, triphenyldiamine derivatives and the like may be used, and low molecular or high molecular materials may also be used.

As an electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection layer material, LiF is typically used in the art, however, the present application is not limited thereto.

As a light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light by binding holes and electrons injected from a positive electrode and a negative electrode, respectively, may be used alone, however, materials having a host material and a dopant material involving together in light emission may also be used.

When mixing hosts of the light emitting layer material, same series hosts may be mixed, or different series hosts may be mixed. For example, any two or more types of materials among n-type host materials or p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a similar principle used in the organic light emitting device.

Hereinafter, preferred examples are provided to illuminate the present disclosure, however, the following examples are provided to more readily understand the present disclosure, and the present disclosure is not limited thereto.

### Preparation Example

### Preparation Example 1. Preparation of Compound 20

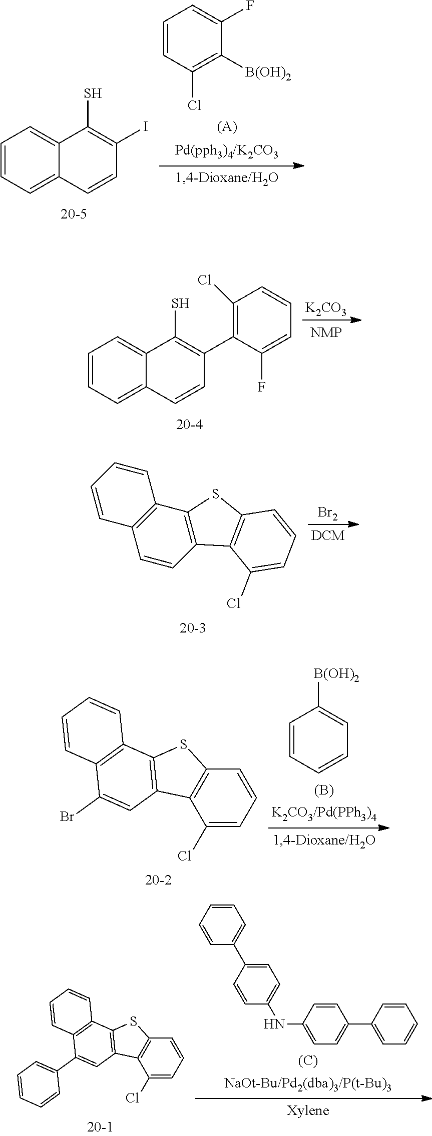

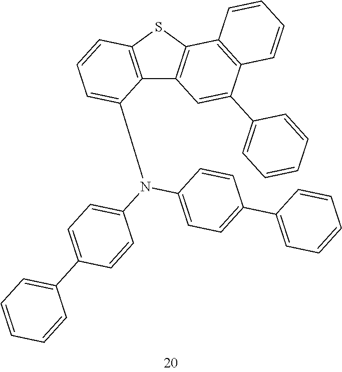 [SMILES: c1ccc(-c2ccc(N(c3ccc(-c4ccccc4)cc3)c3cccc4sc5c6ccccc6c(-c6ccccc6)cc5c34)cc2)cc1]

### Preparation Example 1-1. Preparation of Compound 20-4

Compound 20-5 (50 μg, 174.75 mmol), (2-chloro-6-fluorophenyl)boronic acid (A) (34.2 g, 192.22 mmol), tetrakis(triphenylphosphine) palladium(0) (Pd(PPh₃)₄) (201.93 g, 174.75 mmol) and potassium carbonate (K₂CO₃) (24.15 g, 174.75 mmol) were added to 1,4-dioxane (750 mL) and distilled water (150 mL), and stirred for 4 hours at 120° C. After that, the temperature was lowered to room temperature, the water layer was separated, and the organic layer was washed once more with distilled water again to separate the organic layer. Anhydrous magnesium sulfate (MgSO₄) was introduced to the organic layer, and the result was slurried, then filtered, and concentrated under a reduced pressure. The compound in an oil state was separated through silica chromatography using hexane and ethyl acetate to obtain Compound 20-4 (3-chloro-2-(1-fluoro-2-naphthyl)benzenethiol) (46 g, 159.3 mmol, yield 91%).

### Preparation Example 1-2. Preparation of Compound 20-3

Compound 20-4 (46 g, 159.3 mmol) and potassium carbonate (K₂CO₃) (66.04 g, 477.89 mmol) were added to N-methylpyrrolidone (NMP) (460 mL), and stirred for 3 hours at 140° C. After approximately 1 hour, the reaction material was cooled to room temperature, and then slowly introduced to distilled water (500 mL). Precipitated solids were filtered, dissolved in tetrahydrofuran (THF), and, after introducing anhydrous magnesium sulfate (MgSO₄) thereto, the result was filtered and then concentrated under a reduced pressure. The concentrated compound was slurried with a small amount of tetrahydrofuran and an excess of hexane, and filtered. For purification, the filtered compound was separated through silica chromatography using hexane and ethyl acetate to obtain Compound 20-3 (7-chloronaphtho[1,2-b]benzothiophene) (40 g, 148.83 mmol, yield 93.431%).

### Preparation Example 1-3. Preparation of Compound 20-2

Compound 20-3 (40 g, 148.83 mmol) was dissolved in dichloromethane (DCM) (400 mL), and then stirred in an ice bath. Bromine (26.43 g, 163.71 mmol) was added dropwise thereto using a needle, and the result was stirred for 3 hours at room temperature to obtain Compound 20-2 (5-bromo-7-chloro-naphtho[1,2-b]benzothiophene) (51 g, 146.7 mmol, yield 98.564%).

### Preparation Example 1-4. Preparation of Compound 20-1

Compound 20-2 (51 g, 146.7 mmol), phenyl boronic acid (B) (21.9 g, 176.03 mmol), tetrakis(triphenylphosphine) palladium(0) (Pd(PPh₃)₄) (8.48 g, 7.33 mmol) and potassium carbonate (K₂CO₃) (60.82 g, 440.09 mmol) were added to 1,4-dioxane (1000 mL) and distilled water (200 mL), and reacted under reflux for 5 hours. After the reaction was completed, the reaction solution was reacted by introducing methylene chloride (MC) (100 mL) and distilled water (150 mL) thereto, and then introduced to a separatory funnel to separate the organic layer. The organic layer was dried by introducing anhydrous magnesium sulfate (MgSO₄) thereto, and after removing the solvent using a rotary evaporator, the result was slurried with acetone and hexane to obtain Compound 20-1 (7-chloro-5-phenyl-naphtho[1,2-b]benzothiophene) (50 g, 144.99 mmol, yield 98.835%).

### Preparation Example 1-5. Preparation of Compound 20

Compound 20-1 (50 g, 144.99 mmol), bis(4-biphenylyl)amine (C) (52.31 g, 159.49 mol), tris(dibenzylideneacetone) dipalladium (Pd₂(dba)₃) (6.64 g, 7.25 mmol), dicyclohexyl(2′,4′,6′-triisopropyl-[1,1′-biphenyl]-2-yl)phosphine (Xphos) (6.91 g, 14.5 mmol) and sodium t-butoxide (t-BuONa) (41.8 g, 434.96 mmol) were introduced to xylene (1000 mL), and stirred under reflux for hours at 125° C. After the reaction was completed, the reaction solution was dissolved by introducing methylene chloride (MC) thereto, and then extracted with distilled water. The organic layer was dried by introducing anhydrous magnesium sulfate (MgSO₄) thereto, and after removing the solvent using a rotary evaporator, the result was purified by column chromatography (MC:hexane=1:3) to obtain Compound 20 (78 g, 123.85 mmol, yield 85.42%).

Target compounds were synthesized as in the following Table 1 by preparing in the same manner as in Preparation Example 1 except that Intermediate A was used instead of (2-chloro-6-fluorophenyl)boronic acid, Intermediate B was used instead of phenylboronic acid, and Intermediate C was used instead of bis(4-biphenylyl)amine.

TABLE 1

| Com- | | | | | |
|---|---|---|---|---|---|
| pound | | | | | |
| No. | Intermediate A | Intermediate B | Intermediate C | Target Compound | Yield |
| 24 | 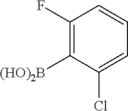 [SMILES: OB(O)c1c(F)cccc1Cl] | 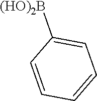 | 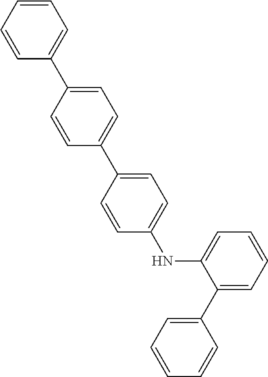 [SMILES: c1ccc(-c2ccc(-c3ccc(Nc4ccccc4-c4ccccc4)cc3)cc2)cc1] | 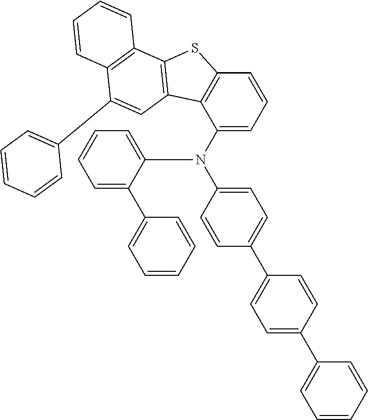 [SMILES: c1ccc(-c2ccc(-c3ccc(N(c4ccccc4-c4ccccc4)c4cccc5sc6c7ccccc7c(-c7ccccc7)cc6c45)cc3)cc2)cc1] | 56% |
| 32 | 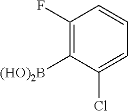 [SMILES: OB(O)c1c(F)cccc1Cl] | 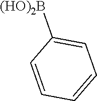 | 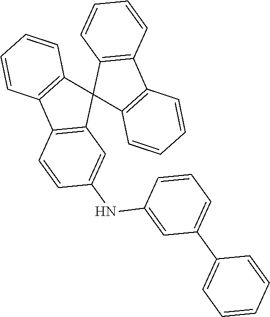 [SMILES: c1ccc(-c2cccc(Nc3ccc4c(c3)C3(c5ccccc5-c5ccccc53)c3ccccc3-4)c2)cc1] | 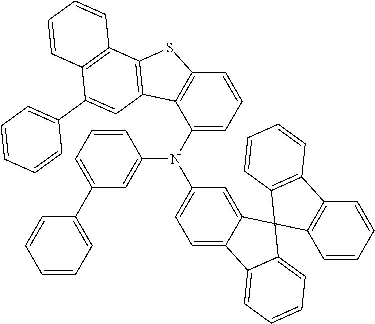 [SMILES: c1ccc(-c2cccc(N(c3ccc4c(c3)C3(c5ccccc5-c5ccccc53)c3ccccc3-4)c3cccc4sc5c6ccccc6c(-c6ccccc6)cc5c34)c2)cc1] | 65% |
| 40 | 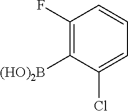 [SMILES: OB(O)c1c(F)cccc1Cl] | 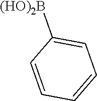 | 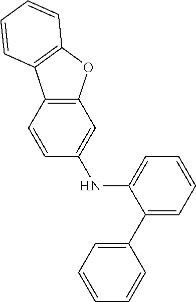 [SMILES: c1ccc(-c2ccccc2Nc2ccc3c(c2)oc2ccccc23)cc1] | 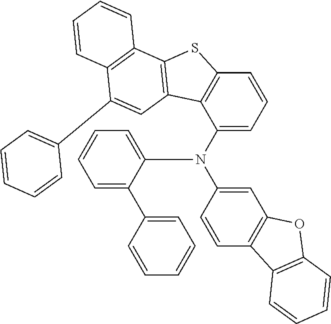 [SMILES: c1ccc(-c2ccccc2N(c2ccc3c(c2)oc2ccccc23)c2cccc3sc4c5ccccc5c(-c5ccccc5)cc4c23)cc1] | 78% |
| 48 | 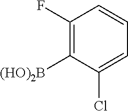 [SMILES: OB(O)c1c(F)cccc1Cl] | 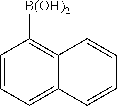 [SMILES: OB(O)c1cccc2ccccc12] | 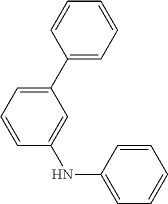 [SMILES: c1ccc(Nc2cccc(-c3ccccc3)c2)cc1] | 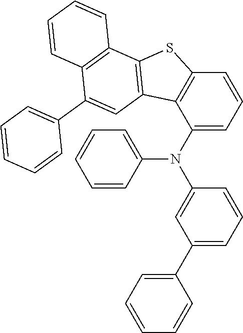 [SMILES: c1ccc(-c2cccc(N(c3ccccc3)c3cccc4sc5c6ccccc6c(-c6ccccc6)cc5c34)c2)cc1] | 58% |
| 56 | 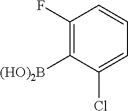 [SMILES: OB(O)c1c(F)cccc1Cl] | 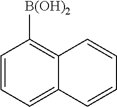 [SMILES: OB(O)c1cccc2ccccc12] | 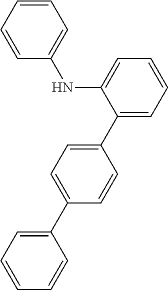 [SMILES: c1ccc(Nc2ccccc2-c2ccc(-c3ccccc3)cc2)cc1] | 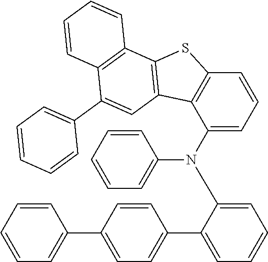 [SMILES: c1ccc(-c2ccc(-c3ccccc3N(c3ccccc3)c3cccc4sc5c6ccccc6c(-c6ccccc6)cc5c34)cc2)cc1] | 68% |
| 64 | 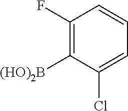 [SMILES: OB(O)c1c(F)cccc1Cl] | 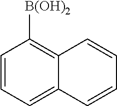 [SMILES: OB(O)c1cccc2ccccc12] | 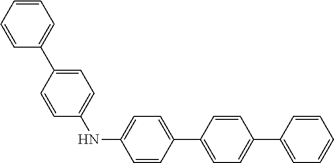 [SMILES: c1ccc(-c2ccc(Nc3ccc(-c4ccc(-c5ccccc5)cc4)cc3)cc2)cc1] | 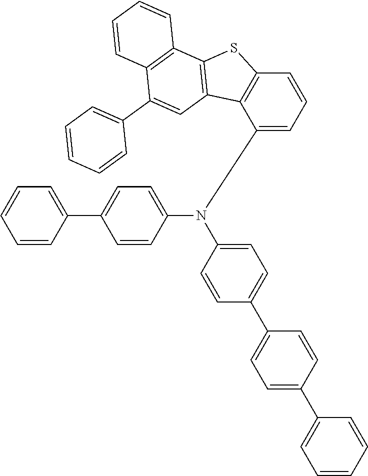 [SMILES: c1ccc(-c2ccc(-c3ccc(N(c4ccc(-c5ccccc5)cc4)c4cccc5sc6c7ccccc7c(-c7ccccc7)cc6c45)cc3)cc2)cc1] | 75% |
| 71 | 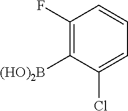 [SMILES: OB(O)c1c(F)cccc1Cl] | 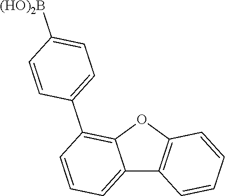 [SMILES: OB(O)c1ccc(-c2cccc3c2oc2ccccc23)cc1] | 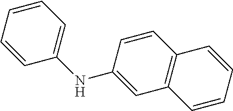 [SMILES: c1ccc(Nc2ccc3ccccc3c2)cc1] | 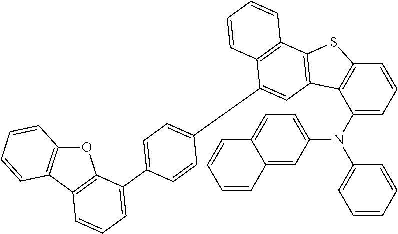 [SMILES: c1ccc(N(c2ccc3ccccc3c2)c2cccc3sc4c5ccccc5c(-c5ccc(-c6cccc7c6oc6ccccc67)cc5)cc4c23)cc1] | 73% |
| 72 | 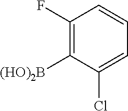 [SMILES: OB(O)c1c(F)cccc1Cl] | 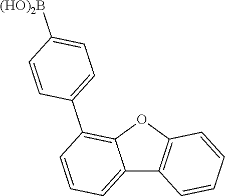 [SMILES: OB(O)c1ccc(-c2cccc3c2oc2ccccc23)cc1] | 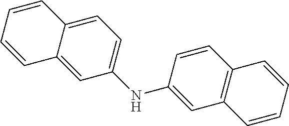 [SMILES: c1ccc2cc(Nc3ccc4ccccc4c3)ccc2c1] | 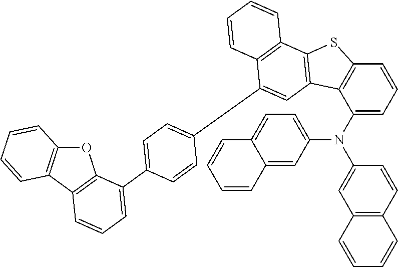 [SMILES: c1ccc2cc(N(c3ccc4ccccc4c3)c3cccc4sc5c6ccccc6c(-c6ccc(-c7cccc8c7oc7ccccc78)cc6)cc5c34)ccc2c1] | 83% |
| 80 | 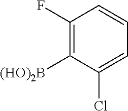 [SMILES: OB(O)c1c(F)cccc1Cl] | 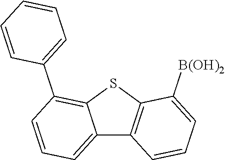 [SMILES: OB(O)c1cccc2c1sc1c(-c3ccccc3)cccc12] | 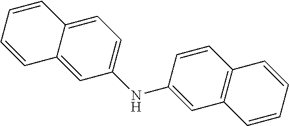 [SMILES: c1ccc2cc(Nc3ccc4ccccc4c3)ccc2c1] | 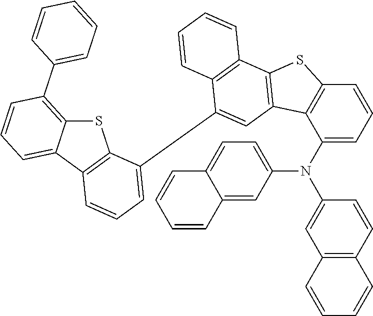 [SMILES: c1ccc(-c2cccc3c2sc2c(-c4cc5c(sc6cccc(N(c7ccc8ccccc8c7)c7ccc8ccccc8c7)c65)c5ccccc45)cccc23)cc1] | 76% |
| 96 | 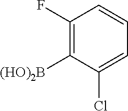 [SMILES: OB(O)c1c(F)cccc1Cl] | 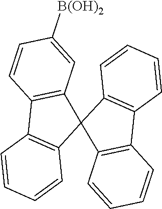 [SMILES: OB(O)c1ccc2c(c1)C1(c3ccccc3-c3ccccc31)c1ccccc1-2] | 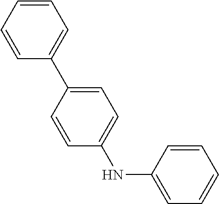 [SMILES: c1ccc(Nc2ccc(-c3ccccc3)cc2)cc1] | 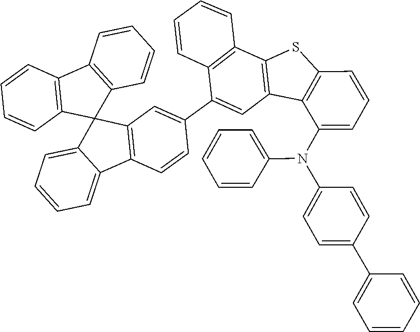 [SMILES: c1ccc(-c2ccc(N(c3ccccc3)c3cccc4sc5c6ccccc6c(-c6ccc7c(c6)C6(c8ccccc8-c8ccccc86)c6ccccc6-7)cc5c34)cc2)cc1] | 75% |
| 98 | 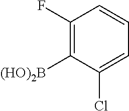 [SMILES: OB(O)c1c(F)cccc1Cl] | 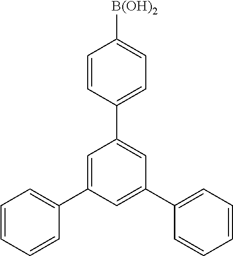 [SMILES: OB(O)c1ccc(-c2cc(-c3ccccc3)cc(-c3ccccc3)c2)cc1] | 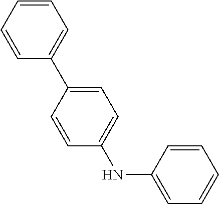 [SMILES: c1ccc(Nc2ccc(-c3ccccc3)cc2)cc1] | 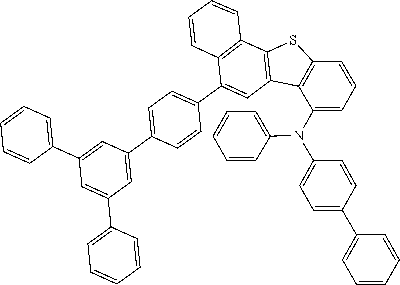 [SMILES: c1ccc(-c2ccc(N(c3ccccc3)c3cccc4sc5c6ccccc6c(-c6ccc(-c7cc(-c8ccccc8)cc(-c8ccccc8)c7)cc6)cc5c34)cc2)cc1] | 85% |
| 120 | 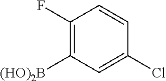 [SMILES: OB(O)c1cc(Cl)ccc1F] | 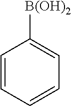 | 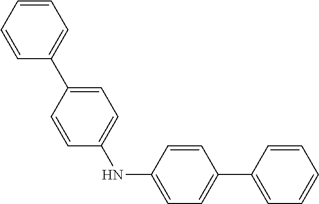 [SMILES: c1ccc(-c2ccc(Nc3ccc(-c4ccccc4)cc3)cc2)cc1] | 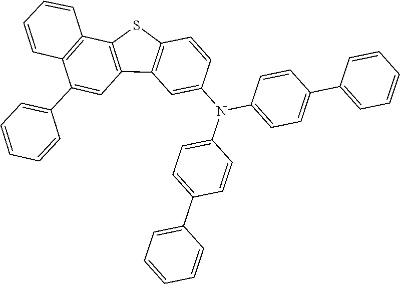 [SMILES: c1ccc(-c2ccc(N(c3ccc(-c4ccccc4)cc3)c3ccc4sc5c6ccccc6c(-c6ccccc6)cc5c4c3)cc2)cc1] | 81% |
| 138 | 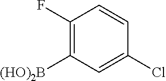 [SMILES: OB(O)c1cc(Cl)ccc1F] | 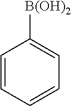 | 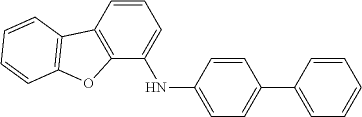 [SMILES: c1ccc(-c2ccc(Nc3cccc4c3oc3ccccc34)cc2)cc1] | 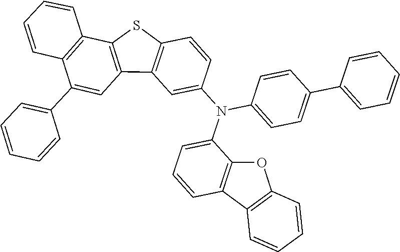 [SMILES: c1ccc(-c2ccc(N(c3ccc4sc5c6ccccc6c(-c6ccccc6)cc5c4c3)c3cccc4c3oc3ccccc34)cc2)cc1] | 61% |
| 141 | 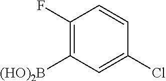 [SMILES: OB(O)c1cc(Cl)ccc1F] | 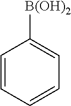 | 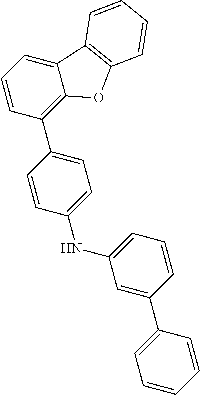 [SMILES: c1ccc(-c2cccc(Nc3ccc(-c4cccc5c4oc4ccccc45)cc3)c2)cc1] | 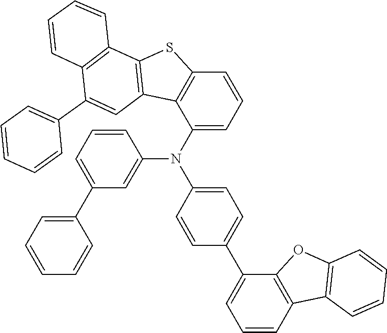 [SMILES: c1ccc(-c2cccc(N(c3ccc(-c4cccc5c4oc4ccccc45)cc3)c3cccc4sc5c6ccccc6c(-c6ccccc6)cc5c34)c2)cc1] | 62% |
| 179 | 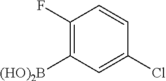 [SMILES: OB(O)c1cc(Cl)ccc1F] | 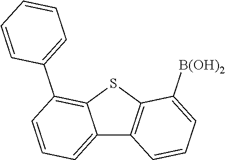 [SMILES: OB(O)c1cccc2c1sc1c(-c3ccccc3)cccc12] | 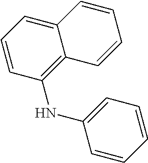 [SMILES: c1ccc(Nc2cccc3ccccc23)cc1] | 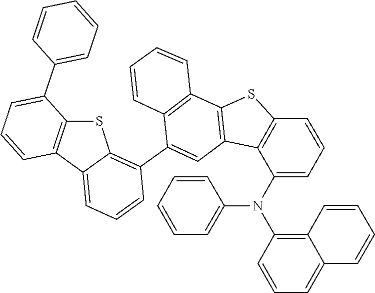 [SMILES: c1ccc(-c2cccc3c2sc2c(-c4cc5c(sc6cccc(N(c7ccccc7)c7cccc8ccccc78)c65)c5ccccc45)cccc23)cc1] | 52% |
| 191 | 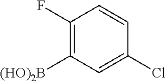 [SMILES: OB(O)c1cc(Cl)ccc1F] | 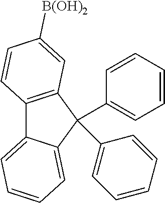 [SMILES: OB(O)c1ccc2c(c1)C(c1ccccc1)(c1ccccc1)c1ccccc1-2] | 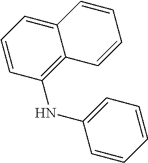 [SMILES: c1ccc(Nc2cccc3ccccc23)cc1] | 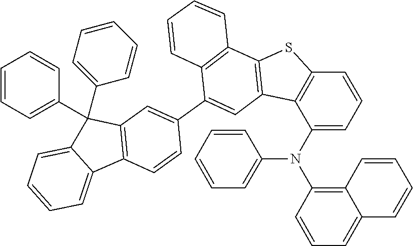 [SMILES: c1ccc(N(c2cccc3ccccc23)c2cccc3sc4c5ccccc5c(-c5ccc6c(c5)C(c5ccccc5)(c5ccccc5)c5ccccc5-6)cc4c23)cc1] | 76% |
| 220 | 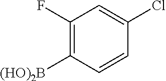 [SMILES: OB(O)c1ccc(Cl)cc1F] | 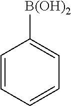 | 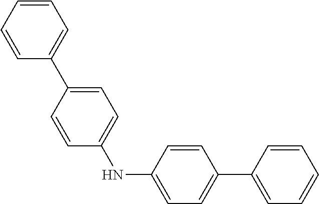 [SMILES: c1ccc(-c2ccc(Nc3ccc(-c4ccccc4)cc3)cc2)cc1] | 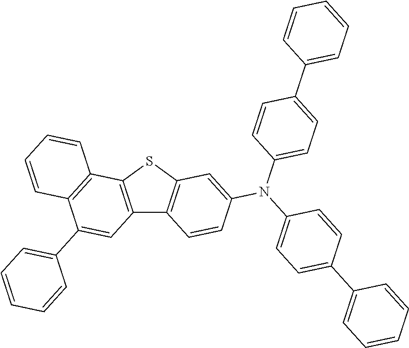 [SMILES: c1ccc(-c2ccc(N(c3ccc(-c4ccccc4)cc3)c3ccc4c(c3)sc3c5ccccc5c(-c5ccccc5)cc43)cc2)cc1] | 55% |
| 228 | 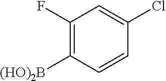 [SMILES: OB(O)c1ccc(Cl)cc1F] | 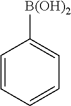 | 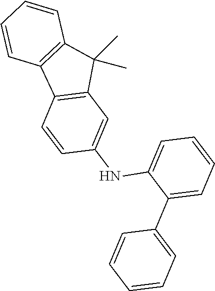 [SMILES: CC1(C)c2ccccc2-c2ccc(Nc3ccccc3-c3ccccc3)cc21] | 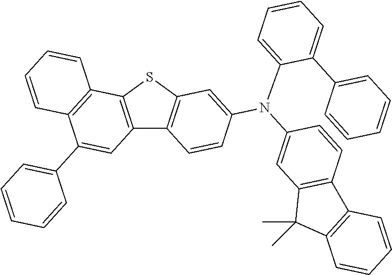 [SMILES: CC1(C)c2ccccc2-c2ccc(N(c3ccc4c(c3)sc3c5ccccc5c(-c5ccccc5)cc43)c3ccccc3-c3ccccc3)cc21] | 67% |
| 236 | 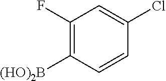 [SMILES: OB(O)c1ccc(Cl)cc1F] | 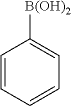 | 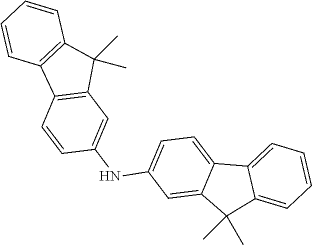 [SMILES: CC1(C)c2ccccc2-c2ccc(Nc3ccc4c(c3)C(C)(C)c3ccccc3-4)cc21] | 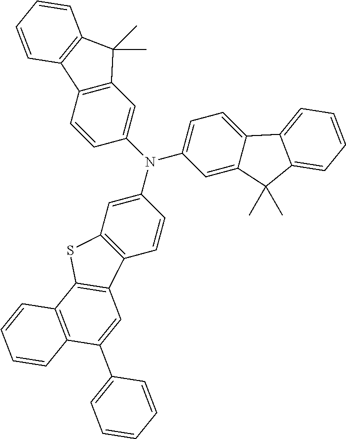 [SMILES: CC1(C)c2ccccc2-c2ccc(N(c3ccc4c(c3)C(C)(C)c3ccccc3-4)c3ccc4c(c3)sc3c5ccccc5c(-c5ccccc5)cc43)cc21] | 87% |
| 243 | 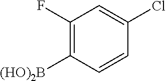 [SMILES: OB(O)c1ccc(Cl)cc1F] | 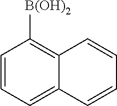 [SMILES: OB(O)c1cccc2ccccc12] | 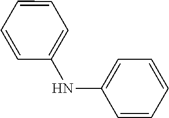 [SMILES: c1ccc(Nc2ccccc2)cc1] | 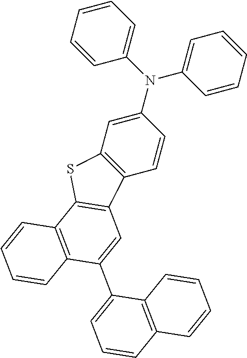 [SMILES: c1ccc(N(c2ccccc2)c2ccc3c(c2)sc2c4ccccc4c(-c4cccc5ccccc45)cc32)cc1] | 77% |
| 251 | 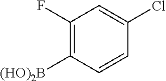 [SMILES: OB(O)c1ccc(Cl)cc1F] | 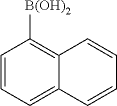 [SMILES: OB(O)c1cccc2ccccc12] | 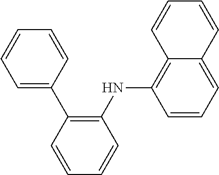 [SMILES: c1ccc(-c2ccccc2Nc2cccc3ccccc23)cc1] | 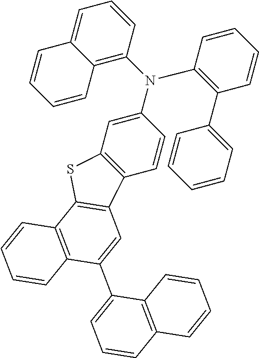 [SMILES: c1ccc(-c2ccccc2N(c2ccc3c(c2)sc2c4ccccc4c(-c4cccc5ccccc45)cc32)c2cccc3ccccc23)cc1] | 67% |
| 259 | 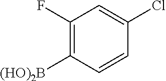 [SMILES: OB(O)c1ccc(Cl)cc1F] | 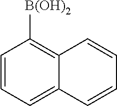 [SMILES: OB(O)c1cccc2ccccc12] | 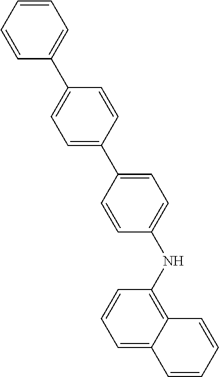 [SMILES: c1ccc(-c2ccc(-c3ccc(Nc4cccc5ccccc45)cc3)cc2)cc1] | 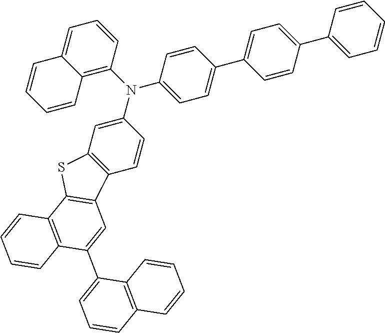 [SMILES: c1ccc(-c2ccc(-c3ccc(N(c4ccc5c(c4)sc4c6ccccc6c(-c6cccc7ccccc67)cc54)c4cccc5ccccc45)cc3)cc2)cc1] | 63% |
| 292 | 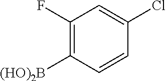 [SMILES: OB(O)c1ccc(Cl)cc1F] | 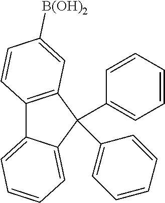 [SMILES: OB(O)c1ccc2c(c1)C(c1ccccc1)(c1ccccc1)c1ccccc1-2] | 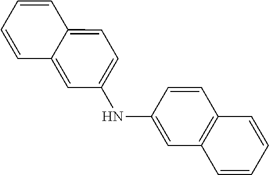 [SMILES: c1ccc2cc(Nc3ccc4ccccc4c3)ccc2c1] | 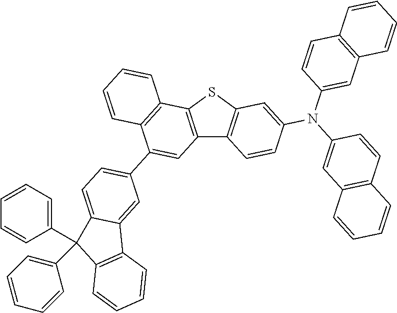 [SMILES: c1ccc(C2(c3ccccc3)c3ccccc3-c3cc(-c4cc5c6ccc(N(c7ccc8ccccc8c7)c7ccc8ccccc8c7)cc6sc5c5ccccc45)ccc32)cc1] | 52% |
| 332 | 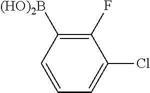 [SMILES: OB(O)c1cccc(Cl)c1F] | 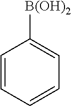 | 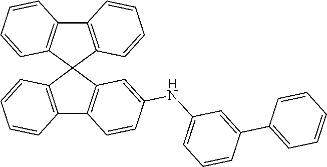 [SMILES: c1ccc(-c2cccc(Nc3ccc4c(c3)C3(c5ccccc5-c5ccccc53)c3ccccc3-4)c2)cc1] | 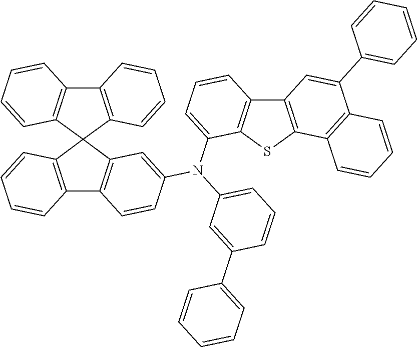 [SMILES: c1ccc(-c2cccc(N(c3ccc4c(c3)C3(c5ccccc5-c5ccccc53)c3ccccc3-4)c3cccc4c3sc3c5ccccc5c(-c5ccccc5)cc43)c2)cc1] | 71% |
| 377 | 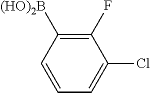 [SMILES: OB(O)c1cccc(Cl)c1F] | 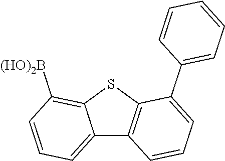 [SMILES: OB(O)c1cccc2c1sc1c(-c3ccccc3)cccc12] | 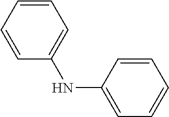 [SMILES: c1ccc(Nc2ccccc2)cc1] | 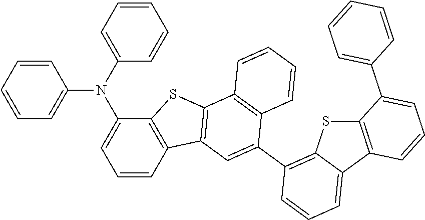 [SMILES: c1ccc(-c2cccc3c2sc2c(-c4cc5c6cccc(N(c7ccccc7)c7ccccc7)c6sc5c5ccccc45)cccc23)cc1] | 47% |
| 684 | 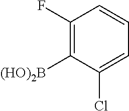 [SMILES: OB(O)c1c(F)cccc1Cl] | 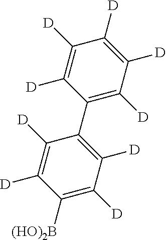 [SMILES: [2H]c1c([2H])c([2H])c(-c2c([2H])c([2H])c(B(O)O)c([2H])c2[2H])c([2H])c1[2H]] | 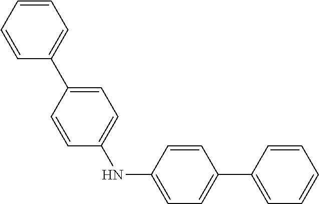 [SMILES: c1ccc(-c2ccc(Nc3ccc(-c4ccccc4)cc3)cc2)cc1] | 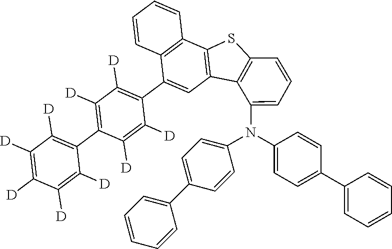 [SMILES: [2H]c1c([2H])c([2H])c(-c2c([2H])c([2H])c(-c3cc4c(sc5cccc(N(c6ccc(-c7ccccc7)cc6)c6ccc(-c7ccccc7)cc6)c54)c4ccccc34)c([2H])c2[2H])c([2H])c1[2H]] | 47% |
| 688 | 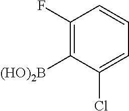 [SMILES: OB(O)c1c(F)cccc1Cl] | 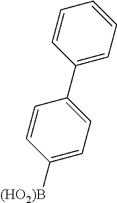 | 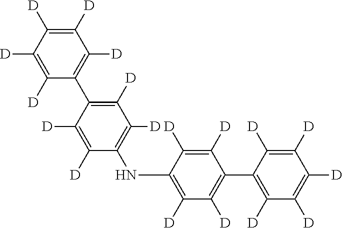 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 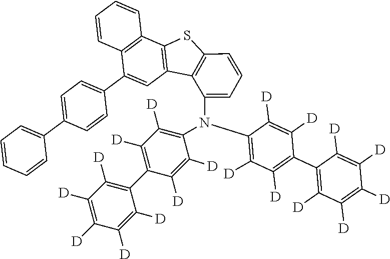 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cccc4sc5c6ccccc6c(-c6ccc(-c7ccccc7)cc6)cc5c34)c([2H])c2[2H])c([2H])c1[2H]] | 77% |
| 693 | 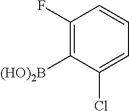 [SMILES: OB(O)c1c(F)cccc1Cl] | 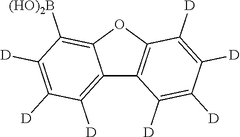 [SMILES: [2H]c1c([2H])c([2H])c2c(oc3c(B(O)O)c([2H])c([2H])c([2H])c32)c1[2H]] | 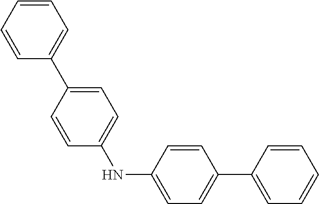 [SMILES: c1ccc(-c2ccc(Nc3ccc(-c4ccccc4)cc3)cc2)cc1] | 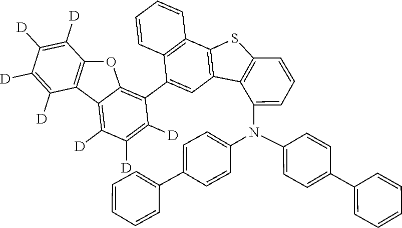 [SMILES: [2H]c1c([2H])c([2H])c2c(oc3c(-c4cc5c(sc6cccc(N(c7ccc(-c8ccccc8)cc7)c7ccc(-c8ccccc8)cc7)c65)c5ccccc45)c([2H])c([2H])c([2H])c32)c1[2H]] | 83% |

### Preparation Example 2. Preparation of Compound 420

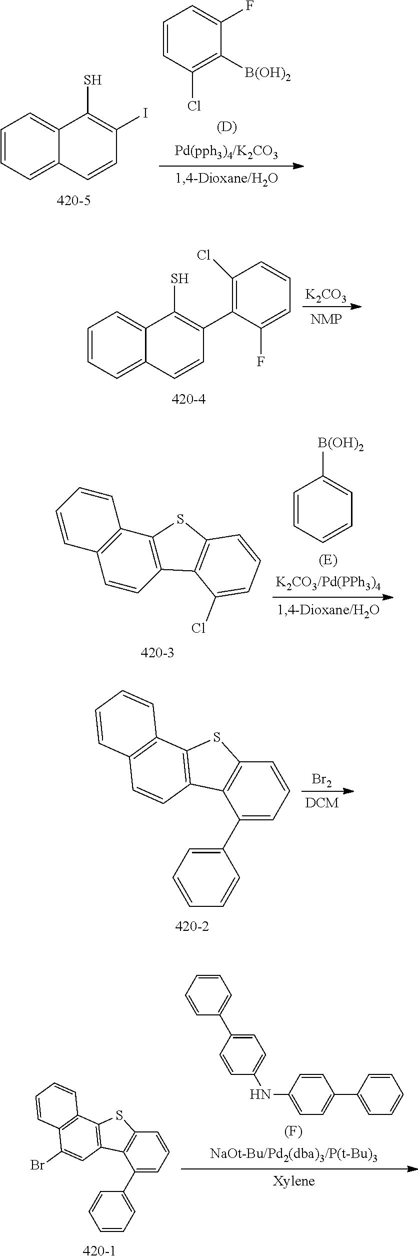

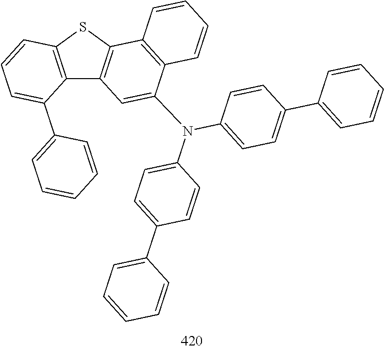 [SMILES: c1ccc(-c2ccc(N(c3ccc(-c4ccccc4)cc3)c3cc4c(sc5cccc(-c6ccccc6)c54)c4ccccc34)cc2)cc1]

### Preparation Example 2-1. Preparation of Compound 420-4

Compound 420-5 (50 g, 174.75 mmol), (2-chloro-6-fluorophenyl)boronic acid (D) (34.2 g, 192.22 mmol), tetrakis(triphenylphosphine) palladium(0) (Pd(PPh₃)₄) (201.93 g, 174.75 mmol) and potassium carbonate (K₂CO₃) (24.15 g, 174.75 mmol) were added to 1,4-dioxane (750 mL) and distilled water (150 mL), and stirred for 4 hours at 120° C. After that, the temperature was lowered to room temperature, the water layer was separated, and the organic layer was washed once more with distilled water again to separate the organic layer. Anhydrous magnesium sulfate (MgSO₄) was introduced to the organic layer, and the result was slurried, then filtered, and concentrated under a reduced pressure. The compound in an oil state was separated through silica chromatography using hexane and ethyl acetate to obtain Compound 420-4 (3-chloro-2-(1-fluoro-2-naphthyl)benzenethiol) (46 g, 159.3 mmol, yield 91%).

### Preparation Example 2-2. Preparation of Compound 420-3

Compound 420-4 (46 g, 159.3 mmol) and potassium carbonate (K₂CO₃) (66.04 g, 477.89 mmol) were added to N-methylpyrrolidone (NMP) (460 mL), and stirred for 3 hours at 140° C. After approximately 1 hour, the reaction material was cooled to room temperature, and then slowly introduced to distilled water (500 mL). Precipitated solids were filtered, dissolved in tetrahydrofuran (THF), and, after introducing anhydrous magnesium sulfate (MgSO₄) thereto, the result was filtered and then concentrated under a reduced pressure. The concentrated compound was slurried with a small amount of tetrahydrofuran and an excess of hexane, and filtered. For purification, the filtered compound was separated through silica chromatography using hexane and ethyl acetate to obtain Compound 420-3 (7-chloronaphtho[1,2-b]benzothiophene (40 g, 148.83 mmol, yield 93.431%).

### Preparation Example 2-3. Preparation of Compound 420-2

Compound 420-3 (40 g, 148.83 mmol), phenyl boronic acid (E) (20.37 g, 163.71 mmol), tetrakis(triphenylphosphine)palladium(0) (Pd(PPh₃)₄) (8.6 g, 7.44 mmol) and potassium carbonate (K₂CO₃) (61.71 g, 446.5 mmol) were added to 1,4-dioxane (450 mL) and distilled water (90 mL), and reacted under reflux for 5 hours. After the reaction was completed, the reaction solution was reacted by introducing methylene chloride (MC) (100 mL) and distilled water (150 mL) thereto, and then introduced to a separatory funnel to separate the organic layer. The organic layer was dried by introducing anhydrous magnesium sulfate (MgSO₄) thereto, and after removing the solvent using a rotary evaporator, the result was slurried with acetone and hexane to obtain Compound 420-2 (7-phenylnaphtho[1,2-b]benzothiophene) (45 g, 144.97 mmol, yield 97.405%).

### Preparation Example 2-4. Preparation of Compound 420-1

Compound 420-2 (45 g, 144.97 mmol) was dissolved in dichloromethane (DCM) (400 mL), and then stirred in an ice bath. Bromine (46.8 g, 289.94 mmol) was added dropwise thereto using a needle, and the result was stirred for 3 hours at roan temperature to obtain Compound 420-1 (5-bromo-7-phenyl-naphtho[1,2-b]benzothiophene) (76 g, 195.22 mmol, yield 134.66%).

### Preparation Example 2-5. Preparation of Compound 420

Compound 420-1 (76 g, 195.22 mmol), bis(4-biphenylyl)amine (F) (67.23 g, 204.98 mmol), tris(dibenzylideneacetone)dipalladium (Pd₂(dba)₃) (8.94 g, 9.76 mmol), dicyclohexyl(2′,4′,6′-triisopropyl-[1,1′-biphenyl]-2-yl)phosphine (Xphos) (9.31 g, 19.52 mmol) and sodium t-butoxide (t-BuONa) (56.28 g, 585.65 mmol) were introduced to xylene (1400 mL), and stirred under reflux for hours at 125° C. After the reaction was completed, the reaction solution was dissolved by introducing methylene chloride (MC) thereto, and then extracted with distilled water. The organic layer was dried by introducing anhydrous magnesium sulfate (MgSO₄) thereto, and after removing the solvent using a rotary evaporator, the result was purified by column chromatography (MC:hexane=1:3) to obtain Compound 420 (89 g, 141.31 mmol, yield 72.39%).

Target compounds were synthesized as in the following Table 2 by preparing in the same manner as in Preparation Example 2 except that Intermediate D was used instead of (2-chloro-6-fluorophenyl)boronic acid, Intermediate E was used instead of phenylboronic acid, and Intermediate F was used instead of bis(4-biphenylyl)amine.

TABLE 2

| Com | | | | | |
|---|---|---|---|---|---|
| pound | | | | | |
| No. | Intermediate E | Intermediate F | Intermediate G | Target Compound | Yield |
| 432 | 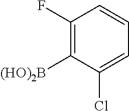 [SMILES: OB(O)c1c(F)cccc1Cl] | 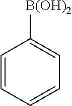 | 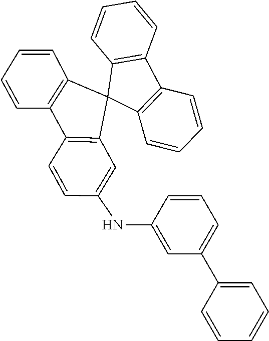 [SMILES: c1ccc(-c2cccc(Nc3ccc4c(c3)C3(c5ccccc5-c5ccccc53)c3ccccc3-4)c2)cc1] | 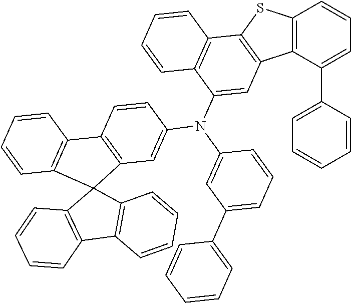 [SMILES: c1ccc(-c2cccc(N(c3ccc4c(c3)C3(c5ccccc5-c5ccccc53)c3ccccc3-4)c3cc4c(sc5cccc(-c6ccccc6)c54)c4ccccc34)c2)cc1] | 69% |
| 440 | 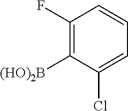 [SMILES: OB(O)c1c(F)cccc1Cl] | 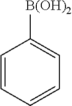 | 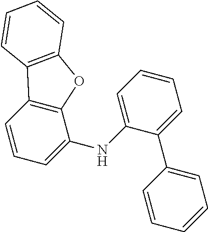 [SMILES: c1ccc(-c2ccccc2Nc2cccc3c2oc2ccccc23)cc1] | 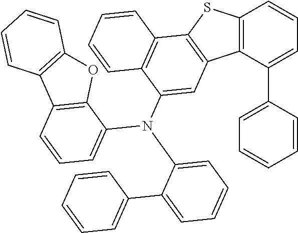 [SMILES: c1ccc(-c2ccccc2N(c2cc3c(sc4cccc(-c5ccccc5)c43)c3ccccc23)c2cccc3c2oc2ccccc23)cc1] | 54% |
| 447 | 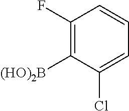 [SMILES: OB(O)c1c(F)cccc1Cl] | 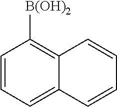 [SMILES: OB(O)c1cccc2ccccc12] | 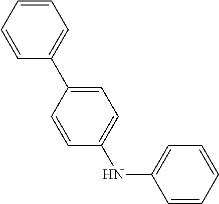 [SMILES: c1ccc(Nc2ccc(-c3ccccc3)cc2)cc1] | 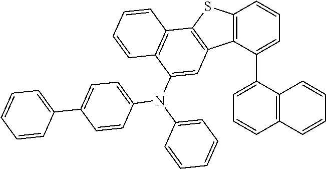 [SMILES: c1ccc(-c2ccc(N(c3ccccc3)c3cc4c(sc5cccc(-c6cccc7ccccc67)c54)c4ccccc34)cc2)cc1] | 67% |
| 455 | 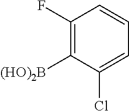 [SMILES: OB(O)c1c(F)cccc1Cl] | 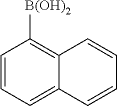 [SMILES: OB(O)c1cccc2ccccc12] | 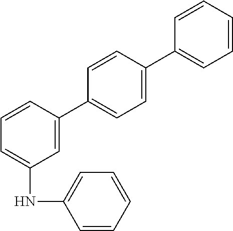 [SMILES: c1ccc(Nc2cccc(-c3ccc(-c4ccccc4)cc3)c2)cc1] | 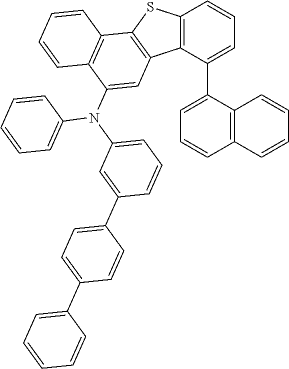 [SMILES: c1ccc(-c2ccc(-c3cccc(N(c4ccccc4)c4cc5c(sc6cccc(-c7cccc8ccccc78)c65)c5ccccc45)c3)cc2)cc1] | 61% |
| 467 | 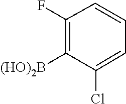 [SMILES: OB(O)c1c(F)cccc1Cl] | 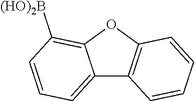 [SMILES: OB(O)c1cccc2c1oc1ccccc12] | 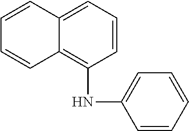 [SMILES: c1ccc(Nc2cccc3ccccc23)cc1] | 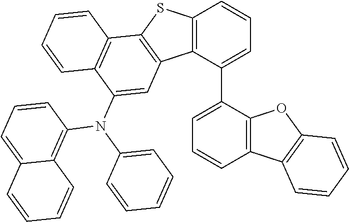 [SMILES: c1ccc(N(c2cccc3ccccc23)c2cc3c(sc4cccc(-c5cccc6c5oc5ccccc56)c43)c3ccccc23)cc1] | 51% |
| 475 | 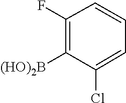 [SMILES: OB(O)c1c(F)cccc1Cl] | 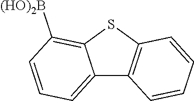 [SMILES: OB(O)c1cccc2c1sc1ccccc12] | 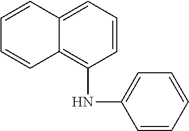 [SMILES: c1ccc(Nc2cccc3ccccc23)cc1] | 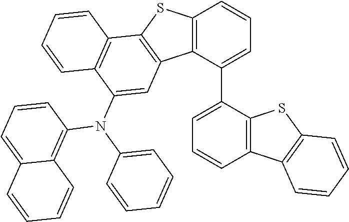 [SMILES: c1ccc(N(c2cccc3ccccc23)c2cc3c(sc4cccc(-c5cccc6c5sc5ccccc56)c43)c3ccccc23)cc1] | 57% |
| 487 | 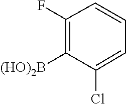 [SMILES: OB(O)c1c(F)cccc1Cl] | 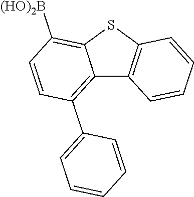 [SMILES: OB(O)c1ccc(-c2ccccc2)c2c1sc1ccccc12] | 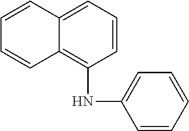 [SMILES: c1ccc(Nc2cccc3ccccc23)cc1] | 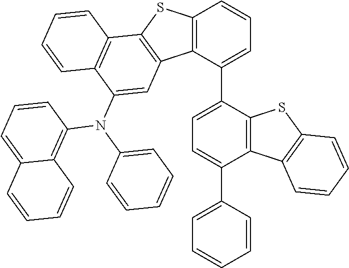 [SMILES: c1ccc(-c2ccc(-c3cccc4sc5c6ccccc6c(N(c6ccccc6)c6cccc7ccccc67)cc5c34)c3sc4ccccc4c23)cc1] | 67% |
| 495 | 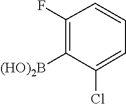 [SMILES: OB(O)c1c(F)cccc1Cl] | 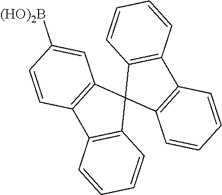 [SMILES: OB(O)c1ccc2c(c1)C1(c3ccccc3-c3ccccc31)c1ccccc1-2] | 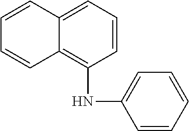 [SMILES: c1ccc(Nc2cccc3ccccc23)cc1] | 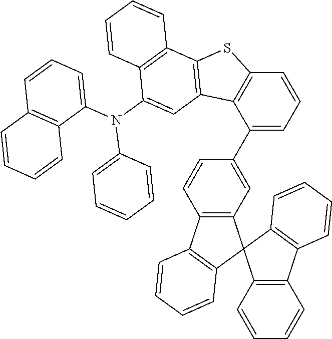 [SMILES: c1ccc(N(c2cccc3ccccc23)c2cc3c(sc4cccc(-c5ccc6c(c5)C5(c7ccccc7-c7ccccc75)c5ccccc5-6)c43)c3ccccc23)cc1] | 79% |
| 507 | 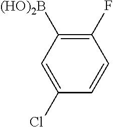 [SMILES: OB(O)c1cc(Cl)ccc1F] | 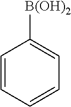 | 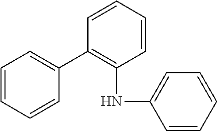 [SMILES: c1ccc(Nc2ccccc2-c2ccccc2)cc1] | 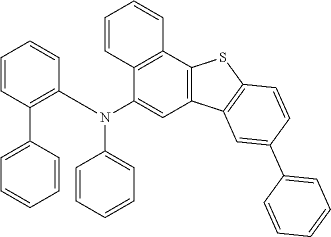 [SMILES: c1ccc(-c2ccc3sc4c5ccccc5c(N(c5ccccc5)c5ccccc5-c5ccccc5)cc4c3c2)cc1] | 79% |
| 515 | 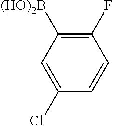 [SMILES: OB(O)c1cc(Cl)ccc1F] | 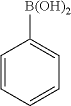 | 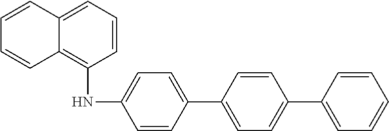 [SMILES: c1ccc(-c2ccc(-c3ccc(Nc4cccc5ccccc45)cc3)cc2)cc1] | 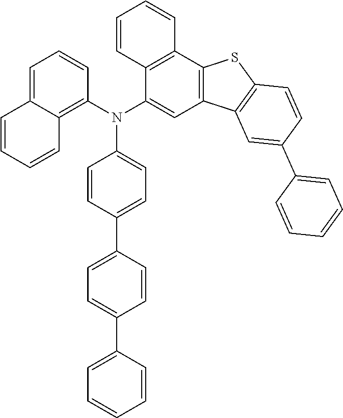 [SMILES: c1ccc(-c2ccc(-c3ccc(N(c4cccc5ccccc45)c4cc5c6cc(-c7ccccc7)ccc6sc5c5ccccc45)cc3)cc2)cc1] | 67% |
| 531 | 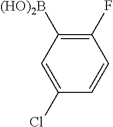 [SMILES: OB(O)c1cc(Cl)ccc1F] | 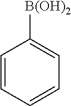 | 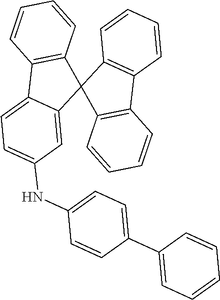 [SMILES: c1ccc(-c2ccc(Nc3ccc4c(c3)C3(c5ccccc5-c5ccccc53)c3ccccc3-4)cc2)cc1] | 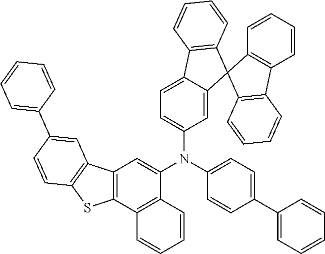 [SMILES: c1ccc(-c2ccc(N(c3ccc4c(c3)C3(c5ccccc5-c5ccccc53)c3ccccc3-4)c3cc4c5cc(-c6ccccc6)ccc5sc4c4ccccc34)cc2)cc1] | 87% |
| 543 | 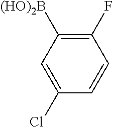 [SMILES: OB(O)c1cc(Cl)ccc1F] | 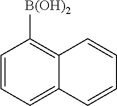 [SMILES: OB(O)c1cccc2ccccc12] | 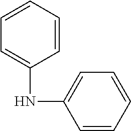 [SMILES: c1ccc(Nc2ccccc2)cc1] | 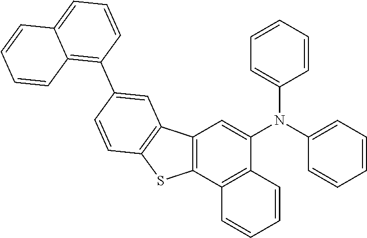 [SMILES: c1ccc(N(c2ccccc2)c2cc3c4cc(-c5cccc6ccccc56)ccc4sc3c3ccccc23)cc1] | 82% |
| 555 | 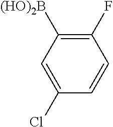 [SMILES: OB(O)c1cc(Cl)ccc1F] | 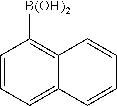 [SMILES: OB(O)c1cccc2ccccc12] | 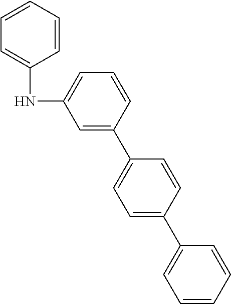 [SMILES: c1ccc(Nc2cccc(-c3ccc(-c4ccccc4)cc3)c2)cc1] | 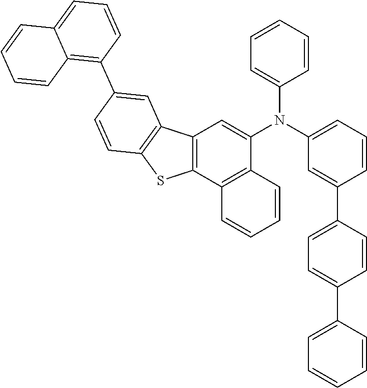 [SMILES: c1ccc(-c2ccc(-c3cccc(N(c4ccccc4)c4cc5c6cc(-c7cccc8ccccc78)ccc6sc5c5ccccc45)c3)cc2)cc1] | 63% |
| 565 | 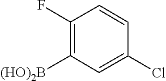 [SMILES: OB(O)c1cc(Cl)ccc1F] | 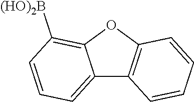 [SMILES: OB(O)c1cccc2c1oc1ccccc12] | 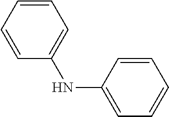 [SMILES: c1ccc(Nc2ccccc2)cc1] | 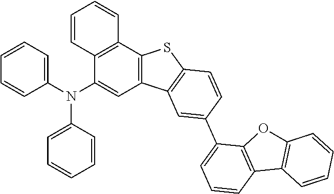 [SMILES: c1ccc(N(c2ccccc2)c2cc3c4cc(-c5cccc6c5oc5ccccc56)ccc4sc3c3ccccc23)cc1] | 69% |
| 579 | 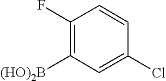 [SMILES: OB(O)c1cc(Cl)ccc1F] | 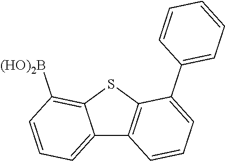 [SMILES: OB(O)c1cccc2c1sc1c(-c3ccccc3)cccc12] | 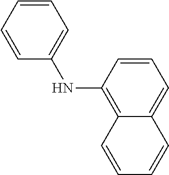 [SMILES: c1ccc(Nc2cccc3ccccc23)cc1] | 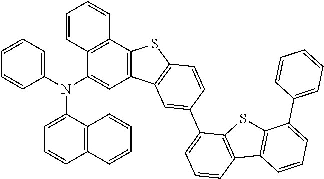 [SMILES: c1ccc(-c2cccc3c2sc2c(-c4ccc5sc6c7ccccc7c(N(c7ccccc7)c7cccc8ccccc78)cc6c5c4)cccc23)cc1] | 72% |
| 595 | 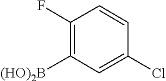 [SMILES: OB(O)c1cc(Cl)ccc1F] | 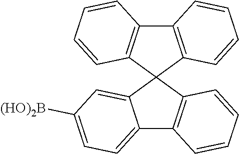 [SMILES: OB(O)c1ccc2c(c1)C1(c3ccccc3-c3ccccc31)c1ccccc1-2] | 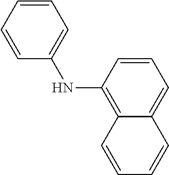 [SMILES: c1ccc(Nc2cccc3ccccc23)cc1] | 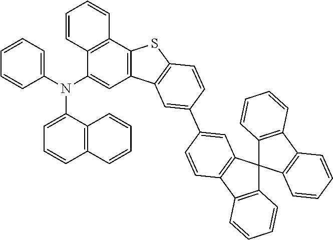 [SMILES: c1ccc(N(c2cccc3ccccc23)c2cc3c4cc(-c5ccc6c(c5)C5(c7ccccc7-c7ccccc75)c5ccccc5-6)ccc4sc3c3ccccc23)cc1] | 61% |
| 620 | 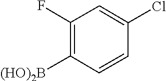 [SMILES: OB(O)c1ccc(Cl)cc1F] | 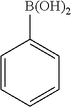 | 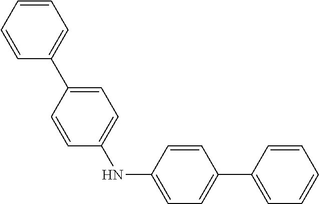 [SMILES: c1ccc(-c2ccc(Nc3ccc(-c4ccccc4)cc3)cc2)cc1] | 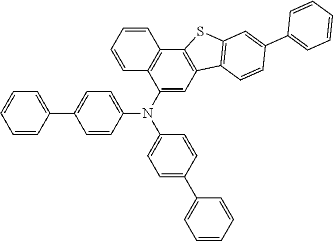 [SMILES: c1ccc(-c2ccc(N(c3ccc(-c4ccccc4)cc3)c3cc4c5ccc(-c6ccccc6)cc5sc4c4ccccc34)cc2)cc1] | 78% |
| 636 | 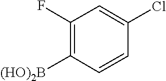 [SMILES: OB(O)c1ccc(Cl)cc1F] | 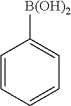 | 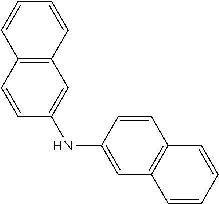 [SMILES: c1ccc2cc(Nc3ccc4ccccc4c3)ccc2c1] | 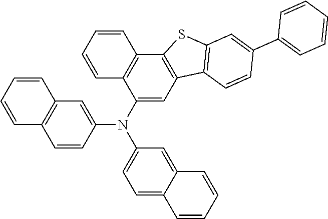 [SMILES: c1ccc(-c2ccc3c(c2)sc2c4ccccc4c(N(c4ccc5ccccc5c4)c4ccc5ccccc5c4)cc32)cc1] | 82% |
| 655 | 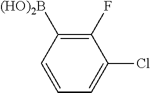 [SMILES: OB(O)c1cccc(Cl)c1F] | 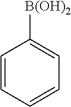 | 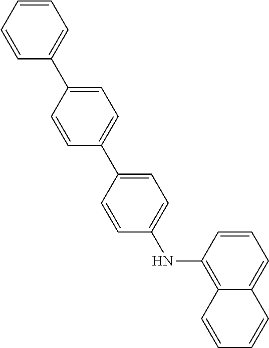 [SMILES: c1ccc(-c2ccc(-c3ccc(Nc4cccc5ccccc45)cc3)cc2)cc1] | 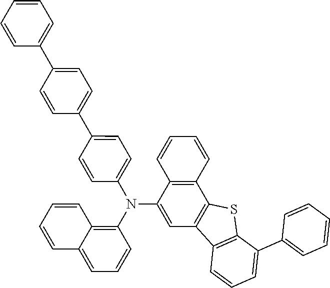 [SMILES: c1ccc(-c2ccc(-c3ccc(N(c4cccc5ccccc45)c4cc5c6cccc(-c7ccccc7)c6sc5c5ccccc45)cc3)cc2)cc1] | 65% |
| 660 | 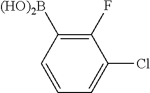 [SMILES: OB(O)c1cccc(Cl)c1F] | 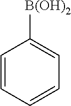 | 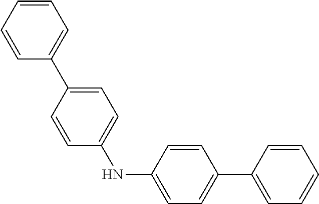 [SMILES: c1ccc(-c2ccc(Nc3ccc(-c4ccccc4)cc3)cc2)cc1] | 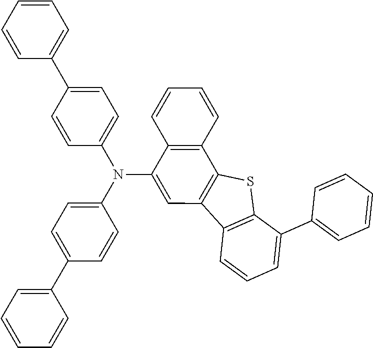 [SMILES: c1ccc(-c2ccc(N(c3ccc(-c4ccccc4)cc3)c3cc4c5cccc(-c6ccccc6)c5sc4c4ccccc34)cc2)cc1] | 85% |
| 673 | 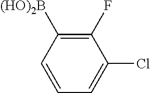 [SMILES: OB(O)c1cccc(Cl)c1F] | 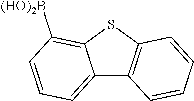 [SMILES: OB(O)c1cccc2c1sc1ccccc12] | 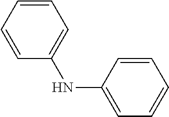 [SMILES: c1ccc(Nc2ccccc2)cc1] | 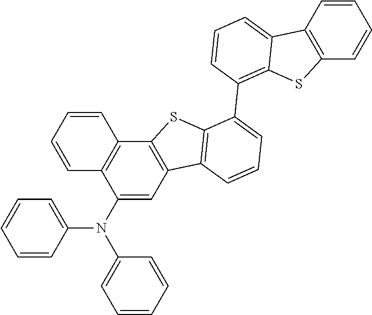 [SMILES: c1ccc(N(c2ccccc2)c2cc3c4cccc(-c5cccc6c5sc5ccccc56)c4sc3c3ccccc23)cc1] | 72% |
| 700 | 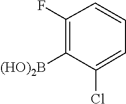 [SMILES: OB(O)c1c(F)cccc1Cl] | 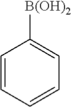 | 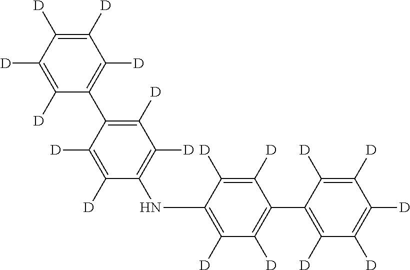 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 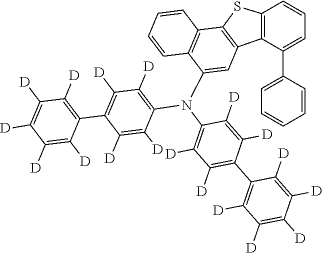 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4c(sc5cccc(-c6ccccc6)c54)c4ccccc34)c([2H])c2[2H])c([2H])c1[2H]] | 82% |

### Preparation Example 3. Preparation of Compound 689

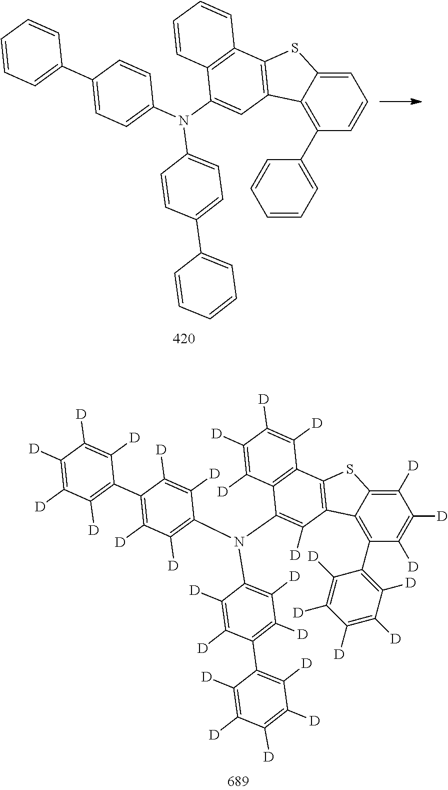 [SMILES: c1ccc(-c2ccc(N(c3ccc(-c4ccccc4)cc3)c3cc4c(sc5cccc(-c6ccccc6)c54)c4ccccc34)cc2)cc1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c4c(sc5c([2H])c([2H])c([2H])c(-c6c([2H])c([2H])c([2H])c([2H])c6[2H])c54)c4c([2H])c([2H])c([2H])c([2H])c34)c([2H])c2[2H])c([2H])c1[2H]]

Compound 420 (10.0 g, 15.1 mM) and trifluoromethanesulfonic acid (15.4 g, 102.7 mM) were dissolved in D₆ benzene (100 mL), and stirred for 1 hour at 60° C. After the reaction was terminated, the result was neutralized with an aqueous K₃PO₄ solution at roan temperature, and then extracted with dichloromethane and water (H₂O). The reaction material was purified by column chromatography (dichloromethane:hexane=1:1 volume ratio), and recrystallized with methanol to obtain Compound 689 (7.5 g, yield 71.5%).

### Preparation Example 4. Preparation of Compound 690

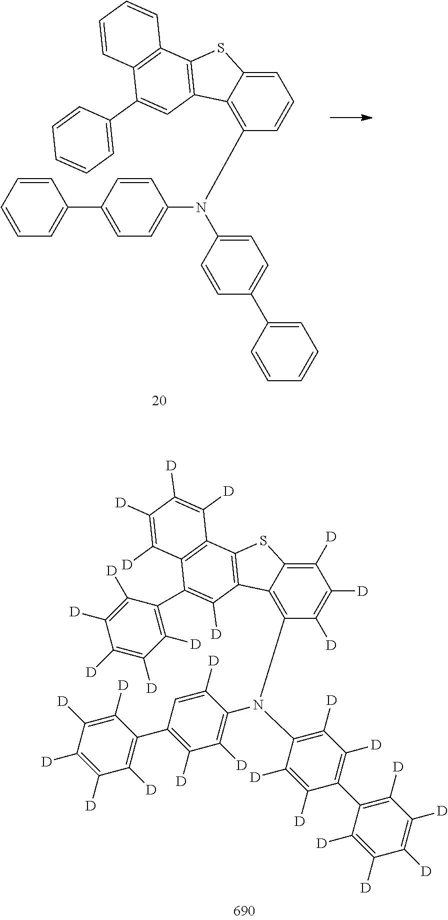 [SMILES: c1ccc(-c2ccc(N(c3ccc(-c4ccccc4)cc3)c3cccc4sc5c6ccccc6c(-c6ccccc6)cc5c34)cc2)cc1 ; [2H]c1cc(-c2c([2H])c([2H])c([2H])c([2H])c2[2H])c([2H])c([2H])c1N(c1c([2H])c([2H])c(-c2c([2H])c([2H])c([2H])c([2H])c2[2H])c([2H])c1[2H])c1c([2H])c([2H])c([2H])c2sc3c4c([2H])c([2H])c([2H])c([2H])c4c(-c4c([2H])c([2H])c([2H])c([2H])c4[2H])c([2H])c3c12]

Compound 20 (15.0 g, 24.4 mM) and trifluoromethanesulfonic acid (24.9 g, 165.9 mM) were dissolved in D₆ benzene (150 mL), and stirred for 1 hour at a temperature of 60° C. After the reaction was terminated, the result was neutralized with an aqueous K₃PO₄ solution at room temperature, and then extracted with dichloromethane and water (H₂O). The reaction material was purified by column chromatography (dichloramethane:hexane-1:1 volume ratio), and recrystallized with methanol to obtain Compound 690 (11.5 g, yield 73.0%).

Synthesis results for the compounds described in Preparation Example 1 to Preparation Example 4, and Table 1 and Table 2 are shown in the following Table 3 and Table 4. The following Table 3 shows measurement values of ¹H NMR (CDCl₃, 300 MHz), and the following Table 4 shows measurement values of FD-mass spectrometry (FD-MS: field desorption mass spectrometry).

TABLE 3

| Compound | |
|---|---|
| No. | ¹H NMR (CDCl₃, 300 MHz) |
| 20 | δ = 8.97(1H, d), 8.12(1H, d), 7.75~7.79(6H, m), 7.37~7.59(22H, |
| | m) |
| 24 | δ = 8.97(1H, d), 8.10~8.12(2H, t), 7. 75~7.79(4H, m), 7.67 (1H, |
| | s), 7.37~7.55(11H, m), 7.25(4H, s), 7.08(2H, m) |
| 32 | δ = 8.97(1H, d), 8.12(1H, d), 7.75~7.90(8H, m), 7.67 (1H, |
| | s), 7.19~7.55(25H, m), 7.06(1H, s) |
| 40 | δ = 8.97(1H, d), 8.10~8.12(2H, t), 7.98(1H, s), 7.79(2H, t), |
| | 7.28~7.59(19H, m), 6.98~7.14(3H, m), 6.97 (1H, s) |
| 48 | δ = 8.97(1H, t), 8.5(1H, d), 8.09~8.20(3H, m), 7.75~7.77(3H, |
| | m), 7.39~7.59(12H, m), 7.17~7.24(8H, m) |
| 56 | δ = 8.97(1H, t), 8.5(1H, d), 8.20(1H, d), 8.09~8.12(3H, t), |
| | 7.75~7.77(3H, t), 7.08(1H, s), 7.39~7.59(12H, m), |
| | 7.25~7.45(10H, m) |
| 64 | δ = 8.97(1H, t), 8.5(1H, d), 8.20(1H, d), 8.09~8.12(2H, t), |
| | 7.55~7.75(5H, m), 7.67 (1H, s), 7.37~7.55(21H, m), 7.25(4H, |
| | s), |
| 71 | δ = 8.97(1H, d), 7.98~8.15(6H, m), 7.81(1H, s), 7.08~7.64(20H, |
| | m), 7.00~7.08(3H, m) |
| 72 | δ = 8.97(1H, d), 7.98~8.12(4H, m), 7.67~7.78(5H, m), |
| | 7.31~7.59(21H, m), 7.11(1H, s) |
| 80 | δ = 8.97(1H, d), 8.55(2H, d), 8.32(2H, d), 8.12(1H, d), |
| | 7.67~7.78(7H, m), 7.41~7.59(18H, m), 7.11(2H, s) |
| 96 | δ = 8.97(1H, d), 8.10~8.12(2H, t), 7.89~7.90(2H, t), |
| | 7.27~7.59(26H, m), 7.11(2H, s) |
| 98 | δ = 8.97(1H, d), 8.12 (1H, d), 8.04 (3H, s), 7.75(6H, d), |
| | 7.37~7.59(15H, m), 7.24~7.25(6H, m), 7.00~7.08 (3H, m) |
| 120 | δ = 8.97(1H, d), 8.12 (1H, d), 7.95 (1H, s), 7.67 (1H, s), |
| | 7.37~7.59(20H, m) |
| 138 | δ = 8.97(1H, d), 8.12 (1H, d), 7.95~7.98(2H, m), |
| | 7.28~7.59(25H, m), 6.97(1H, d) |
| 141 | δ = 8.97(1H, d), 7.95~8.12(5H, m), 7.75~7.85(5H, m), 7.67 (1H, |
| | s), 7.27~7.59 (19H, m), 7.17~7.18(2H, m) |
| 179 | δ = 8.97(1H, d), 8.55 (2H, d), 8.32 (2H, d), 8.09~8.12(3H, m), |
| | 7.95 (1H, s), 7.81~7.85(2H, m), 7.41~7.63(10H, m), 7.24(2H, |
| | t), 7.08(3H, m) |
| 191 | δ = 8.97(1H, d), 8.09~8.15(4H, m), 7.78~7.90 (6H, m) |
| | 7.38~7.63(10H, m), 7.08~7.26(15H, m) |
| 220 | δ = 8.97(1H, d), 8.12 (1H, d), 8.01 (1H, d), 7.7~7.59 (28H, |
| | m) |
| 228 | δ = 8.97(1H, d), 8.10~8.12(2H, m), 8.07 (1H, d), 7.79~7.90 |
| | (4H, m), 7.33~7.46 (12H, m), 7.08~7.16 (4H, m), 1.69(6H, s) |
| 236 | δ = 8.97(1H, d), 8.12 (1H, d), 8.01 (1H, d), 7.79~7.90 (6H, |
| | m), 7.28~7.46 (10H, m), 7.16(2H, d), 1.69(12H, s) |
| 243 | δ = 8.97(2H, t), 8.50 (1H, d), 8.09~8.20(3H, m), 8.01 (1H, |
| | d), 7.77(1H, t), 7.52~7.67 (5H, m), 7.39~7.43 (2H, m), |
| | 7.24(4H, t), 7.00~7.08 (6H, m) |
| 251 | δ = 8.97(2H, t), 8.50 (1H, d), 8.01~8.20(7H, m), 7.77~7.81 |
| | (2H, m), 7.37~7.63 (16H, m), 7.08~7.14 (3H, m) |
| 259 | δ = 8.97(2H, t), 8.50 (1H, d), 8.09~8.20(3H, m), 8.01 (1H, d), |
| | 7.39~7.68 (23H, m), 7.25(4H, s), 7.11(1H, s) |
| 292 | δ = 8.97(1H, d), 8.09~8.12(2H, m), 8.01 (1H, d), 7.89~7.90 |
| | (2H, m), 7.38~7.78 (20H, m), 7.10~7.28 (12H, m) |
| 332 | δ = 8.97(1H, d), 8.11~8.12(2H, t), 7.75~7.79 (8H, m), 7.67 |
| | (1H, s), 7.17~7.59 (13H, m), 7.06 (1H, d) |
| 377 | δ = 8.97(1H, d), 8.55(1H, d), 8.32(1H, d), 8.11~8.12(2H, t), |
| | 7.67~7.70 (3H, m), 7.41~7.59 (9H, m), 7.24 (4H, t), |
| | 7.00~7.08 (6H, m) |
| 684 | δ = 8.97(1H, d), 8.12(1H, d), 7.75 (4H, d), 7.37~7.59 (20H, |
| | m) |
| 688 | δ = 8.97(1H, d), 8.12(1H, d), 7.75 (2H, d), 7.67 (1H, s), |
| | 7.41~7.59 (8H, m), 7.25 (4H, s) |
| 693 | δ = 8.97(1H, d), 8.12(1H, d), 7.75 (4H, d), 7.37~7.59 (20H, |
| | m) |
| 420 | δ = 8.12(2H, d), 8.03(1H, d), 7.94~7.95 (2H, t), 7.37~7.79 |
| | (25H, m) |
| 432 | δ = 8.12(2H, d), 8.03(1H, d), 7.19~7.90 (21H, m), 7.06 (1H, |
| | d) |
| 440 | δ = 7.94~8.12 (7H, m), 7.79 (2H, d), 7.28~7.59 (15H, m), |
| | 7.08~7.14 (3H, m), 6.97 (1H, d) |
| 447 | δ = 8.95(1H, d), 8.50(1H, d), 8.03~8.12(5H, m), 7.94~7.95 (2H, |
| | t), 7.37~7.77 (15H, m), 7.24 (2H, t), 7.00~7.08 (3H, m) |
| 455 | δ = 8.95(1H, d), 8.50(1H, d), 8.20(1H, d), 8.03~8.12(4H, m), |
| | 7.94~7.95 (2H, t), 7.41~7.77 (12H, m), 7.17~7.25 (9H, m), |
| | 7.00~7.08 (3H, m) |
| 467 | δ = 7.94~8.12 (10H, m), 7.81 (1H, t), 7.39~7.63 (13H, m), |
| | 7.00~7.08 (3H, m) |
| 475 | δ = 8.55(1H, d), 8.45(1H, d), 8.32(1H, d), 8.12~8.22(3H, m), |
| | 8.03(1H, d), 7.93~7.95 (3H, t), 7.81(1H, d), 7.49~7.70 (10H, |
| | m), 7.24 (2H, t), 7.00~7.08 (3H, m) |
| 487 | δ = 8.45(1H, d), 8.03~8.13(7H, m), 7.93~7.95 (3H, t), |
| | 7.79~7.81 (3H, m), 7.41~7.59 (12H, m), 7.24 (2H, t), |
| | 7.00~7.08 (3H, m) |
| 495 | δ = 8.03~8.15(6H, m), 7.81~7.95 (7H, m), 7.24~7.59 (17H, m), |
| | 7.00~7.08 (3H, m) |
| 507 | δ = 8.10~8.12(6H, m), 7.95~7.99 (2H, m), 7.75 (2H, d), |
| | 7.39~7.59 (10H, m), 7.24 (2H, t), 7.00~7.08 (5H, m) |
| 515 | δ = 8.12~8.15(6H, m), 7.95~7.99 (2H, m), 7.75~7.81 (5H, m), |
| | 7.39~7.63 (13H, m), 7.25 (4H, s) |
| 531 | δ = 8.12(4H, d), 7.85~7.99 (4H, m), 7.24 (4H, t), 7.27~7.59 |
| | (20H, m), 7.06 (1H, s) |
| 543 | δ = 8.95(1H, d), 8.50(1H, d), 8.09~8.20(6H, m), 7.77(1H, t), |
| | 7.50~7.59 (3H, m), 7.39(1H, t), 7.24 (4H, t), 7.00~7.08 (6H, |
| | m) |
| 555 | δ = 8.95(1H, d), 8.50(1H, d), 8.09~8.20(6H, m), 7.95~7.99 (3H, |
| | t), 7.75~7.77 (3H, t), 7.39~7.59 (6H, m), 7.17~7.27 (9H, m), |
| | 7.00~7.08 (3H, m) |
| 565 | δ = 7.98~8.12(9H, m), 7.50~7.59 (4H, t), 7.24~7.39 (6H, m), |
| | 7.00~7.08 (6H, m) |
| 579 | δ = 8.55(2H, d), 8.32(2H, d), 8.12~8.22(6H, m), 7.95~7.99 (3H, |
| | t), 7.81(1H, t), 7.41~7.70 (13H, m), 7.24(2H, t), 7.00~7.08 |
| | (3H, m) |
| 595 | δ = 8.08~8.22(7H, m), 7.79~7.99 (7H, m), 7.24~7.68 (17H, m), |
| | 7.00~7.08 (3H, m) |
| 620 | δ = 8.12~8.24(5H, m), 7.95(1H, s), 7.75(6H, t), 7.37~7.55 |
| | (19H, m) |
| 636 | δ = 8.55(1H, d), 8.45(1H, d), 8.32(1H, d), 8.12~8.24(5H, m), |
| | 7.93~7.95 (2H, t), 7.70~7.78 (5H, m), 7.40~7.59 (12H, m), |
| | 7.11(2H, s) |
| 655 | δ = 8.55(1H, d), 8.32(1H, d), 8.12~8.22(4H, m), 7.95(1H, s), |
| | 7.41~7.75 (22H, m), 7.25(4H, s) |
| 660 | δ = 8.55(1H, d), 8.32(1H, d), 8.12(2H, d), 7.95(1H, s), |
| | 7.70~7.75 (5H, m), 7.37~7.59 (10H, m) |
| 673 | δ = 8.55(2H, d), 8.45(1H, d), 8.32(2H, d), 8.12(2H, d), |
| | 7.70(2H, t), 7.49~7.59 (4H, m), 7.24(4H, t), 7.00~7.08 (6H, |
| | m) |
| 700 | δ = 7.94~8.12(5H, m), 7.79(2H, d), 7.41~7.68(6H, m), |
| 689 | Not detected since there is no proton(H) |
| 690 | Not detected since there is no proton(H) |

TABLE 4

| Compound | FD-MS | Compound | FD-MS |
|---|---|---|---|
| 20 | Molecular Weight: 629.82 | 688 | Molecular Weight: 724.03 |
| 24 | Molecular Weight: 705.92 | 693 | Molecular Weight: 726.94 |
| 32 | Molecular Weight: 792.01 | 420 | Molecular Weight: 629.82 |
| 40 | Molecular Weight: 643.80 | 432 | Molecular Weight: 792.01 |
| 48 | Molecular Weight: 603.78 | 440 | Molecular Weight: 643.80 |
| 56 | Molecular Weight: 679.88 | 447 | Molecular Weight: 603.78 |
| 64 | Molecular Weight: 755.98 | 455 | Molecular Weight: 679.88 |
| 71 | Molecular Weight: 693.86 | 467 | Molecular Weight: 617.77 |
| 72 | Molecular Weight: 743.92 | 475 | Molecular Weight: 633.83 |
| 80 | Molecular Weight: 759.98 | 487 | Molecular Weight: 709.92 |
| 96 | Molecular Weight: 816.03 | 495 | Molecular Weight: 765.97 |
| 98 | Molecular Weight: 782.02 | 507 | Molecular Weight: 553.72 |
| 120 | Molecular Weight: 629.82 | 515 | Molecular Weight: 679.88 |
| 138 | Molecular Weight: 643.80 | 531 | Molecular Weight: 792.01 |
| 141 | Molecular Weight: 719.90 | 543 | Molecular Weight: 527.68 |
| 179 | Molecular Weight: 709.92 | 555 | Molecular Weight: 679.88 |
| 191 | Molecular Weight: 767.99 | 565 | Molecular Weight: 567.71 |
| 220 | Molecular Weight: 629.82 | 579 | Molecular Weight: 709.92 |
| 228 | Molecular Weight: 669.89 | 595 | Molecular Weight: 765.97 |
| 236 | Molecular Weight: 709.95 | 620 | Molecular Weight: 629.82 |
| 243 | Molecular Weight: 527.68 | 636 | Molecular Weight: 683.89 |
| 251 | Molecular Weight: 653.84 | 655 | Molecular Weight: 679.88 |
| 259 | Molecular Weight: 729.94 | 660 | Molecular Weight: 629.82 |
| 292 | Molecular Weight: 818.05 | 673 | Molecular Weight: 583.77 |
| 332 | Molecular Weight: 792.01 | 700 | Molecular Weight: 647.93 |
| 377 | Molecular Weight: 659.86 | 689 | Molecular Weight: 661.01 |
| 684 | Molecular Weight: 714.97 | 690 | Molecular Weight: 661.01 |

### Experimental Example

### Experimental Example 1

### Experimental Example 1-1. Manufacture of Organic Light Emitting Device

A transparent electrode ITO thin film obtained from glass for an OLED (manufactured by Samsung-Corning Co., Ltd.) was ultrasonic cleaned for 5 minutes using trichloroethylene, acetone, ethanol and distilled water consecutively, stored in isopropanol, and used.

To a cell in a vacuum deposition apparatus, 4,4′,4″-tris(N,N-(2-naphthyl)-phenylamino)triphenyl amine (2-TNATA) was introduced.

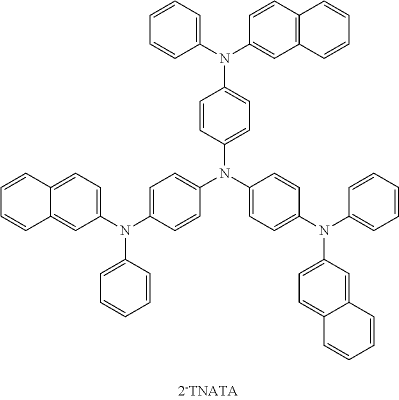 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate. To another cell in the vacuum deposition apparatus, the compound represented by Chemical Formula 1 or the following comparative compound described in the following Table 5 was introduced, and evaporated by applying a current to the cell to deposit a hole transport layer having a thickness of 300 Å on the hole injection layer.

After forming the hole injection layer and the hole transport layer as above, a blue light emitting material having the following structure was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

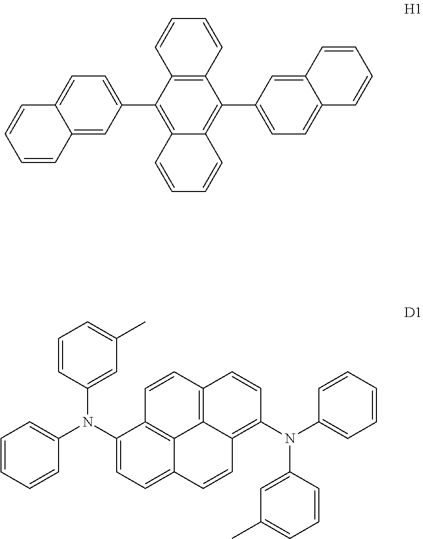

After that, a compound of the following Structural Formula E1 was deposited to a thickness of 300 Å as an electron transport layer.

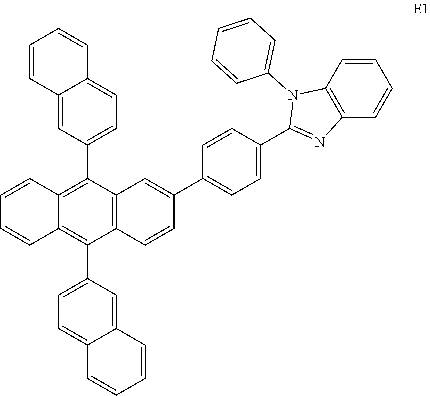 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

After that, lithium fluoride (LiF) was deposited to a thickness of 10 Å as an electron injection layer, and a negative electrode was formed by depositing aluminum (Al) to a thickness of 1,000 Å on the electron injection layer, and as a result, an organic light emitting device was manufactured. Meanwhile, all the organic compounds required to manufacture the organic light emitting device were vacuum sublimation purified under 10⁻⁸ torr to 10⁻⁶ torr by each material to be used in the organic light emitting device manufacture.

Herein, the comparative compounds used in the hole transport layer of the comparative examples are as follows.

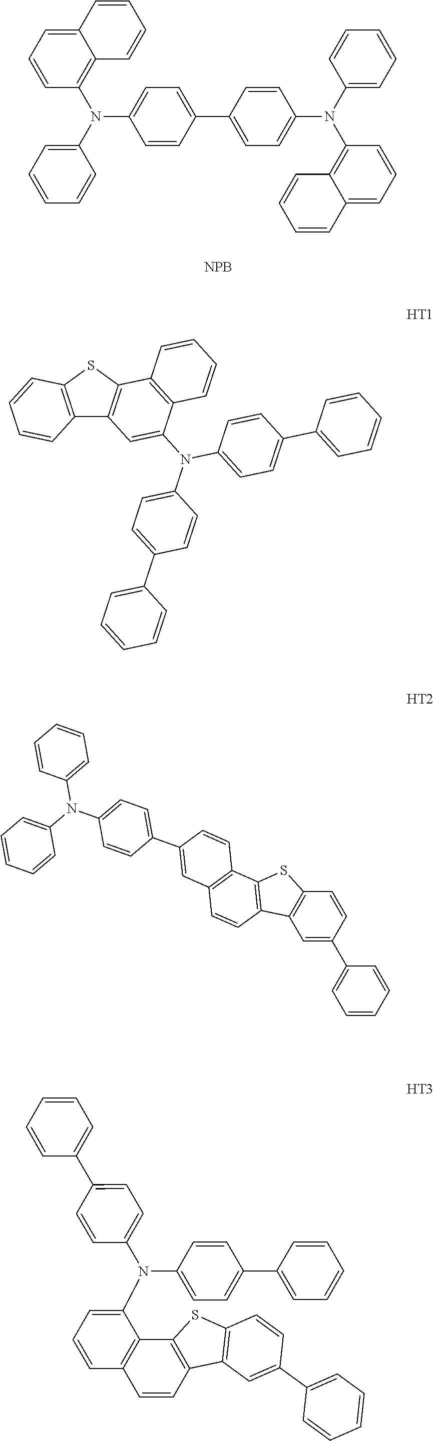 [SMILES: c1ccc(N(c2ccc(-c3ccc(N(c4ccccc4)c4cccc5ccccc45)cc3)cc2)c2cccc3ccccc23)cc1 ; "NPB" ; c1ccc(-c2ccc(N(c3ccc(-c4ccccc4)cc3)c3cc4c5ccccc5sc4c4ccccc34)cc2)cc1 ; c1ccc(-c2ccc3sc4c5ccc(-c6ccc(N(c7ccccc7)c7ccccc7)cc6)cc5ccc4c3c2)cc1 ; c1ccc(-c2ccc(N(c3ccc(-c4ccccc4)cc3)c3cccc4ccc5c6cc(-c7ccccc7)ccc6sc5c34)cc2)cc1]

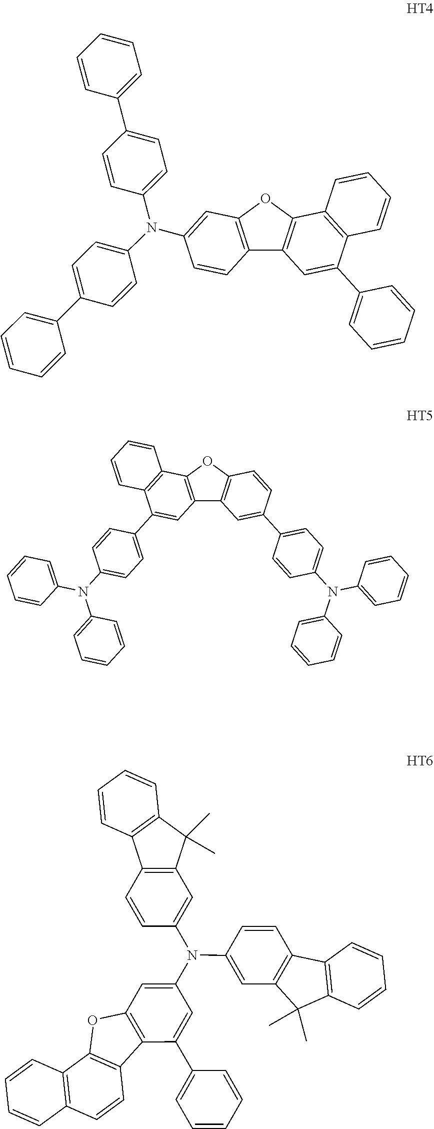 [SMILES: c1ccc(-c2ccc(N(c3ccc(-c4ccccc4)cc3)c3ccc4c(c3)oc3c5ccccc5c(-c5ccccc5)cc43)cc2)cc1 ; c1ccc(N(c2ccccc2)c2ccc(-c3ccc4oc5c6ccccc6c(-c6ccc(N(c7ccccc7)c7ccccc7)cc6)cc5c4c3)cc2)cc1 ; CC1(C)c2ccccc2-c2ccc(N(c3ccc4c(c3)C(C)(C)c3ccccc3-4)c3cc(-c4ccccc4)c4c(c3)oc3c5ccccc5ccc34)cc21]

### Experimental Example 1-2. Driving Voltage and Light Emission Efficiency of Organic Light Emitting Device

For each of the organic electroluminescent devices manufactured as above, electroluminescent (EL) properties were measured using M7000 manufactured by McScience Inc., and with the measurement results, a lifetime T9₅ that is a time taken to became 95% with respect to initial luminance was measured when standard luminance was 700 cd/m² through a lifetime measurement system (M6000) manufactured by McScience Inc.

Results of measuring driving voltage, light emission efficiency, color coordinate (CIE) and lifetime (T9₅) of the blue organic light emitting devices manufactured according to the above-described manufacturing method are as shown in Table 5.

TABLE 5

| | | | Light | | |
|---|---|---|---|---|---|
| | | Driving | Emission | | |
| | | Voltage | Efficiency | | Lifetime |
| | Compound | (V) | (cd/A) | CIE (x, y) | (T₉₅) |
| Example 1 | 20 | 4.80 | 6.60 | (0.134, 0.100) | 53 |
| Example 2 | 24 | 4.78 | 6.64 | (0.134, 0.100) | 49 |
| Example 3 | 32 | 4.75 | 6.69 | (0.134, 0.100) | 47 |
| Example 4 | 40 | 4.73 | 6.70 | (0.134, 0.101) | 48 |
| Example 5 | 48 | 4.79 | 6.68 | (0.134, 0.101) | 50 |
| Example 6 | 56 | 4.78 | 6.73 | (0.134, 0.100) | 49 |
| Example 7 | 64 | 4.81 | 6.70 | (0.134, 0.100) | 49 |
| Example 8 | 71 | 4.84 | 6.68 | (0.134, 0.100) | 46 |
| Example 9 | 72 | 4.79 | 6.60 | (0.134, 0.100) | 47 |
| Example 10 | 80 | 4.76 | 6.81 | (0.134, 0.101) | 49 |
| Example 11 | 96 | 4.88 | 6.74 | (0.134, 0.101) | 50 |
| Example 12 | 98 | 4.83 | 6.70 | (0.134, 0.100) | 46 |
| Example 13 | 120 | 4.75 | 6.69 | (0.134, 0.100) | 44 |
| Example 14 | 138 | 4.69 | 6.68 | (0.134, 0.101) | 49 |
| Example 15 | 141 | 4.61 | 6.83 | (0.134, 0.101) | 50 |
| Example 16 | 179 | 4.80 | 6.60 | (0.134, 0.100) | 53 |
| Example 17 | 191 | 4.78 | 6.64 | (0.134, 0.100) | 49 |
| Example 18 | 220 | 4.75 | 6.69 | (0.134, 0.100) | 47 |
| Example 19 | 228 | 4.73 | 6.70 | (0.134, 0.101) | 48 |
| Example 20 | 236 | 4.79 | 6.68 | (0.134, 0.101) | 50 |
| Example 21 | 243 | 4.78 | 6.73 | (0.134, 0.100) | 49 |
| Example 22 | 251 | 4.81 | 6.70 | (0.134, 0.100) | 49 |
| Example 23 | 259 | 4.84 | 6.68 | (0.134, 0.100) | 46 |
| Example 24 | 292 | 4.79 | 6.60 | (0.134, 0.100) | 47 |
| Example 25 | 332 | 4.76 | 6.81 | (0.134, 0.101) | 49 |
| Example 26 | 377 | 4.88 | 6.74 | (0.134, 0.101) | 50 |
| Example 27 | 684 | 4.83 | 6.70 | (0.134, 0.100) | 46 |
| Example 28 | 688 | 4.75 | 6.69 | (0.134, 0.100) | 44 |
| Example 29 | 693 | 4.69 | 6.68 | (0.134, 0.101) | 49 |
| Example 30 | 420 | 4.61 | 6.83 | (0.134, 0.101) | 50 |
| Example 31 | 432 | 4.80 | 6.60 | (0.134, 0.100) | 53 |
| Example 32 | 440 | 4.78 | 6.64 | (0.134, 0.100) | 49 |
| Example 33 | 447 | 4.75 | 6.69 | (0.134, 0.100) | 47 |
| Example 34 | 455 | 4.73 | 6.70 | (0.134, 0.101) | 48 |
| Example 35 | 467 | 4.79 | 6.68 | (0.134, 0.101) | 50 |
| Example 36 | 475 | 4.78 | 6.73 | (0.134, 0.100) | 49 |
| Example 37 | 487 | 4.81 | 6.70 | (0.134, 0.100) | 49 |
| Example 38 | 495 | 4.84 | 6.68 | (0.134, 0.100) | 46 |
| Example 39 | 507 | 4.79 | 6.60 | (0.134, 0.100) | 47 |
| Example 40 | 515 | 4.76 | 6.81 | (0.134, 0.101) | 49 |
| Example 41 | 531 | 4.88 | 6.74 | (0.134, 0.101) | 50 |
| Example 42 | 543 | 4.83 | 6.70 | (0.134, 0.100) | 46 |
| Example 43 | 555 | 4.75 | 6.69 | (0.134, 0.100) | 44 |
| Example 44 | 565 | 4.69 | 6.68 | (0.134, 0.101) | 49 |
| Example 45 | 579 | 4.61 | 6.83 | (0.134, 0.101) | 50 |
| Example 46 | 595 | 4.81 | 6.70 | (0.134, 0.100) | 49 |
| Example 47 | 620 | 4.84 | 6.68 | (0.134, 0.100) | 46 |
| Example 48 | 636 | 4.79 | 6.60 | (0.134, 0.100) | 47 |
| Example 49 | 655 | 4.76 | 6.81 | (0.134, 0.101) | 49 |
| Example 50 | 660 | 4.88 | 6.74 | (0.134, 0.101) | 50 |
| Example 51 | 673 | 4.83 | 6.70 | (0.134, 0.100) | 46 |
| Example 52 | 700 | 4.75 | 6.69 | (0.134, 0.100) | 44 |
| Example 53 | 689 | 4.69 | 6.68 | (0.134, 0.101) | 49 |
| Example 54 | 690 | 4.61 | 6.83 | (0.134, 0.101) | 50 |
| Comparative | NPB | 5.16 | 6.10 | (0.134, 0.101) | 32 |
| Example 1 | | | | | |
| Comparative | HT1 | 5.19 | 6.11 | (0.134, 0.101) | 33 |
| Example 2 | | | | | |
| Comparative | HT2 | 5.20 | 6.19 | (0.134, 0.101) | 31 |
| Example 3 | | | | | |
| Comparative | HT3 | 5.20 | 6.22 | (0.134, 0.101) | 35 |
| Example 4 | | | | | |
| Comparative | HT4 | 5.20 | 6.22 | (0.134, 0.101) | 38 |
| Example 5 | | | | | |
| Comparative | HT5 | 5.20 | 6.11 | (0.134, 0.101) | 35 |
| Example 6 | | | | | |
| Comparative | HT6 | 5.20 | 6.22 | (0.134, 0.101) | 38 |
| Example 7 | | | | | |

From the results of Table 5, it was identified that the organic light emitting device using the hole transport layer material comprising the heterocyclic compound of the present disclosure had lower driving voltage and significantly improved light emission efficiency and lifetime compared to the organic light emitting device of the comparative example.

Specifically, in the heterocyclic compound of the present disclosure, the unshared electron pair of amine enables excellent hole flow, and enhances a hole transport ability of the hole transport layer. In addition, it was identified that, as Ar1 and Ar2 of Chemical Formula 2 having strengthened hole properties and the amine moiety bonded, planarity and glass transition temperature of the amine derivative increased, which increased thermal stability of the heterocyclic compound of the present disclosure. In addition, it was identified that the organic light emitting device had superior light emission efficiency and lifetime by Ar1 and Ar2 delocalizing the energy level of the heterocyclic compound and thereby stabilizing the HOMO energy.

Moreover, it was identified that, through adjusting the band gap and the T1 value (energy level value in triplet state), hole transport ability was enhanced and molecular stability increased, and as a result, the organic light emitting device had lowered driving voltage and enhanced light emission efficiency, and the organic light emitting device had enhanced lifetime properties by thermal stability of the compound.

The hole properties refer to properties capable of forming holes by donating electrons when applying an electric field, and mean properties facilitating injection of holes formed in a positive electrode to a light emitting layer, migration of holes formed in a light emitting layer to a positive electrode and migration in a light emitting layer by having conductive properties along the HOMO level. Electron properties refer to properties capable of receiving electrons when applying an electric field, and mean properties facilitating injection of electrons formed in a negative electrode to a light emitting layer, migration of electrons formed in a light emitting layer to a negative electrode and migration in a light emitting layer by having conductive properties along the LUMO level.

### Experimental Example 2

### Experimental Example 2-1. Manufacture of Organic Light Emitting Device

A glass substrate on which ITO (indium tin oxide) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function increase and residual film removal, the substrate was transferred to a thermal deposition apparatus for organic deposition.

Subsequently, the chamber was evacuated until the degree of vacuum therein reached 10⁻⁶ torr, and then 2-TNATA was evaporated by applying a current to the cell to deposit a hole injection layer on the ITO substrate to a thickness of 600 Å.

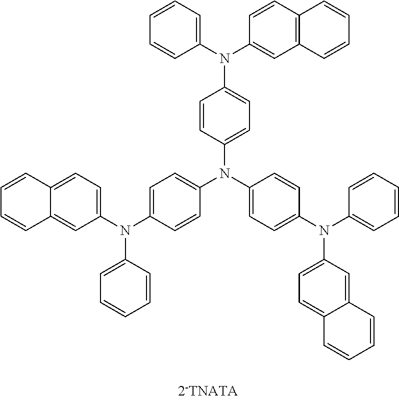 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

To another cell in the vacuum deposition apparatus, the following N,N′-bis(α-naphthyl)-N,N′-diphenyl-4,4′-diamine (NPB) was introduced, and evaporated by applying a current to the cell to deposit a hole transport layer having a thickness of 300 Å on the hole injection layer.

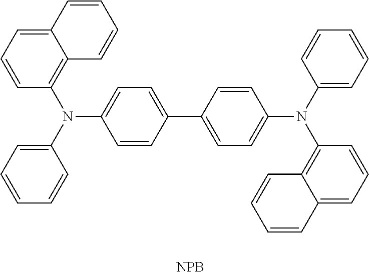 [SMILES: c1ccc(N(c2ccc(-c3ccc(N(c4ccccc4)c4cccc5ccccc45)cc3)cc2)c2cccc3ccccc23)cc1 ; "NPB"]

After that, the compound represented by Chemical Formula 1 or the following comparative compound described in the following Table 6 was deposited to a thickness of 100 Å as a light emitting auxiliary layer.

A light emitting layer was thermal vacuum deposited thereon as follows. As the light emitting layer, a compound of 9-[4-(4,6-diphenyl-1,3,5-triazin-2-yl)phenyl]-9′-phenyl-3,3′-bi-9H-carbazole was deposited to 400 Å as a host, and Ir(ppy)₃ was doped by 7% and deposited as a green phosphorescent dopant. After that, BCP was deposited to 60 Å as a hole blocking layer, and Alq₃ was deposited to 200 Å thereon as an electron transport layer.

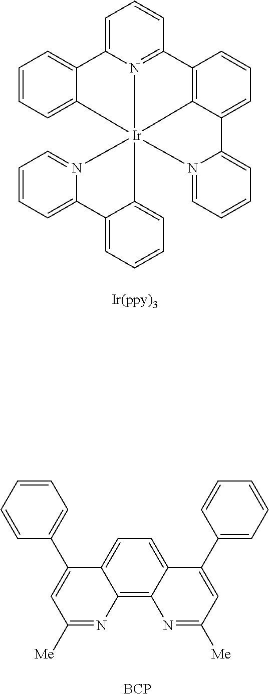

Lastly, an electron injection layer was formed on the electron transport layer by depositing lithium fluoride (LiF) to a thickness of 10 Å, and then a negative electrode was formed on the electron injection layer by depositing aluminum (Al) to a thickness of 1,200 Å, and as a result, an organic light emitting device was manufactured.

Meanwhile, all the organic compounds required to manufacture the organic light emitting device were vacuum sublimation purified under 10⁻⁸ torr to 10⁻⁶ torr by each material to be used in the organic light emitting device manufacture.

Herein, the comparative compounds used in the light emitting auxiliary layer of the comparative examples are as follows.

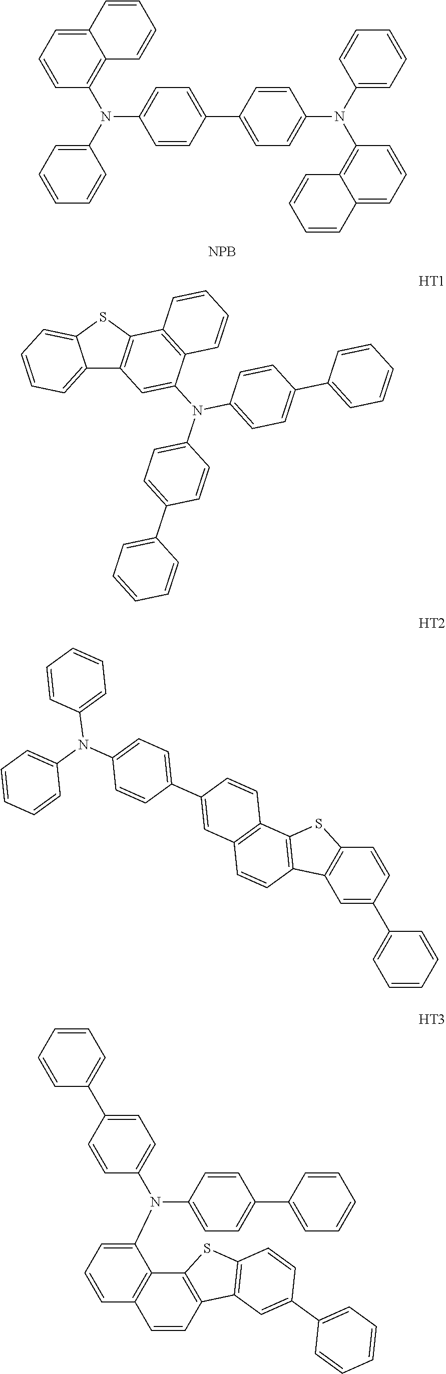 [SMILES: c1ccc(N(c2ccc(-c3ccc(N(c4ccccc4)c4cccc5ccccc45)cc3)cc2)c2cccc3ccccc23)cc1 ; "NPB" ; c1ccc(-c2ccc(N(c3ccc(-c4ccccc4)cc3)c3cc4c5ccccc5sc4c4ccccc34)cc2)cc1 ; c1ccc(-c2ccc3sc4c5ccc(-c6ccc(N(c7ccccc7)c7ccccc7)cc6)cc5ccc4c3c2)cc1 ; c1ccc(-c2ccc(N(c3ccc(-c4ccccc4)cc3)c3cccc4ccc5c6cc(-c7ccccc7)ccc6sc5c34)cc2)cc1]

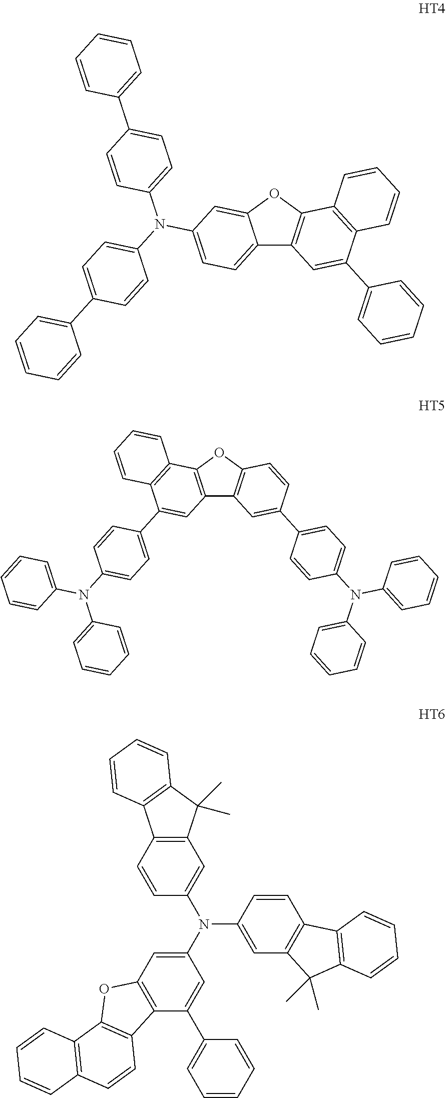 [SMILES: c1ccc(-c2ccc(N(c3ccc(-c4ccccc4)cc3)c3ccc4c(c3)oc3c5ccccc5c(-c5ccccc5)cc43)cc2)cc1 ; c1ccc(N(c2ccccc2)c2ccc(-c3ccc4oc5c6ccccc6c(-c6ccc(N(c7ccccc7)c7ccccc7)cc6)cc5c4c3)cc2)cc1 ; CC1(C)c2ccccc2-c2ccc(N(c3ccc4c(c3)C(C)(C)c3ccccc3-4)c3cc(-c4ccccc4)c4c(c3)oc3c5ccccc5ccc34)cc21]

### Experimental Example 2-2. Driving Voltage and Light Emission Efficiency of Organic Light Emitting Device

For each of the organic electroluminescent devices manufactured as above, electroluminescent (EL) properties were measured using M7000 manufactured by McScience Inc., and with the measurement results, a lifetime T₉₀ that is a time taken to become 90% with respect to initial luminance was measured when standard luminance was 6,000 cd/m² through a lifetime measurement system (M6000) manufactured by McScience Inc.

Results of measuring driving voltage, light emission efficiency and lifetime (T₉₀) of the green organic light emitting devices manufactured according to the above-described manufacturing method are as shown in Table 6.

TABLE 6

| | | Driving | Light Emission | |
|---|---|---|---|---|
| | | Voltage | Efficiency | Lifetime |
| | Compound | (V) | (cd/A) | (T₉₀) |
| Example 55 | 20 | 4.73 | 6.68 | 208 |
| Example 56 | 24 | 4.80 | 6.74 | 220 |
| Example 57 | 32 | 4.77 | 6.66 | 253 |
| Example 58 | 40 | 4.76 | 6.63 | 213 |
| Example 59 | 48 | 4.76 | 6.72 | 213 |
| Example 60 | 56 | 4.73 | 6.80 | 197 |
| Example 61 | 64 | 4.79 | 6.73 | 220 |
| Example 62 | 71 | 4.70 | 6.70 | 210 |
| Example 63 | 72 | 4.76 | 6.69 | 240 |
| Example 64 | 80 | 4.73 | 6.71 | 256 |
| Example 65 | 96 | 4.80 | 6.73 | 224 |
| Example 66 | 98 | 4.73 | 6.69 | 213 |
| Example 67 | 120 | 4.81 | 6.75 | 242 |
| Example 68 | 138 | 4.79 | 6.73 | 222 |
| Example 69 | 141 | 4.76 | 6.63 | 213 |
| Example 70 | 179 | 4.76 | 6.72 | 213 |
| Example 71 | 191 | 4.75 | 6.70 | 250 |
| Example 72 | 220 | 4.80 | 6.74 | 220 |
| Example 73 | 228 | 4.77 | 6.66 | 253 |
| Example 74 | 236 | 4.76 | 6.63 | 213 |
| Example 75 | 243 | 4.76 | 6.72 | 213 |
| Example 76 | 251 | 4.73 | 6.80 | 197 |
| Example 77 | 259 | 4.79 | 6.73 | 220 |
| Example 78 | 292 | 4.70 | 6.70 | 210 |
| Example 79 | 332 | 4.76 | 6.69 | 240 |
| Example 80 | 377 | 4.73 | 6.71 | 256 |
| Example 81 | 684 | 4.80 | 6.73 | 224 |
| Example 82 | 688 | 4.73 | 6.69 | 213 |
| Example 83 | 693 | 4.81 | 6.75 | 242 |
| Example 84 | 420 | 4.79 | 6.73 | 222 |
| Example 85 | 432 | 4.76 | 6.63 | 213 |
| Example 86 | 440 | 4.76 | 6.72 | 213 |
| Example 87 | 447 | 4.73 | 6.80 | 197 |
| Example 88 | 455 | 4.79 | 6.73 | 220 |
| Example 89 | 467 | 4.70 | 6.70 | 210 |
| Example 90 | 475 | 4.76 | 6.69 | 240 |
| Example 91 | 487 | 4.73 | 6.71 | 256 |
| Example 92 | 495 | 4.80 | 6.73 | 224 |
| Example 93 | 507 | 4.73 | 6.69 | 213 |
| Example 94 | 515 | 4.81 | 6.75 | 242 |
| Example 95 | 531 | 4.79 | 6.73 | 222 |
| Example 96 | 543 | 4.76 | 6.63 | 213 |
| Example 97 | 555 | 4.76 | 6.72 | 213 |
| Example 98 | 565 | 4.73 | 6.80 | 197 |
| Example 99 | 579 | 4.79 | 6.73 | 220 |
| Example 100 | 595 | 4.70 | 6.70 | 210 |
| Example 101 | 620 | 4.76 | 6.69 | 240 |
| Example 102 | 636 | 4.73 | 6.71 | 256 |
| Example 103 | 655 | 4.80 | 6.73 | 224 |
| Example 104 | 660 | 4.73 | 6.69 | 213 |
| Example 105 | 673 | 4.81 | 6.75 | 242 |
| Example 106 | 700 | 4.79 | 6.73 | 222 |
| Example 107 | 689 | 4.76 | 6.72 | 213 |
| Example 108 | 690 | 4.73 | 6.80 | 197 |
| Comparative | NPB | 5.32 | 6.18 | 172 |
| Example 8 | | | | |
| Comparative | HT1 | 5.36 | 6.14 | 171 |
| Example 9 | | | | |
| Comparative | HT2 | 5.30 | 6.18 | 182 |
| Example 10 | | | | |
| Comparative | HT3 | 5.33 | 6.16 | 167 |
| Example 11 | | | | |
| Comparative | HT4 | 5.30 | 6.10 | 178 |
| Example 12 | | | | |
| Comparative | HT5 | 5.20 | 6.22 | 168 |
| Example 13 | | | | |
| Comparative | HT6 | 5.19 | 6.22 | 149 |
| Example 14 | | | | |

As seen from the results of Table 6, the organic light emitting devices of Examples 55 to 108 using the heterocyclic compound of the present disclosure when forming the light emitting auxiliary layer were capable of delocalizing the HOMO energy level and thereby stabilizing HOMO energy by having the substituents of Chemical Formula 2 and Chemical Formula 3. Accordingly, electrons crossing over from the opposite side of the electron transport layer was effectively prevented, and it was seen that lowered driving voltage, and more superior light emission efficiency and lifetime were obtained compared to in the organic light emitting devices of Comparative Examples 8 to 14 when forming the light emitting auxiliary layer.

### REFERENCE NUMERAL

100: Substrate
200: Positive electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative electrode

1. A heterocyclic compound represented by the following Chemical Formula 1: 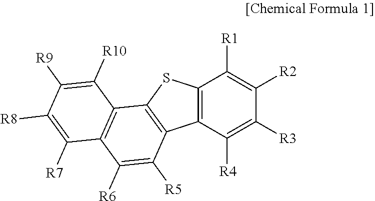
wherein, in Chemical Formula 1,
R1 to R10 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; a group represented by the following Chemical Formula 2; and a group represented by the following Chemical Formula 3, or two or more groups adjacent to each other bond to each other to form a substituted or unsubstituted C6 to C60 aromatic hydrocarbon ring; or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
at least one of R1 to R4 is a group represented by the following Chemical Formula 2; or a group represented by the following Chemical Formula 3; and
at least one of R5 to R10 is a group represented by the following Chemical Formula 2; or a group represented by the following Chemical Formula 3, 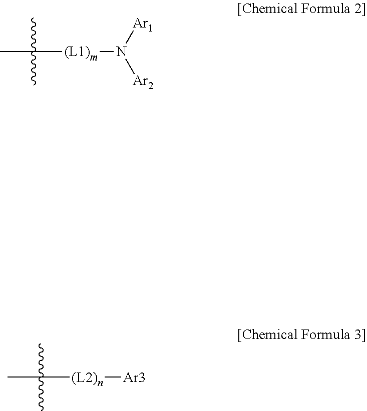
in Chemical Formula 2 and Chemical Formula 3,
Ar1 to Ar3 are the same as or different from each other, and each independently a substituted or unsubstituted C6 to C60 aryl group; or a substituted or unsubstituted C2 to C60 heteroaryl group;
L1 and L2 are the same as or different from each other, and each independently a direct bond; a substituted or unsubstituted C6 to C60 arylene group; or a substituted or unsubstituted C2 to C60 heteroarylene group; and
m and n are each an integer of 0 to 5, and when m is 2 or greater, L1s are the same as or different from each other, and when n is 2 or greater, L2s are the same as or different from each other.

2. The heterocyclic compound of claim 1, wherein Chemical Formula 1 is represented by the following Chemical Formula 4 or Chemical Formula 5: 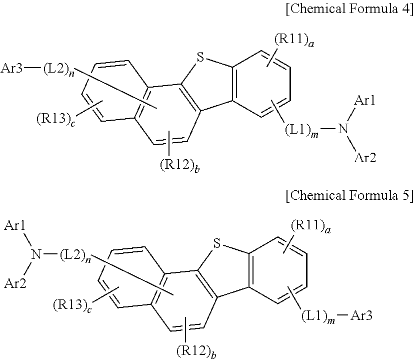
in Chemical Formulas 4 and 5,
R11 to R13 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, or two or more groups adjacent to each other bond to each other to form a substituted or unsubstituted C6 to C60 aromatic hydrocarbon ring; or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
a is an integer of 0 to 3, and when a is 2 or greater, Riis are the same as or different from each other;
b is an integer of 0 to 2, and when b is 2 or greater, R12s are the same as or different from each other,
c is an integer of 0 to 4, and when c is 2 or greater, R13s are the same as or different from each other;
Ar1, Ar2, L1 and m have the same definitions as in Chemical Formula 2; and
Ar3, L2 and n have the same definitions as in Chemical Formula 3.

3. The heterocyclic compound of claim 1, wherein Chemical Formula 1 is represented by the following Chemical Formula 6 or Chemical Formula 7: 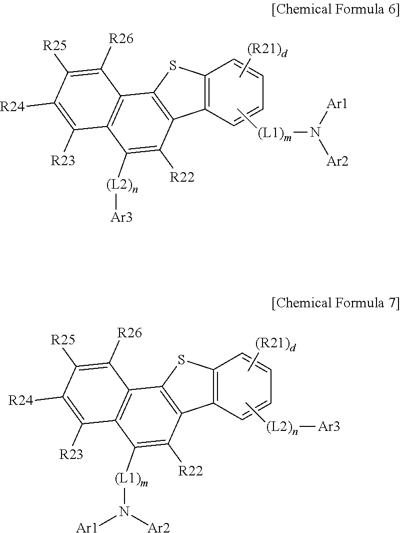
in Chemical Formulas 6 and 7,
R21 to R26 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, or two or more groups adjacent to each other bond to each other to form a substituted or unsubstituted C6 to C60 aromatic hydrocarbon ring; or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
d is an integer of 0 to 3, and when d is 2 or greater, R21s are the same as or different from each other;
Ar1, Ar2, L1 and m have the same definitions as in Chemical Formula 2; and
Ar3, L2 and n have the same definitions as in Chemical Formula 3.

4. The heterocyclic compound of claim 1, wherein Ar1 and Ar2 are the same as or different from each other, and each independently a substituted or unsubstituted C6 to C30 aryl group; or a substituted or unsubstituted C2 to C30 heteroaryl group.

5. The heterocyclic compound of claim 1, wherein Ar3 is a substituted or unsubstituted C6 to C30 aryl group; or a substituted or unsubstituted C2 to C30 heteroaryl group.

6. The heterocyclic compound of claim 1, wherein the heterocyclic compound represented by Chemical Formula 1 does not include deuterium as a substituent, or a content of deuterium is from 1% to 100% with respect to a total number of hydrogen atoms and deuterium atoms.

7. The heterocyclic compound of claim 1, wherein Chemical Formula 1 is represented by any one of the following compounds:

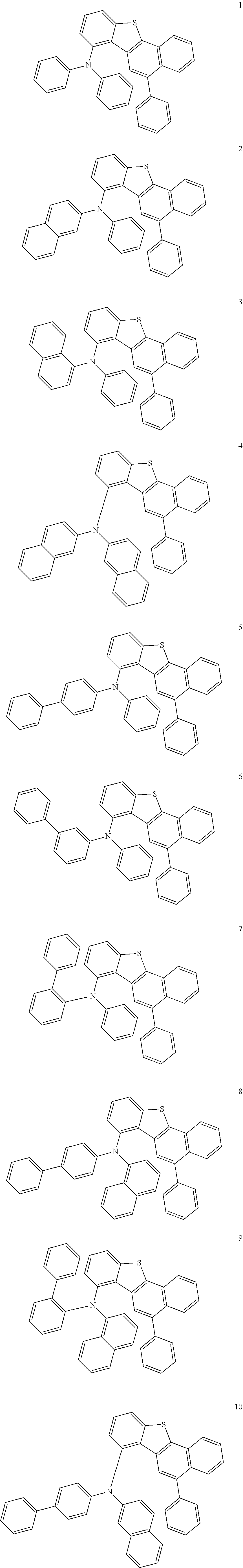

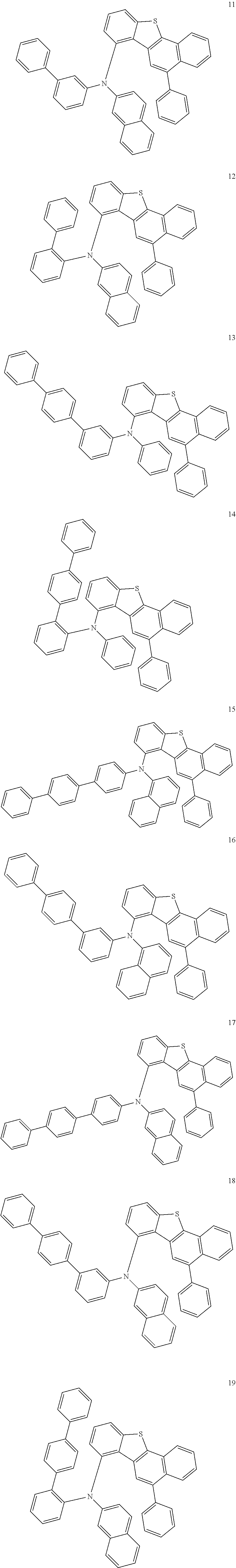

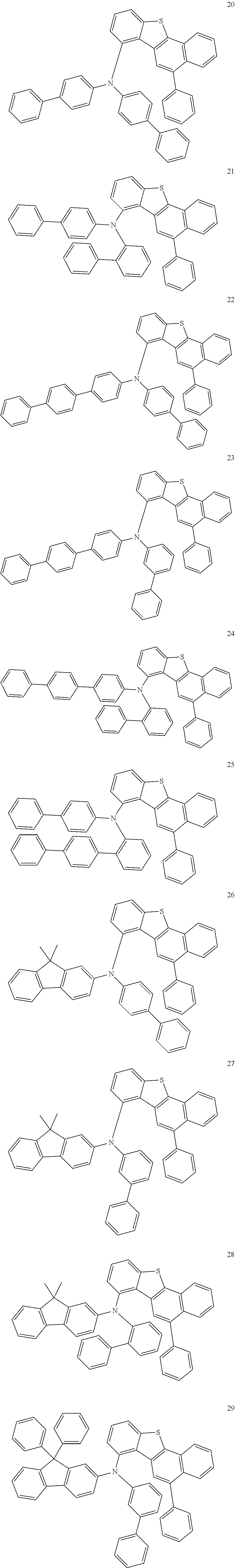

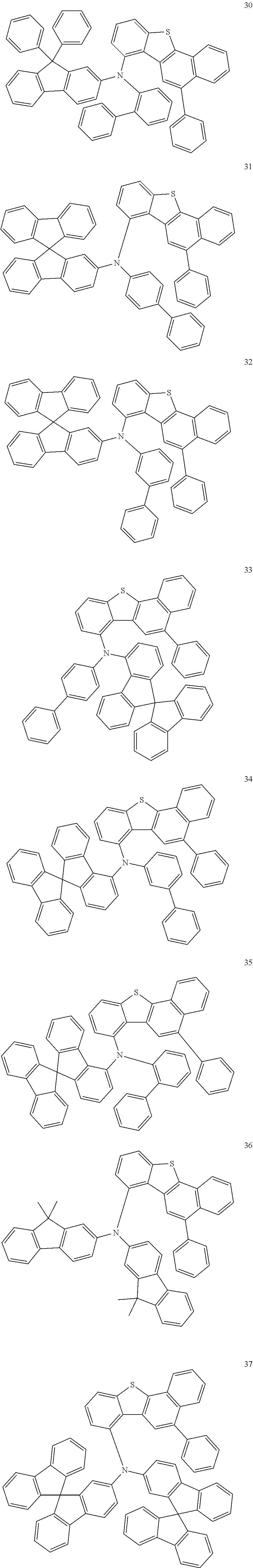

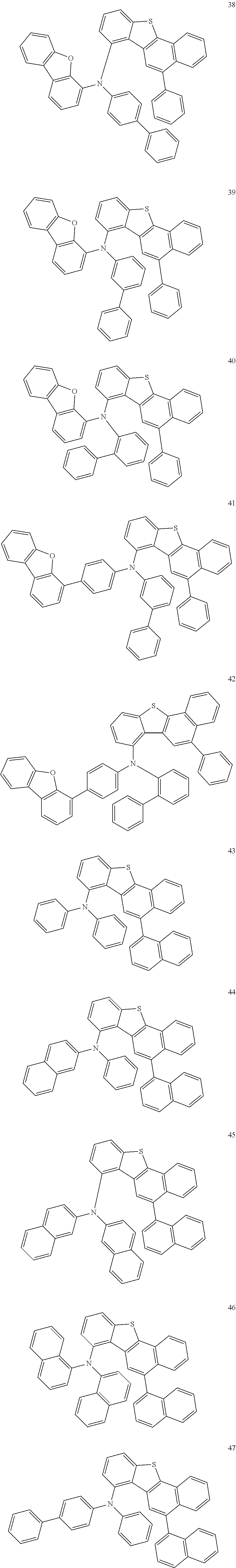

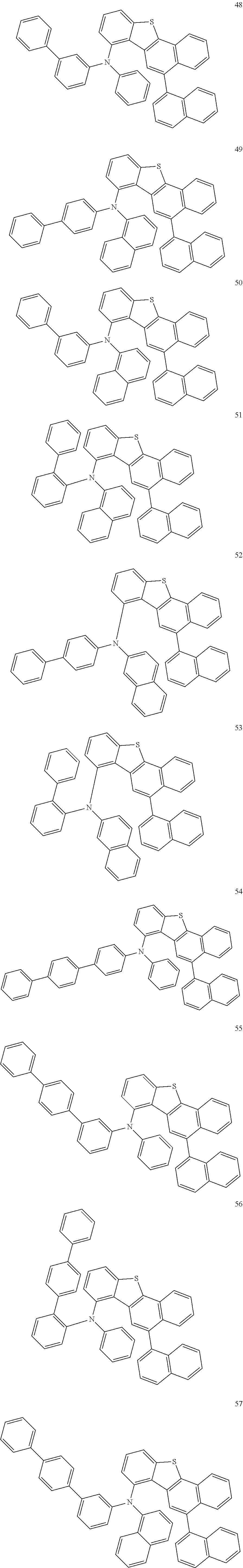

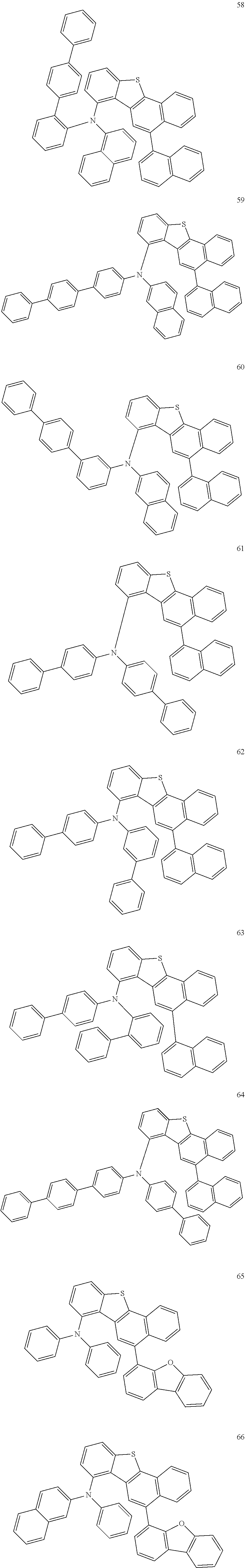

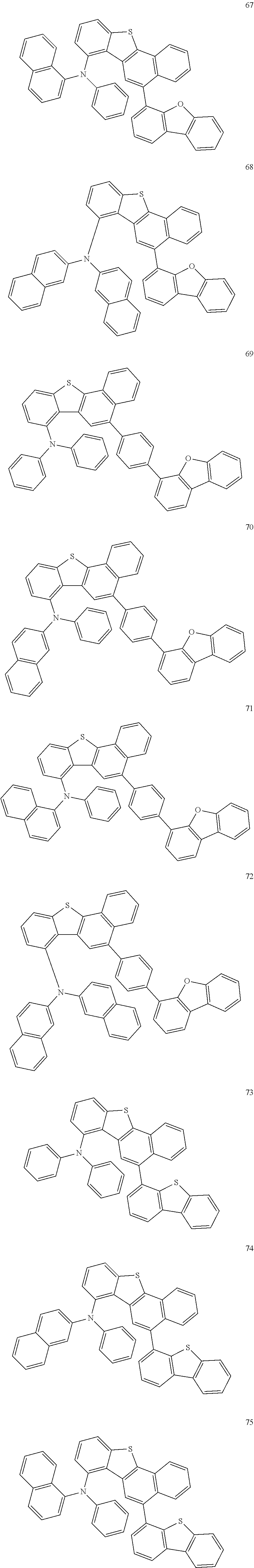

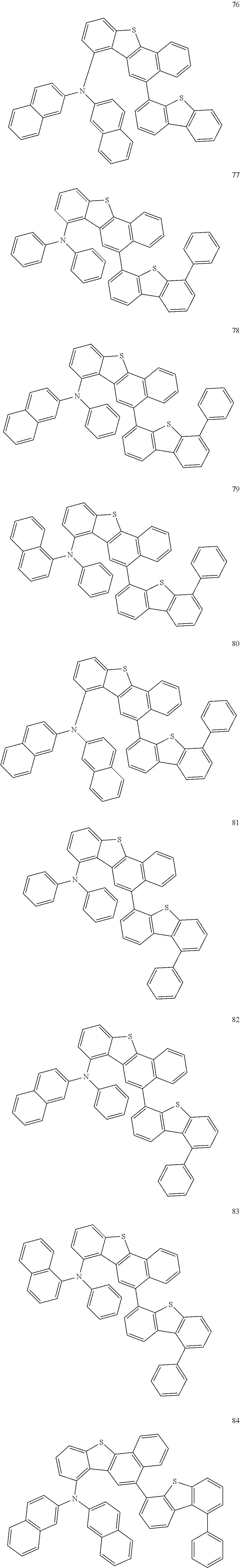

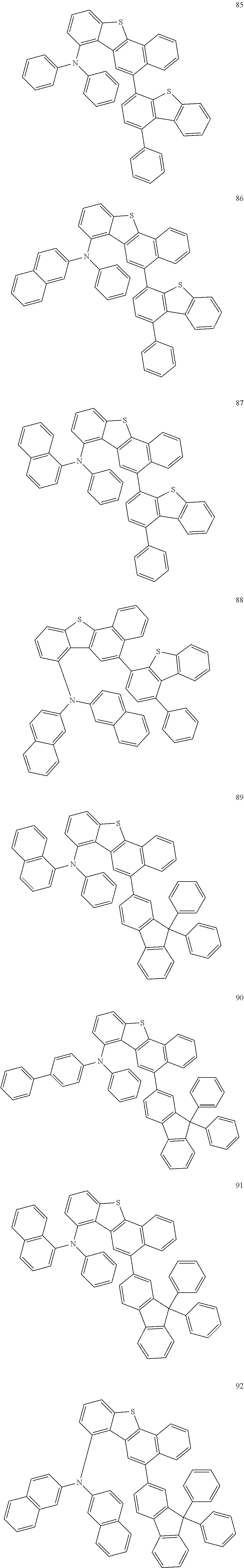

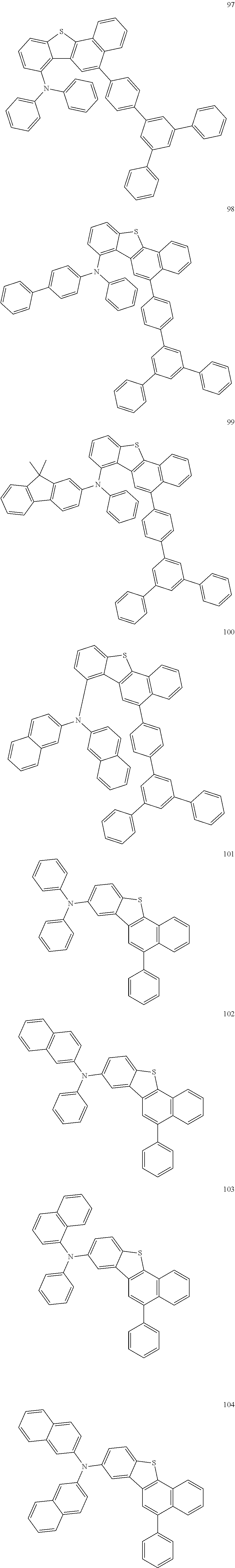

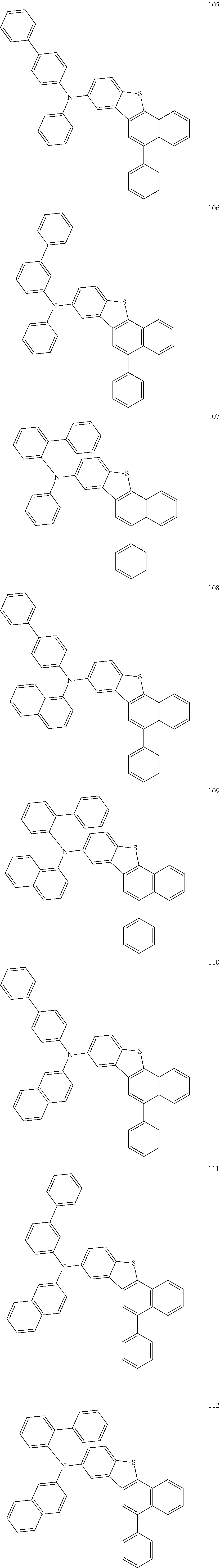

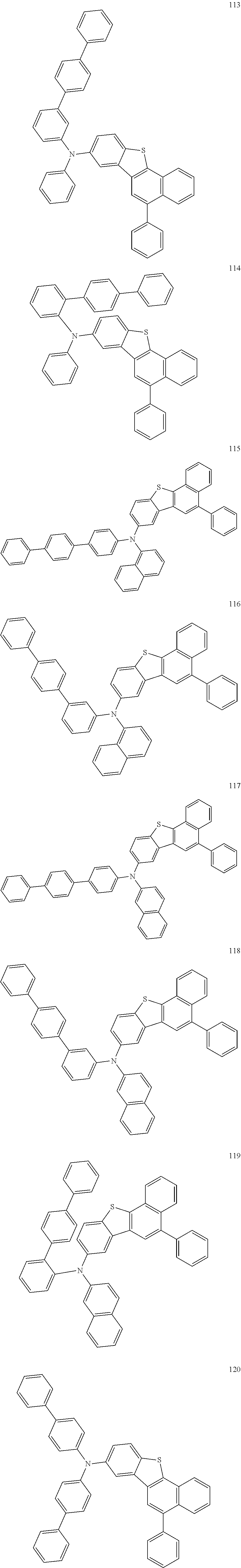

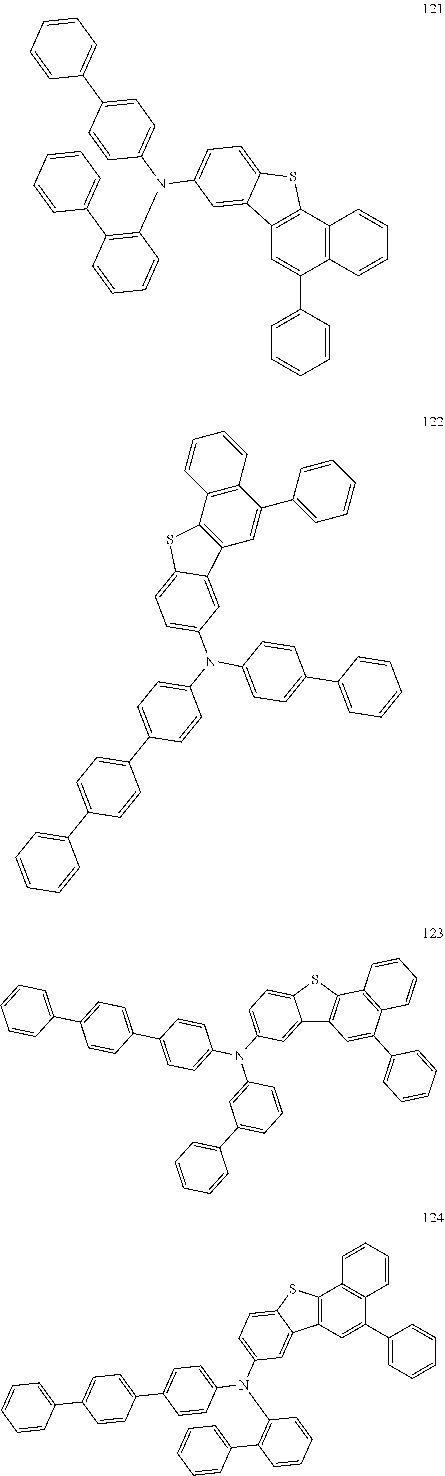 [SMILES: c1ccc(-c2ccc(N(c3ccc4sc5c6ccccc6c(-c6ccccc6)cc5c4c3)c3ccccc3-c3ccccc3)cc2)cc1 ; c1ccc(-c2ccc(-c3ccc(N(c4ccc(-c5ccccc5)cc4)c4ccc5sc6c7ccccc7c(-c7ccccc7)cc6c5c4)cc3)cc2)cc1 ; c1ccc(-c2ccc(-c3ccc(N(c4cccc(-c5ccccc5)c4)c4ccc5sc6c7ccccc7c(-c7ccccc7)cc6c5c4)cc3)cc2)cc1 ; c1ccc(-c2ccc(-c3ccc(N(c4ccc5sc6c7ccccc7c(-c7ccccc7)cc6c5c4)c4ccccc4-c4ccccc4)cc3)cc2)cc1]

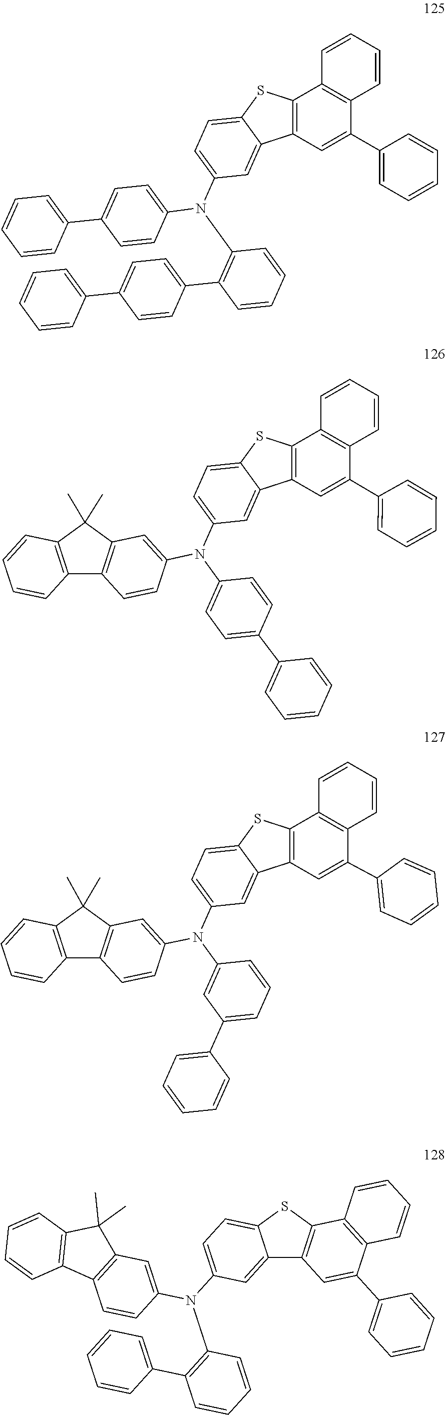 [SMILES: c1ccc(-c2ccc(-c3ccccc3N(c3ccc(-c4ccccc4)cc3)c3ccc4sc5c6ccccc6c(-c6ccccc6)cc5c4c3)cc2)cc1 ; CC1(C)c2ccccc2-c2ccc(N(c3ccc(-c4ccccc4)cc3)c3ccc4sc5c6ccccc6c(-c6ccccc6)cc5c4c3)cc21 ; CC1(C)c2ccccc2-c2ccc(N(c3cccc(-c4ccccc4)c3)c3ccc4sc5c6ccccc6c(-c6ccccc6)cc5c4c3)cc21 ; CC1(C)c2ccccc2-c2ccc(N(c3ccc4sc5c6ccccc6c(-c6ccccc6)cc5c4c3)c3ccccc3-c3ccccc3)cc21]

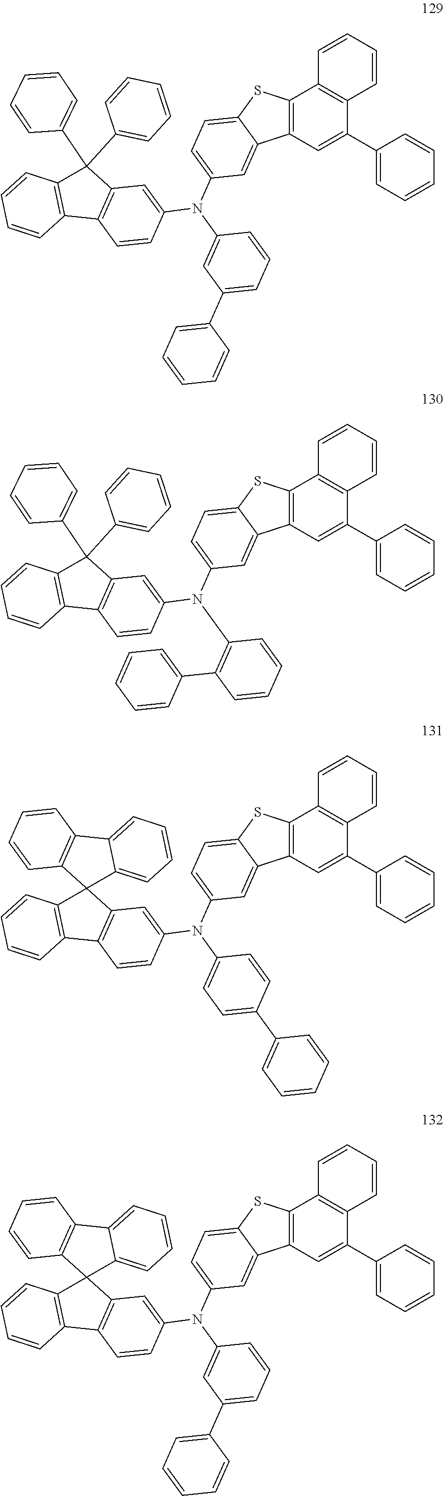 [SMILES: c1ccc(-c2cccc(N(c3ccc4c(c3)C(c3ccccc3)(c3ccccc3)c3ccccc3-4)c3ccc4sc5c6ccccc6c(-c6ccccc6)cc5c4c3)c2)cc1 ; c1ccc(-c2ccccc2N(c2ccc3c(c2)C(c2ccccc2)(c2ccccc2)c2ccccc2-3)c2ccc3sc4c5ccccc5c(-c5ccccc5)cc4c3c2)cc1 ; c1ccc(-c2ccc(N(c3ccc4c(c3)C3(c5ccccc5-c5ccccc53)c3ccccc3-4)c3ccc4sc5c6ccccc6c(-c6ccccc6)cc5c4c3)cc2)cc1 ; c1ccc(-c2cccc(N(c3ccc4c(c3)C3(c5ccccc5-c5ccccc53)c3ccccc3-4)c3ccc4sc5c6ccccc6c(-c6ccccc6)cc5c4c3)c2)cc1]

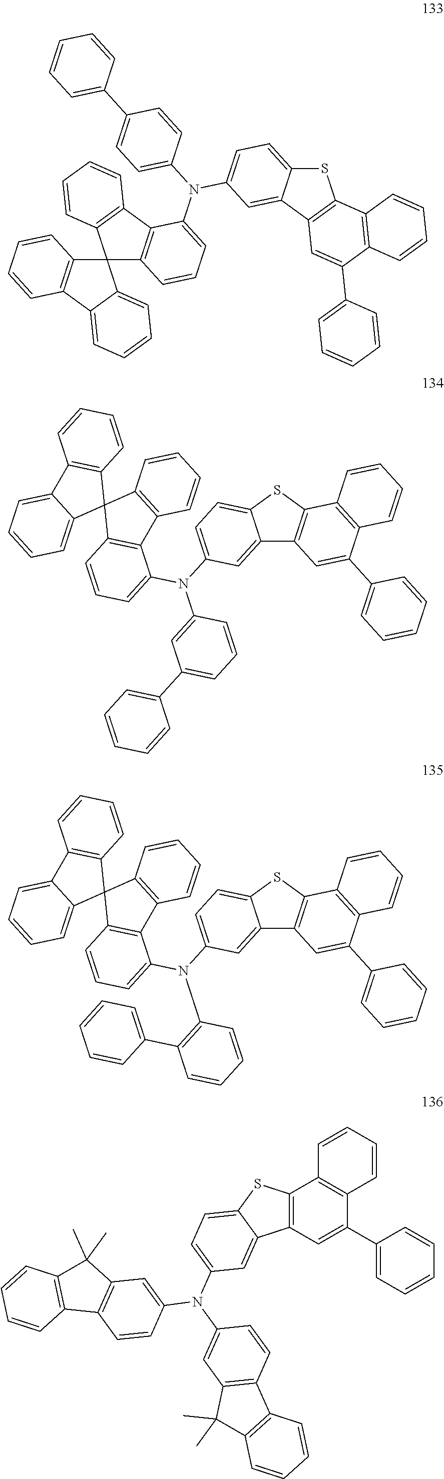 [SMILES: c1ccc(-c2ccc(N(c3ccc4sc5c6ccccc6c(-c6ccccc6)cc5c4c3)c3cccc4c3-c3ccccc3C43c4ccccc4-c4ccccc43)cc2)cc1 ; c1ccc(-c2cccc(N(c3ccc4sc5c6ccccc6c(-c6ccccc6)cc5c4c3)c3cccc4c3-c3ccccc3C43c4ccccc4-c4ccccc43)c2)cc1 ; c1ccc(-c2ccccc2N(c2ccc3sc4c5ccccc5c(-c5ccccc5)cc4c3c2)c2cccc3c2-c2ccccc2C32c3ccccc3-c3ccccc32)cc1 ; CC1(C)c2ccccc2-c2ccc(N(c3ccc4c(c3)C(C)(C)c3ccccc3-4)c3ccc4sc5c6ccccc6c(-c6ccccc6)cc5c4c3)cc21]

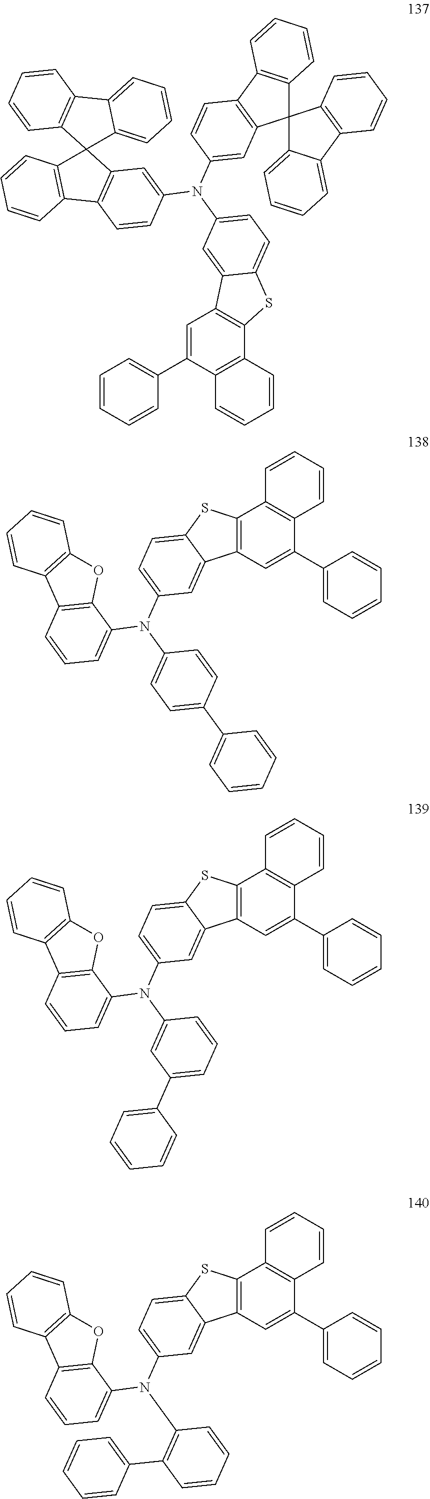

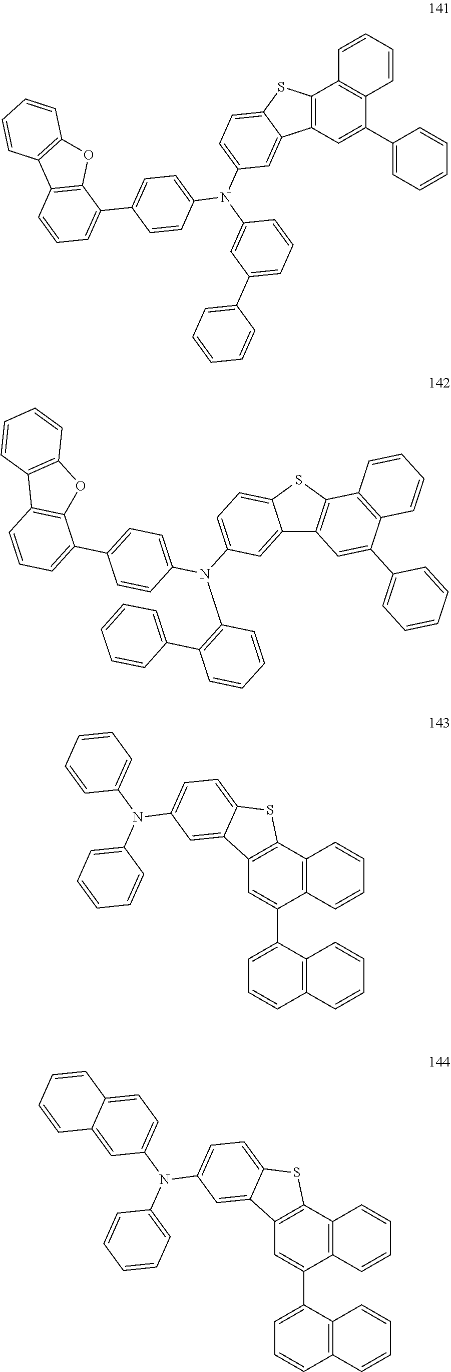 [SMILES: c1ccc(-c2cccc(N(c3ccc(-c4cccc5c4oc4ccccc45)cc3)c3ccc4sc5c6ccccc6c(-c6ccccc6)cc5c4c3)c2)cc1 ; c1ccc(-c2ccccc2N(c2ccc(-c3cccc4c3oc3ccccc34)cc2)c2ccc3sc4c5ccccc5c(-c5ccccc5)cc4c3c2)cc1 ; c1ccc(N(c2ccccc2)c2ccc3sc4c5ccccc5c(-c5cccc6ccccc56)cc4c3c2)cc1 ; c1ccc(N(c2ccc3ccccc3c2)c2ccc3sc4c5ccccc5c(-c5cccc6ccccc56)cc4c3c2)cc1]

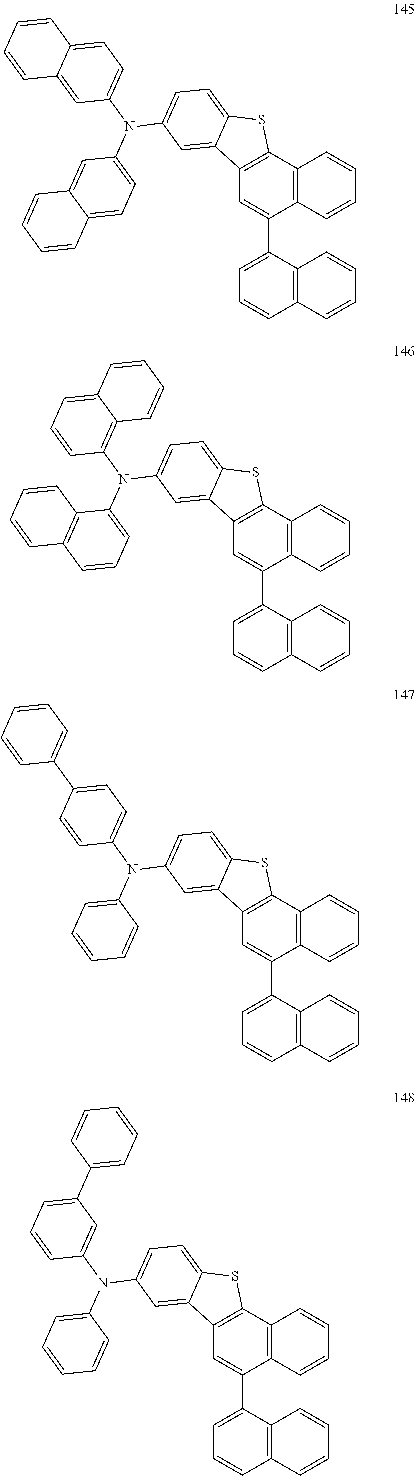 [SMILES: c1ccc2cc(N(c3ccc4ccccc4c3)c3ccc4sc5c6ccccc6c(-c6cccc7ccccc67)cc5c4c3)ccc2c1 ; c1ccc2c(-c3cc4c5cc(N(c6cccc7ccccc67)c6cccc7ccccc67)ccc5sc4c4ccccc34)cccc2c1 ; c1ccc(-c2ccc(N(c3ccccc3)c3ccc4sc5c6ccccc6c(-c6cccc7ccccc67)cc5c4c3)cc2)cc1 ; c1ccc(-c2cccc(N(c3ccccc3)c3ccc4sc5c6ccccc6c(-c6cccc7ccccc67)cc5c4c3)c2)cc1]

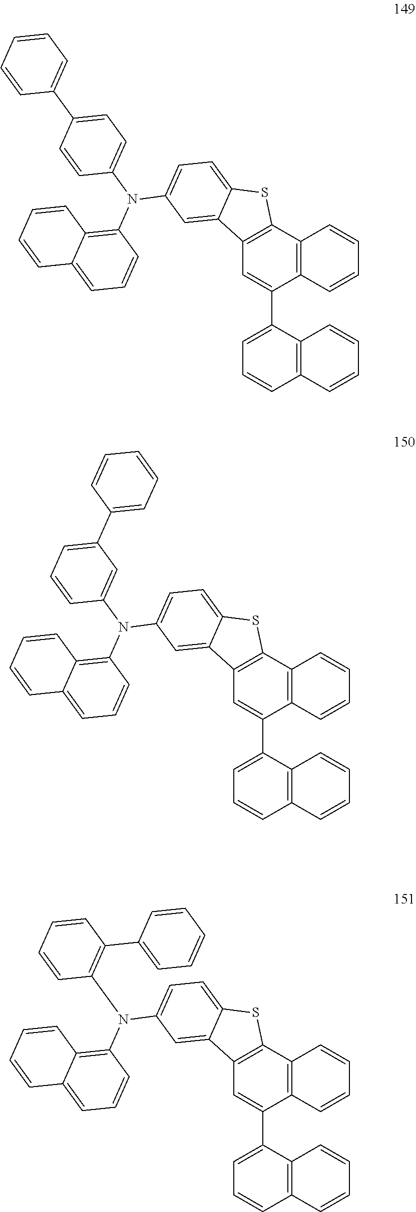 [SMILES: c1ccc(-c2ccc(N(c3ccc4sc5c6ccccc6c(-c6cccc7ccccc67)cc5c4c3)c3cccc4ccccc34)cc2)cc1 ; c1ccc(-c2cccc(N(c3ccc4sc5c6ccccc6c(-c6cccc7ccccc67)cc5c4c3)c3cccc4ccccc34)c2)cc1 ; c1ccc(-c2ccccc2N(c2ccc3sc4c5ccccc5c(-c5cccc6ccccc56)cc4c3c2)c2cccc3ccccc23)cc1]

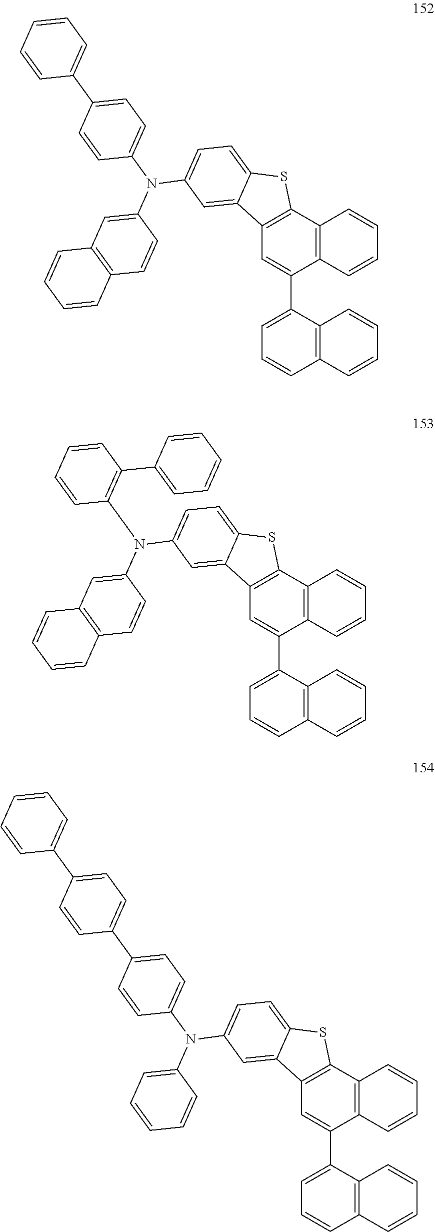 [SMILES: c1ccc(-c2ccc(N(c3ccc4ccccc4c3)c3ccc4sc5c6ccccc6c(-c6cccc7ccccc67)cc5c4c3)cc2)cc1 ; c1ccc(-c2ccccc2N(c2ccc3ccccc3c2)c2ccc3sc4c5ccccc5c(-c5cccc6ccccc56)cc4c3c2)cc1 ; c1ccc(-c2ccc(-c3ccc(N(c4ccccc4)c4ccc5sc6c7ccccc7c(-c7cccc8ccccc78)cc6c5c4)cc3)cc2)cc1]

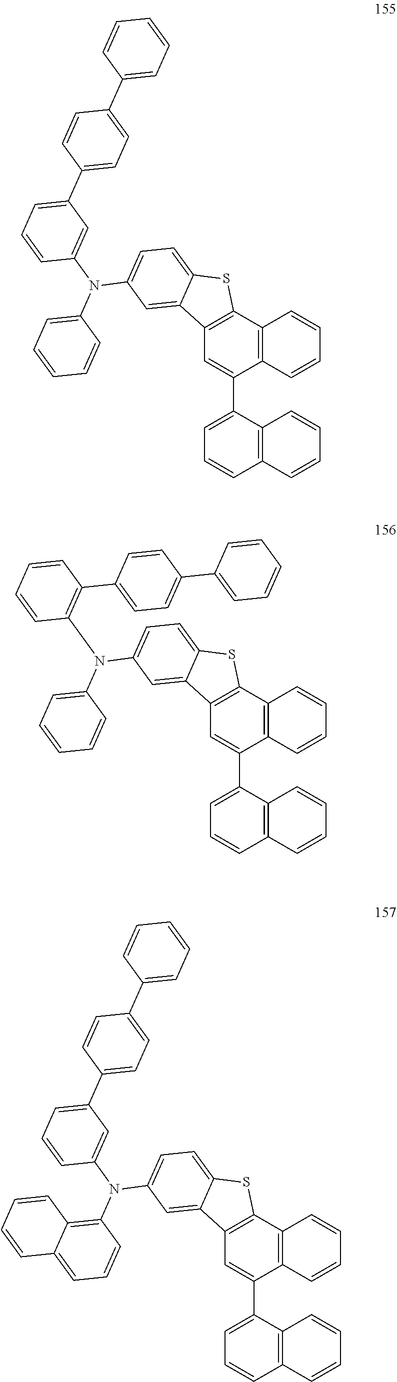 [SMILES: c1ccc(-c2ccc(-c3cccc(N(c4ccccc4)c4ccc5sc6c7ccccc7c(-c7cccc8ccccc78)cc6c5c4)c3)cc2)cc1 ; c1ccc(-c2ccc(-c3ccccc3N(c3ccccc3)c3ccc4sc5c6ccccc6c(-c6cccc7ccccc67)cc5c4c3)cc2)cc1 ; c1ccc(-c2ccc(-c3cccc(N(c4ccc5sc6c7ccccc7c(-c7cccc8ccccc78)cc6c5c4)c4cccc5ccccc45)c3)cc2)cc1]

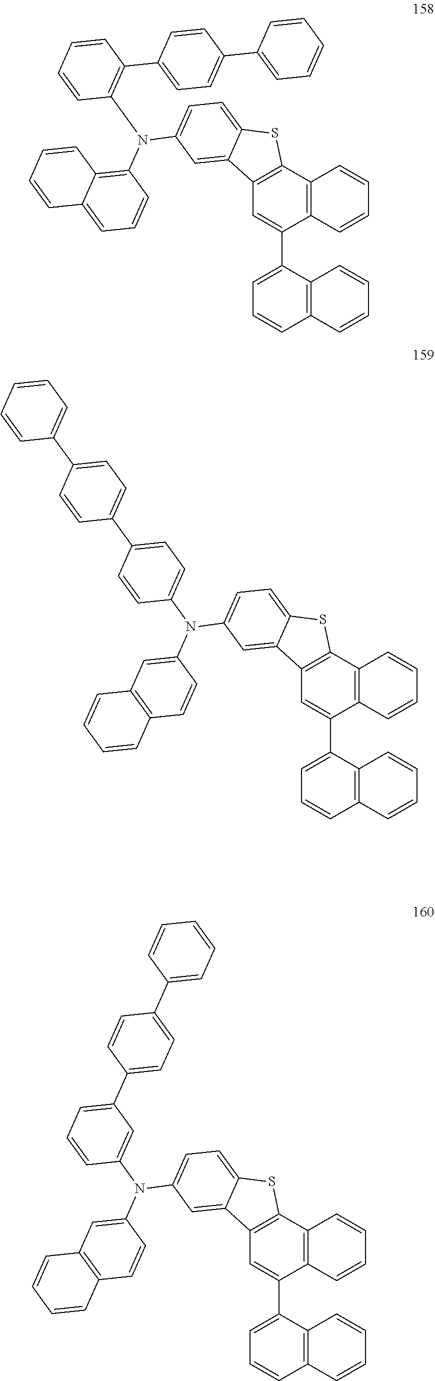 [SMILES: c1ccc(-c2ccc(-c3ccccc3N(c3ccc4sc5c6ccccc6c(-c6cccc7ccccc67)cc5c4c3)c3cccc4ccccc34)cc2)cc1 ; c1ccc(-c2ccc(-c3ccc(N(c4ccc5ccccc5c4)c4ccc5sc6c7ccccc7c(-c7cccc8ccccc78)cc6c5c4)cc3)cc2)cc1 ; c1ccc(-c2ccc(-c3cccc(N(c4ccc5ccccc5c4)c4ccc5sc6c7ccccc7c(-c7cccc8ccccc78)cc6c5c4)c3)cc2)cc1]

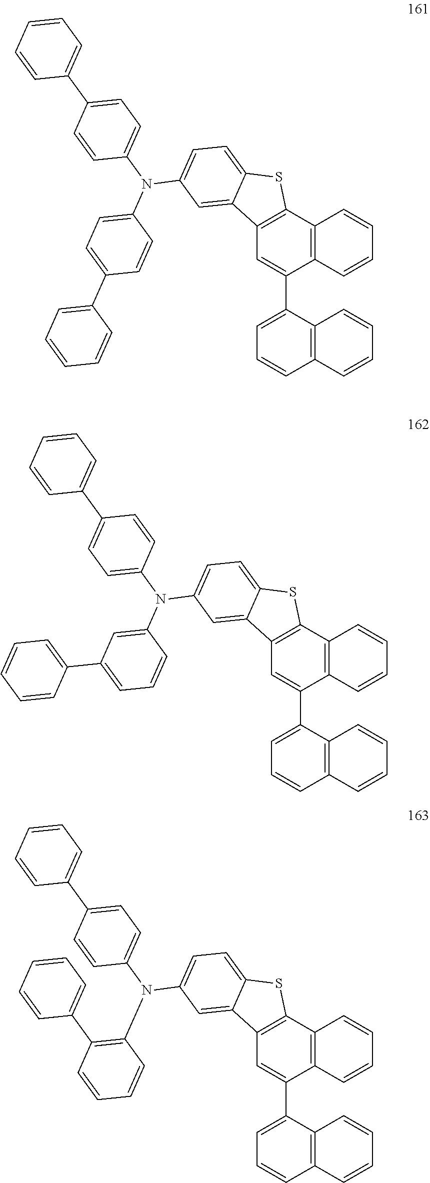 [SMILES: c1ccc(-c2ccc(N(c3ccc(-c4ccccc4)cc3)c3ccc4sc5c6ccccc6c(-c6cccc7ccccc67)cc5c4c3)cc2)cc1 ; c1ccc(-c2ccc(N(c3cccc(-c4ccccc4)c3)c3ccc4sc5c6ccccc6c(-c6cccc7ccccc67)cc5c4c3)cc2)cc1 ; c1ccc(-c2ccc(N(c3ccc4sc5c6ccccc6c(-c6cccc7ccccc67)cc5c4c3)c3ccccc3-c3ccccc3)cc2)cc1]

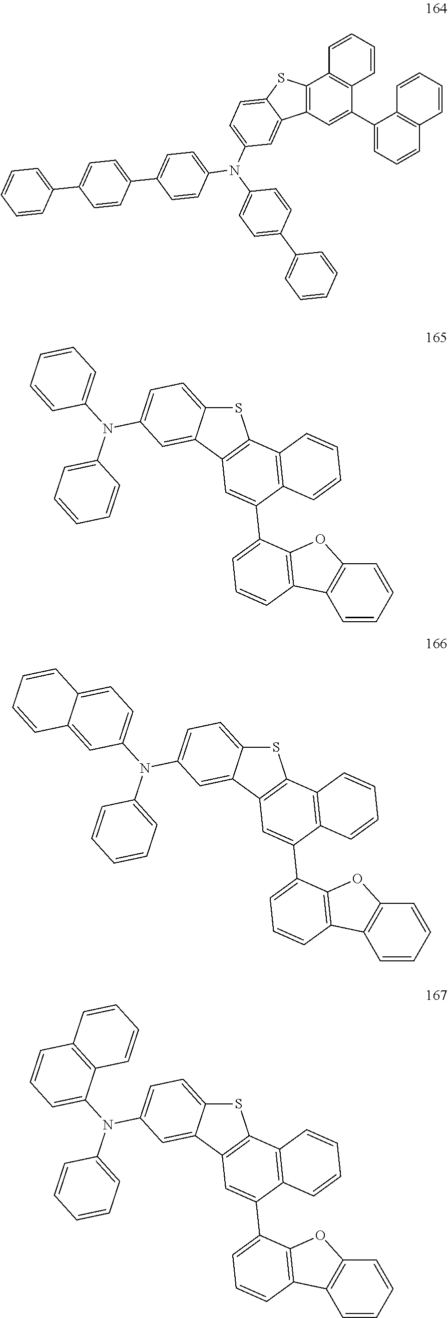 [SMILES: c1ccc(-c2ccc(-c3ccc(N(c4ccc(-c5ccccc5)cc4)c4ccc5sc6c7ccccc7c(-c7cccc8ccccc78)cc6c5c4)cc3)cc2)cc1 ; c1ccc(N(c2ccccc2)c2ccc3sc4c5ccccc5c(-c5cccc6c5oc5ccccc56)cc4c3c2)cc1 ; c1ccc(N(c2ccc3ccccc3c2)c2ccc3sc4c5ccccc5c(-c5cccc6c5oc5ccccc56)cc4c3c2)cc1 ; c1ccc(N(c2ccc3sc4c5ccccc5c(-c5cccc6c5oc5ccccc56)cc4c3c2)c2cccc3ccccc23)cc1]

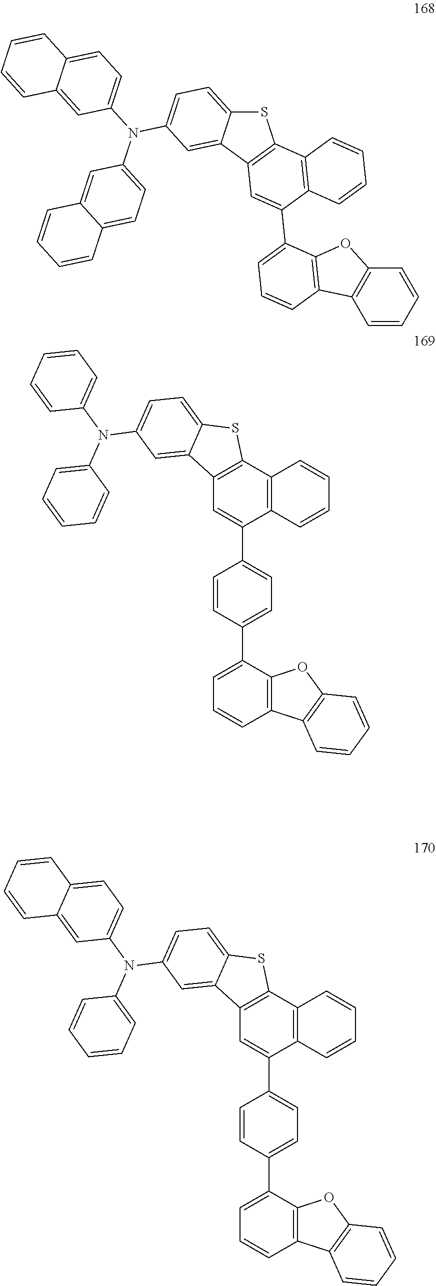 [SMILES: c1ccc2cc(N(c3ccc4ccccc4c3)c3ccc4sc5c6ccccc6c(-c6cccc7c6oc6ccccc67)cc5c4c3)ccc2c1 ; c1ccc(N(c2ccccc2)c2ccc3sc4c5ccccc5c(-c5ccc(-c6cccc7c6oc6ccccc67)cc5)cc4c3c2)cc1 ; c1ccc(N(c2ccc3ccccc3c2)c2ccc3sc4c5ccccc5c(-c5ccc(-c6cccc7c6oc6ccccc67)cc5)cc4c3c2)cc1]

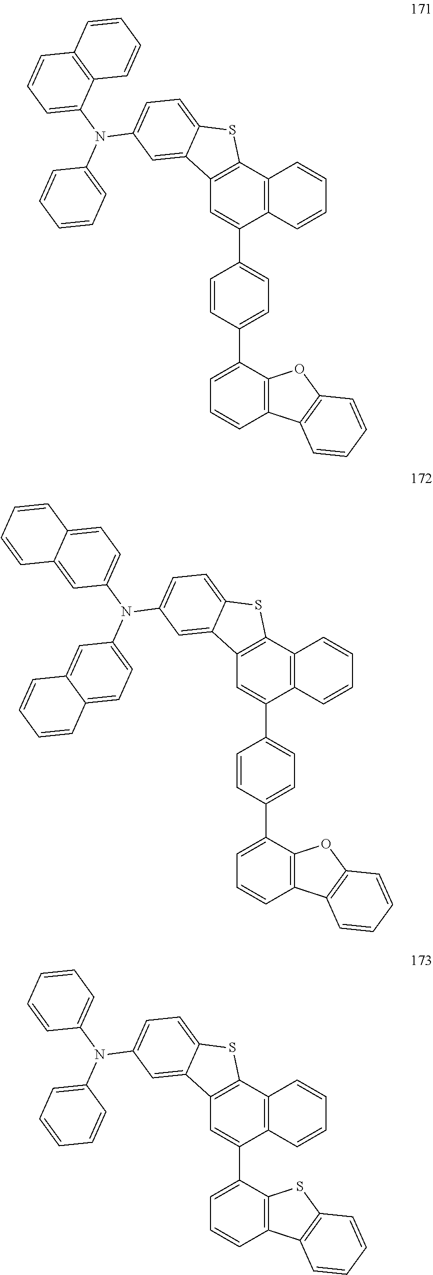 [SMILES: c1ccc(N(c2ccc3sc4c5ccccc5c(-c5ccc(-c6cccc7c6oc6ccccc67)cc5)cc4c3c2)c2cccc3ccccc23)cc1 ; c1ccc2cc(N(c3ccc4ccccc4c3)c3ccc4sc5c6ccccc6c(-c6ccc(-c7cccc8c7oc7ccccc78)cc6)cc5c4c3)ccc2c1 ; c1ccc(N(c2ccccc2)c2ccc3sc4c5ccccc5c(-c5cccc6c5sc5ccccc56)cc4c3c2)cc1]

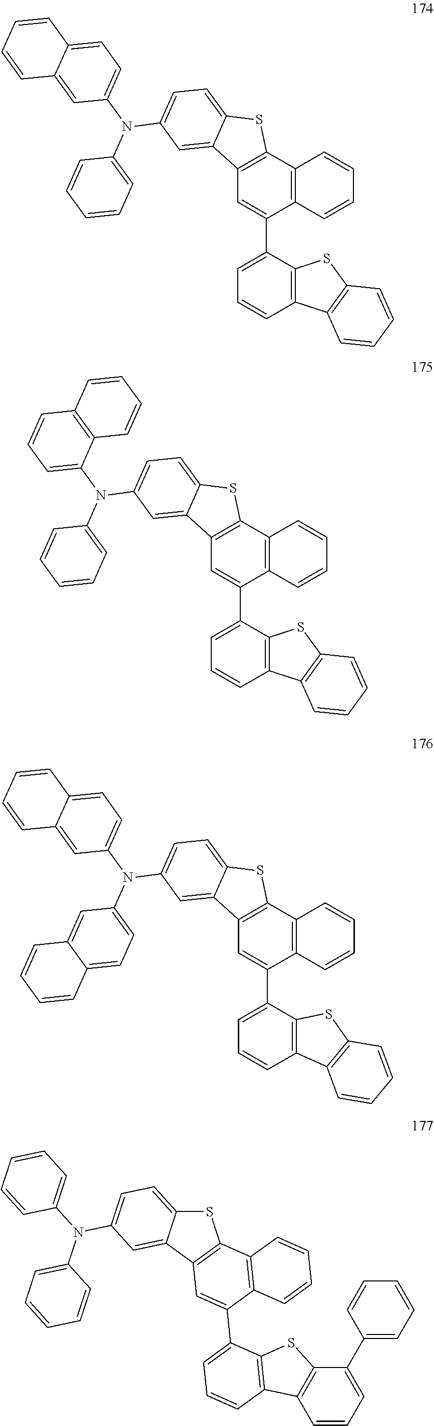 [SMILES: c1ccc(N(c2ccc3ccccc3c2)c2ccc3sc4c5ccccc5c(-c5cccc6c5sc5ccccc56)cc4c3c2)cc1 ; c1ccc(N(c2ccc3sc4c5ccccc5c(-c5cccc6c5sc5ccccc56)cc4c3c2)c2cccc3ccccc23)cc1 ; c1ccc2cc(N(c3ccc4ccccc4c3)c3ccc4sc5c6ccccc6c(-c6cccc7c6sc6ccccc67)cc5c4c3)ccc2c1 ; c1ccc(-c2cccc3c2sc2c(-c4cc5c6cc(N(c7ccccc7)c7ccccc7)ccc6sc5c5ccccc45)cccc23)cc1]

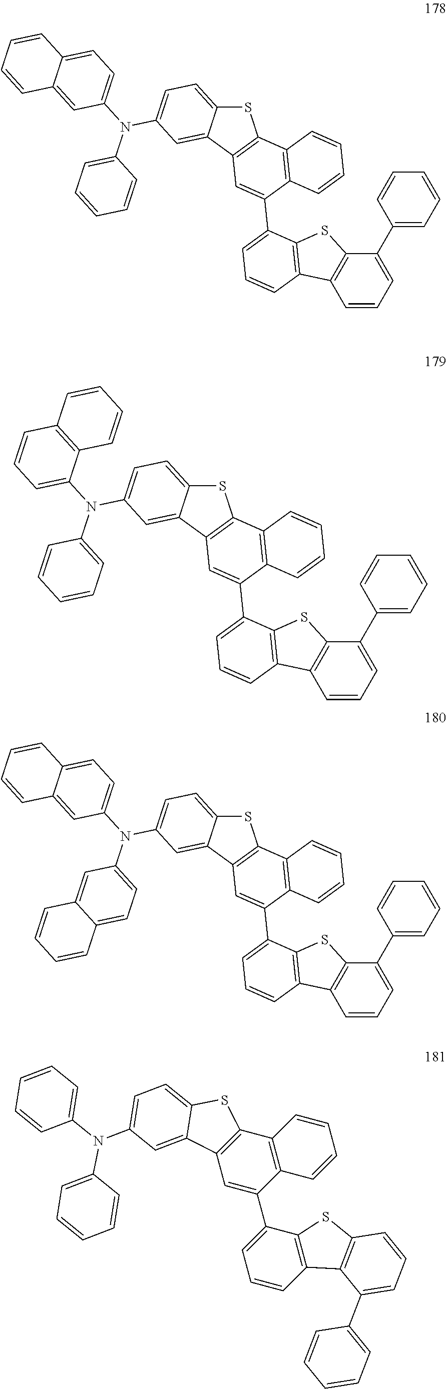 [SMILES: c1ccc(-c2cccc3c2sc2c(-c4cc5c6cc(N(c7ccccc7)c7ccc8ccccc8c7)ccc6sc5c5ccccc45)cccc23)cc1 ; c1ccc(-c2cccc3c2sc2c(-c4cc5c6cc(N(c7ccccc7)c7cccc8ccccc78)ccc6sc5c5ccccc45)cccc23)cc1 ; c1ccc(-c2cccc3c2sc2c(-c4cc5c6cc(N(c7ccc8ccccc8c7)c7ccc8ccccc8c7)ccc6sc5c5ccccc45)cccc23)cc1 ; c1ccc(-c2cccc3sc4c(-c5cc6c7cc(N(c8ccccc8)c8ccccc8)ccc7sc6c6ccccc56)cccc4c23)cc1]

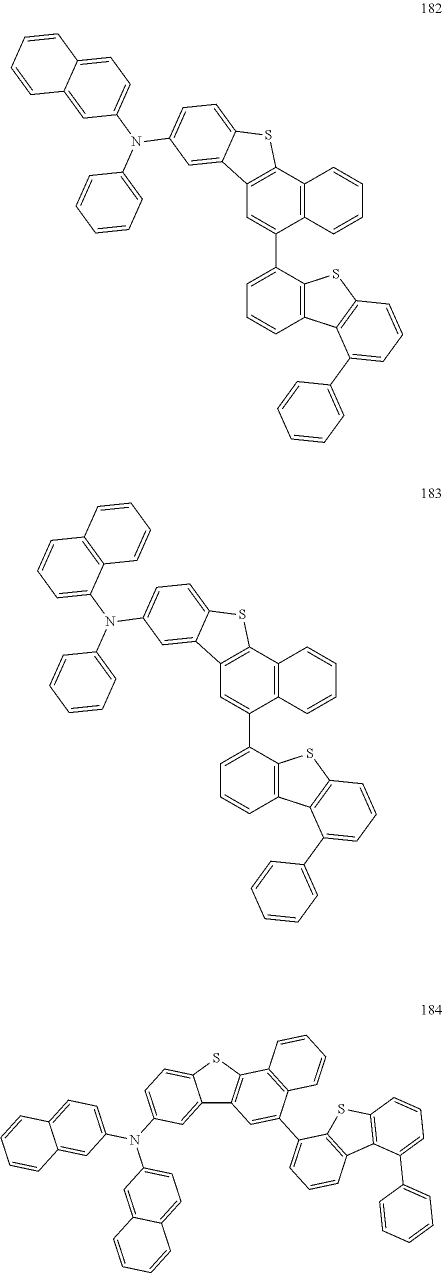 [SMILES: c1ccc(-c2cccc3sc4c(-c5cc6c7cc(N(c8ccccc8)c8ccc9ccccc9c8)ccc7sc6c6ccccc56)cccc4c23)cc1 ; c1ccc(-c2cccc3sc4c(-c5cc6c7cc(N(c8ccccc8)c8cccc9ccccc89)ccc7sc6c6ccccc56)cccc4c23)cc1 ; c1ccc(-c2cccc3sc4c(-c5cc6c7cc(N(c8ccc9ccccc9c8)c8ccc9ccccc9c8)ccc7sc6c6ccccc56)cccc4c23)cc1]

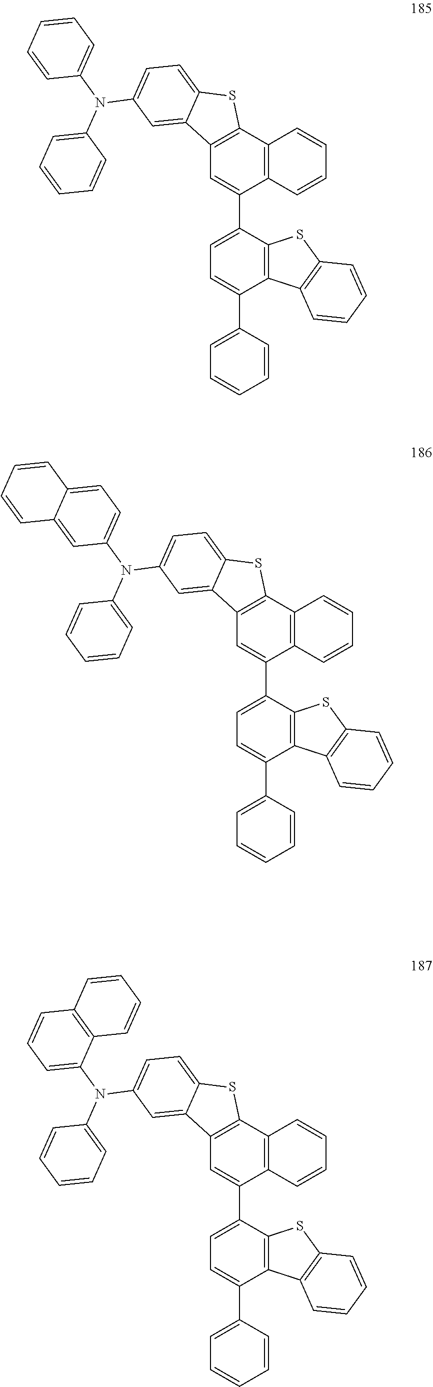 [SMILES: c1ccc(-c2ccc(-c3cc4c5cc(N(c6ccccc6)c6ccccc6)ccc5sc4c4ccccc34)c3sc4ccccc4c23)cc1 ; c1ccc(-c2ccc(-c3cc4c5cc(N(c6ccccc6)c6ccc7ccccc7c6)ccc5sc4c4ccccc34)c3sc4ccccc4c23)cc1 ; c1ccc(-c2ccc(-c3cc4c5cc(N(c6ccccc6)c6cccc7ccccc67)ccc5sc4c4ccccc34)c3sc4ccccc4c23)cc1]

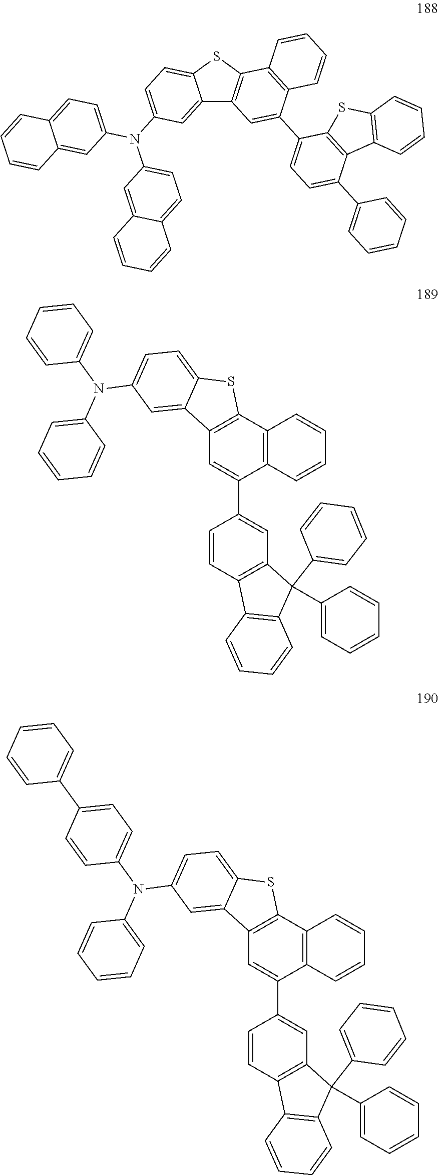 [SMILES: c1ccc(-c2ccc(-c3cc4c5cc(N(c6ccc7ccccc7c6)c6ccc7ccccc7c6)ccc5sc4c4ccccc34)c3sc4ccccc4c23)cc1 ; c1ccc(N(c2ccccc2)c2ccc3sc4c5ccccc5c(-c5ccc6c(c5)C(c5ccccc5)(c5ccccc5)c5ccccc5-6)cc4c3c2)cc1 ; c1ccc(-c2ccc(N(c3ccccc3)c3ccc4sc5c6ccccc6c(-c6ccc7c(c6)C(c6ccccc6)(c6ccccc6)c6ccccc6-7)cc5c4c3)cc2)cc1]

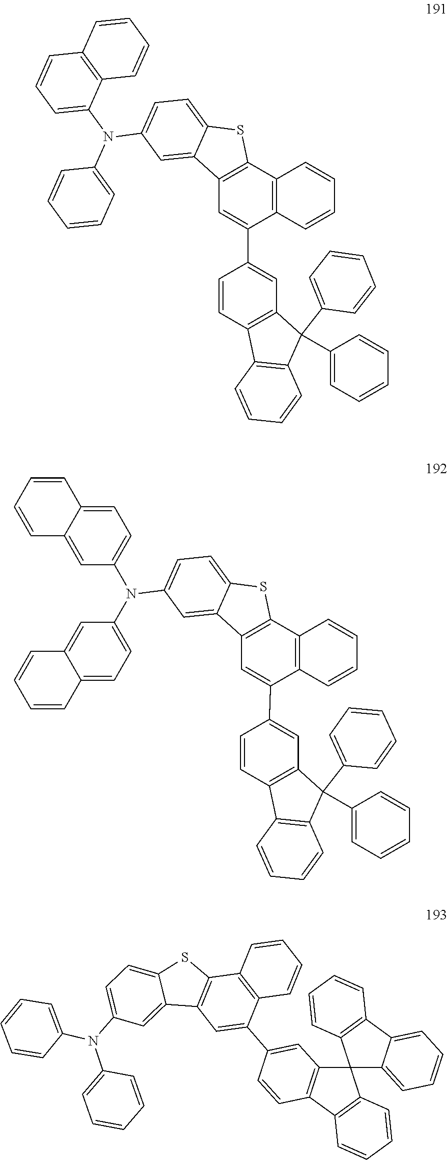 [SMILES: c1ccc(N(c2ccc3sc4c5ccccc5c(-c5ccc6c(c5)C(c5ccccc5)(c5ccccc5)c5ccccc5-6)cc4c3c2)c2cccc3ccccc23)cc1 ; c1ccc(C2(c3ccccc3)c3ccccc3-c3ccc(-c4cc5c6cc(N(c7ccc8ccccc8c7)c7ccc8ccccc8c7)ccc6sc5c5ccccc45)cc32)cc1 ; c1ccc(N(c2ccccc2)c2ccc3sc4c5ccccc5c(-c5ccc6c(c5)C5(c7ccccc7-c7ccccc75)c5ccccc5-6)cc4c3c2)cc1]

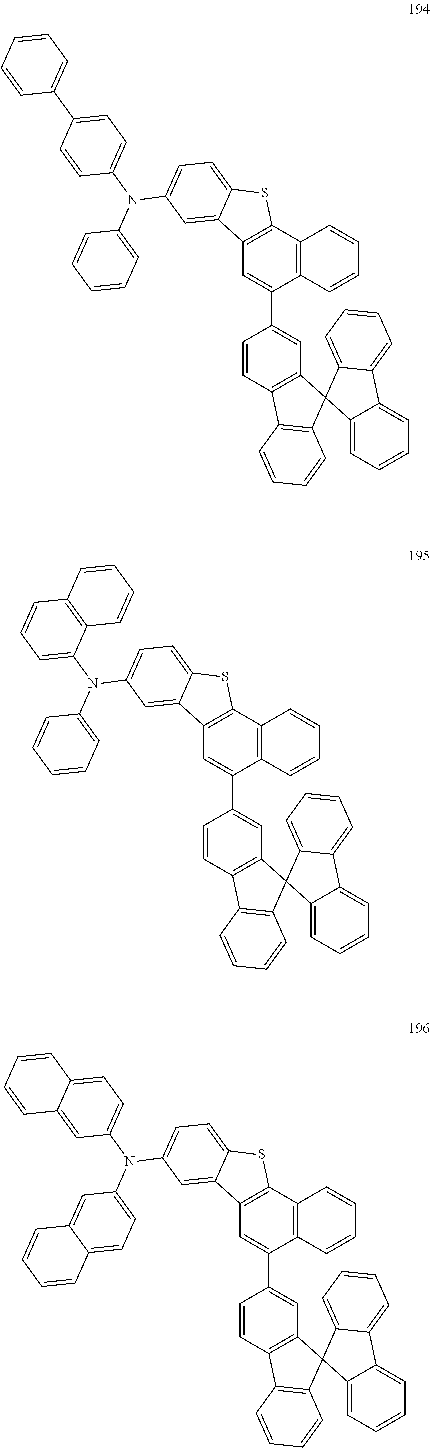 [SMILES: c1ccc(-c2ccc(N(c3ccccc3)c3ccc4sc5c6ccccc6c(-c6ccc7c(c6)C6(c8ccccc8-c8ccccc86)c6ccccc6-7)cc5c4c3)cc2)cc1 ; c1ccc(N(c2ccc3sc4c5ccccc5c(-c5ccc6c(c5)C5(c7ccccc7-c7ccccc75)c5ccccc5-6)cc4c3c2)c2cccc3ccccc23)cc1 ; c1ccc2c(c1)-c1ccccc1C21c2ccccc2-c2ccc(-c3cc4c5cc(N(c6ccc7ccccc7c6)c6ccc7ccccc7c6)ccc5sc4c4ccccc34)cc21]

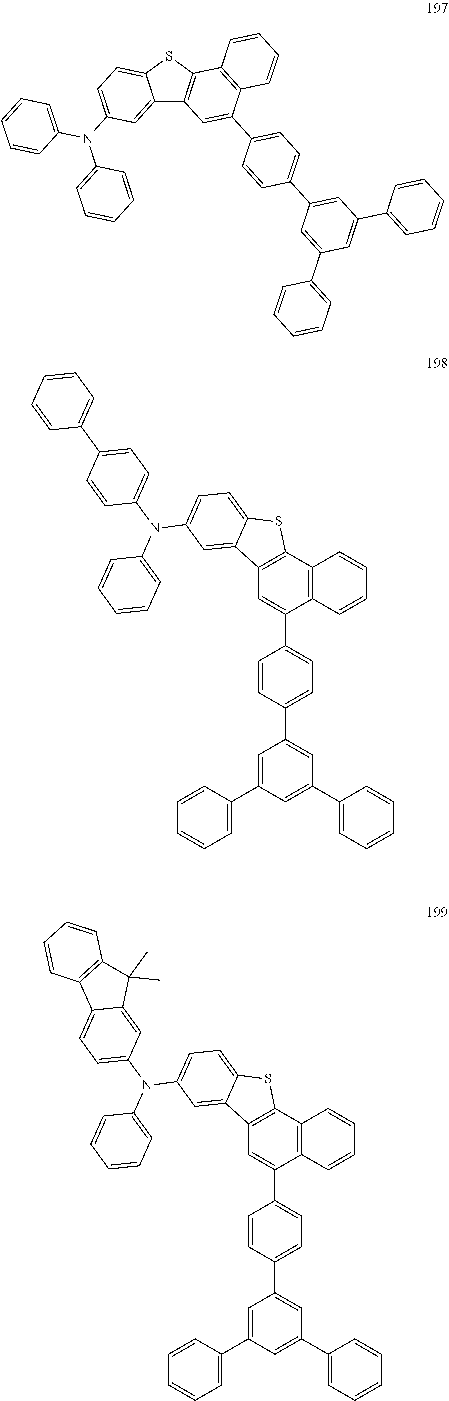 [SMILES: c1ccc(-c2cc(-c3ccccc3)cc(-c3ccc(-c4cc5c6cc(N(c7ccccc7)c7ccccc7)ccc6sc5c5ccccc45)cc3)c2)cc1 ; c1ccc(-c2ccc(N(c3ccccc3)c3ccc4sc5c6ccccc6c(-c6ccc(-c7cc(-c8ccccc8)cc(-c8ccccc8)c7)cc6)cc5c4c3)cc2)cc1 ; CC1(C)c2ccccc2-c2ccc(N(c3ccccc3)c3ccc4sc5c6ccccc6c(-c6ccc(-c7cc(-c8ccccc8)cc(-c8ccccc8)c7)cc6)cc5c4c3)cc21]

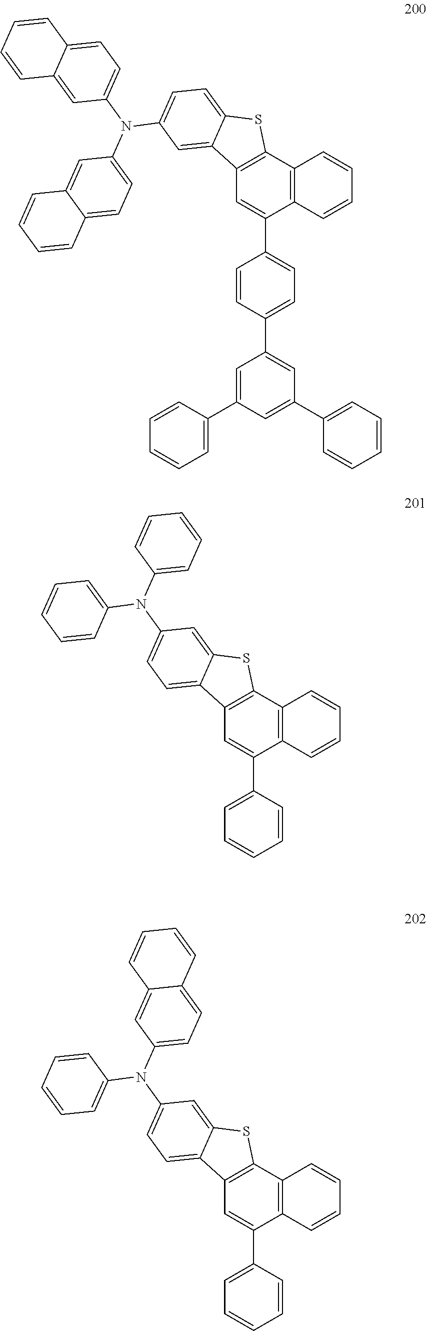

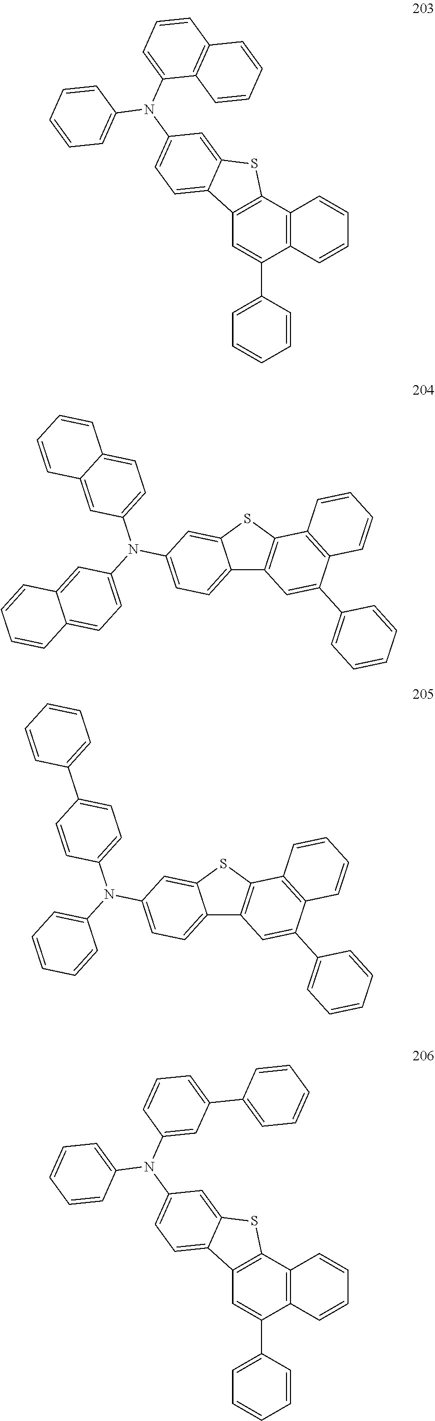 [SMILES: c1ccc(-c2cc3c4ccc(N(c5ccccc5)c5cccc6ccccc56)cc4sc3c3ccccc23)cc1 ; c1ccc(-c2cc3c4ccc(N(c5ccc6ccccc6c5)c5ccc6ccccc6c5)cc4sc3c3ccccc23)cc1 ; c1ccc(-c2ccc(N(c3ccccc3)c3ccc4c(c3)sc3c5ccccc5c(-c5ccccc5)cc43)cc2)cc1 ; c1ccc(-c2cccc(N(c3ccccc3)c3ccc4c(c3)sc3c5ccccc5c(-c5ccccc5)cc43)c2)cc1]

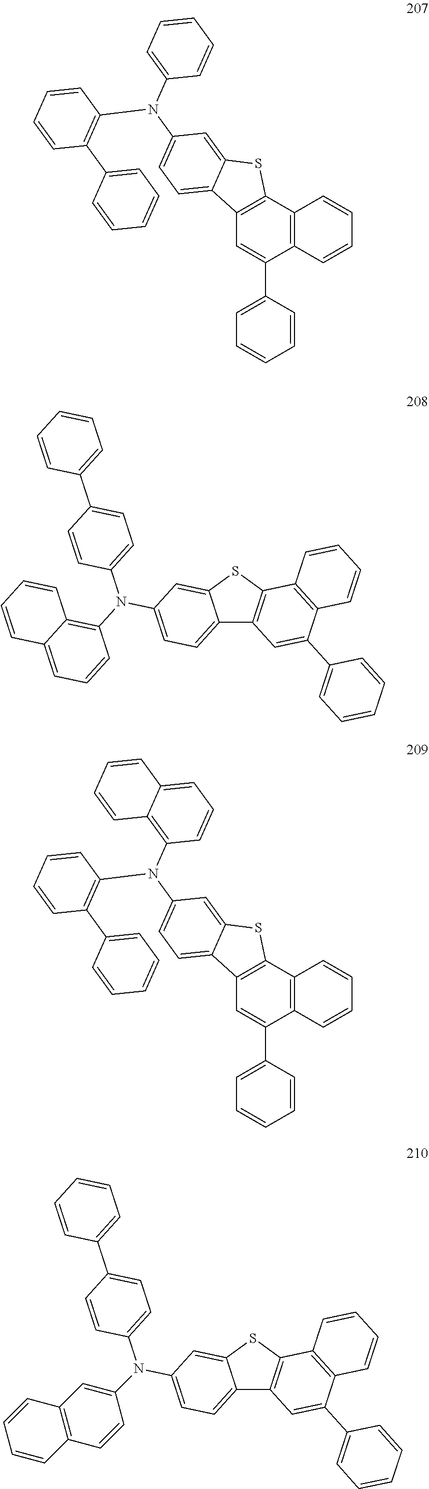 [SMILES: c1ccc(-c2ccccc2N(c2ccccc2)c2ccc3c(c2)sc2c4ccccc4c(-c4ccccc4)cc32)cc1 ; c1ccc(-c2ccc(N(c3ccc4c(c3)sc3c5ccccc5c(-c5ccccc5)cc43)c3cccc4ccccc34)cc2)cc1 ; c1ccc(-c2ccccc2N(c2ccc3c(c2)sc2c4ccccc4c(-c4ccccc4)cc32)c2cccc3ccccc23)cc1 ; c1ccc(-c2ccc(N(c3ccc4ccccc4c3)c3ccc4c(c3)sc3c5ccccc5c(-c5ccccc5)cc43)cc2)cc1]

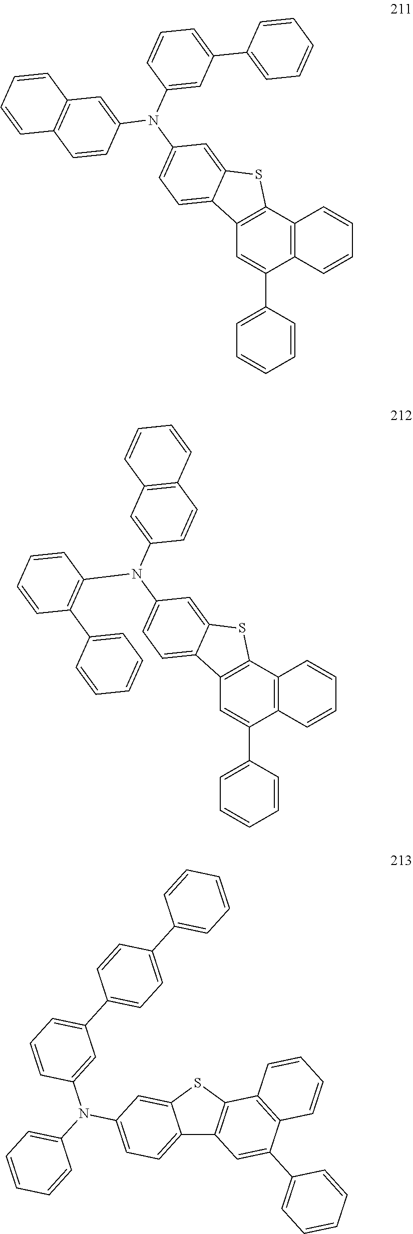 [SMILES: c1ccc(-c2cccc(N(c3ccc4ccccc4c3)c3ccc4c(c3)sc3c5ccccc5c(-c5ccccc5)cc43)c2)cc1 ; c1ccc(-c2ccccc2N(c2ccc3ccccc3c2)c2ccc3c(c2)sc2c4ccccc4c(-c4ccccc4)cc32)cc1 ; c1ccc(-c2ccc(-c3cccc(N(c4ccccc4)c4ccc5c(c4)sc4c6ccccc6c(-c6ccccc6)cc54)c3)cc2)cc1]

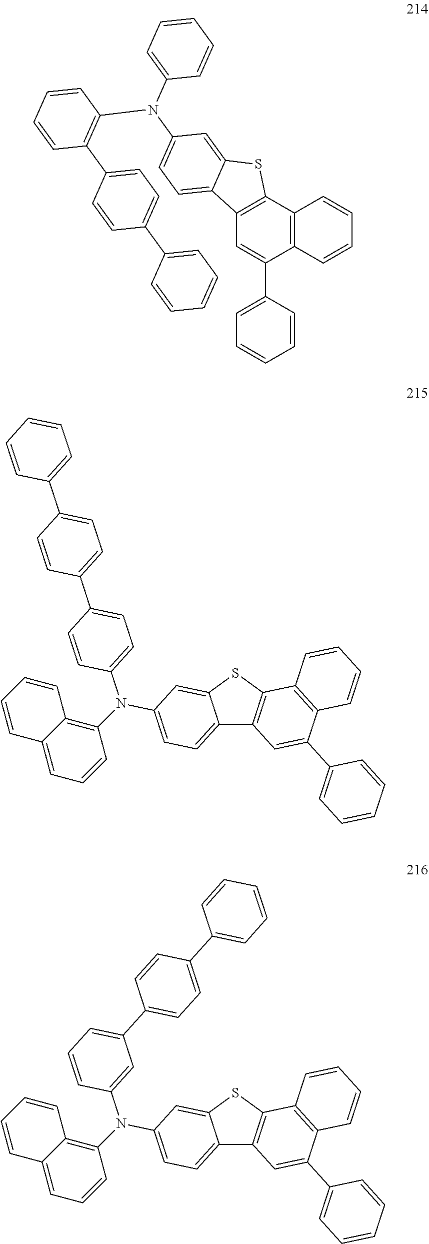 [SMILES: c1ccc(-c2ccc(-c3ccccc3N(c3ccccc3)c3ccc4c(c3)sc3c5ccccc5c(-c5ccccc5)cc43)cc2)cc1 ; c1ccc(-c2ccc(-c3ccc(N(c4ccc5c(c4)sc4c6ccccc6c(-c6ccccc6)cc54)c4cccc5ccccc45)cc3)cc2)cc1 ; c1ccc(-c2ccc(-c3cccc(N(c4ccc5c(c4)sc4c6ccccc6c(-c6ccccc6)cc54)c4cccc5ccccc45)c3)cc2)cc1]

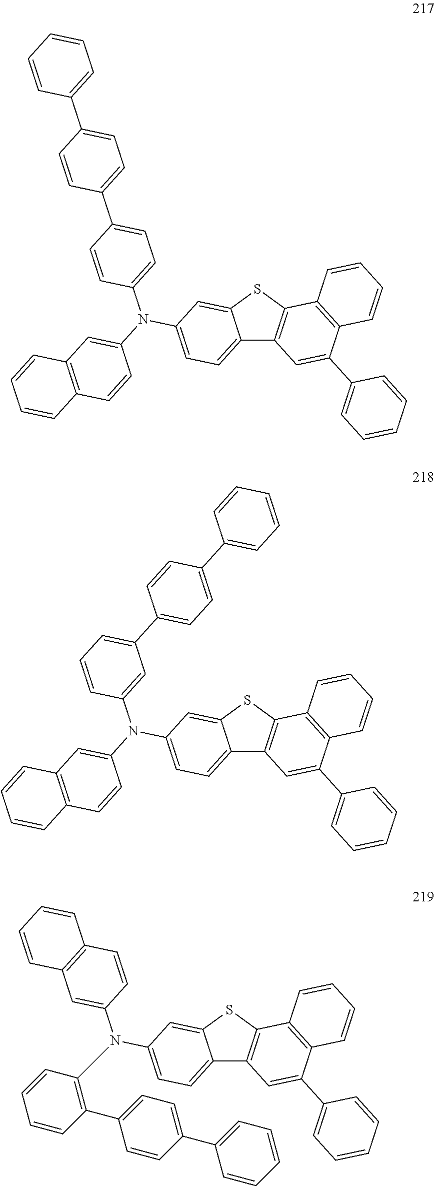 [SMILES: c1ccc(-c2ccc(-c3ccc(N(c4ccc5ccccc5c4)c4ccc5c(c4)sc4c6ccccc6c(-c6ccccc6)cc54)cc3)cc2)cc1 ; c1ccc(-c2ccc(-c3cccc(N(c4ccc5ccccc5c4)c4ccc5c(c4)sc4c6ccccc6c(-c6ccccc6)cc54)c3)cc2)cc1 ; c1ccc(-c2ccc(-c3ccccc3N(c3ccc4ccccc4c3)c3ccc4c(c3)sc3c5ccccc5c(-c5ccccc5)cc43)cc2)cc1]

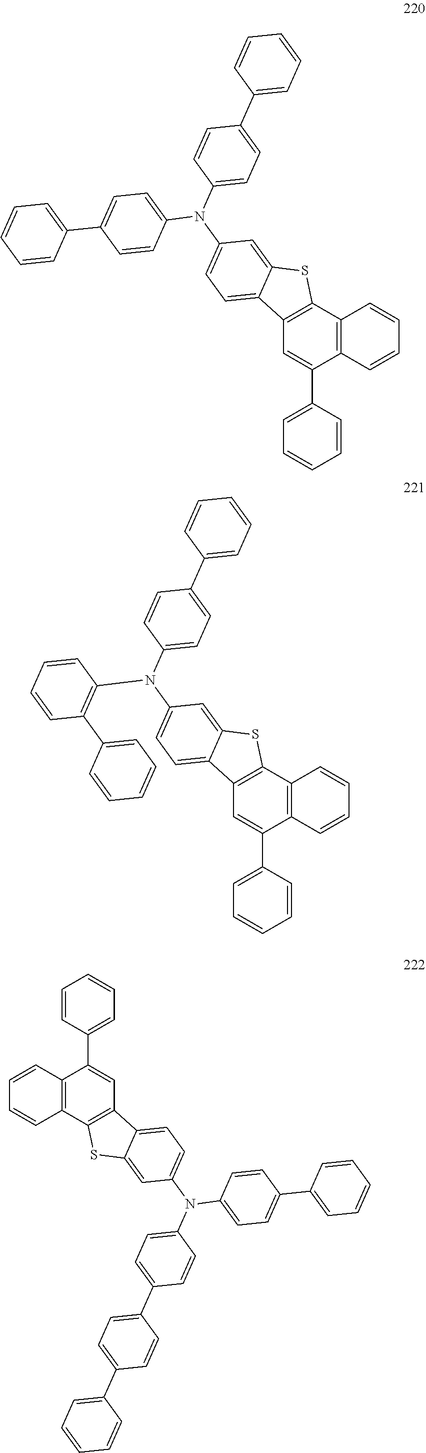 [SMILES: c1ccc(-c2ccc(N(c3ccc(-c4ccccc4)cc3)c3ccc4c(c3)sc3c5ccccc5c(-c5ccccc5)cc43)cc2)cc1 ; c1ccc(-c2ccc(N(c3ccc4c(c3)sc3c5ccccc5c(-c5ccccc5)cc43)c3ccccc3-c3ccccc3)cc2)cc1 ; c1ccc(-c2ccc(-c3ccc(N(c4ccc(-c5ccccc5)cc4)c4ccc5c(c4)sc4c6ccccc6c(-c6ccccc6)cc54)cc3)cc2)cc1]

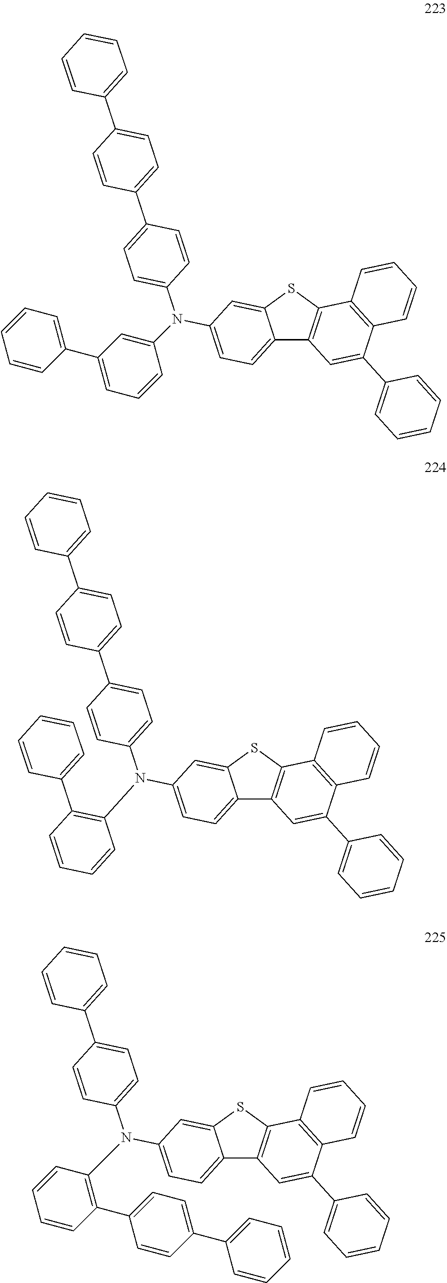 [SMILES: c1ccc(-c2ccc(-c3ccc(N(c4cccc(-c5ccccc5)c4)c4ccc5c(c4)sc4c6ccccc6c(-c6ccccc6)cc54)cc3)cc2)cc1 ; c1ccc(-c2ccc(-c3ccc(N(c4ccc5c(c4)sc4c6ccccc6c(-c6ccccc6)cc54)c4ccccc4-c4ccccc4)cc3)cc2)cc1 ; c1ccc(-c2ccc(-c3ccccc3N(c3ccc(-c4ccccc4)cc3)c3ccc4c(c3)sc3c5ccccc5c(-c5ccccc5)cc43)cc2)cc1]

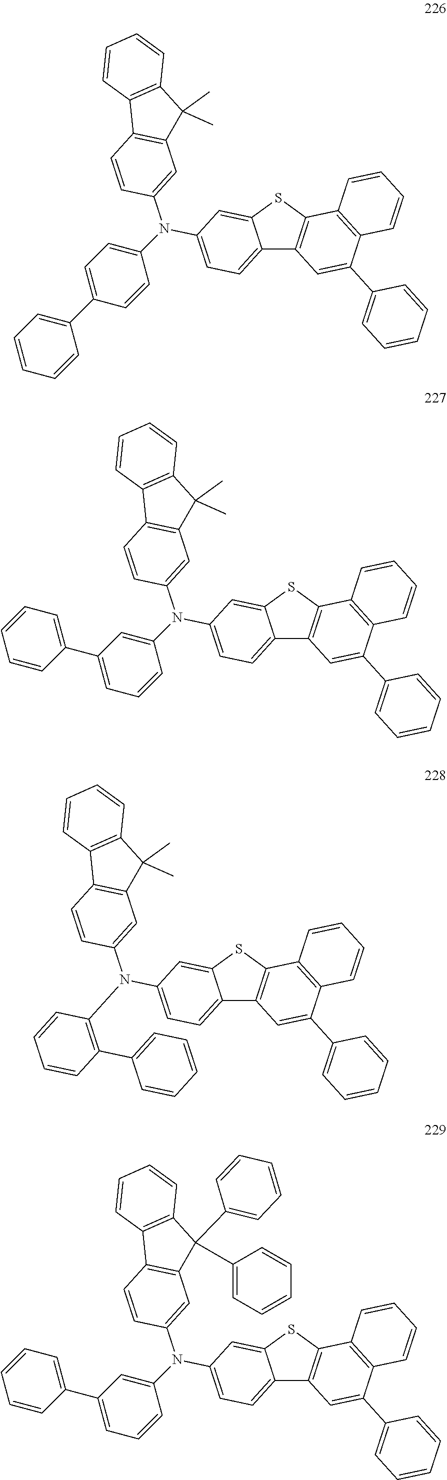 [SMILES: CC1(C)c2ccccc2-c2ccc(N(c3ccc(-c4ccccc4)cc3)c3ccc4c(c3)sc3c5ccccc5c(-c5ccccc5)cc43)cc21 ; CC1(C)c2ccccc2-c2ccc(N(c3cccc(-c4ccccc4)c3)c3ccc4c(c3)sc3c5ccccc5c(-c5ccccc5)cc43)cc21 ; CC1(C)c2ccccc2-c2ccc(N(c3ccc4c(c3)sc3c5ccccc5c(-c5ccccc5)cc43)c3ccccc3-c3ccccc3)cc21 ; c1ccc(-c2cccc(N(c3ccc4c(c3)C(c3ccccc3)(c3ccccc3)c3ccccc3-4)c3ccc4c(c3)sc3c5ccccc5c(-c5ccccc5)cc43)c2)cc1]

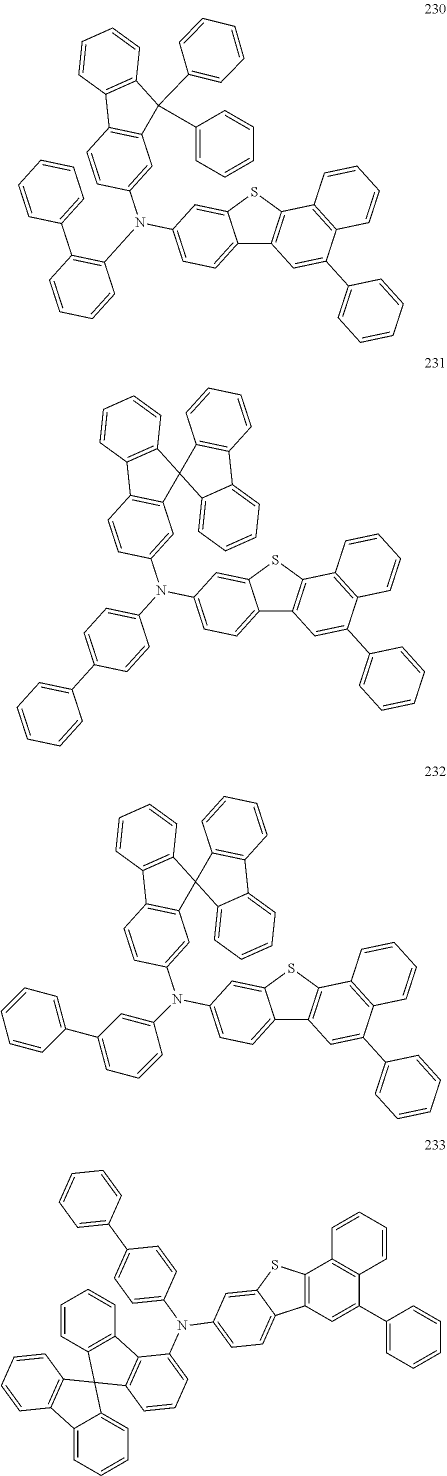 [SMILES: c1ccc(-c2ccccc2N(c2ccc3c(c2)C(c2ccccc2)(c2ccccc2)c2ccccc2-3)c2ccc3c(c2)sc2c4ccccc4c(-c4ccccc4)cc32)cc1 ; c1ccc(-c2ccc(N(c3ccc4c(c3)C3(c5ccccc5-c5ccccc53)c3ccccc3-4)c3ccc4c(c3)sc3c5ccccc5c(-c5ccccc5)cc43)cc2)cc1 ; c1ccc(-c2cccc(N(c3ccc4c(c3)C3(c5ccccc5-c5ccccc53)c3ccccc3-4)c3ccc4c(c3)sc3c5ccccc5c(-c5ccccc5)cc43)c2)cc1 ; c1ccc(-c2ccc(N(c3ccc4c(c3)sc3c5ccccc5c(-c5ccccc5)cc43)c3cccc4c3-c3ccccc3C43c4ccccc4-c4ccccc43)cc2)cc1]

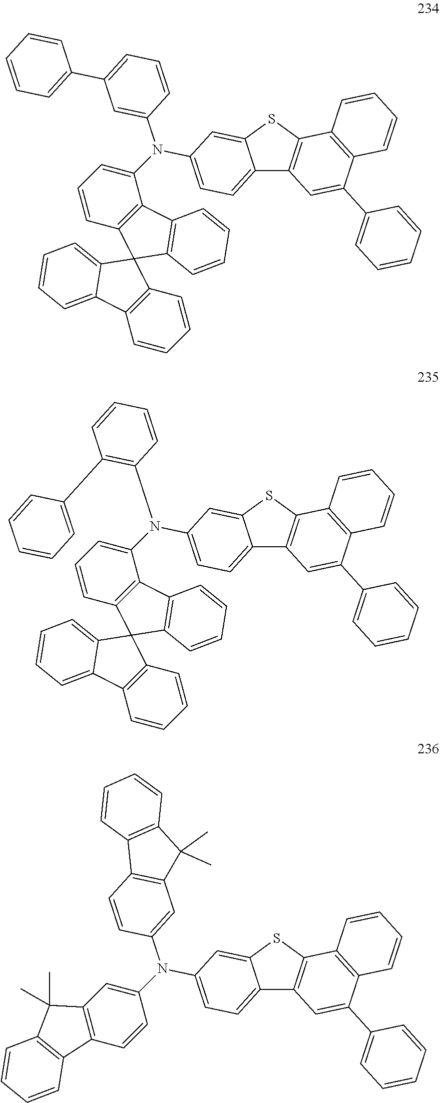 [SMILES: c1ccc(-c2cccc(N(c3ccc4c(c3)sc3c5ccccc5c(-c5ccccc5)cc43)c3cccc4c3-c3ccccc3C43c4ccccc4-c4ccccc43)c2)cc1 ; c1ccc(-c2ccccc2N(c2ccc3c(c2)sc2c4ccccc4c(-c4ccccc4)cc32)c2cccc3c2-c2ccccc2C32c3ccccc3-c3ccccc32)cc1 ; CC1(C)c2ccccc2-c2ccc(N(c3ccc4c(c3)C(C)(C)c3ccccc3-4)c3ccc4c(c3)sc3c5ccccc5c(-c5ccccc5)cc43)cc21]

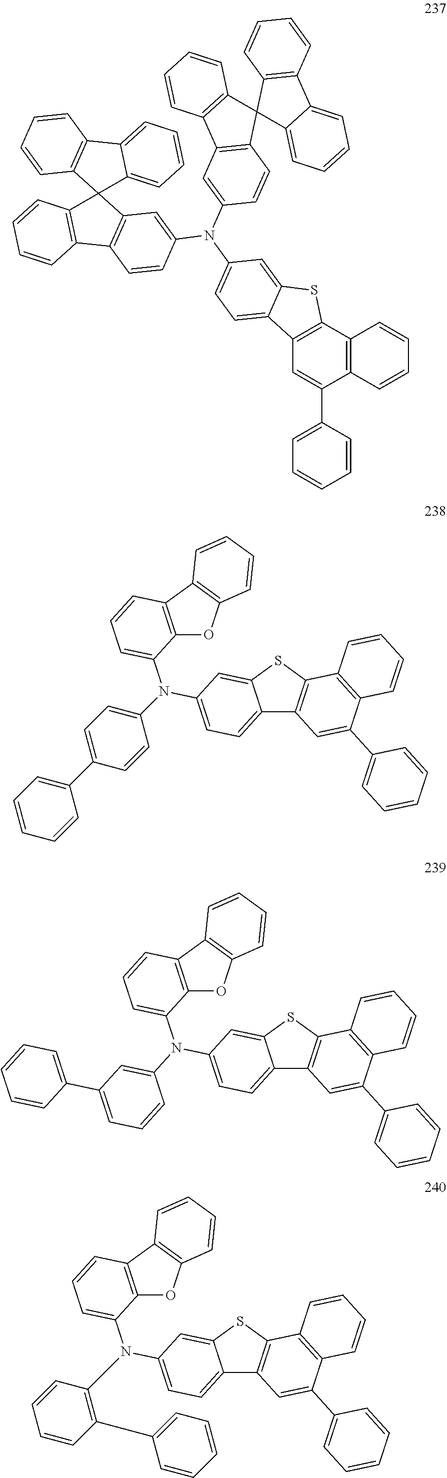 [SMILES: c1ccc(-c2cc3c4ccc(N(c5ccc6c(c5)-c5ccccc5C65c6ccccc6-c6ccccc65)c5ccc6c(c5)C5(c7ccccc7-c7ccccc75)c5ccccc5-6)cc4sc3c3ccccc23)cc1 ; c1ccc(-c2ccc(N(c3ccc4c(c3)sc3c5ccccc5c(-c5ccccc5)cc43)c3cccc4c3oc3ccccc34)cc2)cc1 ; c1ccc(-c2cccc(N(c3ccc4c(c3)sc3c5ccccc5c(-c5ccccc5)cc43)c3cccc4c3oc3ccccc34)c2)cc1 ; c1ccc(-c2ccccc2N(c2ccc3c(c2)sc2c4ccccc4c(-c4ccccc4)cc32)c2cccc3c2oc2ccccc23)cc1]

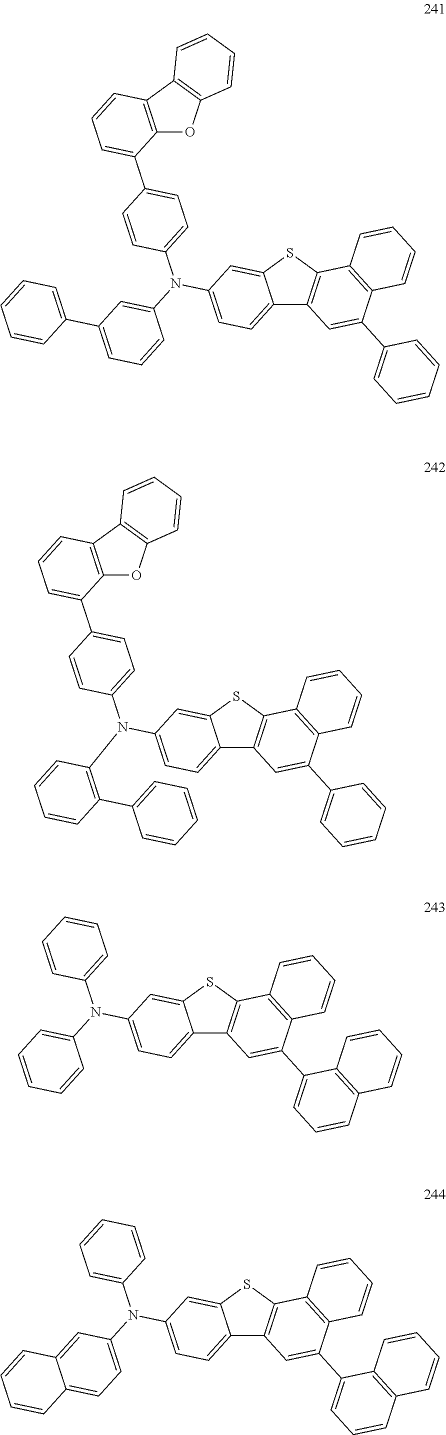 [SMILES: c1ccc(-c2cccc(N(c3ccc(-c4cccc5c4oc4ccccc45)cc3)c3ccc4c(c3)sc3c5ccccc5c(-c5ccccc5)cc43)c2)cc1 ; c1ccc(-c2ccccc2N(c2ccc(-c3cccc4c3oc3ccccc34)cc2)c2ccc3c(c2)sc2c4ccccc4c(-c4ccccc4)cc32)cc1 ; c1ccc(N(c2ccccc2)c2ccc3c(c2)sc2c4ccccc4c(-c4cccc5ccccc45)cc32)cc1 ; c1ccc(N(c2ccc3ccccc3c2)c2ccc3c(c2)sc2c4ccccc4c(-c4cccc5ccccc45)cc32)cc1]

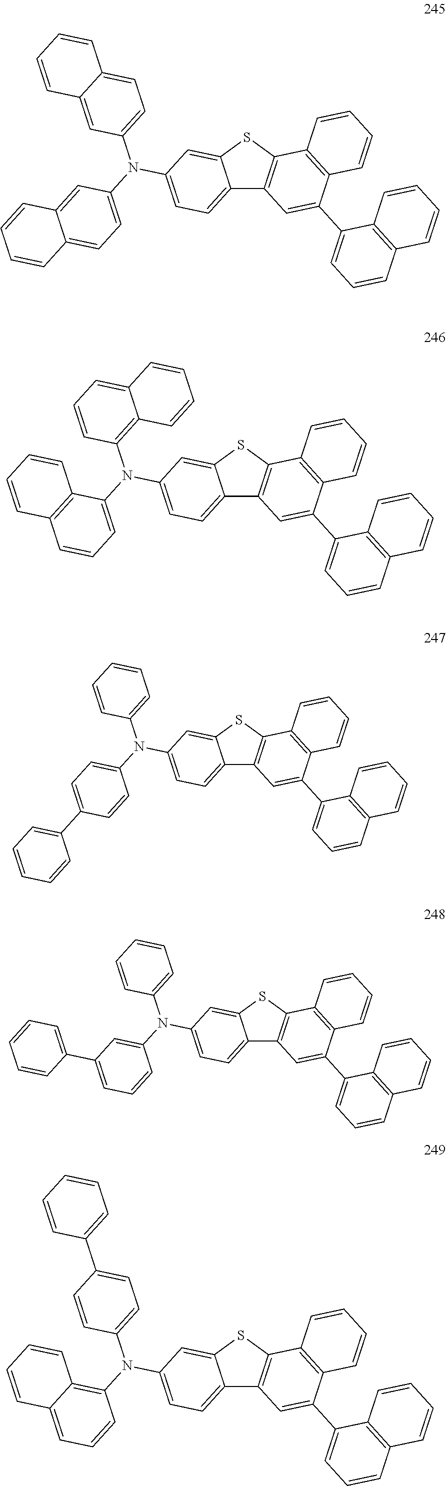 [SMILES: c1ccc2cc(N(c3ccc4ccccc4c3)c3ccc4c(c3)sc3c5ccccc5c(-c5cccc6ccccc56)cc43)ccc2c1 ; c1ccc2c(-c3cc4c5ccc(N(c6cccc7ccccc67)c6cccc7ccccc67)cc5sc4c4ccccc34)cccc2c1 ; c1ccc(-c2ccc(N(c3ccccc3)c3ccc4c(c3)sc3c5ccccc5c(-c5cccc6ccccc56)cc43)cc2)cc1 ; c1ccc(-c2cccc(N(c3ccccc3)c3ccc4c(c3)sc3c5ccccc5c(-c5cccc6ccccc56)cc43)c2)cc1 ; c1ccc(-c2ccc(N(c3ccc4c(c3)sc3c5ccccc5c(-c5cccc6ccccc56)cc43)c3cccc4ccccc34)cc2)cc1]

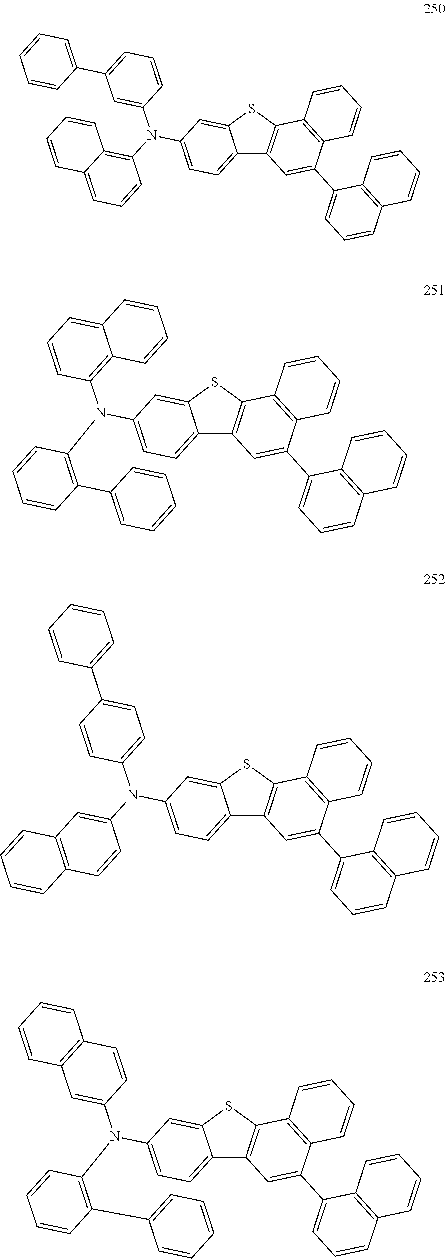 [SMILES: c1ccc(-c2cccc(N(c3ccc4c(c3)sc3c5ccccc5c(-c5cccc6ccccc56)cc43)c3cccc4ccccc34)c2)cc1 ; c1ccc(-c2ccccc2N(c2ccc3c(c2)sc2c4ccccc4c(-c4cccc5ccccc45)cc32)c2cccc3ccccc23)cc1 ; c1ccc(-c2ccc(N(c3ccc4ccccc4c3)c3ccc4c(c3)sc3c5ccccc5c(-c5cccc6ccccc56)cc43)cc2)cc1 ; c1ccc(-c2ccccc2N(c2ccc3ccccc3c2)c2ccc3c(c2)sc2c4ccccc4c(-c4cccc5ccccc45)cc32)cc1]

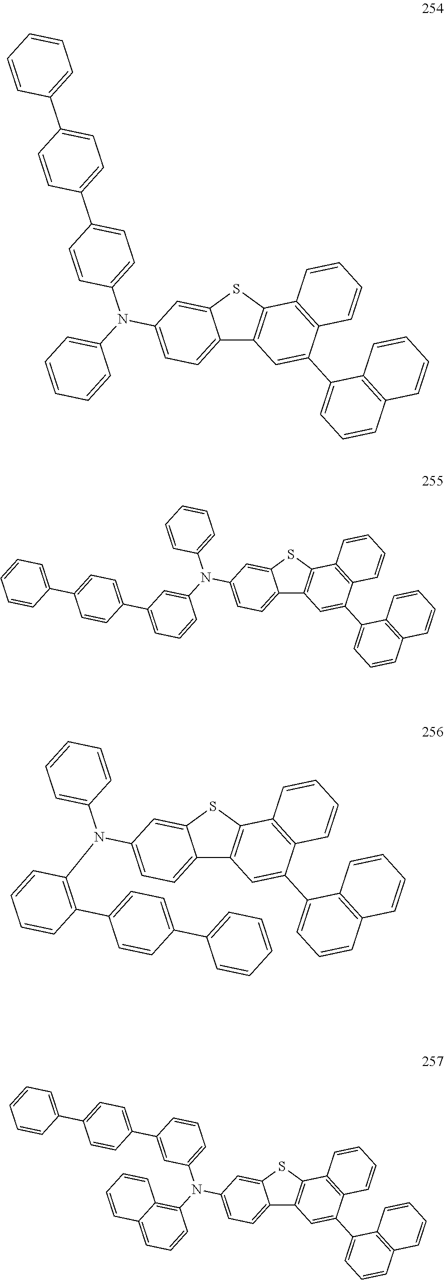 [SMILES: c1ccc(-c2ccc(-c3ccc(N(c4ccccc4)c4ccc5c(c4)sc4c6ccccc6c(-c6cccc7ccccc67)cc54)cc3)cc2)cc1 ; c1ccc(-c2ccc(-c3cccc(N(c4ccccc4)c4ccc5c(c4)sc4c6ccccc6c(-c6cccc7ccccc67)cc54)c3)cc2)cc1 ; c1ccc(-c2ccc(-c3ccccc3N(c3ccccc3)c3ccc4c(c3)sc3c5ccccc5c(-c5cccc6ccccc56)cc43)cc2)cc1 ; c1ccc(-c2ccc(-c3cccc(N(c4ccc5c(c4)sc4c6ccccc6c(-c6cccc7ccccc67)cc54)c4cccc5ccccc45)c3)cc2)cc1]

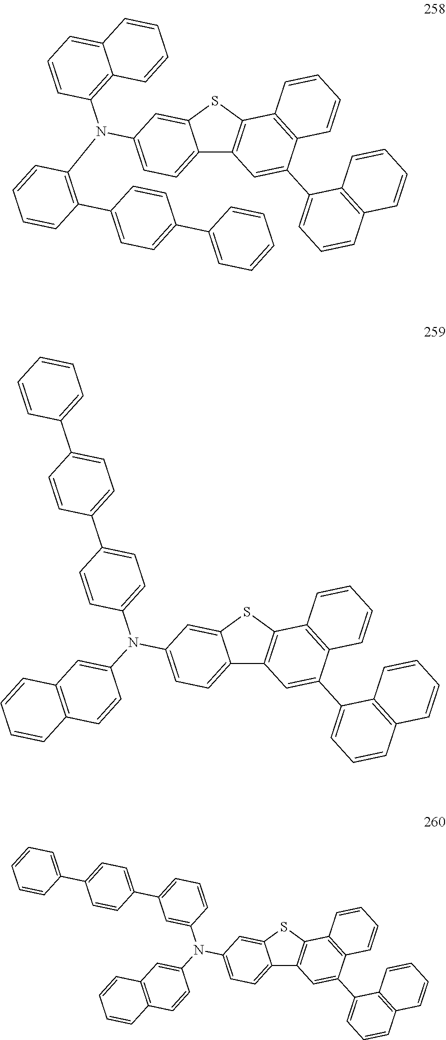 [SMILES: c1ccc(-c2ccc(-c3ccccc3N(c3ccc4c(c3)sc3c5ccccc5c(-c5cccc6ccccc56)cc43)c3cccc4ccccc34)cc2)cc1 ; c1ccc(-c2ccc(-c3ccc(N(c4ccc5ccccc5c4)c4ccc5c(c4)sc4c6ccccc6c(-c6cccc7ccccc67)cc54)cc3)cc2)cc1 ; c1ccc(-c2ccc(-c3cccc(N(c4ccc5ccccc5c4)c4ccc5c(c4)sc4c6ccccc6c(-c6cccc7ccccc67)cc54)c3)cc2)cc1]

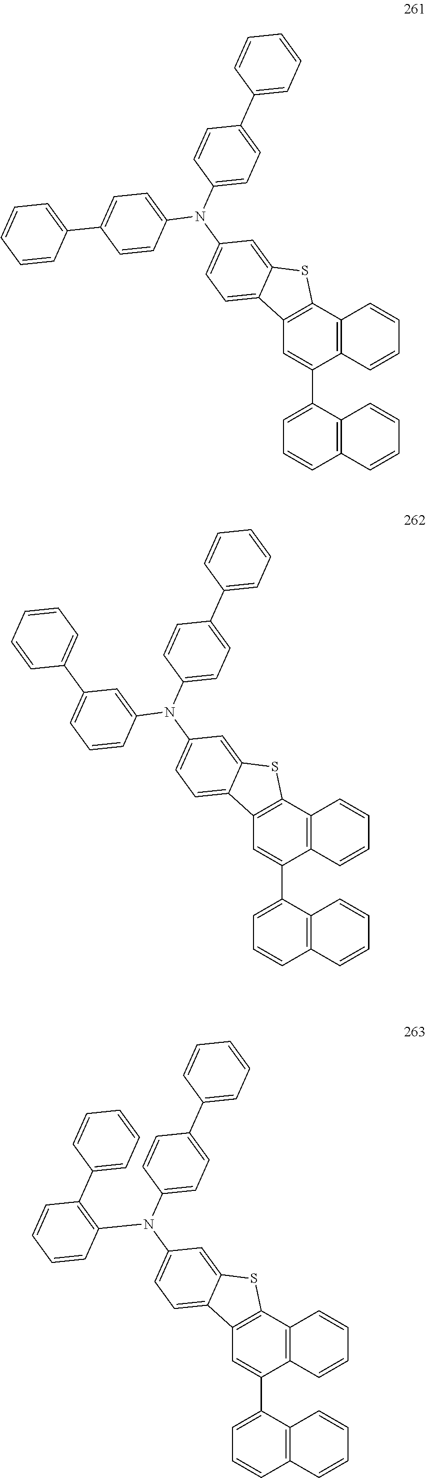 [SMILES: c1ccc(-c2ccc(N(c3ccc(-c4ccccc4)cc3)c3ccc4c(c3)sc3c5ccccc5c(-c5cccc6ccccc56)cc43)cc2)cc1 ; c1ccc(-c2ccc(N(c3cccc(-c4ccccc4)c3)c3ccc4c(c3)sc3c5ccccc5c(-c5cccc6ccccc56)cc43)cc2)cc1 ; c1ccc(-c2ccc(N(c3ccc4c(c3)sc3c5ccccc5c(-c5cccc6ccccc56)cc43)c3ccccc3-c3ccccc3)cc2)cc1]

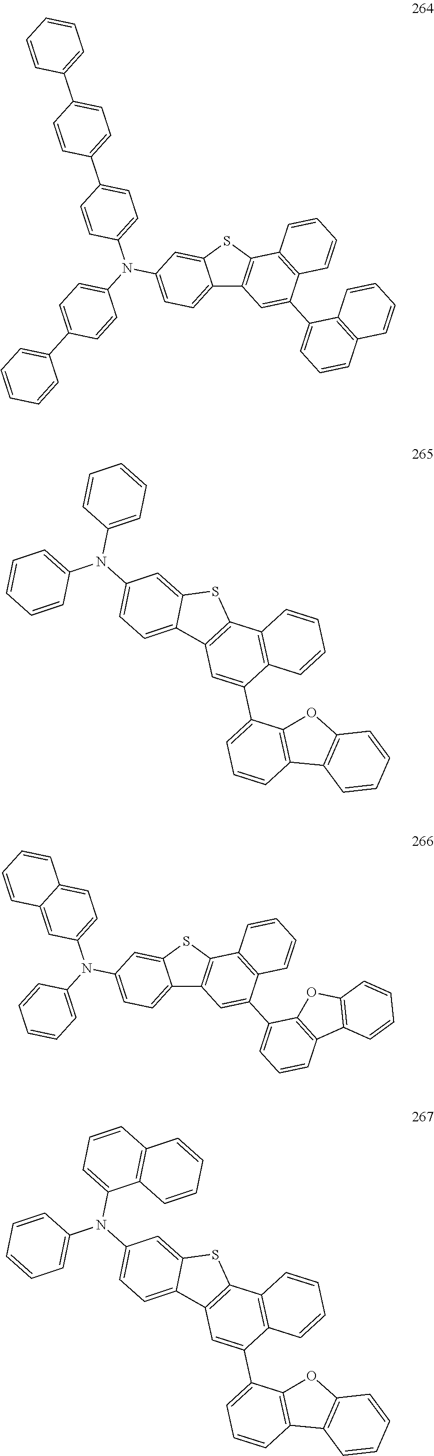 [SMILES: c1ccc(-c2ccc(-c3ccc(N(c4ccc(-c5ccccc5)cc4)c4ccc5c(c4)sc4c6ccccc6c(-c6cccc7ccccc67)cc54)cc3)cc2)cc1 ; c1ccc(N(c2ccccc2)c2ccc3c(c2)sc2c4ccccc4c(-c4cccc5c4oc4ccccc45)cc32)cc1 ; c1ccc(N(c2ccc3ccccc3c2)c2ccc3c(c2)sc2c4ccccc4c(-c4cccc5c4oc4ccccc45)cc32)cc1 ; c1ccc(N(c2ccc3c(c2)sc2c4ccccc4c(-c4cccc5c4oc4ccccc45)cc32)c2cccc3ccccc23)cc1]

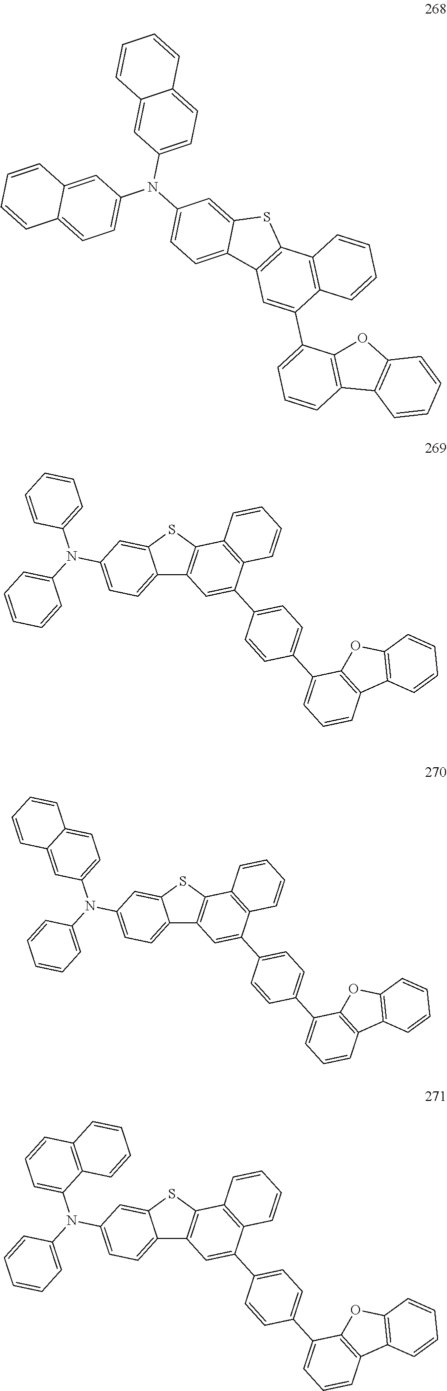 [SMILES: c1ccc2cc(N(c3ccc4ccccc4c3)c3ccc4c(c3)sc3c5ccccc5c(-c5cccc6c5oc5ccccc56)cc43)ccc2c1 ; c1ccc(N(c2ccccc2)c2ccc3c(c2)sc2c4ccccc4c(-c4ccc(-c5cccc6c5oc5ccccc56)cc4)cc32)cc1 ; c1ccc(N(c2ccc3ccccc3c2)c2ccc3c(c2)sc2c4ccccc4c(-c4ccc(-c5cccc6c5oc5ccccc56)cc4)cc32)cc1 ; c1ccc(N(c2ccc3c(c2)sc2c4ccccc4c(-c4ccc(-c5cccc6c5oc5ccccc56)cc4)cc32)c2cccc3ccccc23)cc1]

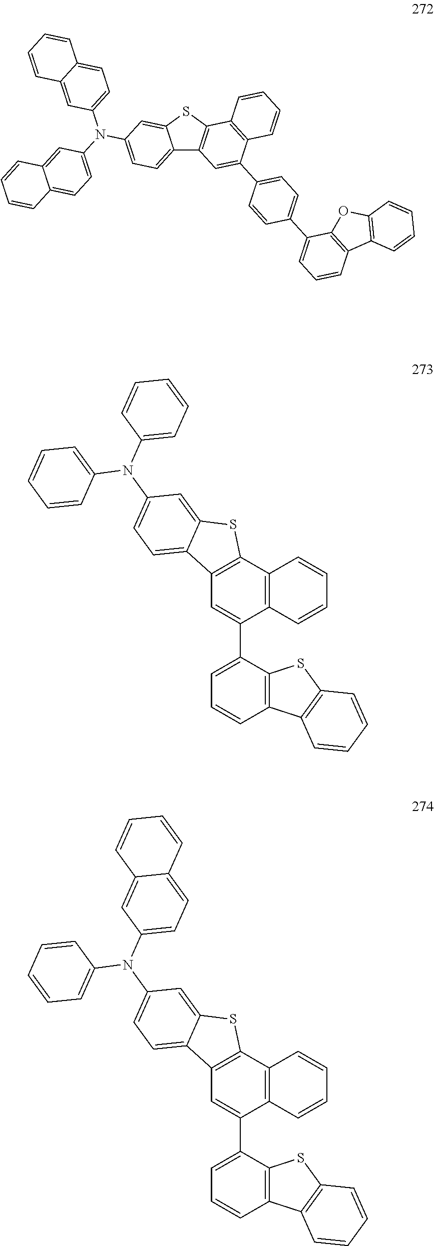 [SMILES: c1ccc2cc(N(c3ccc4ccccc4c3)c3ccc4c(c3)sc3c5ccccc5c(-c5ccc(-c6cccc7c6oc6ccccc67)cc5)cc43)ccc2c1 ; c1ccc(N(c2ccccc2)c2ccc3c(c2)sc2c4ccccc4c(-c4cccc5c4sc4ccccc45)cc32)cc1 ; c1ccc(N(c2ccc3ccccc3c2)c2ccc3c(c2)sc2c4ccccc4c(-c4cccc5c4sc4ccccc45)cc32)cc1]

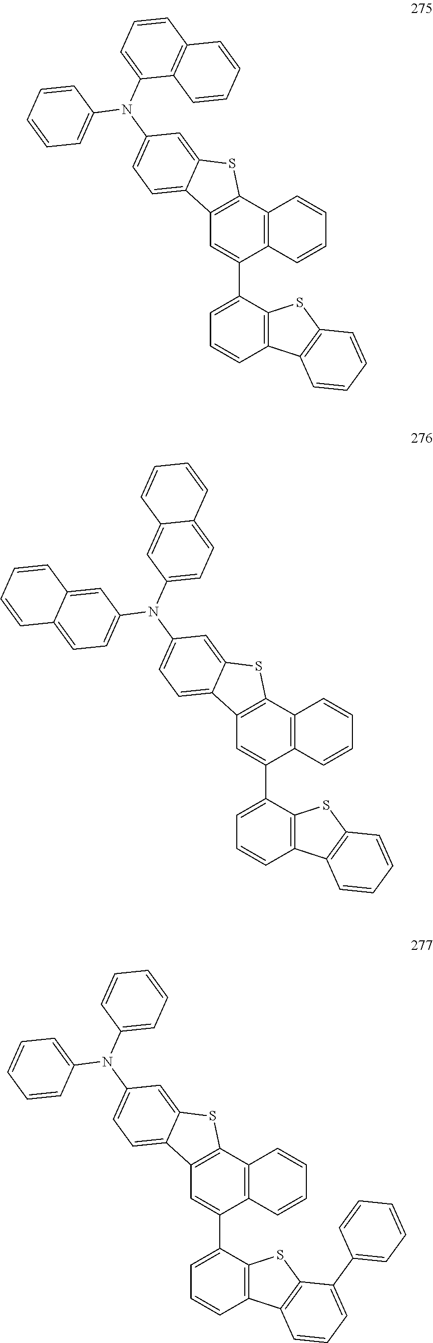 [SMILES: c1ccc(N(c2ccc3c(c2)sc2c4ccccc4c(-c4cccc5c4sc4ccccc45)cc32)c2cccc3ccccc23)cc1 ; c1ccc2cc(N(c3ccc4ccccc4c3)c3ccc4c(c3)sc3c5ccccc5c(-c5cccc6c5sc5ccccc56)cc43)ccc2c1 ; c1ccc(-c2cccc3c2sc2c(-c4cc5c6ccc(N(c7ccccc7)c7ccccc7)cc6sc5c5ccccc45)cccc23)cc1]

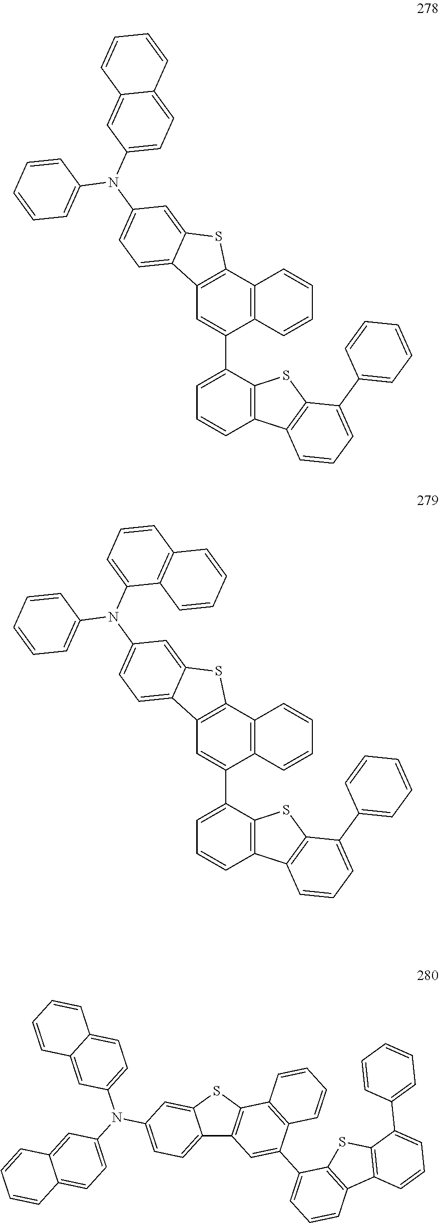 [SMILES: c1ccc(-c2cccc3c2sc2c(-c4cc5c6ccc(N(c7ccccc7)c7ccc8ccccc8c7)cc6sc5c5ccccc45)cccc23)cc1 ; c1ccc(-c2cccc3c2sc2c(-c4cc5c6ccc(N(c7ccccc7)c7cccc8ccccc78)cc6sc5c5ccccc45)cccc23)cc1 ; c1ccc(-c2cccc3c2sc2c(-c4cc5c6ccc(N(c7ccc8ccccc8c7)c7ccc8ccccc8c7)cc6sc5c5ccccc45)cccc23)cc1]

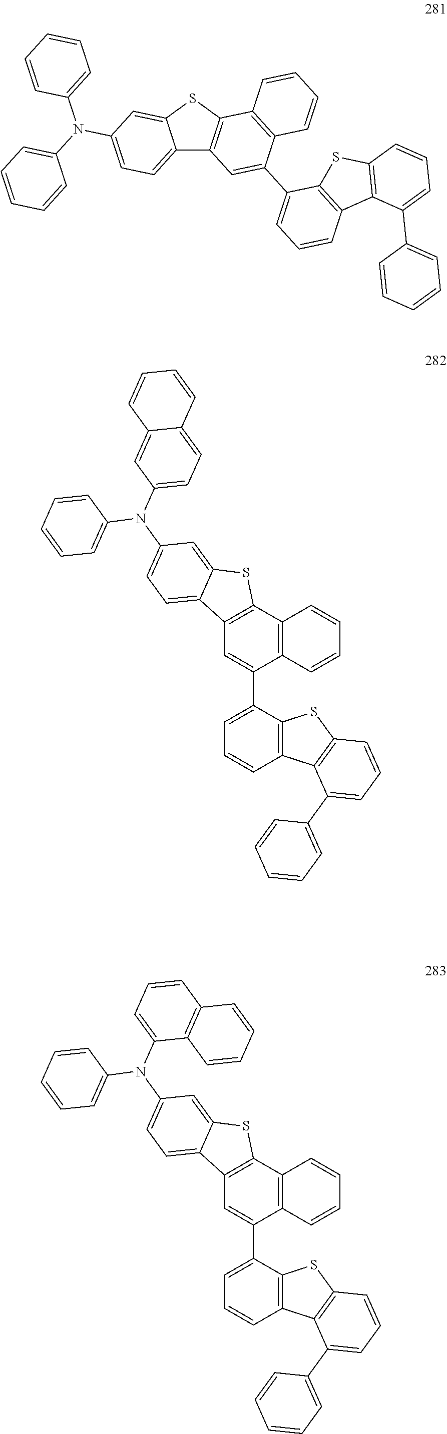 [SMILES: c1ccc(-c2cccc3sc4c(-c5cc6c7ccc(N(c8ccccc8)c8ccccc8)cc7sc6c6ccccc56)cccc4c23)cc1 ; c1ccc(-c2cccc3sc4c(-c5cc6c7ccc(N(c8ccccc8)c8ccc9ccccc9c8)cc7sc6c6ccccc56)cccc4c23)cc1 ; c1ccc(-c2cccc3sc4c(-c5cc6c7ccc(N(c8ccccc8)c8cccc9ccccc89)cc7sc6c6ccccc56)cccc4c23)cc1]

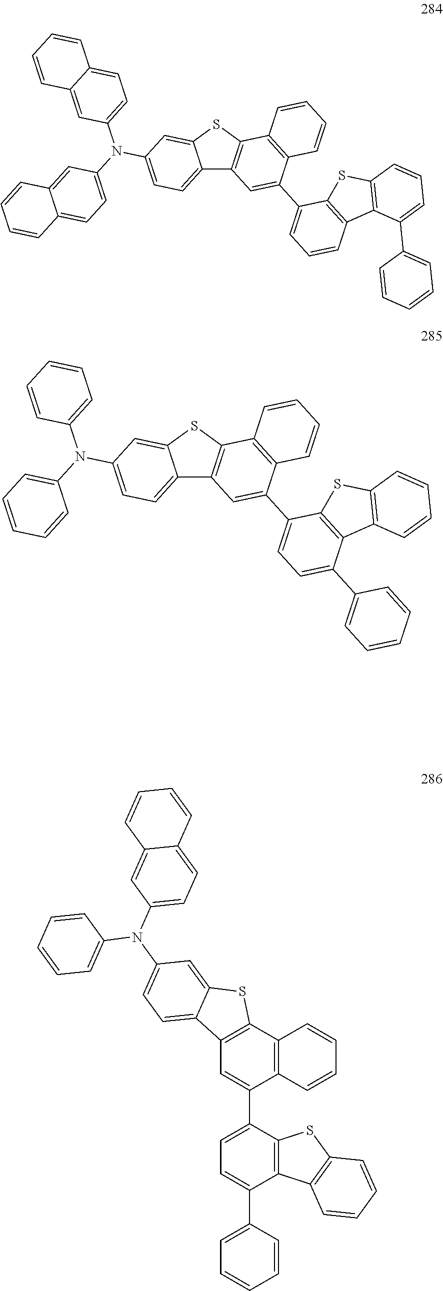 [SMILES: c1ccc(-c2cccc3sc4c(-c5cc6c7ccc(N(c8ccc9ccccc9c8)c8ccc9ccccc9c8)cc7sc6c6ccccc56)cccc4c23)cc1 ; c1ccc(-c2ccc(-c3cc4c5ccc(N(c6ccccc6)c6ccccc6)cc5sc4c4ccccc34)c3sc4ccccc4c23)cc1 ; c1ccc(-c2ccc(-c3cc4c5ccc(N(c6ccccc6)c6ccc7ccccc7c6)cc5sc4c4ccccc34)c3sc4ccccc4c23)cc1]

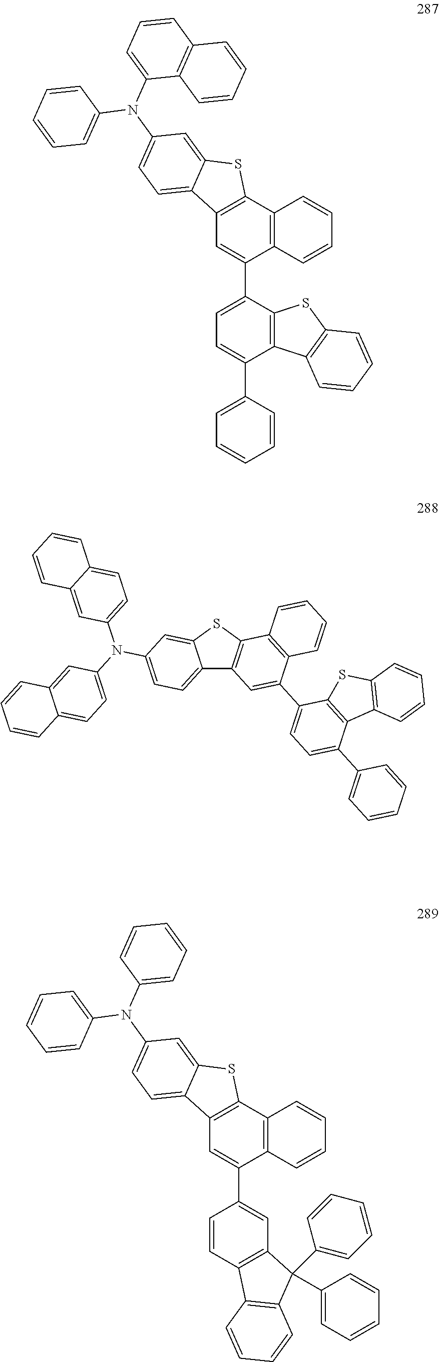 [SMILES: c1ccc(-c2ccc(-c3cc4c5ccc(N(c6ccccc6)c6cccc7ccccc67)cc5sc4c4ccccc34)c3sc4ccccc4c23)cc1 ; c1ccc(-c2ccc(-c3cc4c5ccc(N(c6ccc7ccccc7c6)c6ccc7ccccc7c6)cc5sc4c4ccccc34)c3sc4ccccc4c23)cc1 ; c1ccc(N(c2ccccc2)c2ccc3c(c2)sc2c4ccccc4c(-c4ccc5c(c4)C(c4ccccc4)(c4ccccc4)c4ccccc4-5)cc32)cc1]

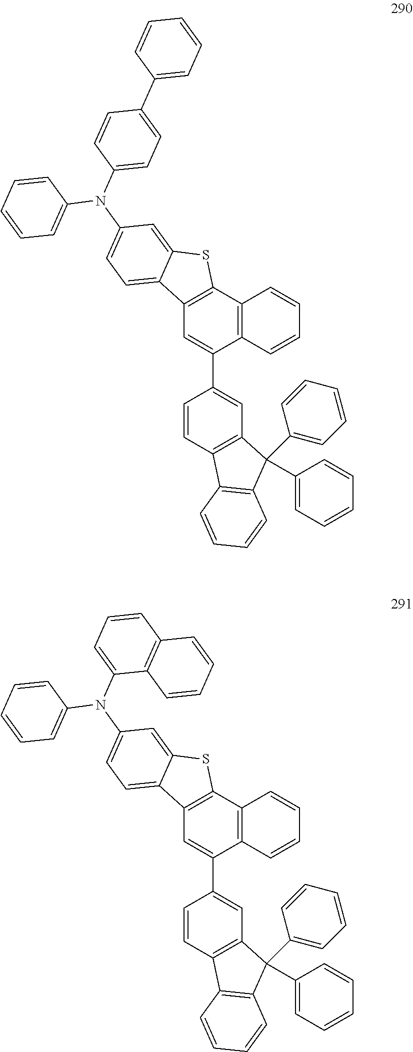 [SMILES: c1ccc(-c2ccc(N(c3ccccc3)c3ccc4c(c3)sc3c5ccccc5c(-c5ccc6c(c5)C(c5ccccc5)(c5ccccc5)c5ccccc5-6)cc43)cc2)cc1 ; c1ccc(N(c2ccc3c(c2)sc2c4ccccc4c(-c4ccc5c(c4)C(c4ccccc4)(c4ccccc4)c4ccccc4-5)cc32)c2cccc3ccccc23)cc1]

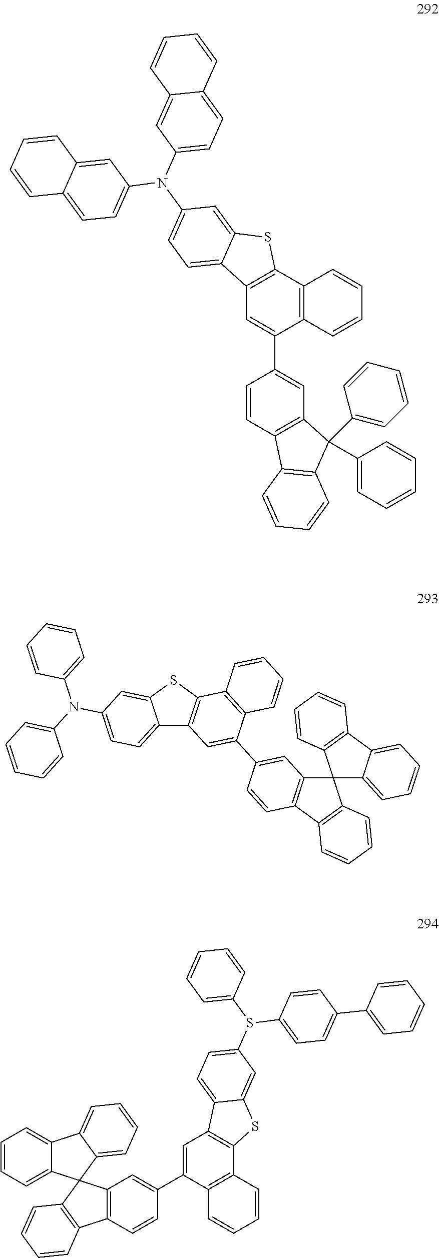

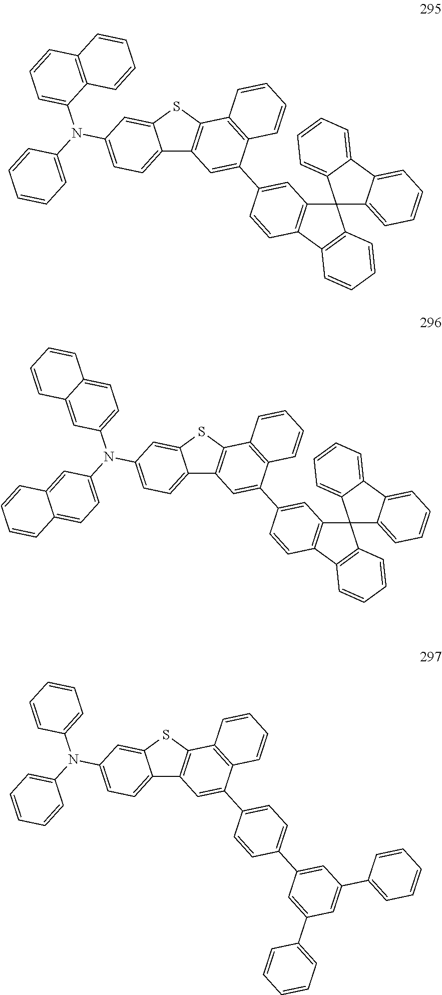 [SMILES: c1ccc(N(c2ccc3c(c2)sc2c4ccccc4c(-c4ccc5c(c4)C4(c6ccccc6-c6ccccc64)c4ccccc4-5)cc32)c2cccc3ccccc23)cc1 ; c1ccc2c(c1)-c1ccccc1C21c2ccccc2-c2ccc(-c3cc4c5ccc(N(c6ccc7ccccc7c6)c6ccc7ccccc7c6)cc5sc4c4ccccc34)cc21 ; c1ccc(-c2cc(-c3ccccc3)cc(-c3ccc(-c4cc5c6ccc(N(c7ccccc7)c7ccccc7)cc6sc5c5ccccc45)cc3)c2)cc1]

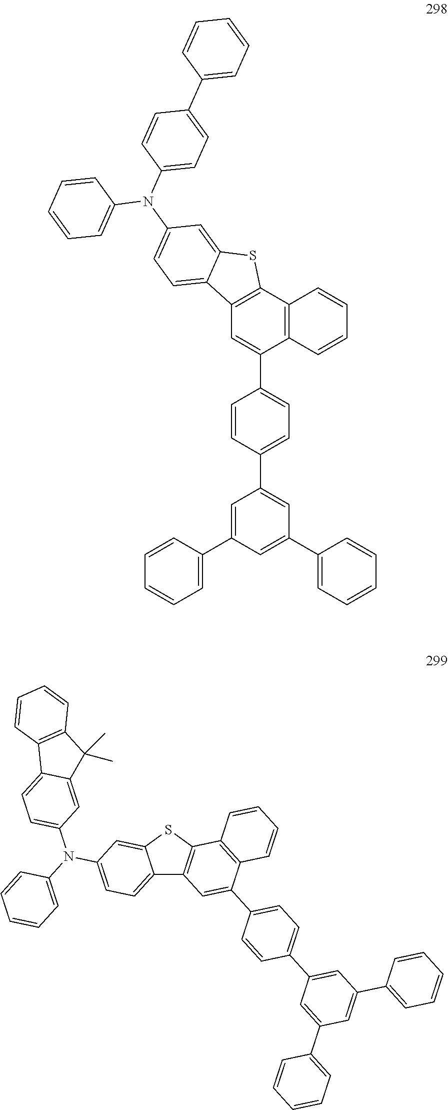 [SMILES: c1ccc(-c2ccc(N(c3ccccc3)c3ccc4c(c3)sc3c5ccccc5c(-c5ccc(-c6cc(-c7ccccc7)cc(-c7ccccc7)c6)cc5)cc43)cc2)cc1 ; CC1(C)c2ccccc2-c2ccc(N(c3ccccc3)c3ccc4c(c3)sc3c5ccccc5c(-c5ccc(-c6cc(-c7ccccc7)cc(-c7ccccc7)c6)cc5)cc43)cc21]

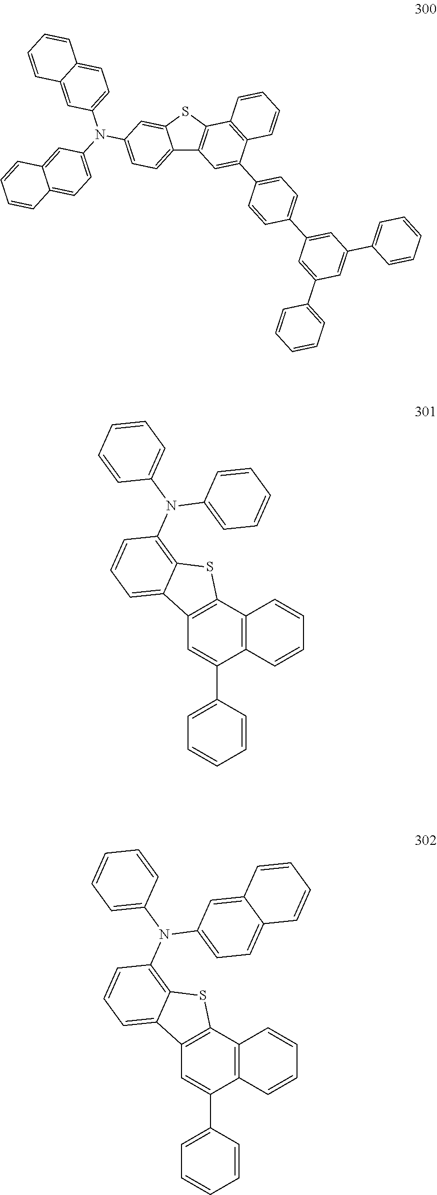 [SMILES: c1ccc(-c2cc(-c3ccccc3)cc(-c3ccc(-c4cc5c6ccc(N(c7ccc8ccccc8c7)c7ccc8ccccc8c7)cc6sc5c5ccccc45)cc3)c2)cc1 ; c1ccc(-c2cc3c4cccc(N(c5ccccc5)c5ccccc5)c4sc3c3ccccc23)cc1 ; c1ccc(-c2cc3c4cccc(N(c5ccccc5)c5ccc6ccccc6c5)c4sc3c3ccccc23)cc1]

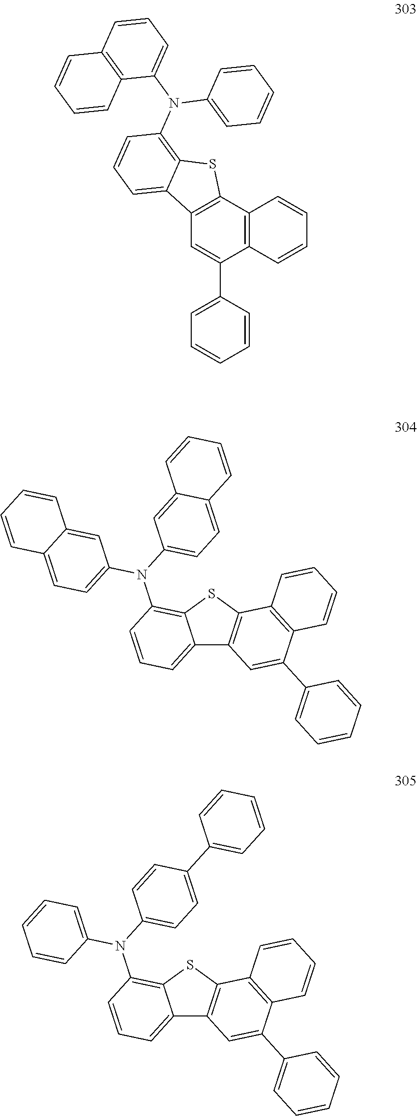 [SMILES: c1ccc(-c2cc3c4cccc(N(c5ccccc5)c5cccc6ccccc56)c4sc3c3ccccc23)cc1 ; c1ccc(-c2cc3c4cccc(N(c5ccc6ccccc6c5)c5ccc6ccccc6c5)c4sc3c3ccccc23)cc1 ; c1ccc(-c2ccc(N(c3ccccc3)c3cccc4c3sc3c5ccccc5c(-c5ccccc5)cc43)cc2)cc1]

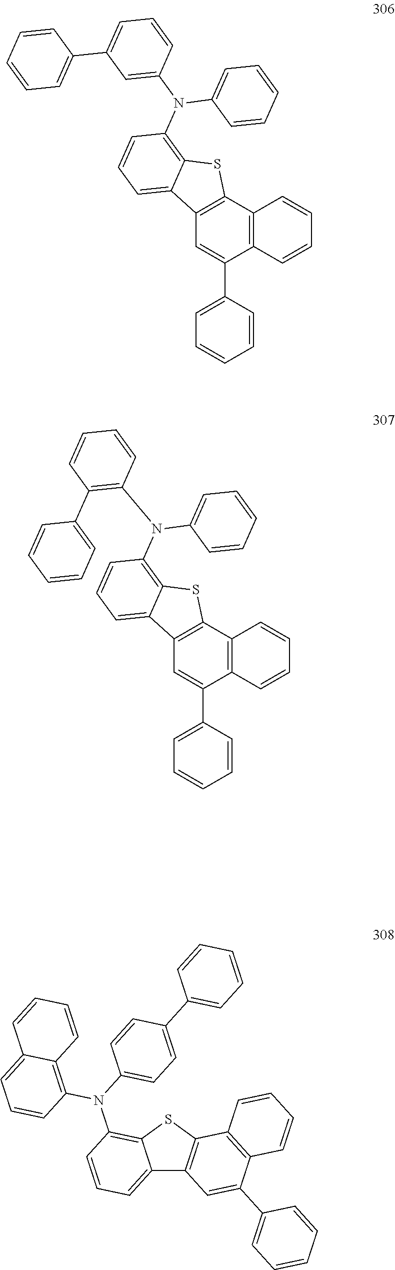 [SMILES: c1ccc(-c2cccc(N(c3ccccc3)c3cccc4c3sc3c5ccccc5c(-c5ccccc5)cc43)c2)cc1 ; c1ccc(-c2ccccc2N(c2ccccc2)c2cccc3c2sc2c4ccccc4c(-c4ccccc4)cc32)cc1 ; c1ccc(-c2ccc(N(c3cccc4ccccc34)c3cccc4c3sc3c5ccccc5c(-c5ccccc5)cc43)cc2)cc1]

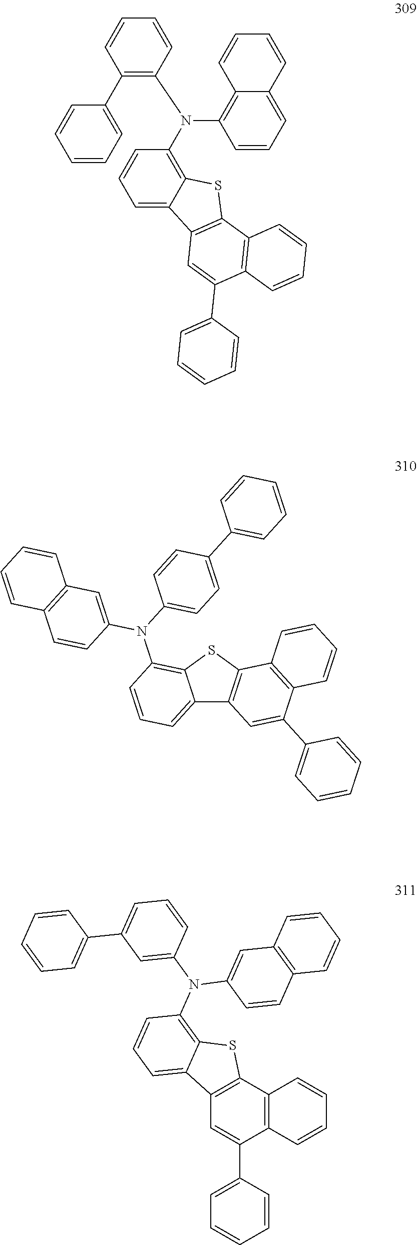 [SMILES: c1ccc(-c2ccccc2N(c2cccc3ccccc23)c2cccc3c2sc2c4ccccc4c(-c4ccccc4)cc32)cc1 ; c1ccc(-c2ccc(N(c3ccc4ccccc4c3)c3cccc4c3sc3c5ccccc5c(-c5ccccc5)cc43)cc2)cc1 ; c1ccc(-c2cccc(N(c3ccc4ccccc4c3)c3cccc4c3sc3c5ccccc5c(-c5ccccc5)cc43)c2)cc1]

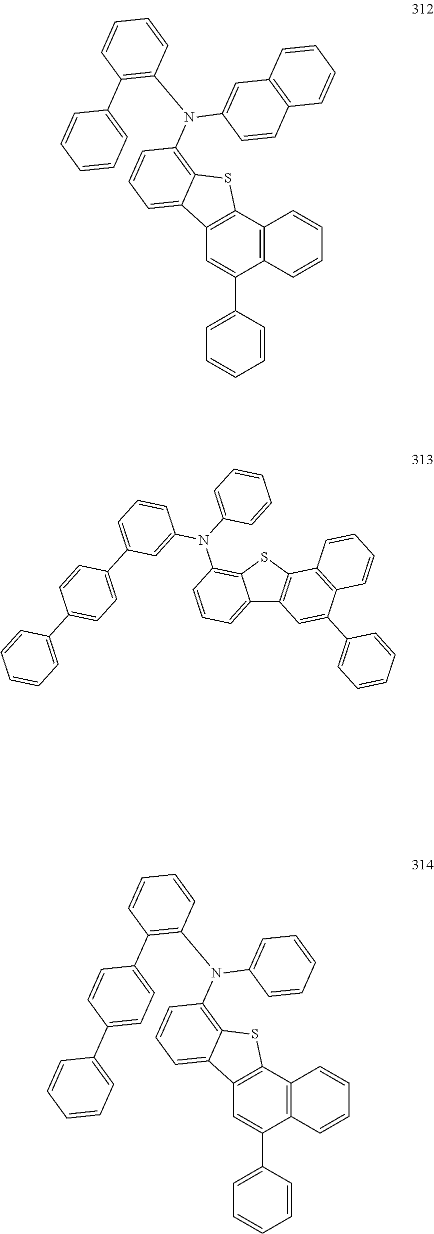 [SMILES: c1ccc(-c2ccccc2N(c2ccc3ccccc3c2)c2cccc3c2sc2c4ccccc4c(-c4ccccc4)cc32)cc1 ; c1ccc(-c2ccc(-c3cccc(N(c4ccccc4)c4cccc5c4sc4c6ccccc6c(-c6ccccc6)cc54)c3)cc2)cc1 ; c1ccc(-c2ccc(-c3ccccc3N(c3ccccc3)c3cccc4c3sc3c5ccccc5c(-c5ccccc5)cc43)cc2)cc1]

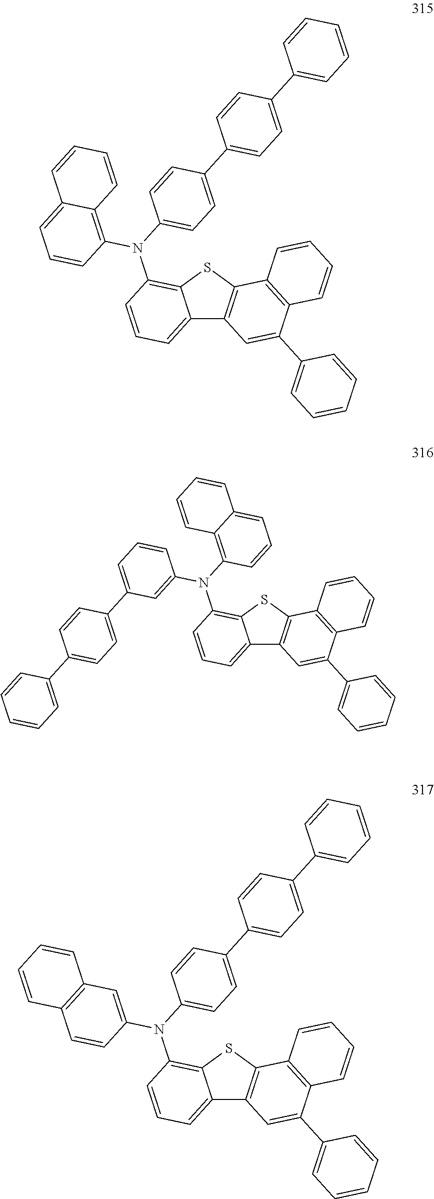 [SMILES: c1ccc(-c2ccc(-c3ccc(N(c4cccc5ccccc45)c4cccc5c4sc4c6ccccc6c(-c6ccccc6)cc54)cc3)cc2)cc1 ; c1ccc(-c2ccc(-c3cccc(N(c4cccc5ccccc45)c4cccc5c4sc4c6ccccc6c(-c6ccccc6)cc54)c3)cc2)cc1 ; c1ccc(-c2ccc(-c3ccc(N(c4ccc5ccccc5c4)c4cccc5c4sc4c6ccccc6c(-c6ccccc6)cc54)cc3)cc2)cc1]

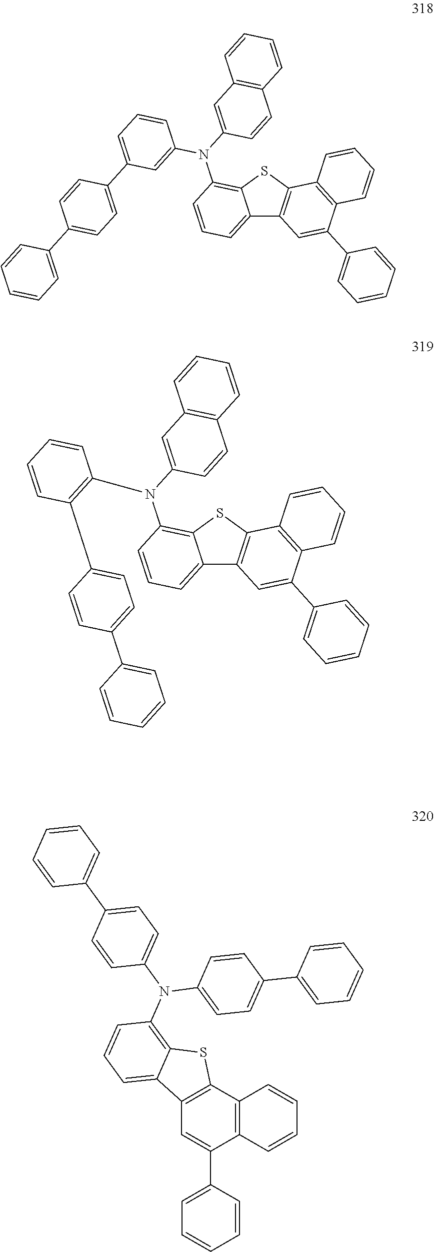 [SMILES: c1ccc(-c2ccc(-c3cccc(N(c4ccc5ccccc5c4)c4cccc5c4sc4c6ccccc6c(-c6ccccc6)cc54)c3)cc2)cc1 ; c1ccc(-c2ccc(-c3ccccc3N(c3ccc4ccccc4c3)c3cccc4c3sc3c5ccccc5c(-c5ccccc5)cc43)cc2)cc1 ; c1ccc(-c2ccc(N(c3ccc(-c4ccccc4)cc3)c3cccc4c3sc3c5ccccc5c(-c5ccccc5)cc43)cc2)cc1]

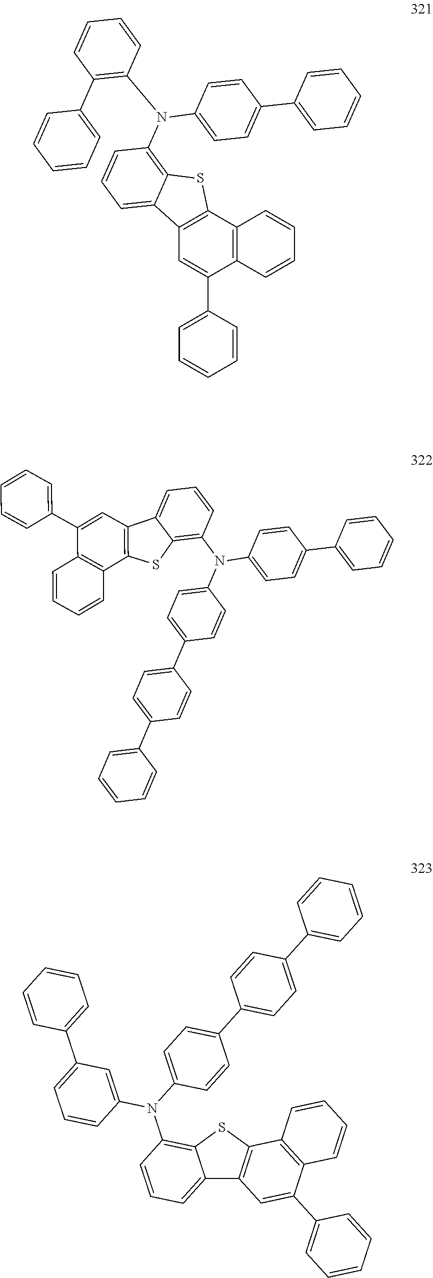 [SMILES: c1ccc(-c2ccc(N(c3ccccc3-c3ccccc3)c3cccc4c3sc3c5ccccc5c(-c5ccccc5)cc43)cc2)cc1 ; c1ccc(-c2ccc(-c3ccc(N(c4ccc(-c5ccccc5)cc4)c4cccc5c4sc4c6ccccc6c(-c6ccccc6)cc54)cc3)cc2)cc1 ; c1ccc(-c2ccc(-c3ccc(N(c4cccc(-c5ccccc5)c4)c4cccc5c4sc4c6ccccc6c(-c6ccccc6)cc54)cc3)cc2)cc1]

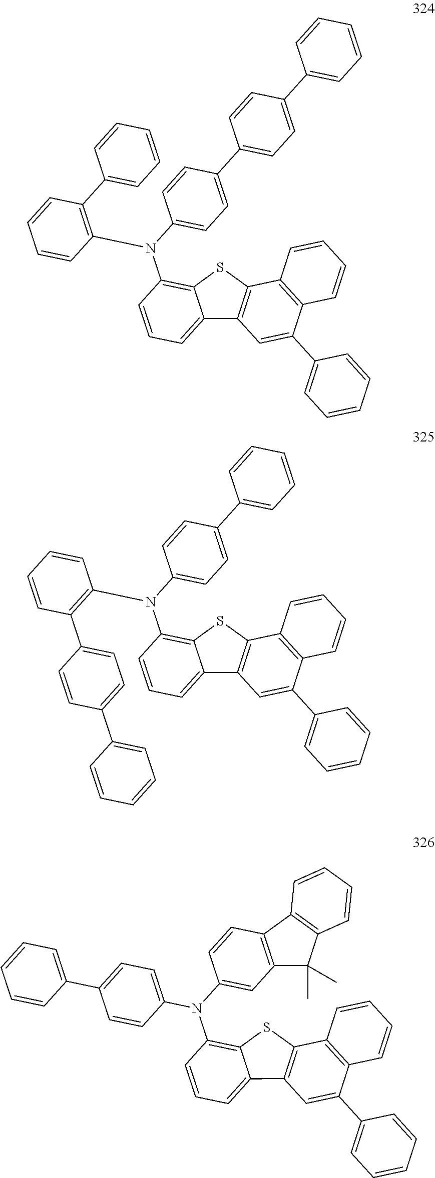 [SMILES: c1ccc(-c2ccc(-c3ccc(N(c4ccccc4-c4ccccc4)c4cccc5c4sc4c6ccccc6c(-c6ccccc6)cc54)cc3)cc2)cc1 ; c1ccc(-c2ccc(-c3ccccc3N(c3ccc(-c4ccccc4)cc3)c3cccc4c3sc3c5ccccc5c(-c5ccccc5)cc43)cc2)cc1 ; CC1(C)c2ccccc2-c2ccc(N(c3ccc(-c4ccccc4)cc3)c3cccc4c3sc3c5ccccc5c(-c5ccccc5)cc43)cc21]

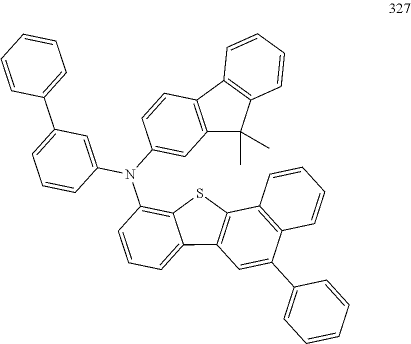 [SMILES: CC1(C)c2ccccc2-c2ccc(N(c3cccc(-c4ccccc4)c3)c3cccc4c3sc3c5ccccc5c(-c5ccccc5)cc43)cc21]

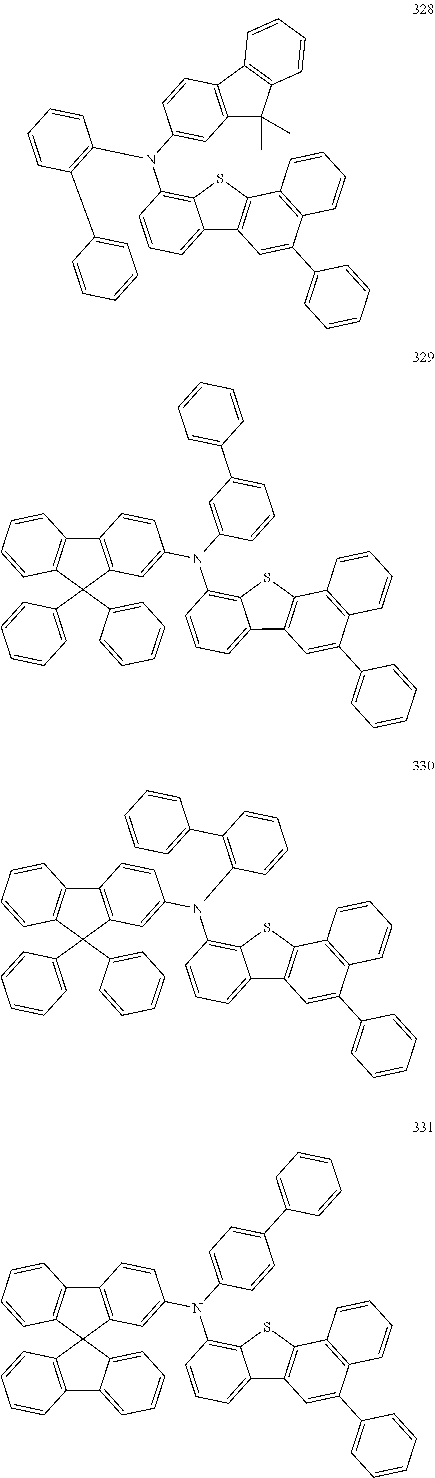 [SMILES: CC1(C)c2ccccc2-c2ccc(N(c3ccccc3-c3ccccc3)c3cccc4c3sc3c5ccccc5c(-c5ccccc5)cc43)cc21 ; c1ccc(-c2cccc(N(c3ccc4c(c3)C(c3ccccc3)(c3ccccc3)c3ccccc3-4)c3cccc4c3sc3c5ccccc5c(-c5ccccc5)cc43)c2)cc1 ; c1ccc(-c2ccccc2N(c2ccc3c(c2)C(c2ccccc2)(c2ccccc2)c2ccccc2-3)c2cccc3c2sc2c4ccccc4c(-c4ccccc4)cc32)cc1 ; c1ccc(-c2ccc(N(c3ccc4c(c3)C3(c5ccccc5-c5ccccc53)c3ccccc3-4)c3cccc4c3sc3c5ccccc5c(-c5ccccc5)cc43)cc2)cc1]

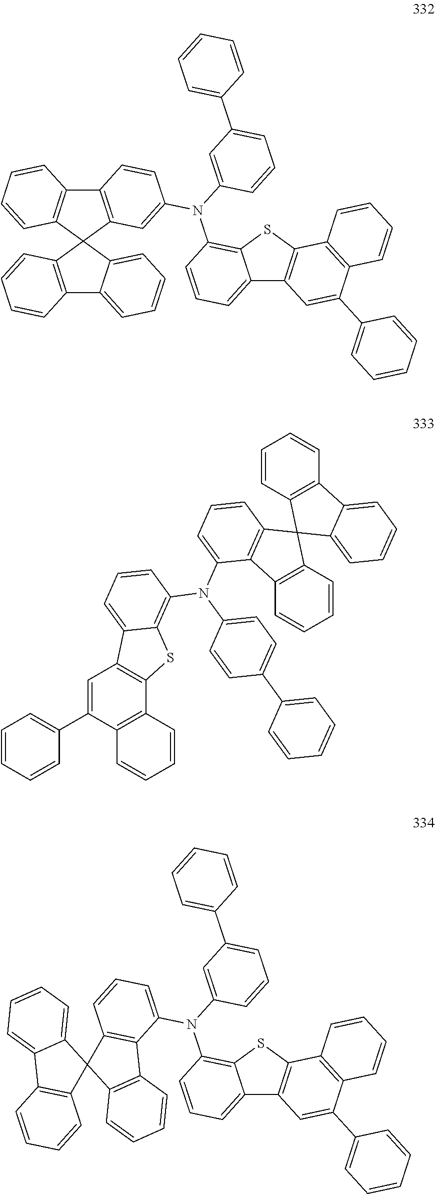 [SMILES: c1ccc(-c2cccc(N(c3ccc4c(c3)C3(c5ccccc5-c5ccccc53)c3ccccc3-4)c3cccc4c3sc3c5ccccc5c(-c5ccccc5)cc43)c2)cc1 ; c1ccc(-c2ccc(N(c3cccc4c3-c3ccccc3C43c4ccccc4-c4ccccc43)c3cccc4c3sc3c5ccccc5c(-c5ccccc5)cc43)cc2)cc1 ; c1ccc(-c2cccc(N(c3cccc4c3-c3ccccc3C43c4ccccc4-c4ccccc43)c3cccc4c3sc3c5ccccc5c(-c5ccccc5)cc43)c2)cc1]

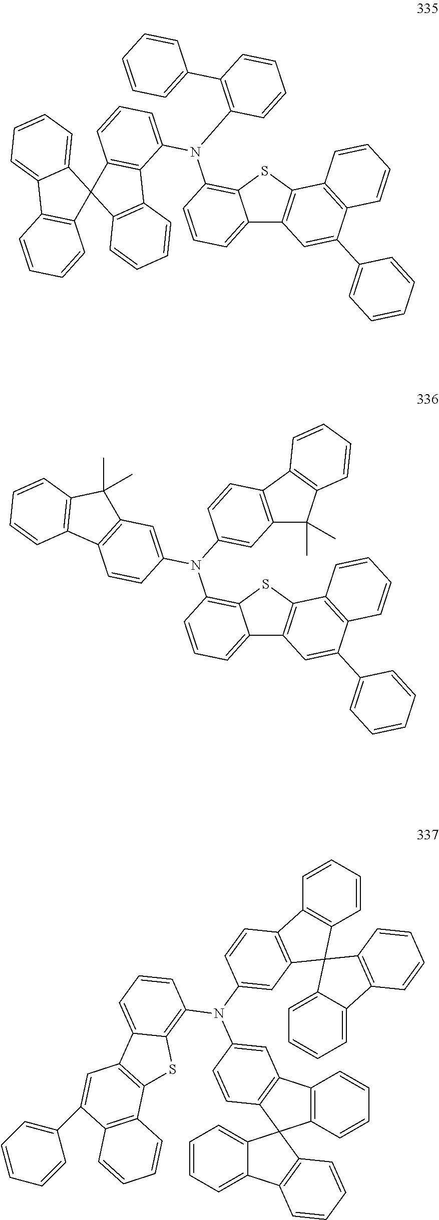 [SMILES: c1ccc(-c2ccccc2N(c2cccc3c2-c2ccccc2C32c3ccccc3-c3ccccc32)c2cccc3c2sc2c4ccccc4c(-c4ccccc4)cc32)cc1 ; CC1(C)c2ccccc2-c2ccc(N(c3ccc4c(c3)C(C)(C)c3ccccc3-4)c3cccc4c3sc3c5ccccc5c(-c5ccccc5)cc43)cc21 ; c1ccc(-c2cc3c4cccc(N(c5ccc6c(c5)-c5ccccc5C65c6ccccc6-c6ccccc65)c5ccc6c(c5)C5(c7ccccc7-c7ccccc75)c5ccccc5-6)c4sc3c3ccccc23)cc1]

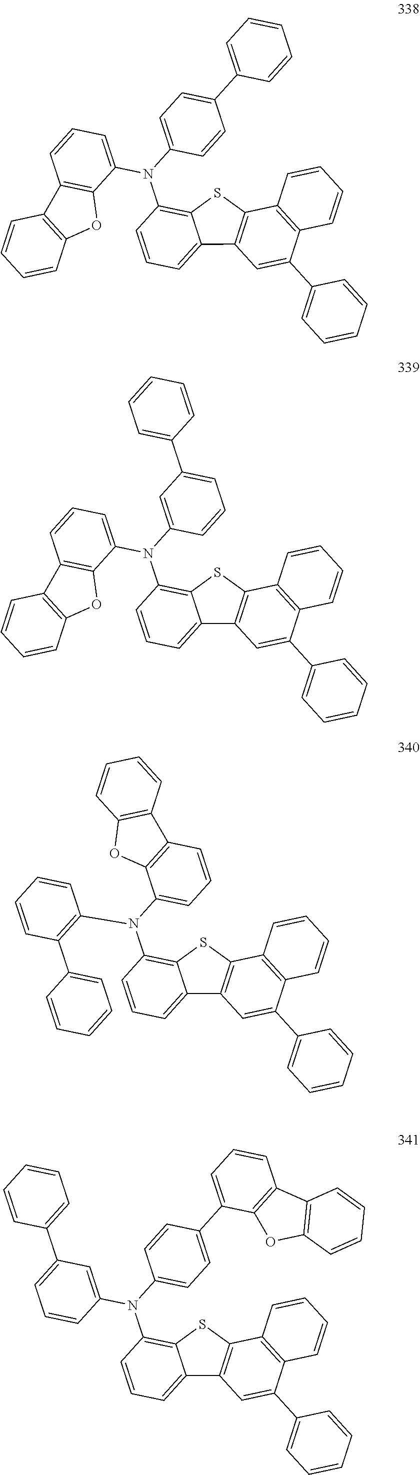 [SMILES: c1ccc(-c2ccc(N(c3cccc4c3oc3ccccc34)c3cccc4c3sc3c5ccccc5c(-c5ccccc5)cc43)cc2)cc1 ; c1ccc(-c2cccc(N(c3cccc4c3oc3ccccc34)c3cccc4c3sc3c5ccccc5c(-c5ccccc5)cc43)c2)cc1 ; c1ccc(-c2ccccc2N(c2cccc3c2oc2ccccc23)c2cccc3c2sc2c4ccccc4c(-c4ccccc4)cc32)cc1 ; c1ccc(-c2cccc(N(c3ccc(-c4cccc5c4oc4ccccc45)cc3)c3cccc4c3sc3c5ccccc5c(-c5ccccc5)cc43)c2)cc1]

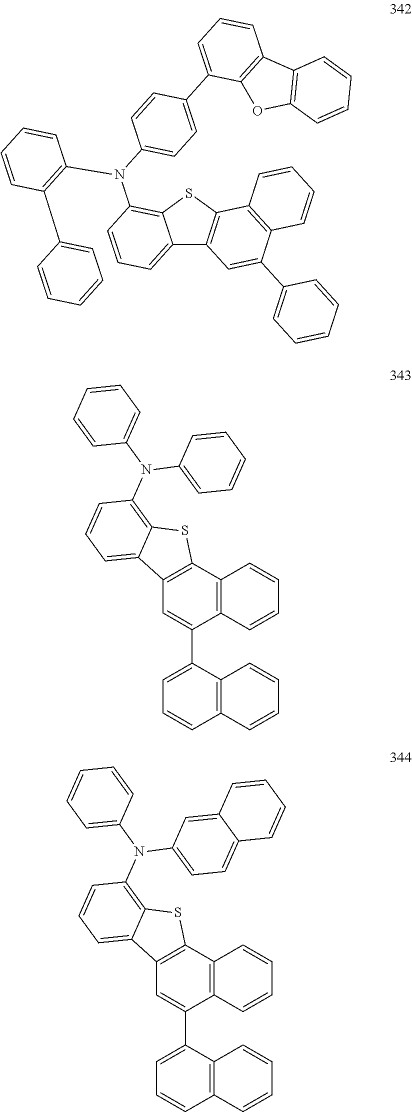 [SMILES: c1ccc(-c2ccccc2N(c2ccc(-c3cccc4c3oc3ccccc34)cc2)c2cccc3c2sc2c4ccccc4c(-c4ccccc4)cc32)cc1 ; c1ccc(N(c2ccccc2)c2cccc3c2sc2c4ccccc4c(-c4cccc5ccccc45)cc32)cc1 ; c1ccc(N(c2ccc3ccccc3c2)c2cccc3c2sc2c4ccccc4c(-c4cccc5ccccc45)cc32)cc1]

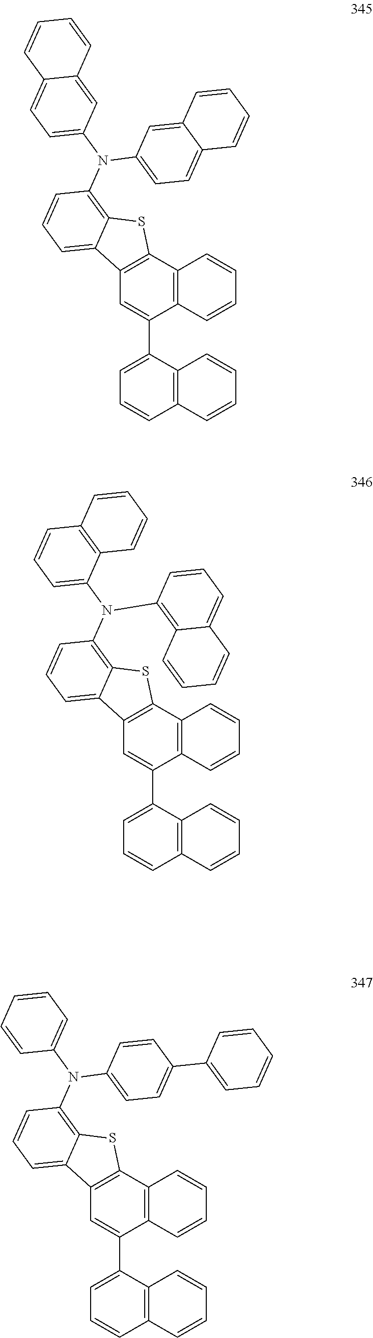 [SMILES: c1ccc2cc(N(c3ccc4ccccc4c3)c3cccc4c3sc3c5ccccc5c(-c5cccc6ccccc56)cc43)ccc2c1 ; c1ccc2c(-c3cc4c5cccc(N(c6cccc7ccccc67)c6cccc7ccccc67)c5sc4c4ccccc34)cccc2c1 ; c1ccc(-c2ccc(N(c3ccccc3)c3cccc4c3sc3c5ccccc5c(-c5cccc6ccccc56)cc43)cc2)cc1]

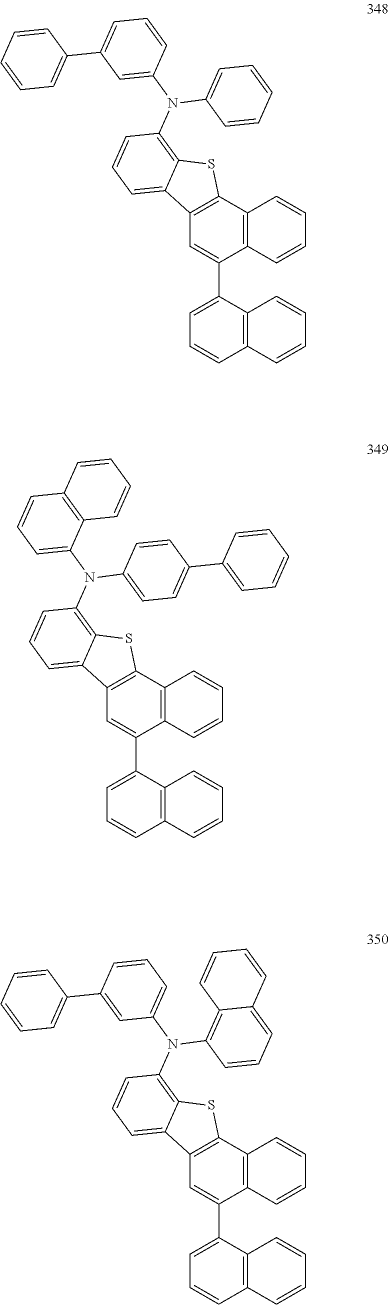 [SMILES: c1ccc(-c2cccc(N(c3ccccc3)c3cccc4c3sc3c5ccccc5c(-c5cccc6ccccc56)cc43)c2)cc1 ; c1ccc(-c2ccc(N(c3cccc4ccccc34)c3cccc4c3sc3c5ccccc5c(-c5cccc6ccccc56)cc43)cc2)cc1 ; c1ccc(-c2cccc(N(c3cccc4ccccc34)c3cccc4c3sc3c5ccccc5c(-c5cccc6ccccc56)cc43)c2)cc1]

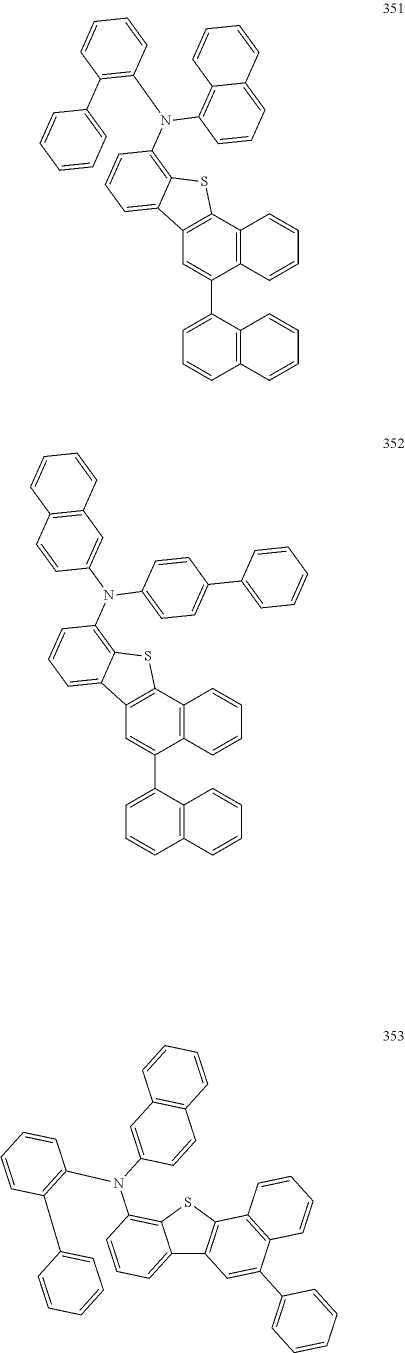 [SMILES: c1ccc(-c2ccccc2N(c2cccc3ccccc23)c2cccc3c2sc2c4ccccc4c(-c4cccc5ccccc45)cc32)cc1 ; c1ccc(-c2ccc(N(c3ccc4ccccc4c3)c3cccc4c3sc3c5ccccc5c(-c5cccc6ccccc56)cc43)cc2)cc1 ; c1ccc(-c2ccccc2N(c2ccc3ccccc3c2)c2cccc3c2sc2c4ccccc4c(-c4ccccc4)cc32)cc1]

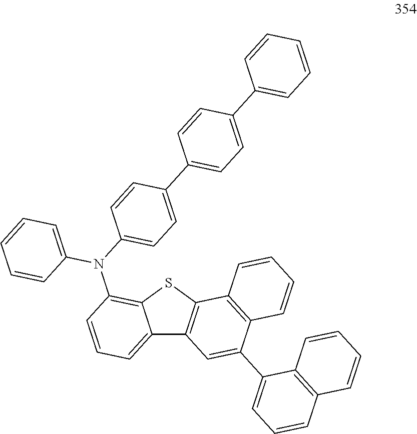 [SMILES: c1ccc(-c2ccc(-c3ccc(N(c4ccccc4)c4cccc5c4sc4c6ccccc6c(-c6cccc7ccccc67)cc54)cc3)cc2)cc1]

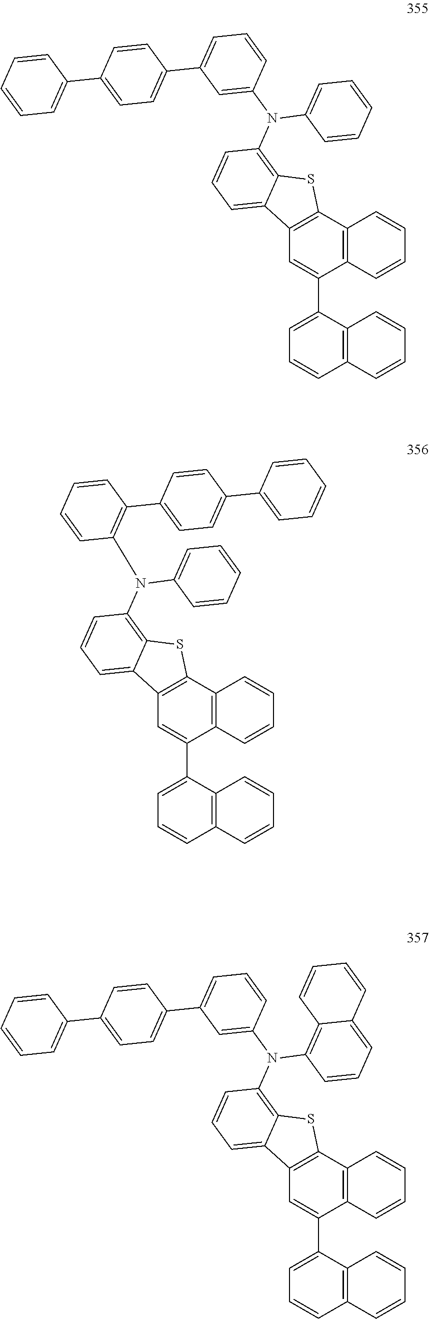 [SMILES: c1ccc(-c2ccc(-c3cccc(N(c4ccccc4)c4cccc5c4sc4c6ccccc6c(-c6cccc7ccccc67)cc54)c3)cc2)cc1 ; c1ccc(-c2ccc(-c3ccccc3N(c3ccccc3)c3cccc4c3sc3c5ccccc5c(-c5cccc6ccccc56)cc43)cc2)cc1 ; c1ccc(-c2ccc(-c3cccc(N(c4cccc5ccccc45)c4cccc5c4sc4c6ccccc6c(-c6cccc7ccccc67)cc54)c3)cc2)cc1]

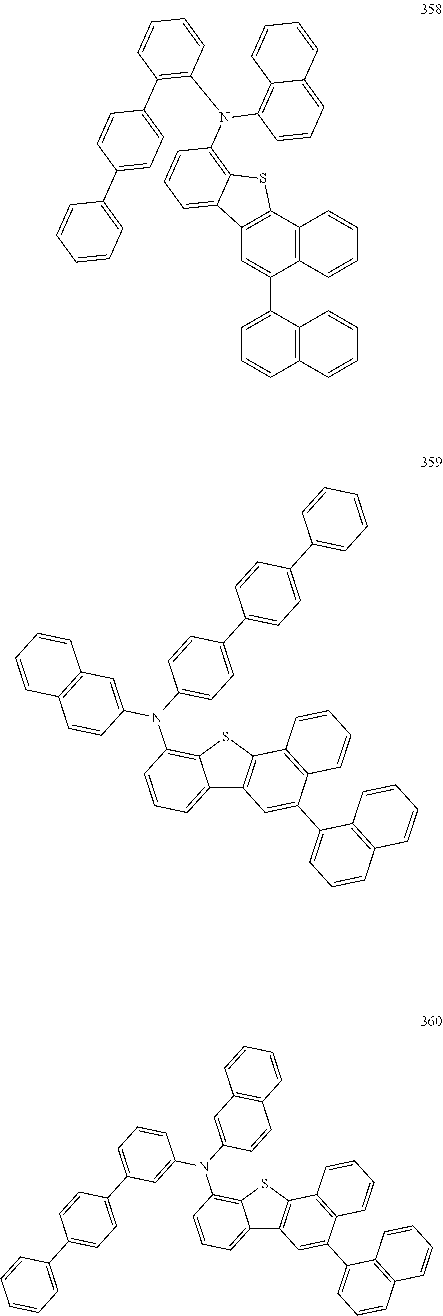 [SMILES: c1ccc(-c2ccc(-c3ccccc3N(c3cccc4ccccc34)c3cccc4c3sc3c5ccccc5c(-c5cccc6ccccc56)cc43)cc2)cc1 ; c1ccc(-c2ccc(-c3ccc(N(c4ccc5ccccc5c4)c4cccc5c4sc4c6ccccc6c(-c6cccc7ccccc67)cc54)cc3)cc2)cc1 ; c1ccc(-c2ccc(-c3cccc(N(c4ccc5ccccc5c4)c4cccc5c4sc4c6ccccc6c(-c6cccc7ccccc67)cc54)c3)cc2)cc1]

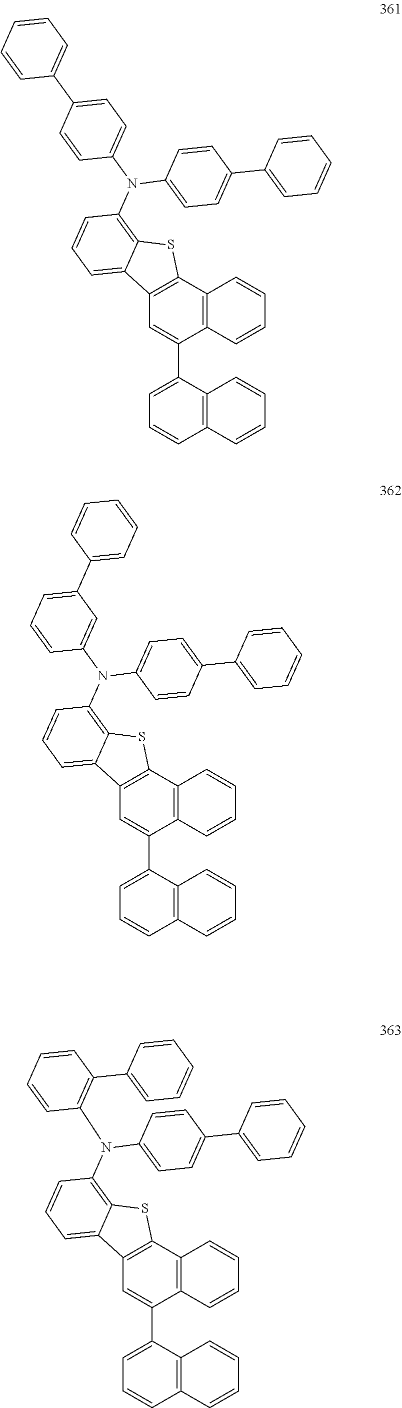 [SMILES: c1ccc(-c2ccc(N(c3ccc(-c4ccccc4)cc3)c3cccc4c3sc3c5ccccc5c(-c5cccc6ccccc56)cc43)cc2)cc1 ; c1ccc(-c2ccc(N(c3cccc(-c4ccccc4)c3)c3cccc4c3sc3c5ccccc5c(-c5cccc6ccccc56)cc43)cc2)cc1 ; c1ccc(-c2ccc(N(c3ccccc3-c3ccccc3)c3cccc4c3sc3c5ccccc5c(-c5cccc6ccccc56)cc43)cc2)cc1]

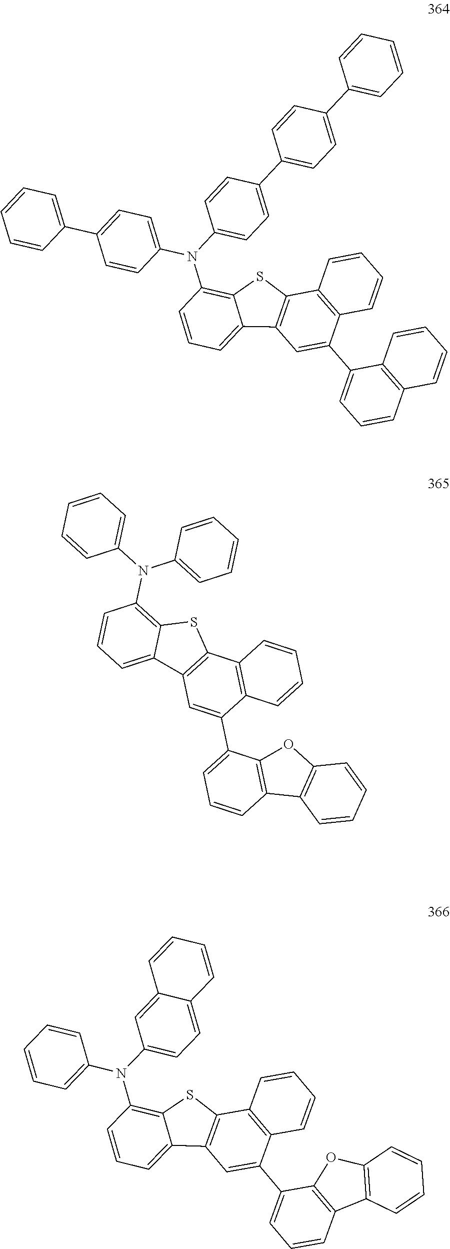 [SMILES: c1ccc(-c2ccc(-c3ccc(N(c4ccc(-c5ccccc5)cc4)c4cccc5c4sc4c6ccccc6c(-c6cccc7ccccc67)cc54)cc3)cc2)cc1 ; c1ccc(N(c2ccccc2)c2cccc3c2sc2c4ccccc4c(-c4cccc5c4oc4ccccc45)cc32)cc1 ; c1ccc(N(c2ccc3ccccc3c2)c2cccc3c2sc2c4ccccc4c(-c4cccc5c4oc4ccccc45)cc32)cc1]

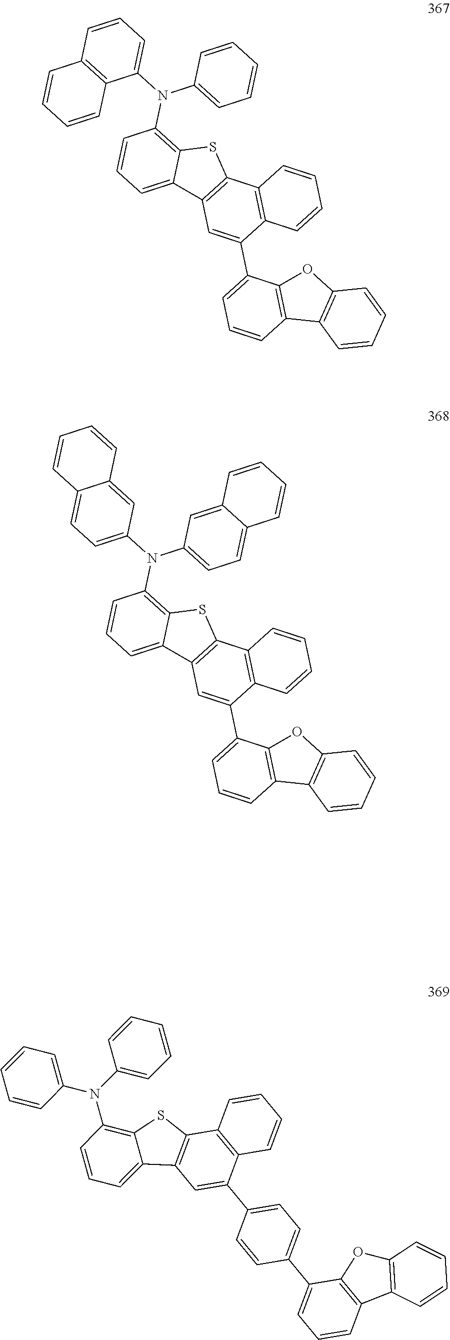 [SMILES: c1ccc(N(c2cccc3ccccc23)c2cccc3c2sc2c4ccccc4c(-c4cccc5c4oc4ccccc45)cc32)cc1 ; c1ccc2cc(N(c3ccc4ccccc4c3)c3cccc4c3sc3c5ccccc5c(-c5cccc6c5oc5ccccc56)cc43)ccc2c1 ; c1ccc(N(c2ccccc2)c2cccc3c2sc2c4ccccc4c(-c4ccc(-c5cccc6c5oc5ccccc56)cc4)cc32)cc1]

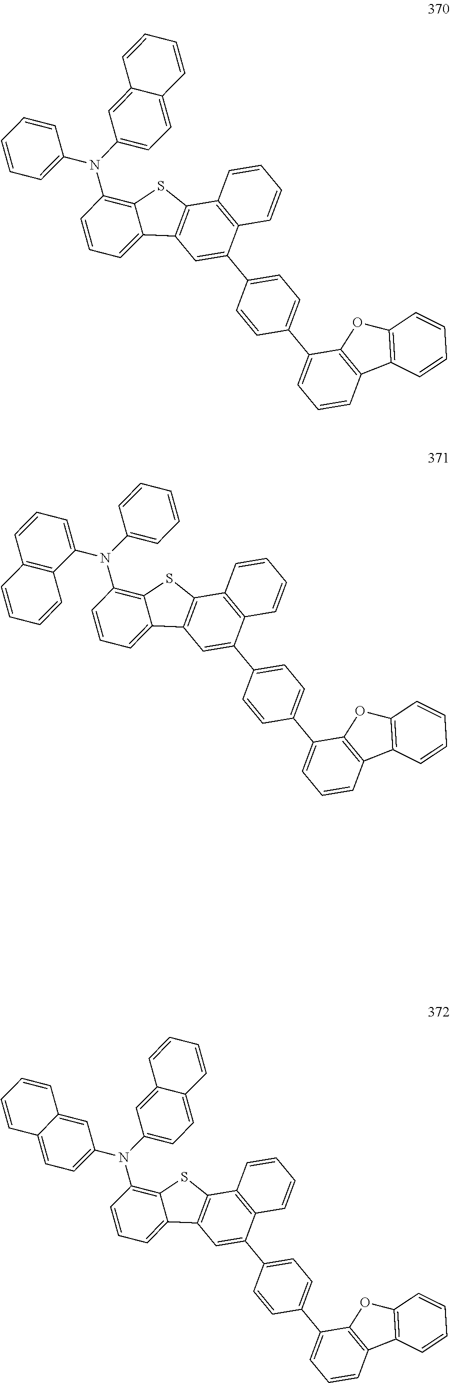 [SMILES: c1ccc(N(c2ccc3ccccc3c2)c2cccc3c2sc2c4ccccc4c(-c4ccc(-c5cccc6c5oc5ccccc56)cc4)cc32)cc1 ; c1ccc(N(c2cccc3ccccc23)c2cccc3c2sc2c4ccccc4c(-c4ccc(-c5cccc6c5oc5ccccc56)cc4)cc32)cc1 ; c1ccc2cc(N(c3ccc4ccccc4c3)c3cccc4c3sc3c5ccccc5c(-c5ccc(-c6cccc7c6oc6ccccc67)cc5)cc43)ccc2c1]

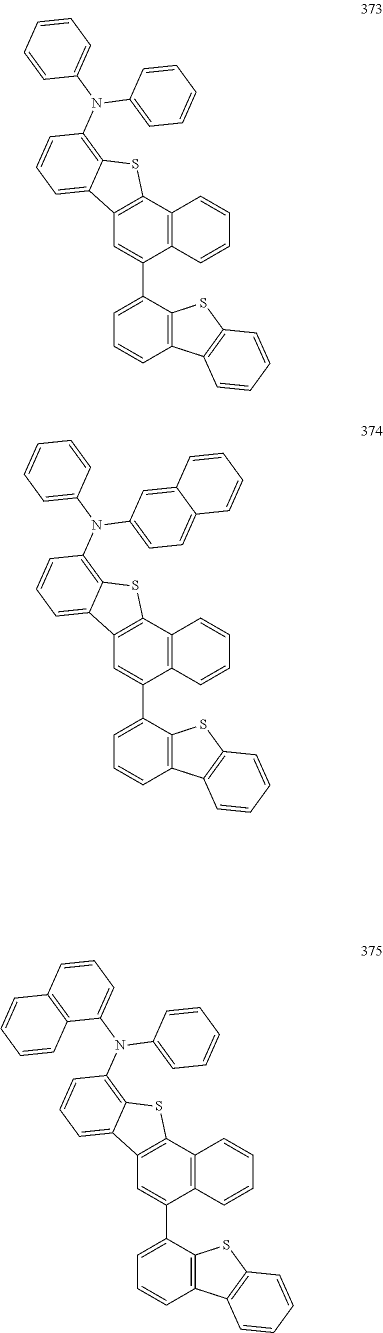 [SMILES: c1ccc(N(c2ccccc2)c2cccc3c2sc2c4ccccc4c(-c4cccc5c4sc4ccccc45)cc32)cc1 ; c1ccc(N(c2ccc3ccccc3c2)c2cccc3c2sc2c4ccccc4c(-c4cccc5c4sc4ccccc45)cc32)cc1 ; c1ccc(N(c2cccc3ccccc23)c2cccc3c2sc2c4ccccc4c(-c4cccc5c4sc4ccccc45)cc32)cc1]

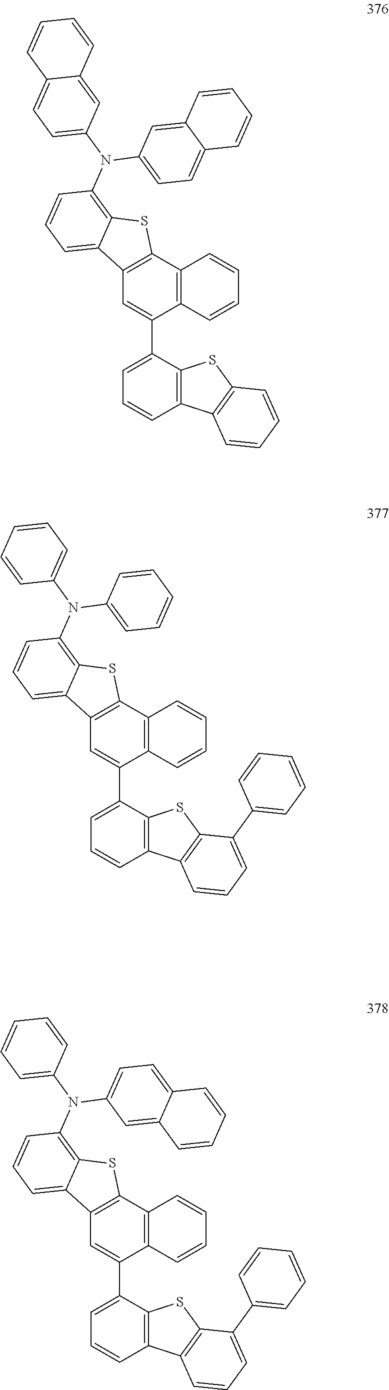 [SMILES: c1ccc2cc(N(c3ccc4ccccc4c3)c3cccc4c3sc3c5ccccc5c(-c5cccc6c5sc5ccccc56)cc43)ccc2c1 ; c1ccc(-c2cccc3c2sc2c(-c4cc5c6cccc(N(c7ccccc7)c7ccccc7)c6sc5c5ccccc45)cccc23)cc1 ; c1ccc(-c2cccc3c2sc2c(-c4cc5c6cccc(N(c7ccccc7)c7ccc8ccccc8c7)c6sc5c5ccccc45)cccc23)cc1]

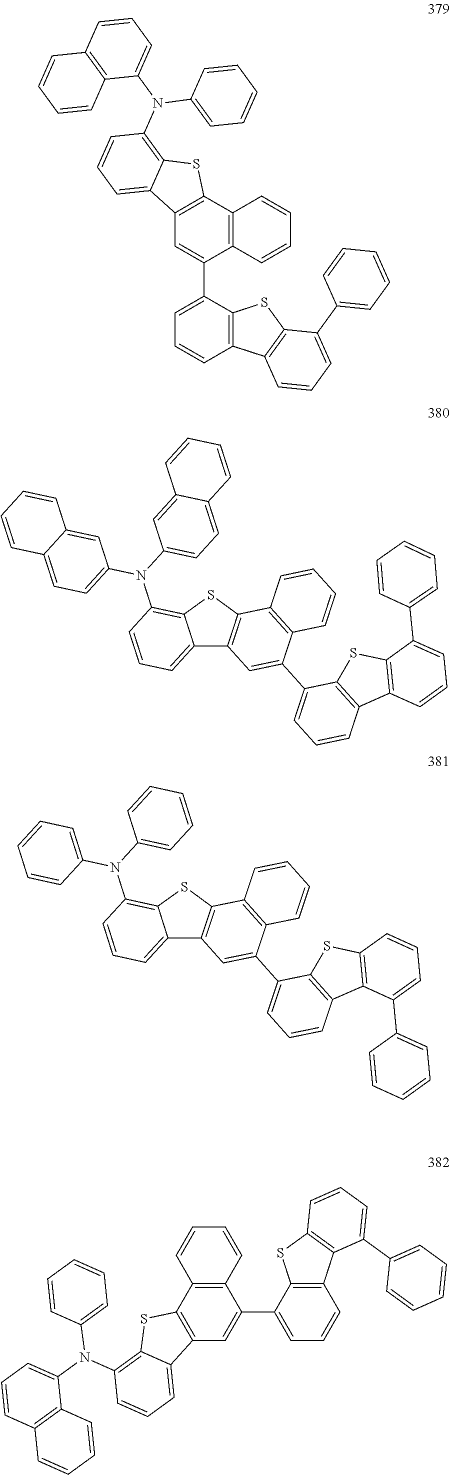 [SMILES: c1ccc(-c2cccc3c2sc2c(-c4cc5c6cccc(N(c7ccccc7)c7cccc8ccccc78)c6sc5c5ccccc45)cccc23)cc1 ; c1ccc(-c2cccc3c2sc2c(-c4cc5c6cccc(N(c7ccc8ccccc8c7)c7ccc8ccccc8c7)c6sc5c5ccccc45)cccc23)cc1 ; c1ccc(-c2cccc3sc4c(-c5cc6c7cccc(N(c8ccccc8)c8ccccc8)c7sc6c6ccccc56)cccc4c23)cc1 ; c1ccc(-c2cccc3sc4c(-c5cc6c7cccc(N(c8ccccc8)c8cccc9ccccc89)c7sc6c6ccccc56)cccc4c23)cc1]

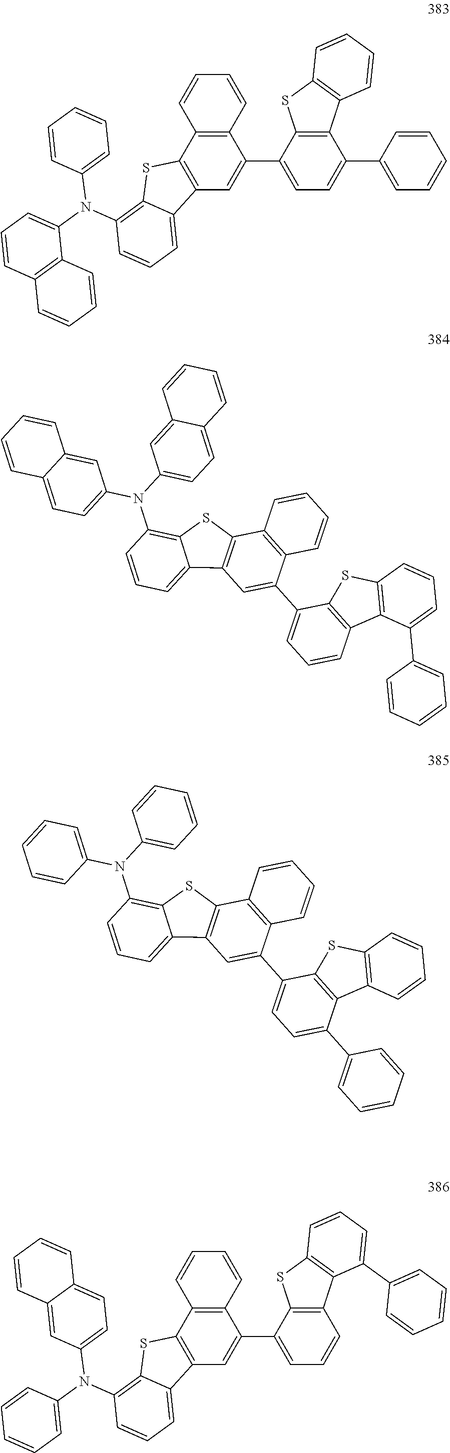 [SMILES: c1ccc(-c2ccc(-c3cc4c5cccc(N(c6ccccc6)c6cccc7ccccc67)c5sc4c4ccccc34)c3sc4ccccc4c23)cc1 ; c1ccc(-c2cccc3sc4c(-c5cc6c7cccc(N(c8ccc9ccccc9c8)c8ccc9ccccc9c8)c7sc6c6ccccc56)cccc4c23)cc1 ; c1ccc(-c2ccc(-c3cc4c5cccc(N(c6ccccc6)c6ccccc6)c5sc4c4ccccc34)c3sc4ccccc4c23)cc1 ; c1ccc(-c2cccc3sc4c(-c5cc6c7cccc(N(c8ccccc8)c8ccc9ccccc9c8)c7sc6c6ccccc56)cccc4c23)cc1]

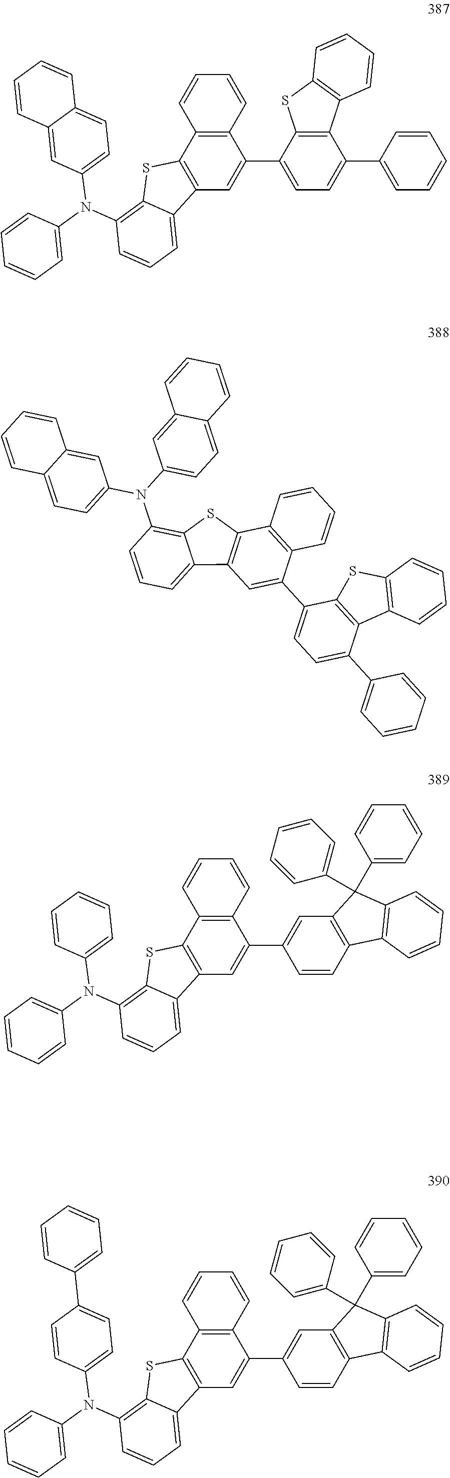 [SMILES: c1ccc(-c2ccc(-c3cc4c5cccc(N(c6ccccc6)c6ccc7ccccc7c6)c5sc4c4ccccc34)c3sc4ccccc4c23)cc1 ; c1ccc(-c2ccc(-c3cc4c5cccc(N(c6ccc7ccccc7c6)c6ccc7ccccc7c6)c5sc4c4ccccc34)c3sc4ccccc4c23)cc1 ; c1ccc(N(c2ccccc2)c2cccc3c2sc2c4ccccc4c(-c4ccc5c(c4)C(c4ccccc4)(c4ccccc4)c4ccccc4-5)cc32)cc1 ; c1ccc(-c2ccc(N(c3ccccc3)c3cccc4c3sc3c5ccccc5c(-c5ccc6c(c5)C(c5ccccc5)(c5ccccc5)c5ccccc5-6)cc43)cc2)cc1]

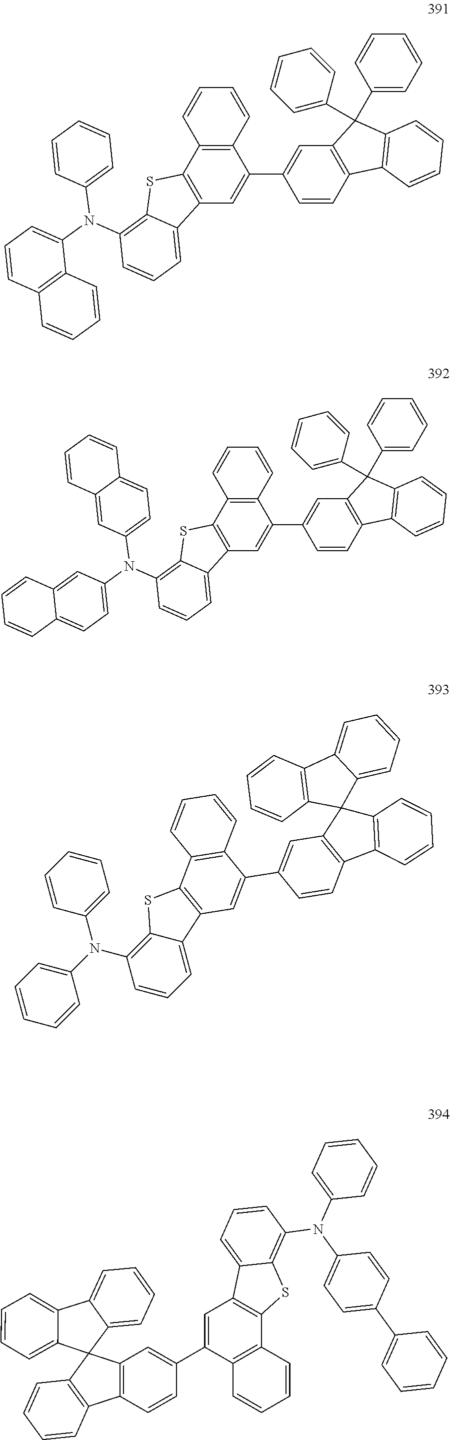 [SMILES: c1ccc(N(c2cccc3ccccc23)c2cccc3c2sc2c4ccccc4c(-c4ccc5c(c4)C(c4ccccc4)(c4ccccc4)c4ccccc4-5)cc32)cc1 ; c1ccc(C2(c3ccccc3)c3ccccc3-c3ccc(-c4cc5c6cccc(N(c7ccc8ccccc8c7)c7ccc8ccccc8c7)c6sc5c5ccccc45)cc32)cc1 ; c1ccc(N(c2ccccc2)c2cccc3c2sc2c4ccccc4c(-c4ccc5c(c4)C4(c6ccccc6-c6ccccc64)c4ccccc4-5)cc32)cc1 ; c1ccc(-c2ccc(N(c3ccccc3)c3cccc4c3sc3c5ccccc5c(-c5ccc6c(c5)C5(c7ccccc7-c7ccccc75)c5ccccc5-6)cc43)cc2)cc1]

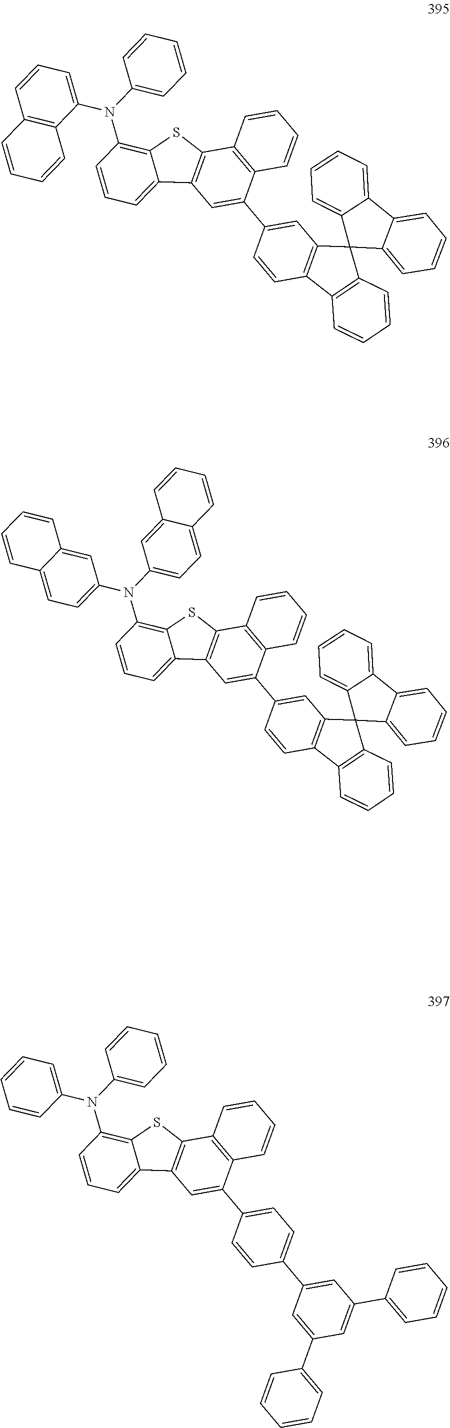 [SMILES: c1ccc(N(c2cccc3ccccc23)c2cccc3c2sc2c4ccccc4c(-c4ccc5c(c4)C4(c6ccccc6-c6ccccc64)c4ccccc4-5)cc32)cc1 ; c1ccc2c(c1)-c1ccccc1C21c2ccccc2-c2ccc(-c3cc4c5cccc(N(c6ccc7ccccc7c6)c6ccc7ccccc7c6)c5sc4c4ccccc34)cc21 ; c1ccc(-c2cc(-c3ccccc3)cc(-c3ccc(-c4cc5c6cccc(N(c7ccccc7)c7ccccc7)c6sc5c5ccccc45)cc3)c2)cc1]

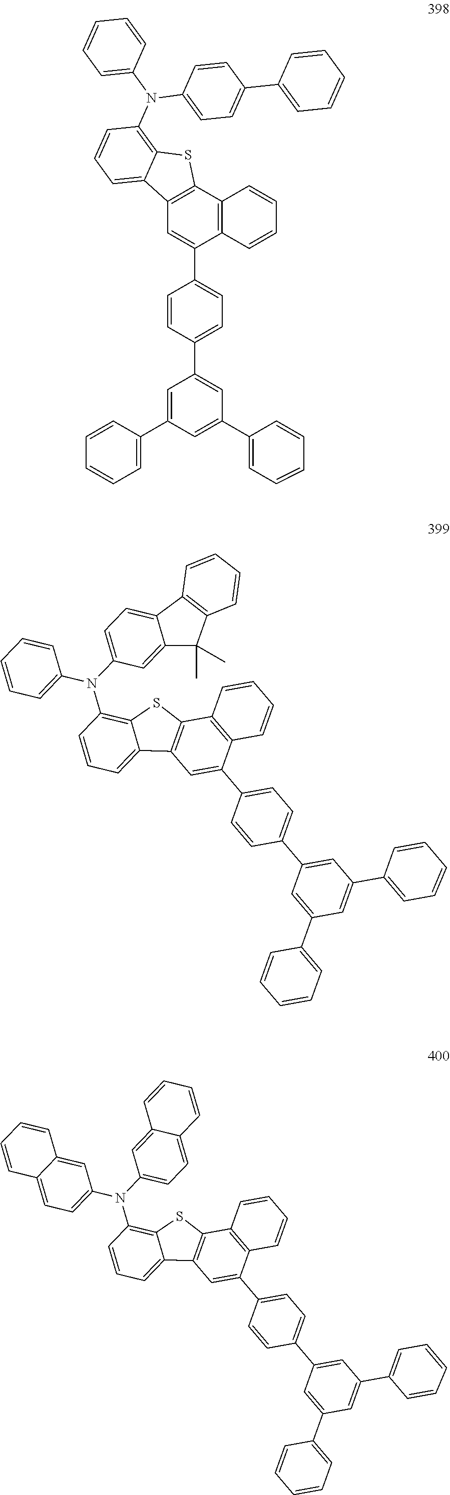 [SMILES: c1ccc(-c2ccc(N(c3ccccc3)c3cccc4c3sc3c5ccccc5c(-c5ccc(-c6cc(-c7ccccc7)cc(-c7ccccc7)c6)cc5)cc43)cc2)cc1 ; CC1(C)c2ccccc2-c2ccc(N(c3ccccc3)c3cccc4c3sc3c5ccccc5c(-c5ccc(-c6cc(-c7ccccc7)cc(-c7ccccc7)c6)cc5)cc43)cc21 ; c1ccc(-c2cc(-c3ccccc3)cc(-c3ccc(-c4cc5c6cccc(N(c7ccc8ccccc8c7)c7ccc8ccccc8c7)c6sc5c5ccccc45)cc3)c2)cc1]

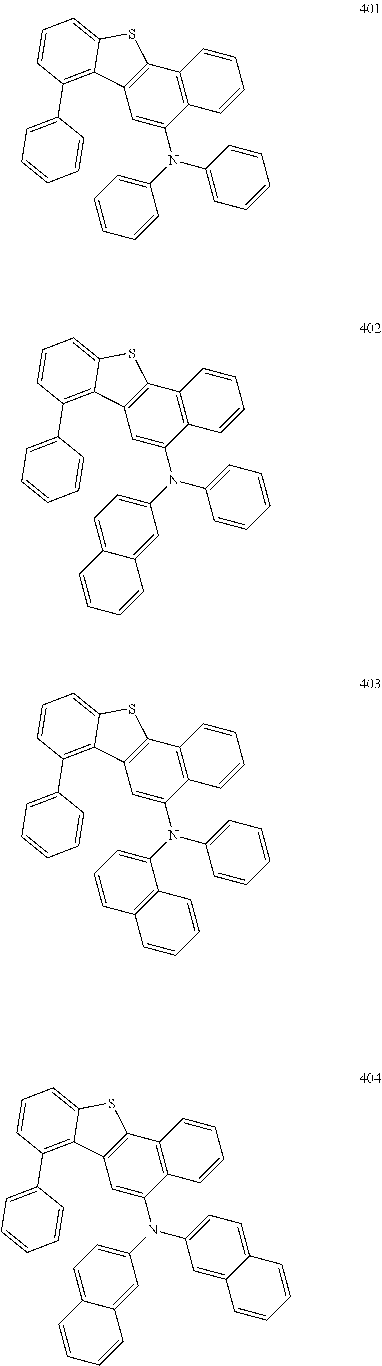 [SMILES: c1ccc(-c2cccc3sc4c5ccccc5c(N(c5ccccc5)c5ccccc5)cc4c23)cc1 ; c1ccc(-c2cccc3sc4c5ccccc5c(N(c5ccccc5)c5ccc6ccccc6c5)cc4c23)cc1 ; c1ccc(-c2cccc3sc4c5ccccc5c(N(c5ccccc5)c5cccc6ccccc56)cc4c23)cc1 ; c1ccc(-c2cccc3sc4c5ccccc5c(N(c5ccc6ccccc6c5)c5ccc6ccccc6c5)cc4c23)cc1]

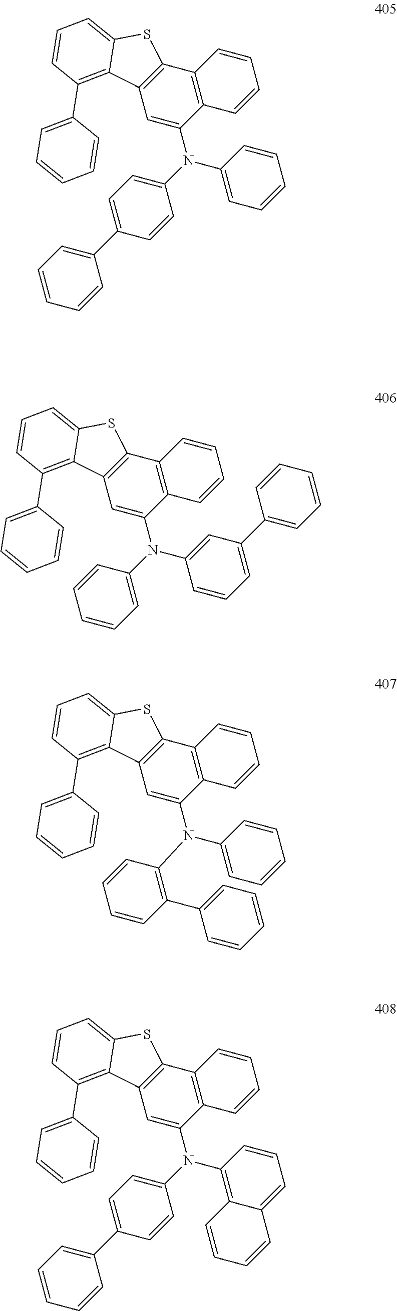 [SMILES: c1ccc(-c2ccc(N(c3ccccc3)c3cc4c(sc5cccc(-c6ccccc6)c54)c4ccccc34)cc2)cc1 ; c1ccc(-c2cccc(N(c3ccccc3)c3cc4c(sc5cccc(-c6ccccc6)c54)c4ccccc34)c2)cc1 ; c1ccc(-c2ccccc2N(c2ccccc2)c2cc3c(sc4cccc(-c5ccccc5)c43)c3ccccc23)cc1 ; c1ccc(-c2ccc(N(c3cccc4ccccc34)c3cc4c(sc5cccc(-c6ccccc6)c54)c4ccccc34)cc2)cc1]

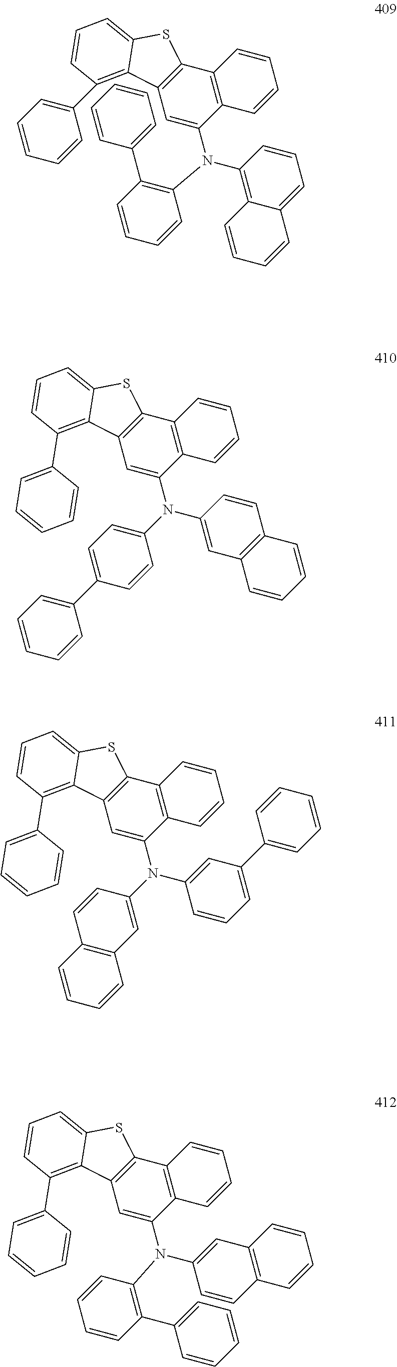 [SMILES: c1ccc(-c2ccccc2N(c2cccc3ccccc23)c2cc3c(sc4cccc(-c5ccccc5)c43)c3ccccc23)cc1 ; c1ccc(-c2ccc(N(c3ccc4ccccc4c3)c3cc4c(sc5cccc(-c6ccccc6)c54)c4ccccc34)cc2)cc1 ; c1ccc(-c2cccc(N(c3ccc4ccccc4c3)c3cc4c(sc5cccc(-c6ccccc6)c54)c4ccccc34)c2)cc1 ; c1ccc(-c2ccccc2N(c2ccc3ccccc3c2)c2cc3c(sc4cccc(-c5ccccc5)c43)c3ccccc23)cc1]

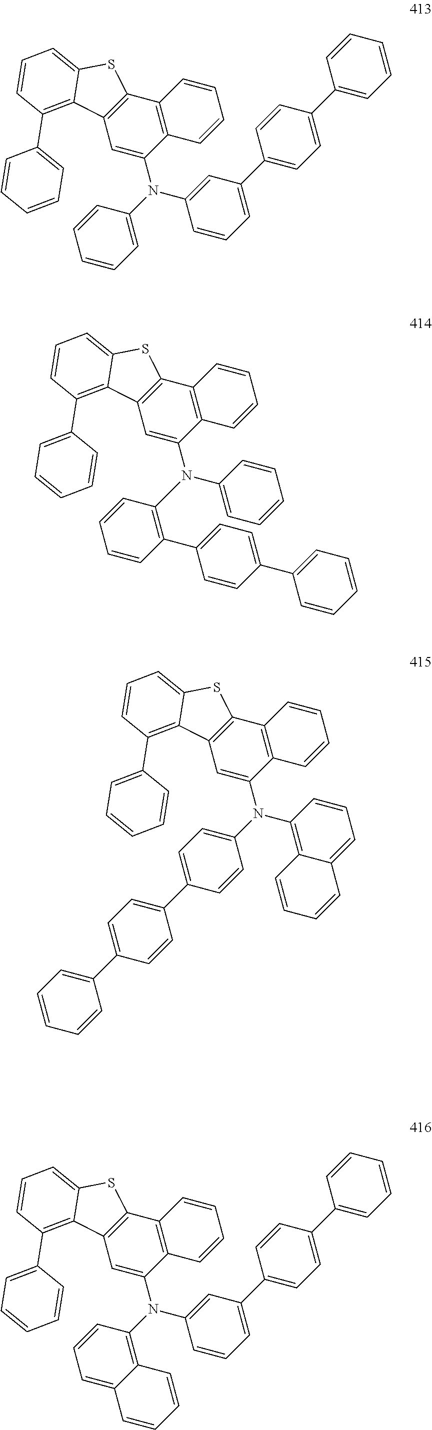 [SMILES: c1ccc(-c2ccc(-c3cccc(N(c4ccccc4)c4cc5c(sc6cccc(-c7ccccc7)c65)c5ccccc45)c3)cc2)cc1 ; c1ccc(-c2ccc(-c3ccccc3N(c3ccccc3)c3cc4c(sc5cccc(-c6ccccc6)c54)c4ccccc34)cc2)cc1 ; c1ccc(-c2ccc(-c3ccc(N(c4cccc5ccccc45)c4cc5c(sc6cccc(-c7ccccc7)c65)c5ccccc45)cc3)cc2)cc1 ; c1ccc(-c2ccc(-c3cccc(N(c4cccc5ccccc45)c4cc5c(sc6cccc(-c7ccccc7)c65)c5ccccc45)c3)cc2)cc1]

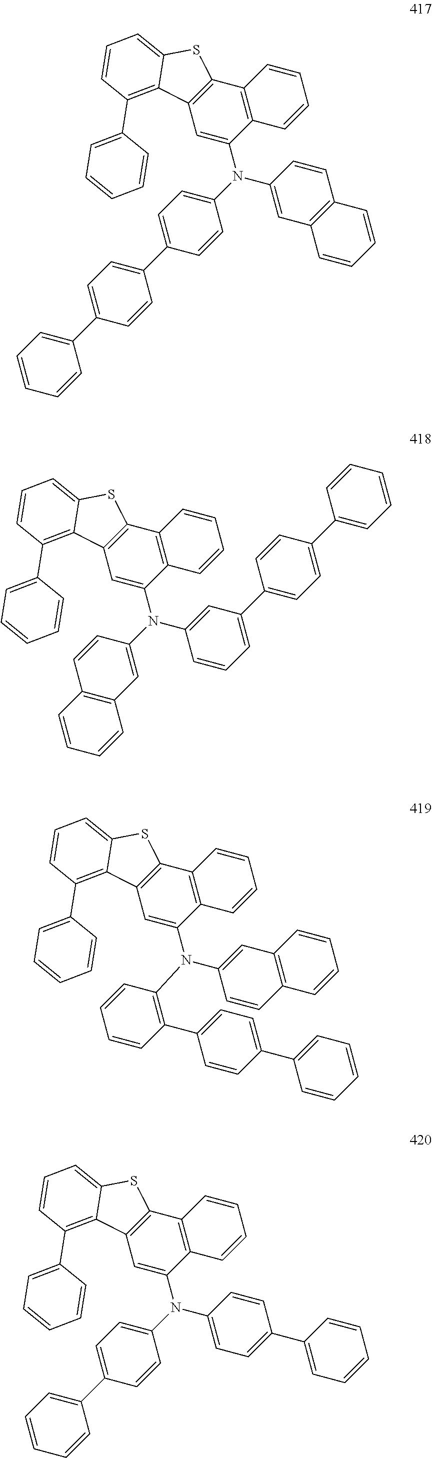 [SMILES: c1ccc(-c2ccc(-c3ccc(N(c4ccc5ccccc5c4)c4cc5c(sc6cccc(-c7ccccc7)c65)c5ccccc45)cc3)cc2)cc1 ; c1ccc(-c2ccc(-c3cccc(N(c4ccc5ccccc5c4)c4cc5c(sc6cccc(-c7ccccc7)c65)c5ccccc45)c3)cc2)cc1 ; c1ccc(-c2ccc(-c3ccccc3N(c3ccc4ccccc4c3)c3cc4c(sc5cccc(-c6ccccc6)c54)c4ccccc34)cc2)cc1 ; c1ccc(-c2ccc(N(c3ccc(-c4ccccc4)cc3)c3cc4c(sc5cccc(-c6ccccc6)c54)c4ccccc34)cc2)cc1]

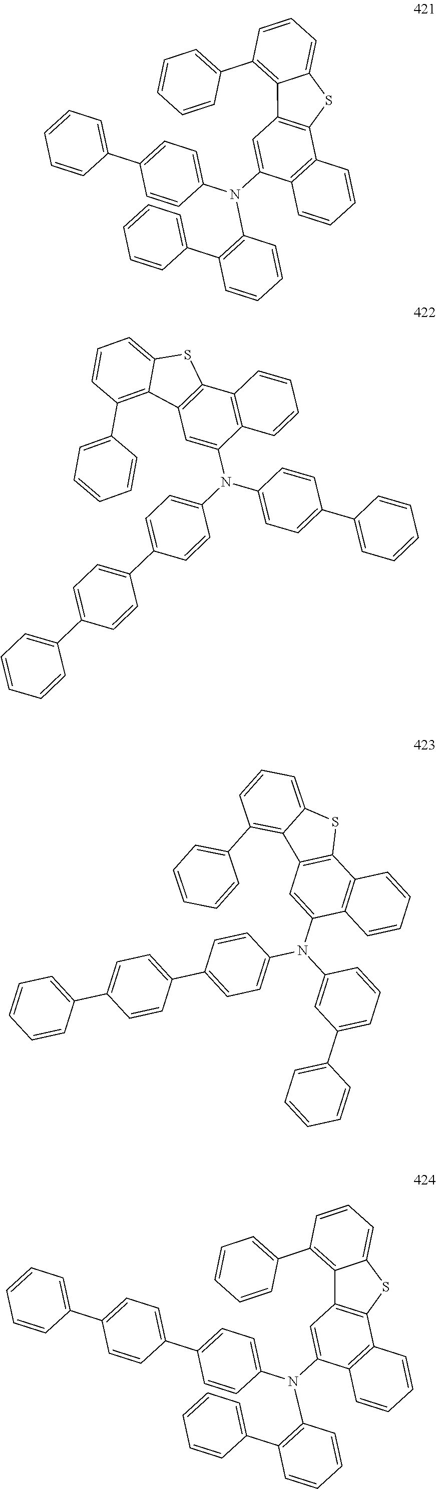 [SMILES: c1ccc(-c2ccc(N(c3ccccc3-c3ccccc3)c3cc4c(sc5cccc(-c6ccccc6)c54)c4ccccc34)cc2)cc1 ; c1ccc(-c2ccc(-c3ccc(N(c4ccc(-c5ccccc5)cc4)c4cc5c(sc6cccc(-c7ccccc7)c65)c5ccccc45)cc3)cc2)cc1 ; c1ccc(-c2ccc(-c3ccc(N(c4cccc(-c5ccccc5)c4)c4cc5c(sc6cccc(-c7ccccc7)c65)c5ccccc45)cc3)cc2)cc1 ; c1ccc(-c2ccc(-c3ccc(N(c4ccccc4-c4ccccc4)c4cc5c(sc6cccc(-c7ccccc7)c65)c5ccccc45)cc3)cc2)cc1]

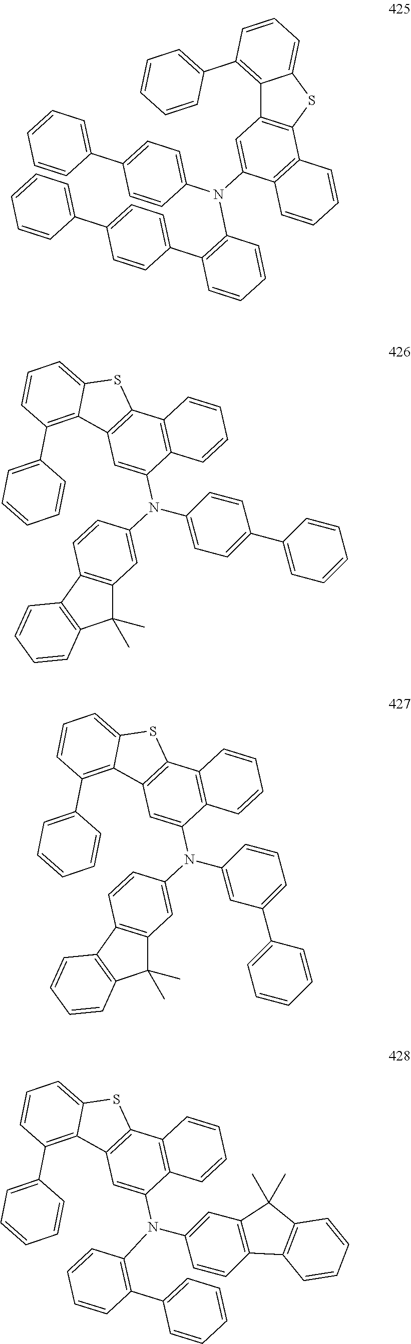 [SMILES: c1ccc(-c2ccc(-c3ccccc3N(c3ccc(-c4ccccc4)cc3)c3cc4c(sc5cccc(-c6ccccc6)c54)c4ccccc34)cc2)cc1 ; CC1(C)c2ccccc2-c2ccc(N(c3ccc(-c4ccccc4)cc3)c3cc4c(sc5cccc(-c6ccccc6)c54)c4ccccc34)cc21 ; CC1(C)c2ccccc2-c2ccc(N(c3cccc(-c4ccccc4)c3)c3cc4c(sc5cccc(-c6ccccc6)c54)c4ccccc34)cc21 ; CC1(C)c2ccccc2-c2ccc(N(c3ccccc3-c3ccccc3)c3cc4c(sc5cccc(-c6ccccc6)c54)c4ccccc34)cc21]

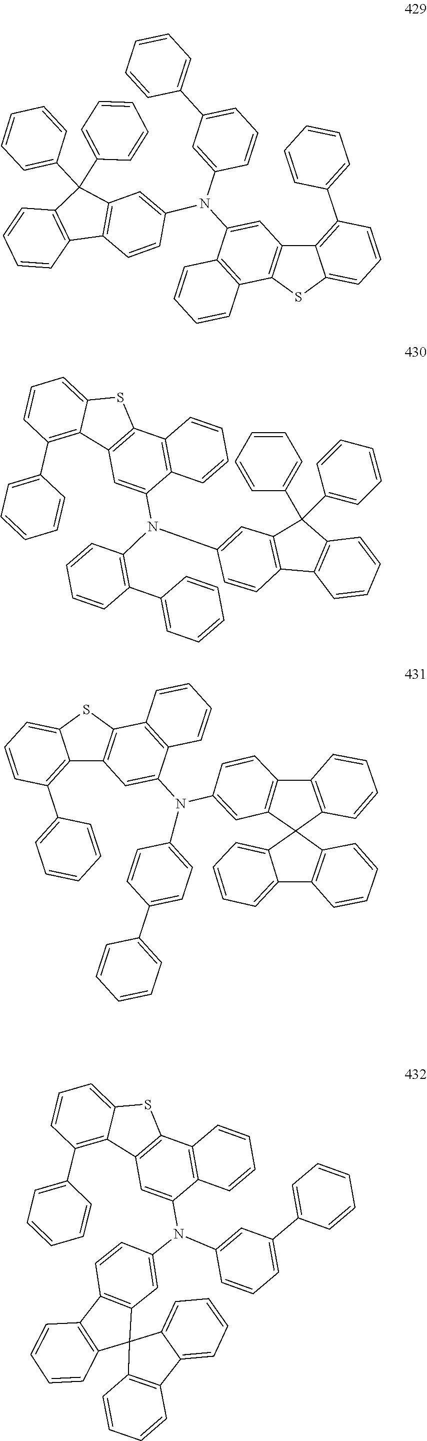

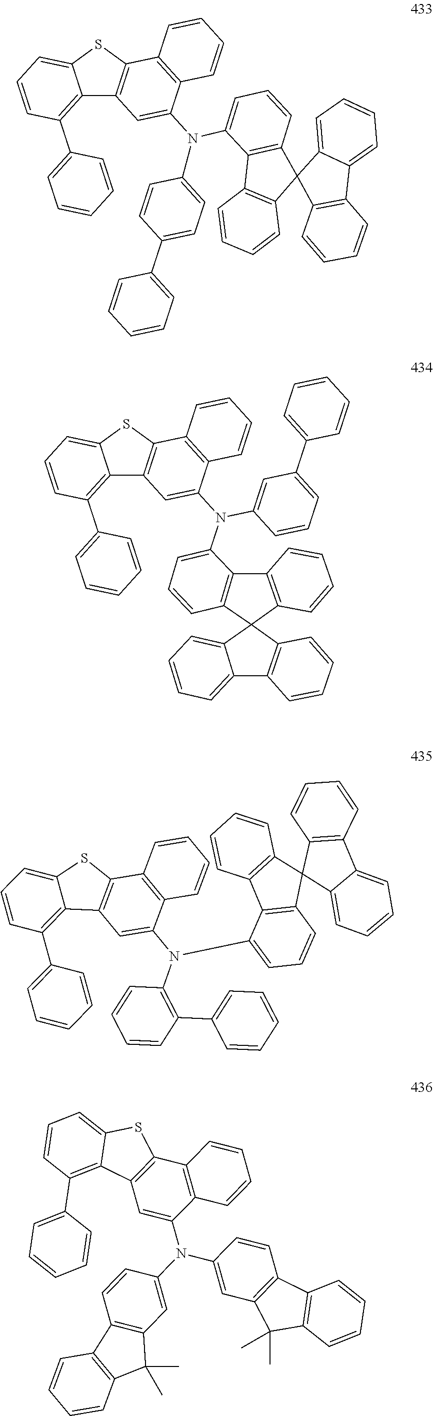 [SMILES: c1ccc(-c2ccc(N(c3cccc4c3-c3ccccc3C43c4ccccc4-c4ccccc43)c3cc4c(sc5cccc(-c6ccccc6)c54)c4ccccc34)cc2)cc1 ; c1ccc(-c2cccc(N(c3cccc4c3-c3ccccc3C43c4ccccc4-c4ccccc43)c3cc4c(sc5cccc(-c6ccccc6)c54)c4ccccc34)c2)cc1 ; c1ccc(-c2ccccc2N(c2cccc3c2-c2ccccc2C32c3ccccc3-c3ccccc32)c2cc3c(sc4cccc(-c5ccccc5)c43)c3ccccc23)cc1 ; CC1(C)c2ccccc2-c2ccc(N(c3ccc4c(c3)C(C)(C)c3ccccc3-4)c3cc4c(sc5cccc(-c6ccccc6)c54)c4ccccc34)cc21]

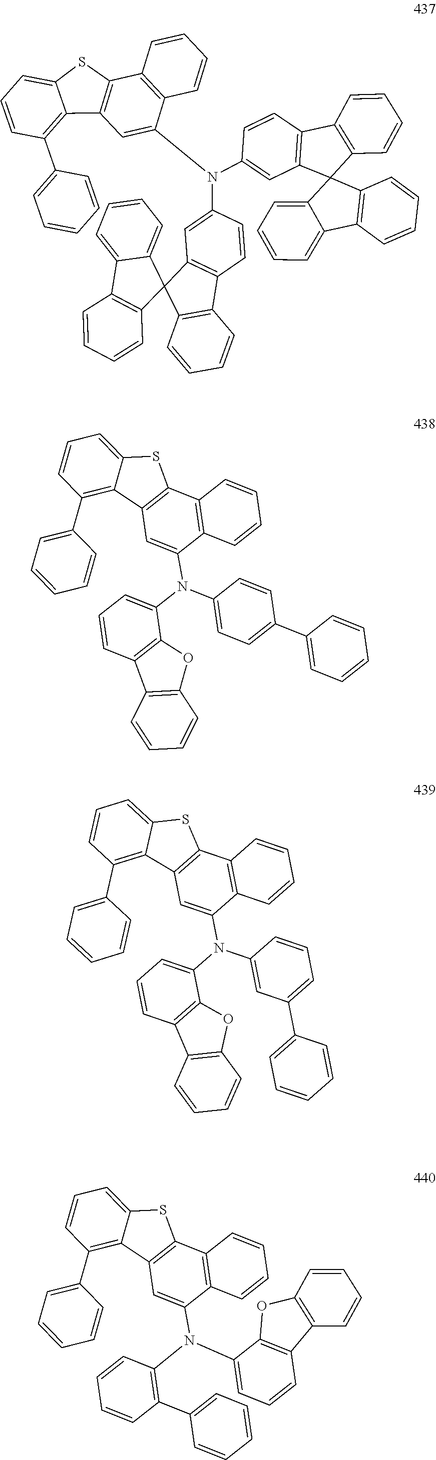 [SMILES: c1ccc(-c2cccc3sc4c5ccccc5c(N(c5ccc6c(c5)C5(c7ccccc7-c7ccccc75)c5ccccc5-6)c5ccc6c(c5)C5(c7ccccc7-c7ccccc75)c5ccccc5-6)cc4c23)cc1 ; c1ccc(-c2ccc(N(c3cc4c(sc5cccc(-c6ccccc6)c54)c4ccccc34)c3cccc4c3oc3ccccc34)cc2)cc1 ; c1ccc(-c2cccc(N(c3cc4c(sc5cccc(-c6ccccc6)c54)c4ccccc34)c3cccc4c3oc3ccccc34)c2)cc1 ; c1ccc(-c2ccccc2N(c2cc3c(sc4cccc(-c5ccccc5)c43)c3ccccc23)c2cccc3c2oc2ccccc23)cc1]

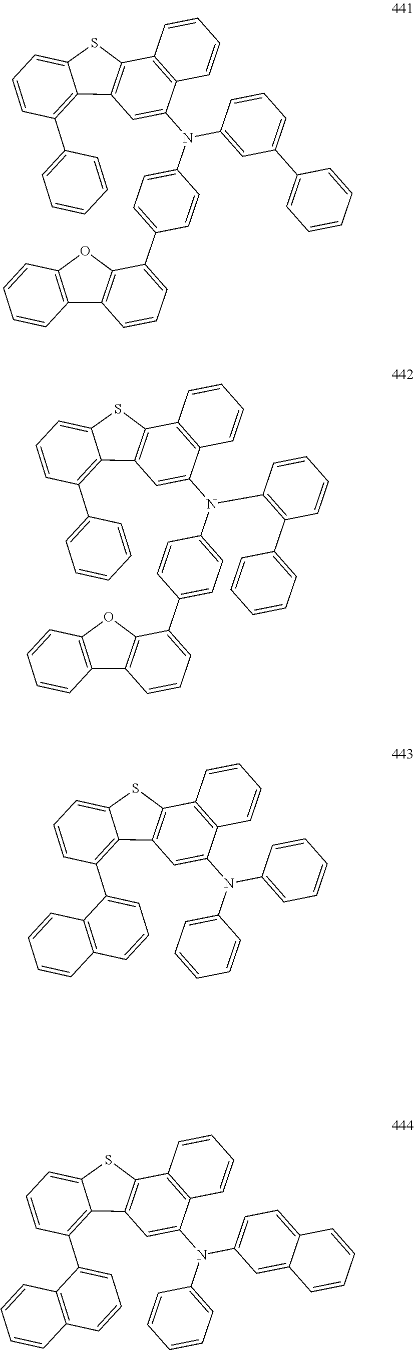 [SMILES: c1ccc(-c2cccc(N(c3ccc(-c4cccc5c4oc4ccccc45)cc3)c3cc4c(sc5cccc(-c6ccccc6)c54)c4ccccc34)c2)cc1 ; c1ccc(-c2ccccc2N(c2ccc(-c3cccc4c3oc3ccccc34)cc2)c2cc3c(sc4cccc(-c5ccccc5)c43)c3ccccc23)cc1 ; c1ccc(N(c2ccccc2)c2cc3c(sc4cccc(-c5cccc6ccccc56)c43)c3ccccc23)cc1 ; c1ccc(N(c2ccc3ccccc3c2)c2cc3c(sc4cccc(-c5cccc6ccccc56)c43)c3ccccc23)cc1]

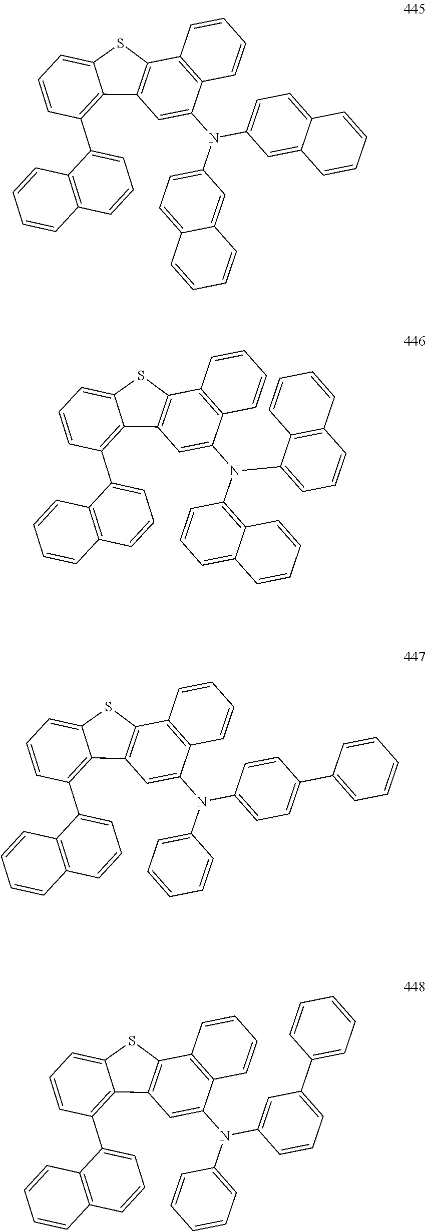 [SMILES: c1ccc2cc(N(c3ccc4ccccc4c3)c3cc4c(sc5cccc(-c6cccc7ccccc67)c54)c4ccccc34)ccc2c1 ; c1ccc2c(-c3cccc4sc5c6ccccc6c(N(c6cccc7ccccc67)c6cccc7ccccc67)cc5c34)cccc2c1 ; c1ccc(-c2ccc(N(c3ccccc3)c3cc4c(sc5cccc(-c6cccc7ccccc67)c54)c4ccccc34)cc2)cc1 ; c1ccc(-c2cccc(N(c3ccccc3)c3cc4c(sc5cccc(-c6cccc7ccccc67)c54)c4ccccc34)c2)cc1]

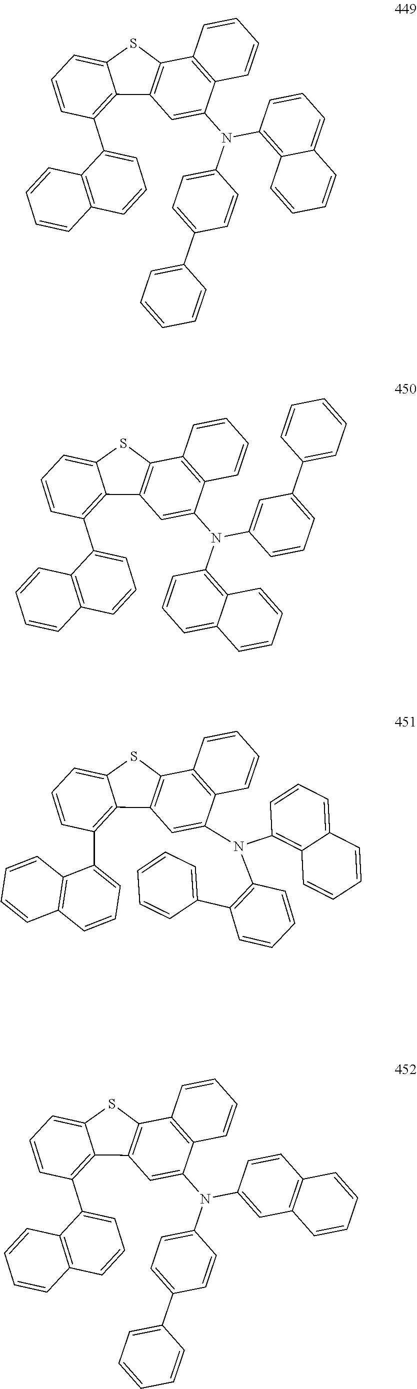 [SMILES: c1ccc(-c2ccc(N(c3cccc4ccccc34)c3cc4c(sc5cccc(-c6cccc7ccccc67)c54)c4ccccc34)cc2)cc1 ; c1ccc(-c2cccc(N(c3cccc4ccccc34)c3cc4c(sc5cccc(-c6cccc7ccccc67)c54)c4ccccc34)c2)cc1 ; c1ccc(-c2ccccc2N(c2cccc3ccccc23)c2cc3c(sc4cccc(-c5cccc6ccccc56)c43)c3ccccc23)cc1 ; c1ccc(-c2ccc(N(c3ccc4ccccc4c3)c3cc4c(sc5cccc(-c6cccc7ccccc67)c54)c4ccccc34)cc2)cc1]

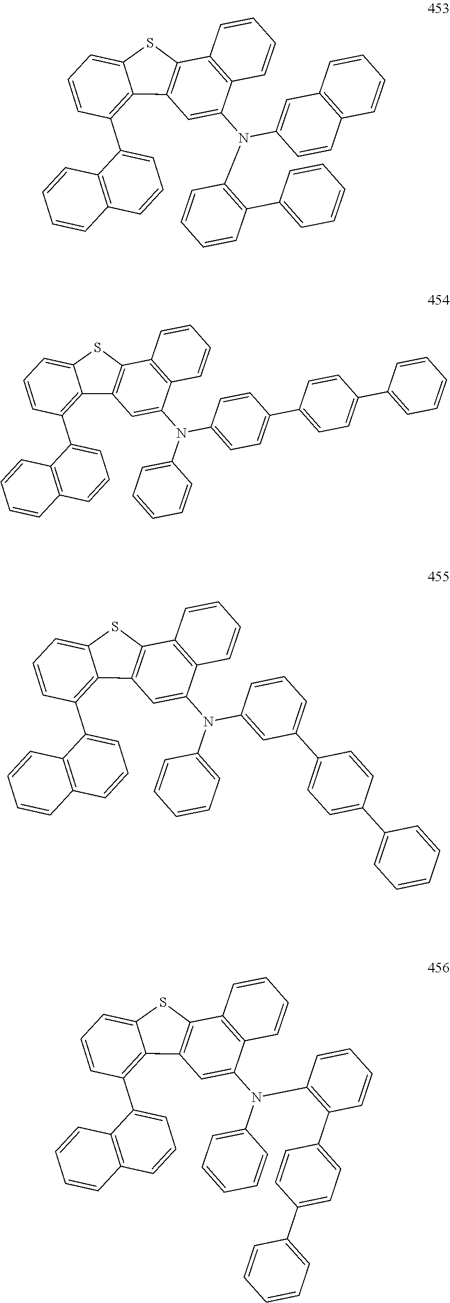 [SMILES: c1ccc(-c2ccccc2N(c2ccc3ccccc3c2)c2cc3c(sc4cccc(-c5cccc6ccccc56)c43)c3ccccc23)cc1 ; c1ccc(-c2ccc(-c3ccc(N(c4ccccc4)c4cc5c(sc6cccc(-c7cccc8ccccc78)c65)c5ccccc45)cc3)cc2)cc1 ; c1ccc(-c2ccc(-c3cccc(N(c4ccccc4)c4cc5c(sc6cccc(-c7cccc8ccccc78)c65)c5ccccc45)c3)cc2)cc1 ; c1ccc(-c2ccc(-c3ccccc3N(c3ccccc3)c3cc4c(sc5cccc(-c6cccc7ccccc67)c54)c4ccccc34)cc2)cc1]

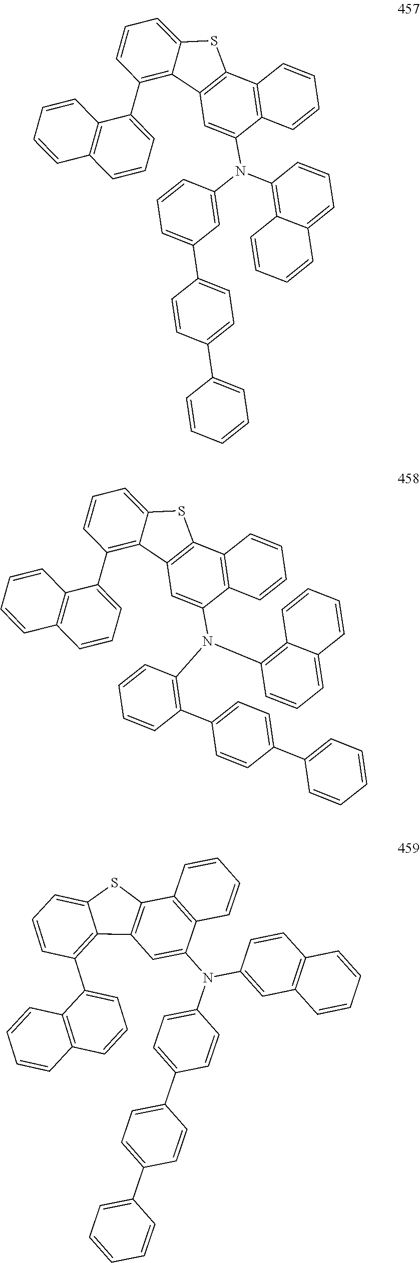 [SMILES: c1ccc(-c2ccc(-c3cccc(N(c4cccc5ccccc45)c4cc5c(sc6cccc(-c7cccc8ccccc78)c65)c5ccccc45)c3)cc2)cc1 ; c1ccc(-c2ccc(-c3ccccc3N(c3cccc4ccccc34)c3cc4c(sc5cccc(-c6cccc7ccccc67)c54)c4ccccc34)cc2)cc1 ; c1ccc(-c2ccc(-c3ccc(N(c4ccc5ccccc5c4)c4cc5c(sc6cccc(-c7cccc8ccccc78)c65)c5ccccc45)cc3)cc2)cc1]

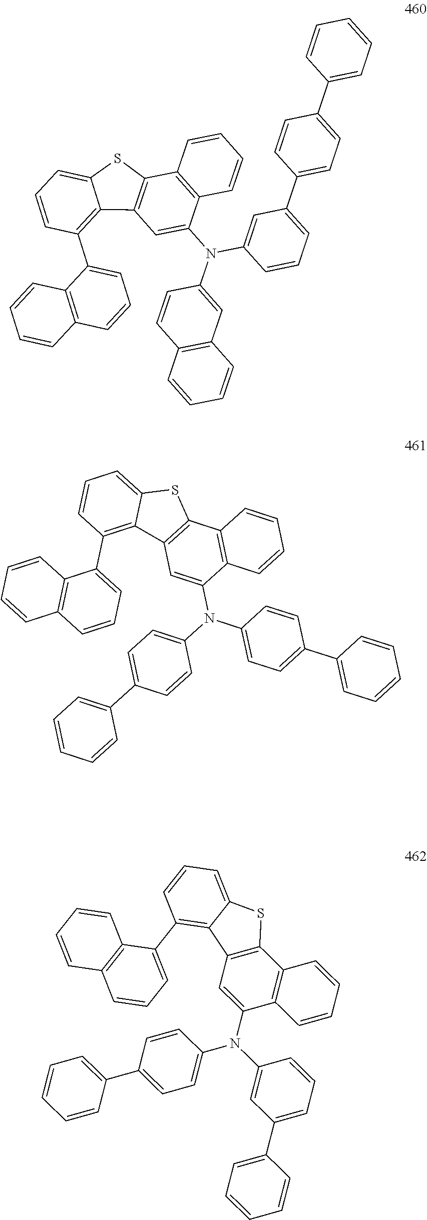 [SMILES: c1ccc(-c2ccc(-c3cccc(N(c4ccc5ccccc5c4)c4cc5c(sc6cccc(-c7cccc8ccccc78)c65)c5ccccc45)c3)cc2)cc1 ; c1ccc(-c2ccc(N(c3ccc(-c4ccccc4)cc3)c3cc4c(sc5cccc(-c6cccc7ccccc67)c54)c4ccccc34)cc2)cc1 ; c1ccc(-c2ccc(N(c3cccc(-c4ccccc4)c3)c3cc4c(sc5cccc(-c6cccc7ccccc67)c54)c4ccccc34)cc2)cc1]

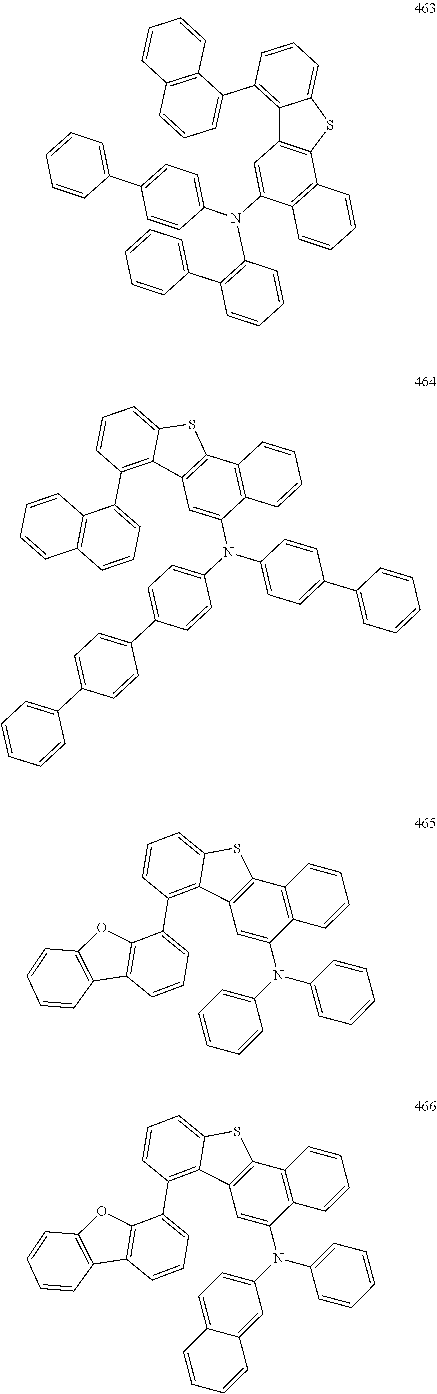 [SMILES: c1ccc(-c2ccc(N(c3ccccc3-c3ccccc3)c3cc4c(sc5cccc(-c6cccc7ccccc67)c54)c4ccccc34)cc2)cc1 ; c1ccc(-c2ccc(-c3ccc(N(c4ccc(-c5ccccc5)cc4)c4cc5c(sc6cccc(-c7cccc8ccccc78)c65)c5ccccc45)cc3)cc2)cc1 ; c1ccc(N(c2ccccc2)c2cc3c(sc4cccc(-c5cccc6c5oc5ccccc56)c43)c3ccccc23)cc1 ; c1ccc(N(c2ccc3ccccc3c2)c2cc3c(sc4cccc(-c5cccc6c5oc5ccccc56)c43)c3ccccc23)cc1]

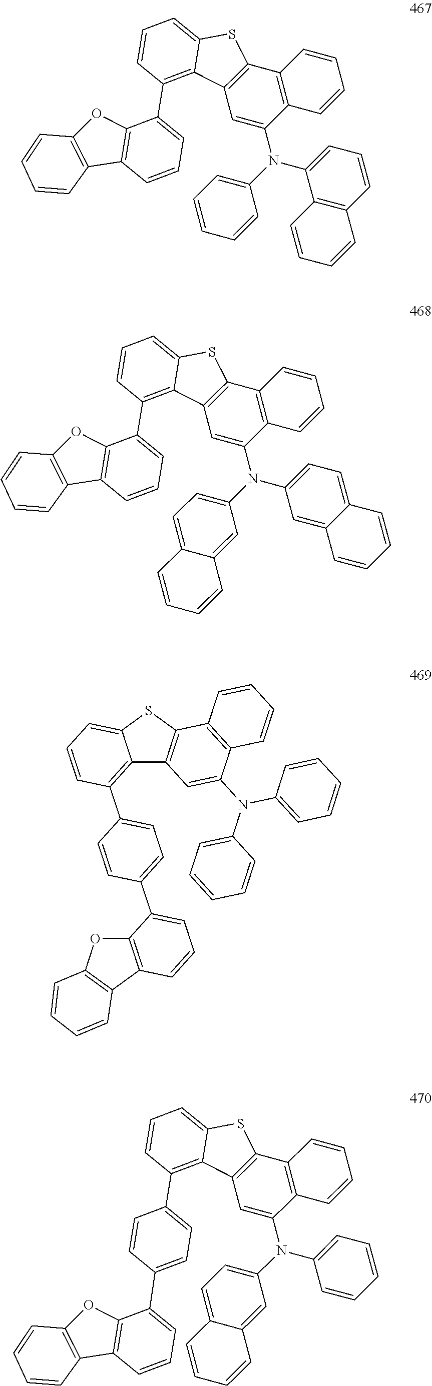 [SMILES: c1ccc(N(c2cccc3ccccc23)c2cc3c(sc4cccc(-c5cccc6c5oc5ccccc56)c43)c3ccccc23)cc1 ; c1ccc2cc(N(c3ccc4ccccc4c3)c3cc4c(sc5cccc(-c6cccc7c6oc6ccccc67)c54)c4ccccc34)ccc2c1 ; c1ccc(N(c2ccccc2)c2cc3c(sc4cccc(-c5ccc(-c6cccc7c6oc6ccccc67)cc5)c43)c3ccccc23)cc1 ; c1ccc(N(c2ccc3ccccc3c2)c2cc3c(sc4cccc(-c5ccc(-c6cccc7c6oc6ccccc67)cc5)c43)c3ccccc23)cc1]

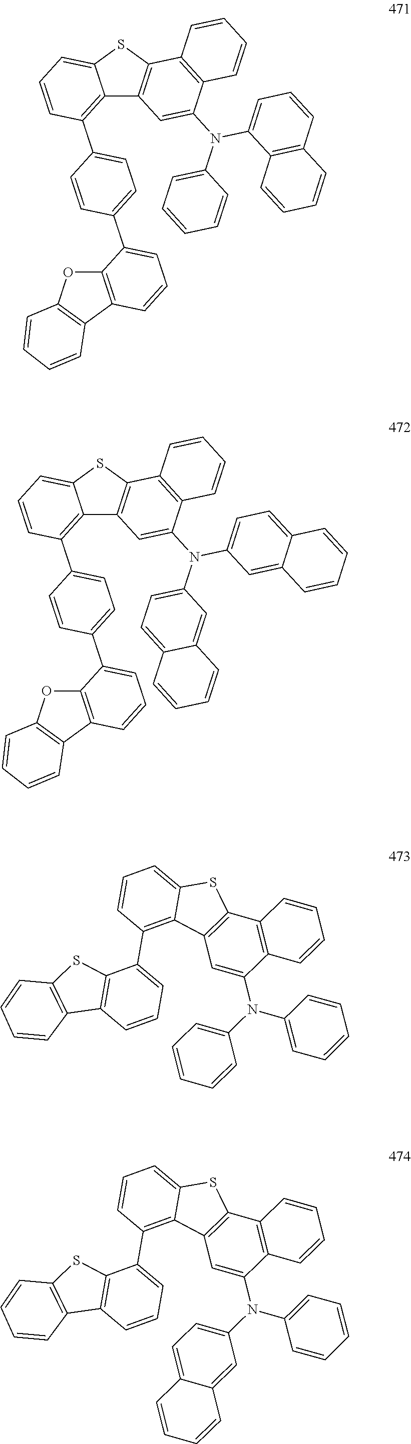 [SMILES: c1ccc(N(c2cccc3ccccc23)c2cc3c(sc4cccc(-c5ccc(-c6cccc7c6oc6ccccc67)cc5)c43)c3ccccc23)cc1 ; c1ccc2cc(N(c3ccc4ccccc4c3)c3cc4c(sc5cccc(-c6ccc(-c7cccc8c7oc7ccccc78)cc6)c54)c4ccccc34)ccc2c1 ; c1ccc(N(c2ccccc2)c2cc3c(sc4cccc(-c5cccc6c5sc5ccccc56)c43)c3ccccc23)cc1 ; c1ccc(N(c2ccc3ccccc3c2)c2cc3c(sc4cccc(-c5cccc6c5sc5ccccc56)c43)c3ccccc23)cc1]

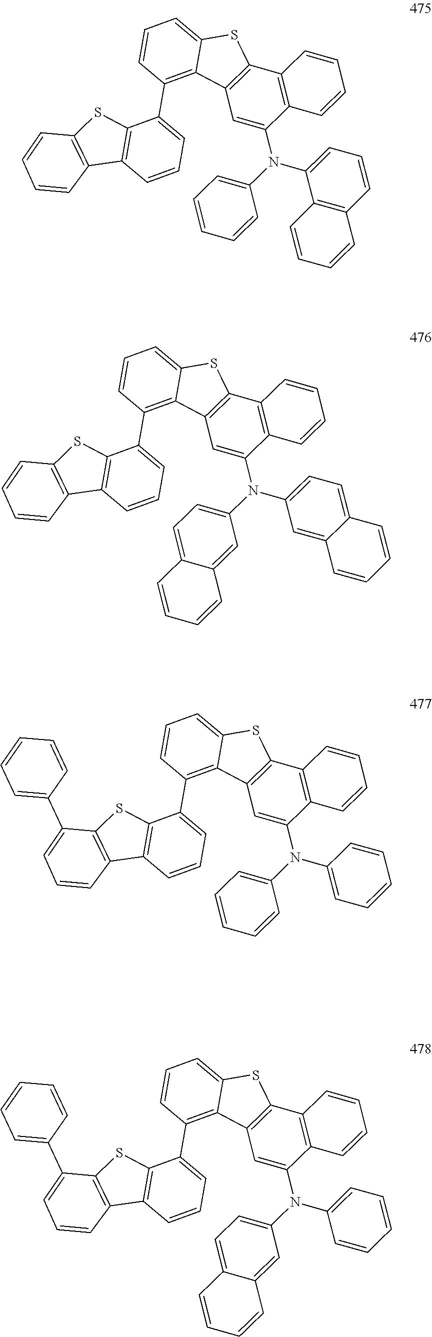 [SMILES: c1ccc(N(c2cccc3ccccc23)c2cc3c(sc4cccc(-c5cccc6c5sc5ccccc56)c43)c3ccccc23)cc1 ; c1ccc2cc(N(c3ccc4ccccc4c3)c3cc4c(sc5cccc(-c6cccc7c6sc6ccccc67)c54)c4ccccc34)ccc2c1 ; c1ccc(-c2cccc3c2sc2c(-c4cccc5sc6c7ccccc7c(N(c7ccccc7)c7ccccc7)cc6c45)cccc23)cc1 ; c1ccc(-c2cccc3c2sc2c(-c4cccc5sc6c7ccccc7c(N(c7ccccc7)c7ccc8ccccc8c7)cc6c45)cccc23)cc1]

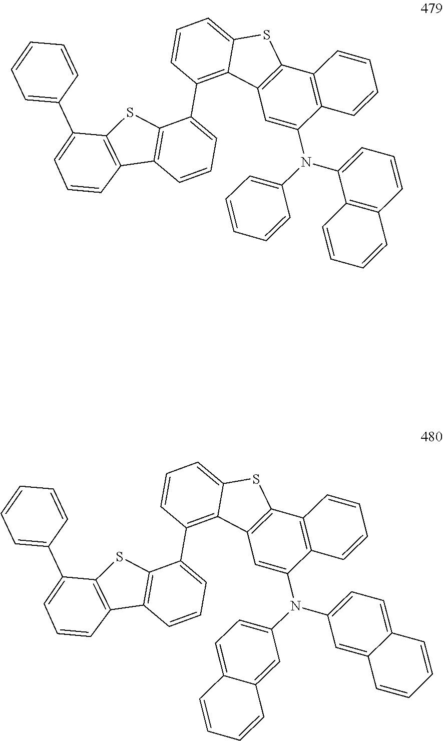 [SMILES: c1ccc(-c2cccc3c2sc2c(-c4cccc5sc6c7ccccc7c(N(c7ccccc7)c7cccc8ccccc78)cc6c45)cccc23)cc1 ; c1ccc(-c2cccc3c2sc2c(-c4cccc5sc6c7ccccc7c(N(c7ccc8ccccc8c7)c7ccc8ccccc8c7)cc6c45)cccc23)cc1]

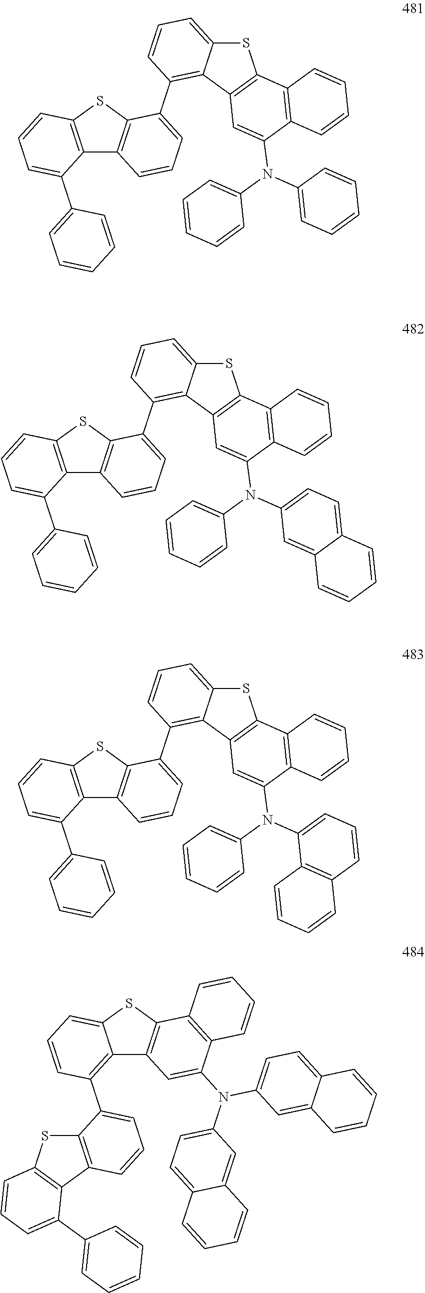 [SMILES: c1ccc(-c2cccc3sc4c(-c5cccc6sc7c8ccccc8c(N(c8ccccc8)c8ccccc8)cc7c56)cccc4c23)cc1 ; c1ccc(-c2cccc3sc4c(-c5cccc6sc7c8ccccc8c(N(c8ccccc8)c8ccc9ccccc9c8)cc7c56)cccc4c23)cc1 ; c1ccc(-c2cccc3sc4c(-c5cccc6sc7c8ccccc8c(N(c8ccccc8)c8cccc9ccccc89)cc7c56)cccc4c23)cc1 ; c1ccc(-c2cccc3sc4c(-c5cccc6sc7c8ccccc8c(N(c8ccc9ccccc9c8)c8ccc9ccccc9c8)cc7c56)cccc4c23)cc1]

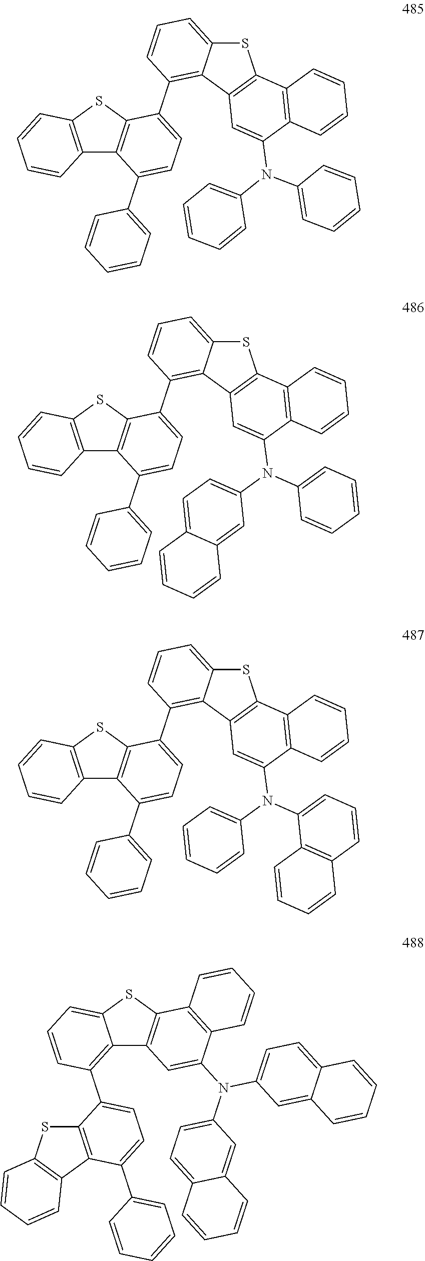 [SMILES: c1ccc(-c2ccc(-c3cccc4sc5c6ccccc6c(N(c6ccccc6)c6ccccc6)cc5c34)c3sc4ccccc4c23)cc1 ; c1ccc(-c2ccc(-c3cccc4sc5c6ccccc6c(N(c6ccccc6)c6ccc7ccccc7c6)cc5c34)c3sc4ccccc4c23)cc1 ; c1ccc(-c2ccc(-c3cccc4sc5c6ccccc6c(N(c6ccccc6)c6cccc7ccccc67)cc5c34)c3sc4ccccc4c23)cc1 ; c1ccc(-c2ccc(-c3cccc4sc5c6ccccc6c(N(c6ccc7ccccc7c6)c6ccc7ccccc7c6)cc5c34)c3sc4ccccc4c23)cc1]

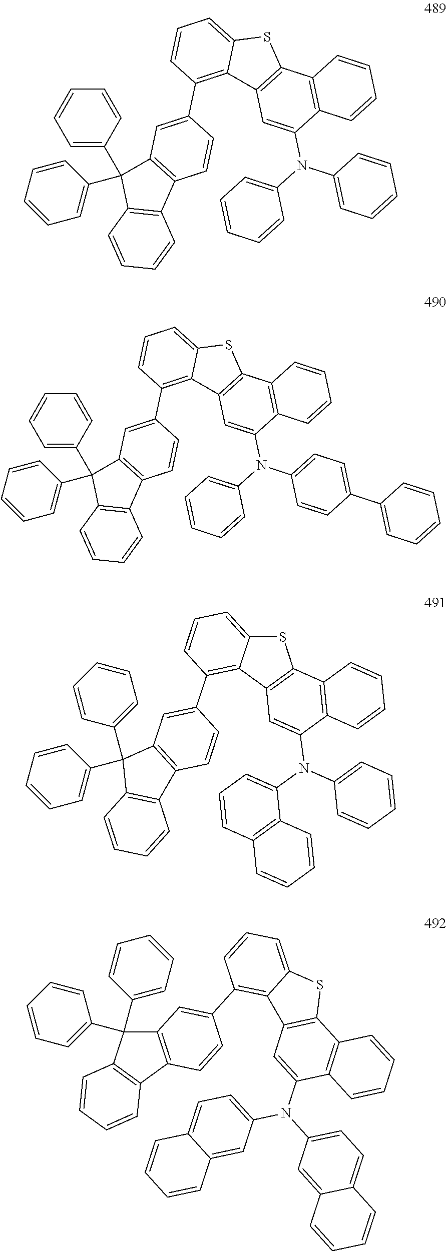 [SMILES: c1ccc(N(c2ccccc2)c2cc3c(sc4cccc(-c5ccc6c(c5)C(c5ccccc5)(c5ccccc5)c5ccccc5-6)c43)c3ccccc23)cc1 ; c1ccc(-c2ccc(N(c3ccccc3)c3cc4c(sc5cccc(-c6ccc7c(c6)C(c6ccccc6)(c6ccccc6)c6ccccc6-7)c54)c4ccccc34)cc2)cc1 ; c1ccc(N(c2cccc3ccccc23)c2cc3c(sc4cccc(-c5ccc6c(c5)C(c5ccccc5)(c5ccccc5)c5ccccc5-6)c43)c3ccccc23)cc1 ; c1ccc(C2(c3ccccc3)c3ccccc3-c3ccc(-c4cccc5sc6c7ccccc7c(N(c7ccc8ccccc8c7)c7ccc8ccccc8c7)cc6c45)cc32)cc1]

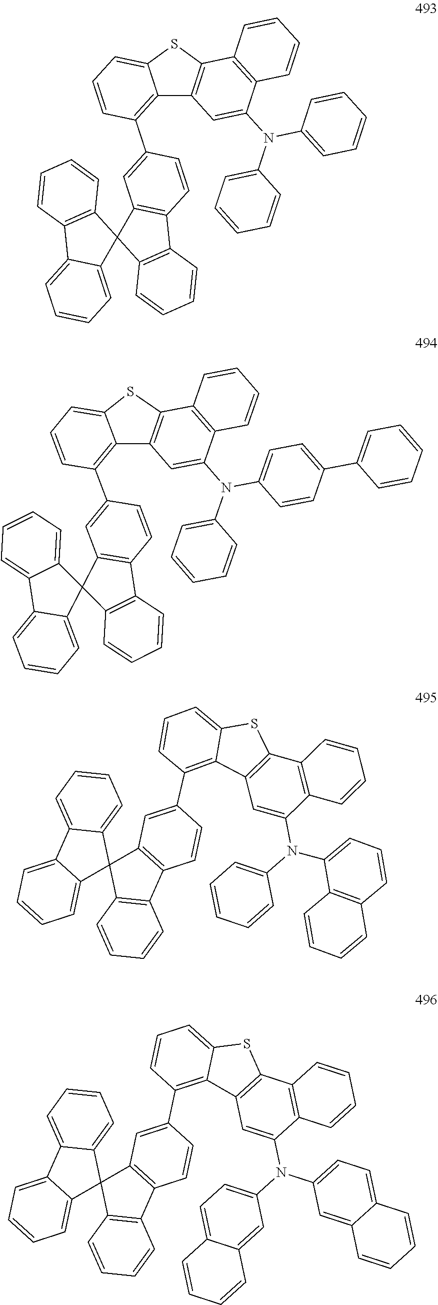 [SMILES: c1ccc(N(c2ccccc2)c2cc3c(sc4cccc(-c5ccc6c(c5)C5(c7ccccc7-c7ccccc75)c5ccccc5-6)c43)c3ccccc23)cc1 ; c1ccc(-c2ccc(N(c3ccccc3)c3cc4c(sc5cccc(-c6ccc7c(c6)C6(c8ccccc8-c8ccccc86)c6ccccc6-7)c54)c4ccccc34)cc2)cc1 ; c1ccc(N(c2cccc3ccccc23)c2cc3c(sc4cccc(-c5ccc6c(c5)C5(c7ccccc7-c7ccccc75)c5ccccc5-6)c43)c3ccccc23)cc1 ; c1ccc2c(c1)-c1ccccc1C21c2ccccc2-c2ccc(-c3cccc4sc5c6ccccc6c(N(c6ccc7ccccc7c6)c6ccc7ccccc7c6)cc5c34)cc21]

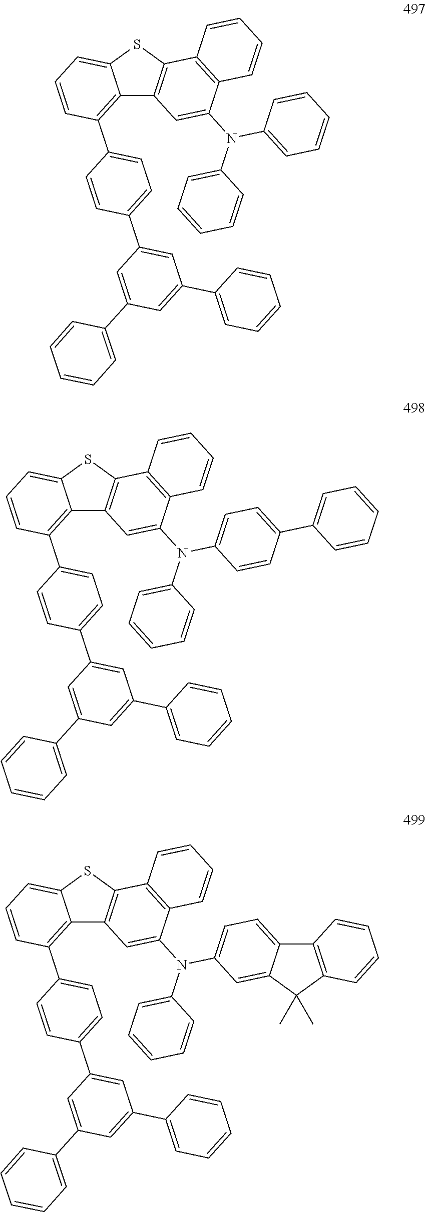 [SMILES: c1ccc(-c2cc(-c3ccccc3)cc(-c3ccc(-c4cccc5sc6c7ccccc7c(N(c7ccccc7)c7ccccc7)cc6c45)cc3)c2)cc1 ; c1ccc(-c2ccc(N(c3ccccc3)c3cc4c(sc5cccc(-c6ccc(-c7cc(-c8ccccc8)cc(-c8ccccc8)c7)cc6)c54)c4ccccc34)cc2)cc1 ; CC1(C)c2ccccc2-c2ccc(N(c3ccccc3)c3cc4c(sc5cccc(-c6ccc(-c7cc(-c8ccccc8)cc(-c8ccccc8)c7)cc6)c54)c4ccccc34)cc21]

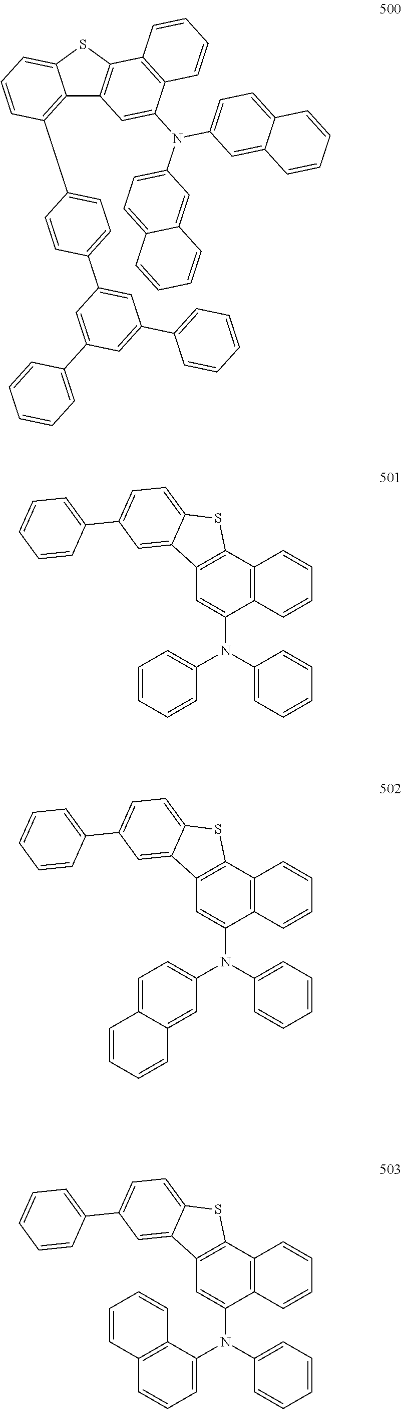 [SMILES: c1ccc(-c2cc(-c3ccccc3)cc(-c3ccc(-c4cccc5sc6c7ccccc7c(N(c7ccc8ccccc8c7)c7ccc8ccccc8c7)cc6c45)cc3)c2)cc1 ; c1ccc(-c2ccc3sc4c5ccccc5c(N(c5ccccc5)c5ccccc5)cc4c3c2)cc1 ; c1ccc(-c2ccc3sc4c5ccccc5c(N(c5ccccc5)c5ccc6ccccc6c5)cc4c3c2)cc1 ; c1ccc(-c2ccc3sc4c5ccccc5c(N(c5ccccc5)c5cccc6ccccc56)cc4c3c2)cc1]

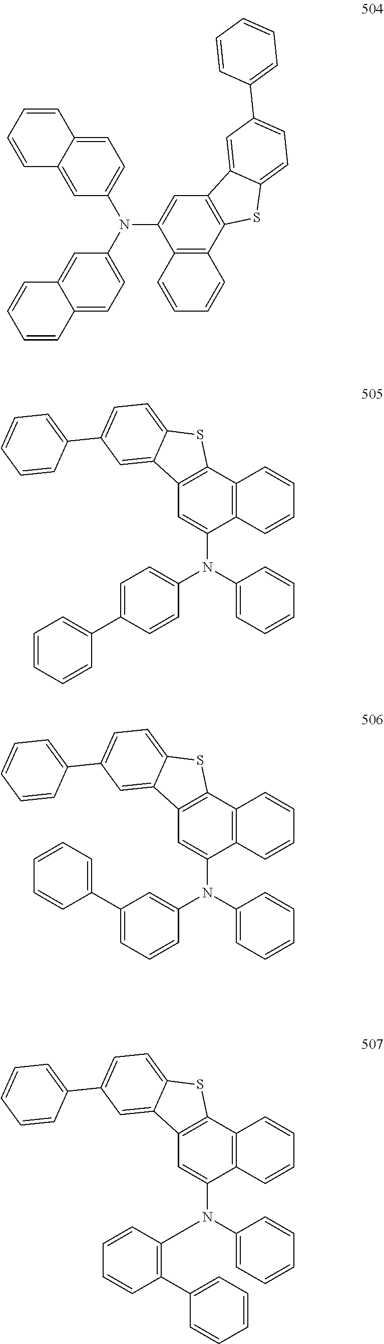 [SMILES: c1ccc(-c2ccc3sc4c5ccccc5c(N(c5ccc6ccccc6c5)c5ccc6ccccc6c5)cc4c3c2)cc1 ; c1ccc(-c2ccc(N(c3ccccc3)c3cc4c5cc(-c6ccccc6)ccc5sc4c4ccccc34)cc2)cc1 ; c1ccc(-c2cccc(N(c3ccccc3)c3cc4c5cc(-c6ccccc6)ccc5sc4c4ccccc34)c2)cc1 ; c1ccc(-c2ccc3sc4c5ccccc5c(N(c5ccccc5)c5ccccc5-c5ccccc5)cc4c3c2)cc1]

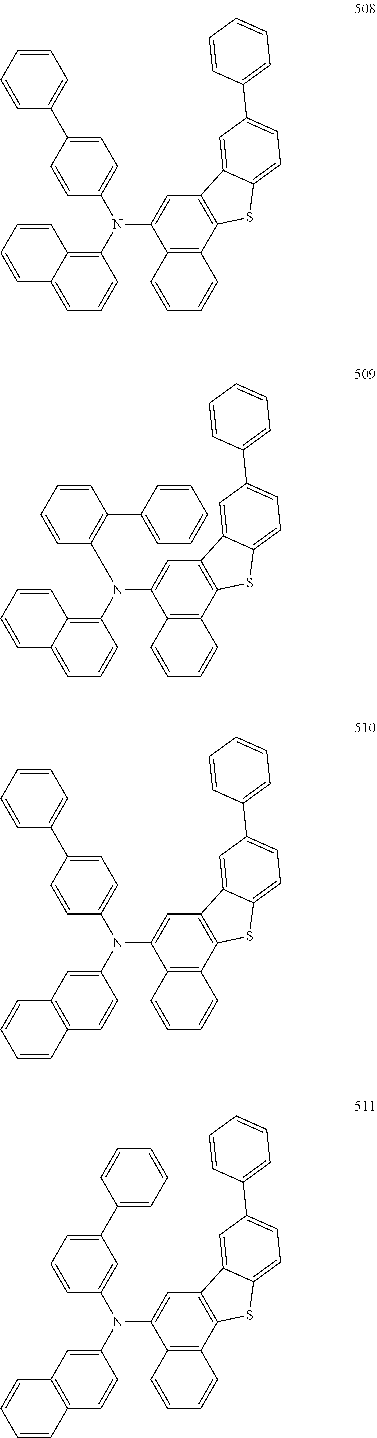 [SMILES: c1ccc(-c2ccc(N(c3cccc4ccccc34)c3cc4c5cc(-c6ccccc6)ccc5sc4c4ccccc34)cc2)cc1 ; c1ccc(-c2ccc3sc4c5ccccc5c(N(c5ccccc5-c5ccccc5)c5cccc6ccccc56)cc4c3c2)cc1 ; c1ccc(-c2ccc(N(c3ccc4ccccc4c3)c3cc4c5cc(-c6ccccc6)ccc5sc4c4ccccc34)cc2)cc1 ; c1ccc(-c2cccc(N(c3ccc4ccccc4c3)c3cc4c5cc(-c6ccccc6)ccc5sc4c4ccccc34)c2)cc1]

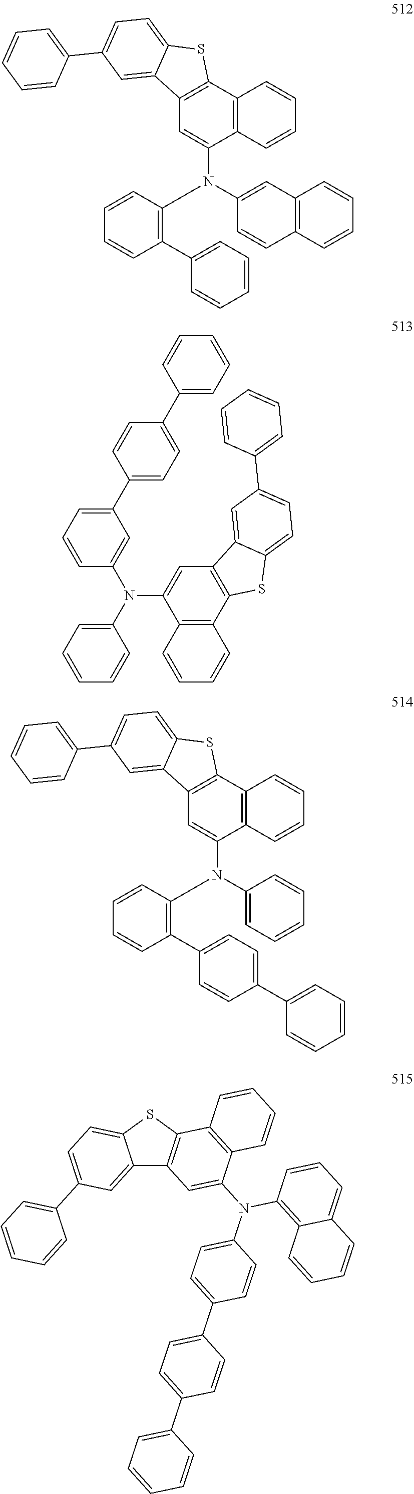

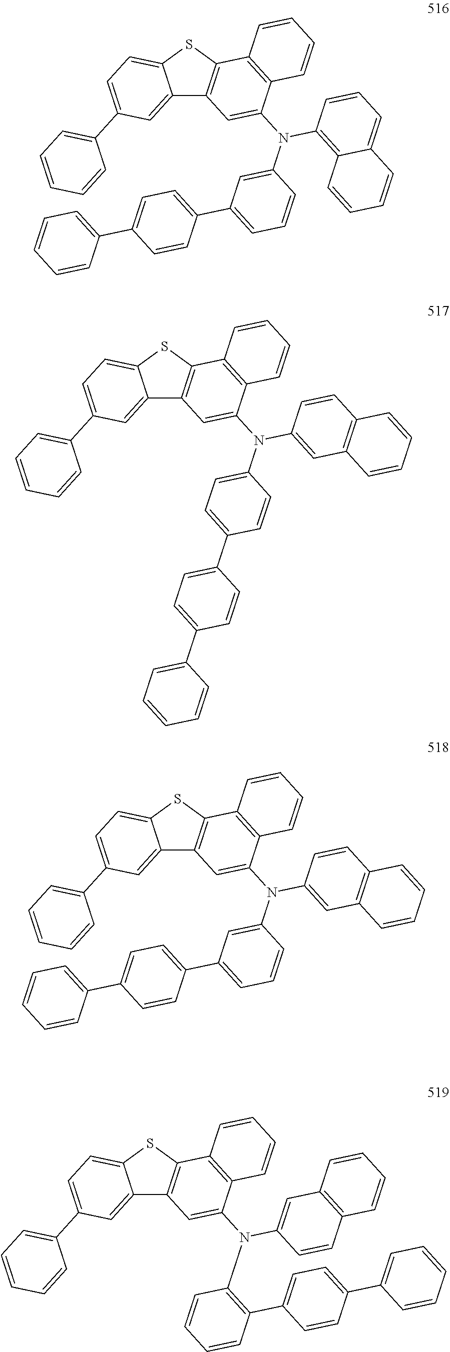 [SMILES: c1ccc(-c2ccc(-c3cccc(N(c4cccc5ccccc45)c4cc5c6cc(-c7ccccc7)ccc6sc5c5ccccc45)c3)cc2)cc1 ; c1ccc(-c2ccc(-c3ccc(N(c4ccc5ccccc5c4)c4cc5c6cc(-c7ccccc7)ccc6sc5c5ccccc45)cc3)cc2)cc1 ; c1ccc(-c2ccc(-c3cccc(N(c4ccc5ccccc5c4)c4cc5c6cc(-c7ccccc7)ccc6sc5c5ccccc45)c3)cc2)cc1 ; c1ccc(-c2ccc(-c3ccccc3N(c3ccc4ccccc4c3)c3cc4c5cc(-c6ccccc6)ccc5sc4c4ccccc34)cc2)cc1]

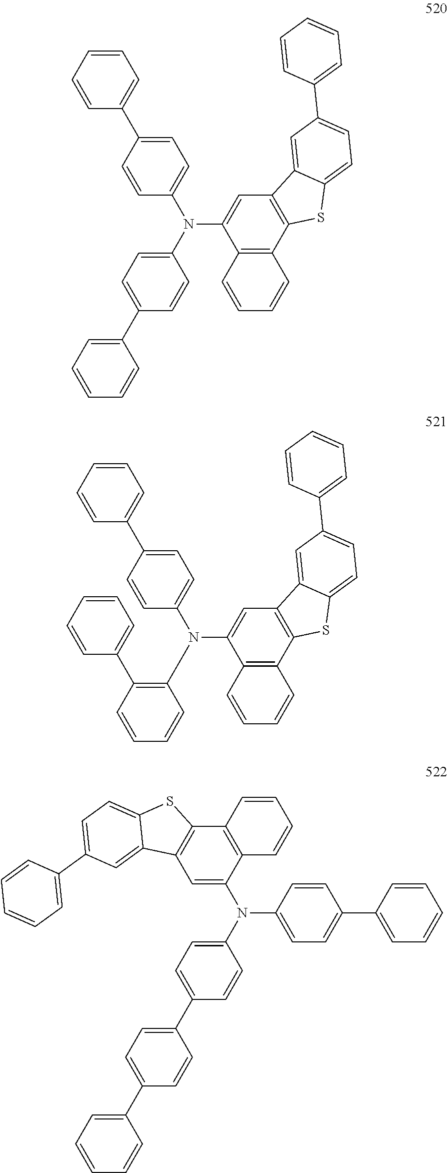 [SMILES: c1ccc(-c2ccc(N(c3ccc(-c4ccccc4)cc3)c3cc4c5cc(-c6ccccc6)ccc5sc4c4ccccc34)cc2)cc1 ; c1ccc(-c2ccc(N(c3ccccc3-c3ccccc3)c3cc4c5cc(-c6ccccc6)ccc5sc4c4ccccc34)cc2)cc1 ; c1ccc(-c2ccc(-c3ccc(N(c4ccc(-c5ccccc5)cc4)c4cc5c6cc(-c7ccccc7)ccc6sc5c5ccccc45)cc3)cc2)cc1]

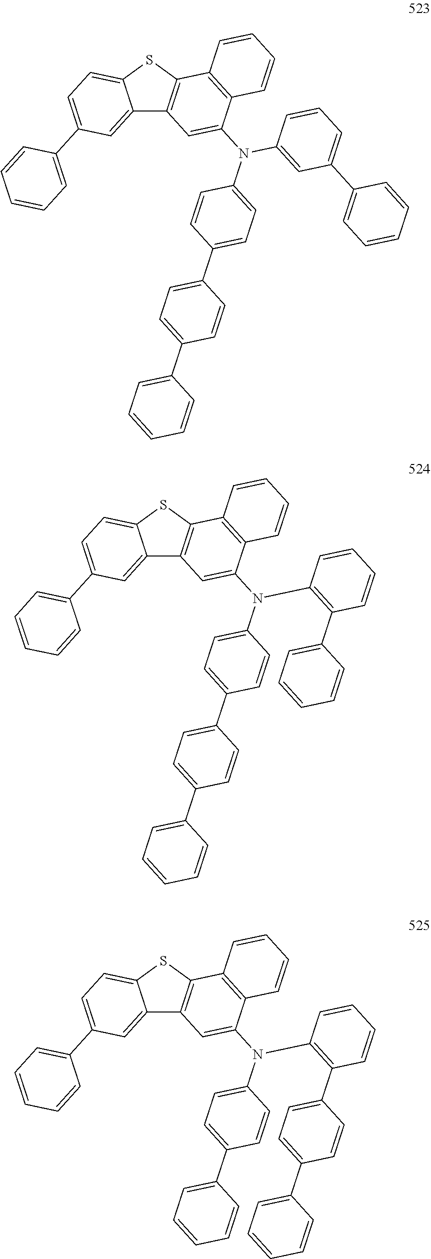 [SMILES: c1ccc(-c2ccc(-c3ccc(N(c4cccc(-c5ccccc5)c4)c4cc5c6cc(-c7ccccc7)ccc6sc5c5ccccc45)cc3)cc2)cc1 ; c1ccc(-c2ccc(-c3ccc(N(c4ccccc4-c4ccccc4)c4cc5c6cc(-c7ccccc7)ccc6sc5c5ccccc45)cc3)cc2)cc1 ; c1ccc(-c2ccc(-c3ccccc3N(c3ccc(-c4ccccc4)cc3)c3cc4c5cc(-c6ccccc6)ccc5sc4c4ccccc34)cc2)cc1]

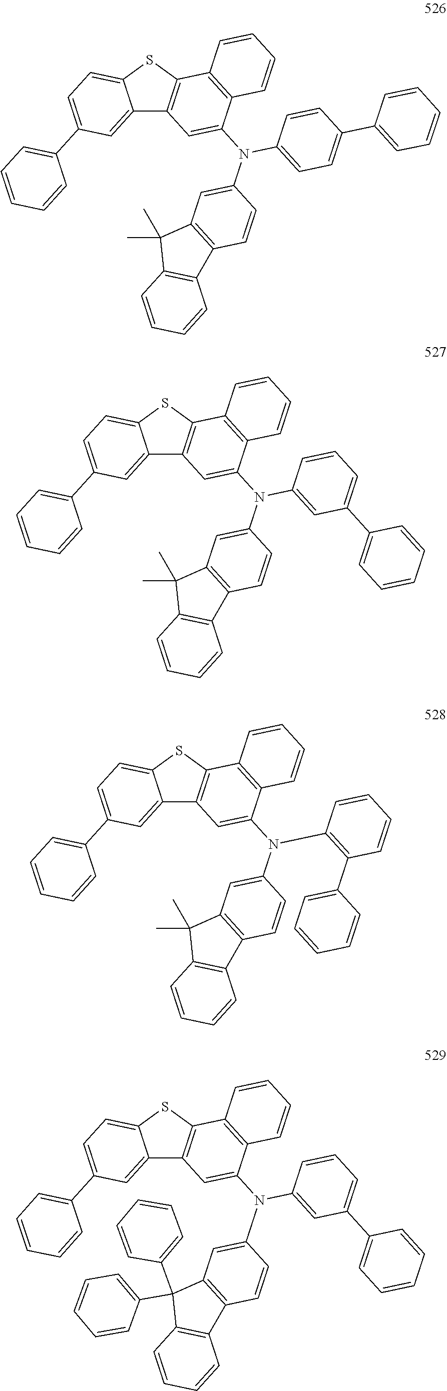 [SMILES: CC1(C)c2ccccc2-c2ccc(N(c3ccc(-c4ccccc4)cc3)c3cc4c5cc(-c6ccccc6)ccc5sc4c4ccccc34)cc21 ; CC1(C)c2ccccc2-c2ccc(N(c3cccc(-c4ccccc4)c3)c3cc4c5cc(-c6ccccc6)ccc5sc4c4ccccc34)cc21 ; CC1(C)c2ccccc2-c2ccc(N(c3ccccc3-c3ccccc3)c3cc4c5cc(-c6ccccc6)ccc5sc4c4ccccc34)cc21 ; c1ccc(-c2cccc(N(c3ccc4c(c3)C(c3ccccc3)(c3ccccc3)c3ccccc3-4)c3cc4c5cc(-c6ccccc6)ccc5sc4c4ccccc34)c2)cc1]

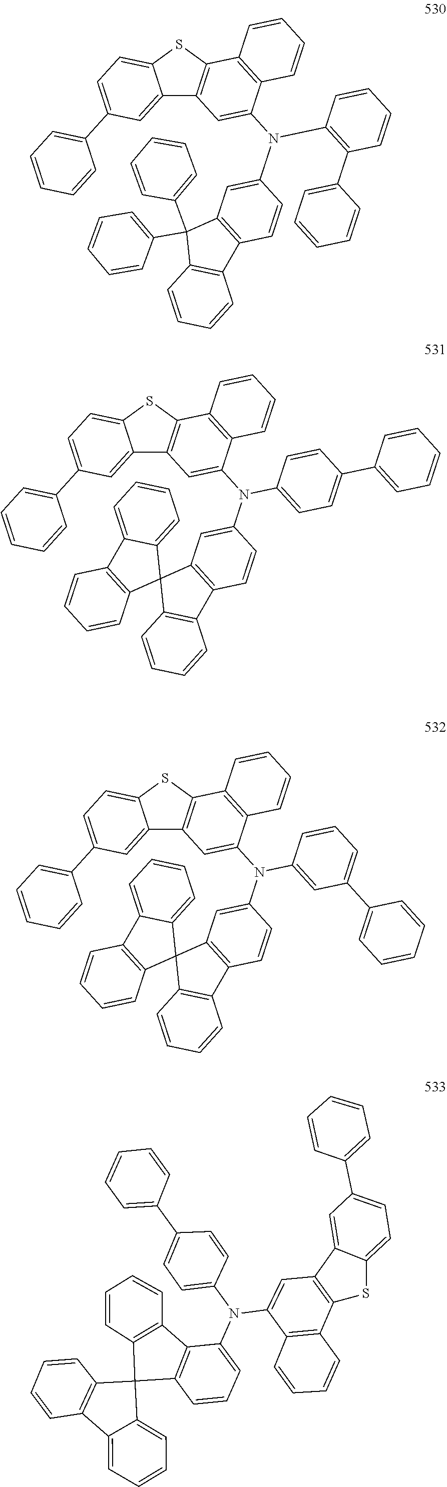 [SMILES: c1ccc(-c2ccc3sc4c5ccccc5c(N(c5ccc6c(c5)C(c5ccccc5)(c5ccccc5)c5ccccc5-6)c5ccccc5-c5ccccc5)cc4c3c2)cc1 ; c1ccc(-c2ccc(N(c3ccc4c(c3)C3(c5ccccc5-c5ccccc53)c3ccccc3-4)c3cc4c5cc(-c6ccccc6)ccc5sc4c4ccccc34)cc2)cc1 ; c1ccc(-c2cccc(N(c3ccc4c(c3)C3(c5ccccc5-c5ccccc53)c3ccccc3-4)c3cc4c5cc(-c6ccccc6)ccc5sc4c4ccccc34)c2)cc1 ; c1ccc(-c2ccc(N(c3cccc4c3-c3ccccc3C43c4ccccc4-c4ccccc43)c3cc4c5cc(-c6ccccc6)ccc5sc4c4ccccc34)cc2)cc1]

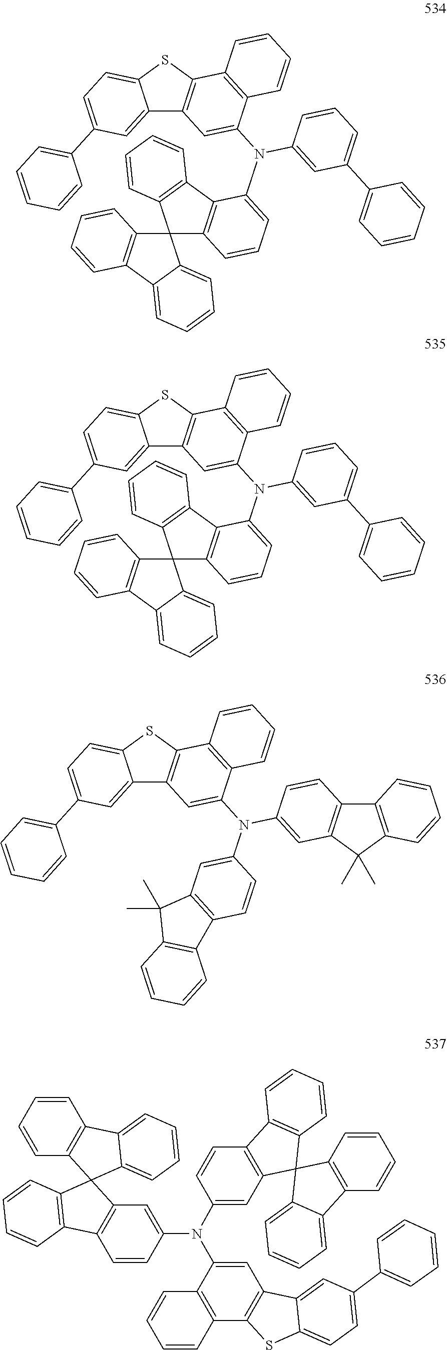 [SMILES: c1ccc(-c2cccc(N(c3cccc4c3-c3ccccc3C43c4ccccc4-c4ccccc43)c3cc4c5cc(-c6ccccc6)ccc5sc4c4ccccc34)c2)cc1 ; c1ccc(-c2cccc(N(c3cccc4c3-c3ccccc3C43c4ccccc4-c4ccccc43)c3cc4c5cc(-c6ccccc6)ccc5sc4c4ccccc34)c2)cc1 ; CC1(C)c2ccccc2-c2ccc(N(c3ccc4c(c3)C(C)(C)c3ccccc3-4)c3cc4c5cc(-c6ccccc6)ccc5sc4c4ccccc34)cc21 ; c1ccc(-c2ccc3sc4c5ccccc5c(N(c5ccc6c(c5)C5(c7ccccc7-c7ccccc75)c5ccccc5-6)c5ccc6c(c5)C5(c7ccccc7-c7ccccc75)c5ccccc5-6)cc4c3c2)cc1]

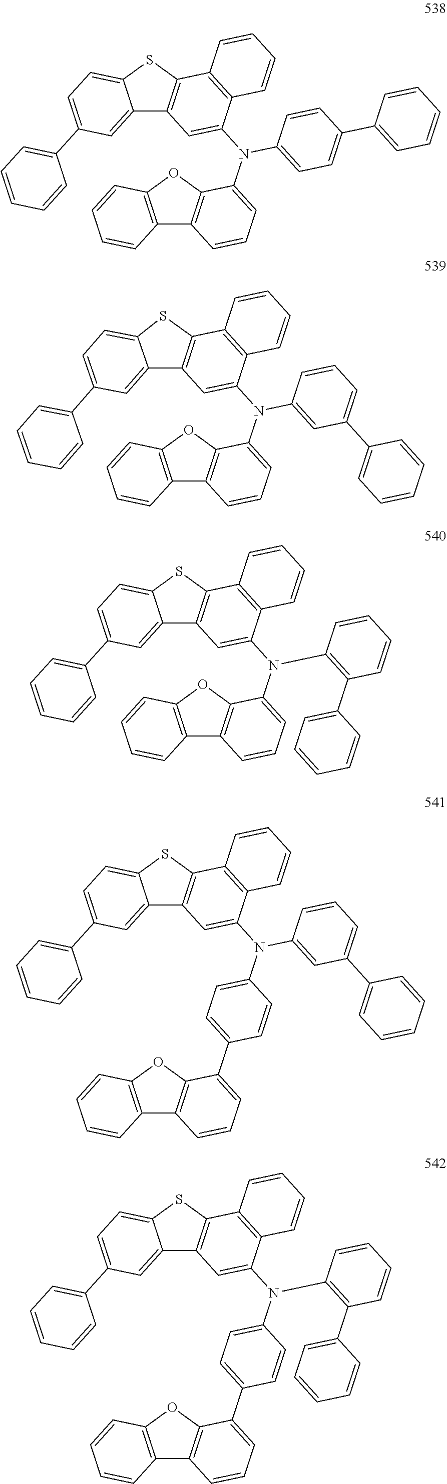 [SMILES: c1ccc(-c2ccc(N(c3cc4c5cc(-c6ccccc6)ccc5sc4c4ccccc34)c3cccc4c3oc3ccccc34)cc2)cc1 ; c1ccc(-c2cccc(N(c3cc4c5cc(-c6ccccc6)ccc5sc4c4ccccc34)c3cccc4c3oc3ccccc34)c2)cc1 ; c1ccc(-c2ccc3sc4c5ccccc5c(N(c5ccccc5-c5ccccc5)c5cccc6c5oc5ccccc56)cc4c3c2)cc1 ; c1ccc(-c2cccc(N(c3ccc(-c4cccc5c4oc4ccccc45)cc3)c3cc4c5cc(-c6ccccc6)ccc5sc4c4ccccc34)c2)cc1 ; c1ccc(-c2ccc3sc4c5ccccc5c(N(c5ccc(-c6cccc7c6oc6ccccc67)cc5)c5ccccc5-c5ccccc5)cc4c3c2)cc1]

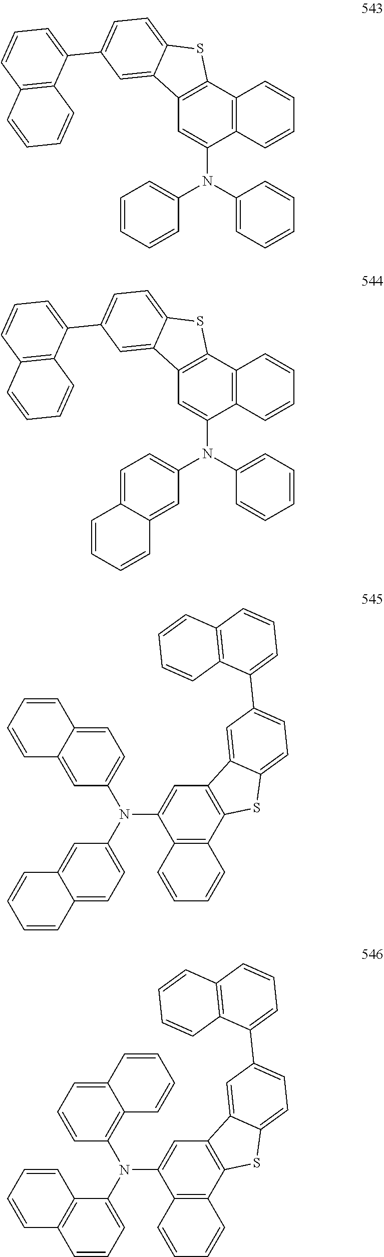

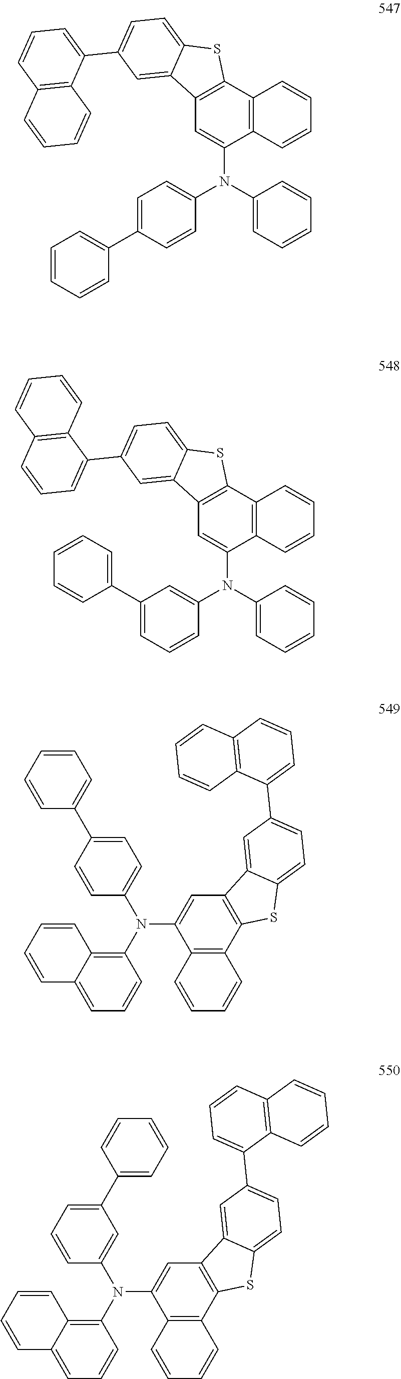 [SMILES: c1ccc(-c2ccc(N(c3ccccc3)c3cc4c5cc(-c6cccc7ccccc67)ccc5sc4c4ccccc34)cc2)cc1 ; c1ccc(-c2cccc(N(c3ccccc3)c3cc4c5cc(-c6cccc7ccccc67)ccc5sc4c4ccccc34)c2)cc1 ; c1ccc(-c2ccc(N(c3cccc4ccccc34)c3cc4c5cc(-c6cccc7ccccc67)ccc5sc4c4ccccc34)cc2)cc1 ; c1ccc(-c2cccc(N(c3cccc4ccccc34)c3cc4c5cc(-c6cccc7ccccc67)ccc5sc4c4ccccc34)c2)cc1]

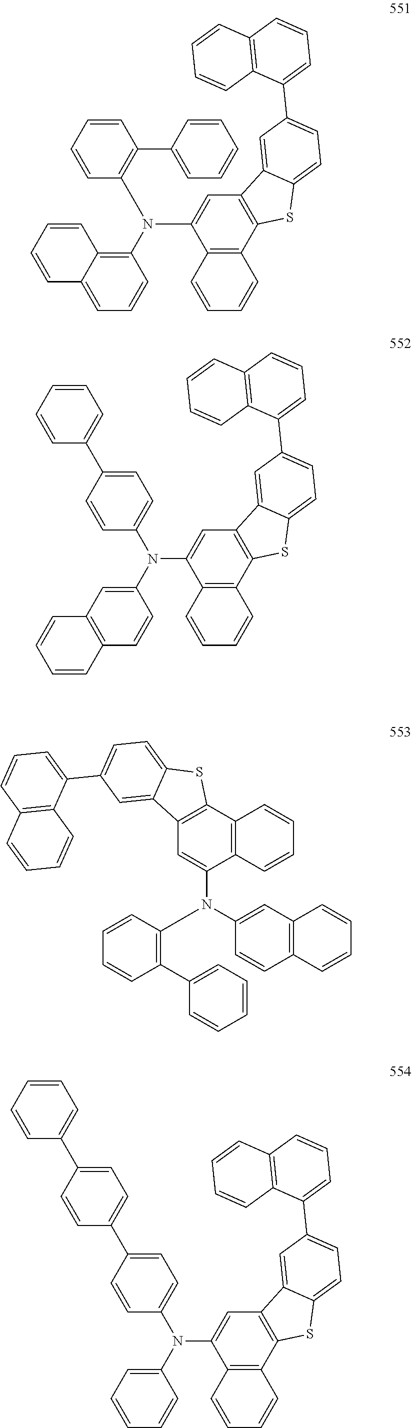 [SMILES: c1ccc(-c2ccccc2N(c2cccc3ccccc23)c2cc3c4cc(-c5cccc6ccccc56)ccc4sc3c3ccccc23)cc1 ; c1ccc(-c2ccc(N(c3ccc4ccccc4c3)c3cc4c5cc(-c6cccc7ccccc67)ccc5sc4c4ccccc34)cc2)cc1 ; c1ccc(-c2ccccc2N(c2ccc3ccccc3c2)c2cc3c4cc(-c5cccc6ccccc56)ccc4sc3c3ccccc23)cc1 ; c1ccc(-c2ccc(-c3ccc(N(c4ccccc4)c4cc5c6cc(-c7cccc8ccccc78)ccc6sc5c5ccccc45)cc3)cc2)cc1]

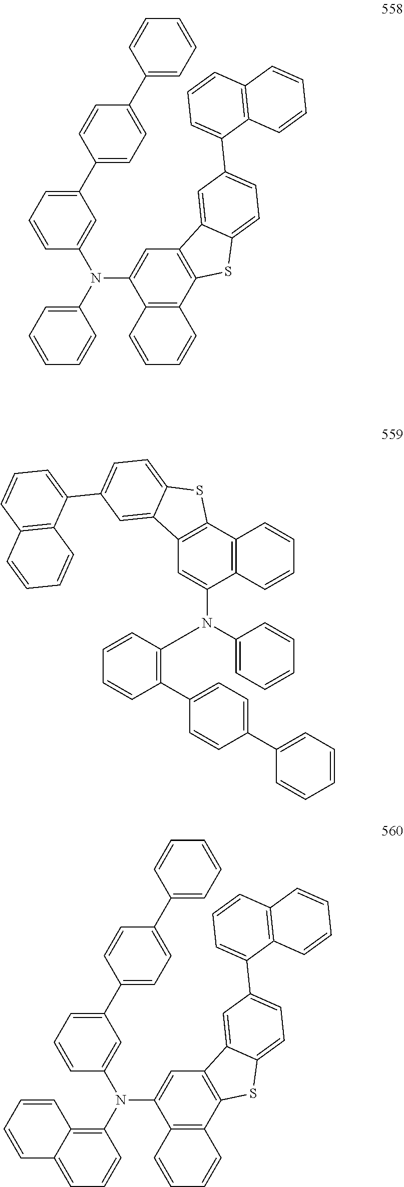 [SMILES: c1ccc(-c2ccc(-c3cccc(N(c4ccccc4)c4cc5c6cc(-c7cccc8ccccc78)ccc6sc5c5ccccc45)c3)cc2)cc1 ; c1ccc(-c2ccc(-c3ccccc3N(c3ccccc3)c3cc4c5cc(-c6cccc7ccccc67)ccc5sc4c4ccccc34)cc2)cc1 ; c1ccc(-c2ccc(-c3cccc(N(c4cccc5ccccc45)c4cc5c6cc(-c7cccc8ccccc78)ccc6sc5c5ccccc45)c3)cc2)cc1]

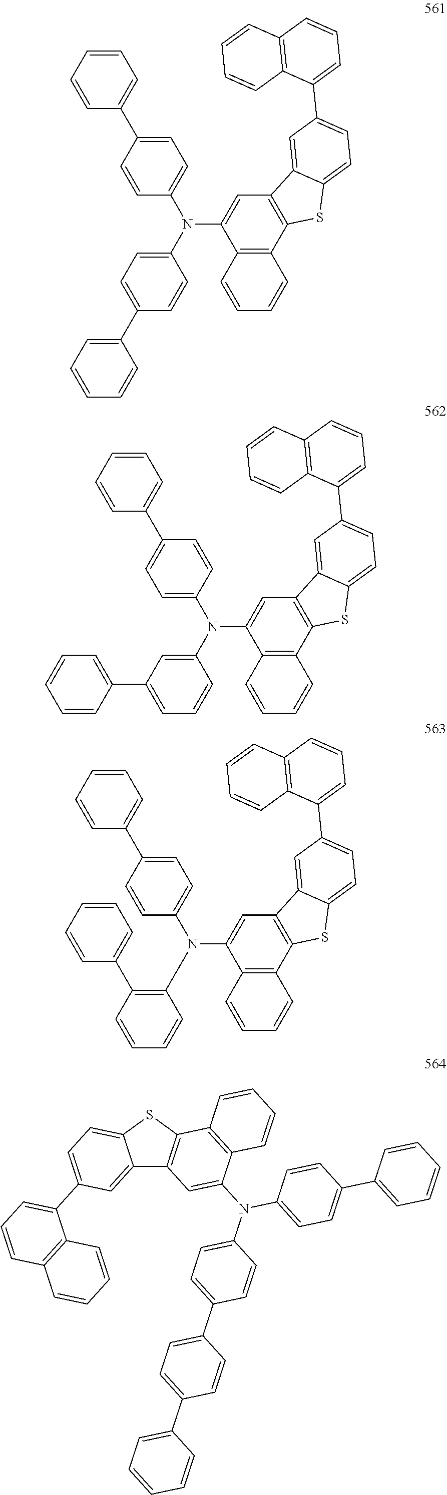

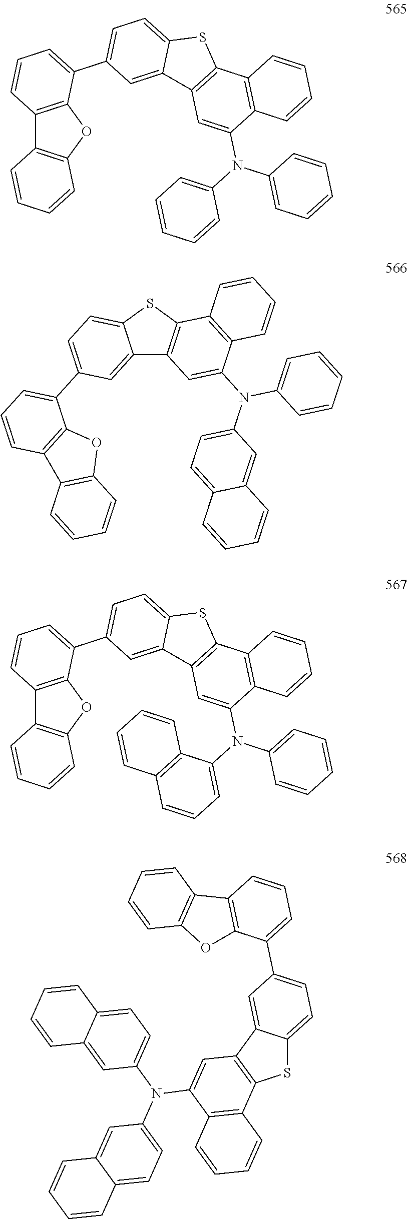 [SMILES: c1ccc(N(c2ccccc2)c2cc3c4cc(-c5cccc6c5oc5ccccc56)ccc4sc3c3ccccc23)cc1 ; c1ccc(N(c2ccc3ccccc3c2)c2cc3c4cc(-c5cccc6c5oc5ccccc56)ccc4sc3c3ccccc23)cc1 ; c1ccc(N(c2cccc3ccccc23)c2cc3c4cc(-c5cccc6c5oc5ccccc56)ccc4sc3c3ccccc23)cc1 ; c1ccc2cc(N(c3ccc4ccccc4c3)c3cc4c5cc(-c6cccc7c6oc6ccccc67)ccc5sc4c4ccccc34)ccc2c1]

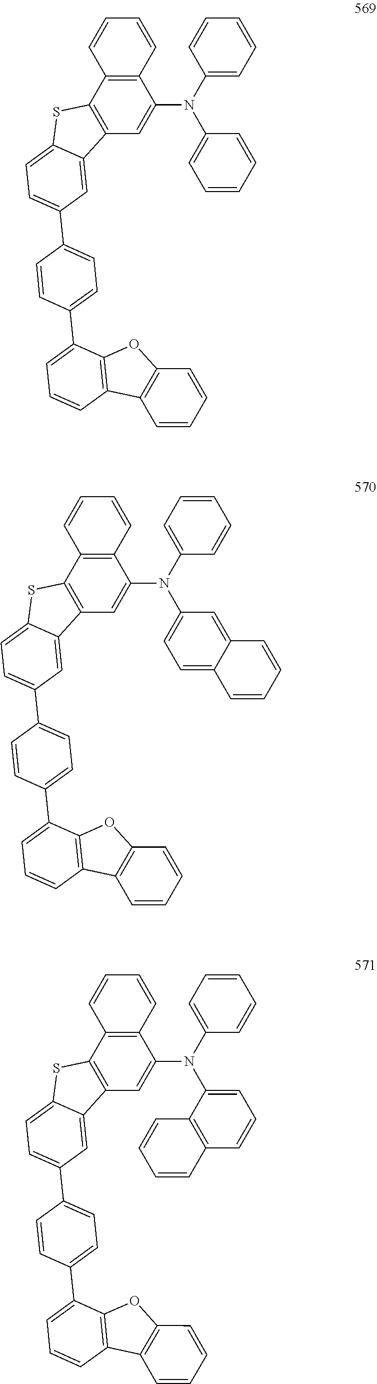 [SMILES: c1ccc(N(c2ccccc2)c2cc3c4cc(-c5ccc(-c6cccc7c6oc6ccccc67)cc5)ccc4sc3c3ccccc23)cc1 ; c1ccc(N(c2ccc3ccccc3c2)c2cc3c4cc(-c5ccc(-c6cccc7c6oc6ccccc67)cc5)ccc4sc3c3ccccc23)cc1 ; c1ccc(N(c2cccc3ccccc23)c2cc3c4cc(-c5ccc(-c6cccc7c6oc6ccccc67)cc5)ccc4sc3c3ccccc23)cc1]

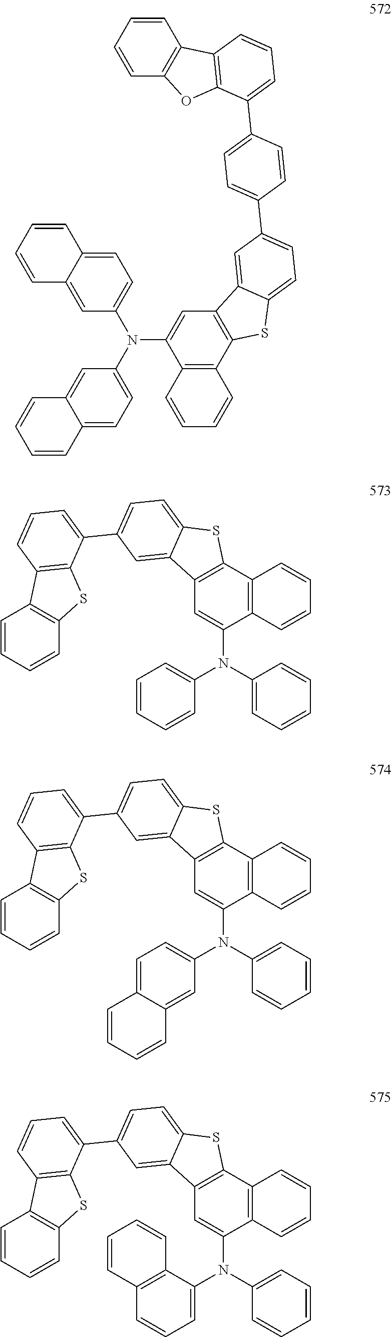 [SMILES: c1ccc2cc(N(c3ccc4ccccc4c3)c3cc4c5cc(-c6ccc(-c7cccc8c7oc7ccccc78)cc6)ccc5sc4c4ccccc34)ccc2c1 ; c1ccc(N(c2ccccc2)c2cc3c4cc(-c5cccc6c5sc5ccccc56)ccc4sc3c3ccccc23)cc1 ; c1ccc(N(c2ccc3ccccc3c2)c2cc3c4cc(-c5cccc6c5sc5ccccc56)ccc4sc3c3ccccc23)cc1 ; c1ccc(N(c2cccc3ccccc23)c2cc3c4cc(-c5cccc6c5sc5ccccc56)ccc4sc3c3ccccc23)cc1]

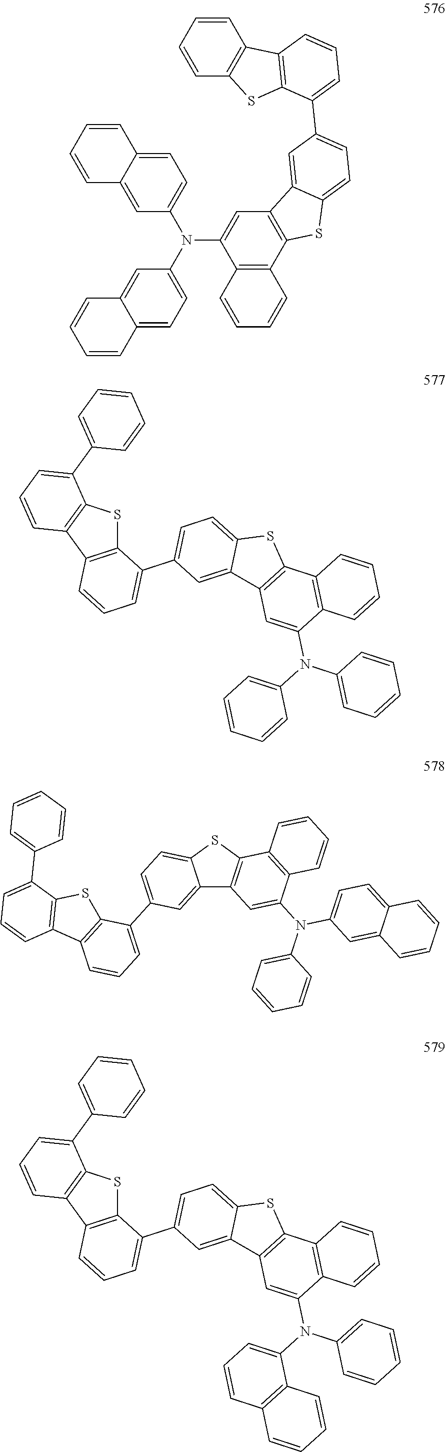

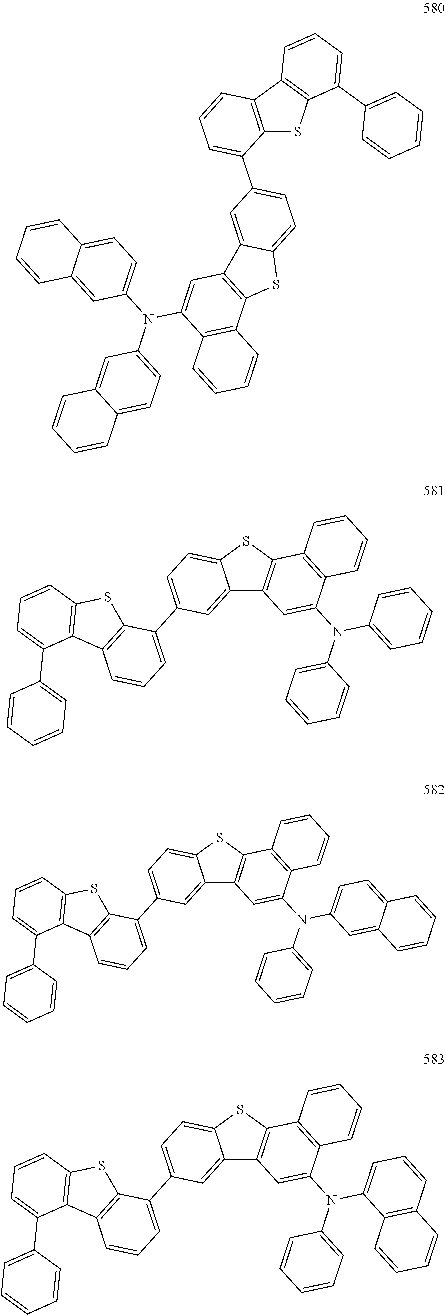 [SMILES: c1ccc(-c2cccc3c2sc2c(-c4ccc5sc6c7ccccc7c(N(c7ccc8ccccc8c7)c7ccc8ccccc8c7)cc6c5c4)cccc23)cc1 ; c1ccc(-c2cccc3sc4c(-c5ccc6sc7c8ccccc8c(N(c8ccccc8)c8ccccc8)cc7c6c5)cccc4c23)cc1 ; c1ccc(-c2cccc3sc4c(-c5ccc6sc7c8ccccc8c(N(c8ccccc8)c8ccc9ccccc9c8)cc7c6c5)cccc4c23)cc1 ; c1ccc(-c2cccc3sc4c(-c5ccc6sc7c8ccccc8c(N(c8ccccc8)c8cccc9ccccc89)cc7c6c5)cccc4c23)cc1]

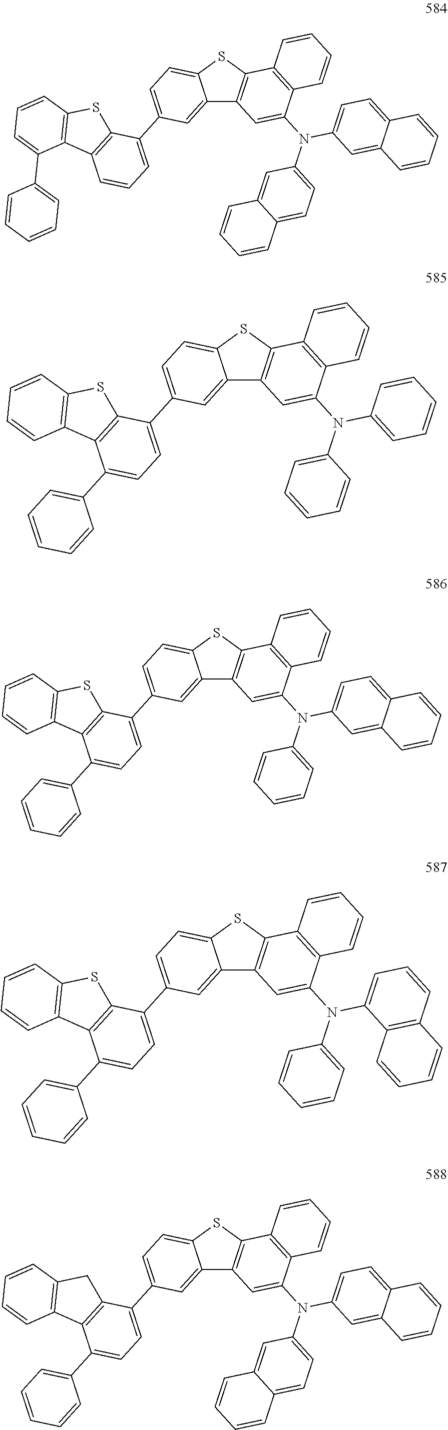 [SMILES: c1ccc(-c2cccc3sc4c(-c5ccc6sc7c8ccccc8c(N(c8ccc9ccccc9c8)c8ccc9ccccc9c8)cc7c6c5)cccc4c23)cc1 ; c1ccc(-c2ccc(-c3ccc4sc5c6ccccc6c(N(c6ccccc6)c6ccccc6)cc5c4c3)c3sc4ccccc4c23)cc1 ; c1ccc(-c2ccc(-c3ccc4sc5c6ccccc6c(N(c6ccccc6)c6ccc7ccccc7c6)cc5c4c3)c3sc4ccccc4c23)cc1 ; c1ccc(-c2ccc(-c3ccc4sc5c6ccccc6c(N(c6ccccc6)c6cccc7ccccc67)cc5c4c3)c3sc4ccccc4c23)cc1 ; c1ccc(-c2ccc(-c3ccc4sc5c6ccccc6c(N(c6ccc7ccccc7c6)c6ccc7ccccc7c6)cc5c4c3)c3c2-c2ccccc2C3)cc1]

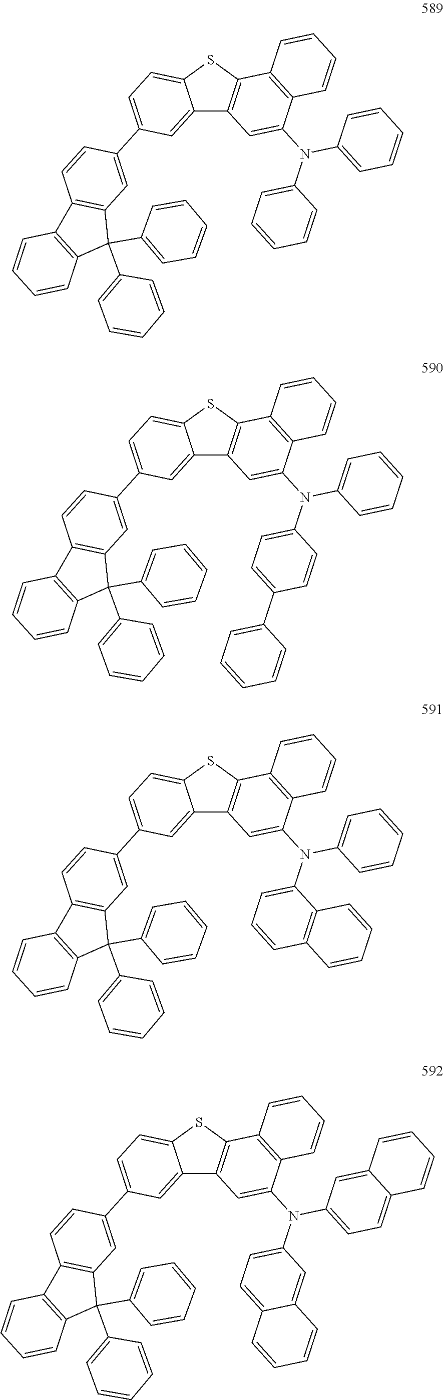 [SMILES: c1ccc(N(c2ccccc2)c2cc3c4cc(-c5ccc6c(c5)C(c5ccccc5)(c5ccccc5)c5ccccc5-6)ccc4sc3c3ccccc23)cc1 ; c1ccc(-c2ccc(N(c3ccccc3)c3cc4c5cc(-c6ccc7c(c6)C(c6ccccc6)(c6ccccc6)c6ccccc6-7)ccc5sc4c4ccccc34)cc2)cc1 ; c1ccc(N(c2cccc3ccccc23)c2cc3c4cc(-c5ccc6c(c5)C(c5ccccc5)(c5ccccc5)c5ccccc5-6)ccc4sc3c3ccccc23)cc1 ; c1ccc(C2(c3ccccc3)c3ccccc3-c3ccc(-c4ccc5sc6c7ccccc7c(N(c7ccc8ccccc8c7)c7ccc8ccccc8c7)cc6c5c4)cc32)cc1]

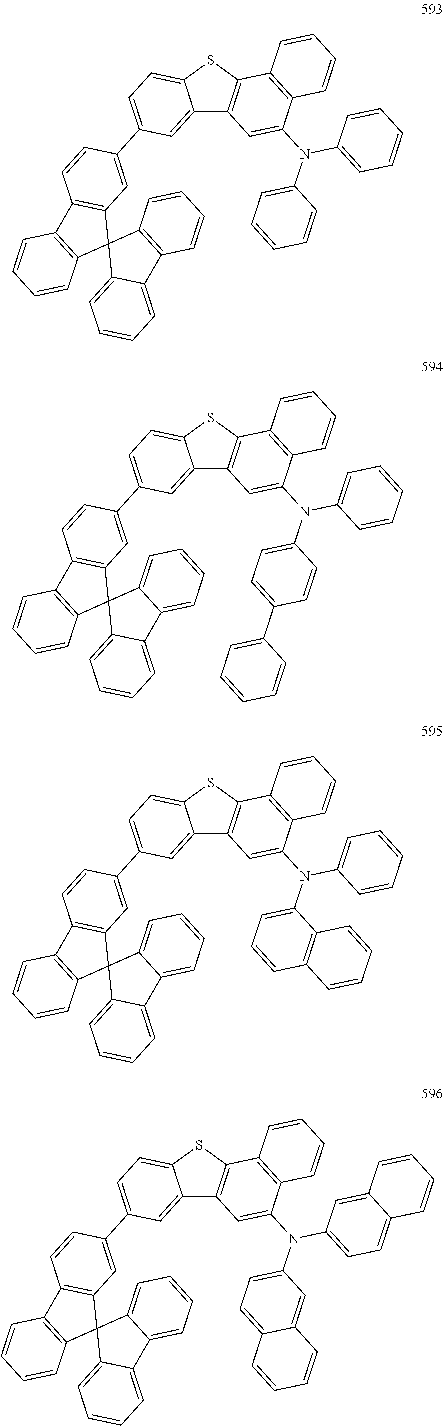 [SMILES: c1ccc(N(c2ccccc2)c2cc3c4cc(-c5ccc6c(c5)C5(c7ccccc7-c7ccccc75)c5ccccc5-6)ccc4sc3c3ccccc23)cc1 ; c1ccc(-c2ccc(N(c3ccccc3)c3cc4c5cc(-c6ccc7c(c6)C6(c8ccccc8-c8ccccc86)c6ccccc6-7)ccc5sc4c4ccccc34)cc2)cc1 ; c1ccc(N(c2cccc3ccccc23)c2cc3c4cc(-c5ccc6c(c5)C5(c7ccccc7-c7ccccc75)c5ccccc5-6)ccc4sc3c3ccccc23)cc1 ; c1ccc2c(c1)-c1ccccc1C21c2ccccc2-c2ccc(-c3ccc4sc5c6ccccc6c(N(c6ccc7ccccc7c6)c6ccc7ccccc7c6)cc5c4c3)cc21]

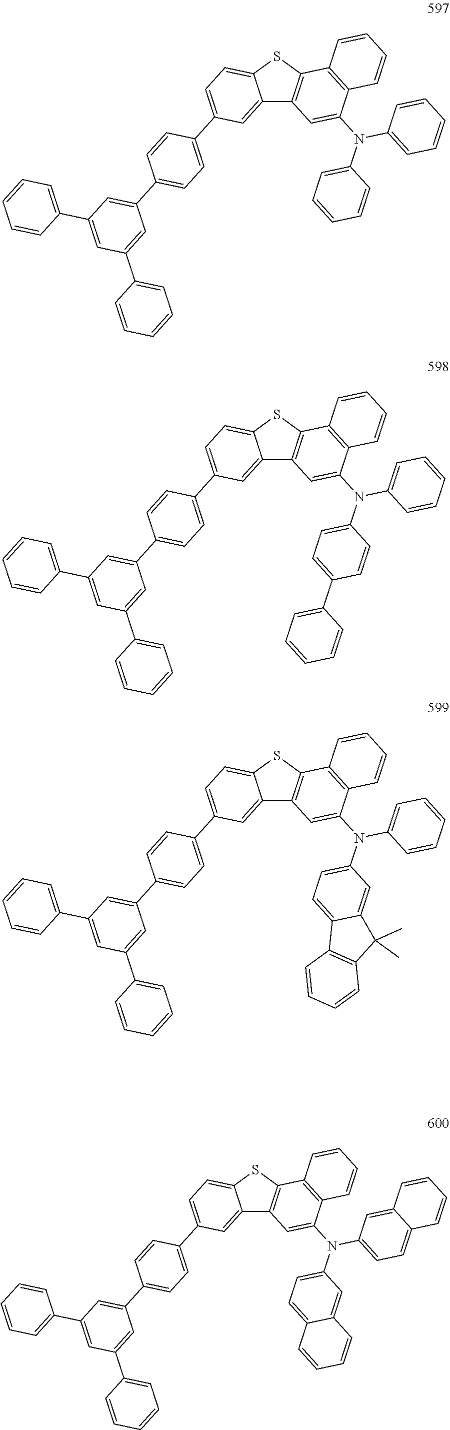 [SMILES: c1ccc(-c2cc(-c3ccccc3)cc(-c3ccc(-c4ccc5sc6c7ccccc7c(N(c7ccccc7)c7ccccc7)cc6c5c4)cc3)c2)cc1 ; c1ccc(-c2ccc(N(c3ccccc3)c3cc4c5cc(-c6ccc(-c7cc(-c8ccccc8)cc(-c8ccccc8)c7)cc6)ccc5sc4c4ccccc34)cc2)cc1 ; CC1(C)c2ccccc2-c2ccc(N(c3ccccc3)c3cc4c5cc(-c6ccc(-c7cc(-c8ccccc8)cc(-c8ccccc8)c7)cc6)ccc5sc4c4ccccc34)cc21 ; c1ccc(-c2cc(-c3ccccc3)cc(-c3ccc(-c4ccc5sc6c7ccccc7c(N(c7ccc8ccccc8c7)c7ccc8ccccc8c7)cc6c5c4)cc3)c2)cc1]

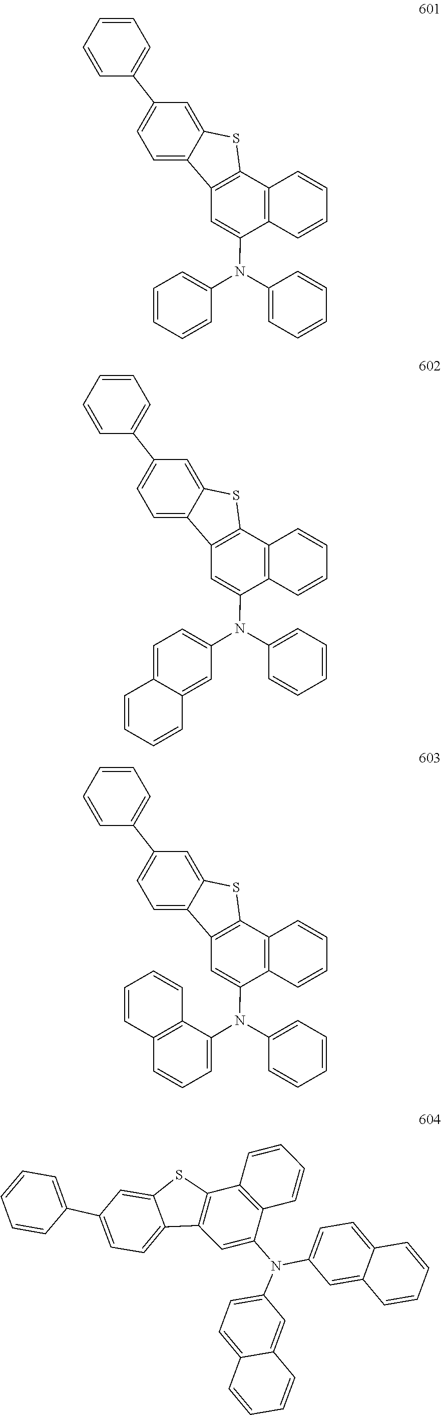

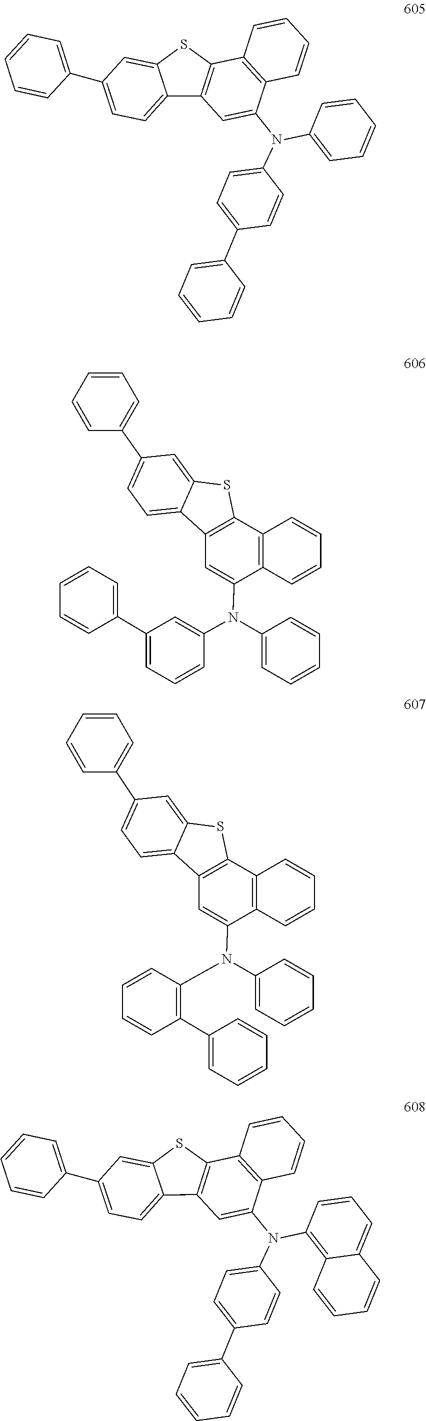

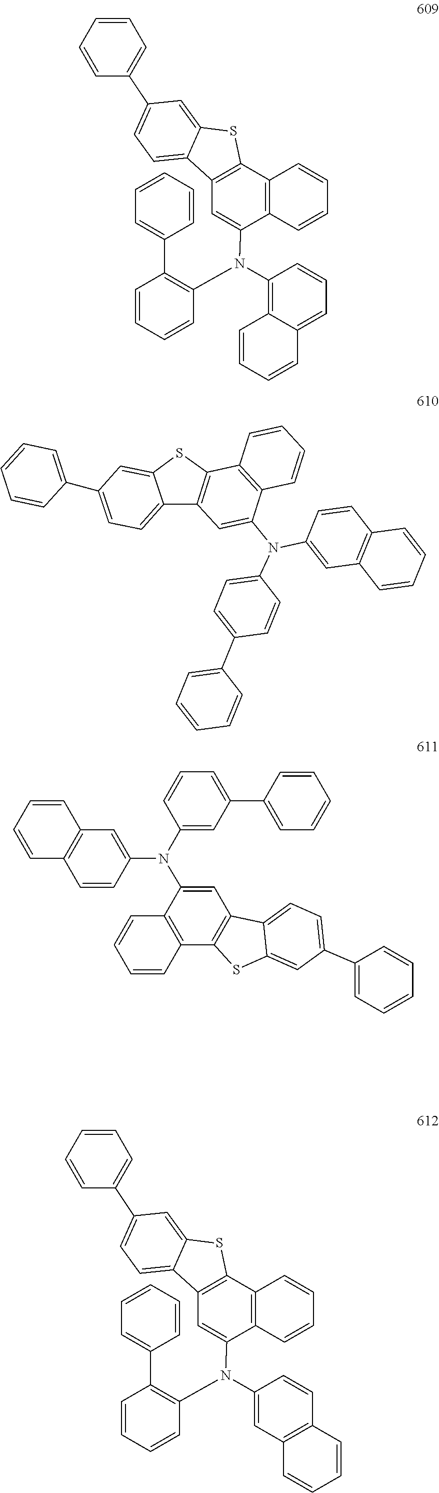

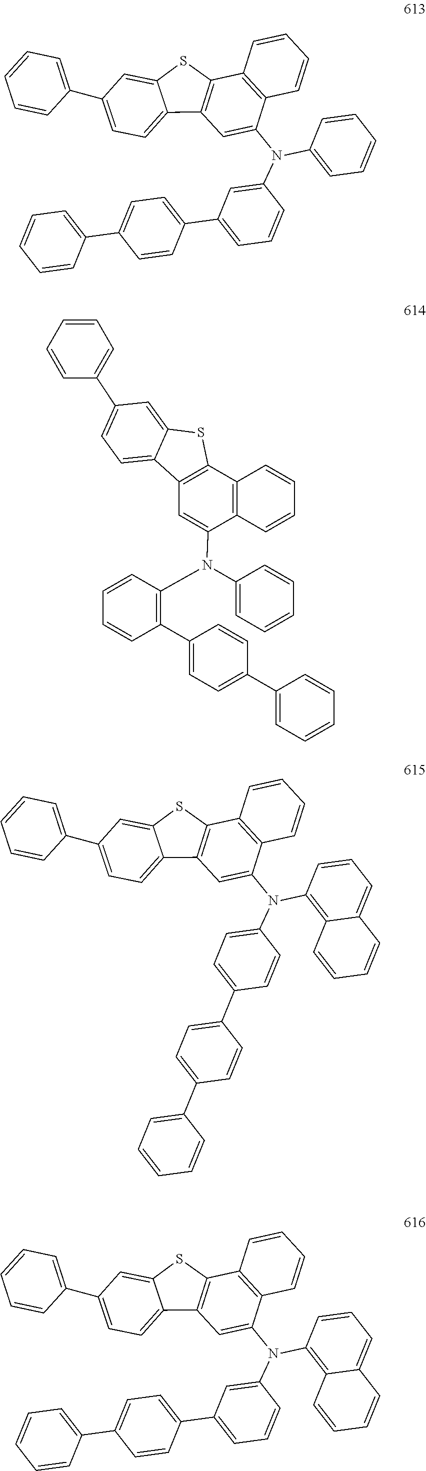

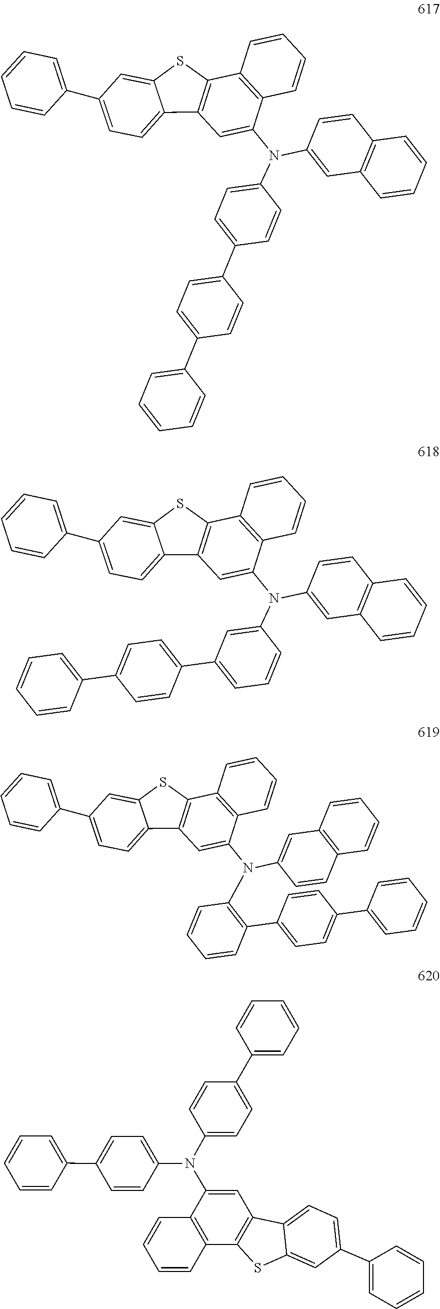 [SMILES: c1ccc(-c2ccc(-c3ccc(N(c4ccc5ccccc5c4)c4cc5c6ccc(-c7ccccc7)cc6sc5c5ccccc45)cc3)cc2)cc1 ; c1ccc(-c2ccc(-c3cccc(N(c4ccc5ccccc5c4)c4cc5c6ccc(-c7ccccc7)cc6sc5c5ccccc45)c3)cc2)cc1 ; c1ccc(-c2ccc(-c3ccccc3N(c3ccc4ccccc4c3)c3cc4c5ccc(-c6ccccc6)cc5sc4c4ccccc34)cc2)cc1 ; c1ccc(-c2ccc(N(c3ccc(-c4ccccc4)cc3)c3cc4c5ccc(-c6ccccc6)cc5sc4c4ccccc34)cc2)cc1]

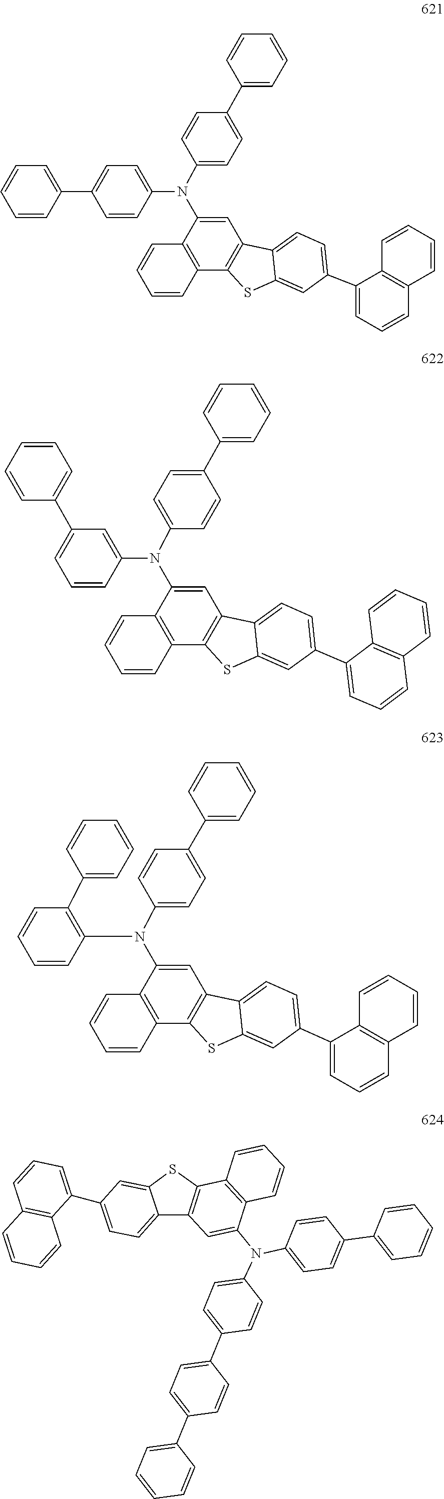 [SMILES: c1ccc(-c2ccc(N(c3ccc(-c4ccccc4)cc3)c3cc4c5ccc(-c6cccc7ccccc67)cc5sc4c4ccccc34)cc2)cc1 ; c1ccc(-c2ccc(N(c3cccc(-c4ccccc4)c3)c3cc4c5ccc(-c6cccc7ccccc67)cc5sc4c4ccccc34)cc2)cc1 ; c1ccc(-c2ccc(N(c3ccccc3-c3ccccc3)c3cc4c5ccc(-c6cccc7ccccc67)cc5sc4c4ccccc34)cc2)cc1 ; c1ccc(-c2ccc(-c3ccc(N(c4ccc(-c5ccccc5)cc4)c4cc5c6ccc(-c7cccc8ccccc78)cc6sc5c5ccccc45)cc3)cc2)cc1]

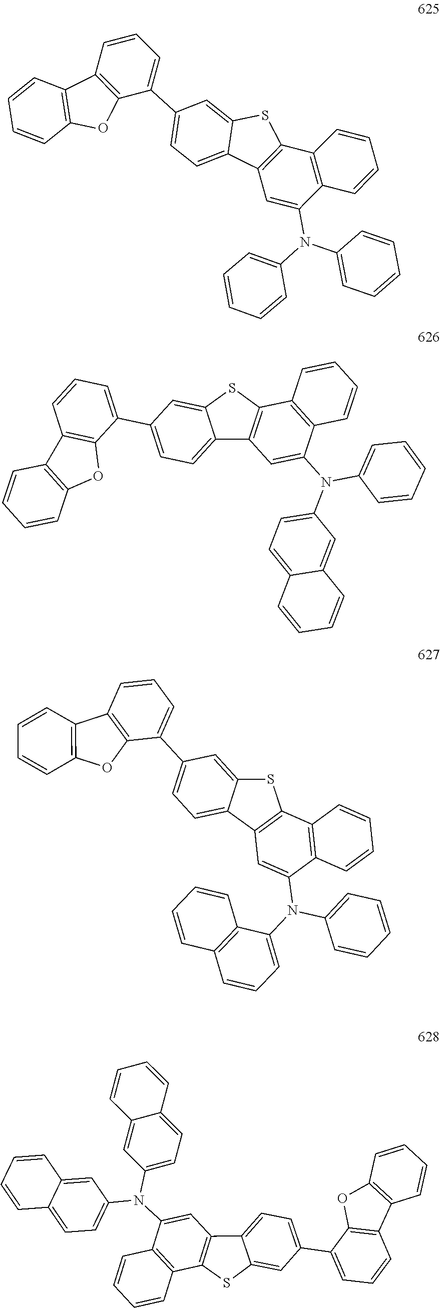 [SMILES: c1ccc(N(c2ccccc2)c2cc3c4ccc(-c5cccc6c5oc5ccccc56)cc4sc3c3ccccc23)cc1 ; c1ccc(N(c2ccc3ccccc3c2)c2cc3c4ccc(-c5cccc6c5oc5ccccc56)cc4sc3c3ccccc23)cc1 ; c1ccc(N(c2cccc3ccccc23)c2cc3c4ccc(-c5cccc6c5oc5ccccc56)cc4sc3c3ccccc23)cc1 ; c1ccc2cc(N(c3ccc4ccccc4c3)c3cc4c5ccc(-c6cccc7c6oc6ccccc67)cc5sc4c4ccccc34)ccc2c1]

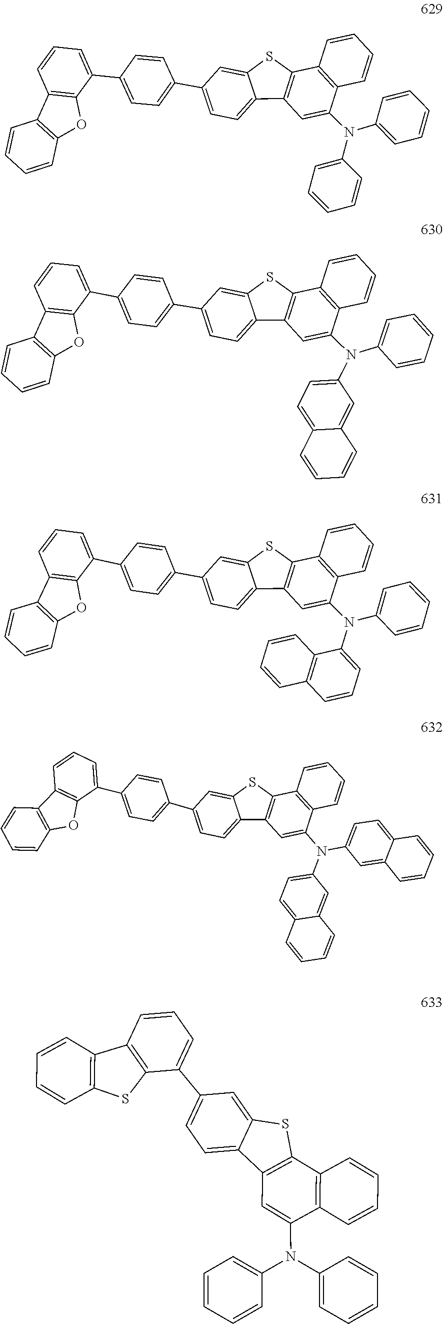 [SMILES: c1ccc(N(c2ccccc2)c2cc3c4ccc(-c5ccc(-c6cccc7c6oc6ccccc67)cc5)cc4sc3c3ccccc23)cc1 ; c1ccc(N(c2ccc3ccccc3c2)c2cc3c4ccc(-c5ccc(-c6cccc7c6oc6ccccc67)cc5)cc4sc3c3ccccc23)cc1 ; c1ccc(N(c2cccc3ccccc23)c2cc3c4ccc(-c5ccc(-c6cccc7c6oc6ccccc67)cc5)cc4sc3c3ccccc23)cc1 ; c1ccc2cc(N(c3ccc4ccccc4c3)c3cc4c5ccc(-c6ccc(-c7cccc8c7oc7ccccc78)cc6)cc5sc4c4ccccc34)ccc2c1 ; c1ccc(N(c2ccccc2)c2cc3c4ccc(-c5cccc6c5sc5ccccc56)cc4sc3c3ccccc23)cc1]

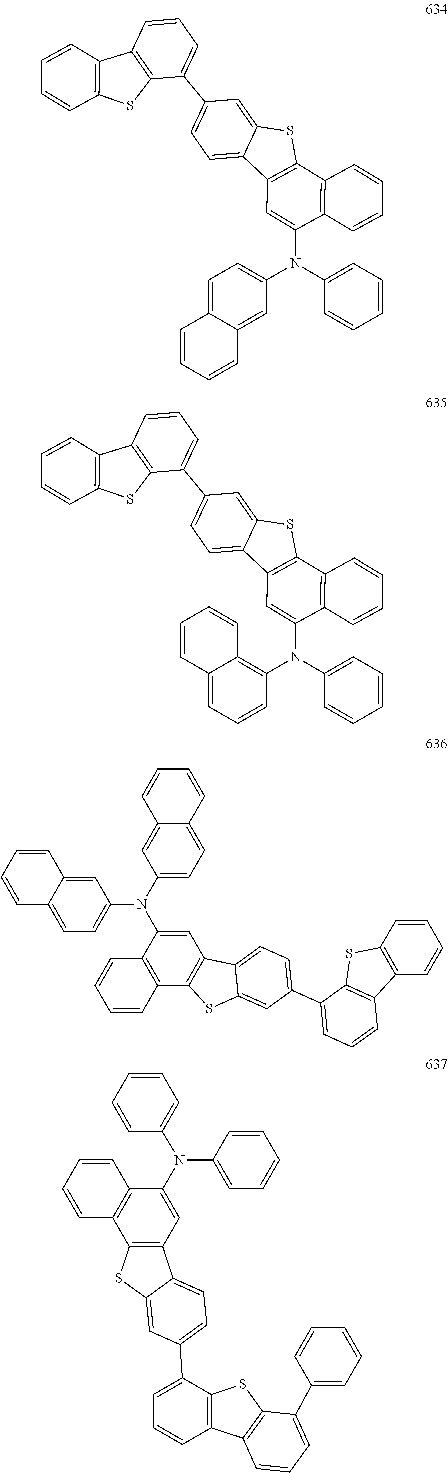

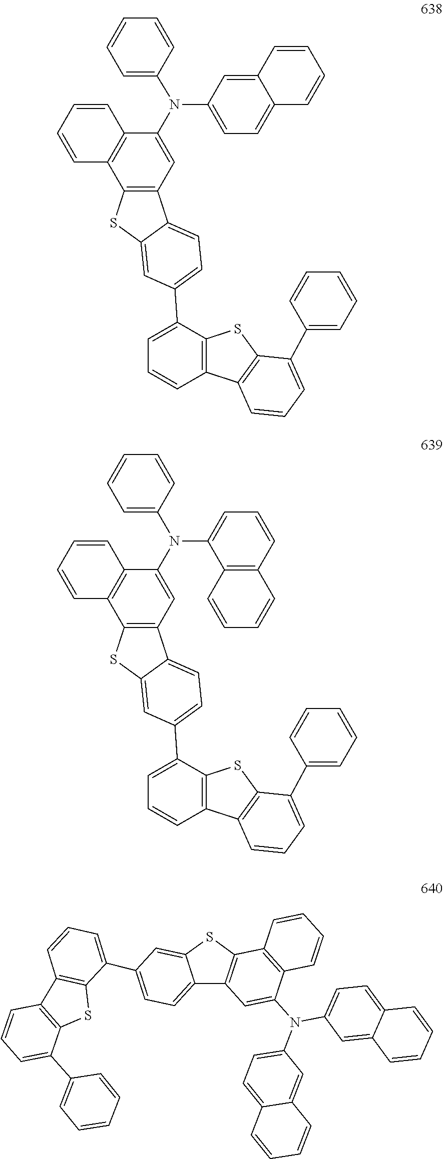 [SMILES: c1ccc(-c2cccc3c2sc2c(-c4ccc5c(c4)sc4c6ccccc6c(N(c6ccccc6)c6ccc7ccccc7c6)cc54)cccc23)cc1 ; c1ccc(-c2cccc3c2sc2c(-c4ccc5c(c4)sc4c6ccccc6c(N(c6ccccc6)c6cccc7ccccc67)cc54)cccc23)cc1 ; c1ccc(-c2cccc3c2sc2c(-c4ccc5c(c4)sc4c6ccccc6c(N(c6ccc7ccccc7c6)c6ccc7ccccc7c6)cc54)cccc23)cc1]

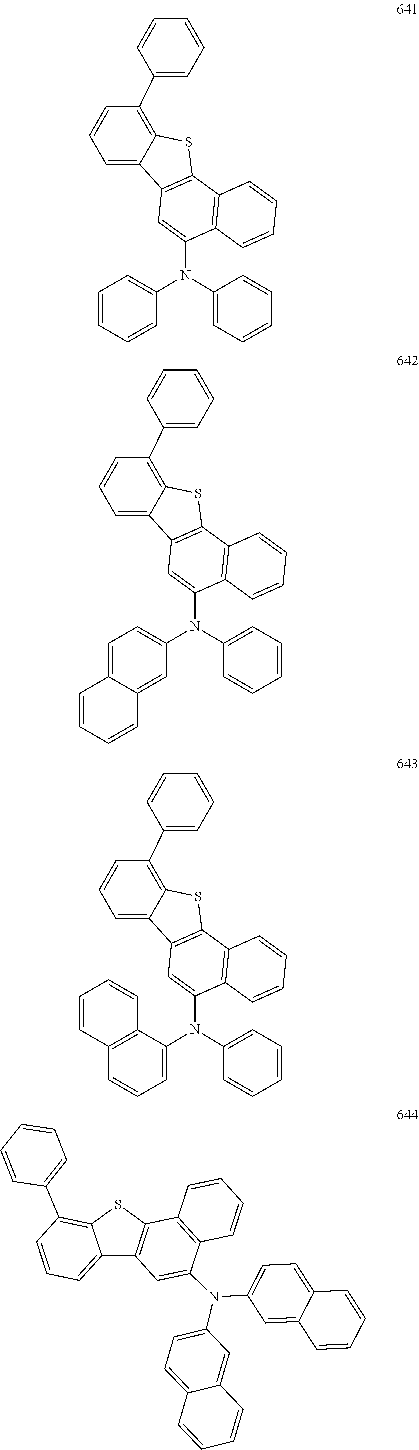

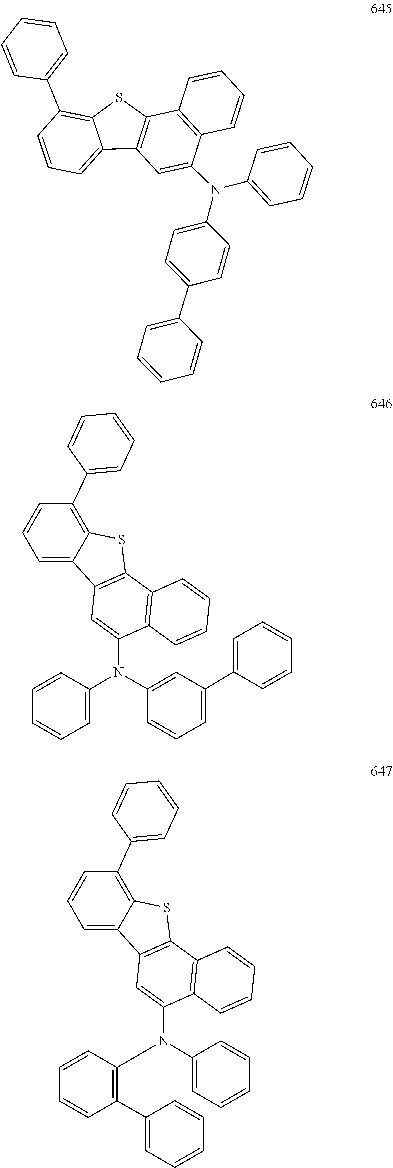

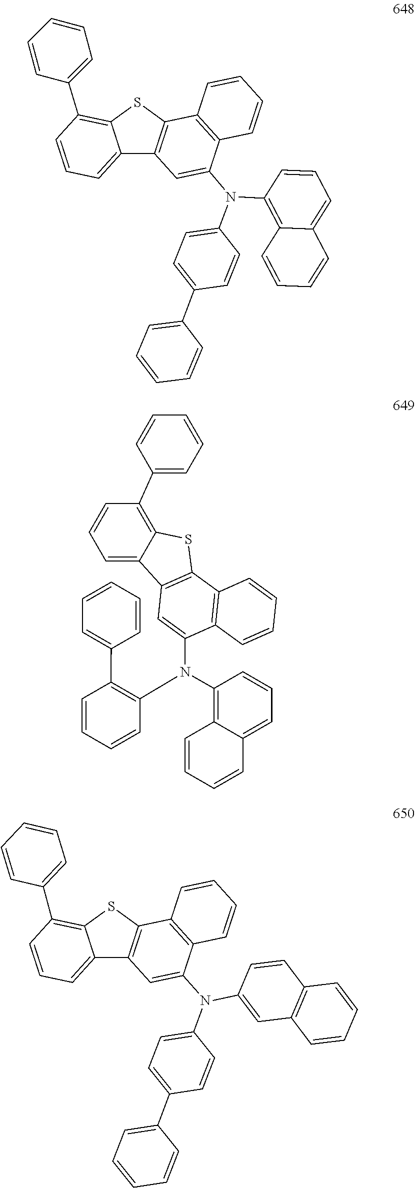

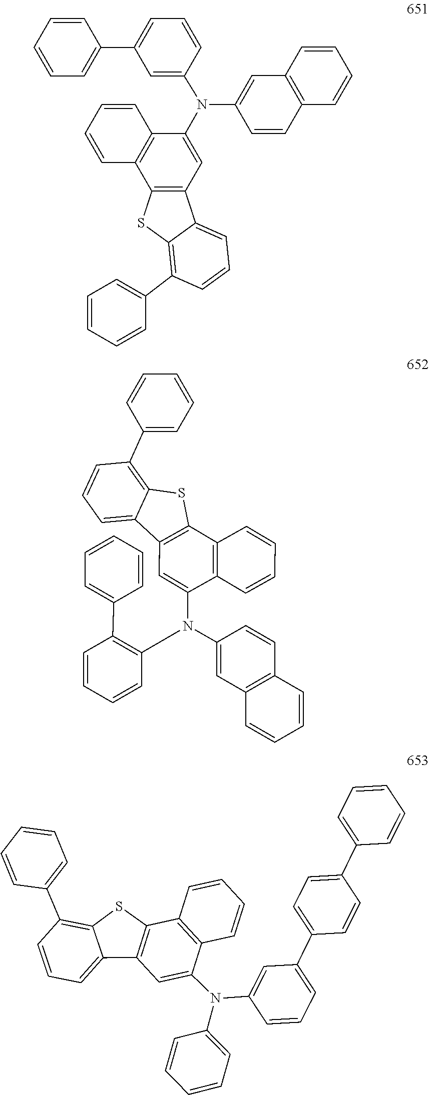

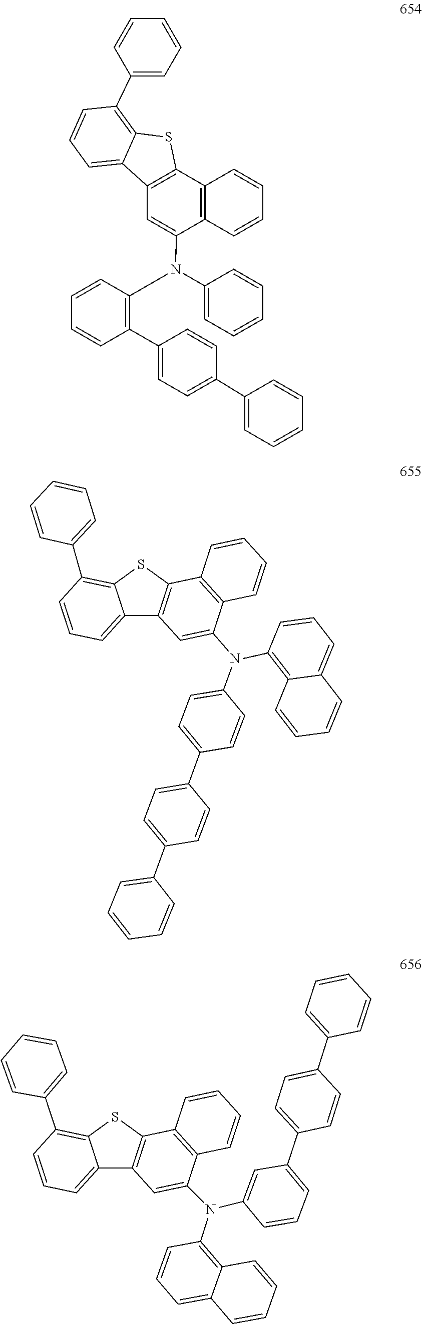 [SMILES: c1ccc(-c2ccc(-c3ccccc3N(c3ccccc3)c3cc4c5cccc(-c6ccccc6)c5sc4c4ccccc34)cc2)cc1 ; c1ccc(-c2ccc(-c3ccc(N(c4cccc5ccccc45)c4cc5c6cccc(-c7ccccc7)c6sc5c5ccccc45)cc3)cc2)cc1 ; c1ccc(-c2ccc(-c3cccc(N(c4cccc5ccccc45)c4cc5c6cccc(-c7ccccc7)c6sc5c5ccccc45)c3)cc2)cc1]

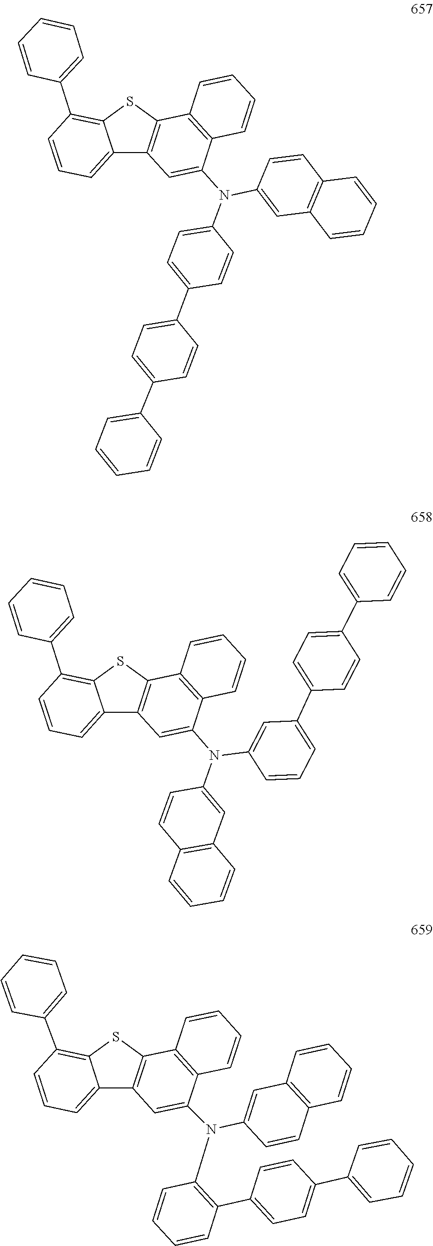 [SMILES: c1ccc(-c2ccc(-c3ccc(N(c4ccc5ccccc5c4)c4cc5c6cccc(-c7ccccc7)c6sc5c5ccccc45)cc3)cc2)cc1 ; c1ccc(-c2ccc(-c3cccc(N(c4ccc5ccccc5c4)c4cc5c6cccc(-c7ccccc7)c6sc5c5ccccc45)c3)cc2)cc1 ; c1ccc(-c2ccc(-c3ccccc3N(c3ccc4ccccc4c3)c3cc4c5cccc(-c6ccccc6)c5sc4c4ccccc34)cc2)cc1]

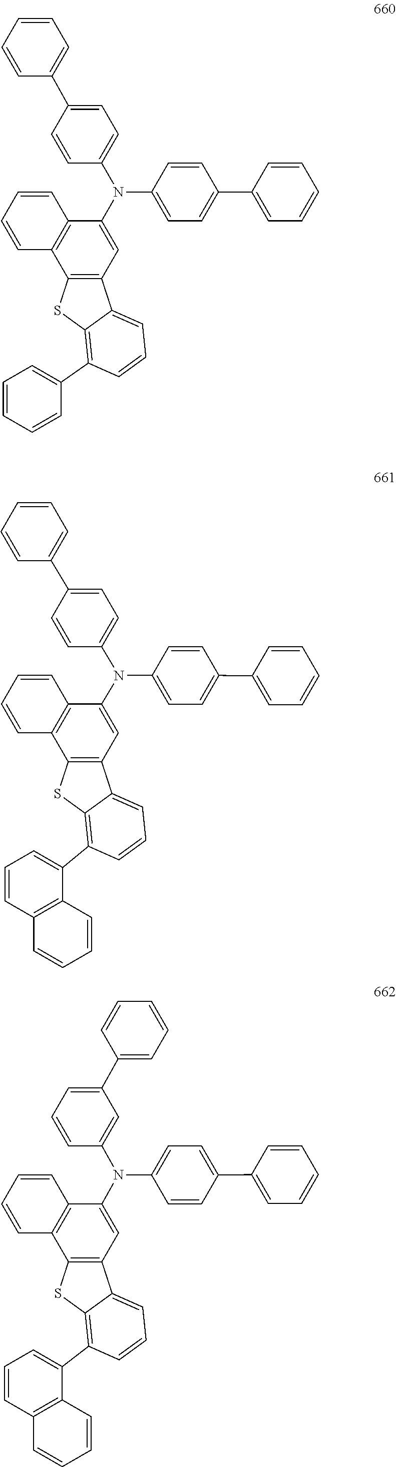 [SMILES: c1ccc(-c2ccc(N(c3ccc(-c4ccccc4)cc3)c3cc4c5cccc(-c6ccccc6)c5sc4c4ccccc34)cc2)cc1 ; c1ccc(-c2ccc(N(c3ccc(-c4ccccc4)cc3)c3cc4c5cccc(-c6cccc7ccccc67)c5sc4c4ccccc34)cc2)cc1 ; c1ccc(-c2ccc(N(c3cccc(-c4ccccc4)c3)c3cc4c5cccc(-c6cccc7ccccc67)c5sc4c4ccccc34)cc2)cc1]

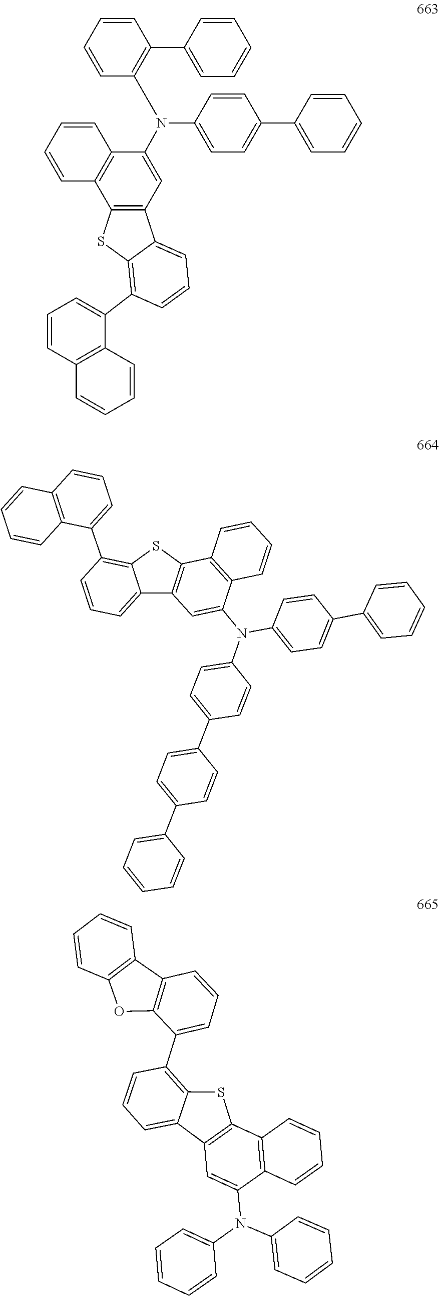

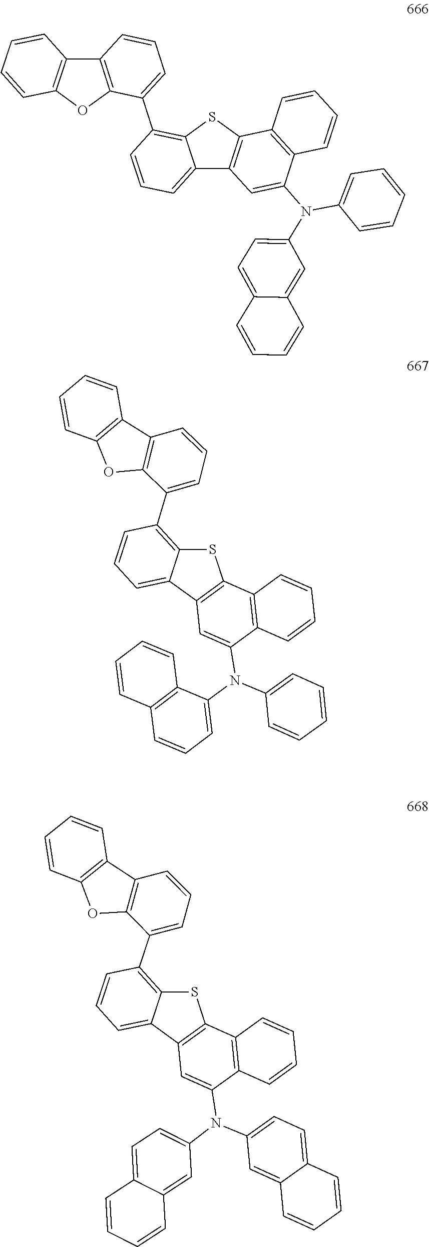 [SMILES: c1ccc(N(c2ccc3ccccc3c2)c2cc3c4cccc(-c5cccc6c5oc5ccccc56)c4sc3c3ccccc23)cc1 ; c1ccc(N(c2cccc3ccccc23)c2cc3c4cccc(-c5cccc6c5oc5ccccc56)c4sc3c3ccccc23)cc1 ; c1ccc2cc(N(c3ccc4ccccc4c3)c3cc4c5cccc(-c6cccc7c6oc6ccccc67)c5sc4c4ccccc34)ccc2c1]

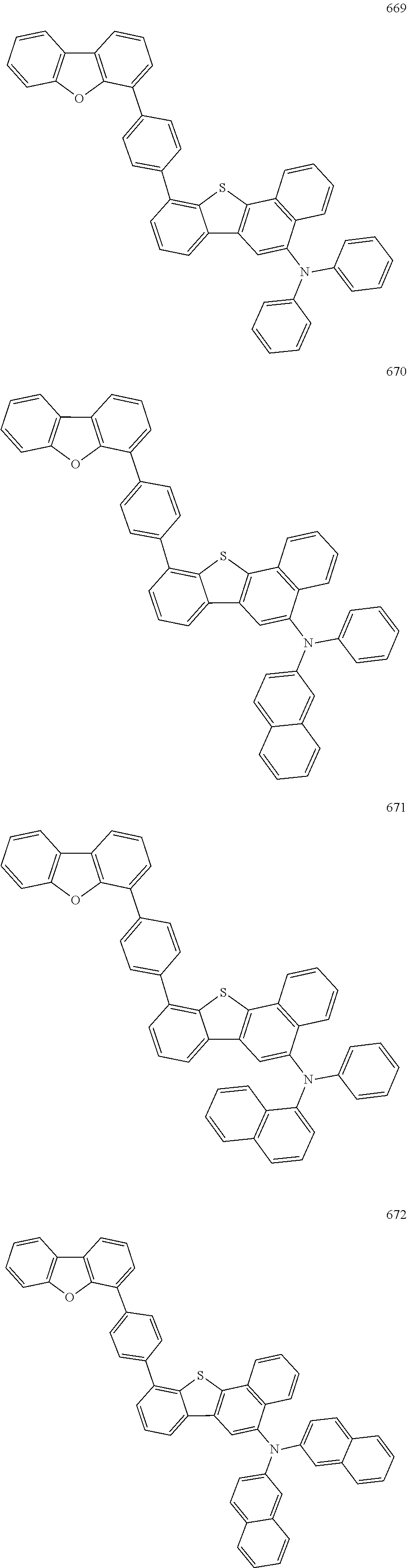 [SMILES: c1ccc(N(c2ccccc2)c2cc3c4cccc(-c5ccc(-c6cccc7c6oc6ccccc67)cc5)c4sc3c3ccccc23)cc1 ; c1ccc(N(c2ccc3ccccc3c2)c2cc3c4cccc(-c5ccc(-c6cccc7c6oc6ccccc67)cc5)c4sc3c3ccccc23)cc1 ; c1ccc(N(c2cccc3ccccc23)c2cc3c4cccc(-c5ccc(-c6cccc7c6oc6ccccc67)cc5)c4sc3c3ccccc23)cc1 ; c1ccc2cc(N(c3ccc4ccccc4c3)c3cc4c5cccc(-c6ccc(-c7cccc8c7oc7ccccc78)cc6)c5sc4c4ccccc34)ccc2c1]

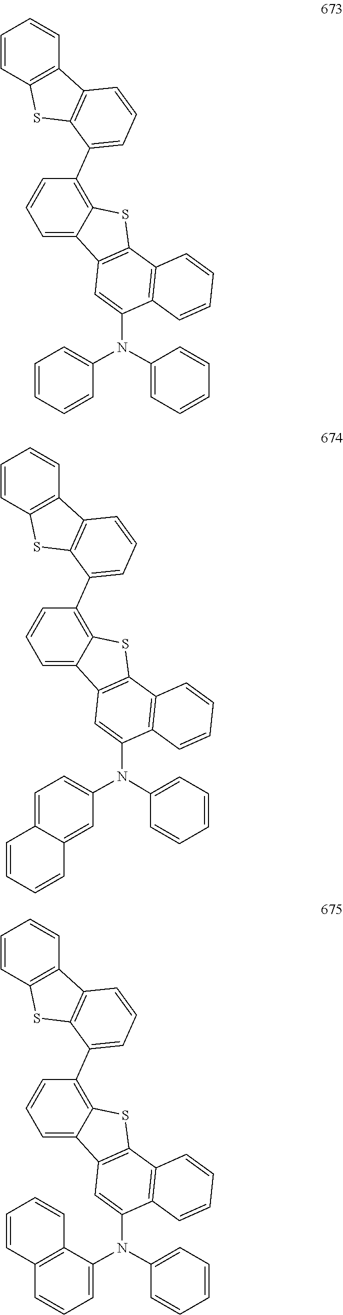

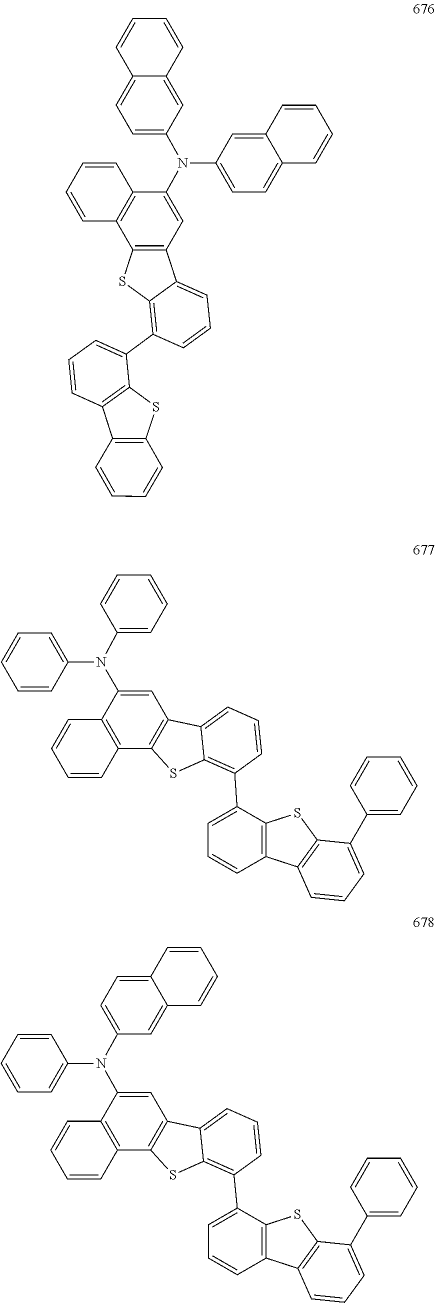 [SMILES: c1ccc2cc(N(c3ccc4ccccc4c3)c3cc4c5cccc(-c6cccc7c6sc6ccccc67)c5sc4c4ccccc34)ccc2c1 ; c1ccc(-c2cccc3c2sc2c(-c4cccc5c4sc4c6ccccc6c(N(c6ccccc6)c6ccccc6)cc54)cccc23)cc1 ; c1ccc(-c2cccc3c2sc2c(-c4cccc5c4sc4c6ccccc6c(N(c6ccccc6)c6ccc7ccccc7c6)cc54)cccc23)cc1]

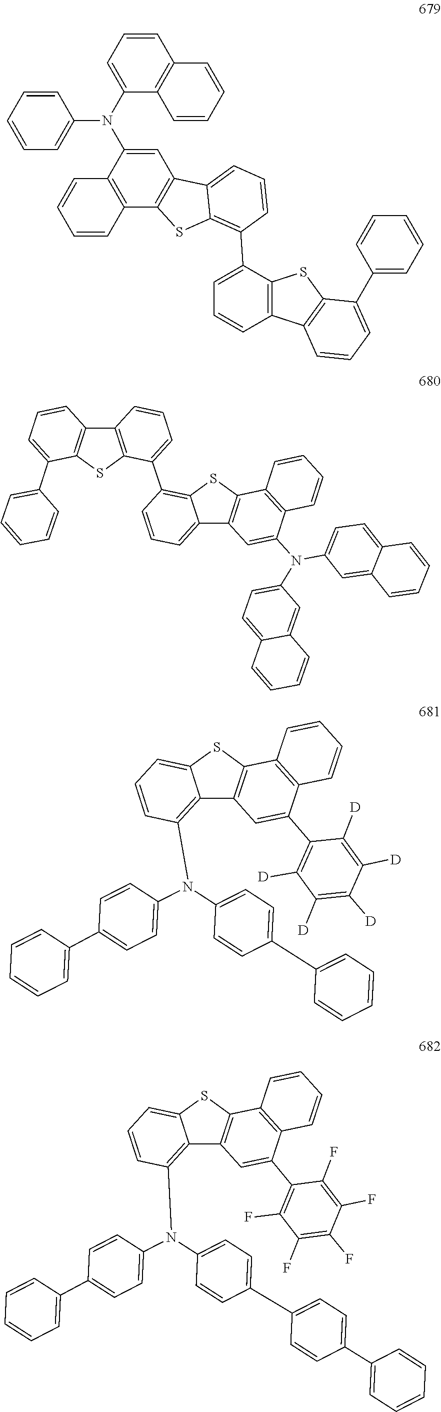

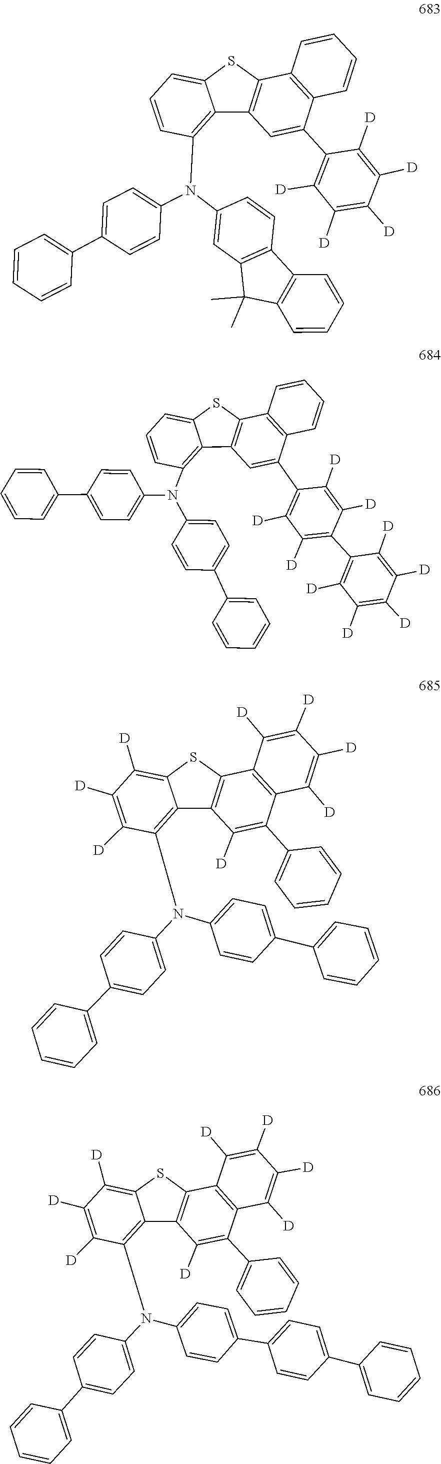

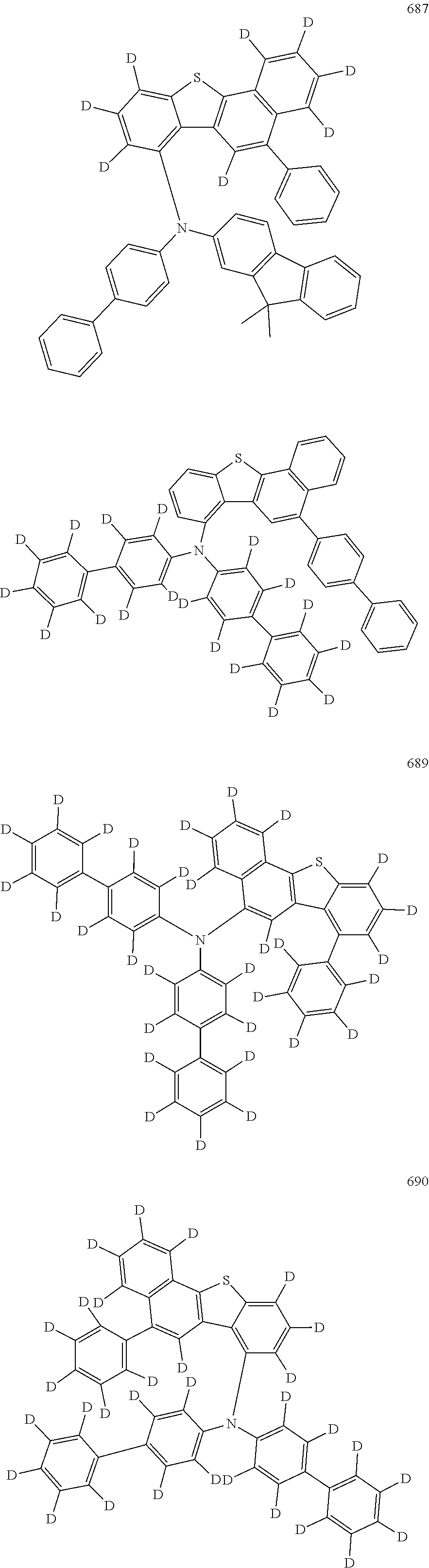

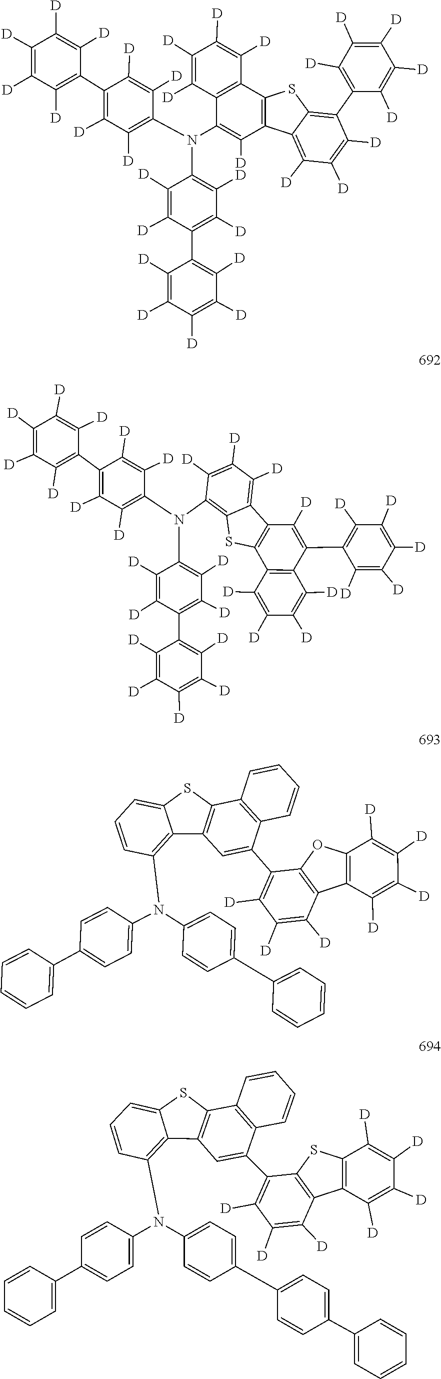

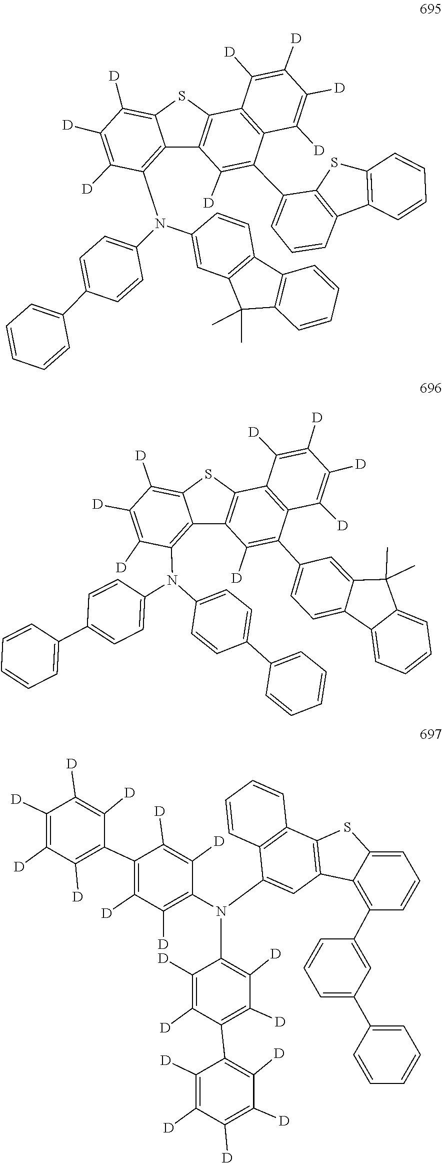 [SMILES: [2H]c1c([2H])c(N(c2ccc(-c3ccccc3)cc2)c2ccc3c(c2)C(C)(C)c2ccccc2-3)c2c(sc3c4c([2H])c([2H])c([2H])c([2H])c4c(-c4cccc5c4sc4ccccc45)c([2H])c32)c1[2H] ; [2H]c1c([2H])c(N(c2ccc(-c3ccccc3)cc2)c2ccc(-c3ccccc3)cc2)c2c(sc3c4c([2H])c([2H])c([2H])c([2H])c4c(-c4ccc5c(c4)C(C)(C)c4ccccc4-5)c([2H])c32)c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4c(sc5cccc(-c6cccc(-c7ccccc7)c6)c54)c4ccccc34)c([2H])c2[2H])c([2H])c1[2H]]

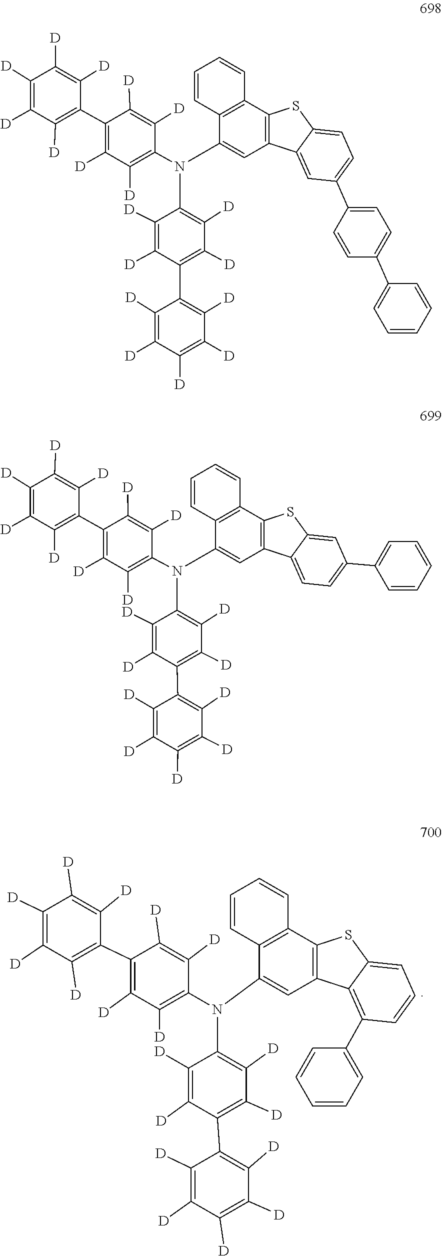

8. An organic light emitting device comprising:
a first electrode;
a second electrode provided opposite to the first electrode; and
one or more organic material layers provided between the first electrode and the second electrode,
wherein at least one of the one or more layers of the organic material layers comprise the heterocyclic compound of claim 1.

9. The organic light emitting device of claim 8, wherein the organic material layer comprises a hole transport layer, and the hole transport layer comprises the heterocyclic compound.

10. The organic light emitting device of claim 8, wherein the organic material layer comprises an electron blocking layer, and the electron blocking layer comprises the heterocyclic compound.

11. The organic light emitting device of claim 8, wherein the organic material layer comprises a light emitting auxiliary layer, and the light emitting auxiliary layer comprises the heterocyclic compound.

12. The organic light emitting device of claim 8, wherein the organic material layer comprises an electron injection layer, a hole injection layer, an electron transport layer or a hole blocking layer, and the electron injection layer, the hole injection layer, the electron transport layer or the hole blocking layer comprises the heterocyclic compound.

13. The organic light emitting device of claim 8, comprising one or more selected from the group consisting of a light emitting layer, a hole injection layer, a hole transport layer, a hole blocking layer, an electron injection layer, an electron transport layer and an electron blocking layer.
